(19) United States
(12) Patent Application Publication
(10) Pub. No.: US 2020/0069814 A1
(43) Pub. Date: Mar. 5, 2020

(54) CONJUGATION OF A CYTOTOXIC DRUG WITH BIS-LINKAGE

(71) Applicant: Hangzhou DAC Biotech Co., Ltd., Hangzhou (CN)

(72) Inventors: Robert Yongxin ZHAO, Lexington, MA (US); Yuanyuan HUANG, Hangzhou (CN); Qingliang YANG, Hangzhou (CN); Shun GAI, Hangzhou (CN); Hangbo YE, Hangzhou (CN); Linyao ZHAO, Hangzhou (CN); Chengyu YANG, Hangzhou (CN); Yifang XU, Hangzhou (CN); Huihui GUO, Hangzhou (CN); Minjun CHAO, Hangzhou (CN); Qianqian TONG, Hangzhou (CN); Wenjun LI, Hangzhou (CN); Xiang CAI, Hangzhou (CN); Xiaomai ZHOU, Hangzhou (CN); Hongsheng XIE, Hangzhou (CN); Junxiang JIA, Hangzhou (CN); Haifeng ZHU, Hangzhou (CN); Zhixiang GUO, Hangzhou (CN); Shuihong GAO, Hangzhou (CN); Chunyan WANG, Hangzhou (CN); Chen LIN, Hangzhou (CN); Yanlei YANG, Hangzhou (CN); Zhicang YE, Hangzhou (CN); Jie PENG, Hangzhou (CN); Jun XU, Hangzhou (CN); Xiaotao ZUO, Hangzhou (CN); Qingyu SU, Hangzhou (CN)

(73) Assignee: Hangzhou DAC Biotech Co., Ltd., Hangzhou (CN)

(21) Appl. No.: 16/488,764

(22) PCT Filed: Apr. 6, 2017
(86) PCT No.: PCT/IB2017/051977

§ 371 Date: Aug. 26, 2019

(51) Int. Cl. A61K 47/68

(52) U.S. Cl. CPC A61K 47/6803; A61K 45/06; A61K 47/6889

(57) ABSTRACT

A conjugation of a cytotoxic drug to a cell-binding molecule with a bis-linker (dual-linker) as shown in Formula (I). Bis-linkage methods of making a conjugate of a cytotoxic drug/molecule to a cell-binding agent in a specific manner are also described, as well as application of the conjugates for the treatment of a cancer, or an autoimmune disease, or an infectious disease.

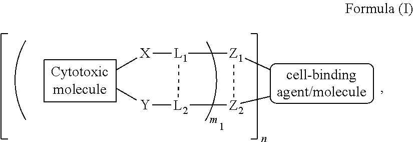

wherein “ [symbol] ” is an optional bond; X, Y, Z₁, and Z₂ are a functional group; m₁ and n are a integer; L₁ and L₂ are a linker.

### FIELD OF THE INVENTION

The present invention relates to the conjugation of cytotoxic to a cell-binding molecule with a bis-linker (dual-linker). It relates to a bis-linkage method of conjugation of a cytotoxic drug/molecule, particularly when the drug having dual functional groups of amino, hydroxyl, diamino, amino-hydroxyl, dihydroxyl, carboxyl, hydrazine, aldehyde and thiol. The present invention also relates to methods of making cell-binding agent-drug (cytotoxic agent) conjugates with the bis-linker in a specific manner.

### BACKGROUND OF THE INVENTION

Antibody-drug conjugates (ADCs) have become one of promising targeting therapies for cancer as evidenced by the clinical success of brentuximab vedotin (Adcetris) for relapsed/refractory Hodgkin lymphoma (Okeley, N., et al, Hematol Oncol. Clin. North. Am, 2014, 28, 13-25; Gopal, A., et al, Blood 2015, 125, 1236-43) and ado-trastuzumab emtansine for relapsed HER2+ breast cancer (Peddi, P. and Hurvitz, S., Ther. Adv. Med. Oncol. 2014, 6(5), 202-9; Lambert, J. and Chari, R., J. Med. Chem. 2014, 57, 6949-64). The three important components, monoclonal antibody, cytotoxic payload, and conditional linker of ADCs plus the sites where to link the linker-payload components are all important factors to make success of ADC. It has be three decades to study each factor of the components of ADCs. However, linker technologies remain limited in scope, since drugs that are conjugated must contain certain reactive functional groups, ensure circulation stability, and facile drug release upon antigen binding and intracellular uptake, and importantly be not harming normal tissues once the linker-payload components are off-targeted during the circulation (Ponte, J. et al., Bioconj. Chem., 2016, 27(7), 1588-98; Dovgan, I., et al. Sci. Rep. 2016, 6, 30835; Ross, P. L. and Wolfe, J. L. J. Pharm. Sci. 105(2), 391-7; Chen, T. et al. J. Pharm. Biomed. Anal., 2016, 117, 304-10).

In early ADCs, the linkers which were particularly used for ADCs targeting of liquid tumor were too labile, and led to the release of free drug in the circulation and consequent off-target toxicity (Bander, N. H. et al, Clin. Adv. Hematol. Oncol., 2012, 10, 1-16). In the current generation of ADCs, the linkers are more stable, and the cytotoxic agents are significantly more potent (Behrens, C. R. and Liu, B., mAbs, 2014. 6, 46-53). However, the off-target toxicity so far is still the major challenge in development of ADC drugs (Roberts, S. A. et al, Regul. Toxicol. Pharmacol. 2013, 67, 382-91). For instance, in clinical practice Ado-trastuzumab emtansine (T-DM1, Kadcyla®) which is used stable (none-cleavable) MCC linker has shown great benefit to patients who have HER2-positive metastatic breast cancer (mBC) or who have already been treated for mBC or developed HER2 tumor recurrence within six months of adjuvant therapy (Peddi, P. and Hurvitz, S., Ther. Adv. Med. Oncol. 2014, 6(5), 202-209; Piwko C, et al, Clin Drug Investig. 2015, 35(8), 487-93; Lambert, J. and Chari, R., J. Med. Chem. 2014, 57, 6949-64). But, T-DM1 had failed in clinic trial as first-line treatment for patients with HER2 positive unresectable locally advanced or metastatic breast cancer and as the second line treatment of HER2-positive advanced gastric cancer due to a little benefit to patients when comparison the side toxicity to the efficacy (Ellis, P. A., et al, J. Clin. Oncol. 2015, 33, (suppl; abstr 507 of 2015 ASCO Annual Meeting); Shen, K. et al, Sci Rep. 2016; 6: 23262; de Goeij, B. E. and Lambert, J. M. Curr Opin Immunol 2016, 40, 14-23; Barrios, C. H. et al, J Clin Oncol 2016, 34, (suppl; abstr 593 of 2016 ASCO Annual Meeting).

To address issues of the off-target toxicity, research and development into ADC chemistry and design are now expanding the scopes of the linker-payload compartments and conjugate chemistry beyond the sole potent payloads, and especially to address activity of the linker-payload of ADCs toward targets/target diseases (Lambert, J. M. Ther Deliv 2016, 7, 279-82; Zhao, R. Y. et al, 2011, J. Med. Chem. 54, 3606-23). Nowadays many drug developers and academic institutions are highly focusing on establishing novel reliable specific conjugation linkers and methods for site-specific ADC conjugation, which seem to have longer circulation half-life, higher efficacy, potentially decreased off-target toxicity, and a narrow range of in vivo pharmacokinetic (PK) properties of ADCs as well as better batch-to-batch consistency in ADC production (Hamblett, K. J. et al, Clin. Cancer Res. 2004, 10, 7063-70; Adem, Y. T. et al, Bioconjugate Chem. 2014, 25, 656-664; Boylan, N. J. Bioconjugate Chem. 2013, 24, 1008-1016; Strop, P., et al 2013 Chem. Biol. 20, 161-67; Wakankar, A. mAbs, 2011, 3, 161-172). These specific conjugation methods reported so far include incorporation of engineered cysteines (Junutula, J. R. et al. Nat. Biotechnol. 2008, 26, 925-32; Junutula, J. R., et al 2010 Clin. Cancer Res. 16, 4769; U.S. Pat. Nos. 8,309,300; 7,855,275; 7,521,541; 7,723,485, WO2008/141044), selenocysteines (Hofer, T., et al. Biochemistry 2009, 48, 12047-57; Li, X., et al. Methods 2014, 65, 133-8; U.S. Pat. No. 8,916,159 for US National Cancer Institute), cysteine containing tag with perfluoroaromatic reagents (Zhang, C. et al. Nat. Chem. 2015, 8, 1-9), thiolfucose (Okeley, N. M., et al 2013 Bioconjugate Chem. 24, 1650), non-natural amino acids (Axup, J. Y., et al, Proc. Nat. Acad. Sci. USA. 2012, 109, 16101-6; Zimmerman, E. S., et al., 2014, Bioconjug. Chem. 25, 351-361; Wu, P., et al, 2009 Proc. Natl. Acad. Sci. 106, 3000-5; Rabuka, D., et al, Nat. Protoc. 2012, 7, 1052-67; U.S. Pat. No. 8,778,631 and US Pat Appl. 20100184135, WO2010/081110 for Sutro Biopharma; WO2006/069246, 2007/059312, U.S. Pat. Nos. 7,332,571, 7,696,312, and 7,638,299 for Ambrx; WO2007/130453, U.S. Pat. Nos. 7,632,492 and 7,829,659 for Allozyne), conjugation to reduced intermolecular disulfides by re-bridging dibromomalemides (Jones, M. W. et al. J. Am. Chem. Soc. 2012, 134, 1847-52), bis-sulfone reagents (Badescu, G. et al. Bioconjug. Chem. 2014, 25, 1124-36; WO2013/190272, WO2014/064424 for PolyTherics Ltd). dibromopyridazinediones (Maruani, A. et al. Nat. Commun. 2015, 6, 6645), galactosyl- and sialyltransferases (Zhou, Q. et al. Bioconjug. Chem. 2014, 25, 510-520; US Pat Appl 20140294867 for Sanofi-Genzyme), formylglycine generating enzyme (FGE) (Drake, P. M. et al. Bioconj. Chem. 2014, 25, 1331-41; Carrico, I. S. et al U.S. Pat. Nos. 7,985,783; 8,097,701; 8,349,910, and US Pat Appl 20140141025, 20100210543 for Redwood Bioscience), phosphopantetheinyl transferases (PPTases) (Grünewald, J. et al. Bioconjug. Chem. 2015, 26, 2554-62), sortase A (Beerli, R. R., et al. PLoS One 2015, 10, e0131177), genetically introduced glutamine tag with Streptoverticillium mobaraense transglutaminase (mTG) (Strop, P., Bioconj. Chem., 2014, 25, 855-62; Strop, P., et al., Chem. Biol. 2013, 20, 161-7; U.S. Pat. No. 8,871,908 for Rinat-Pfizer) or with microbial transglutaminase (MTGase) (Dennler, P., et al, 2014, Bioconjug. Chem. 25, 569-78; Siegmund, V. et al. Angew. Chemie—Int. Ed. 2015, 54, 13420-4; US pat appl 20130189287 for Innate Pharma; U.S. Pat. No. 7,893,019 for Bio-Ker S.r.l. (IT)), an enzyme/bacterium forming an isopeptide bond-peptide bonds that form outside of the protein main chain (Kang, H. J., et al. Science 2007, 318, 1625-8; Zakeri, B. et al. Proc. Natl. Acad. Sci. USA 2012, 109, E690-7; Zakeri, B. & Howarth, M. J. Am. Chem. Soc. 2010, 132, 4526-7).

We have disclosed several conjugation methods of rebridging a pair of thiols of the reduced inter chain disulfide bonds of a native antibody, such as using bromo maleimide and dibromomaleimide linkers (WO2014/009774), 2,3-disubstituted succinic/2-monosubstituted/2,3-disubstituted fumaric or maleic linkers (WO2015/155753, WO2016059622), acetylenedicarboxylic linkers (WO2015/151080, WO2016059622) or hydrazine linkers (WO2015/151081). The ADCs made with these linkers and methods have demonstrated better therapeutic index windows than the traditionally unselective conjugation via the cysteine or lysine residues on an antibody. Here we disclose the invention of bis-linkers and methods for conjugation of a cytotoxic molecule, particularly when the cytotoxic agent having dual groups of diamino, amino-hydroxyl, dihydroxyl, carboxyl, aldehyde and thiols. The immunoconjugates made with the bis-linkage have prolonged the half-life during the targeted delivery and minimized exposure to non-target cells, tissues or organs during the blood circulation, resulting in less the off-target toxicity.

### SUMMARY OF THE INVENTION

The present invention provides bis-linkage of an antibody with a cytotoxic agent, particularly when the cytotoxic agent having two functional groups of an amino, hydroxyl, diamino, amino-hydroxyl, dihydroxyl, carboxyl, hydrazine, or thiol. It also provides a bis-linker for conjugation of cell-binding molecule to a cytotoxic molecule in a specific manner.

In one aspect of the present invention, the bis-linkage is represented by Formula (I):

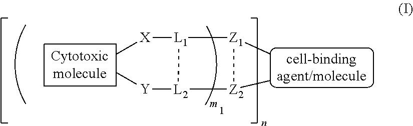

wherein

“ [symbol] ” represents a single bond;

“ [symbol] ” is optionally either a single bond, or a double bond, or can optionally be absent;

n and m₁ are 1 to 20 independently;

a cell-binding agent/molecule in the frame that links to Z₁ and Z₂ can be any kind presently known, or that become known, of a molecule that binds to, complexes with, or reacts with a moiety of a cell population sought to be therapeutically or otherwise biologically modified. Preferably the cell-binding agent/molecule is an immunotherapeutic protein, an antibody, an antibody fragment, or peptides having over four amino acids;

a cytotoxic molecule/agent in the frame is a therapeutic drug, or an immunotherapeutic protein/molecule, or a function molecule for enhancement of binding or stabilization of the cell-binding agent, or a cell-surface receptor binding ligand, or for inhibition of cell proliferation;

X and Y, represent the same or different, and independently, a functional group that links a cytotoxic drug via a disulfide, thioether, thioester, peptide, hydrazone, ether, ester, carbamate, carbonate, amine (secondary, tertiary, or quartary), imine, cycloheteroalkyane, heteroaromatic, alkoxime or amide bond; Preferably X and Y are independently selected from NH; NHNH; N(R₁); N(R₁)N(R₂); O; S; S—S, O—NH. O—N(R₁), CH₂—NH. CH₂—N(R₁), CH═NH. CH═N(R₁), S(O), S(O₂), P(O)(OH), S(O)NH, S(O₂)NH, P(O)(OH)NH, NHS(O)NH, NHS(O₂)NH, NHP(O)(OH)NH, N(R₁)S(O)N(R₂), N(R₁)S(O₂)N(R₂), N(R₁)P(O)(OH)N(R₂), OS(O)NH, OS(O₂)NH, OP(O)(OH)NH, C(O), C(NH), C(NR₁), C(O)NH, C(NH)NH, C(NR₁)NH, OC(O)NH, OC(NH)NH; OC(NR₁)NH, NHC(O)NH; NHC(NH)NH; NHC(NR₁)NH, C(O)NH, C(NH)NH, C(NR₁)NH, OC(O)N(R₁), OC(NH)N(R₁), OC(NR₁)N(R₁), NHC(O)N(R₁), NHC(NH)N(R₁), NHC(NR₁)N(R₁), N(R₁)C(O)N(R₁), N(R₁)C(NH)N(R₁), N(R₁)C(NR₁)N(R₁); or C₁-C₆ alkyl; C₂-C₈ alkenyl, heteroalkyl, alkylcycloalkyl, or heterocycloalkyl; C₃-C₈ aryl, Ar-alkyl, heterocyclic, carbocyclic, cycloalkyl, heteroalkylcycloalkyl, alkylcarbonyl, or heteroaryl;

Z₁ and Z₂ are, the same or different, and independently a function group that link to a cell-binding molecule, to form a disulfide, ether, ester, thioether, thioester, peptide, hydrazone, carbamate, carbonate, amine (secondary, tertiary, or quarter), imine, cycloheteroalkyane, heteroaromatic, alkyloxime or amide bond; Preferably Z₁ and Z₂ independently have the following structures: C(O)CH, C(O)C, C(O)CH₂, ArCH₂, C(O), NH; NHNH; N(R₁); N(R₁)N(R₂); O; S; S—S, O—NH. O—N(R₁), CH₂—NH. CH₂—N(R₁), CH═NH. CH═N(R₁), S(O), S(O₂), P(O)(OH), S(O)NH, S(O₂)NH, P(O)(OH)NH, NHS(O)NH, NHS(O₂)NH, NHP(O)(OH)NH, N(R₁)S(O)N(R₂), N(R₁)S(O₂)N(R₂), N(R₁)P(O)(OH)N(R₂), OS(O)NH, OS(O₂)NH, OP(O)(OH)NH, C(O), C(NH), C(NR₁), C(O)NH, C(NH)NH, C(NR₁)NH, OC(O)NH, OC(NH)NH; OC(NR₁)NH, NHC(O)NH; NHC(NH)NH; NHC(NR₁)NH, C(O)NH, C(NR₁)NH, OC(O)N(R₁), OC(NH)N(R₁), OC(NR₁)N(R₁), NHC(O)N(R₁), NHC(NH)N(R₁), NHC(NR₁)N(R₁), N(R₁)C(O)N(R₁), N(R₁)C(NH)N(R₁), N(R₁)C(NR₁)N(R₁); or C₁-C₈ alkyl, C₂-C₈ heteroalkyl, alkylcycloalkyl, heterocycloalkyl; C₃—C aryl, Ar-alkyl, heterocyclic, carbocyclic, cycloalkyl, heteroalkylcycloalkyl, alkylcarbonyl, heteroaryl;

Preferably Z₁ and Z₂ are linked to pairs of thiols of a cell-binding agent/molecule. The thiols are preferably pairs of sulfur atoms reduced from the inter chain disulfide bonds of the cell-binding agent by a reduction agent selected from dithiothreitol (DTT), dithioerythritol (DTE), L-glutathione (GSH), tris (2-carboxyethyl) phosphine (TCEP), 2-mercaptoethylamine (β-MEA), or/and beta mercaptoethanol (β-ME, 2-ME);

L₁ and L₂ are a chain of atoms selected from C, N, O, S, Si, and P, preferably having 0˜500 atoms, which covalently connects to X and Z₁, and Y and Z₂. The atoms used in forming the L₁ and L₂ may be combined in all chemically relevant ways, such as forming alkylene, alkenylene, and alkynylene, ethers, polyoxyalkylene, esters, amines, imines, polyamines, hydrazines, hydrazones, amides, ureas, semicarbazides, carbazides, alkoxyamines, alkoxylamines, urethanes, amino acids, peptides, acyloxylamines, hydroxamic acids, or combination above thereof. Preferably L₁ and L₂ are, the same or different, independently selected from O, NH, S, NHNH, N(R₃), N(R₃)N(R_{3′}), polyethyleneoxy unit of formula (OCH₂CH₂)ₚOR₃, or (OCH₂CH—(CH₃))ₚOR₃, or NH(CH₂CH₂O)ₚR₃, or NH(CH₂CH(CH₃)O)ₚR₃, or N[(CH₂CH₂O)ₚR₃]—[(CH₂CH₂O)_{p′}R_{3′}], or (OCH₂CH₂)ₚCOOR₃, or CH₂CH₂(OCH₂CH₂)ₚCOOR₃, wherein p and p′ are independently an integer selected from 0 to about 1000, or combination thereof; C₁-C₈ alkyl; C₂-C₈ heteroalkyl, or alkylcycloalkyl, heterocycloalkyl; C₃-C₈ aryl, Ar-alkyl, heterocyclic, carbocyclic, cycloalkyl, heteroalkylcycloalkyl, alkylcarbonyl, or heteroaryl;

wherein R₁, R₂, R₃, R₄, and R_{3′} are independently H; C₁-C₈ alkyl; C₂-C₈ heteroalkyl, alkylcycloalkyl, or heterocycloalkyl; C₃-C₈ aryl, Ar-alkyl, heterocyclic, carbocyclic, heteroalkylcycloalkyl, alkylcarbonyl, or heteroaryl; or C₁-C₈ carbon atoms esters, ether, or amide; or 1˜8 amino acids; or polyethyleneoxy having formula (OCH₂CH₂)ₚ or (OCH₂CH(CH₃))ₚ, wherein p is an integer from 0 to about 5000, or combination above thereof;

L₁ or L₂ may optionally be composed of one or more linker components of 6-maleimidocaproyl (“MC”), maleimidopropanoyl (“MP”), valine-citrulline (“val-cit” or “vc”), alanine-phenylalanine (“ala-phe” or “af”), p-aminobenzyloxycarbonyl (“PAB”), 4-thiopentanoate (“SPP”), 4-(N-maleimidomethyl)cyclohexane-1 carboxylate (“MCC”), (4-acetyl)amino-benzoate (“SAB”), 4-thio-butyrate (SPDB), 4-thio-2-hydroxysulfonyl-butyrate (2-Sulfo-SPDB), or natural or unnatural peptides having 1˜8 natural or unnatural amino acid unites. The natural aminoacid is preferably selected from aspartic acid, glutamic acid, arginine, histidine, lysine, serine, threonine, asparagine, glutamine, cysteine, selenocysteine, tyrosine, phenylalanine, glycine, proline, tryptophan, and alanine;

Additionally L₁ and L₂ may independently contain one of the following hydrophilic structures:

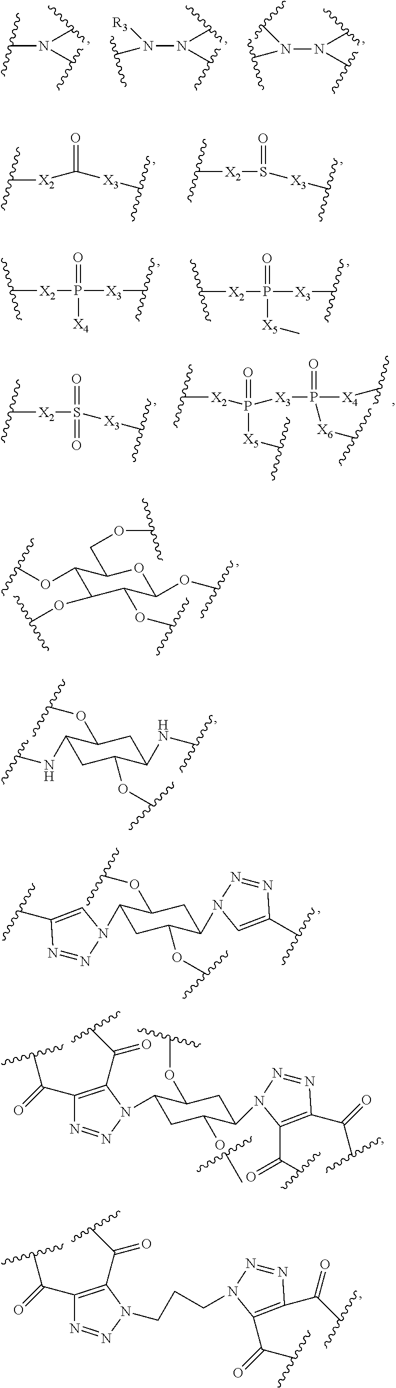

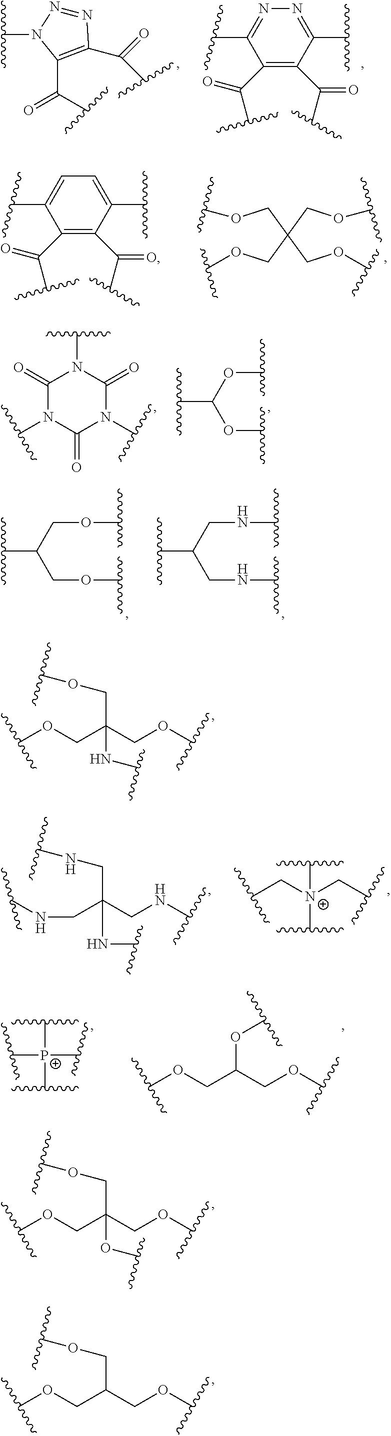

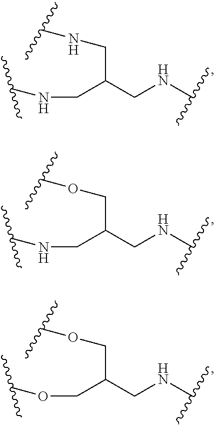

wherein [symbol] is the site of linkage; X₂, X₃, X₄, X₅, and X₆, are independently selected from NH; NHNH; N(R₃); N(R₃)N(R_{3′}); O; S; C₁-C₆ alkyl; C₂-C₆ heteroalkyl, alkylcycloalkyl, or heterocycloalkyl; C₃-C₈ aryl, Ar-alkyl, heterocyclic, carbocyclic, cycloalkyl, heteroalkylcycloalkyl, alkylcarbonyl, heteroaryl; or 1˜8 amino acids; Wherein R₃ and R_{3′} are independently H; C₁-C₈ alkyl; C₂-C₈ hetero-alkyl, alkylcycloalkyl, or heterocycloalkyl; C₃-C₈ aryl, Ar-alkyl, heterocyclic, carbocyclic, heteroalkylcycloalkyl, alkylcarbonyl, or heteroaryl; C₁-C₈ esters, ether, or amide; or polyethyleneoxy having formula (OCH₂CH₂)ₚ or (OCH₂CH(CH₃))ₚ, wherein p is an integer from 0 to about 5000, or combination above thereof;

X₁, and Y₁, is independently O, NH, CH₂, N(CH₃), NHNH, S, C(O)O, C(O)NH; m₁=1-20; In addition, L₁, L₂, X, Y Z₁, and Z₂ may be independently absent, but L₁ and Z₁, or L₂ and Z₂ may not be absent at the same time.

In another aspect, this invention provides a readily-reactive bis-linker of Formula (II) below, wherein two or more residues of the cell-binding molecule can simultaneously or sequentially react it to form Formula (I).

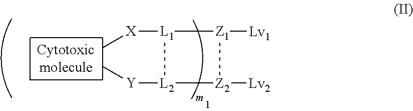

wherein:

“ [symbol] ” represents a single bond; “ [symbol] ” is optionally either a single bond, or a double bond, or a triple bond, or can optionally be absent;

It provided that when [symbol] represents a triple bond, both Lv₁ and Lv₂ are absent; Cytotoxic molecule in the frame, m₁, X, Y, L₁, L₂, Z₁, and Z₂ are defined the same as in Formula (I);

Lv₁ and Lv₂ represent the same or different leaving group that can be reacted with a thiol, amine, carboxylic acid, selenol, phenol or hydroxyl group on a cell-binding molecule. Such leaving groups are, but are not limited to, a halide (e.g., fluoride, chloride, bromide, and iodide), methanesulfonyl (mesyl), toluenesulfonyl (tosyl), trifluoromethyl-sulfonyl (triflate), trifluoromethylsulfonate, nitrophenoxyl, N-succinimidyloxyl (NHS), phenoxyl; dinitrophenoxyl; pentafluorophenoxyl, tetrafluorophenoxyl, trifluorophenoxyl, difluorophenoxyl, monofluorophenoxyl, pentachlorophenoxyl, 1H-imidazole-1-yl, chlorophenoxyl, dichlorophenoxyl, trichlorophenoxyl, tetrachlorophenoxyl, N-(benzotriazol-yl)oxyl, 2-ethyl-5-phenylisoxazolium-3′-sulfonyl, phenyloxadiazole-sulfonyl (-sulfone-ODA), 2-ethyl-5-phenylisoxazolium-yl, phenyloxadiazol-yl (ODA), oxadiazol-yl, unsaturated carbon (a double or a triple bond between carbon-carbon, carbon-nitrogen, carbon-sulfur, carbon-phosphorus, sulfur-nitrogen, phosphorus-nitrogen, oxygen-nitrogen, or carbon-oxygen), or an intermediate molecule generated with a condensation reagent for Mitsunobu reactions, or one of the following structure:

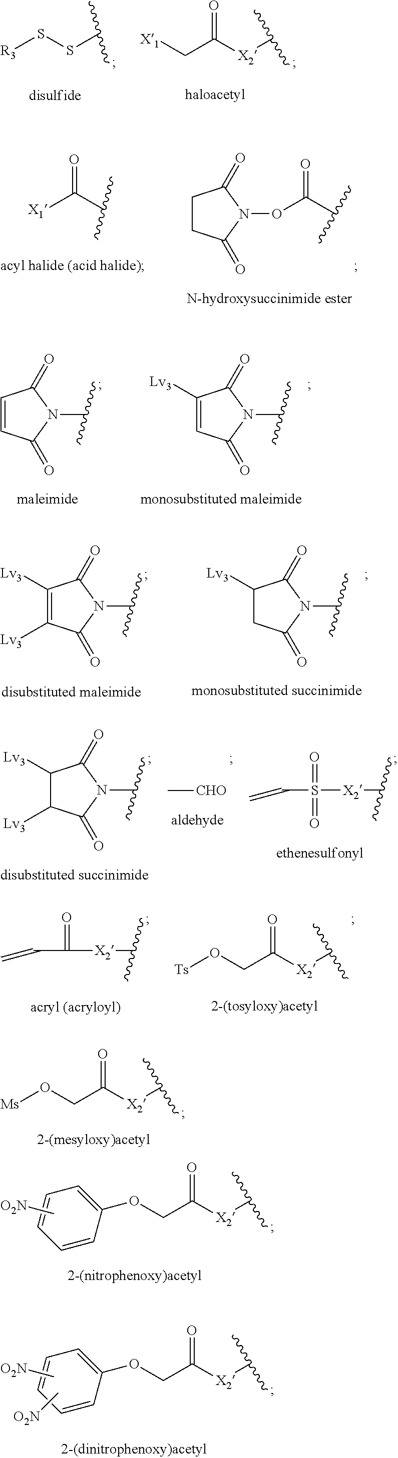

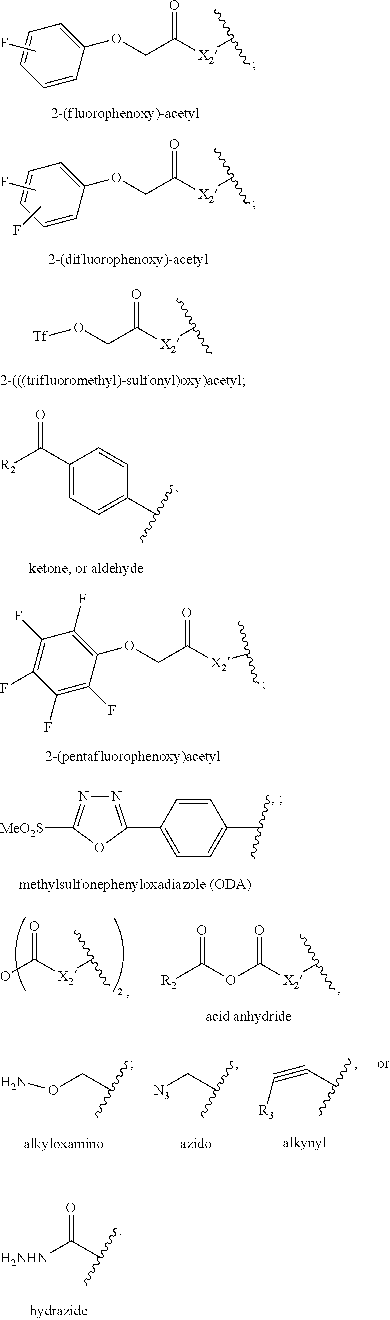

Wherein X₁′ is F, Cl, Br, I or Lv₃; X₂′ is O, NH, N(R₁), or CH₂; R₃ is independently H, aromatic, heteroaromatic, or aromatic group wherein one or several H atoms are replaced independently by —R₁, -halogen, —OR₁, —SR₁, —NR₁R₂, —NO₂, —S(O)R₁, —S(O)₂R₁, or —COOR₁; Lv₃ is a leaving group selected from F, Cl, Br, I, nitrophenol; N-hydroxysuccinimide (NHS); phenol; dinitrophenol; pentafluorophenol; tetrafluorophenol; difluorophenol; monofluorophenol; pentachlorophenol; triflate; imidazole; dichlorophenol; tetrachlorophenol; 1-hydroxybenzotriazole; tosylate; mesylate; 2-ethyl-5-phenylisoxazolium-3′-sulfonate, anhydrides formed its self, or formed with the other anhydride, e.g. acetyl anhydride, formyl anhydride; or an intermediate molecule generated with a condensation reagent for peptide coupling reactions or for Mitsunobu reactions;

R₁ and R₂ are independently selected from H, C₁-C₈ alkyl, C₂-C₈ alkenyl, heteroalkyl, alkylcycloalkyl, or heterocycloalkyl; C₃-C₈ aryl, Ar-alkyl, heterocyclic, carbocyclic, cycloalkyl, heteroalkylcycloalkyl, alkylcarbonyl, or heteroaryl, or C₂-C₈ esters, ether, or amide; or peptides containing 1-8 amino acids; or polyethyleneoxy having formula (OCH₂CH₂)ₚ or (OCH₂CH(CH₃))ₚ, wherein p is an integer from 0 to about 1000, or combination of above groups thereof.

In another aspect, this invention provides a readily-reactive bis-linker of Formula (III) of following, wherein two or more function groups of a cytotoxic molecule can react it simultaneously or sequentially to form Formula (I).

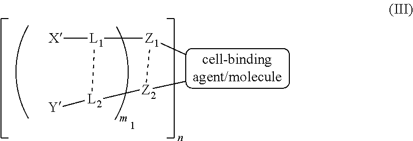

wherein:

m₁, n, cell-binding agent/molecule, L₁, L₂, Z₁, and Z₂ are defined the same as in Formula (I);

X′ and Y′ are a function group that can independently react with a residue groups of a cytotoxic drug simultaneously or sequentially to form X and Y respectively, wherein X and Y are defined in Formula (I);

X′ and Y′ are preferably N-hydroxysuccinimide esters, p-nitrophenyl esters, dinitrophenyl esters, pentafluorophenyl esters, pyridyldisulfides, nitropyridyldisulfides, maleimides, hydrazine, haloacetates, acetylenedicarboxylic group, carboxylic acid chlorides. Preferably X and Y have one of the following structures:

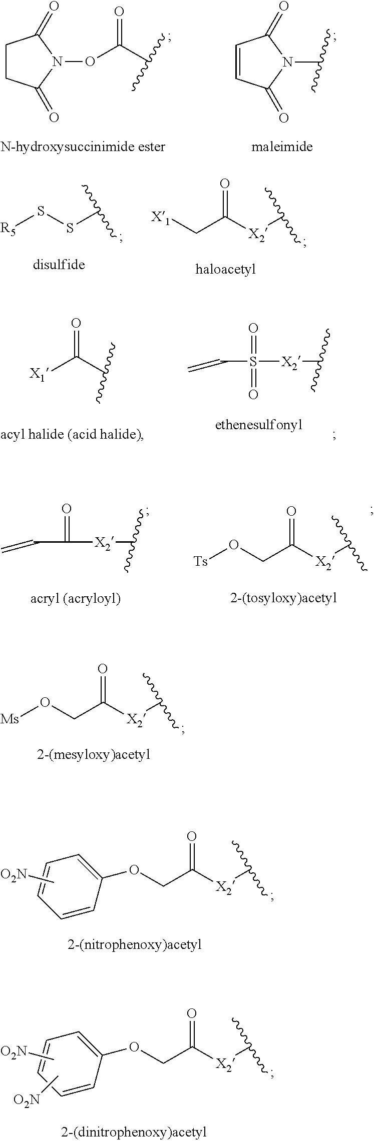

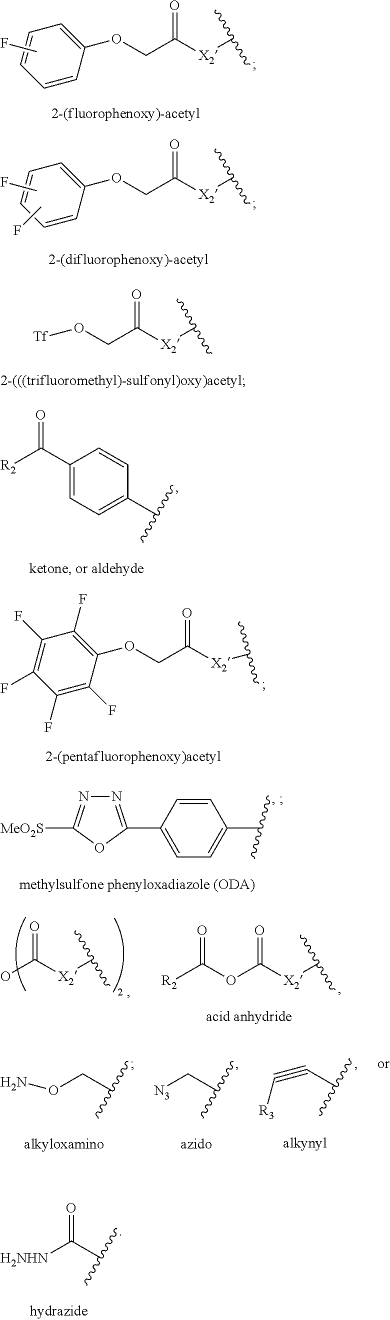

wherein X₁ is F, Cl, Br, I or Lv₃; X₂′ is O, NH, N(R₁), or CH₂; R₃ and R₅ are H, R₁, aromatic, heteroaromatic, or aromatic group wherein one or several H atoms are replaced independently by —R₁, -halogen, —OR₁, —SR₁, —NR₁R₂, —NO₂, —S(O)R₁, —S(O)₂R₁, or —COOR₁; Lv₃ is a leaving group selected from methanesulfonyl (mesyl), toluenesulfonyl (tosyl), trifluoromethyl-sulfonyl (triflate), trifluoromethylsulfonate, nitrophenoxyl, N-succinimidyloxyl (NHS), phenoxyl; dinitrophenoxyl; pentafluorophenoxyl, tetrafluoro-phenoxyl, trifluorophenoxyl, difluorophenoxyl, monofluoro-phenoxyl, pentachlorophenoxyl, 1H-imidazole-1-yl, chlorophenoxyl, dichlorophenoxyl, trichlorophenoxyl, tetrachlorophenoxyl, N-(benzotriazol-yl)oxyl, 2-ethyl-5-phenylisoxazolium-yl, phenyloxadiazol-yl (ODA), oxadiazol-yl, or an intermediate molecule generated with a condensation reagent for Mitsunobu reactions, wherein R₁ and R₂ are defined above.

In another aspect, this invention provides a readily-reactive bis-linker of Formula (IV) below, wherein a cytotoxic molecule and a cell-binding molecule can react it independently, or simultaneously, or sequentially to form Formula (I).

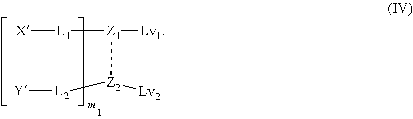

wherein m₁, L₁, L₂, Z₁, and Z₂ are defined the same as in Formula (I); Lv₁ and Lv₂ are defined in Formula (II), and X′ and Y′ are defined in Formula (III);

n is 1˜20; and T are described the same previously in Formula (I).

The present invention further relates to a method of making a cell-binding molecule-drug conjugate of Formula (I).

### BRIEF DESCRIPTION OF THE DRAWINGS

FIG. 1 shows the general synthesis of bis-linked conjugates of the patent application through dual linkage of a phenyl diamine, a phenyl diol, or an aminophenol group of a drug at one end, and a pair of thiols in a cell-binding molecule at the other end, wherein the wavy line is the rest part of a drug or a linked component of a drug which is absent (not shown here).

FIG. 2 shows the synthesis of analogs of tyrosine (Tyr) and tubutyrosine (Tut) that have an amino or nitro group on the benzene ring for bis-linked to a cell-binding molecule.

FIG. 3 shows the synthesis of components of tubulysin analogs.

FIG. 4 shows the synthesis of components of tubulysin analogs.

FIG. 5 shows the synthesis of a tubulysin analog containing a bis-linker and its conjugation to an antibody via a pair of thiols in the antibody.

FIG. 6 shows the synthesis of tubulysin analogs containing a bis-linker and their conjugations to an antibody via a pair of thiols in the antibody.

FIG. 7 shows the synthesis of tubulysin analogs containing a bis-linker and their conjugations to an antibody via a pair of thiols in the antibody.

FIG. 8 shows the synthesis of tubulysin analogs containing a bis-linker and their conjugations to an antibody via a pair of thiols in the antibody.

FIG. 9 shows the synthesis of tubulysin analogs containing a bis-linker and their conjugations to an antibody via a pair of thiols in the antibody.

FIG. 10 shows the synthesis of tubulysin analogs containing a bis-linker and their conjugations to an antibody via a pair of thiols in the antibody.

FIG. 11 shows the synthesis of tubulysin analogs containing a bis-linker and their conjugations to an antibody via a pair of thiols in the antibody.

FIG. 12 shows the synthesis of components of bis-linkers and a bis-linkage to a tubutyrosine (Tup) analog, a component of tubulysin.

FIG. 13 shows the synthesis of tubulysin analogs containing a bis-linker and their conjugations to an antibody via a pair of thiols in the antibody.

FIG. 14 shows the synthesis of a tubulysin analog containing a bis-linker and its conjugation to an antibody via a pair of thiols in the antibody.

FIG. 15 shows the synthesis of a tubulysin analog containing a bis-linker and its conjugation to an antibody via a pair of thiols in the antibody.

FIG. 16 shows the synthesis of a tubulysin analog containing a bis-linker and its conjugation to an antibody via a pair of thiols in the antibody.

FIG. 17 shows the synthesis of conjugation of tubulysin analog containing a bis-linker to an antibody via a pair of thiols on the antibody, and the synthesis of a tubuphenylalaine (Tup) analog having a bis-linker with dual amide linkage.

FIG. 18 shows the synthesis of a tubulysin analog containing a bis-linker and its conjugation to an antibody via a pair of thiols in the antibody.

FIG. 19 shows the synthesis of conjugation of tubulysin analog containing a bis-linker to an antibody via a pair of thiols in an antibody, and the synthesis of a tubuphenylalaine (Tup) analog having a bis-linker with dual amide linkage.

FIG. 20 shows the synthesis of a tubulysin analog containing a bis-linker and its conjugation to an antibody via a pair of thiols in the antibody.

FIG. 21 shows the synthesis of a tubulysin analog containing a bis-linker and its conjugation to an antibody via a pair of thiols in the antibody.

FIG. 22 shows the synthesis of a component of dimethyl auristatin analog.

FIG. 23 shows the synthesis of dimethyl auristatin F analogs containing a bis-linker and their conjugation to an antibody via a pair of thiols in the antibody.

FIG. 24 shows the synthesis of dimethyl auristatin F analogs containing a bis-linker and their conjugation to an antibody via a pair of thiols in the antibody.

FIG. 25 shows the synthesis of dimethyl auristatin F analogs containing a bis-linker and their conjugation to an antibody via a pair of thiols in the antibody.

FIG. 26 shows the synthesis of dimethyl auristatin F analogs containing a bis-linker and their conjugation to an antibody via a pair of thiols in the antibody.

FIG. 27 shows the synthesis of dimethyl auristatin F analogs containing a bis-linker and their conjugation to an antibody via a pair of thiols in the antibody.

FIG. 28 shows the synthesis of dimethyl auristatin F analogs containing a bis-linker and their conjugation to an antibody via a pair of thiols in the antibody.

FIG. 29 shows the synthesis of an amatoxin analog having a diamino group on its aromatic ring.

FIG. 30 shows the synthesis of an amatoxin analog containing a bis-linker and its conjugation to an antibody via a pair of thiols in the antibody.

FIG. 31 shows the synthesis of a bis-linker and its linkage to an amatoxin analog.

FIG. 32 shows the synthesis of amatoxin analogs containing a bis-linker and their conjugation to an antibody via a pair of thiols in the antibody.

FIG. 33 shows the synthesis of amatoxin analogs containing a bis-linker and their conjugation to an antibody via a pair of thiols in the antibody.

FIG. 34 shows the synthesis of amatoxin analogs containing a bis-linker and their conjugation to an antibody via a pair of thiols in the antibody.

FIG. 35 shows the synthesis of amatoxin analogs and dimethyl auristatin F analogs containing a bis-linker and their conjugation to an antibody via a pair of thiols on an antibody.

FIG. 36 shows the synthesis of tubulysin analogs and CBI dimer analogs containing a bis-linker and their conjugation to an antibody via a pair of thiols in the antibody.

FIG. 37 shows the synthesis of CBI dimer analogs containing a bis-linker and their conjugation to an antibody via a pair of thiols in the antibody.

FIG. 38 shows the synthesis of CBI dimer analogs containing a bis-linker and their conjugation to an antibody via a pair of thiols in the antibody.

FIG. 39 shows the synthesis of CBI dimer analogs containing a bis-linker and their conjugation to an antibody via a pair of thiols in the antibody.

FIG. 40 shows the synthesis of CBI dimer analogs containing a bis-linker and their conjugation to an antibody via a pair of thiols in the antibody.

FIG. 41 shows the synthesis of PBD dimer analogs containing a bis-linker.

FIG. 42 shows the synthesis of PBD dimer analogs containing a bis-linker and their conjugation to an antibody via a pair of thiols in the antibody.

FIG. 43 shows the synthesis of PBD dimer analogs containing a bis-linker and their conjugation to an antibody via a pair of thiols in the antibody.

FIG. 44 shows the synthesis of PBD dimer analogs containing a bis-linker and their conjugation to an antibody via a pair of thiols in the antibody.

FIG. 45 shows the synthesis of PBD dimer analogs containing a bis-linker and their conjugation to an antibody via a pair of thiols in the antibody.

FIG. 46 shows the synthesis of PBD dimer analogs containing a bis-linker and their conjugation to an antibody via a pair of thiols in the antibody.

FIG. 47 shows the comparison of the anti-tumor effect of conjugate compounds A-3a, B-6a, B-12a, B-15a, B-18a, B-20a, B-21a, B-24a, B-28a, C-3a, D-2a along with T-DM1 and PBS (control) using human gastric tumor N87 cell model, i.v., one injection at dosing of 3 mg/kg for conjugates A-3a, B-6a, B-12a, B-15a, B-18a, B-20a, B-21a, B-24a, B-28a, T-DM1 and at dosing of 1 mg/kg for conjugates C-3a and D-1a. All 12 conjugates tested here demonstrated anti-tumor activity. Animals at the groups of conjugate compounds B-24a, C-3a, B-20a, B-21a and D-20a demonstrated better anti-tumor activity than T-DM1. However, the animals at the groups of conjugate compounds B-18a, B-15a, A-3a, B-6a, B-28a and B-12a showed worse anti-tumor activity than T-DM1. T-DM1 at dose of 3 mg/Kg inhibited the tumor growth for 28 days but it was not able to eliminate the tumors at any time during the test. In contrast, conjugate compounds B-20a, B-21a, and D-20a eradicate some animal's tumors from day 15 until day 43.

FIG. 48 shows the pictures of the in vivo tested animals alone with their peeled tumors of the groups of PBS, conjugates A-3a, B-15a, B-21a, and T-DM1 after the animals were sacrificed. Five of eight animals of the group of conjugate B-21a had no tumor found (labeled as [symbol] )). Five of eight animals of the group of conjugate B-15a died (labeled as [symbol] ) at day 43 due to its tumor was too big.

FIG. 49 shows stability study of conjugate B-21a in the mouse serum in comparison with regular mono-linked conjugate T-1a and T-DM1. It indicates that the conjugate having the bis-linkage is more stable than the regular conjugates containing mono-linkage in the mouse serum.

### DETAILED DESCRIPTION OF THE INVENTION

### Definitions

“Alkyl” refers to an aliphatic hydrocarbon group or univalent groups derived from alkane by removal of one or two hydrogen atoms from carbon atoms. It may be straight or branched having C₁-C₈ (1 to 8 carbon atoms) in the chain. “Branched” means that one or more lower C numbers of alkyl groups such as methyl, ethyl or propyl are attached to a linear alkyl chain. Exemplary alkyl groups include methyl, ethyl, n-propyl, i-propyl, n-butyl, t-butyl, n-pentyl, 3-pentyl, octyl, nonyl, decyl, cyclopentyl, cyclohexyl, 2,2-dimethylbutyl, 2,3-dimethylbutyl, 2,2-dimethylpentyl, 2,3-dimethylpentyl, 3,3-dimethylpentyl, 2,3,4-trimethylpentyl, 3-methyl-hexyl, 2,2-dimethylhexyl, 2,4-dimethylhexyl, 2,5-dimethylhexyl, 3,5-dimethylhexyl, 2,4-dimethylpentyl, 2-methylheptyl, 3-methylheptyl, n-heptyl, isoheptyl, n-octyl, and isooctyl. A C₁-C₈ alkyl group can be unsubstituted or substituted with one or more groups including, but not limited to, —C₁-C₈ alkyl, —O—(C₁-C₈ alkyl), -aryl, —C(O)R′, —OC(O)R′, —C(O)OR′, —C(O)NH₂, —C(O)NHR′, —C(O)N(R′)₂, —NHC(O)R′, —SR′, —S(O)₂R′, —S(O)R′, —OH, -halogen, —N₃, —NH₂, —NH(R′), —N(R′)₂ and —CN; where each R′ is independently selected from —C₁-C₈ alkyl and aryl.

“Halogen” refers to fluorine, chlorine, bromine or iodine atom; preferably fluorine and chlorine atom.

“Heteroalkyl” refers to C₂-C₈ alkyl in which one to four carbon atoms are independently replaced with a heteroatom from the group consisting of O, S and N.

“Carbocycle” refers to a saturated or unsaturated ring having 3 to 8 carbon atoms as a monocycle or 7 to 13 carbon atoms as a bicycle. Monocyclic carbocycles have 3 to 6 ring atoms, more typically 5 or 6 ring atoms. Bicyclic carbocycles have 7 to 12 ring atoms, arranged as a bicycle [4,5], [5,5], [5,6] or [6,6] system, or 9 or 10 ring atoms arranged as a bicycle [5,6] or [6,6] system. Representative C₃-C₈ carbocycles include, but are not limited to, -cyclopropyl, -cyclobutyl, -cyclopentyl, -cyclopentadienyl, -cyclohexyl, -cyclohexenyl, -1,3-cyclohexadienyl, -1,4-cyclohexadienyl, -cycloheptyl, -1,3-cycloheptadienyl, -1,3,5-cycloheptatrienyl, -cyclooctyl, and -cyclooctadienyl.

A “C₃-C₈ carbocycle” refers to a 3-, 4-, 5-, 6-, 7- or 8-membered saturated or unsaturated nonaromatic carbocyclic ring. A C₃-C₈ carbocycle group can be unsubstituted or substituted with one or more groups including, but not limited to, —C₁-C₈ alkyl, —O—(C₁-C₈ alkyl), -aryl, —C(O)R′, —OC(O)R′, —C(O)OR′, —C(O)NH₂, —C(O)NHR′, —C(O)N(R′)₂, —NHC(O)R′, —SR′, —S(O)R′, —S(O)₂R′, —OH, -halogen, —N₃, —NH₂, —NH(R′), —N(R′)₂ and —CN; where each R′ is independently selected from —C₁-C₈ alkyl and aryl.

“Alkenyl” refers to an aliphatic hydrocarbon group containing a carbon-carbon double bond which may be straight or branched having 2 to 8 carbon atoms in the chain. Exemplary alkenyl groups include ethenyl, propenyl, n-butenyl, i-butenyl, 3-methylbut-2-enyl, n-pentenyl, hexylenyl, heptenyl, octenyl.

“Alkynyl” refers to an aliphatic hydrocarbon group containing a carbon-carbon triple bond which may be straight or branched having 2 to 8 carbon atoms in the chain. Exemplary alkynyl groups include ethynyl, propynyl, n-butynyl, 2-butynyl, 3-methylbutynyl, 5-pentynyl, n-pentynyl, hexylynyl, heptynyl, and octynyl.

“Alkylene” refers to a saturated, branched or straight chain or cyclic hydrocarbon radical of 1-18 carbon atoms, and having two monovalent radical centers derived by the removal of two hydrogen atoms from the same or two different carbon atoms of a parent alkane. Typical alkylene radicals include, but are not limited to: methylene (—CH₂—), 1,2-ethyl (—CH₂CH₂—), 1,3-propyl (—CH₂CH₂CH₂—), 1,4-butyl (—CH₂CH₂CH₂CH₂—), and the like.

“Alkenylene” refers to an unsaturated, branched or straight chain or cyclic hydrocarbon radical of 2-18 carbon atoms, and having two monovalent radical centers derived by the removal of two hydrogen atoms from the same or two different carbon atoms of a parent alkene. Typical alkenylene radicals include, but are not limited to: 1,2-ethylene (—CH═CH—).

“Alkynylene” refers to an unsaturated, branched or straight chain or cyclic hydrocarbon radical of 2-18 carbon atoms, and having two monovalent radical centers derived by the removal of two hydrogen atoms from the same or two different carbon atoms of a parent alkyne. Typical alkynylene radicals include, but are not limited to: acetylene, propargyl and 4-pentynyl.

“Aryl” or “Ar” refers to an aromatic or hetero aromatic group, composed of one or several rings, comprising three to fourteen carbon atoms, preferentially six to ten carbon atoms. The term of “hetero aromatic group” refers one or several carbon on aromatic group, preferentially one, two, three or four carbon atoms are replaced by O, N, Si, Se, P or S, preferentially by O, S, and N. The term aryl or Ar also refers to an aromatic group, wherein one or several H atoms are replaced independently by —R′, -halogen, —OR′, or —SR′, —NR′R″, —N═NR′, —N═R′, —NR′R″, —NO₂, —S(O)R′, —S(O)₂R′, —S(O)₂OR′, —OS(O)₂OR′, —PR′R″, —P(O)R′R″, —P(OR′)(OR″), —P(O)(OR′)(OR″) or —OP(O)(OR′)(OR″) wherein R′, R″ are independently H, alkyl, alkenyl, alkynyl, heteroalkyl, aryl, arylalkyl, carbonyl, or pharmaceutical salts.

“Heterocycle” refers to a ring system in which one to four of the ring carbon atoms are independently replaced with a heteroatom from the group of O, N, S, Se, B, Si and P. Preferable heteroatoms are O, N and S. Heterocycles are also described in The Handbook of Chemistry and Physics, 78th Edition, CRC Press, Inc., 1997-1998, p. 225 to 226, the disclosure of which is hereby incorporated by reference. Preferred nonaromatic heterocyclic include epoxy, aziridinyl, thiiranyl, pyrrolidinyl, pyrazolidinyl, imidazolidinyl, oxiranyl, tetrahydrofuranyl, dioxolanyl, tetrahydropyranyl, dioxanyl, dioxolanyl, piperidyl, piperazinyl, morpholinyl, pyranyl, imidazolinyl, pyrrolinyl, pyrazolinyl, thiazolidinyl, tetrahydrothiopyranyl, dithianyl, thiomorpholinyl, dihydropyranyl, tetrahydropyranyl, dihydropyranyl, tetrahydropyridyl, dihydropyridyl, tetrahydropyrimidinyl, dihydrothiopyranyl, azepanyl, as well as the fused systems resulting from the condensation with a phenyl group.

The term “heteroaryl” or aromatic heterocycles refers to a 3 to 14, preferably 5 to 10 membered aromatic hetero, mono-, bi-, or multi-cyclic ring. Examples include pyrrolyl, pyridyl, pyrazolyl, thienyl, pyrimidinyl, pyrazinyl, tetrazolyl, indolyl, quinolinyl, purinyl, imidazolyl, thienyl, thiazolyl, benzothiazolyl, furanyl, benzofuranyl, 1,2,4-thiadiazolyl, isothiazolyl, triazolyl, tetrazolyl, isoquinolyl, benzothienyl, isobenzofuryl, pyrazolyl, carbazolyl, benzimidazolyl, isoxazolyl, pyridyl-N-oxide, as well as the fused systems resulting from the condensation with a phenyl group.

“Alkyl”, “cycloalkyl”, “alkenyl”, “alkynyl”, “aryl”, “heteroaryl”, “heterocyclic” and the like refer also to the corresponding “alkylene”, “cycloalkylene”, “alkenylene”, “alkynylene”, “arylene”, “heteroarylene”, “heterocyclene” and the likes which are formed by the removal of two hydrogen atoms.

“Arylalkyl” refers to an acyclic alkyl radical in which one of the hydrogen atoms bonded to a carbon atom, typically a terminal or sp³ carbon atom, is replaced with an aryl radical. Typical arylalkyl groups include, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like.

“Heteroarylalkyl” refers to an acyclic alkyl radical in which one of the hydrogen atoms bonded to a carbon atom, typically a terminal or sp³ carbon atom, is replaced with a heteroaryl radical. Examples of heteroarylalkyl groups are 2-benzimidazolylmethyl, 2-furylethyl.

Examples of a “hydroxyl protecting group” include, methoxymethyl ether, 2-methoxyethoxymethyl ether, tetrahydropyranyl ether, benzyl ether, p-methoxybenzyl ether, trimethylsilyl ether, triethylsilyl ether, triisopropylsilyl ether, t-butyldimethylsilyl ether, triphenylmethylsilyl ether, acetate ester, substituted acetate esters, pivaloate, benzoate, methanesulfonate and p-toluenesulfonate.

“Leaving group” refers to a functional group that can be substituted by another functional group. Such leaving groups are well known in the art, and examples include, a halide (e.g., chloride, bromide, and iodide), methanesulfonyl (mesyl), p-toluenesulfonyl (tosyl), trifluoromethylsulfonyl (triflate), and trifluoromethylsulfonate. A preferred leaving group is selected from nitrophenol; N-hydroxysuccinimide (NHS); phenol; dinitrophenol; pentafluorophenol; tetrafluorophenol; difluorophenol; monofluorophenol; pentachlorophenol; triflate; imidazole; dichlorophenol; tetrachlorophenol; 1-hydroxybenzotriazole; tosylate; mesylate; 2-ethyl-5-phenylisoxazolium-3′-sulfonate, anhydrides formed its self, or formed with the other anhydride, e.g. acetyl anhydride, formyl anhydride; or an intermediate molecule generated with a condensation reagent for peptide coupling reactions or for Mitsunobu reactions.

The following abbreviations may be used herein and have the indicated definitions: Boc, tert-butoxy carbonyl; BroP, bromotrispyrrolidinophosphonium hexafluorophosphate; CDI, 1,1′-carbonyldiimidazole; DCC, dicyclohexylcarbodiimide; DCE, dichloroethane; DCM, dichloromethane; DIAD, diisopropylazodicarboxylate; DIBAL-H, diisobutyl-aluminium hydride; DIPEA, diisopropylethylamine; DEPC, diethyl phosphorocyanidate; DMA, N,N-dimethyl acetamide; DMAP, 4-(N, N-dimethylamino)pyridine; DMF, N,N-dimethylformamide; DMSO, dimethylsulfoxide; DTT, dithiothreitol; EDC, 1-(3-dimethylaminopropyl)-3-ethylcarbodiimide hydrochloride; ESI-MS, electrospray mass spectrometry; HATU, O-(7-azabenzotriazol-1-yl)-N, N, N′, N′-tetramethyluronium hexafluorophosphate; HOBt, 1-hydroxybenzotriazole; HPLC, high pressure liquid chromatography; NHS, N-Hydroxysuccinimide; MMP, 4-methylmorpholine; PAB, p-aminobenzyl; PBS, phosphate-buffered saline (pH 7.0˜7.5); PEG, polyethylene glycol; SEC, size-exclusion chromatography; TCEP, tris(2-carboxyethyl)phosphine; TFA, trifluoroacetic acid; THF, tetrahydrofuran; Val, valine.

The “amino acid(s)” can be natural and/or unnatural amino acids, preferably alpha-amino acids. Natural amino acids are those encoded by the genetic code, which are alanine, arginine, asparagine, aspartic acid, cysteine, glutamic acid, glutamine, glycine, histidine, isoleucine, leucine, lysine, methionine, phenylalanine, proline, serine, threonine, tyrosine. tryptophan and valine. The unnatural amino acids are derived forms of proteinogenic amino acids. Examples include hydroxyproline, lanthionine, 2-aminoisobutyric acid, dehydroalanine, gamma-aminobutyric acid (the neurotransmitter), ornithine, citrulline, beta alanine (3-aminopropanoic acid), gamma-carboxyglutamate, selenocysteine (present in many noneukaryotes as well as most eukaryotes, but not coded directly by DNA), pyrrolysine (found only in some archaea and one bacterium), N-formylmethionine (which is often the initial amino acid of proteins in bacteria, mitochondria, and chloroplasts), 5-hydroxytryptophan, L-dihydroxyphenylalanine, triiodothyronine, L-3,4-dihydroxyphenylalanine (DOPA), and O-phosphoserine. The term amino acid also includes amino acid analogs and mimetics. Analogs are compounds having the same general H₂N(R)CHCO₂H structure of a natural amino acid, except that the R group is not one found among the natural amino acids. Examples of analogs include homoserine, norleucine, methionine-sulfoxide, and methionine methyl sulfonium. Preferably, an amino acid mimetic is a compound that has a structure different from the general chemical structure of an alpha-amino acid but functions in a manner similar to one. The term “unnatural amino acid” is intended to represent the “D” stereochemical form, the natural amino acids being of the “L” form. When 1˜8 amino acids are used in this patent application, amino acid sequence is then preferably a cleavage recognition sequence for a protease. Many cleavage recognition sequences are known in the art. See, e.g., Matayoshi et al. Science 247: 954 (1990); Dunn et al. Meth. Enzymol. 241: 254 (1994); Seidah et al. Meth. Enzymol. 244: 175 (1994); Thornberry, Meth. Enzymol. 244: 615 (1994); Weber et al. Meth. Enzymol. 244: 595 (1994); Smith et al. Meth. Enzymol. 244: 412 (1994); and Bouvier et al. Meth. Enzymol. 248: 614 (1995); the disclosures of which are incorporated herein by reference. In particular, the sequence is selected from the group consisting of Val-Cit, Ala-Val, Ala-Ala, Val-Val, Val-Ala-Val, Lys-Lys, Ala-Asn-Val, Val-Leu-Lys, Cit-Cit, Val-Lys, Ala-Ala-Asn, Lys, Cit, Ser, and Glu.

The “glycoside” is a molecule in which a sugar group is bonded through its anomeric carbon to another group via a glycosidic bond. Glycosides can be linked by an O- (an O-glycoside), N- (a glycosylamine), S- (a thioglycoside), or C- (a C-glycoside) glycosidic bond. Its core the empirical formula is Cₘ(H₂O)ₙ (where m could be different from n, and m and n are <36), Glycoside herein includes glucose (dextrose), fructose (levulose) allose, altrose, mannose, gulose, iodose, galactose, talose, galactosamine, glucosamine, sialic acid, N-acetylglucosamine, sulfoquinovose (6-deoxy-6-sulfo-D-glucopyranose), ribose, arabinose, xylose, lyxose, sorbitol, mannitol, sucrose, lactose, maltose, trehalose, maltodextrins, raffinose, Glucuronic acid (glucuronide), and stachyose. It can be in D form or L form, 5 atoms cyclic furanose forms, 6 atoms cyclic pyranose forms, or acyclic form, α-isomer (the —OH of the anomeric carbon below the plane of the carbon atoms of Haworth projection), or a β-isomer (the —OH of the anomeric carbon above the plane of Haworth projection). It is used herein as a monosaccharide, disaccharide, polyols, or oligosaccharides containing 3-6 sugar units.

“Pharmaceutically” or “pharmaceutically acceptable” refer to molecular entities and compositions that do not produce an adverse, allergic or other untoward reaction when administered to an animal, or a human, as appropriate.

“Pharmaceutically acceptable solvate” or “solvate” refer to an association of one or more solvent molecules and a disclosed compound. Examples of solvents that form pharmaceutically acceptable solvates include, but are not limited to, water, isopropanol, ethanol, methanol, DMSO, ethyl acetate, acetic acid and ethanolamine.

“Pharmaceutically acceptable excipient” includes any carriers, diluents, adjuvants, or vehicles, such as preserving or antioxidant agents, fillers, disintegrating agents, wetting agents, emulsifying agents, suspending agents, solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions as suitable therapeutic combinations.

As used herein, “pharmaceutical salts” refer to derivatives of the disclosed compounds wherein the parent compound is modified by making acid or base salts thereof. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, tartaric, citric, methanesulfonic, benzenesulfonic, glucuronic, glutamic, benzoic, salicylic, toluenesulfonic, oxalic, fumaric, maleic, lactic and the like. Further addition salts include ammonium salts such as tromethamine, meglumine, epolamine, etc., metal salts such as sodium, potassium, calcium, zinc or magnesium.

The pharmaceutical salts of the present invention can be synthesized from the parent compound which contains a basic or acidic moiety by conventional chemical methods. Generally, such salts can be prepared via reaction the free acidic or basic forms of these compounds with a stoichiometric amount of the appropriate base or acid in water or in an organic solvent, or in a mixture of the two. Generally, non-aqueous media like ether, ethyl acetate, ethanol, isopropanol, or acetonitrile are preferred. Lists of suitable salts are found in Remington's Pharmaceutical Sciences, 17^{th} ed., Mack Publishing Company, Easton, Pa., 1985, p. 1418, the disclosure of which is hereby incorporated by reference.

“Administering” or “administration” refers to any mode of transferring, delivering, introducing or transporting a pharmaceutical drug or other agent to a subject. Such modes include oral administration, topical contact, intravenous, intraperitoneal, intramuscular, intralesional, intranasal, subcutaneous or intrathecal administration. Also contemplated by the present invention is utilization of a device or instrument in administering an agent. Such device may utilize active or passive transport and may be slow-release or fast-release delivery device.

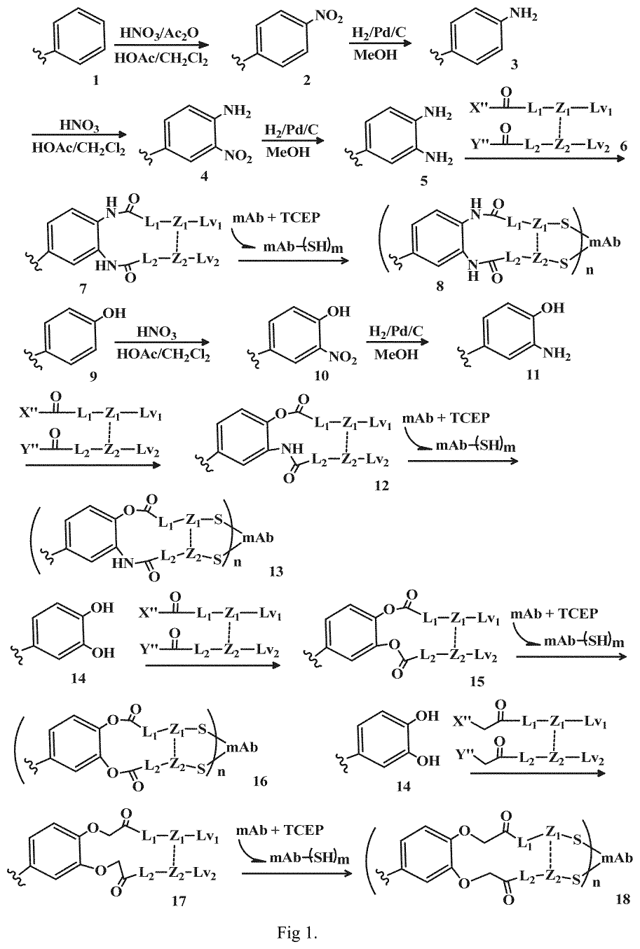

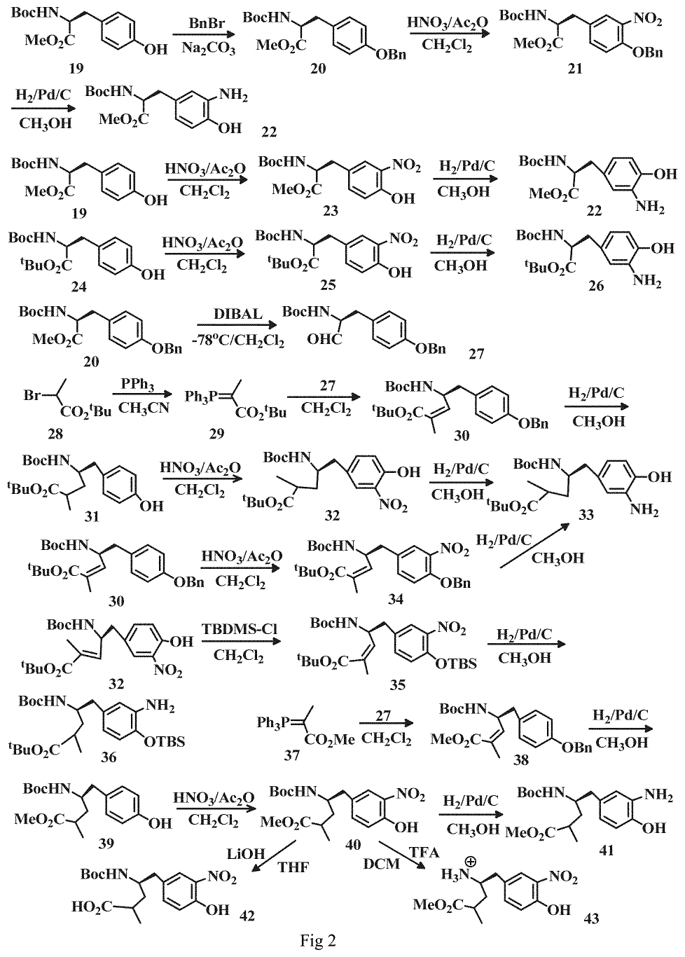

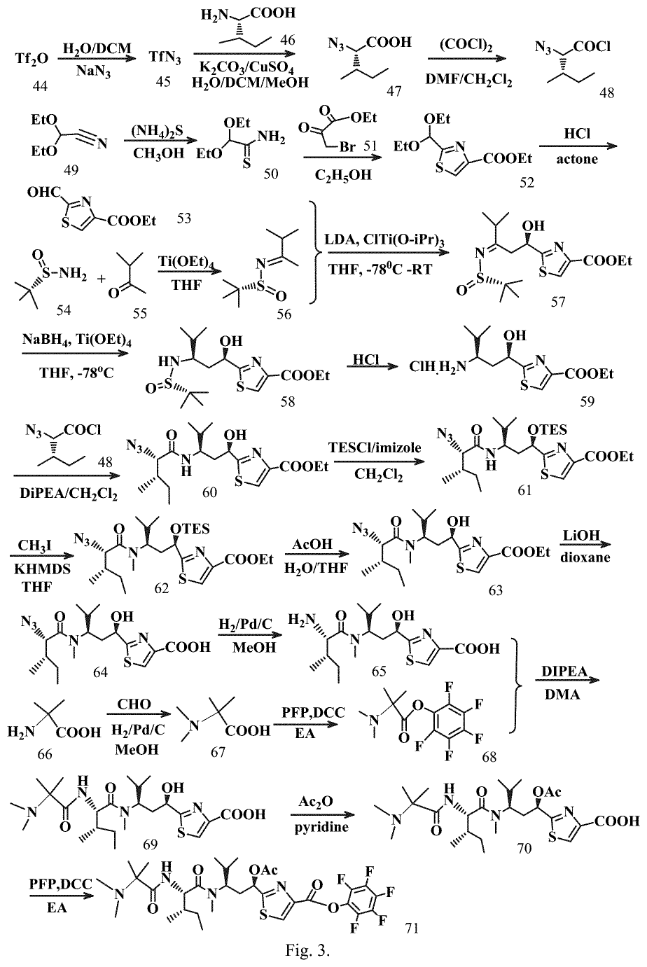

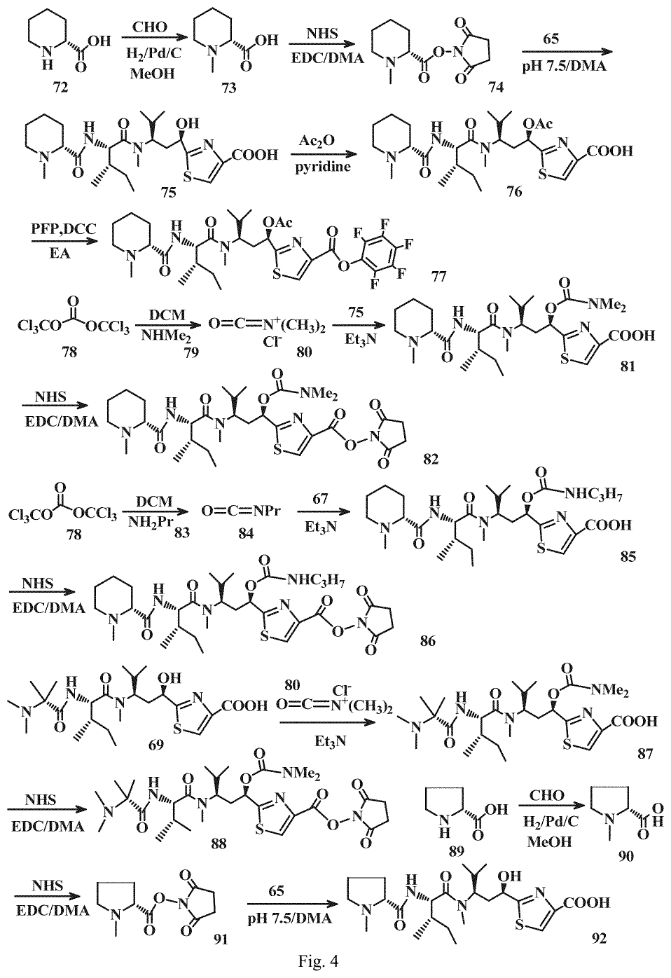

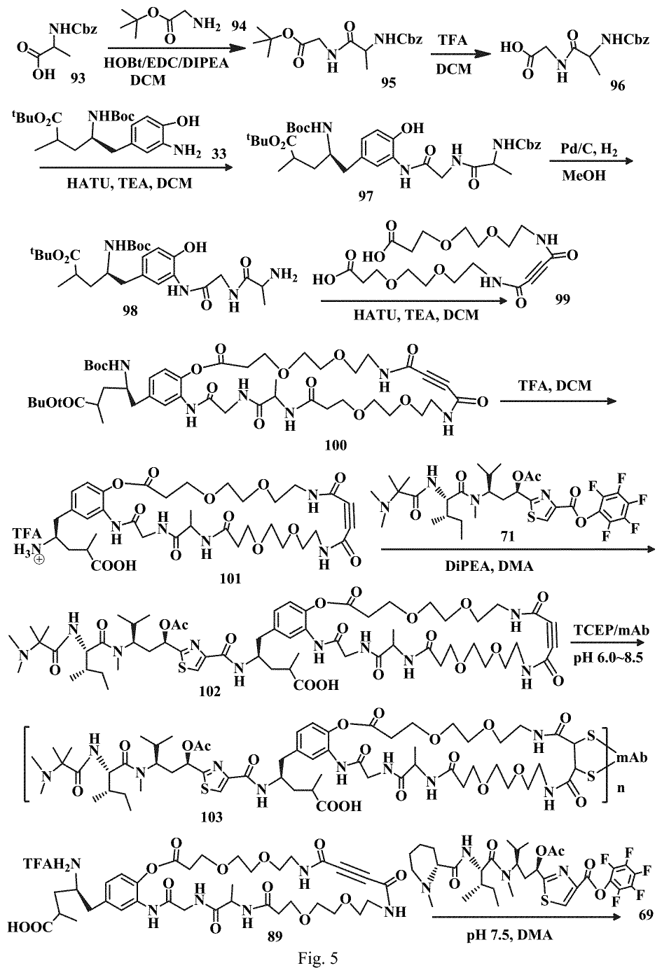

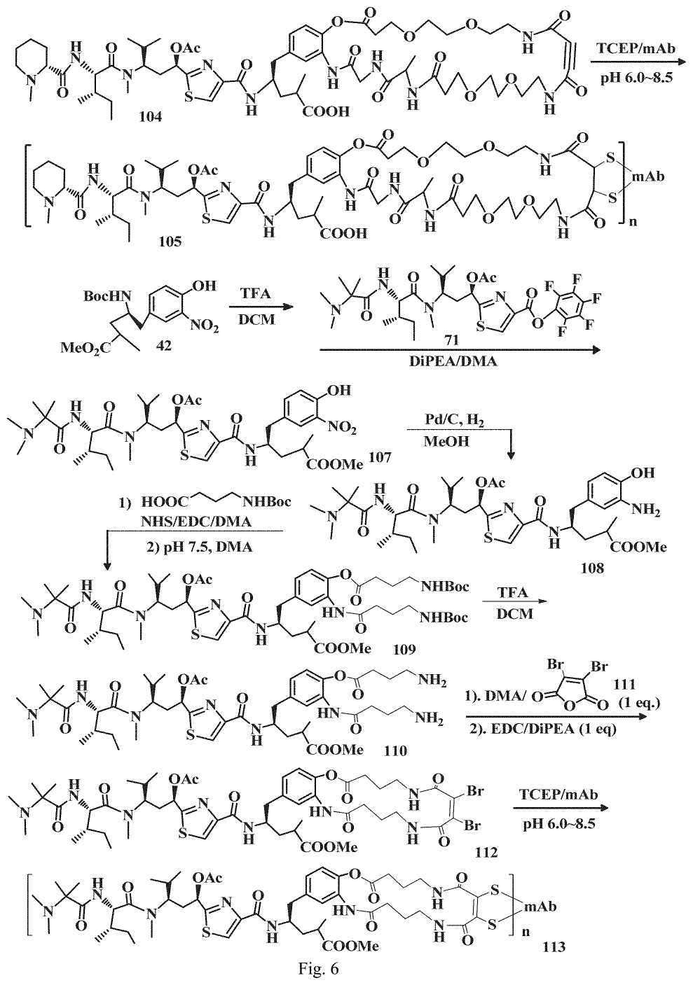

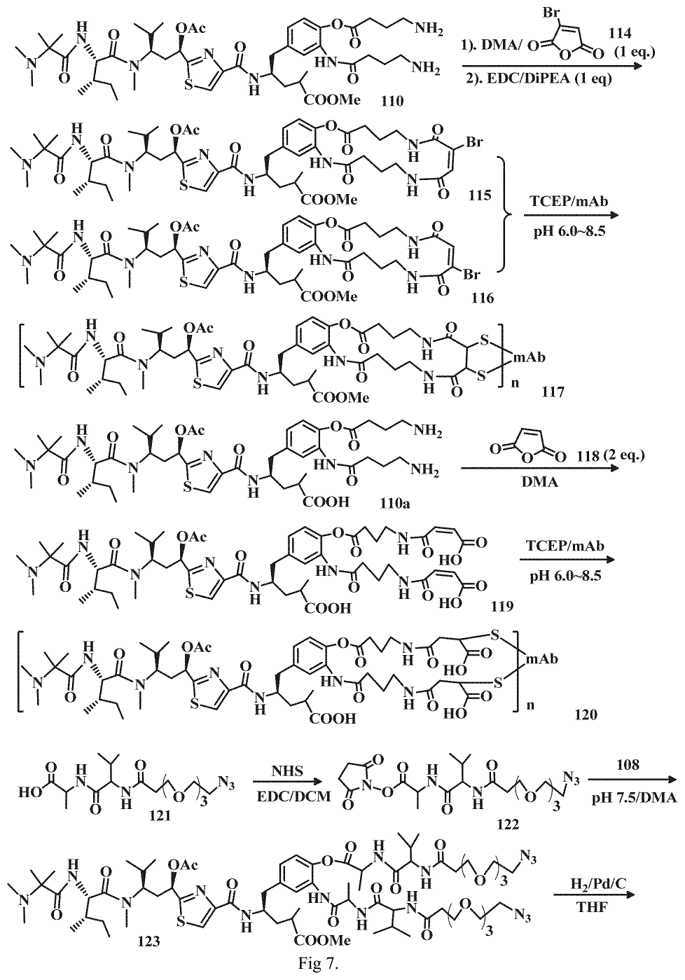

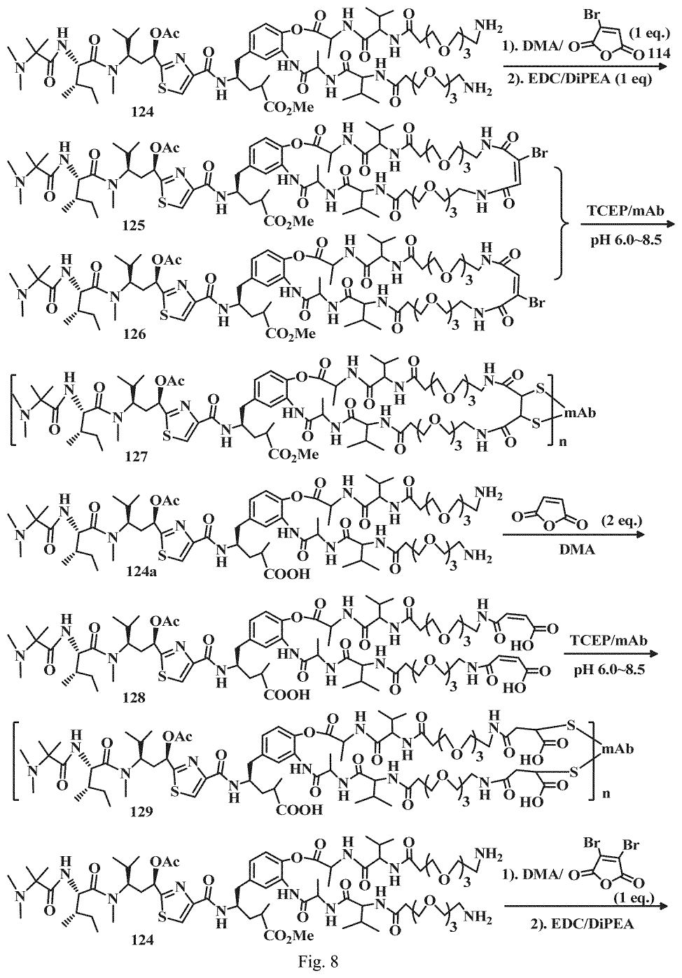

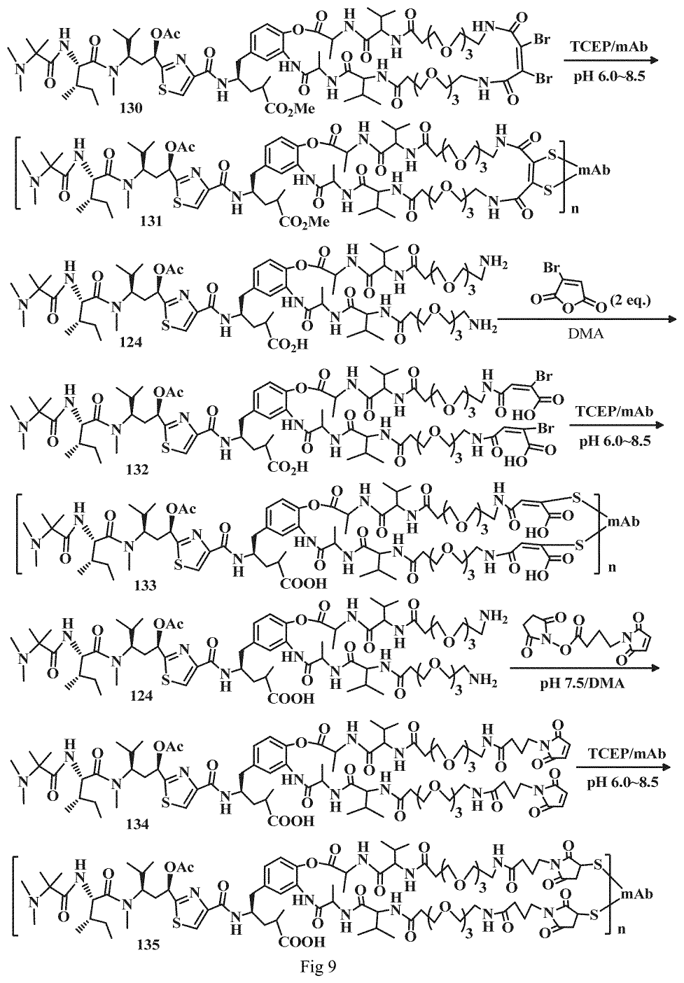

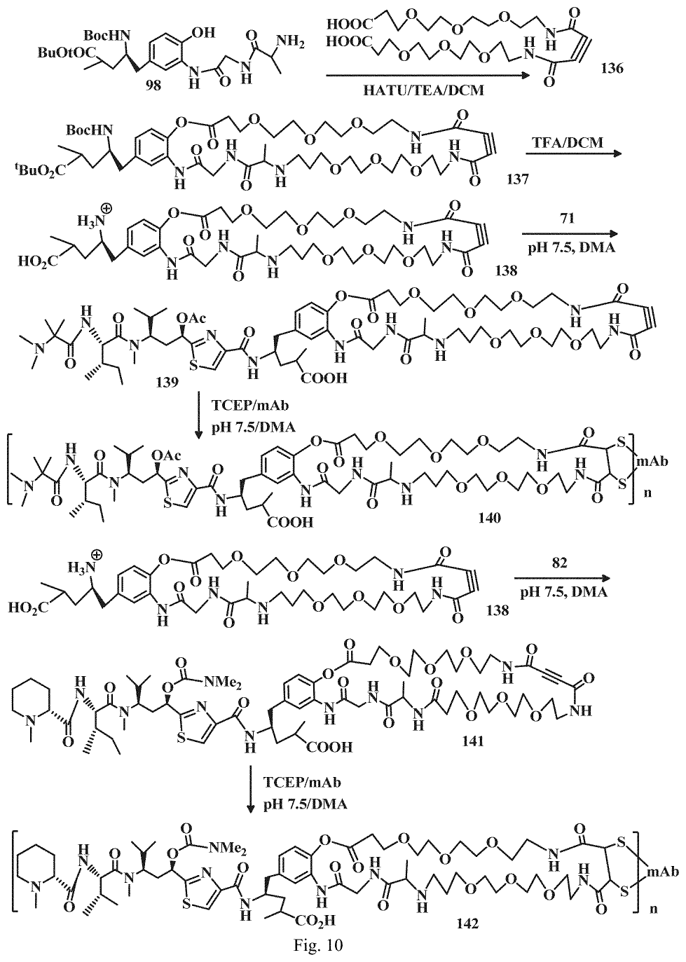

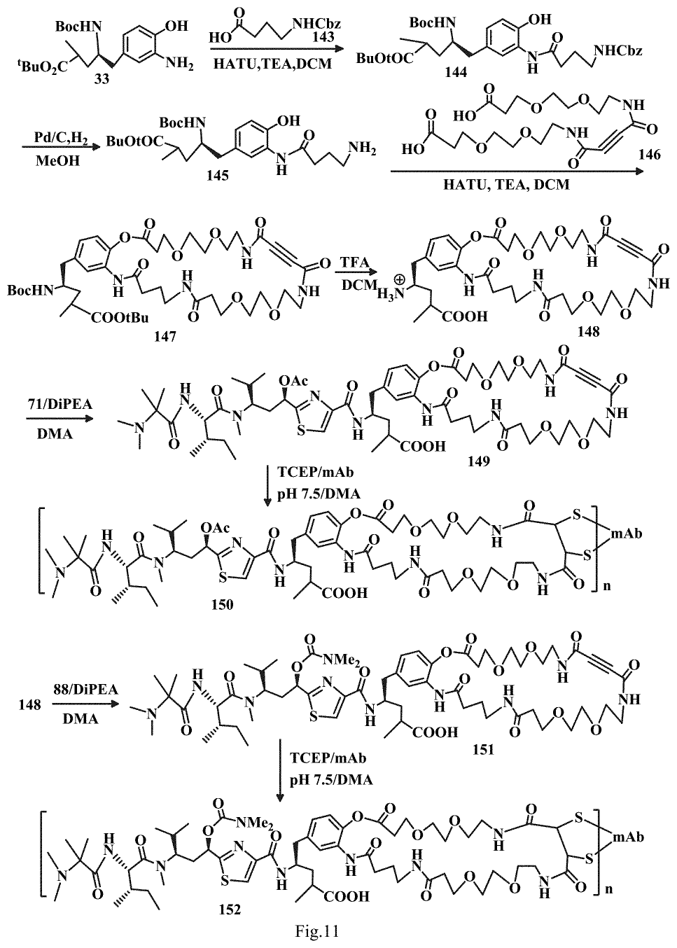

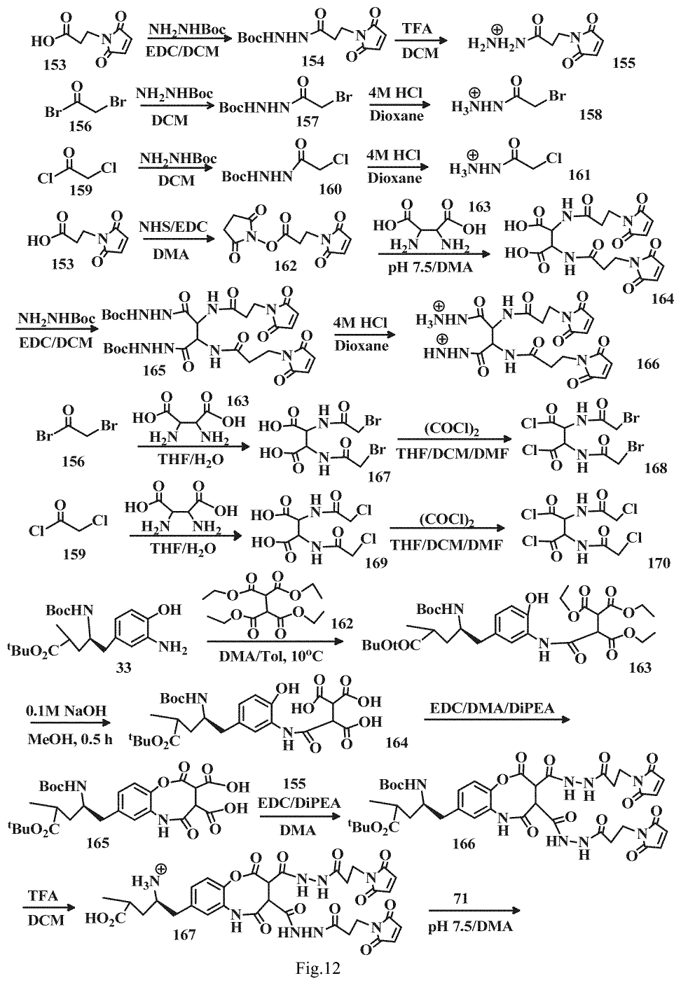

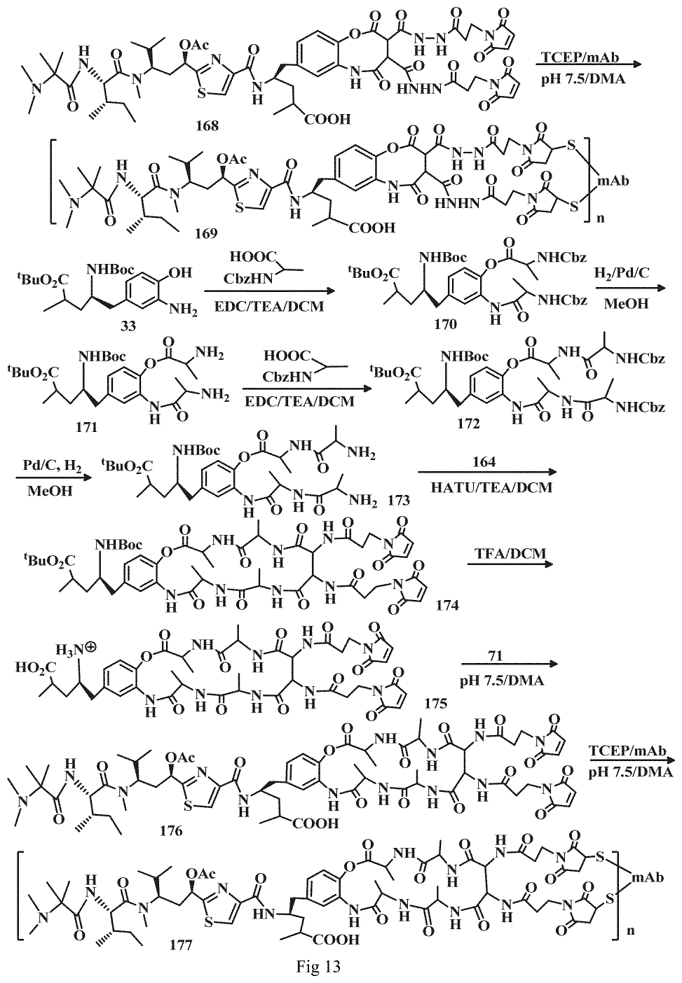

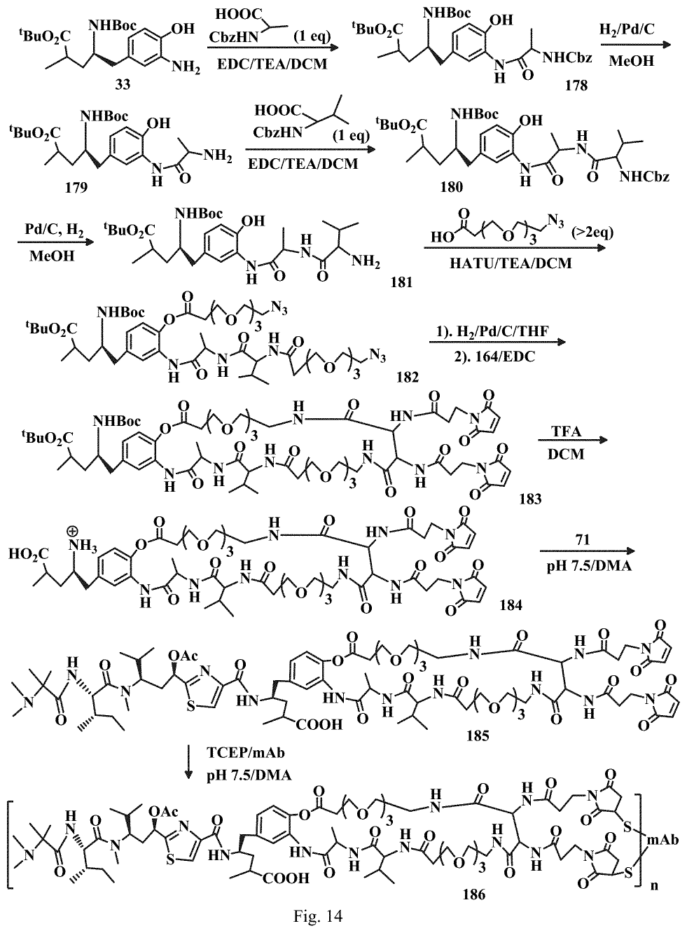

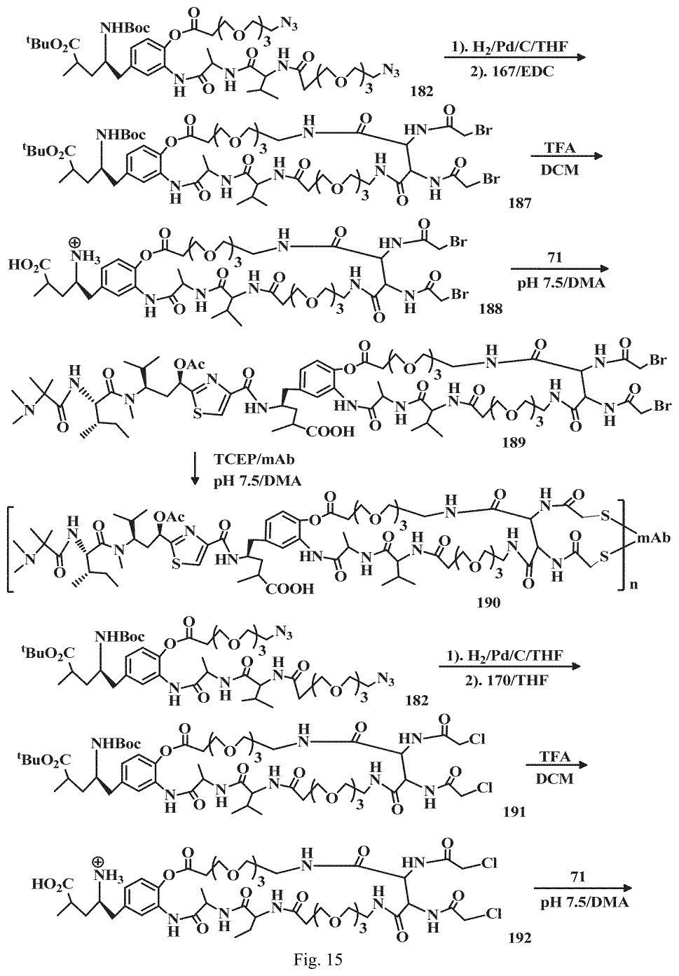

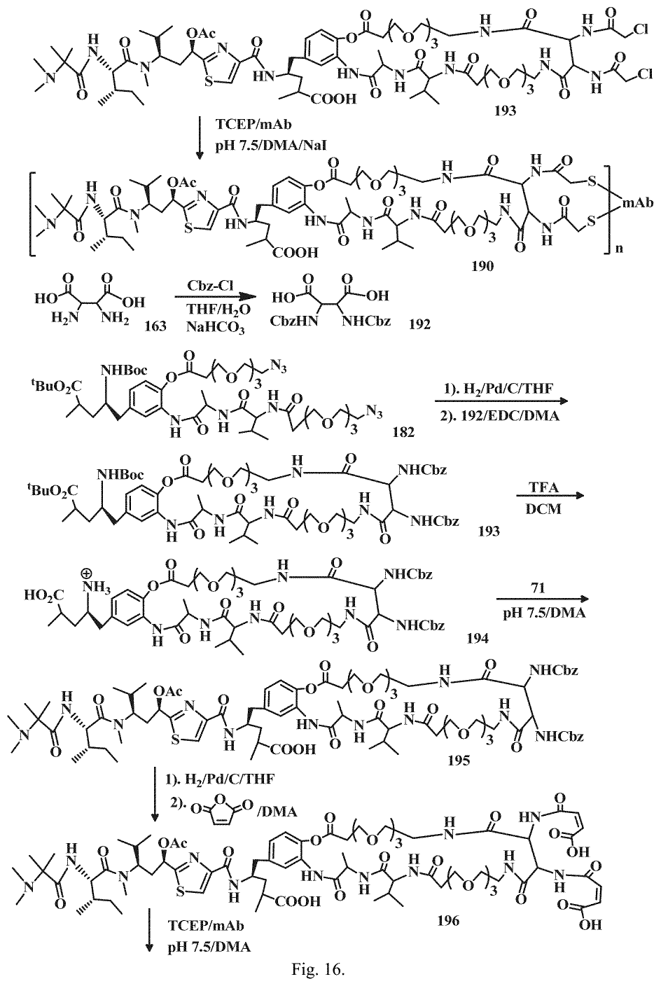

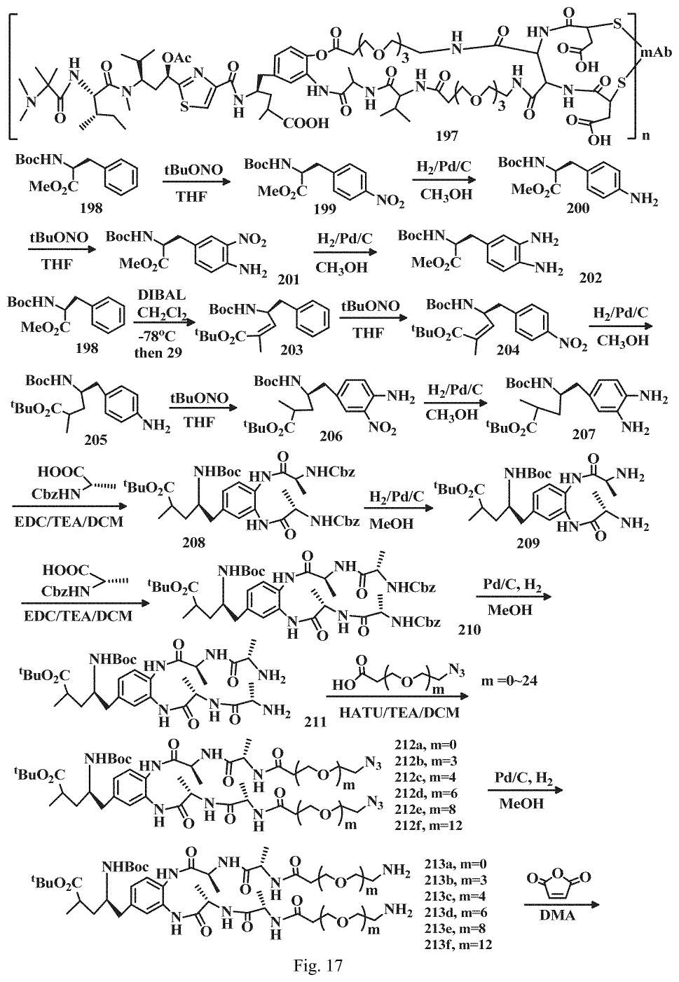

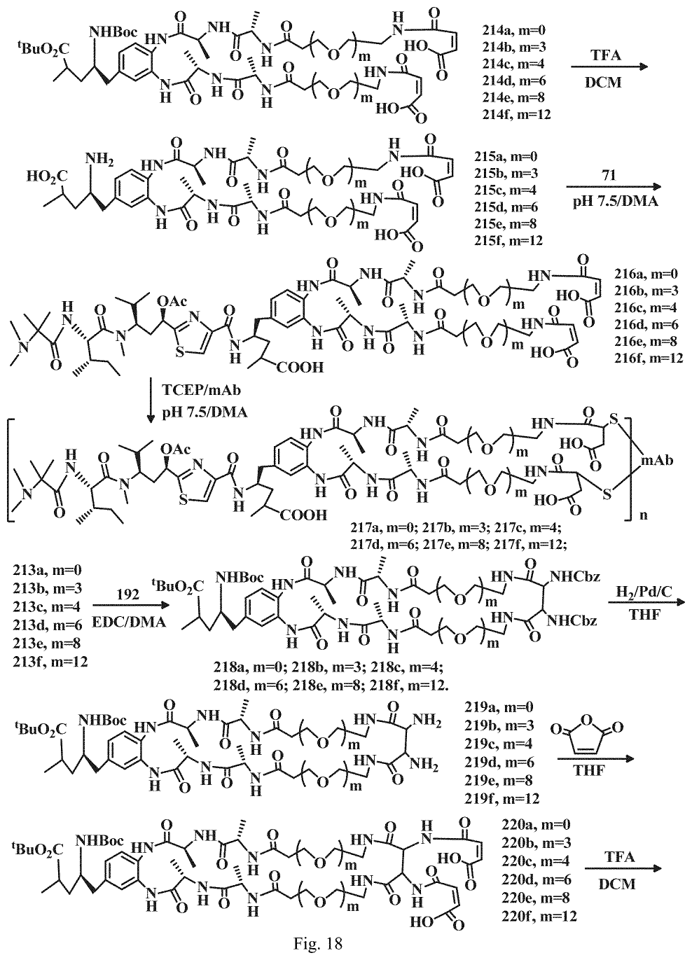

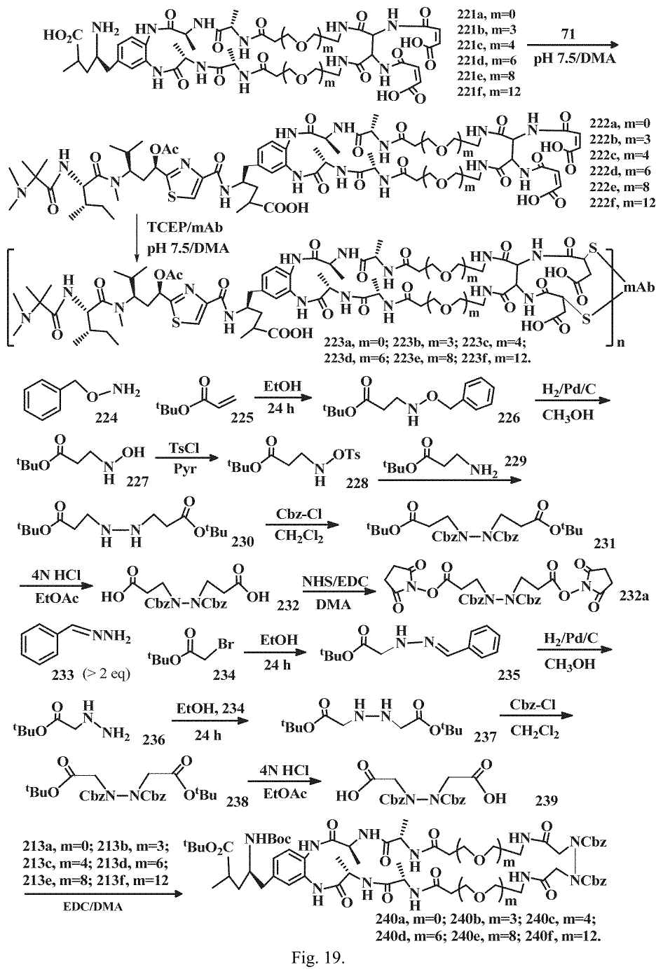

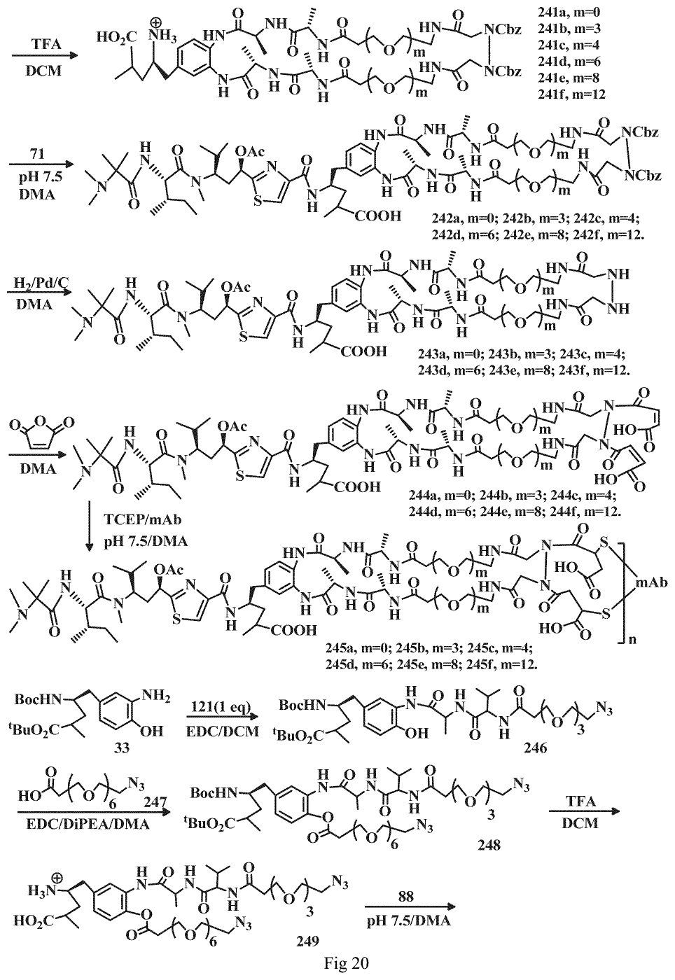

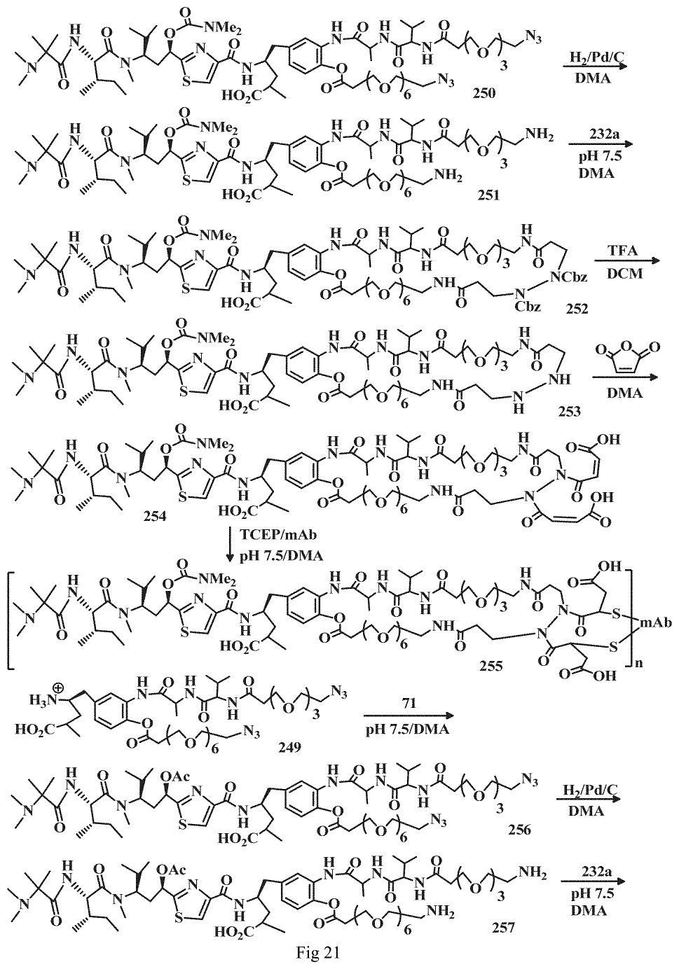

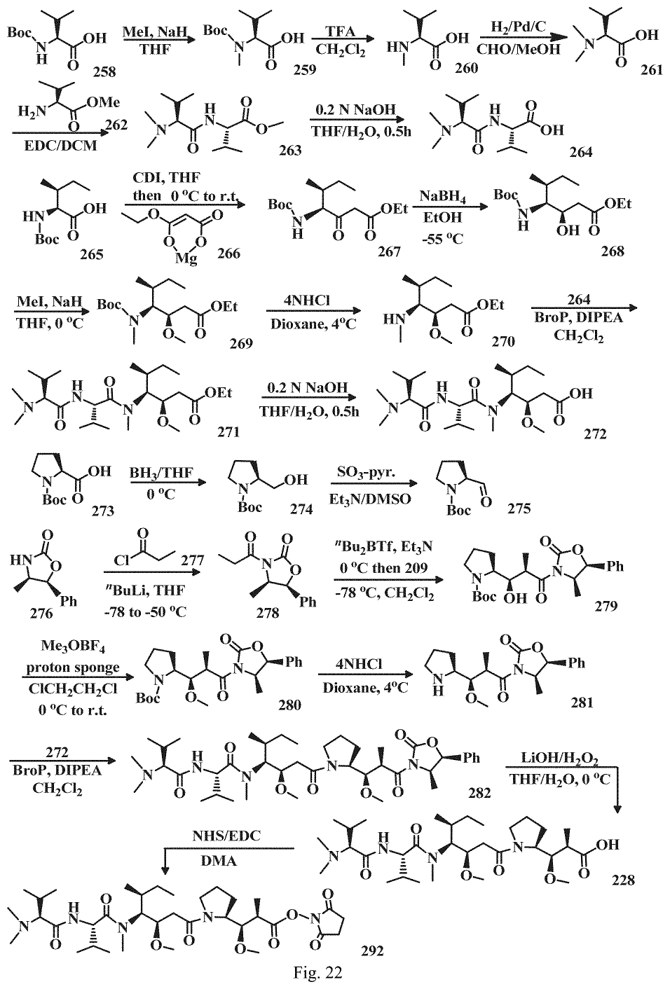

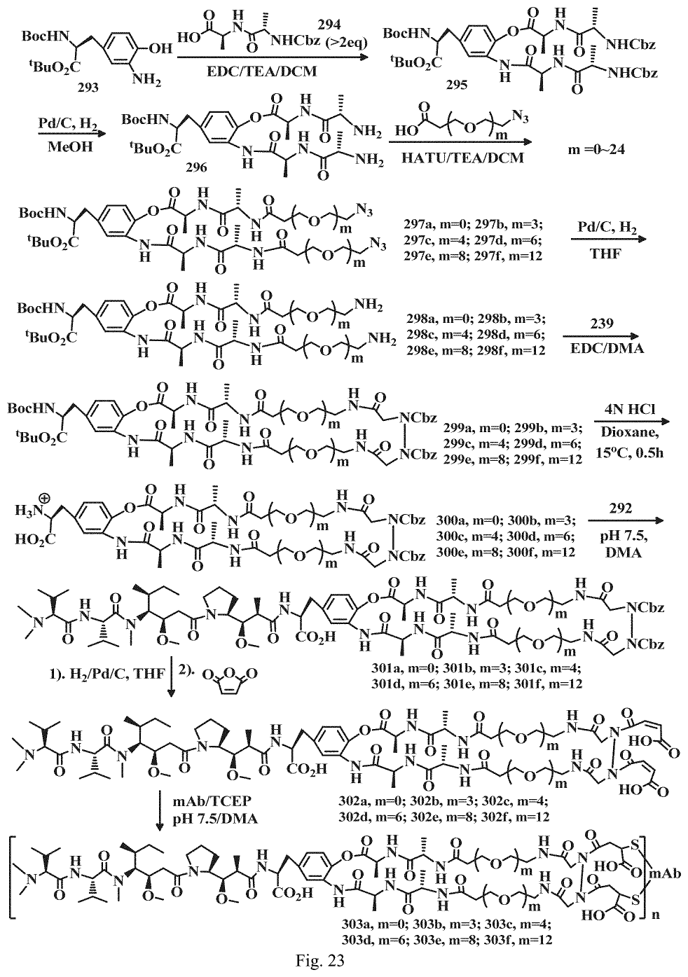

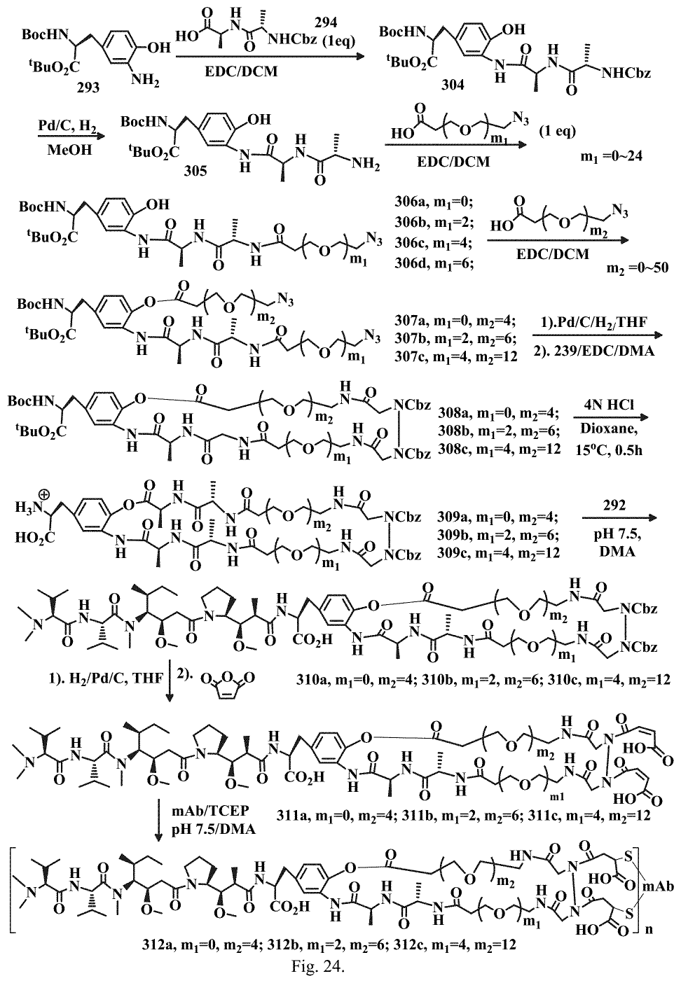

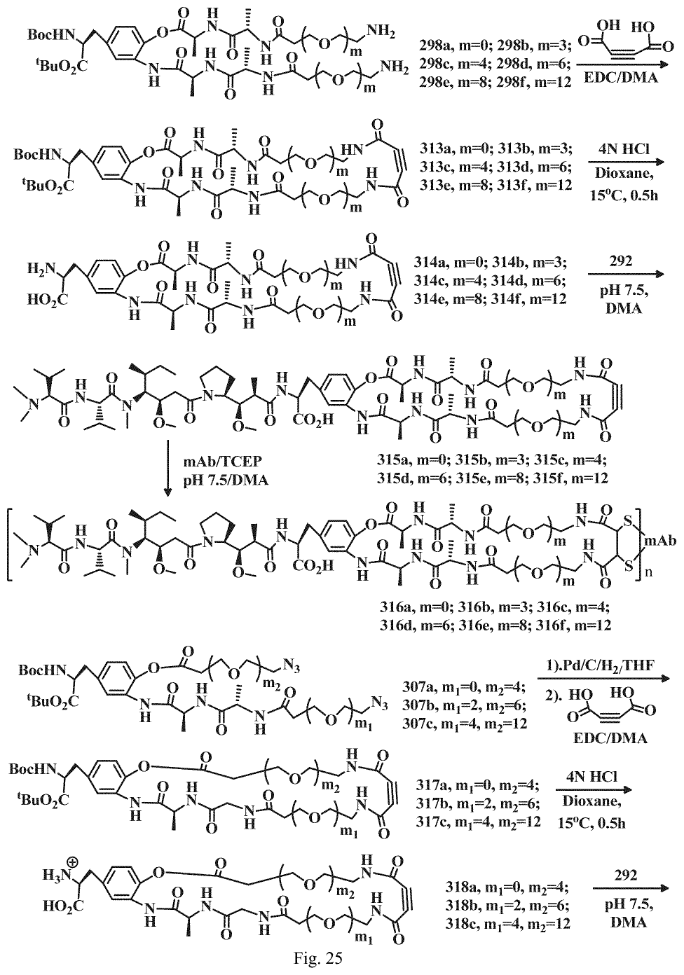

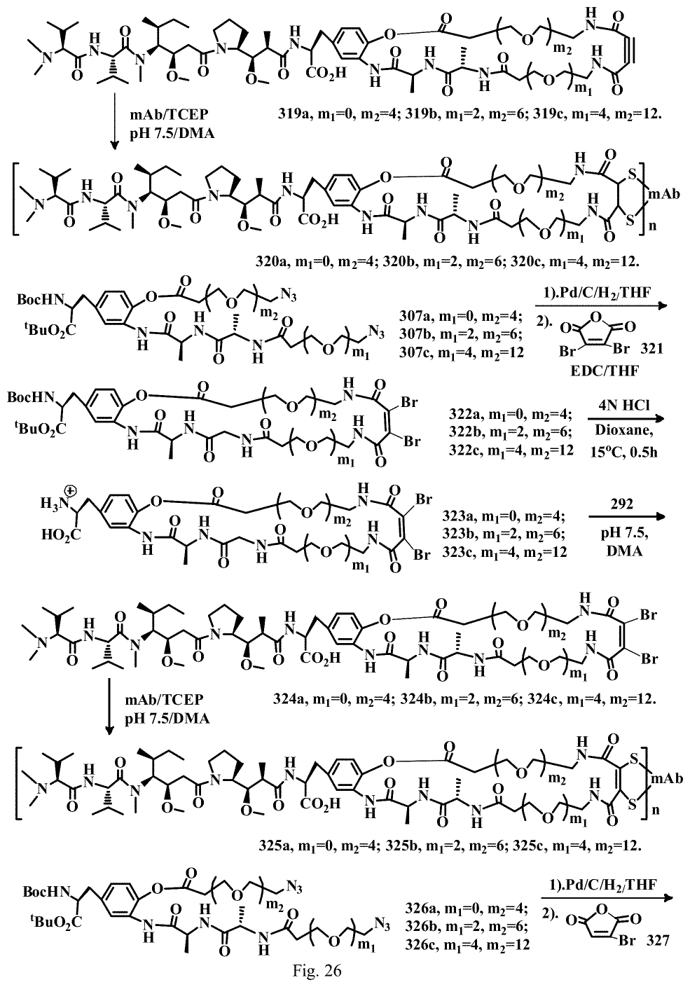

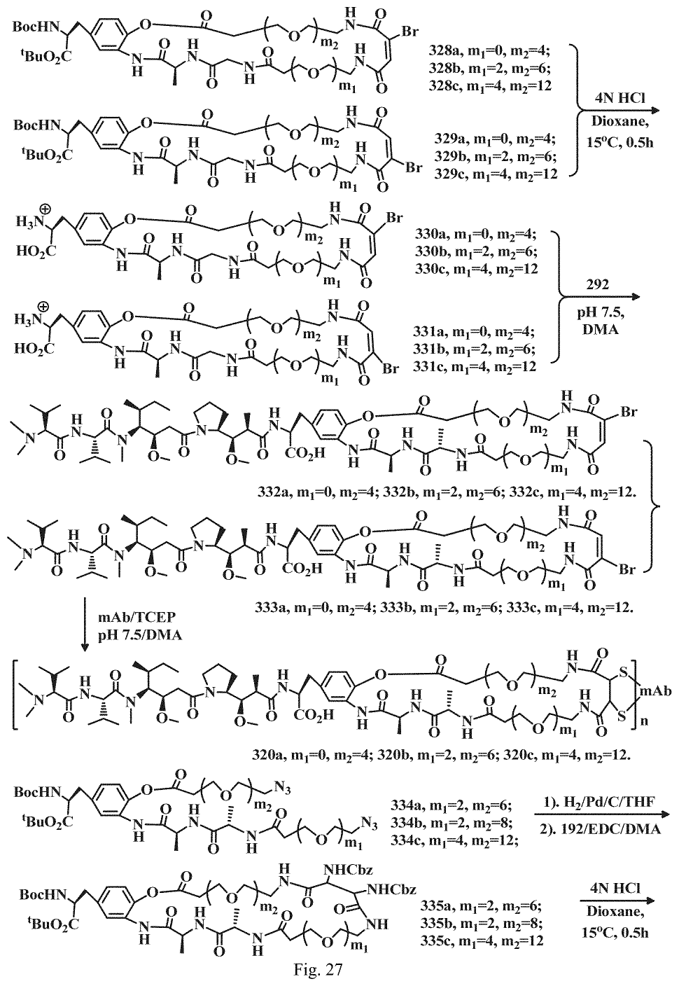

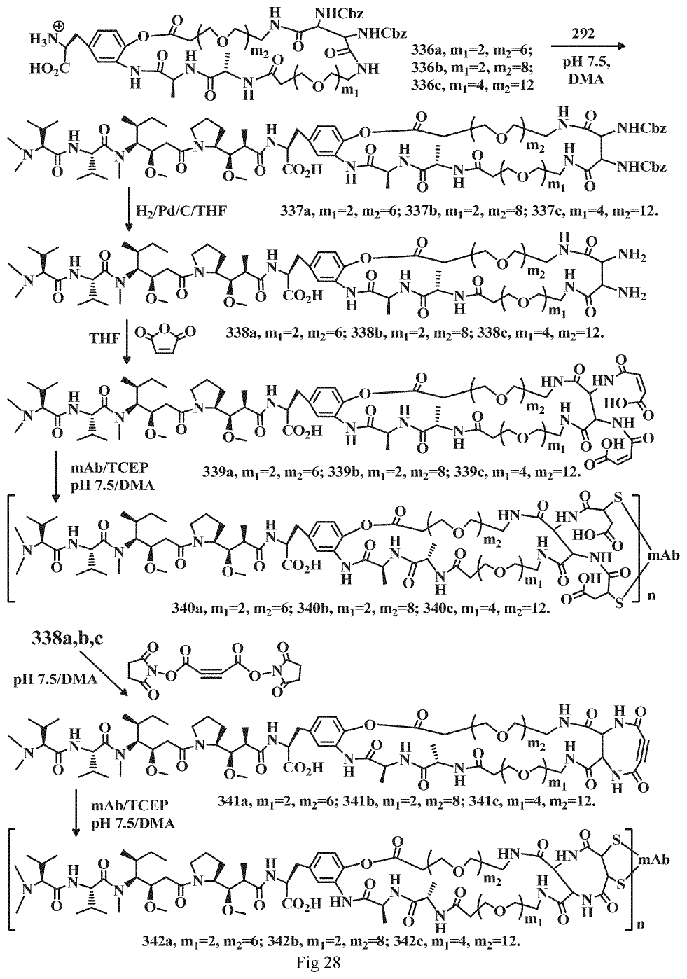

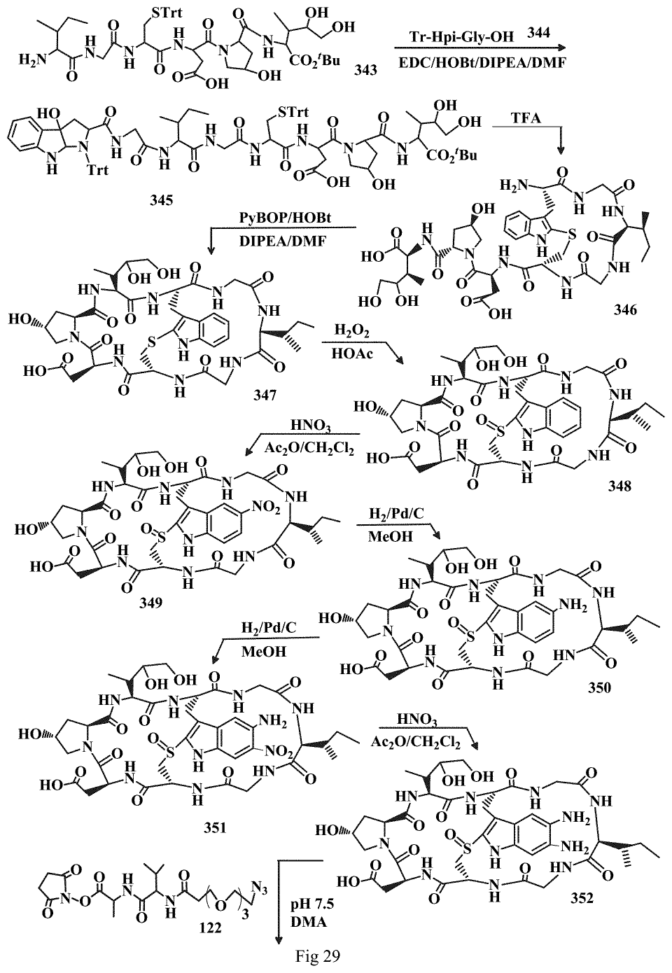

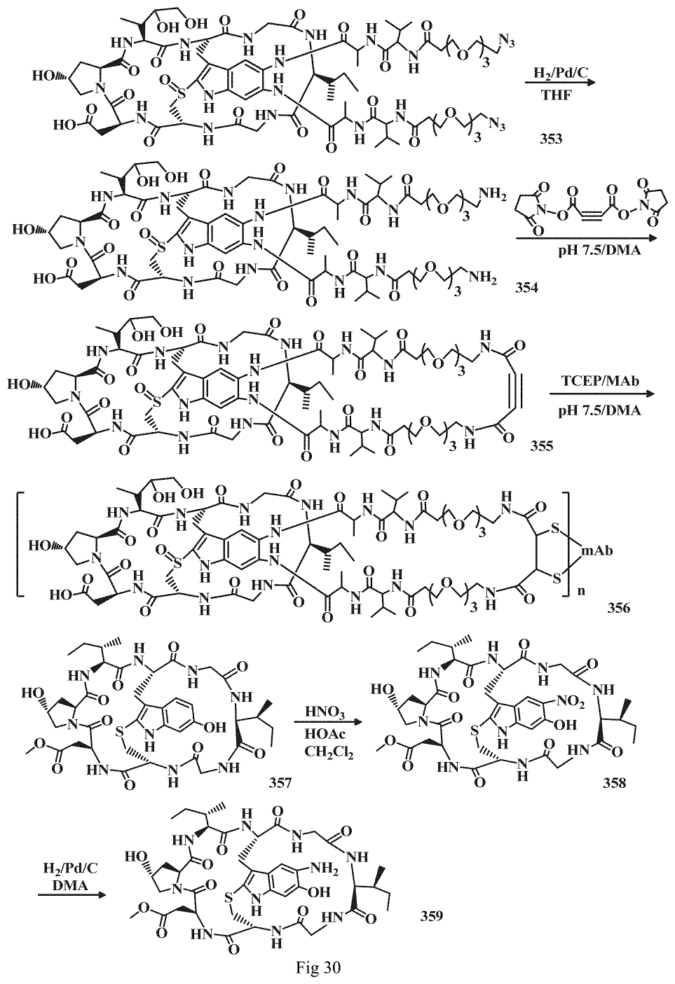

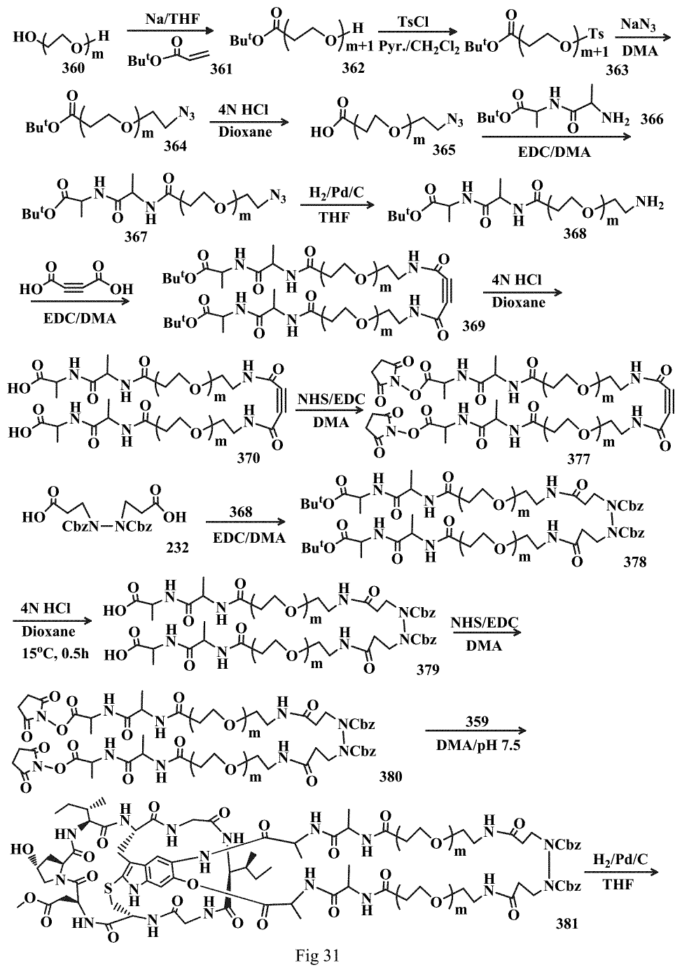

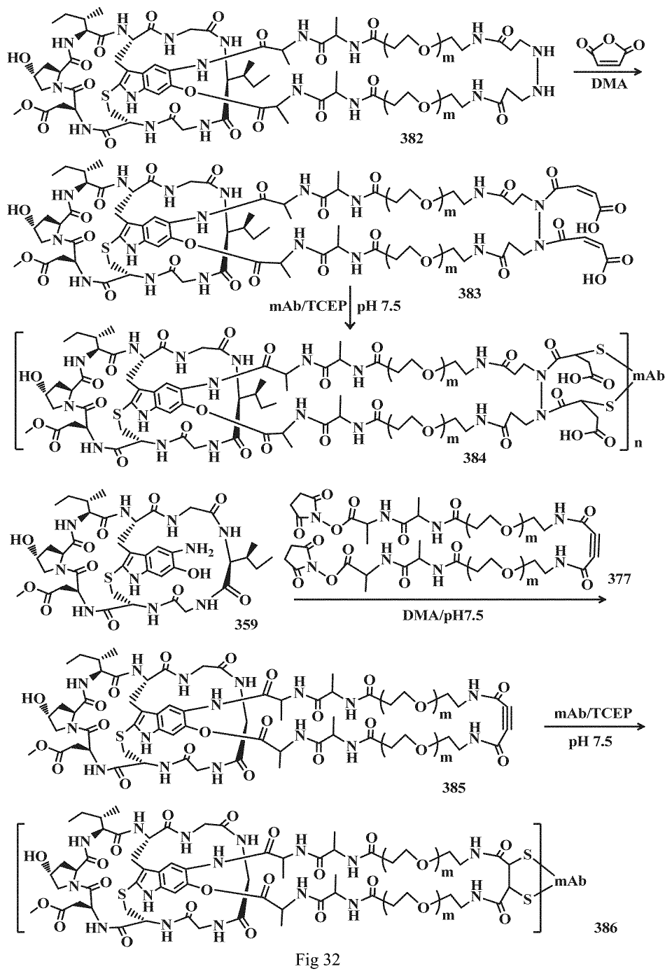

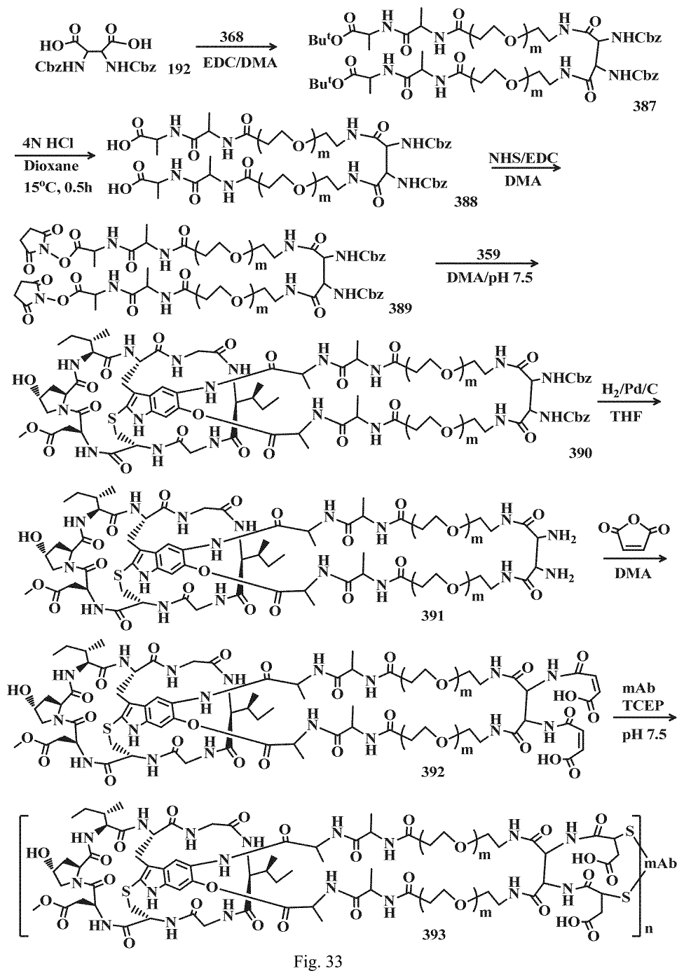

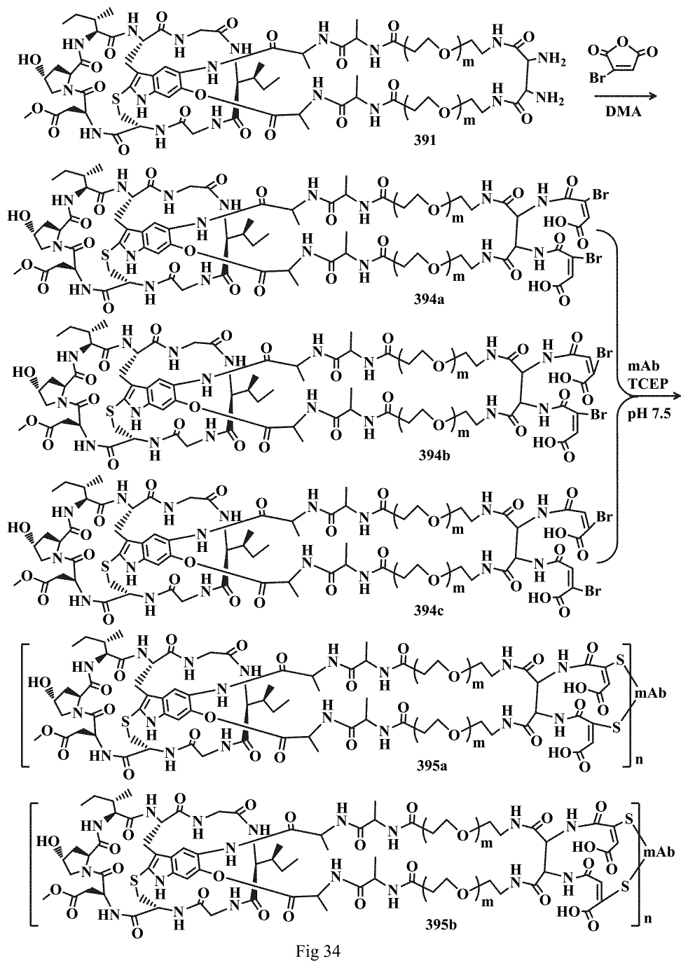

The novel conjugates disclosed herein use the bridge linkers. Examples of some suitable linkers and their synthesis are shown in FIGS. 1 to 34.

A Conjugate of a Cell-Binding Agent-A Cytotoxic Molecule Via the Bis-Linkage

The bis-linkage of the conjugate is represented by Formula (I):

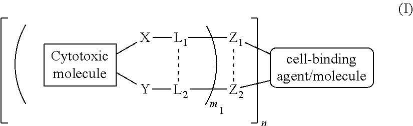

wherein

“ [symbol] ” represents a single bond;

“ [symbol] ” is optionally either a single bond, or a double bond, or can optionally be absent;

n and m₁ are 1 to 20 independently;

A cell-binding agent/molecule in the frame that links to Z₁ and Z₂ can be any kind presently known, or that become known, of a molecule that binds to, complexes with, or reacts with a moiety of a cell population sought to be therapeutically or otherwise biologically modified. Preferably the cell-binding agent/molecule is an immunotherapeutic protein, an antibody, a single chain antibody; an antibody fragment that binds to the target cell; a monoclonal antibody; a single chain monoclonal antibody; or a monoclonal antibody fragment that binds the target cell; a chimeric antibody; a chimeric antibody fragment that binds to the target cell; a domain antibody; a domain antibody fragment that binds to the target cell; adnectins that mimic antibodies; DARPins; a lymphokine; a hormone; a vitamin; a growth factor; a colony stimulating factor; or a nutrient-transport molecule (a transferrin); a binding peptides having over four aminoacids, or protein, or antibody, or small cell-binding molecule or ligand attached on albumin, polymers, dendrimers, liposomes, nanoparticles, vesicles, or (viral) capsids;

A cytotoxic molecule/agent in the frame is a therapeutic drug/molecule/agent, or an immunotherapeutic protein/molecule, or a function molecule for enhancement of binding or stabilization of the cell-binding agent, or a cell-surface receptor binding ligand, or for inhibition of cell proliferation, or for monitoring, detection or study of a cell-binding molecule action. It can also be an analog, or prodrug, or a pharmaceutically acceptable salt, hydrate, or hydrated salt, or a crystalline structure, or an optical isomer, racemate, diastereomer or enantiomer, of immunotherapeutic compound, a chemotherapeutic compound, an antibody (probody) or an antibody (probody) fragment, or siRNA or DNA molecule, or a cell surface binding ligand;

Preferably a cytotoxic molecule is any of many small molecule drugs, including, but not limited to, tubulysins, calicheamicins, auristatins, maytansinoids, CC-1065 analogs, morpholinos doxorubicins, taxanes, cryptophycins, amatoxins (e.g. amanitins), epothilones, eribulin, geldanamycins, duocarmycins, daunomycins, methotrexates, vindesines, vincristines, and benzodiazepine dimers (e.g., dimers of pyrrolobenzodiazepine (PBD), tomaymycin, indolinobenzodiazepines, imidazobenzothiadiazepines, or oxazolidinobenzodiazepines);

X and Y, represent the same or different, and independently, a functional group that links a cytotoxic drug via a disulfide, thioether, thioester, peptide, hydrazone, ether, ester, carbamate, carbonate, amine (secondary, tertiary, or quartary), imine, cycloheteroalkyane, heteroaromatic, alkoxime or amide bond; Preferably X and Y are independently selected from NH; NHNH; N(R₁); N(R₁)N(R₂); O; S; S—S, O—NH. O—N(R₁), CH₂—NH. CH₂—N(R₁), CH═NH. CH═N(R₁), S(O), S(O₂), P(O)(OH), S(O)NH, S(O₂)NH, P(O)(OH)NH, NHS(O)NH, NHS(O₂)NH, NHP(O)(OH)NH, N(R₁)S(O)N(R₂), N(R₁)S(O₂)N(R₂), N(R₁)P(O)(OH)N(R₂), OS(O)NH, OS(O₂)NH, OP(O)(OH)NH, C(O), C(NH), C(NR₁), C(O)NH, C(NH)NH, C(NR₁)NH, OC(O)NH, OC(NH)NH; OC(NR₁)NH, NHC(O)NH; NHC(NH)NH; NHC(NR₁)NH, C(O)NH, C(NH)NH, C(NR₁)NH, OC(O)N(R₁), OC(NH)N(R₁), OC(NR₁)N(R₁), NHC(O)N(R₁), NHC(NH)N(R₁), NHC(NR₁)N(R₁), N(R₁)C(O)N(R₁), N(R₁)C(NH)N(R₁), N(R₁)C(NR₁)N(R₁); or C₁-C₆ alkyl, C₂-C₈ alkenyl, heteroalkyl, alkylcycloalkyl, or heterocycloalkyl; C₃-C₈ aryl, Ar-alkyl, heterocyclic, carbocyclic, cycloalkyl, heteroalkylcycloalkyl, alkylcarbonyl, or heteroaryl;

Z₁ and Z₂ are, the same or different, and independently a function group that have linked to a cell-binding molecule, to form a disulfide, ether, ester, thioether, thioester, peptide, hydrazone, carbamate, carbonate, amine (secondary, tertiary, or quarter), imine, cycloheteroalkyane, heteroaromatic, alkyloxime or amide bond; Preferably Z₁ and Z₂ independently have the following structures: C(O)CH, C(O)C, C(O)CH₂, ArCH₂, C(O), NH; NHNH; N(R₁); N(R₁)N(R₂); O; S; S—S, O—NH. O—N(R₁), CH₂—NH. CH₂—N(R₁), CH═NH. CH═N(R₁), S(O), S(O₂), P(O)(OH), S(O)NH, S(O₂)NH, P(O)(OH)NH, NHS(O)NH, NHS(O₂)NH, NHP(O)(OH)NH, N(R₁)S(O)N(R₂), N(R₁)S(O₂)N(R₂), N(R₁)P(O)(OH)N(R₂), OS(O)NH, OS(O₂)NH, OP(O)(OH)NH, C(O), C(NH), C(NR₁), C(O)NH, C(NH)NH, C(NR₁)NH, OC(O)NH, OC(NH)NH; OC(NR₁)NH, NHC(O)NH; NHC(NH)NH; NHC(NR₁)NH, C(O)NH, C(NH)NH, C(NR₁)NH, OC(O)N(R₁), OC(NH)N(R₁), OC(NR₁)N(R₁), NHC(O)N(R₁), NHC(NH)N(R₁), NHC(NR₁)N(R₁), N(R₁)C(O)N(R₁), N(R₁)C(NH)N(R₁), N(R₁)C(NR₁)N(R₁); or C₁-C₈ alkyl, C₂-C₈ heteroalkyl, alkylcycloalkyl, or heterocycloalkyl; C₃-C₈ aryl, Ar-alkyl, heterocyclic, carbocyclic, cycloalkyl, heteroalkylcycloalkyl, alkylcarbonyl, or heteroaryl;

Preferably Z₁ and Z₂ are linked to pairs of thiols of a cell-binding agent/molecule. The thiols are preferably pairs of sulfur atoms reduced from the inter chain disulfide bonds of the cell-binding agent by a reduction agent selected from dithiothreitol (DTT), dithioerythritol (DTE), L-glutathione (GSH), tris (2-carboxyethyl) phosphine (TCEP), 2-mercaptoethylamine (β-MEA), or/and beta mercaptoethanol (β-ME, 2-ME);

L₁ and L₂ are a chain of atoms selected from C, N, O, S, Si, and P, having 0˜500 atoms, which covalently connects to X and Z₁, and Y and Z₂. The atoms used in forming the L₁ and L₂ may be combined in all chemically relevant ways, preferably are C₁-C₂₀ alkylene, alkenylene, and alkynylene, ethers, polyoxyalkylene, esters, amines, imines, polyamines, hydrazines, hydrazones, amides, ureas, semicarbazides, carbazides, alkoxyamines, alkoxylamines, urethanes, amino acids, peptides, acyloxylamines, hydroxamic acids, or combination above thereof. More preferably L₁ and L₂ are, the same or different, independently selected from O, NH, S, NHNH, N(R₃), N(R₃)N(R_{3′}), C₁-C₈ alkyl, amide, amines, imines, hydrazines, hydrazones; C₂-C₈ heteroalkyl, alkylcycloalkyl, ethers, esters, hydrazones, ureas, semicarbazides, carbazides, alkoxyamines, alkoxylamines, urethanes, amino acids, peptides, acyloxylamines, hydroxamic acids, or heterocycloalkyl; C₃-C₈ aryl, Ar-alkyl, heterocyclic, carbocyclic, cycloalkyl, heteroalkylcycloalkyl, alkylcarbonyl, or heteroaryl; polyethyleneoxy unit of formula (OCH₂CH₂)ₚOR₃, or (OCH₂CH(CH₃))ₚOR₃, or NH(CH₂CH₂O)ₚR₃, or NH(CH₂CH(CH₃)O)ₚR₃, or N[(CH₂CH₂O)ₚR₃]—[(CH₂CH₂O)_{p′}R_{3′}], or (OCH₂CH₂)ₚCOOR₃, or CH₂CH₂(OCH₂CH₂)ₚCOOR₃, wherein p and p′ are independently an integer selected from 0 to about 5000, or combination thereof; wherein R₃ and R_{3′} are independently H; C₁-C₈ alkyl; C₂-C₈ heteroalkyl, alkylcycloalkyl, or heterocycloalkyl; C₃-C₈ aryl, Ar-alkyl, heterocyclic, carbocyclic, cycloalkyl, heteroalkylcycloalkyl, alkylcarbonyl, or heteroaryl; or C₂-C₈ esters, ether, or amide; or 1˜8 amino acids; or polyethyleneoxy having formula (OCH₂CH₂)ₚ or (OCH₂CH(CH₃))ₚ, wherein p is an integer from 0 to about 5000, or combination above thereof;

Optionally L₁ and L₂ may independently be composed of one or more linker components of 6-maleimidocaproyl (“MC”), maleimidopropanoyl (“MP”), valine-citrulline (“val-cit” or “vc”), alanine-phenylalanine (“ala-phe” or “af”), p-aminobenzyloxycarbonyl (“PAB”), 4-thiopentanoate (“SPP”), 4-(N-maleimidomethyl)cyclohexane-1 carboxylate (“MCC”), (4-acetyl)amino-benzoate (“SIAB”), 4-thio-butyrate (SPDB), 4-thio-2-hydroxysulfonyl-butyrate (2-Sulfo-SPDB), or natural or unnatural peptides having 1˜8 natural or unnatural amino acid unites. The natural aminoacid is preferably selected from aspartic acid, glutamic acid, arginine, histidine, lysine, serine, threonine, asparagine, glutamine, cysteine, selenocysteine, tyrosine, phenylalanine, glycine, proline, tryptophan, alanine;

L₁ and L₂ may also independently contain a self-immolative or a non-self-immolative component, peptidic units, a hydrazone bond, a disulfide, an ester, an oxime, an amide, or a thioether bond. The self-immolative unit includes, but is not limited to, aromatic compounds that are electronically similar to the para-aminobenzylcarbamoyl (PAB) groups such as 2-aminoimidazol-5-methanol derivatives, heterocyclic PAB analogs, beta-glucuronide, and ortho or para-aminobenzylacetals;

Preferably, the self-immolative linker component has one of the following structures:

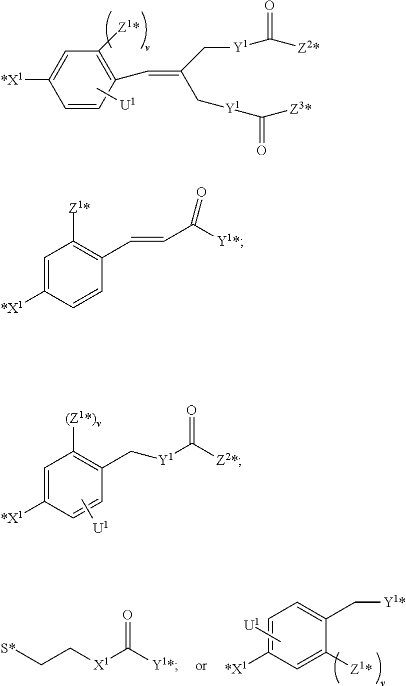

wherein the (*) atom is the point of attachment of additional spacer or releasable linker units, or the cytotoxic agent, and/or the binding molecule (CBA); X¹, Y¹, Z² and Z³ are independently NH, O, or S; Z¹ is independently H, NHR₁, OR₁, SR₁, COX₁R₁, wherein X₁ and R₁ are defined above; v is 0 or 1; U¹ is independently H, OH, C₁˜C₆ alkyl, (OCH₂CH₂)ₙ, F, Cl, Br, I, OR₅, SR₅, NR₅R₅′, N═NR₅, N═R₅, NR₅R₅′, NO₂, SOR₅R₅′, SO₂R₅, SO₃R₅, OSO₃R₅, PR₅R₅′, POR₅R₅′, PO₂R₅R₅′, OPO(OR₅)(OR₅′), or OCH₂PO(OR₅(OR₅′), wherein R₅ and R₅′ are independently selected from H, C₁˜C₈ alkyl; C₂˜C₈ alkenyl, alkynyl, heteroalkyl, or amino acid; C₃˜C₈ aryl, heterocyclic, carbocyclic, cycloalkyl, heterocycloalkyl, heteroaralkyl, alkylcarbonyl, or glycoside; or pharmaceutical cation salts;

The non-self-immolative linker component is one of the following structures:

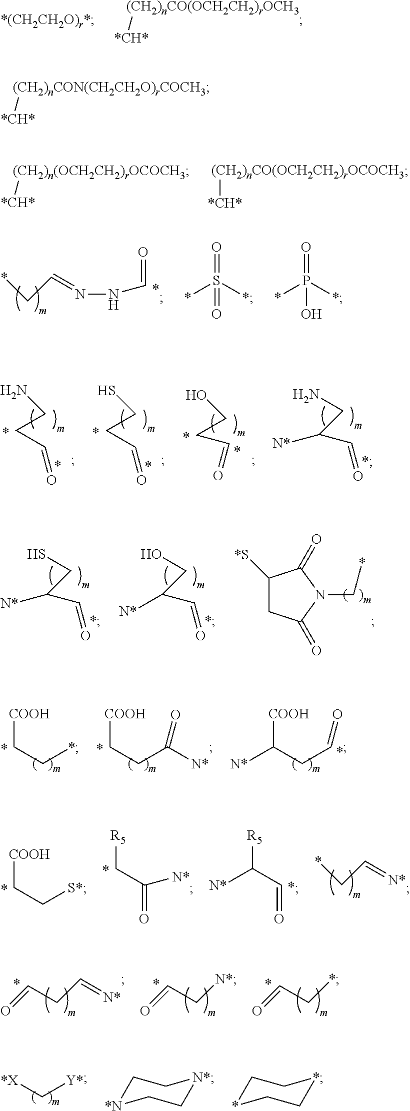

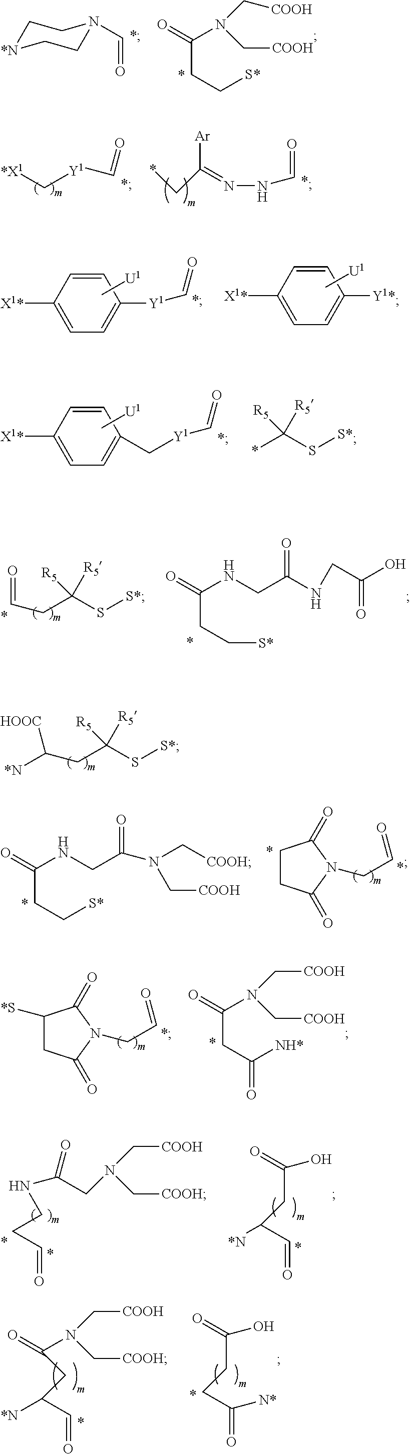

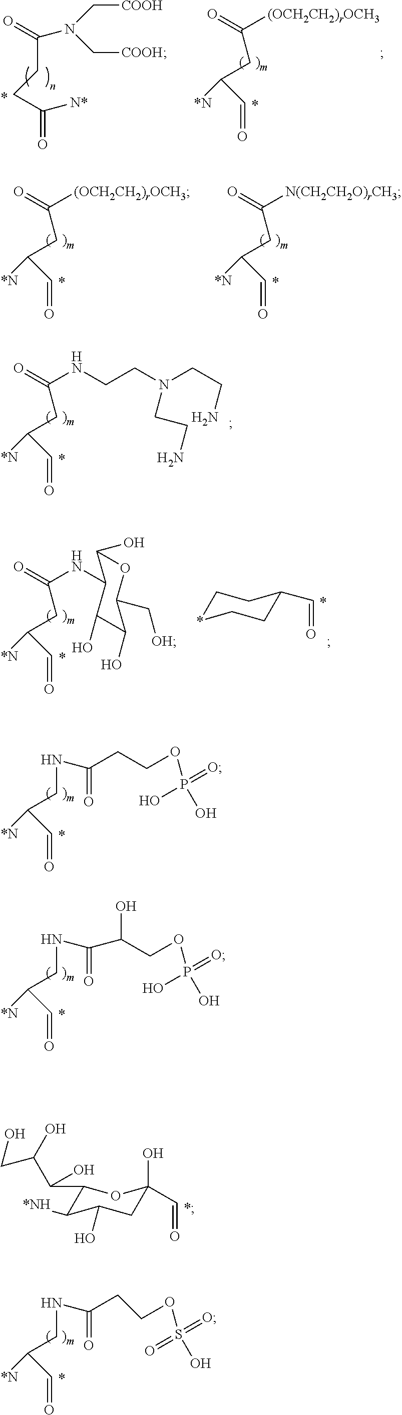

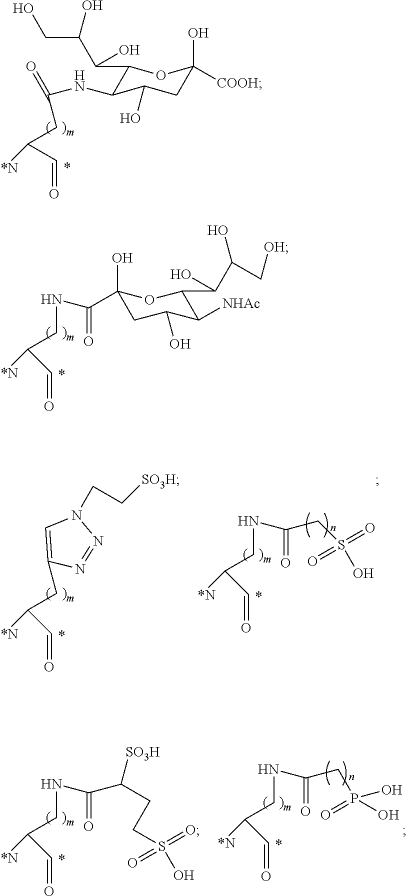

wherein the (*) atom is the point of attachment of additional spacer or releasable linkers, the cytotoxic agents, and/or the binding molecules; X¹, Y¹, U¹, R₅, R₅′ are defined as above; r is 0˜100; m and n are 0˜6 independently;

Further preferably, L₁ and L₂ may independently be a releasable linker. The term releasable linker refers to a linker that includes at least one bond that can be broken under physiological conditions, such as a pH-labile, acid-labile, base-labile, oxidatively labile, metabolically labile, biochemically labile or enzyme-labile bond. It is appreciated that such physiological conditions resulting in bond breaking do not necessarily include a biological or metabolic process, and instead may include a standard chemical reaction, such as a hydrolysis or substitution reaction, for example, an endosome having a lower pH than cytosolic pH, and/or disulfide bond exchange reaction with a intracellular thiol, such as a millimolar range of abundant of glutathione inside the malignant cells;

Examples of the releasable linkers L₁ or L₂ include, but not limited:

—(CR₅R₆)ₘ(Aa)r(CR₇R₈)ₙ(OCH₂CH₂)ₜ—, —(CR₅R₆)ₘ(CR₇R₈)ₙ(Aa)ᵣ(OCH₂CH₂)ₜ—, -(Aa)ᵣ-(CR₅R₆)ₘ(CR₇R₈)ₙ(OCH₂CH₂)ₜ—, —(CR₅R₆)ₘ(CR₇R₈)ₙ(OCH₂CH₂)ᵣ(Aa)ₜ-, —(CR₅R₆)ₘ—(CR₇═CR₈)(CR₉R₁₀)ₙ(Aa)ₜ(OCH₂CH₂)ᵣ—, —(CR₅R₆)ₘ(NR₁₁CO)(Aa)ₜ(CR₉R₁₀)ₙ—(OCH₂CH₂)ᵣ—, —(CR₅R₆)ₘ(Aa)ₜ(NR₁₁CO)(CR₉R₁₀)ₙ(OCH₂CH₂)ᵣ—, —(CR₅R₆)ₘ(OCO)(Aa)ₜ(CR₉R₁₀)ₙ—(OCH₂CH₂)ᵣ—, —(CR₅R₆)ₘ(OCNR₇)(Aa)ₜ(CR₉R₁₀)ₙ(OCH₂CH₂)ᵣ—, —(CR₅R₆)ₘ(CO)(Aa)ₜ-(CR₉R₁₀)ₙ(OCH₂CH₂)ᵣ—, —(CR₅R₆)ₘ(NR₁₁CO)(Aa)ₜ(CR₉R₁₀)ₙ(OCH₂CH₂)ᵣ—, —(CR₅R₆)ₘ—(OCO)(Aa)ₜ(CR₉R₁₀)ₙ—(OCH₂CH₂)ᵣ—, —(CR₅R₆)ₘ(OCNR₇)(Aa)ₜ(CR₉R₁₀)ₙ(OCH₂CH₂)ᵣ—, —(CR₅R₆)ₘ(CO)(Aa)ₜ(CR₉R₁₀)ₙ—(OCH₂CH₂)ᵣ—, —(CR₅R₆)ₘphenyl-CO(Aa)ₜ(CR₇R₈)ₙ—, —(CR₅R₆)ₙ-furyl-CO(Aa)ₜ(CR₇R₈)ₙ—, —(CR₅R₆)ₘ-oxazolyl-CO(Aa)ₜ(CR₇R₈)ₙ—, —(CR₅R₆)ₘ-thiazolyl-CO(Aa)(CCR₇R₈)ₙ—, —(CR₅R₆)ₜ-thienyl-CO(CR₇R₈)ₙ—, —(CR₅R₆)ₜ-imidazolyl-CO—(CR₇R₈)ₙ—, —(CR₅R₆)ₜ-morpholino-CO(Aa)ₜ-. (CR₇R₈)ₙ—, —(CR₅R₆)ₜpiperazino-CO(Aa)ₜ(CR₇R₈)ₙ—, —(CR₅R₆)ₜ—N-methylpiperazin-CO(Aa)ₜ-(CR₇R₈)ₙ—, —(CR₅R₆)ₘ-(Aa)ₜphenyl-, —(CR₅R₆)ₘ-(Aa)ₜfuryl-, —(CR₅R₆)ₘ-oxazolyl(Aa)ₜ-, —(CR₅R₆)ₘ-thiazolyl(Aa)ₜ-, —(CR₅R₆)ₘ-thienyl-(Aa)ₜ-, —(CR₅R₆)ₘimidazolyl(Aa)ₜ-, —(CR₅R₆)ₘ-morpholino-(Aa)ₜ-, —(CR₅R₆)ₘ-piperazino-(Aa)ₜ-, —(CR₅R₆)ₘ—N-methylpiperazino-(Aa)ₜ-, —K(CR₅R₆)ₘ(Aa)r(CR₇R₈)ₙ(OCH₂CH₂)ₜ—, —K(CR₅R₆)ₘ(CR₇R₈)ₙ(Aa)ᵣ(OCH₂CH₂)ₜ—, —K(Aa)ᵣ-(CR₅R₆)ₘ(CR₇R₈)ₙ(OCH₂CH₂)ₜ—, —K(CR₅R₆)ₘ(CR₇R₈)ₙ(OCH₂CH₂)ᵣ(Aa)ₜ-, —K(CR₅R₆)ₘ—(CR₇═CR₈)(CR₉R₁₀)ₙ(Aa)ₜ(OCH₂CH₂)ᵣ—, —K(CR₅R₆)ₘ(NR₁₁CO)(Aa)ₜ(CR₉R₁₀)ₙ(OCH₂CH₂)ᵣ—, —K(CR₅R₆)ₘ(Aa)ₜ(NR₁₁CO)(CR₉R₁₀)ₙ(OCH₂CH₂)ᵣ—, —K(CR₅R₆)ₘ(OCO)(Aa)ₜ(CR₉R₁₀)ₙ—(OCH₂CH₂)ᵣ—, —K(CR₅R₆)ₘ(OCNR₇)(Aa)ₜ(CR₉R₁₀)ₙ(OCH₂C H₂)ᵣ—, —K(CR₅R₆)ₘ(CO)(Aa)ₜ-(CR₉R₁₀)ₙ(OCH₂CH₂)ᵣ—, —K(CR₅R₆)ₘ(NR₁₁CO)(Aa)ₜ(CR₉R₁₀)ₙ(OCH₂CH₂)ᵣ—, —K(CR₅R₆)ₘ—(OCO)(Aa)ₜ(CR₉R₁₀)ₙ(OCH₂CH₂)ᵣ—, —K(CR₅R₆)ₘ(OCNR₇)(Aa)ₜ(CR₉R₁₀)ₙ(OCH₂CH₂)ᵣ—, —K—(CR₅R₆)ₘ(CO)(Aa)ₜ(CR₉R₁₀)ₙ(OCH₂CH₂)ᵣ—, —K(CR₅R₆)ₘ-phenyl-CO(Aa)ₜ(R₇R₈)ₙ—, —K—(CR₅R₆)ₘ-furyl-CO(Aa)ₜ(CR₇R₈)ₙ—, —K(CR₅R₆)ₘ-oxazolyl-CO(Aa)ₜ(CR₇R₈)ₙ—, —K(CR₅R₆)ₘ-thiazolyl-CO(Aa)ₜ-(CR₇R₈)ₙ—, —K(CR₅R₆)ₜ-thienyl-CO(CR₇R₈)ₙ, —K(CR₅R₆)imidazolyl-CO—(CR₇R₈)ₙ—, —K(CR₅R₆)ₜmorpholino-CO(Aa)ₜ(CR₇R₈)ₙ—, —K(CR₅R₆)ₜpiperazino-CO(Aa)ₜ-(CR₇R₈)ₙ—, —K(CR₅R₆)ₜ—N-methylpiperazinCO(Aa)ₜ(CR₇R₈)ₙ—, —K(CR₅R₆)ₘ(Aa)ₜphenyl, —K—(CR₅R₆)ₘ-(Aa)ₜfuryl-, —K(CR₅R₆)ₘ-oxazolyl(Aa)ₜ-, —K(CR₅R₆)ₘ-thiazolyl(Aa)ₜ-, —K(CR₅R₆)ₘ-thienyl-(Aa)ₜ-, —K(CR₅R₆)ₘ-imidazolyl(Aa)ₜ-, —K(CR₅R₆)ₘ-morpholino(Aa)ₜ-, —K(CR₅R₆)ₘ-piperazino-(Aa)ₜG, —K(CR₅R₆)ₘN-methylpiperazino(Aa)ₜ-; wherein m, Aa, m, and n are described above; t and r are 0-100 independently; R₃, R₄, R₅, R₆, R₇, and R₈ are independently chosen from H; halide; C₁˜C₈ alkyl; C₂˜C₈ aryl, alkenyl, alkynyl, ether, ester, amine or amide, which optionally substituted by one or more halide, CN, NR₁R₂, CF₃, OR₁, Aryl, heterocycle, S(O)R₁, SO₂R₁, —CO₂H—, —SO₃H, —OR₁, —CO₂R₁, —CONR₁, —PO₂R₁R₂, —PO₃H or P(O)R₁R₂R₃; K is NR₁, —SS—, —C(═O)—, —C(═O)NH—, —C(═O)O—, —C═NH—O—, —C═N—NH—, —C(═O)NH—NH—, O, S, Se, B, Het (heterocyclic or heteroaromatic ring having C₃-C₈), or peptides containing 1-20 amino acids;

Additionally L₁ and L₂ may independently contain one of the following hydrophilic structures:

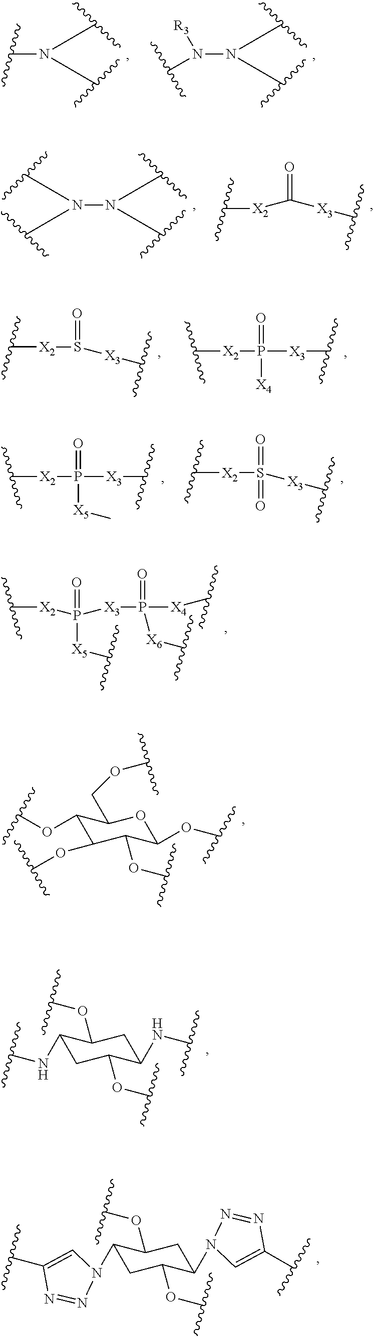

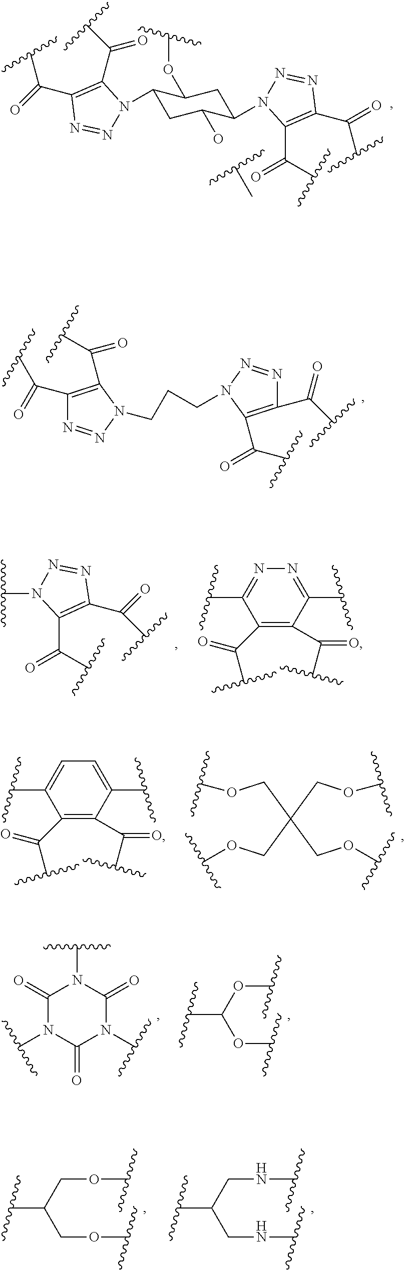

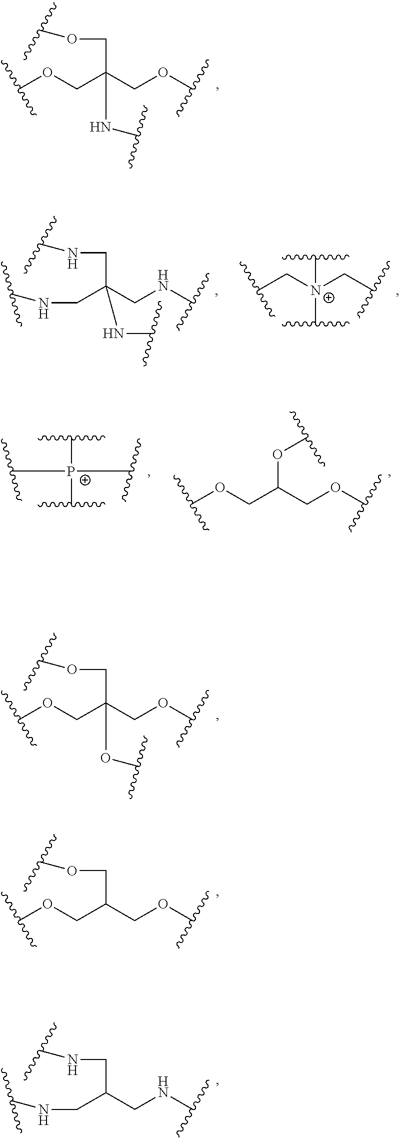

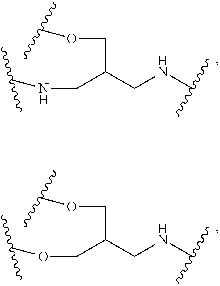

wherein [symbol] is the site of linkage; X₂, X₃, X₄, X₅, or X₆, are independently selected from NH; NHNH; N(R₃); N(R₃)N(R_{3′}); O; S; C₁-C₆ alkyl; C₂-C₆ heteroalkyl, alkylcycloalkyl, or heterocycloalkyl; C₃-C₈ aryl, Ar-alkyl, heterocyclic, carbocyclic, cycloalkyl, heteroalkylcycloalkyl, alkylcarbonyl, or heteroaryl; or 1˜8 amino acids; Wherein R₃ and R_{3′} are independently H; C₁-C₈ alkyl; C₂-C₈ hetero-alkyl, alkylcycloalkyl, or heterocycloalkyl; C₃-C₈ aryl, Ar-alkyl, heterocyclic, carbocyclic, cycloalkyl, heteroalkylcycloalkyl, alkylcarbonyl, or heteroaryl; or C₂-C₈ esters, ether, or amide; or polyethyleneoxy unit having formula (OCH₂CH₂)ₚ or (OCH₂CH(CH₃))ₚ, wherein p is an integer from 0 to about 5000, or combination above thereof;

More preferably, R₁, L₁, or L₂, are independently linear alkyl having from 1-6 carbon atoms, or polyethyleneoxy unit having formula (OCH₂CH₂)ₚ, p=1˜5000, or a peptide containing 1˜4 units of aminoacids (L or D form), or combination above.

In addition, X, Y, L₁, L₂, Z₁ or Z₂ may independently be composed of one or more following components as shown below:

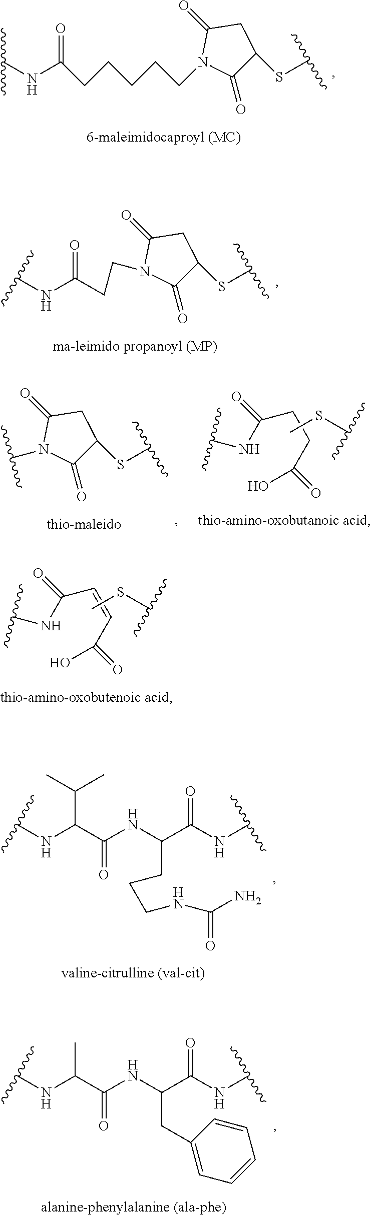

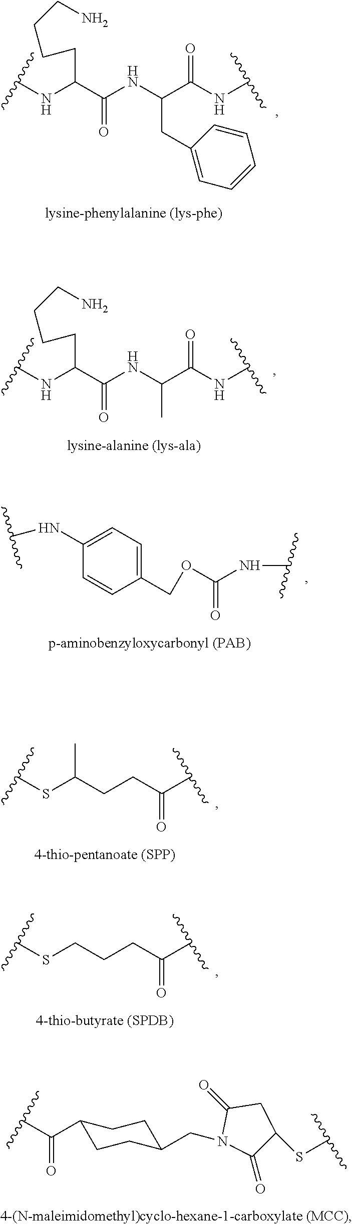

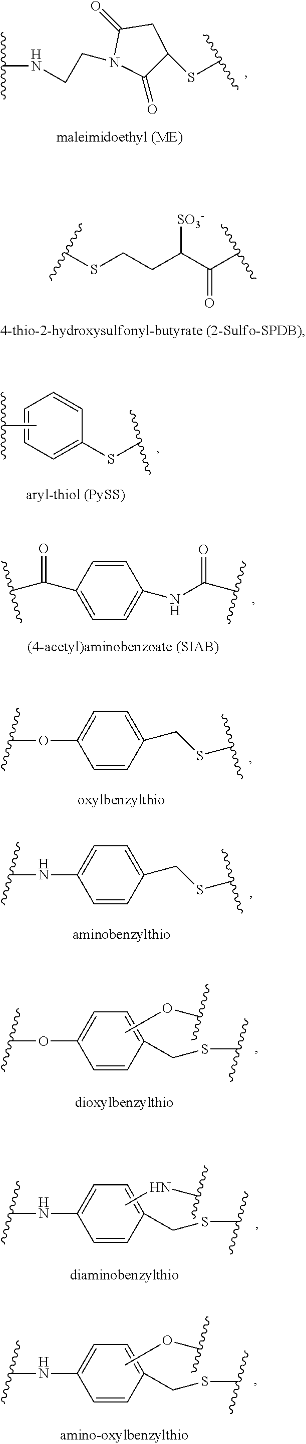

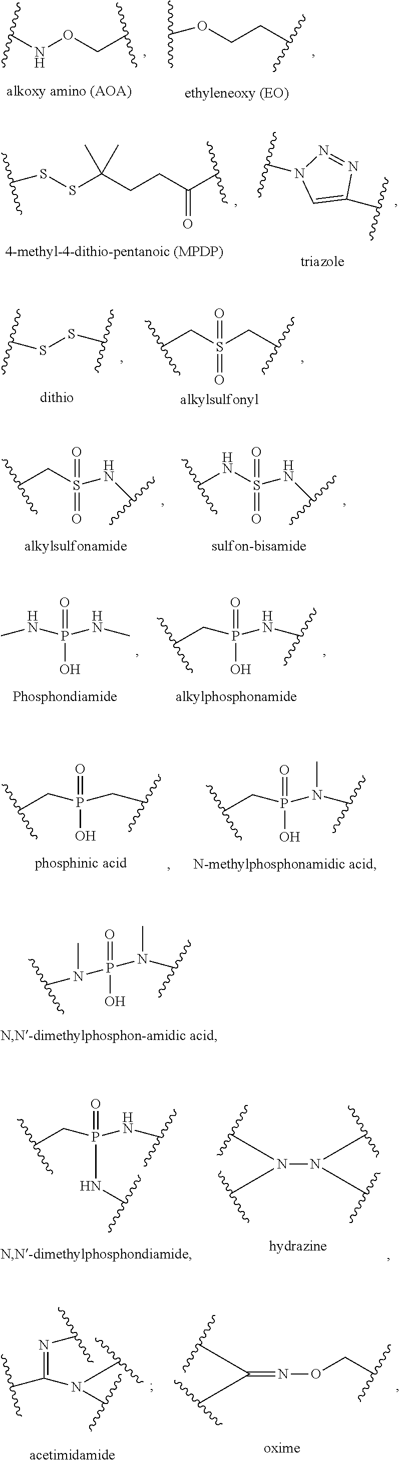

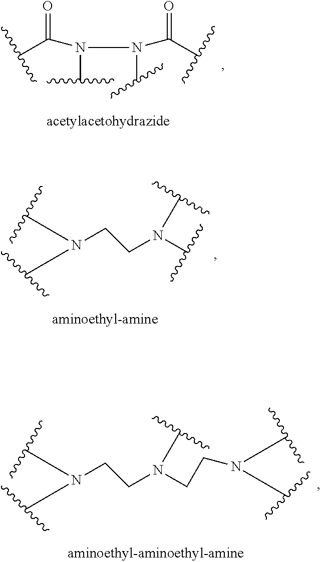 [SMILES: *C(=O)N(*)N(*)C(*)=O ; *N(*)CCN(*)* ; *N(*)CCN(*)CCN(*)*]

and L- or D-, natural or unnatural peptides containing 1-20 amino acids; wherein a connecting bond in the middle of atoms means that it can connect either neighbor carbon atom bonds; wavery line is the site wherein another bond can be connected to;

Alternatively, X, Y, L₁, L₂, Z₁, or Z₂, can be independently absent, but L₁ and Z₁, or L₂ and Z₂ may not be absent at the same time.

Preferably bis-linkage of the conjugate is further represented by Formula (I-a), (I-b), (I-c), (I-d), (I-e), (I-f), (I-g), (I-h), (I-i), (I-j), (I-k), (I-m), (I-n), (I-o), (I-p), (I-q), (I-r), (I-s), (I-t), (I-u), (I-v), and (I-w) below:

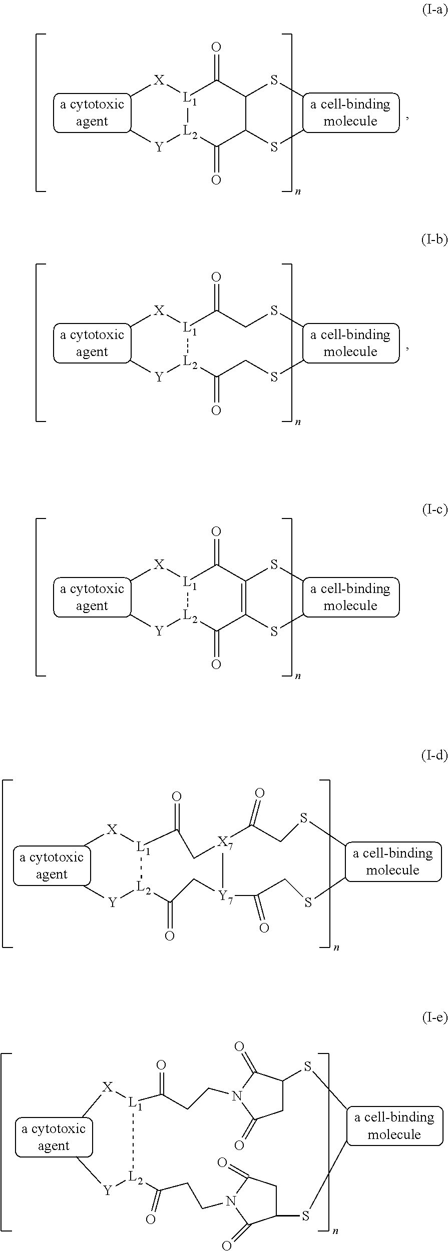

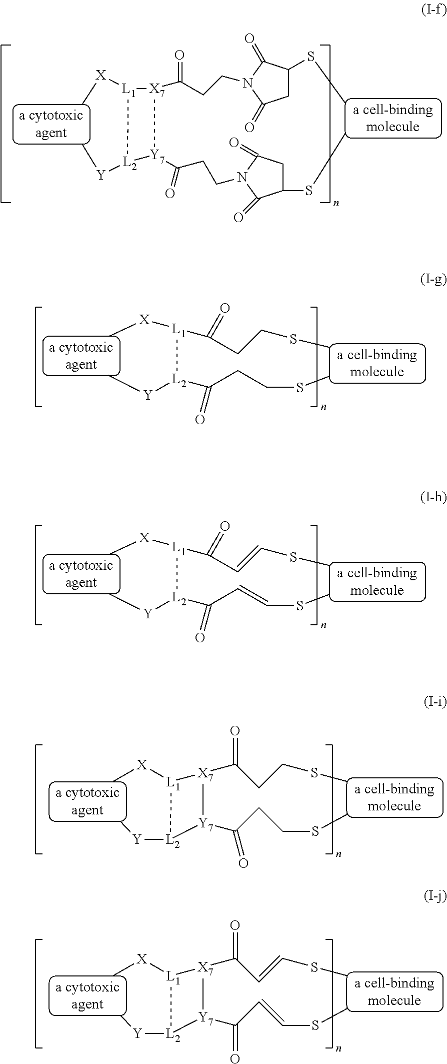

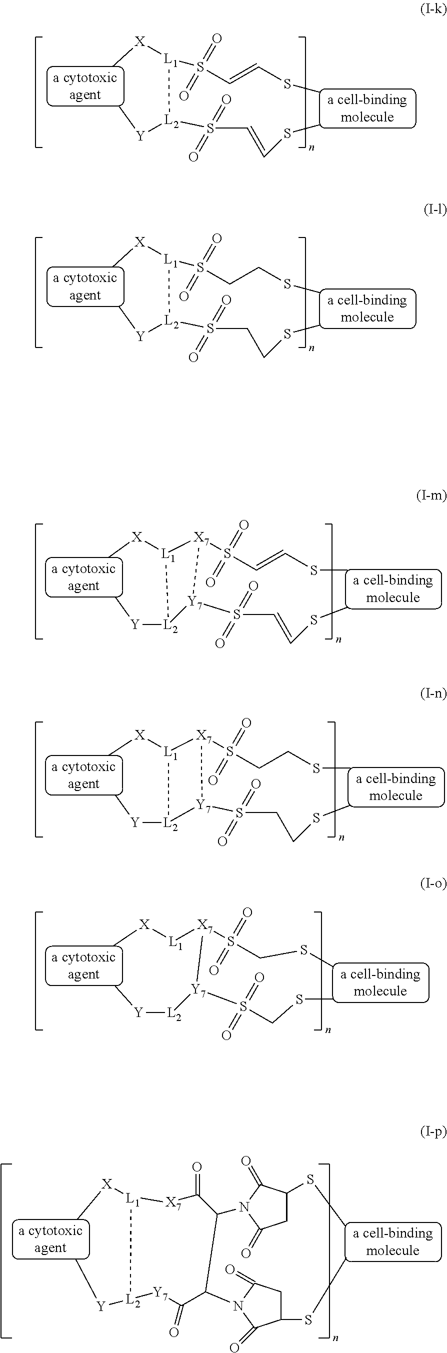

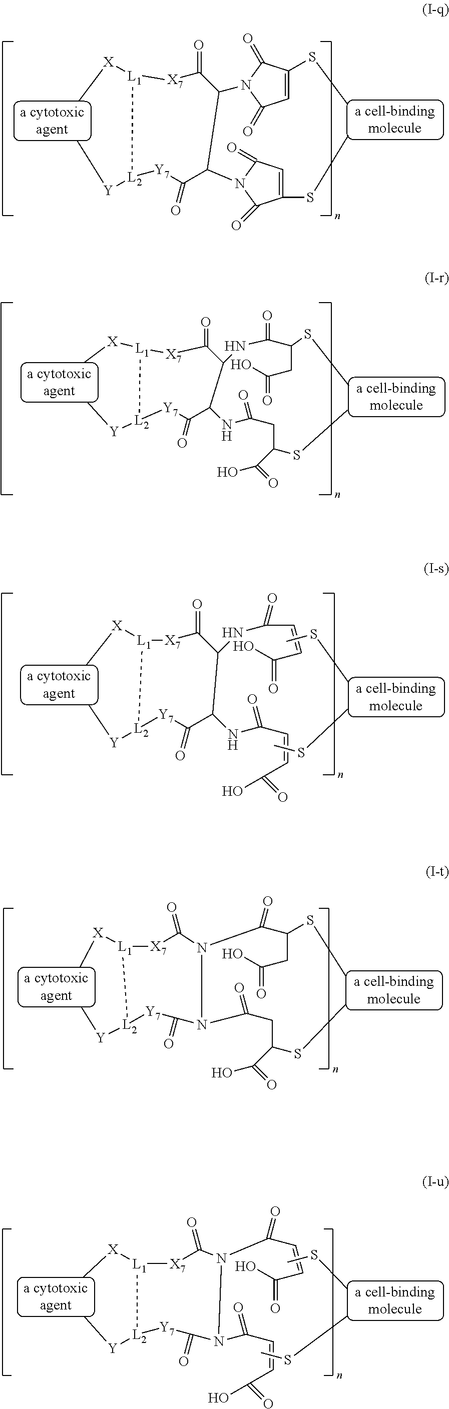

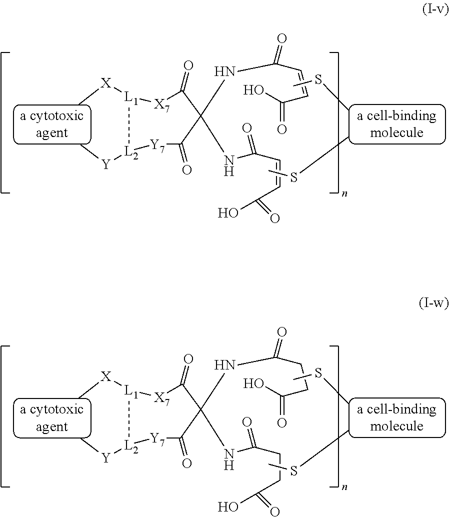

wherein X₇ and Y₇ are independently CH, CH₂, NH, O, S, NHNH, N(R₁), and N; the chemical bond in the middle of two atoms means it can link either adjoining two atoms; “-----”, X, Y, R₁, n, L₁ and L₂ are the same described above; the cytotoxic agent is the same cytotoxic molecule described above.

In a more preferable aspect, X and Y are independently a group of amino, hydroxyl, diamino, amino-hydroxyl, dihydroxyl, carboxyl, aldehyde, hydrazine, thiol, phosphate or sulfonyl on an aromatic ring.

The Preparation of the Conjugates of Drugs to a Cell Binding Molecules Via a Bis-Linkage

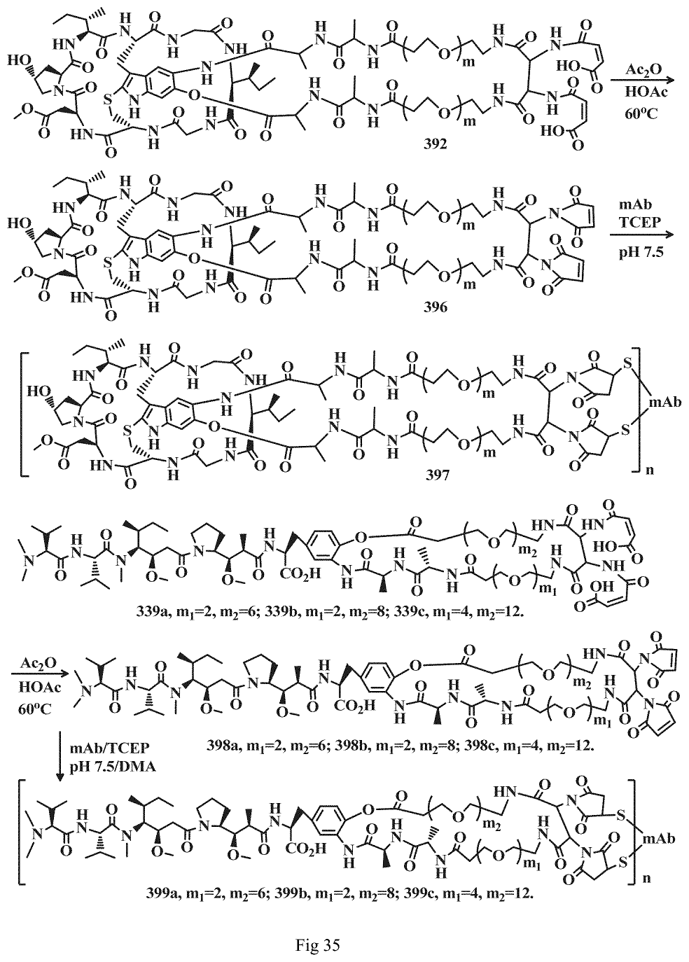

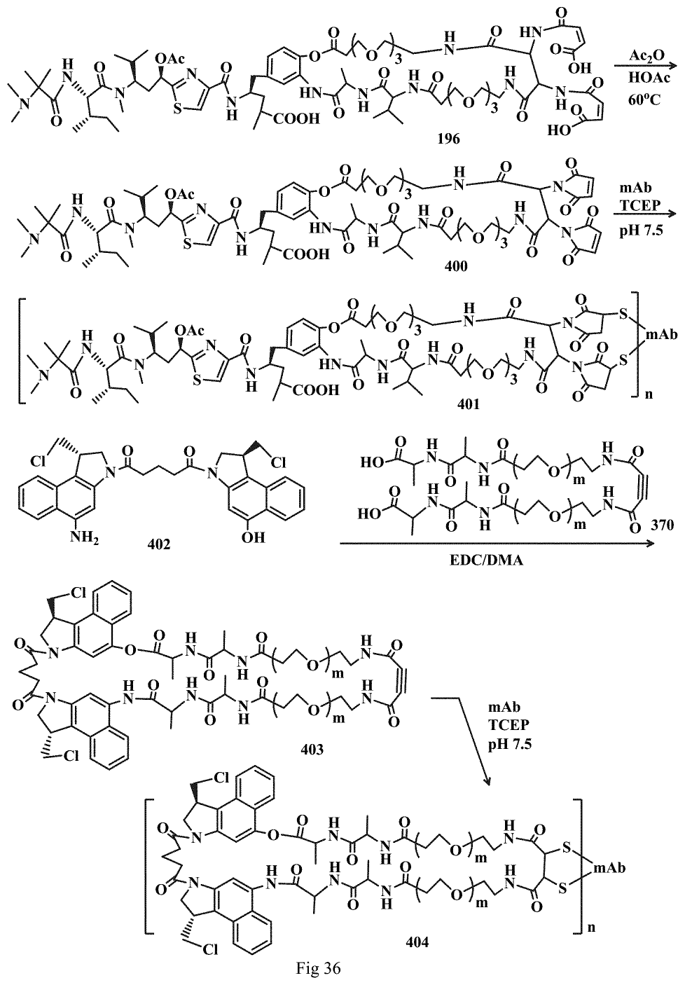

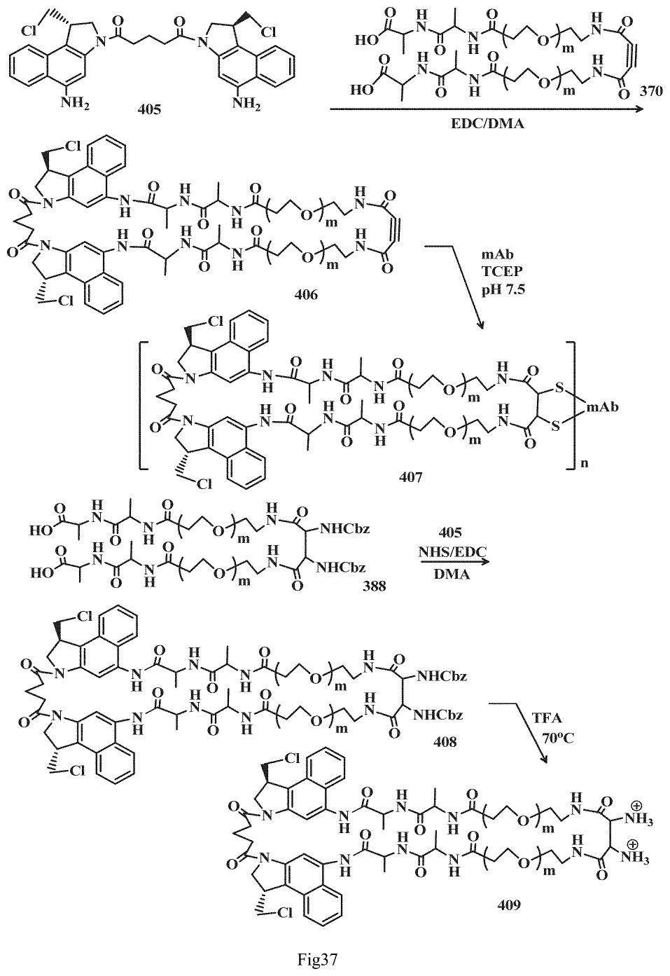

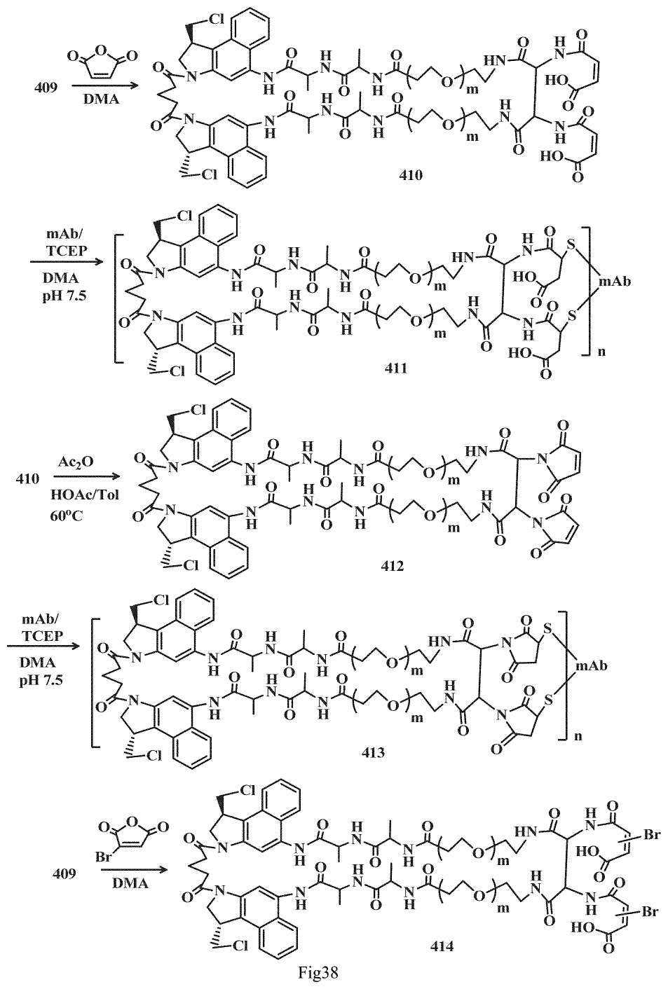

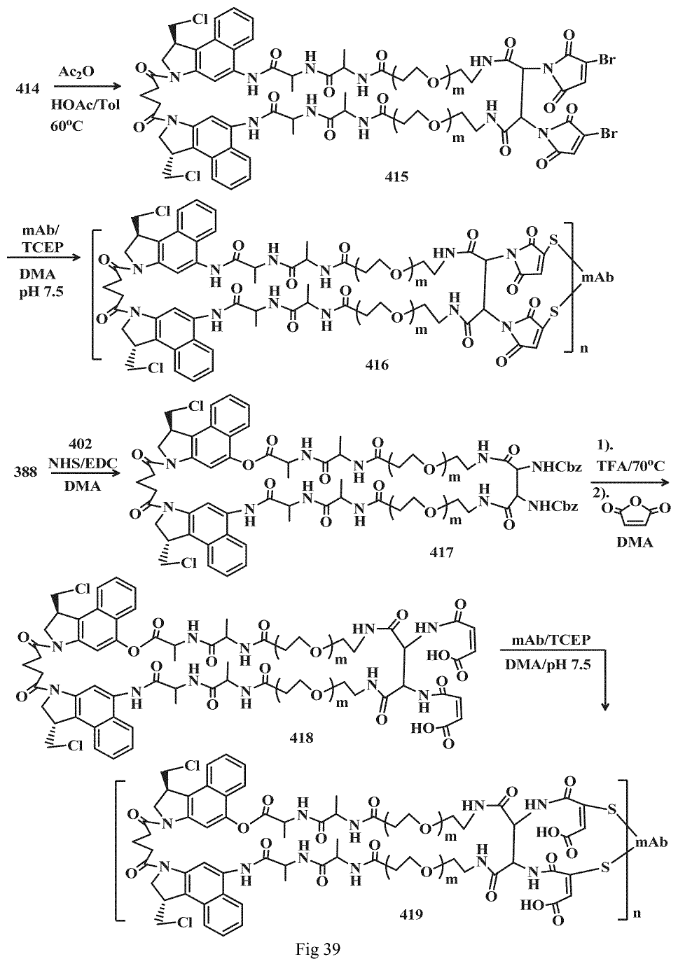

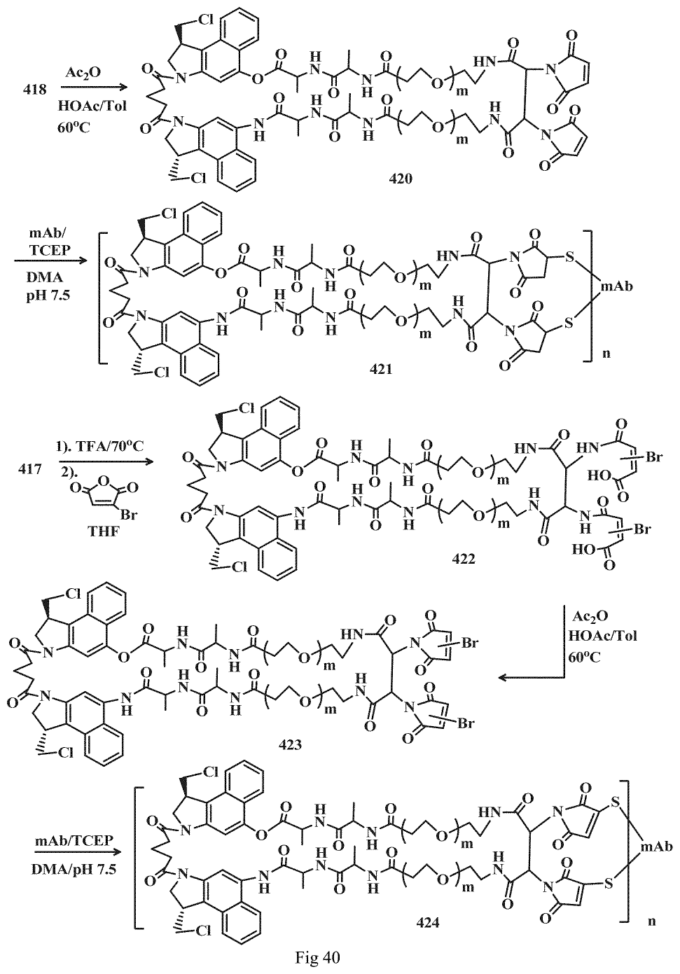

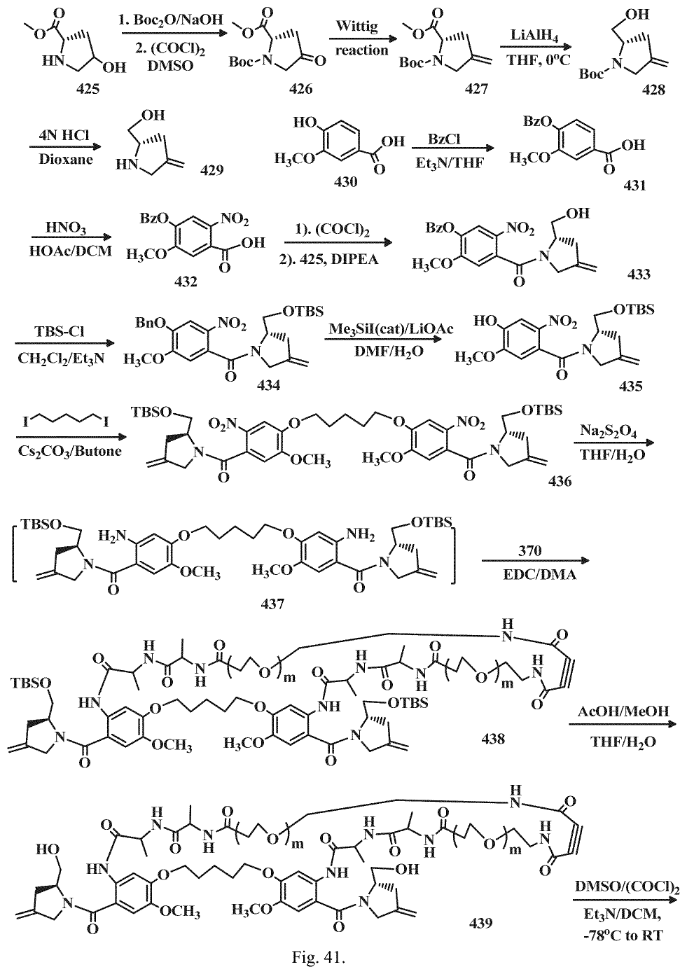

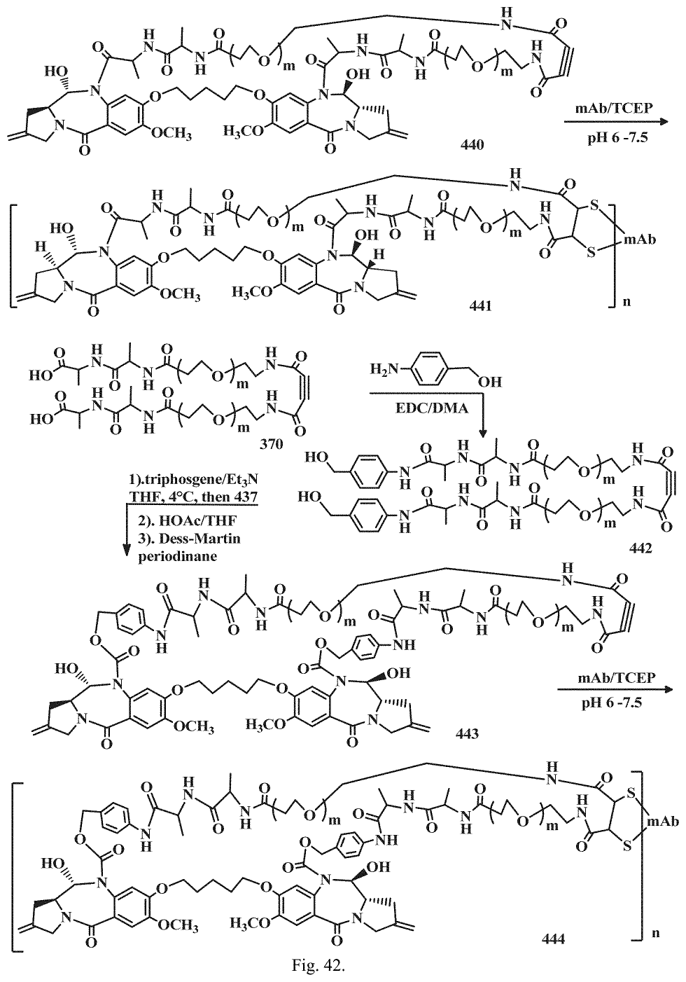

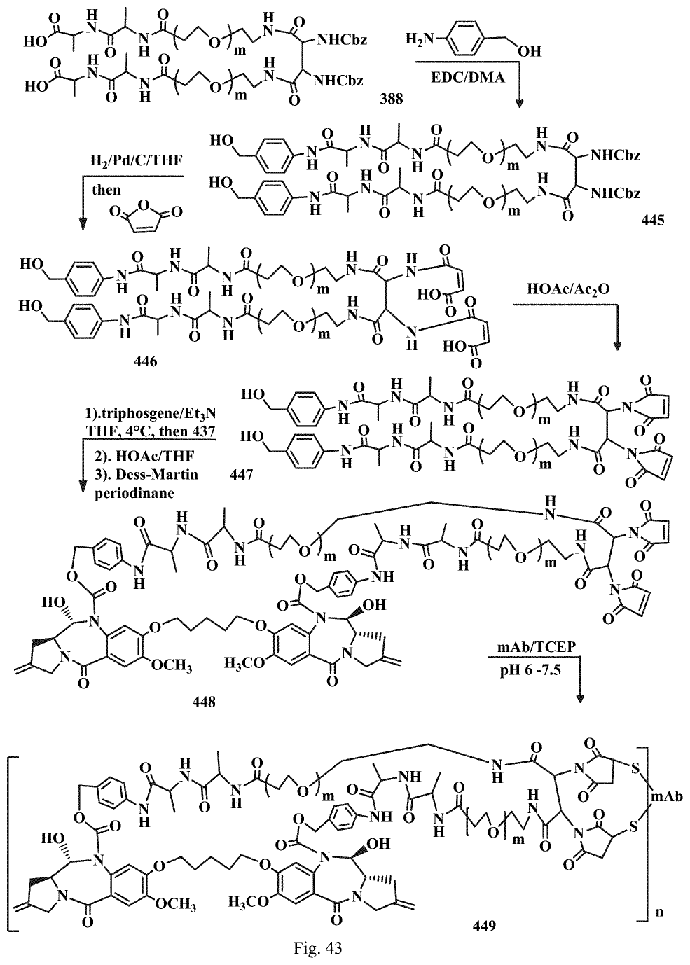

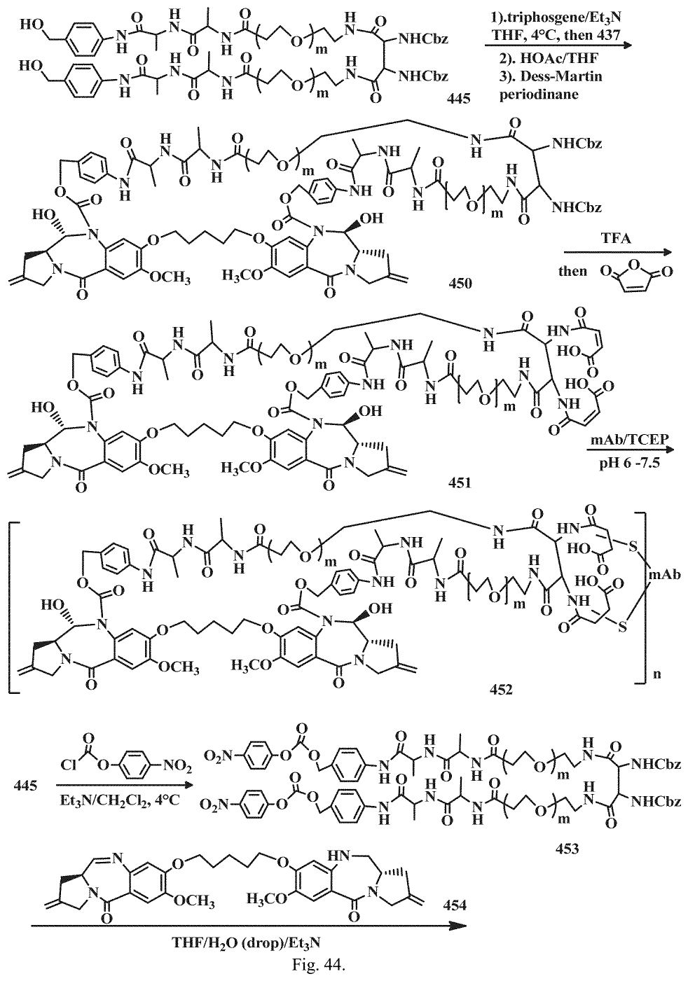

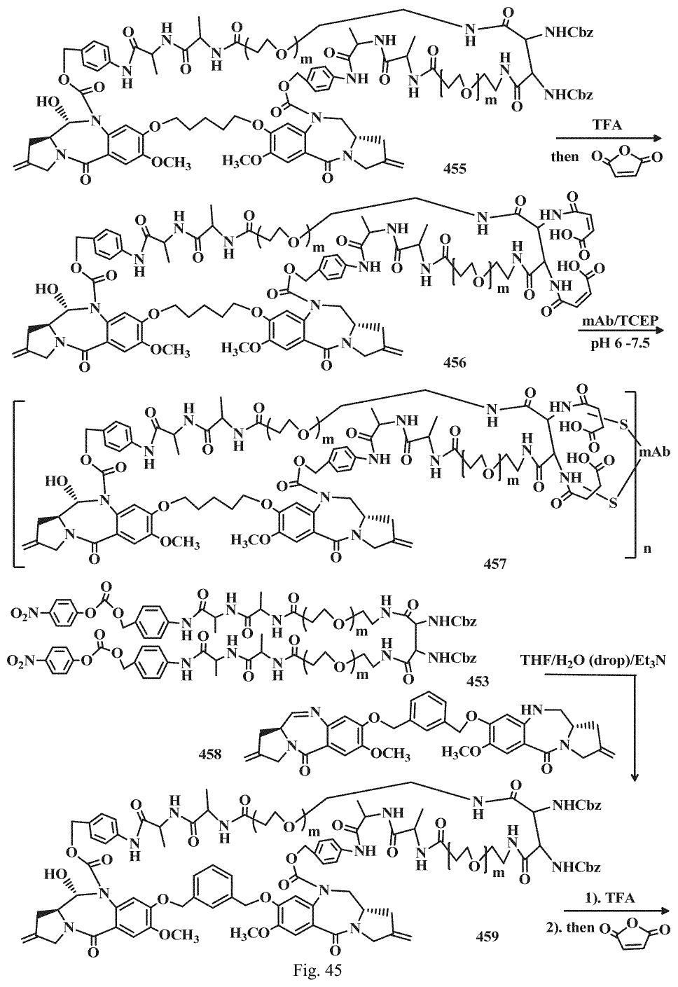

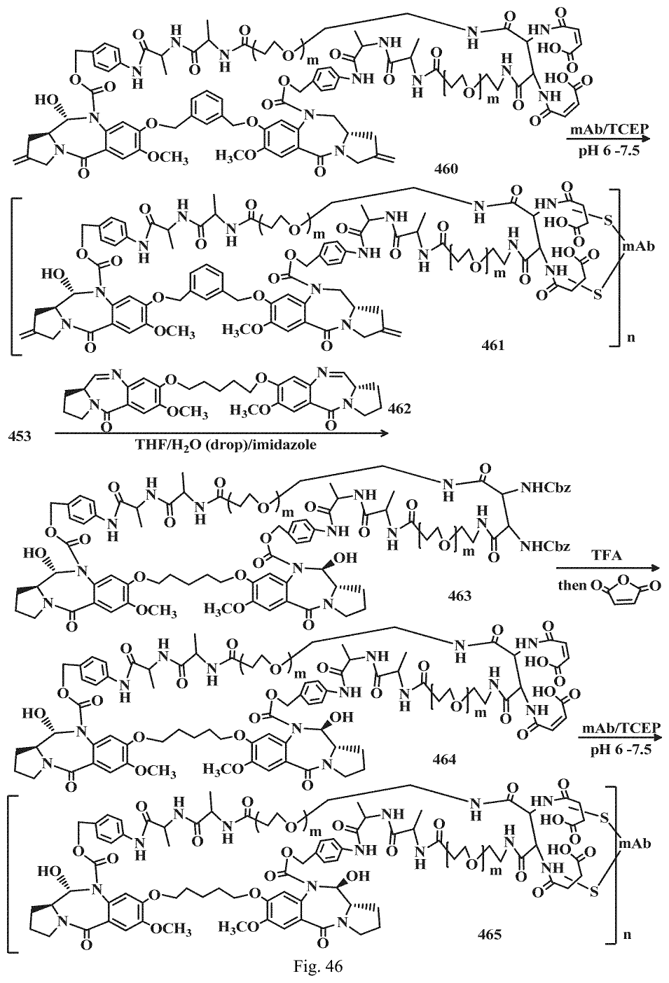

The preparation of the conjugates of drugs to a cell binding molecules of the present invention and the synthetic routes to produce the conjugates via bis-linkage are shown in FIGS. 1-46.

In an aspect, this invention provides a readily-reactive bis-linker containing a cytotoxic molecule of Formula (II) below, wherein two or more residues of the cell-binding molecule can simultaneously or sequentially react it to form Formula (I).

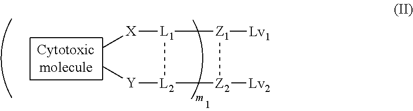

wherein:

“ [symbol] ” represents a single bond;

“ [symbol] ” is optionally either a single bond, or a double bond, or a triple bond, or can optionally be absent;

It provided that when ----- represents a triple bond, both Lv₁ and Lv₂ are absent;

Cytotoxic molecule in the frame, m₁, X, Y, L₁, L₂, Z₁, and Z₂ are defined the same as in Formula (I);

Lv₁ and Lv₂ represent the same or different leaving group that can be reacted with a thiol, amine, carboxylic acid, selenol, phenol or hydroxyl group on a cell-binding molecule. Lv₁ and Lv₂ are independently selected from OH; F; Cl; Br; I; nitrophenol; N-hydroxysuccinimide (NHS); phenol; dinitrophenol; pentafluorophenol; tetrafluorophenol; difluorophenol; mono-fluorophenol; pentachlorophenol; triflate; imidazole; dichlorophenol; tetrachlorophenol; 1-hydroxybenzotriazole; tosylate; mesylate; 2-ethyl-5-phenylisoxazolium-3′-sulfonate, anhydrides formed its self, or formed with the other anhydride, e.g. acetyl anhydride, formyl anhydride; or an intermediate molecule generated with a condensation reagent for peptide coupling reactions, or for Mitsunobu reactions. The examples of condensation reagents are: EDC (N-(3-Dimethylaminopropyl)-N′-ethylcarbodiimide), DCC (Dicyclohexyl-carbodiimide), N,N′-Diisopropylcarbodiimide (DIC), N-Cyclohexyl-N′-(2-morpholino-ethyl)carbodiimide metho-p-toluenesulfonate (CMC, or CME-CDI), 1,1′-Carbonyldiimi-dazole (CDI), TBTU (O-(Benzotriazol-1-yl)-N,N,N′,N′-tetramethyluronium tetrafluoroborate), N,N,N′,N′-Tetramethyl-O-(1H-benzotriazol-1-yl)-uronium hexafluorophosphate (HBTU), (Benzotriazol-1-yloxy)tris(dimethylamino)-phosphonium hexafluorophosphate (BOP), (Benzotriazol-1-yloxy)tripyrrolidinophosphonium hexafluorophosphate (PyBOP), Diethyl cyanophosphonate (DEPC), Chloro-N,N,N′,N′-tetramethylformamidiniumhexafluorophosphate, 1-[Bis(dimethylamino)methylene]-1H-1,2,3-triazolo[4,5-b]pyridinium 3-oxid hexafluorophosphate (HATU), 1-[(Dimethylami-no)(morpholino)methylene]-1H-[1,2,3]triazolo[4,5-b]pyridine-1-ium 3-oxide hexafluoro-phosphate (HDMA), 2-Chloro-1,3-dimethyl-imidazolidinium hexafluorophosphate (CIP), Chlorotripyrrolidinophosphonium hexafluorophosphate (PyCloP), Fluoro-N,N,N′,N′-bis(tetramethylene)formamidinium hexafluorophosphate (BTFFH), N,N,N′,N′-Tetramethyl-S-(1-oxido-2-pyridyl)thiuronium hexafluorophosphate, 0-(2-Oxo-1 (2H)pyridyl)-N,N,N′,N′-tetramethyluronium tetrafluoroborate (TPTU), S-(1-Oxido-2-pyridyl)-N,N,N′,N′-tetramethylthiuronium tetrafluoroborate, O-[(Ethoxycarbonyl)-cyanomethylenamino]-N,N,N′,N′-tetramethyluronium hexafluorophosphate (HOTU), (1-Cyano-2-ethoxy-2-oxoethylidenaminooxy) dimethylamino-morpholino-carbenium hexafluorophosphate (COMU), O-(Benzotriazol-1-yl)-N,N,N′,N′-bis(tetramethylene)uronium hexafluorophosphate (HBPyU), N-Benzyl-N′-cyclohexyl-carbodiimide (with, or without polymer-bound), Dipyrrolidino(N-succinimidyl-oxy)carbenium hexafluoro-phosphate (HSPyU), Chlorodipyrrolidinocarbenium hexafluorophosphate (PyC1U), 2-Chloro-1,3-dimethylimidazolidinium tetrafluoroborate (CIB), (Benzotriazol-1-yloxy)dipiperidino-carbenium hexafluorophosphate (HBPipU), O-(6-Chlorobenzotriazol-1-yl)-N,N,N′,N′-tetramethyluronium tetrafluoroborate (TCTU), Bromotris(dimethylamino)-phosphonium hexafluorophosphate (BroP), Propylphosphonic anhydride (PPACA, T3P®), 2-Morpholinoethyl isocyanide (MEI), N,N,N′,N′-Tetramethyl-O—(N-succinimidyl)uronium hexafluorophosphate (HSTU), 2-Bromo-1-ethyl-pyridinium tetrafluoroborate (BEP), O-[(Ethoxycarbonyl)cyano-methylenamino]-N,N,N′,N′-tetra-methyluronium tetrafluoroborate (TOTU), 4-(4,6-Dimethoxy-1,3,5-triazin-2-yl)-4-methylmorpholiniumchloride (MM™, DMTMM), N,N,N′,N′-Tetramethyl-O—(N-succinimidyl)uronium tetrafluoroborate (TSTU), O-(3,4-Dihydro-4-oxo-1,2,3-benzotriazin-3-yl)-N,N,N′,N′-tetramethyluronium tetrafluoro-borate (TDBTU), 1,1′-(Azodicarbonyl)-dipiperidine (ADD), Di-(4-chlorobenzyl)azodicarboxylate (DCAD), Di-tert-butyl azodicarboxylate (DBAD), Diisopropyl azodicarboxylate (DIAD), Diethyl azodicarboxylate (DEAD). In addition, Lv₁ and Lv₂ can be an anhydride, formed by acid themselves or formed with other C₁˜C₈ acid anhydrides;

Preferably Lv₁ and Lv₂ are independently selected from, a halide (e.g., fluoride, chloride, bromide, and iodide), methanesulfonyl (mesyl), toluenesulfonyl (tosyl), trifluoromethyl-sulfonyl (triflate), trifluoromethylsulfonate, nitrophenoxyl, N-succinimidyloxyl (NHS), phenoxyl; dinitrophenoxyl; pentafluorophenoxyl, tetrafluorophenoxyl, trifluorophenoxyl, difluorophenoxyl, monofluorophenoxyl, pentachlorophenoxyl, 1H-imidazole-1-yl, chlorophenoxyl, dichlorophenoxyl, trichlorophenoxyl, tetrachlorophenoxyl, N-(benzotriazol-yl)oxyl, 2-ethyl-5-phenylisoxazolium-3′-sulfonyl, phenyloxadiazole-sulfonyl (-sulfone-ODA), 2-ethyl-5-phenylisoxazolium-yl, phenyloxadiazol-yl (ODA), oxadiazol-yl, unsaturated carbon (a double or a triple bond between carbon-carbon, carbon-nitrogen, carbon-sulfur, carbon-phosphorus, sulfur-nitrogen, phosphorus-nitrogen, oxygen-nitrogen, or carbon-oxygen), or one of the following structure:

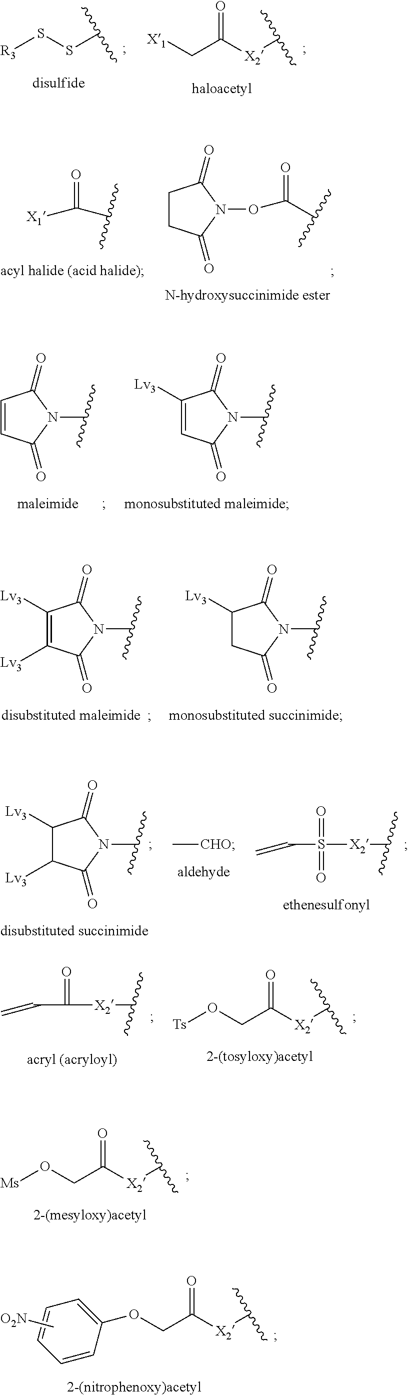

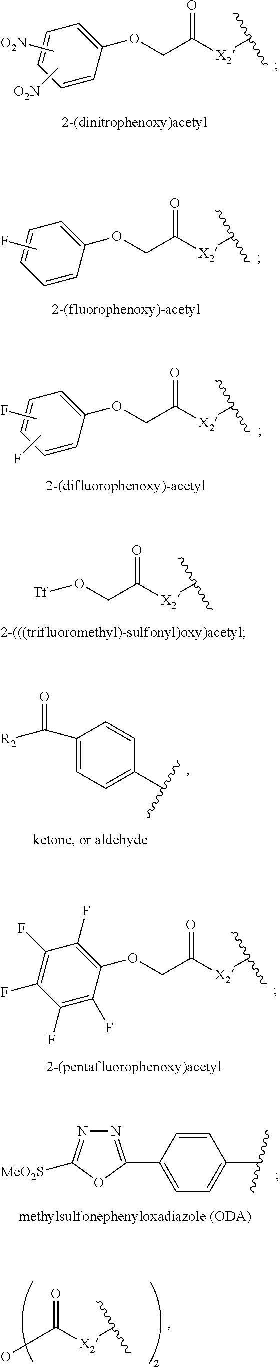

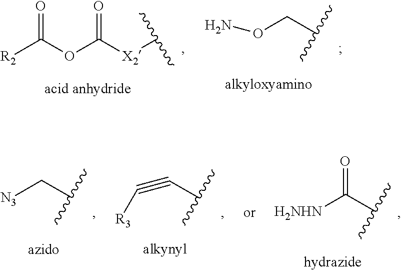

wherein X₁′ is F, Cl, Br, I or Lv₃; X₂′ is O, NH, N(R₁), or CH₂; R₃ is independently H, aromatic, heteroaromatic, or aromatic group wherein one or several H atoms are replaced independently by —R₁, -halogen, —OR₁, —SR₁, —NR₁R₂, —NO₂, —S(O)R₁, —S(O)₂R₁, or —COOR¹; Lv₃ is a leaving group selected from F, Cl, Br, I, nitrophenol; N-hydroxysuccinimide (NHS); phenol; dinitrophenol; pentafluorophenol; tetrafluorophenol; difluorophenol; monofluorophenol; pentachlorophenol; triflate; imidazole; dichlorophenol; tetrachlorophenol; 1-hydroxybenzotriazole; tosylate; mesylate; 2-ethyl-5-phenylisoxazolium-3′-sulfonate, anhydrides formed its self, or formed with the other anhydride, e.g. acetyl anhydride, formyl anhydride; or an intermediate molecule generated with a condensation reagent for peptide coupling reactions or for Mitsunobu reactions;

R₁ and R₂ are independently selected from H, C₁-C₈ alkyl, C₂-C₈ alkenyl, heteroalkyl, alkylcycloalkyl, or heterocycloalkyl; C₃-C₈ aryl, Ar-alkyl, heterocyclic, carbocyclic, cycloalkyl, heteroalkylcycloalkyl, alkylcarbonyl, or heteroaryl, or C₂-C₈ esters, ether, or amide; or peptides containing 1-8 amino acids; or polyethyleneoxy unit having formula (OCH₂CH₂)ₚ or (OCH₂CH(CH₃))ₚ, wherein p is an integer from 0 to about 5000, or combination of above groups thereof;

In addition, the functional groups, X or Y, which enables linkage of a drug or a cytotoxic agent, preferably include groups that enable linkage via a disulfide, thioether, thioester, peptide, hydrazone, ester, carbamate, carbonate, alkoxime or an amide bond. Such functional groups include, but are not limited to, thiol, disulfide, amino, carboxyl, aldehydes, ketone, maleimido, haloacetyl, hydrazines, alkoxyamino, and/or hydroxy;

Preferably bis-linkage of the conjugate is further represented by Formula (II-a), (II-b), (II-c), (II-d), (II-e), (II-f), (II-g), (II-h), (II-i), (II-j), (II-k), (II-m), (II-n), (II-o), (II-q), (II-r), (II-s), (II-t), (II-u), (II-v), (II-w), (II-x), (II-y), (II-z), (II-a1), (II-a2), (II-a3), and (II-a4):

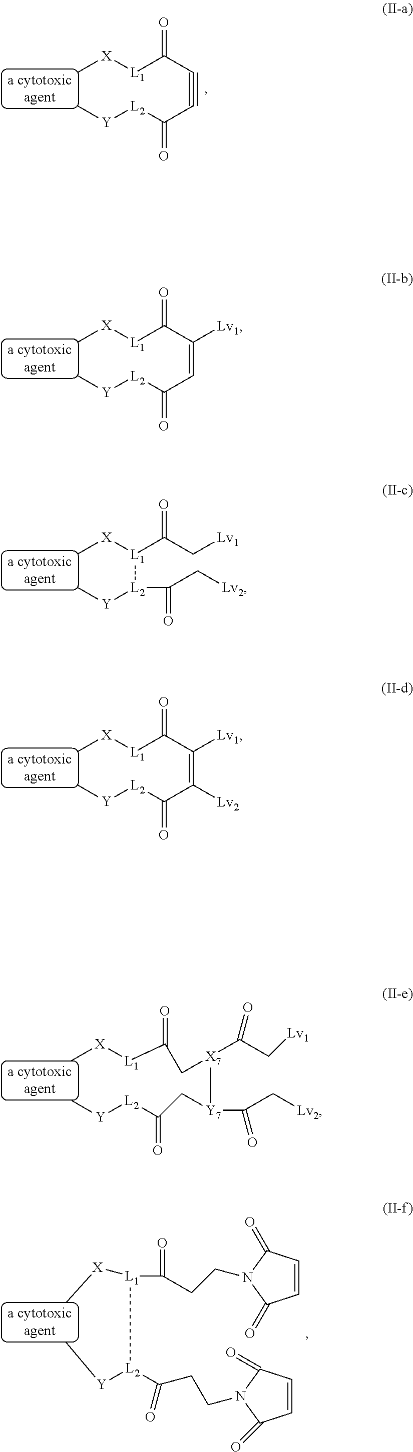

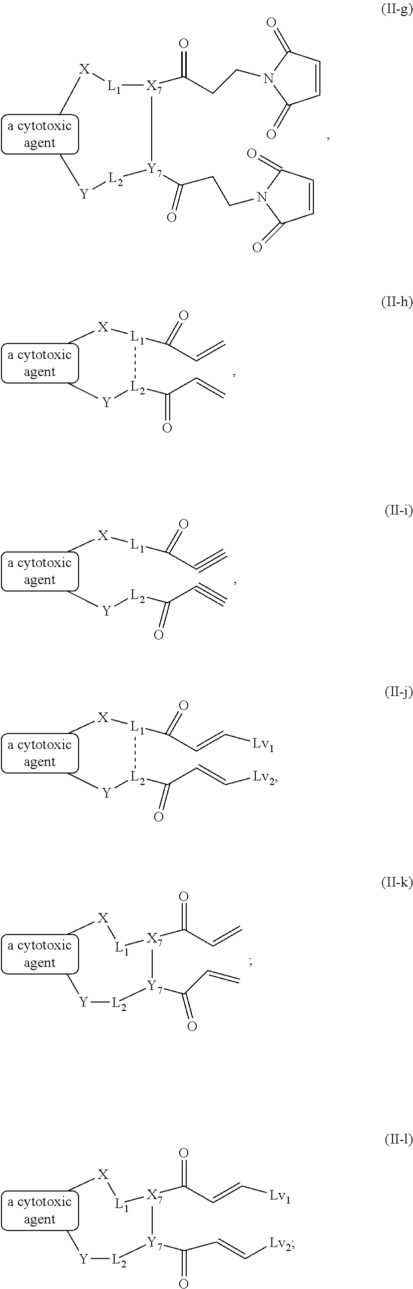

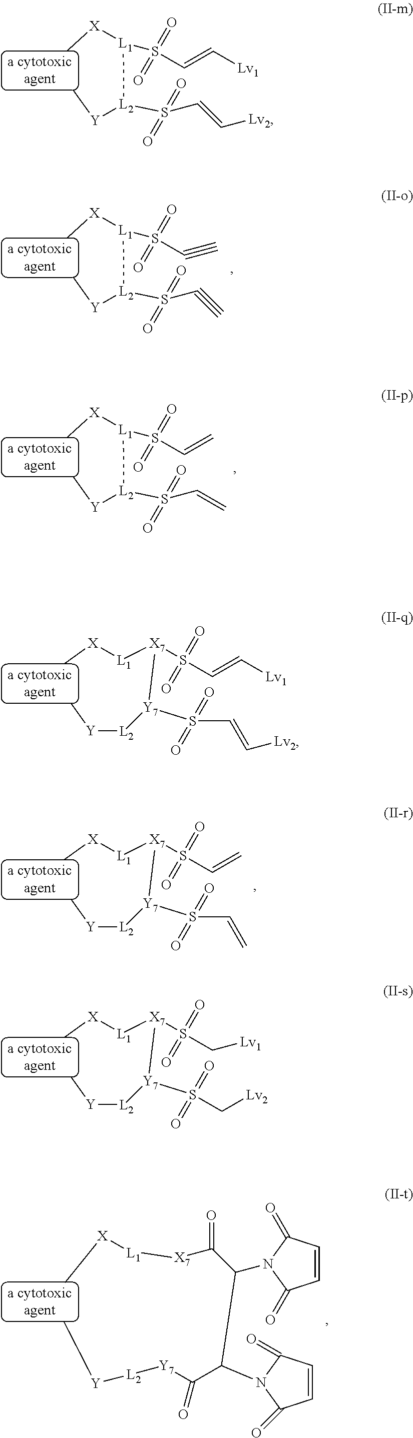

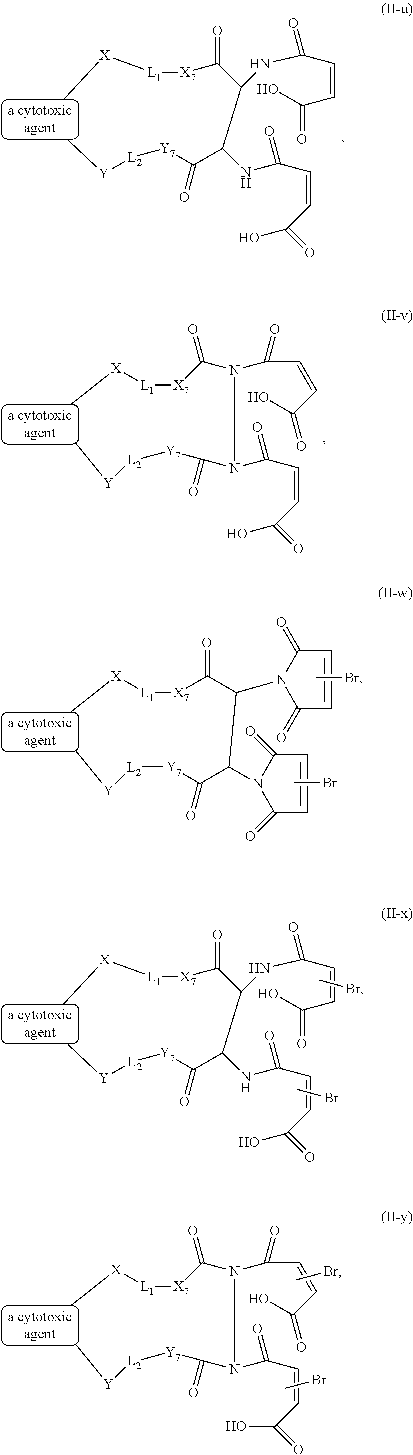

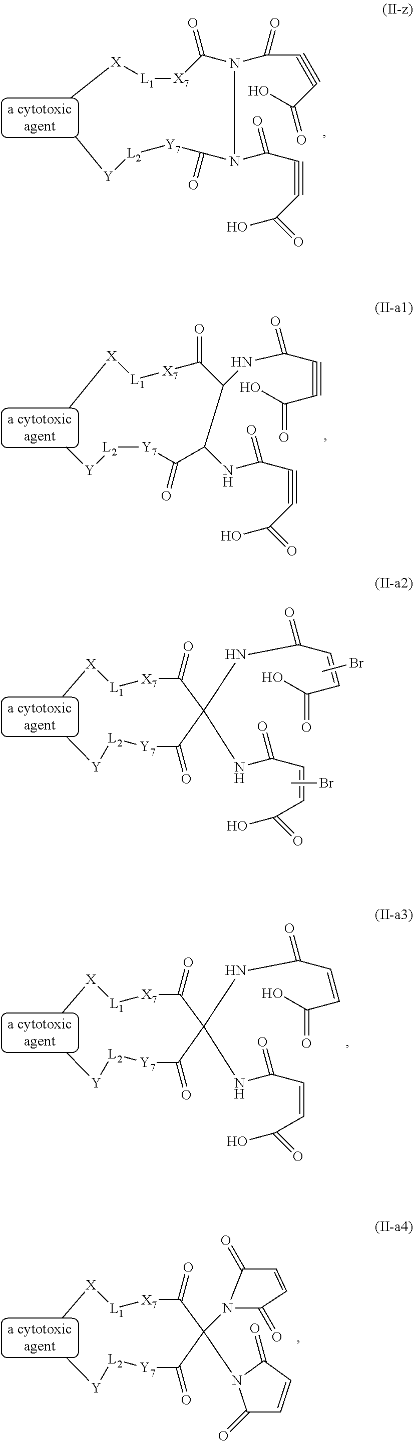

wherein X₇ and Y₇ are independently CH, CH₂, NH, O, S, NHNH, N(R₁), and N; X, Y, R₁, n, “-----”, L₁ and L₂ are the same described above; a chemical bond in the middle of two atoms means it can link either adjoining two atoms; R₁, X, Y, n, L₁, L₂, Lv₁ and Lv₂ are the same described above. Preferably Lv1 and Lv2 are independently selected from Cl, Br, I, methanesulfonyl (mesyl), toluenesulfonyl (tosyl), trifluoromethyl-sulfonyl (triflate), trifluoromethylsulfonate, and nitrophenoxyl.

In another aspect, this invention provides a readily-reactive bis-linker having conjugated to a cell-binding agent/molecule of Formula (III) below, wherein two or more function groups of a cytotoxic molecule can react it simultaneously or sequentially to form Formula (I):

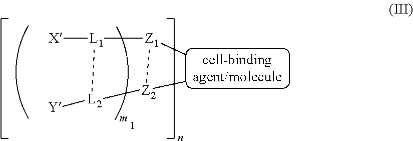

wherein:

m₁, n, “-----”, cell-binding agent/molecule, L₁, L₂, Z₁, and Z₂ are defined the same as in Formula (I);

X′ and Y′ are a function group that can independently react with a residue groups of a cytotoxic drug simultaneously or sequentially to form X and Y respectively, wherein X and Y are defined in Formula (I);

X′ and Y′ are preferably independently a disulfide substituent, maleimido, haloacetyl, alkoxyamine, azido, ketone, aldehyde, hydrazine, amino, hydroxyl, carboxylate, imidazole, thiol, or alkyne; or a N-hydroxysuccinimide ester, p-nitrophenyl ester, dinitrophenyl ester, pentafluorophenyl ester, pentachlorophenyl ester; tetrafluorophenyl ester; difluorophenyl ester; monofluorophenyl ester; or pentachlorophenyl ester, dichlorophenyl ester, tetrachlorophenyl ester, or 1-hydroxybenzotriazole ester; a triflate, mesylate, or tosylate; 2-ethyl-5-phenylisoxazolium-3′-sulfonate; a pyridyldisulfide, or nitropyridyldisulfide; a maleimide, haloacetate, acetylenedicarboxylic group, or carboxylic acid halogenate (fluoride, chloride, bromide, or iodide). Preferably X and Y have one of the following structures:

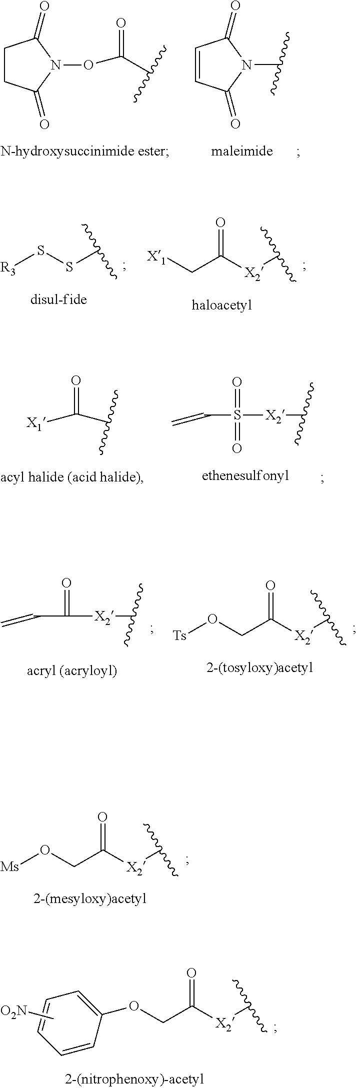

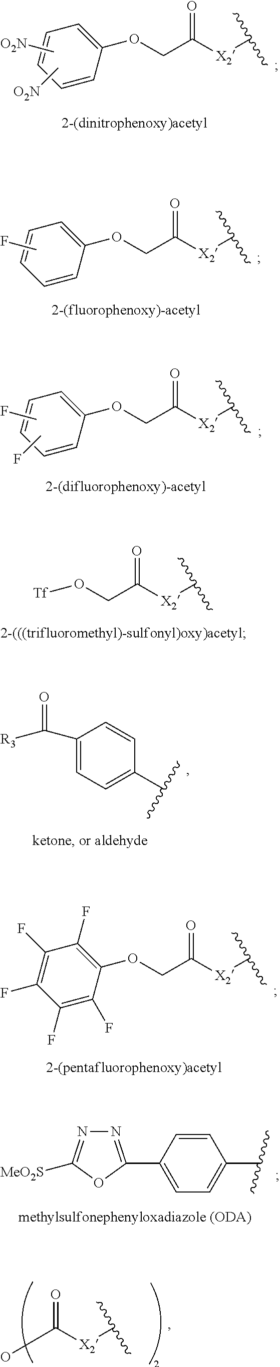

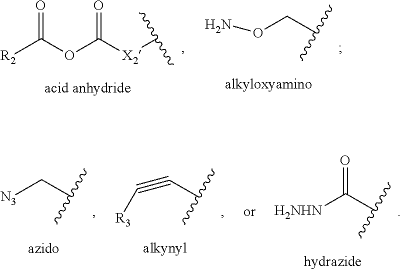

wherein X₁′ is F, Cl, Br, I or Lv₃; X₂′ is O, NH, N(R₁), or CH₂; R₃ and R₅ are H, R₁, aromatic, heteroaromatic, or aromatic group wherein one or several H atoms are replaced independently by —R₁, -halogen, —OR₁, —SR₁, —NR₁R₂, —NO₂, —S(O)R₁, —S(O)₂R₁, or —COOR₁; Lv₃ is a leaving group selected from methanesulfonyl (mesyl), toluenesulfonyl (tosyl), trifluoromethyl-sulfonyl (triflate), trifluoromethylsulfonate, nitrophenoxyl, N-succinimidyloxyl (NHS), phenoxyl; dinitrophenoxyl; pentafluorophenoxyl, tetrafluoro-phenoxyl, trifluorophenoxyl, difluorophenoxyl, monofluoro-phenoxyl, pentachlorophenoxyl, 1H-imidazole-1-yl, chlorophenoxyl, dichlorophenoxyl, trichlorophenoxyl, tetrachlorophenoxyl, N-(benzotriazol-yl)oxyl, 2-ethyl-5-phenylisoxazolium-yl, phenyloxadiazol-yl (ODA), oxadiazol-yl, or an intermediate molecule generated with a condensation reagent for Mitsunobu reactions, wherein R₁ and R₂ are defined above;

Preferably a bis-linker compound for preparation of the conjugate is further represented by Formula (III-a), (III-b), (III-c), (III-d), (III-e), (III-f), (III-g), (III-h), (III-i), (III-j), (III-k), (III-l), (III-m), (III-n), (III-o), (III-p), (III-r), (III-s), (III-t), (III-u), (III-v), and (III-w) below:

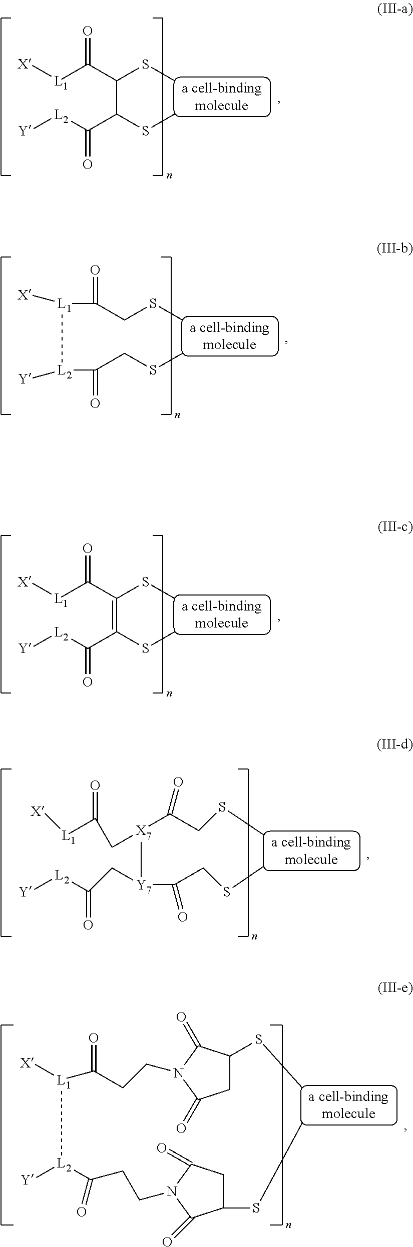

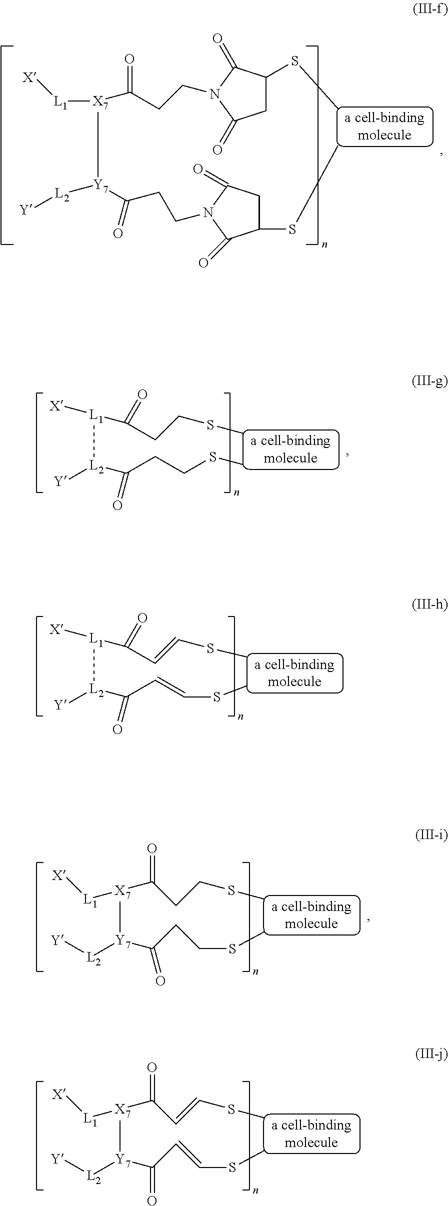

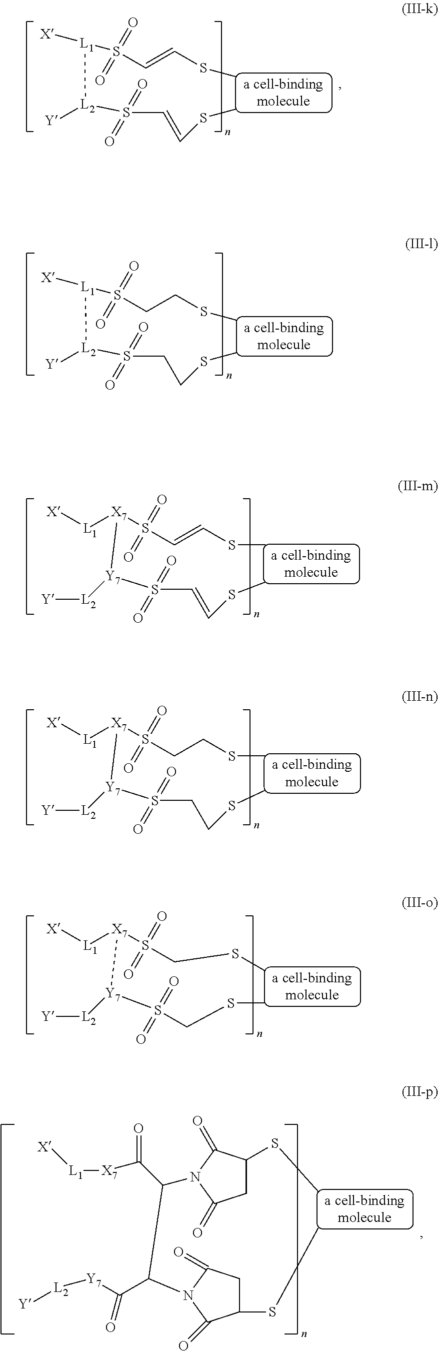

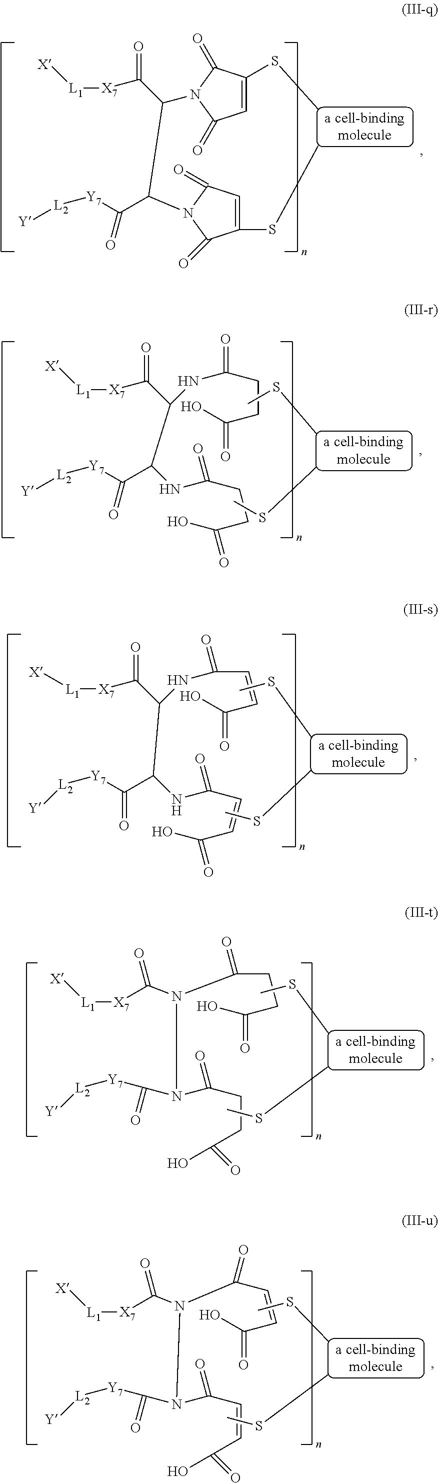

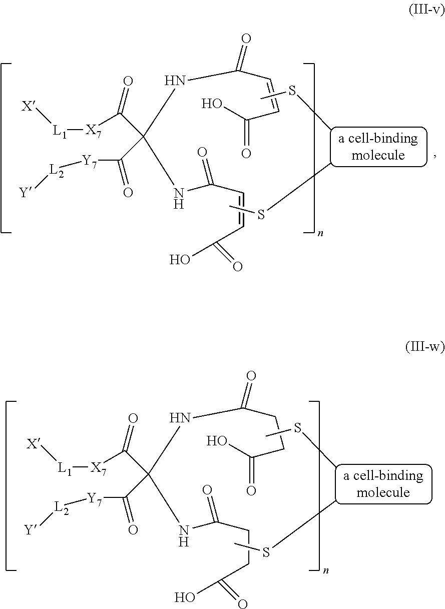

wherein X₇ and Y₇ are independently CH, CH₂, NH, O, S, NHNH, N(R₁), and N; a chemical bond in the middle of two atoms means it can link either adjoining two atoms; R₁, X′, Y′, n, L₁ and L₂ are the same described above.

In another aspect, this invention provides a readily-reactive bis-linker of Formula (IV) below, wherein a cytotoxic molecule and a cell-binding molecule can react it independently, or simultaneously, or sequentially to form Formula (I):

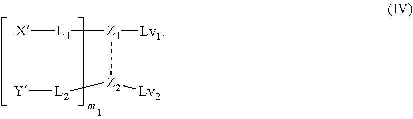

wherein “-----”, m₁, L₁, L₂, Z₁, and Z₂ are defined the same as in Formula (I); Lv₁ and Lv₂ are defined in Formula (II), and X′ and Y′ are defined in Formula (III);

Preferably the bis-linker for preparation of the conjugate is further represented by Formula (IV-a), (IV-b), (IV-c), (IV-d), (IV-e), (IV-f), (IV-g), (IV-h), (IV-i), (IV-j), (IV-k), (IV-m), (IV-n), (IV-o), (IV-p), (IV-q), (IV-r), and (IV-s):

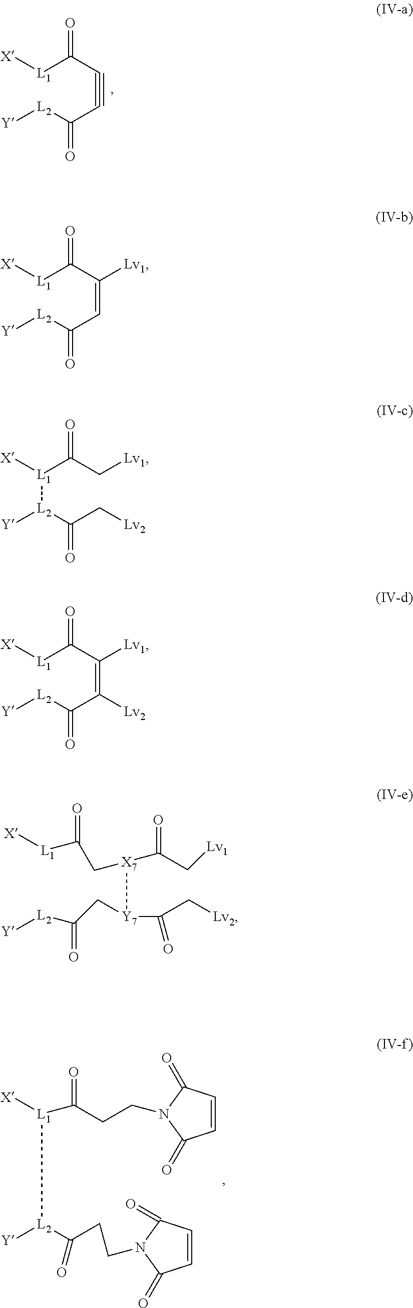

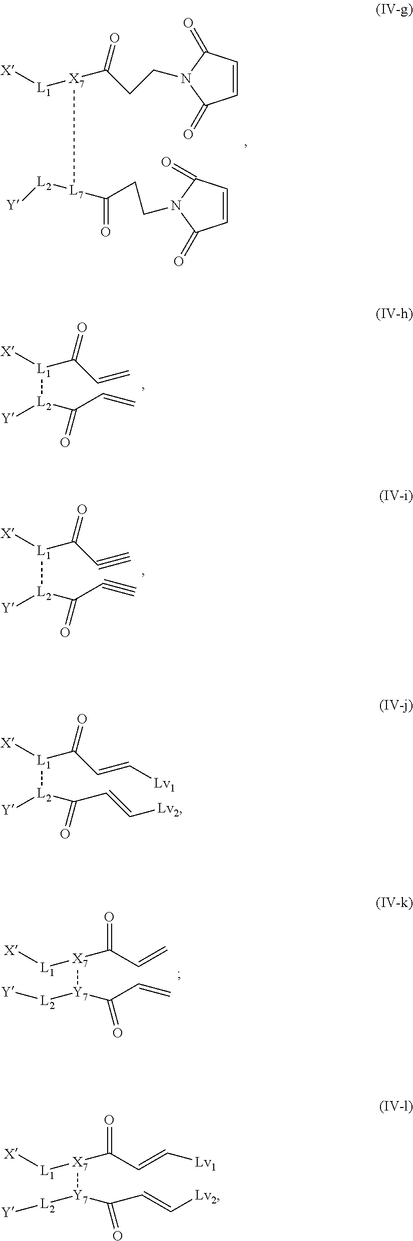

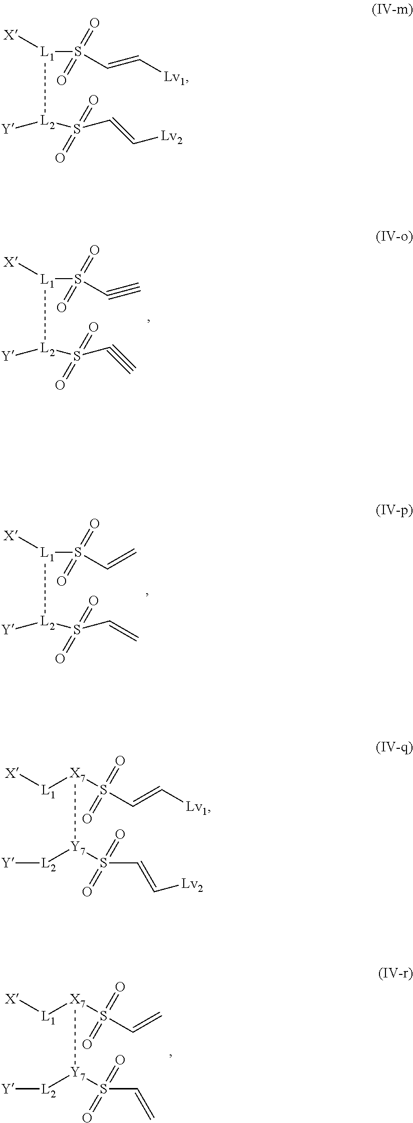

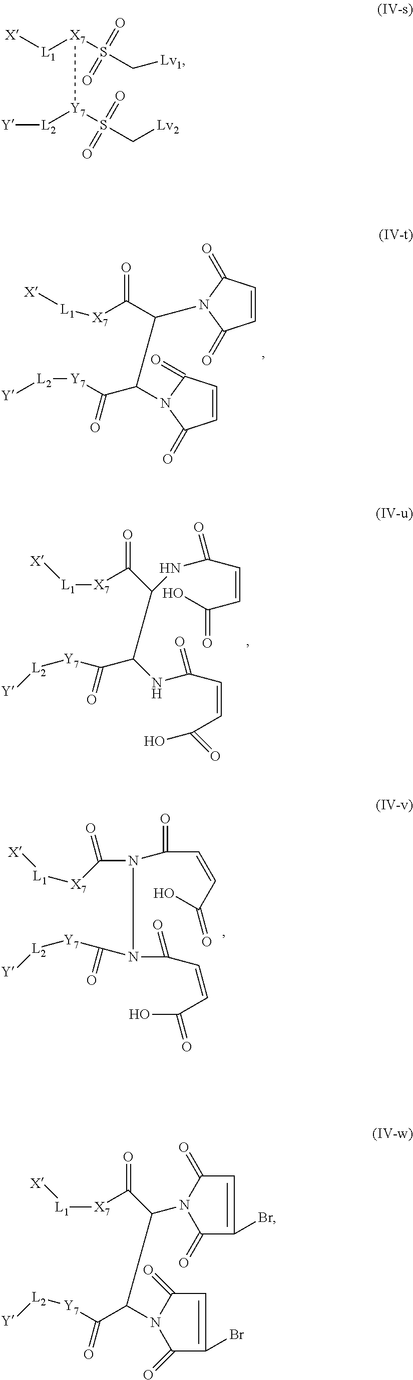

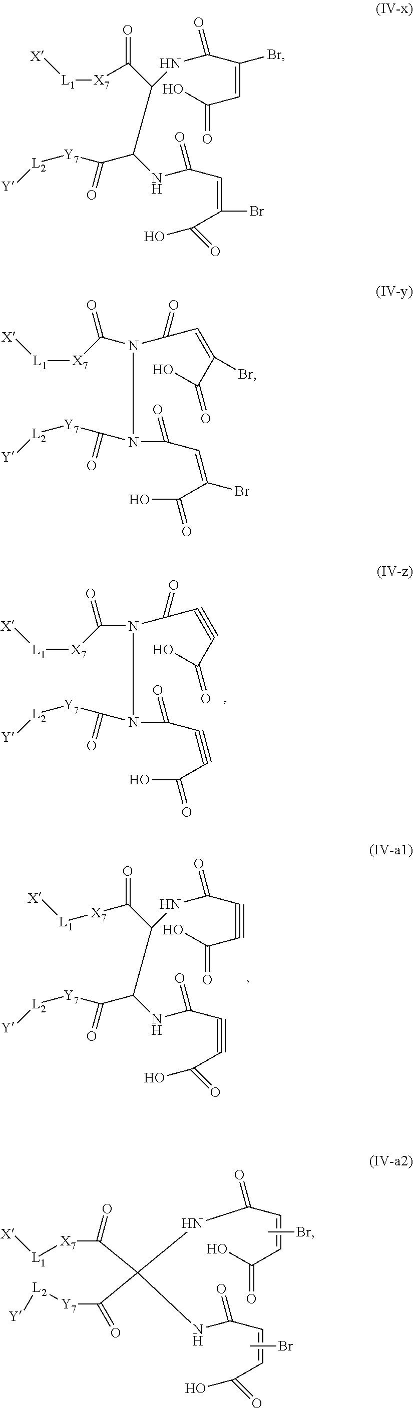

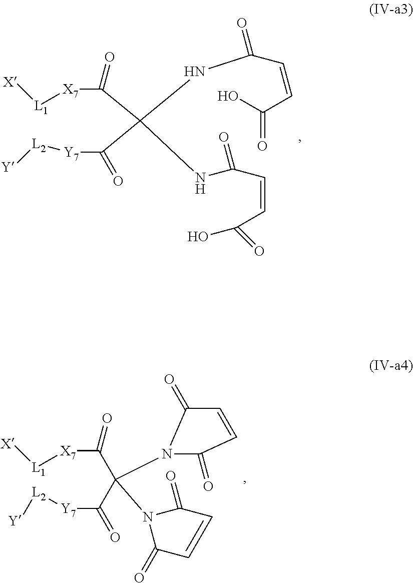

wherein X₇ and Y₇ are independently CH, CH₂, NH, O, S, NHNH, N(R₁), and N; a chemical bond in the middle of two atoms means it can link either adjoining two atoms; “-----”, R₁, X′, Y′, n, L₁ and L₂ are the same described above.

Examples of the functional groups, X′ or Y′, that enable reaction with the terminal of amine or hydroxyl group of a drug/cytotoxic agent, can be, but not limited to, N-hydroxysuccinimide esters, p-nitrophenyl esters, dinitrophenyl esters, pentafluorophenyl esters, carboxylic acid chlorides or carboxylic acid anhydride; With the terminal of thiol of a cytotoxic agent, can be, as but not limited to, pyridyldisulfides, nitropyridyldisulfides, maleimides, haloacetates, methylsulfonephenyloxadiazole (ODA), carboxylic acid chlorides and carboxylic acid anhydride; With the terminal of ketone or aldehyde, can be, but not limited to, amines, alkoxyamines, hydrazines, acyloxylamine, or hydrazide; With the terminal of azide, can be, as but not limited to, alkyne.

Preparation of Conjugates

The conjugates of Formula (I) can be prepared through the intermediate compounds of Formula (II), (III) or (IV) respectively. Some preparations of Formula (II) are structurally shown in the FIGS. 1˜40. To synthesize the conjugate of Formula (I), in general, two function groups on a drug or on a cell toxicity molecule first reacts sequentially or simultaneously to X′ group and Y′ group of the linker of Formula (IV) in a chemical solvent or in an aqueous media containing 0.1%-99.5% organic solvents or in 100% aqueous media to form a compound of Formula (II). Then the compound of Formula (II) can be optionally isolated first, or can immediately or simultaneously or sequentially react to two or more residues of a cell binding molecule, preferably a pair of free thiols generated through reduction of disulfide bonds of the cell-binding molecule at 0-60° C., pH 5˜9 aqueous media with or without addition of 0˜30% of water mixable (miscible) organic solvents, such as DMA, DMF, ethanol, methanol, acetone, acetonitrile, THF, isopropanol, dioxane, propylene glycol, or ethylene diol to form a conjugate compound of Formula (I).

Alternatively, the conjugates of the Formula (I) can also be obtained through the first reaction of the linkers of the Formula (IV) to two or more residues of a cell binding molecule, preferably a pair of free thiols generated through reduction of disulfide bonds of the cell-binding molecule at 0-60° C., pH 5˜9 aqueous media with or without addition of 0˜30% of water mixable (miscible) organic solvents, to form the modified cell-binding molecule of Formula (III). The pairs of thiols are preferred pairs of disulfide bonds reduced from the inter chain disulfide bonds of the cell-binding agent by a reduction agent which can selected from dithiothreitol (DTT), dithioerythritol (DTE), L-glutathione (GSH), tris (2-carboxyethyl) phosphine (TCEP), 2-mercaptoethylamine (β-MEA), or/and beta mercaptoethanol (β-ME, 2-ME) at pH4˜9 aqueous media with or without addition of 0˜30% of water mixable (miscible) organic solvents. The reactive groups of X′ and Y′ on Formula (III), which can be independently disulfide, thiol, thioester, maleimido, haloacetyl, azide, 1-yne, ketone, aldehyde, alkoxyamino, triflate, carbonylimidazole, tosylate, mesylate, 2-ethyl-5-phenylisoxazolium-3′-sulfonate, or carboxyl acid esters of nitrophenol, N-hydroxysuccinimide (NHS), phenol; dinitrophenol, pentafluorophenol, tetrafluorophenol, difluorophenol, monofluorophenol, pentachlorophenol, dichlorophenol, tetrachlorophenol, 1-hydroxybenzotriazole, anhydrides, or hydrazide groups, or other acid ester derivatives, can then react to two groups on a drug/cytotoxic agent, simultaneously or sequentially at 0-60° C., pH 4˜9.5 aqueous media with or without addition of 0˜30% of water mixable (miscible) organic solvents, to yield a conjugate of the Formula (I), after column purification or dialysis. The reactive groups of a drug/cytotoxic agent react to the modified cell-binding molecule of Formula (III) in different ways accordingly. For example, a linkage containing disulfide bonds in the cell-binding agent-drug conjugates of Formula (I) is achieved by a disulfide exchange between the disulfide bond in the modified cell-binding agent of Formula (III) and a drug having a free thiol group; A linkage containing thioether bonds in the cell-binding agent-drug conjugates of Formula (I) is achieved by reaction of the maleimido or haloacetyl or ethylsulfonyl modified cell-binding agent of Formula (III) and a drug having a free thiol group; A linkage containing a bond of an acid labile hydrazone in the conjugates can be achieved by reaction of a carbonyl group of the drug or compound of Formula (III) with the hydrazide moiety on compound of Formula (III) or the drug accordingly, by methods known in the art (see, for example, P. Hamann et al., Cancer Res. 53, 3336-34, 1993; B. Laguzza et al., J. Med. Chem., 32; 548-55, 1959; P. Trail et al., Cancer Res., 57; 100-5, 1997); A linkage containing a bond of triazole in the conjugates can be achieved by reaction of a 1-yne group of the drug or compound of Formula (III) with the azido moiety on the other counterpart accordingly, through the click chemistry (Huisgen cycloaddition) (Lutz, J-F. et al, 2008, Adv. Drug Del. Rev. 60, 958-70; Sletten, E. M. et al 2011, AccChem. Research 44, 666-76). A linkage containing a bond of oxime in the cell-binding agent-drug conjugates linked via oxime is achieved by reaction of a group of a ketone or aldehyde on the modified cell-binding agent of Formula (III) or a drug with a group of oxyamine on a drug or the modified cell-binding agent of Formula (III) respectively. A thiol-containing drug can react with the modified cell-binding molecule linker of Formula (III) bearing a maleimido, or a haloacetyl, or an ethylsulfonyl substituent at pH 5.5˜9.0 in aqueous buffer to give a thioether linkage in cell-binding molecule-drug conjugate of Formula (I). A thiol-containing drug can undergo disulfide exchange with a modified linker of Formula (III) bearing a pyridyldithio moiety to give a conjugate having a disulfide bond linkage. A drug bearing a hydroxyl group or a thiol group can be reacted with a modified bridge linker of Formula (III) bearing a halogen, particularly the alpha halide of carboxylates, in the presence of a mild base, e.g. pH 8.0˜9.5, to give a modified drug bearing an ether or thiol ether linkage. A hydroxyl group on a drug can be condensed with a cross linker of Formula (IV) bearing a carboxyl group, in the presence of a dehydrating agent, such as EDC or DCC, to give ester linkage, then the subject drug modified bridge linker of Formula (III) undergoes the conjugation with a cell-binding molecule. A drug containing an amino group can condensate with a group of carboxyl ester of NHS, imidazole, nitrophenol; N-hydroxysuccinimide (NHS); phenol; dinitrophenol; pentafluorophenol; tetrafluorophenol; difluorophenol; monofluorophenol; pentachlorophenol; triflate; imidazole; dichlorophenol; tetrachlorophenol; 1-hydroxyben-zotriazole; tosylate; mesylate; 2-ethyl-5-phenylisoxazolium-3′-sulfonate on the cell-binding molecule-linker of Formula (III) to give a conjugate via amide bond linkage.

The synthetic conjugate may be purified by standard biochemical means, such as gel filtration on a Sephadex G25 or Sephacryl S300 column, adsorption chromatography, and ion exchange or by dialysis. In some cases, a small molecule as a cell-binding agent (e.g. folic acid, melanocyte stimulating hormone, EGF etc.) conjugated with a small molecular drugs can be purified by chromatography such as by HPLC, medium pressure column chromatography or ion exchange chromatography.

In order to achieve a higher yield of conjugation reaction of the cytotoxic molecule-bis linker complex of the Formula (II) with a pair of free thiols on the cell-binding molecule, preferably on an antibody, a small percentage of water miscible organic solvents, or phase transfer agents, may be required to add to the reaction mixture. To cross-linking reagent (linker) of Formula (II) can be first dissolved in a polar organic solvent that is miscible with water, for example in different alcohols, such as methanol, ethanol, and propanol, acetone, acetonitrile, tetrahydrofuran (THF), 1,4-dioxane, dimethyl formamide (DMF), dimethyl acetamide (DMA), or dimethylsulfoxide (DMSO) at a high concentration, for example 1-500 mM. Meanwhile, the cell-binding molecule, such as antibody dissolved in an aqueous buffer pH 4˜9.5, preferably pH 6˜8.5, at 1˜50 mg/ml concentration was treated with 0.5˜20 equivalent of TCEP or DTT for 20 min to 48 hour. After the reduction, DTT can be removed by SEC chromatographic purification. TCEP can be optionally removed by SEC chromatography too, or staying in the reaction mixture for the next step reaction without further purification. Furthermore, the reduction of antibodies or the other cell-binding agents with TCEP can be performed along with existing a drug-linker molecule of Formula (II), for which the cross-linking conjugation of the cell-binding molecules can be achieved simultaneously along with the TCEP reduction.

The aqueous solutions for the modification of cell-binding agents are buffered between pH 4 and 9, preferably between 6.0 and 7.5 and can contain any non-nucleophilic buffer salts useful for these pH ranges. Typical buffers include phosphate, acetate, triethanolamine HCl, HEPES, and MOPS buffers, which can contain additional components, such as cyclodextrins, Hydroxypropyl-β-cyclodextrin, polyethylene glycols, sucrose and salts, for examples, NaCl and KCl. After the addition of the drug-linker of Formula (II) into the solution containing the reduced cell-binding molecules, the reaction mixture is incubated at a temperature of from 4 OC to 45° C., preferably at 15° C.—ambient temperature. The progress of the reaction can be monitored by measuring the decrease in the absorption at a certain UV wavelength, such as at 254 nm, or increase in the absorption at a certain UV wavelength, such as 280 nm, or the other appropriate wavelength. After the reaction is complete, isolation of the modified cell-binding agent can be performed in a routine way, using for example a gel filtration chromatography, an ion exchange chromatography, an adsorptive chromatography or column chromatography over silica gel or alumina, crystallization, preparatory thin layer chromatography, ion exchange chromatography, or HPLC.

The extent of modification can be assessed by measuring the absorbance of the nitropyridine thione, dinitropyridine dithione, pyridine thione, carboxylamidopyridine dithione and dicarboxyl-amidopyridine dithione group released via UV spectra. For the conjugation without a chromophore group, the modification or conjugation reaction can be monitored by LC-MS, preferably by UPLC-QTOF mass spectrometry, or Capilary electrophoresis-mass spectrometry (CE-MS). The bridge cross-linkers described herein have diverse functional groups that can react with any drugs, preferably cytotoxic agents that possess a suitable substituent. For examples, the modified cell-binding molecules bearing an amino or hydroxyl substituent can react with drugs bearing an N-hydroxysuccinimide (NHS) ester, the modified cell-binding molecules bearing a thiol substituent can react with drugs bearing a maleimido or haloacetyl group. Additionally, the modified cell-binding molecules bearing a carbonyl (ketone or aldehyde) substituent can react with drugs bearing a hydrazide or an alkoxyamine. One skilled in the art can readily determine which linker to use based on the known reactivity of the available functional group on the linkers.

Cell-Binding Agents

The cell-binding molecule, Cb, that comprises the conjugates and the modified cell-binding agents of the present invention may be of any kind presently known, or that become known, molecule that binds to, complexes with, or reacts with a moiety of a cell population sought to be therapeutically or otherwise biologically modified.

The cell binding agents include, but are not limited to, large molecular weight proteins such as, for example, antibody, an antibody-like protein, full-length antibodies (polyclonal antibodies, monoclonal antibodies, dimers, multimers, multispecific antibodies (e.g., a bispecific antibody, trispecific antibody, or tetraspecific antibody); single chain antibodies; fragments of antibodies such as Fab, Fab′, F(ab′)₂, Fᵥ, [Parham, J. Immunol. 131, 2895-902 (1983)], fragments produced by a Fab expression library, anti-idiotypic (anti-Id) antibodies, CDR's, diabody, triabody, tetrabody, miniantibody, a probody, a probody fragment, small immune proteins (SIP), and epitope-binding fragments of any of the above which immuno-specifically bind to cancer cell antigens, viral antigens, microbial antigens or a protein generated by the immune system that is capable of recognizing, binding to a specific antigen or exhibiting the desired biological activity (Miller et al (2003) J. of Immunology 170: 4854-61); interferons (such as type I, II, III); peptides; lymphokines such as IL-2, IL-3, IL-4, IL-5, IL-6, IL-10, GM-CSF, interferon-gamma (IFN-γ); hormones such as insulin, TRH (thyrotropin releasing hormones), MSH (melanocyte-stimulating hormone), steroid hormones, such as androgens and estrogens, melanocyte-stimulating hormone (MSH); growth factors and colony-stimulating factors such as epidermal growth factors (EGF), granulocyte-macrophage colony-stimulating factor (GM-CSF), transforming growth factors (TGF), such as TGFα, TGFβ, insulin and insulin like growth factors (IGF-I, IGF-II) G-CSF, M-CSF and GM-CSF [Burgess, Immunology Today, 5, 155-8 (1984)]; vaccinia growth factors (VGF); fibroblast growth factors (FGFs); smaller molecular weight proteins, poly-peptide, peptides and peptide hormones, such as bombesin, gastrin, gastrin-releasing peptide; platelet-derived growth factors; interleukin and cytokines, such as interleukin-2 (IL-2), interleukin-6 (IL-6), leukemia inhibitory factors, granulocyte-macrophage colony-stimulating factor (GM-CSF); vitamins, such as folate; apoproteins and glycoproteins, such as transferrin [O'Keefe et al, 260 J. Biol. Chem. 932-7 (1985)]; sugar-binding proteins or lipoproteins, such as lectins; cell nutrient-transport molecules; and small molecular inhibitors, such as prostate-specific membrane antigen (PSMA) inhibitors and small molecular tyrosine kinase inhibitors (TKI), non-peptides or any other cell binding molecule or substance, such as bioactive polymers (Dhar, et al, Proc. Natl. Acad. Sci. 2008, 105, 17356-61); bioactive dendrimers (Lee, et al, Nat. Biotechnol. 2005, 23, 1517-26; Almutairi, et al; Proc. Natl. Acad. Sci. 2009, 106, 685-90); nanoparticles (Liong, et al, ACS Nano, 2008, 2, 1309-12; Medarova, et al, Nat. Med. 2007, 13, 372-7; Javier, et al, Bioconjugate Chem. 2008, 19, 1309-12); liposomes (Medinai, et al, Curr. Phar. Des. 2004, 10, 2981-9); viral capsides (Flenniken, et al, Viruses Nanotechnol. 2009, 327, 71-93).

In general, a monoclonal antibody is preferred as a cell-surface binding agent if an appropriate one is available. And the antibody may be murine, human, humanized, chimeric, or derived from other species.

Production of antibodies used in the present invention involves in vivo or in vitro procedures or combinations thereof. Methods for producing polyclonal anti-receptor peptide antibodies are well-known in the art, such as in U.S. Pat. No. 4,493,795 (to Nestor et al). A monoclonal antibody is typically made by fusing myeloma cells with the spleen cells from a mouse that has been immunized with the desired antigen (Köhler, G.; Milstein, C. (1975). Nature 256: 495-7). The detailed procedures are described in “Antibodies—A Laboratory Manual”, Harlow and Lane, eds., Cold Spring Harbor Laboratory Press, New York (1988), which is incorporated herein by reference. Particularly monoclonal antibodies are produced by immunizing mice, rats, hamsters or any other mammal with the antigen of interest such as the intact target cell, antigens isolated from the target cell, whole virus, attenuated whole virus, and viral proteins. Splenocytes are typically fused with myeloma cells using polyethylene glycol (PEG) 6000. Fused hybrids are selected by their sensitivity to HAT (hypoxanthine-aminopterin-thymine). Hybridomas producing a monoclonal antibody useful in practicing this invention are identified by their ability to immunoreact specified receptors or inhibit receptor activity on target cells.

A monoclonal antibody used in the present invention can be produced by initiating a monoclonal hybridoma culture comprising a nutrient medium containing a hybridoma that secretes antibody molecules of the appropriate antigen specificity. The culture is maintained under conditions and for a time period sufficient for the hybridoma to secrete the antibody molecules into the medium. The antibody-containing medium is then collected. The antibody molecules can then be further isolated by well-known techniques, such as using protein-A affinity chromatography; anion, cation, hydrophobic, or size exclusive chromatographies (particularly by affinity for the specific antigen after protein A, and sizing column chromatography); centrifugation, differential solubility, or by any other standard technique for the purification of proteins.

Media useful for the preparation of these compositions are both well-known in the art and commercially available and include synthetic culture media. An exemplary synthetic medium is Dulbecco's minimal essential medium (DMEM; Dulbecco et al., Virol. 8, 396 (1959)) supplemented with 4.5 gm/l glucose, 0˜20 mM glutamine, 0˜20% fetal calf serum, several ppm amount of heavy metals, such as Cu, Mn, Fe, or Zn, etc., or/and the other heavy metals added in their salt forms, and with an anti-foaming agent, such as polyoxyethylene-polyoxypropylene block copolymer.

In addition, antibody-producing cell lines can also be created by techniques other than fusion, such as direct transformation of B lymphocytes with oncogenic DNA, or transfection with an oncovirus, such as Epstein-Barr virus (EBV, also called human herpesvirus 4 (HHV-4)) or Kaposi's sarcoma-associated herpesvirus (KSHV). See, U.S. Pat. Nos. 4,341,761; 4,399,121; 4,427,783; 4,444,887; 4,451,570; 4,466,917; 4,472,500; 4,491,632; 4,493,890. A monoclonal antibody may also be produced via an anti-receptor peptide or peptides containing the carboxyl terminal as described well-known in the art. See Niman et al., Proc. Natl. Acad. Sci. USA, 80: 4949-53 (1983); Geysen et al., Proc. Natl. Acad. Sci. USA, 82: 178-82 (1985); Lei et al. Biochemistry 34(20): 6675-88, (1995). Typically, the anti-receptor peptide or a peptide analog is used either alone or conjugated to an immunogenic carrier, as the immunogen for producing anti-receptor peptide monoclonal antibodies.

There are also a number of other well-known techniques for making monoclonal antibodies as binding molecules in this invention. Particularly useful are methods of making fully human antibodies. One method is phage display technology which can be used to select a range of human antibodies binding specifically to the antigen using methods of affinity enrichment. Phage display has been thoroughly described in the literature and the construction and screening of phage display libraries are well known in the art, see, e.g., Dente et al, Gene. 148(1):7-13 (1994); Little et al, Biotechnol Adv. 12(3): 539-55 (1994); Clackson et al., Nature 352: 264-8 (1991); Huse et al., Science 246: 1275-81 (1989).

Monoclonal antibodies derived by hybridoma technique from another species than human, such as mouse, can be humanized to avoid human anti-mouse antibodies when infused into humans. Among the more common methods of humanization of antibodies are complementarity-determining region grafting and resurfacing. These methods have been extensively described, see e.g. U.S. Pat. Nos. 5,859,205 and 6,797,492; Liu et al, Immunol Rev. 222: 9-27 (2008); Almagro et al, Front Biosci. 13: 1619-33 (2008); Lazar et al, Mol Immunol. 44(8): 1986-98 (2007); Li et al, Proc. Natl. Acad. Sci. USA. 103(10): 3557-62 (2006) each incorporated herein by reference. Fully human antibodies can also be prepared by immunizing transgenic mice, rabbits, monkeys, or other mammals, carrying large portions of the human immunoglobulin heavy and light chains, with an immunogen. Examples of such mice are: the Xenomouse. (Abgenix/Amgen), the HuMAb-Mouse (Medarex/BMS), the VelociMouse (Regeneron), see also U.S. Pat. Nos. 6,596,541, 6,207,418, 6,150,584, 6,111,166, 6,075,181, 5,922,545, 5,661,016, 5,545,806, 5,436,149 and 5,569,825. In human therapy, murine variable regions and human constant regions can also be fused to construct called “chimeric antibodies” that are considerably less immunogenic in man than murine mAbs (Kipriyanov et al, Mol Biotechnol. 26: 39-60 (2004); Houdebine, Curr Opin Biotechnol. 13: 625-9 (2002) each incorporated herein by reference). In addition, site-directed mutagenesis in the variable region of an antibody can result in an antibody with higher affinity and specificity for its antigen (Brannigan et al, Nat Rev Mol Cell Biol. 3: 964-70, (2002)); Adams et al, J Immunol Methods. 231: 249-60 (1999)) and exchanging constant regions of a mAb can improve its ability to mediate effector functions of binding and cytotoxicity.

Antibodies immunospecific for a malignant cell antigen can also be obtained commercially or produced by any method known to one of skill in the art such as, e.g., chemical synthesis or recombinant expression techniques. The nucleotide sequence encoding antibodies immune-specific for a malignant cell antigen can be obtained commercially, e.g., from the GenBank database or a database like it, the literature publications, or by routine cloning and sequencing.

Apart from an antibody, a peptide or protein that bind/block/target or in some other way interact with the epitopes or corresponding receptors on a targeted cell can be used as a binding molecule. These peptides or proteins could be any random peptide or proteins that have an affinity for the epitopes or corresponding receptors and they don't necessarily have to be of the immune-globulin family. These peptides can be isolated by similar techniques as for phage display antibodies (Szardenings, J Recept Signal Transduct Res. 2003, 23(4): 307-49). The use of peptides from such random peptide libraries can be similar to antibodies and antibody fragments. The binding molecules of peptides or proteins may be conjugated on or linked to a large molecules or materials, such as, but is not limited, an albumin, a polymer, a liposome, a nano particle, a dendrimer, as long as such attachment permits the peptide or protein to retain its antigen binding specificity.

Examples of antibodies used for conjugation of drugs via the linkers of this prevention for treating cancer, autoimmune disease, and/or infectious disease include, but are not limited to, 3F8 (anti-GD2), Abagovomab (anti CA-125), Abciximab (anti CD41 (integrin alpha-IIb), Adalimumab (anti-TNF-α), Adecatumumab (anti-EpCAM, CD326), Afelimomab (anti-TNF-α); Afutuzumab (anti-CD20), Alacizumab pegol (anti-VEGFR2), ALD518 (anti-IL-6), Alemtuzumab (Campath, MabCampath, anti-CD52), Altumomab (anti-CEA), Anatumomab (anti-TAG-72), Anrukinzumab (IMA-638, anti-IL-13), Apolizumab (anti-HLA-DR), Arcitumomab (anti-CEA), Aselizumab (anti-L-selectin (CD62L), Atlizumab (tocilizumab, Actemra, RoActemra, anti-IL-6 receptor), Atorolimumab (anti-Rhesus factor), Bapineuzumab (anti-beta amyloid), Basiliximab (Simulect, antiCD25 (a chain of IL-2 receptor), Bavituximab (anti-phosphatidylserine), Bectumomab (LymphoScan, anti-CD22), Belimumab (Benlysta, LymphoStat-B, anti-BAFF), Benralizumab (anti-CD125), Bertilimumab (anti-CCL11 (eotaxin-1)), Besilesomab (Scintimun, anti-CEA-related antigen), Bevacizumab (Avastin, anti-VEGF-A), Biciromab (FibriScint, anti-fibrin II beta chain), Bivatuzumab (anti-CD44 v6), Blinatumomab (BiTE, anti-CD19), Brentuximab (cAC10, anti-CD30 TNFRSF8), Briakinumab (anti-IL-12, IL-23) Canakinumab (Ilaris, anti-IL-1), Cantuzumab (C242, anti-CanAg), Capromab, Catumaxomab (Removab, anti-EpCAM, anti-CD3), CC49 (anti-TAG-72), Cedelizumab (anti-CD4), Certolizumab pegol (Cimzia anti-TNF-α), Cetuximab (Erbitux, IMC-C225, anti-EGFR), Citatuzumab bogatox (anti-EpCAM), Cixutumumab (anti-IGF-1), Clenoliximab (anti-CD4), Clivatuzumab (anti-MUC1), Conatumumab (anti-TRAIL-R2), CR6261 (anti-Influenza A hemagglutinin), Dacetuzumab (anti-CD40), Daclizumab (Zenapax, anti-CD25 (α chain of IL-2 receptor)), Daratumumab (anti-CD38 (cyclic ADP ribose hydrolase), Denosumab (Prolia, anti-RANKL), Detumomab (anti-B-lymphoma cell), Dorlimomab, Dorlixizumab, Ecromeximab (anti-GD3 ganglioside), Eculizumab (Soliris, anti-C5), Edobacomab (anti-endotoxin), Edrecolomab (Panorex, MAb 17-1A, anti-EpCAM), Efalizumab (Raptiva, anti-LFA-1 (CD11a), Efungumab (Mycograb, anti-Hsp90), Elotuzumab (anti-SLAMF7), Elsilimomab (anti-IL-6), Enlimomab pegol (anti-ICAM-1 (CD54)), Epitumomab (anti-episialin), Epratuzumab (anti-CD22), Erlizumab (anti-ITGB2 (CD18)), Ertumaxomab (Rexomun, anti-HER2/neu, CD3), Etaracizumab (Abegrin, anti-integrin αᵥβ₃), Exbivirumab (anti-hepatitis B surface antigen), Fanolesomab (NeutroSpec, anti-CD15), Faralimomab (anti-interferon receptor), Farletuzumab (anti-folate receptor 1), Felvizumab (anti-respiratory syncytial virus), Fezakinumab (anti-IL-22), Figitumumab (anti-IGF-1 receptor), Fontolizumab (anti-IFN-γ), Foravirumab (anti-rabies virus glycoprotein), Fresolimumab (anti-TGF-β), Galiximab (anti-CD80), Gantenerumab (anti-beta amyloid), Gavilimomab (anti-CD147 (basigin)), Gemtuzumab (anti-CD33), Girentuximab (anti-carbonic anhydrase 9), Glembatumumab (CR011, anti-GPNMB), Golimumab (Simponi, anti-TNF-α), Gomiliximab (anti-CD23 (IgE receptor)), Ibalizumab (anti-CD4), Ibritumomab (anti-CD20), Igovomab (Indimacis-125, anti-CA-125), Imciromab (Myoscint, anti-cardiac myosin), Infliximab (Remicade, anti-TNF-α), Intetumumab (anti-CD51), Inolimomab (anti-CD25 (a chain of IL-2 receptor)), Inotuzumab (anti-CD22), Ipilimumab (anti-CD152), Iratumumab (anti-CD30 (TNFRSF8)), Keliximab (anti-CD4), Labetuzumab (CEA-Cide, anti-CEA), Lebrikizumab (anti-IL-13), Lemalesomab (anti-NCA-90 (granulocyte antigen)), Lerdelimumab (anti-TGF beta 2), Lexatumumab (anti-TRAIL-R2), Libivirumab (anti-hepatitis B surface antigen), Lintuzumab (anti-CD33), Lucatumumab (anti-CD40), Lumiliximab (anti-CD23 (IgE receptor), Mapatumumab (anti-TRAIL-R1), Maslimomab (anti-T-cell receptor), Matuzumab (anti-EGFR), Mepolizumab (Bosatria, anti-IL-5), Metelimumab (anti-TGF beta 1), Milatuzumab (anti-CD74), Minretumomab (anti-TAG-72), Mitumomab (BEC-2, anti-GD3 ganglioside), Morolimumab (anti-Rhesus factor), Motavizumab (Numax, anti-respiratory syncytial virus), Muromonab-CD3 (Orthoclone OKT3, anti-CD3), Nacolomab (anti-C242), Naptumomab (anti-5T4), Natalizumab (Tysabri, anti-integrin α₄), Nebacumab (anti-endotoxin), Necitumumab (anti-EGFR), Nerelimomab (anti-TNF-α), Nimotuzumab (Theracim, Theraloc, anti-EGFR), Nofetumomab, Ocrelizumab (anti-CD20), Odulimomab (Afolimomab, anti-LFA-1 (CD11a)), Ofatumumab (Arzerra, anti-CD20), Olaratumab (anti-PDGF-R α), Omalizumab (Xolair, anti-IgE Fc region), Oportuzumab (anti-EpCAM), Oregovomab (OvaRex, anti-CA-125), Otelixizumab (anti-CD3), Pagibaximab (anti-lipoteichoic acid), Palivizumab (Synagis, Abbosynagis, anti-respiratory syncytial virus), Panitumumab (Vectibix, ABX-EGF, anti-EGFR), Panobacumab (anti-Pseudomonas aeruginosa), Pascolizumab (anti-IL-4), Pemtumomab (Theragyn, anti-MUC1), Pertuzumab (Omnitarg, 2C4, anti-HER2/neu), Pexelizumab (anti-C5), Pintumomab (anti-adenocarcinoma antigen), Priliximab (anti-CD4), Pritumumab (anti-vimentin), PRO 140 (anti-CCR5), Racotumomab (1E10, anti-(N-glycolylneuraminic acid (NeuGc, NGNA)-gangliosides GM3)), Rafivirumab (anti-rabies virus glycoprotein), Ramucirumab (anti-VEGFR2), Ranibizumab (Lucentis, anti-VEGF-A), Raxibacumab (anti-anthrax toxin, protective antigen), Regavirumab (anti-cytomegalovirus glycoprotein B), Reslizumab (anti-IL-5), Rilotumumab (anti-HGF), Rituximab (MabThera, Rituxanmab, anti-CD20), Robatumumab (anti-IGF-1 receptor), Rontalizumab (anti-IFN-α), Rovelizumab (LeukArrest, anti-CD11, CD18), Ruplizumab (Antova, anti-CD154 (CD40L)), Satumomab (anti-TAG-72), Sevirumab (anti-cytomegalovirus), Sibrotuzumab (anti-FAP), Sifalimumab (anti-IFN-α), Siltuximab (anti-IL-6), Siplizumab (anti-CD2), (Smart) MI95 (anti-CD33), Solanezumab (anti-beta amyloid), Sonepcizumab (anti-sphingosine-1-phosphate), Sontuzumab (anti-episialin), Stamulumab (anti-myostatin), Sulesomab (LeukoScan, (anti-NCA-90 (granulocyte antigen), Tacatuzumab (anti-alpha-fetoprotein), Tadocizumab (anti-integrin α_{IIb}β₃), Talizumab (anti-IgE), Tanezumab (anti-NGF), Taplitumomab (anti-CD19), Tefibazumab (Aurexis, (anti-clumping factor A), Telimomab, Tenatumomab (anti-tenascin C), Teneliximab (anti-CD40), Teplizumab (anti-CD3), TGN1412 (anti-CD28), Ticilimumab (Tremelimumab, (anti-CTLA-4), Tigatuzumab (anti-TRAIL-R2), TNX-650 (anti-IL-13), Tocilizumab (Atlizumab, Actemra, RoActemra, (anti-IL-6 receptor), Toralizumab (anti-CD154 (CD40L)), Tositumomab (anti-CD20), Trastuzumab (Herceptin, (anti-HER2/neu), Tremelimumab (anti-CTLA-4), Tucotuzumab celmoleukin (anti-EpCAM), Tuvirumab (anti-hepatitis B virus), Urtoxazumab (anti-Escherichia coli), Ustekinumab (Stelara, anti-IL-12, IL-23), Vapaliximab (anti-AOC3 (VAP-1)), Vedolizumab, (anti-integrin α₄β₇), Veltuzumab (anti-CD20), Vepalimomab (anti-AOC3 (VAP-1), Visilizumab (Nuvion, anti-CD3), Vitaxin (anti-vascular integrin avb3), Volociximab (anti-integrin α₅β₁), Votumumab (HumaSPECT, anti-tumor antigen CTAA16.88), Zalutumumab (HuMax-EGFr, (anti-EGFR), Zanolimumab (HuMax-CD4, anti-CD4), Ziralimumab (anti-CD147 (basigin)), Zolimomab (anti-CD5), Etanercept (Enbrel®), Alefacept (Amevive®), Abatacept (Orencia®), Rilonacept (Arcalyst), 14F7 [anti-IRP-2 (Iron Regulatory Protein 2)], 14G2a (anti-GD2 ganglioside, from Nat. Cancer Inst. for melanoma and solid tumors), J591 (anti-PSMA, Weill Cornell Medical School for prostate cancers), 225.28S [anti-HMW-MAA (High molecular weight-melanoma-associated antigen), Sorin Radiofarmaci S.R.L. (Milan, Italy) for melanoma], COL-1 (anti-CEACAM3, CGM1, from Nat. Cancer Inst. USA for colorectal and gastric cancers), CYT-356 (Oncoltad®, for prostate cancers), HNK20 (OraVax Inc. for respiratory syncytial virus), ImmuRAIT (from Immunomedics for NHL), Lym-1 (anti-HLA-DR10, Peregrine Pharm. for Cancers), MAK-195F [anti-TNF (tumor necrosis factor; TNFA, TNF-alpha; TNFSF2), from Abbott/Knoll for Sepsis toxic shock], MEDI-500 [T10B9, anti-CD3, TRαβ (T cell receptor alpha/beta), complex, from MedImmune Inc for Graft-versus-host disease], RING SCAN [anti-TAG 72 (tumour associated glycoprotein 72), from Neoprobe Corp. for Breast, Colon and Rectal cancers], Avicidin (anti-EPCAM (epithelial cell adhesion molecule), anti-TACSTD1 (Tumor-associated calcium signal transducer 1), anti-GA733-2 (gastrointestinal tumor-associated protein 2), anti-EGP-2 (epithelial glycoprotein 2); anti-KSA; KS1/4 antigen; M4S; tumor antigen 17-1A; CD326, from NeoRx Corp. for Colon, Ovarian, Prostate cancers and NHL]; LymphoCide (Immunomedics, NJ), Smart ID10 (Protein Design Labs), Oncolym (Techniclone Inc, CA), Allomune (BioTransplant, CA), anti-VEGF (Genentech, CA); CEAcide (Immunomedics, NJ), IMC-1C11 (ImClone, NJ) and Cetuximab (ImClone, NJ).

Other antibodies as cell binding molecules/ligands include, but are not limited to, are antibodies against the following antigens: Aminopeptidase N (CD13), Annexin A1, B7-H3 (CD276, various cancers), CA125 (ovarian), CA15-3 (carcinomas), CA19-9 (carcinomas), L6 (carcinomas), Lewis Y (carcinomas), Lewis X (carcinomas), alpha fetoprotein (carcinomas), CA242 (colorectal), placental alkaline phosphatase (carcinomas), prostate specific antigen (prostate), prostatic acid phosphatase (prostate), epidermal growth factor (carcinomas), CD2 (Hodgkin's disease, NHL lymphoma, multiple myeloma), CD3 epsilon (T cell lymphoma, lung, breast, gastric, ovarian cancers, autoimmune diseases, malignant ascites), CD19 (B cell malignancies), CD20 (non-Hodgkin's lymphoma), CD22 (leukemia, lymphoma, multiple myeloma, SLE), CD30 (Hodgkin's lymphoma), CD33 (leukemia, autoimmune diseases), CD38 (multiple myeloma), CD40 (lymphoma, multiple myeloma, leukemia (CLL)), CD51 (Metastatic melanoma, sarcoma), CD52 (leukemia), CD56 (small cell lung cancers, ovarian cancer, Merkel cell carcinoma, and the liquid tumor, multiple myeloma), CD66e (cancers), CD70 (metastatic renal cell carcinoma and non-Hodgkin lymphoma), CD74 (multiple myeloma), CD80 (lymphoma), CD98 (cancers), mucin (carcinomas), CD221 (solid tumors), CD227 (breast, ovarian cancers), CD262 (NSCLC and other cancers), CD309 (ovarian cancers), CD326 (solid tumors), CEACAM3 (colorectal, gastric cancers), CEACAM5 (carcinoembryonic antigen; CEA, CD66e) (breast, colorectal and lung cancers), DLL3 (delta-like-3), DLL4 (delta-like-4), EGFR (Epidermal Growth Factor Receptor, various cancers), CTLA4 (melanoma), CXCR4 (CD184, Heme-oncology, solid tumors), Endoglin (CD105, solid tumors), EPCAM (epithelial cell adhesion molecule, bladder, head, neck, colon, NHL prostate, and ovarian cancers), ERBB2 (Epidermal Growth Factor Receptor 2; lung, breast, prostate cancers), FCGR1 (autoimmune diseases), FOLR (folate receptor, ovarian cancers), GD2 ganglioside (cancers), G-28 (a cell surface antigen glyvolipid, melanoma), GD3 idiotype (cancers), Heat shock proteins (cancers), HER1 (lung, stomach cancers), HER2 (breast, lung and ovarian cancers), HLA-DR10 (NHL), HLA-DRB (NHL, B cell leukemia), human chorionic gonadotropin (carcinoma), IGF1R (insulin-like growth factor 1 receptor, solid tumors, blood cancers), IL-2 receptor (interleukin 2 receptor, T-cell leukemia and lymphomas), IL-6R (interleukin 6 receptor, multiple myeloma, RA, Castleman's disease, IL6 dependent tumors), Integrins (αvβ3, α5β1, α6β4, αllβ3, α5β5, αvβ5, for various cancers), MAGE-1 (carcinomas), MAGE-2 (carcinomas), MAGE-3 (carcinomas), MAGE 4 (carcinomas), anti-transferrin receptor (carcinomas), p97 (melanoma), MS4A1 (membrane-spanning 4-domains subfamily A member 1, Non-Hodgkin's B cell lymphoma, leukemia), MUC1 or MUC1-KLH (breast, ovarian, cervix, bronchus and gastrointestinal cancer), MUC16 (CA125) (Ovarian cancers), CEA (colorectal), gp100 (melanoma), MART1 (melanoma), MPG (melanoma), MS4A1 (membrane-spanning 4-domains subfamily A, small cell lung cancers, NHL), Nucleolin, Neu oncogene product (carcinomas), P21 (carcinomas), Paratope of anti-(N-glycolylneuraminic acid, Breast, Melanoma cancers), PLAP-like testicular alkaline phosphatase (ovarian, testicular cancers), PSMA (prostate tumors), PSA (prostate), ROBO4, TAG 72 (tumour associated glycoprotein 72, AML, gastric, colorectal, ovarian cancers), T cell transmembrane protein (cancers), Tie (CD202b), TNFRSF10B (tumor necrosis factor receptor superfamily member 10B, cancers), TNFRSF13B (tumor necrosis factor receptor superfamily member 13B, multiple myeloma, NHL, other cancers, RA and SLE), TPBG (trophoblast glycoprotein, Renal cell carcinoma), TRAIL-R1 (Tumor necrosis apoprosis Inducing ligand Receptor 1, lymphoma, NHL, colorectal, lung cancers), VCAM-1 (CD106, Melanoma), VEGF, VEGF-A, VEGF-2 (CD309) (various cancers). Some other tumor associated antigens recognized by antibodies have been reviewed (Gerber, et al, mAbs 1:3, 247-53 (2009); Novellino et al, Cancer Immunol Immunother. 54(3), 187-207 (2005). Franke, et al, Cancer Biother Radiopharm. 2000, 15, 459-76).

The cell-binding agents, more preferred antibodies, can be any agents that are able to against tumor cells, virus infected cells, microorganism infected cells, parasite infected cells, autoimmune cells, activated cells, myeloid cells, activated T-cells, B cells, or melanocytes. More specifically the cell binding agents can be any agent/molecule that is able to against any one of the following antigens or receptors: CD2, CD2R, CD3, CD3gd, CD3e, CD4, CD5, CD6, CD7, CD8, CD8a, CD8b, CD9, CD10, CD11a, CD11b, CD11c, CD12, CD12w, CD13, CD14, CD15, CD15s, CD15u, CD16, CD16a, CD16b, CD17, CDw17, CD18, CD19, CD20, CD21, CD22, CD23, CD24, CD25, CD26, CD27, CD28, CD29, CD30, CD31, CD32, CD33, CD34, CD35, CD36, CD37, CD38, CD39, CD40, CD41, CD42, CD42a, CD42b, CD42c, CD42d, CD43, CD44, CD44R, CD45, CD45RA, CD45RB, CD45RO, CD46, CD47, CD47R, CD48, CD49a, CD49b, CD49c, CD49e, CD49f, CD50, CD51, CD52, CD53, CD54, CD55, CD56, CD57, CD58, CD59, CD60, CD60a, CD60b, CD60c, CD61, CD62E, CD62L, CD62P, CD63, CD64, CD65, CD65s, CD66, CD66a, CD66b, CD66c, CD66d, CD66e, CD66f, CD67, CD68, CD69, CD70, CD71, CD72, CD73, CD74, CD74, CD75, CD75s, CD76, CD77, CD78, CD79, CD79a, CD79b, CD80, CD81, CD82, CD83, CD84, CDw84, CD85, CD86, CD87, CD88, CD89, CD90, CD91, CD92, CDw92, CD93, CD94, CD95, CD96, CD97, CD98, CD99, CD99R, CD100, CD101, CD102, CD103, CD104, CD105, CD106, CD107, CD107a, CD107b, CD108, CD109, CD110, CD111, CD112, CD113, CDw113, CD114, CD115, CD116, CD117, CD118, CD119, CDw119, CD120a, CD120b, CD121a, CD121b, CDw121b, CD122, CD123, CDw123, CD124, CD125, CDw125, CD126, CD127, CD128, CDw128, CD129, CD130, CD131, CDw131, CD132, CD133, CD134, CD135, CD136, CDw136, CD137, CDw137, CD138, CD139, CD140a, CD140b, CD141, CD142, CD143, CD144, CD145, CDw145, CD146, CD147, CD148, CD149, CD150, CD151, CD152, CD153, CD154, CD155, CD156a, CD156b, CDw156c, CD157, CD158a, CD158b, CD159a, CD159b, CD159c, CD160, CD161, CD162, CD162R, CD163, CD164, CD165, CD166, CD167, CD167a, CD168, CD169, CD170, CD171, CD172a, CD172b, CD172g, CD173, CD174, CD175, CD175s, CD176, CD177, CD178, CD179, CD180, CD181, CD182, CD183, CD184, CD185, CD186, CDw186, CD187, CD188, CD189, CD190, Cd191, CD192, CD193, CD194, CD195, CD196, CD197, CD198, CDw198, CD199, CDw199, CD200, CD200a, CD200b, CD201, CD202, CD202b, CD203, CD203c, CD204, CD205, CD206, CD207, CD208, CD209, CD210, CDw210, CD212, CD213a1, CD213a2, CDw217, CDw218a, CDw218b, CD220, CD221, CD222, CD223, CD224, CD225, CD226, CD227, CD228, CD229, CD230, CD231, CD232, CD233, CD234, CD235a, CD235ab, CD235b, CD236, CD236R, CD238, CD239, CD240, CD240CE, CD240D, CD241, CD242, CD243, CD244, CD245, CD246, CD247, CD248, CD249, CD252, CD253, CD254, CD256, CD257, CD258, CD261, CD262, CD263, CD265, CD266, CD267, CD268, CD269, CD271, CD273, CD274, CD275, CD276 (B7-H3), CD277, CD278, CD279, CD280, CD281, CD282, CD283, CD284, CD289, CD292, CDw293, CD294, CD295, CD296, CD297, CD298, CD299, CD300a, CD300c, CD300e, CD301, CD302, CD303, CD304, CD305, CD306, CD309, CD312, CD314, CD315, CD316, CD317, CD318, CD319, CD320, CD321, CD322, CD324, CDw325, CD326, CDw327, CDw328, CDw329, CD331, CD332, CD333, CD334, CD335, CD336, CD337, CDw338, CD339, 4-1BB, 5AC, 5T4 (Trophoblast glycoprotein, TPBG, 5T4, Wnt-Activated Inhibitory Factor 1 or WAIF 1), Adenocarcinomaantigen, AGS-5, AGS-22M6, Activin receptor-like kinase 1, AFP, AKAP-4, ALK, Alpha intergrin, Alpha v beta6, Amino-peptidase N, Amyloid beta, Androgen receptor, Angiopoietin 2, Angiopoietin 3, Annexin A1, Anthrax toxin-protective antigen, Anti-transferrin receptor, AOC3 (VAP-1), B7-H3, Bacillus anthracis anthrax, BAFF (B-cell activating factor), B-lymphoma cell, bcr-abl, Bombesin, BORIS, C5, C242 antigen, CA125 (carbohydrate antigen 125, MUC16), CA-IX (or CAIX, carbonic anhydrase 9), CALLA, CanAg, Canis lupus familiaris IL31, Carbonic anhydrase IX, Cardiac myosin, CCL11 (C—C motif chemokine 11), CCR4 (C—C chemokine receptor type 4, CD194), CCR5, CD3E (epsilon), CEA (Carcinoembryonic antigen), CEACAM3, CEACAM5 (carcinoembryonic antigen), CFD (Factor D), Ch4D5, Cholecystokinin 2 (CCK2R), CLDN18 (Claudin-18), Clumping factor A, CRIPTO, FCSF1R (Colony stimulating factor 1 receptor, CD115), CSF2 (colony stimulating factor 2, Granulocyte-macrophage colony-stimulating factor (GM-CSF)), CTLA4 (cytotoxic T-lymphocyte associated protein 4), CTAA16.88 tumor antigen, CXCR4 (CD184), C—X—C chemokine receptor type 4, cyclic ADP ribose hydrolase, Cyclin B1, CYP1B1, Cytomegalovirus, Cytomegalovirus glycoprotein B, Dabigatran, DLL3 (delta-like-ligand 3), DLL4 (delta-like-ligand 4), DPP4 (Dipeptidyl-peptidase 4), DR5 (Death receptor 5), E. coli shiga toxintype-1, E. coli shiga toxintype-2, ED-B, EGFL7 (EGF-like domain-containing protein 7), EGFR, EGFRII, EGFRvIII, Endoglin (CD105), Endothelin B receptor, Endotoxin, EpCAM (epithelial cell adhesion molecule), EphA2, Episialin, ERBB2 (Epidermal Growth Factor Receptor 2), ERBB3, ERG (TMPRSS2 ETS fusion gene), Escherichia coli, ETV6-AML, FAP (Fibroblast activation proteinalpha), FCGR1, alpha-Fetoprotein, Fibrin II, beta chain, Fibronectin extra domain-B, FOLR (folate receptor), Folate receptor alpha, Folate hydrolase, Fos-related antigen 1, F protein of respiratory syncytial virus, Frizzled receptor, Fucosyl GM1, GD2 ganglioside, G-28 (a cell surface antigen glyvolipid), GD3 idiotype, GloboH, Glypican 3, N-glycolylneuraminic acid, GM3, GMCSF receptor α-chain, Growth differentiation factor 8, GP100, GPNMB (Transmembrane glycoprotein NMB), GUCY2C (Guanylate cyclase 2C, guanylyl cyclase C (GC-C), intestinal Guanylate cyclase, Guanylate cyclase-C receptor, Heat-stable enterotoxin receptor (hSTAR)), Heat shock proteins, Hemagglutinin, Hepatitis B surface antigen, Hepatitis B virus, HER1 (human epidermal growth factor receptor 1), HER2, HER2/neu, HER3 (ERBB-3), IgG4, HGF/SF (Hepatocyte growth factor/scatter factor), HHGFR, HIV-1, Histone complex, HLA-DR (human leukocyte antigen), HLA-DR10, HLA-DRB, HMWMAA, Human chorionic gonadotropin, HNGF, Human scatter factor receptor kinase, HPV E6/E7, Hsp90, hTERT, ICAM-1 (Intercellular Adhesion Molecule 1), Idiotype, IGF1R (IGF-1, insulin-like growth factor 1 receptor), IGHE, IFN-γ, Influeza hemag-glutinin, IgE, IgE Fc region, IGHE, interleukins (e.g. IL-1, IL-2, IL-3, IL-4, IL-5, IL-6, IL-6R, IL-7, IL-8, IL-9, IL-10, IL-11, IL-12, IL-13, IL-15, IL-17, IL-17A, IL-18, IL-19, IL-20, IL-21, IL-22, IL-23, IL-27, or IL-28), IL31RA, ILGF2 (Insulin-like growth factor 2), Integrins (α4, α_{IIb}β₃, αvβ3, α₄β₇, α5β1, α6β4, α7β7, allβ3, α5β5, αvβ5), Interferon gamma-induced protein, ITGA2, ITGB2, KIR2D, LCK, Le, Legumain, Lewis-Y antigen, LFA-1 (Lymphocyte function-associated antigen 1, CD11a), LHRH, LINGO-1, Lipoteichoic acid, LIV1A, LMP2, LTA, MAD-CT-1, MAD-CT-2, MAGE-1, MAGE-2, MAGE-3, MAGE A1, MAGE A3, MAGE 4, MART1, MCP-1, MIF (Macrophage migration inhibitory factor, or glycosylation-inhibiting factor (GIF)), MS4A1 (membrane-spanning 4-domains subfamily A member 1), MSLN (mesothelin), MUC1 (Mucin 1, cell surface associated (MUC1) orpolymorphic epithelial mucin (PEM)), MUC1-KLH, MUC16 (CA125), MCP1 (monocyte chemotactic protein 1), MelanA/MART1, ML-IAP, MPG, MS4A1 (membrane-spanning 4-domains subfamily A), MYCN, Myelin-associated glycoprotein, Myostatin, NA17, NARP-1, NCA-90 (granulocyte antigen), Nectin-4 (ASG-22ME), NGF, Neural apoptosis-regulated proteinase 1, NOGO-A, Notch receptor, Nucleolin, Neu oncogene product, NY-BR-1, NY-ESO-1, OX-40, OxLDL (Oxidized low-density lipoprotein), OY-TES1, P21, p53 nonmutant, P97, Page4, PAP, Paratope of anti-(N-glycolylneuraminic acid), PAX3, PAX5, PCSK9, PDCD1 (PD-1, Programmed cell death protein 1, CD279), PDGF-Rα (Alpha-type platelet-derived growth factor receptor), PDGFR-β, PDL-1, PLAC1, PLAP-like testicular alkaline phosphatase, Platelet-derived growth factor receptor beta, Phosphate-sodium co-transporter, PMEL 17, Polysialic acid, Proteinase3 (PR1), Prostatic carcinoma, PS (Phosphatidylserine), Prostatic carcinoma cells, Pseudomonas aeruginosa, PSMA, PSA, PSCA, Rabies virus glycoprotein, RHD (Rh polypeptide 1 (RhPI), CD240), Rhesus factor, RANKL, RANTES receptors (CCR1, CCR3, CCR5), RhoC, Ras mutant, RGS5, ROBO4, Respiratory syncytial virus, RON, Sarcoma translocation breakpoints, SART3, Sclerostin, SLAMF7 (SLAM family member 7), Selectin P, SDC1 (Syndecan 1), sLe(a), Somatomedin C, SIP (Sphingosine-1-phosphate), Somatostatin, Sperm protein 17, SSX2, STEAP1 (six-transmembrane epithelial antigen of the prostate 1), STEAP2, STn, TAG-72 (tumor associated glycoprotein 72), Survivin, T-cell receptor, T cell transmembrane protein, TEM1 (Tumor endothelial marker 1), TENB2, Tenascin C (TN-C), TGF-α, TGF-β (Transforming growth factor beta), TGF-β1, TGF-β2 (Transforming growth factor-beta 2), Tie (CD202b), Tie2, TIM-1 (CDX-014), Tn, TNF, TNF-α, TNFRSF8, TNFRSF10B (tumor necrosis factor receptor superfamily member 10B), TNFRSF13B (tumor necrosis factor receptor superfamily member 13B), TPBG (trophoblast glycoprotein), TRAIL-R1 (Tumor necrosis apoprosis Inducing ligand Receptor 1), TRAILR2 (Death receptor 5 (DR5)), tumor-associated calcium signal transducer 2, tumor specific glycosylation of MUC1, TWEAK receptor, TYRP1 (glycoprotein 75), TROP-2, TRP-2, Tyrosinase, VCAM-1 (CD106), VEGF, VEGF-A, VEGF-2 (CD309), VEGFR-1, VEGFR2, or vimentin, WT1, XAGE 1, or cells expressing any insulin growth factor receptors, or any epidermal growth factor receptors.

In another specific embodiment, the cell-binding ligand-drug conjugates via the bridge linkers of this invention are used for the targeted treatment of cancers. The targeted cancers include, but are not limited, Adrenocortical Carcinoma, Anal Cancer, Bladder Cancer, Brain Tumor (Adult, Brain Stem Glioma, Childhood, Cerebellar Astrocytoma, Cerebral Astrocytoma, Ependymoma, Medulloblastoma, Supratentorial Primitive Neuroectodermal and Pineal Tumors, Visual Pathway and Hypothalamic Glioma), Breast Cancer, Carcinoid Tumor, Gastrointestinal, Carcinoma of Unknown Primary, Cervical Cancer, Colon Cancer, Endometrial Cancer, Esophageal Cancer, Extrahepatic Bile Duct Cancer, Ewings Family of Tumors (PNET), Extracranial Germ Cell Tumor, Eye Cancer, Intraocular Melanoma, Gallbladder Cancer, Gastric Cancer (Stomach), Germ Cell Tumor, Extragonadal, Gestational Trophoblastic Tumor, Head and Neck Cancer, Hypopharyngeal Cancer, Islet Cell Carcinoma, Kidney Cancer (renal cell cancer), Laryngeal Cancer, Leukemia (Acute Lymphoblastic, Acute Myeloid, Chronic Lymphocytic, Chronic Myelogenous, Hairy Cell), Lip and Oral Cavity Cancer, Liver Cancer, Lung Cancer (Non-Small Cell, Small Cell, Lymphoma (AIDS-Related, Central Nervous System, Cutaneous T-Cell, Hodgkin's Disease, Non-Hodgkin's Disease, Malignant Mesothelioma, Melanoma, Merkel Cell Carcinoma, Metasatic Squamous Neck Cancer with Occult Primary, Multiple Myeloma, and Other Plasma Cell Neoplasms, Mycosis Fungoides, Myelodysplastic Syndrome, Myeloproli-ferative Disorders, Nasopharyngeal Cancer, Neuroblastoma, Oral Cancer, Oropharyngeal Cancer, Osteosarcoma, Ovarian Cancer (Epithelial, Germ Cell Tumor, Low Malignant Potential Tumor), Pancreatic Cancer (Exocrine, Islet Cell Carcinoma), Paranasal Sinus and Nasal Cavity Cancer, Parathyroid Cancer, Penile Cancer, Pheochromocytoma Cancer, Pituitary Cancer, Plasma Cell Neoplasm, Prostate Cancer Rhabdomyosarcoma, Rectal Cancer, Renal Cell Cancer (kidney cancer), Renal Pelvis and Ureter (Transitional Cell), Salivary Gland Cancer, Sezary Syndrome, Skin Cancer, Skin Cancer (Cutaneous T-Cell Lymphoma, Kaposi's Sarcoma, Melanoma), Small Intestine Cancer, Soft Tissue Sarcoma, Stomach Cancer, Testicular Cancer, Thymoma (Malignant), Thyroid Cancer, Urethral Cancer, Uterine Cancer (Sarcoma), Unusual Cancer of Childhood, Vaginal Cancer, Vulvar Cancer, Wilms' Tumor.

In another specific embodiment, the cell-binding-drug conjugates of this invention are used in accordance with the compositions and methods for the treatment or prevention of an autoimmune disease. The autoimmune diseases include, but are not limited, Achlorhydra Autoimmune Active Chronic Hepatitis, Acute Disseminated Encephalomyelitis, Acute hemorrhagic leukoencephalitis, Addison's Disease, Agammaglobulinemia, Alopecia areata, Amyotrophic Lateral Sclerosis, Ankylosing Spondylitis, Anti-GBM/TBM Nephritis, Antiphospholipid syndrome, Antisynthetase syndrome, Arthritis, Atopic allergy, Atopic Dermatitis, Autoimmune Aplastic Anemia, Autoimmune cardiomyopathy, Autoimmune hemolytic anemia, Autoimmune hepatitis, Autoimmune inner ear disease, Autoimmune lymphoproliferative syndrome, Autoimmune peripheral neuropathy, Autoimmune pancreatitis, Autoimmune polyendocrine syndrome Types I, II, & III, Autoimmune progesterone dermatitis, Autoimmune thrombocytopenic purpura, Autoimmune uveitis, Balo disease/Balo concentric sclerosis, Bechets Syndrome, Berger's disease, Bickerstaffs encephalitis, Blau syndrome, Bullous Pemphigoid, Castleman's disease, Chagas disease, Chronic Fatigue Immune Dysfunction Syndrome, Chronic inflammatory demyelinating polyneuropathy, Chronic recurrent multifocal ostomyelitis, Chronic lyme disease, Chronic obstructive pulmonary disease, Churg-Strauss syndrome, Cicatricial Pemphigoid, Coeliac Disease, Cogan syndrome, Cold agglutinin disease, Complement component 2 deficiency, Cranial arteritis, CREST syndrome, Crohns Disease (a type of idiopathic inflammatory bowel diseases), Cushing's Syndrome, Cutaneous leukocytoclastic angiitis, Dego's disease, Dercum's disease, Dermatitis herpetiformis, Dermatomyositis, Diabetes mellitus type 1, Diffuse cutaneous systemic sclerosis, Dressler's syndrome, Discoid lupus erythematosus, Eczema, Endometriosis, Enthesitis-related arthritis, Eosinophilic fasciitis, Epidermolysis bullosa acquisita, Erythema nodosum, Essential mixed cryoglobulinemia, Evan's syndrome, Fibrodysplasia ossificans progressiva, Fibromyalgia, Fibromyositis, Fibrosing aveolitis, Gastritis, Gastrointestinal pemphigoid, Giant cell arteritis, Glomerulonephritis, Goodpasture's syndrome, Graves' disease, Guillain-Barre syndrome, Hashimoto's encephalitis, Hashimoto's thyroiditis, Haemolytic anaemia, Henoch-Schonlein purpura, Herpes gestationis, Hidradenitis suppurativa, Hughes syndrome (See Antiphospholipid syndrome), Hypogamma-globulinemia, Idiopathic Inflammatory Demyelinating Diseases, Idiopathic pulmonary fibrosis, Idiopathic thrombocytopenic purpura (See Autoimmune thrombocytopenic purpura), IgA nephropathy (Also Berger's disease), Inclusion body myositis, Inflammatory demyelinating polyneuopathy, Interstitial cystitis, Irritable Bowel Syndrome, Juvenile idiopathic arthritis, Juvenile rheumatoid arthritis, Kawasaki's Disease, Lambert-Eaton myasthenic syndrome, Leukocytoclastic vasculitis, Lichen planus, Lichen sclerosus, Linear IgA disease (LAD), Lou Gehrig's Disease (Also Amyotrophic lateral sclerosis), Lupoid hepatitis, Lupus erythematosus, Majeed syndrome, Meniere's disease, Microscopic polyangiitis, Miller-Fisher syndrome, Mixed Connective Tissue Disease, Morphea, Mucha-Habermann disease, Muckle-Wells syndrome, Multiple Myeloma, Multiple Sclerosis, Myasthenia gravis, Myositis, Narcolepsy, Neuromyelitis optica (Devic's Disease), Neuromyotonia, Occular cicatricial pemphigoid, Opsoclonus myoclonus syndrome, Ord thyroiditis, Palindromic rheumatism, PANDAS (Pediatric Autoimmune Neuropsychiatric Disorders Associated with Streptococcus), Paraneoplastic cerebellar degeneration, Paroxysmal nocturnal hemoglobinuria, Parry Romberg syndrome, Parsonnage-Turner syndrome, Pars planitis, Pemphigus, Pemphigus vulgaris, Pernicious anaemia, Perivenous encephalomyelitis, POEMS syndrome, Polyarteritis nodosa, Polymyalgia rheumatica, Polymyositis, Primary biliary cirrhosis, Primary sclerosing cholangitis, Progressive inflammatory neuropathy, Psoriasis, Psoriatic Arthritis, Pyoderma gangrenosum, Pure red cell aplasia, Rasmussen's encephalitis, Raynaud phenomenon, Relapsing polychondritis, Reiter's syndrome, Restless leg syndrome, Retroperitoneal fibrosis, Rheumatoid arthritis, Rheumatoid fever, Sarcoidosis, Schizophrenia, Schmidt syndrome, Schnitzler syndrome, Scleritis, Scleroderma, Sjögren's syndrome, Spondyloarthropathy, Sticky blood syndrome, Still's Disease, Stiff person syndrome, Subacute bacterial endocarditis, Susac's syndrome, Sweet syndrome, Sydenham Chorea, Sympathetic ophthalmia, Takayasu's arteritis, Temporal arteritis (giant cell arteritis), Tolosa-Hunt syndrome, Transverse Myelitis, Ulcerative Colitis (a type of idiopathic inflammatory bowel diseases), Undifferentiated connective tissue disease, Undifferentiated spondyloarthropathy, Vasculitis, Vitiligo, Wegener's granulomatosis, Wilson's syndrome, Wiskott-Aldrich syndrome

In another specific embodiment, a binding molecule used for the conjugate via the bis-linkers of this invention for the treatment or prevention of an autoimmune disease can be, but are not limited to, anti-elastin antibody; Abys against epithelial cells antibody; Anti-Basement Membrane Collagen Type IV Protein antibody; Anti-Nuclear Antibody; Anti ds DNA; Anti ss DNA, Anti Cardiolipin Antibody IgM, IgG; anti-celiac antibody; Anti Phospholipid Antibody IgK, IgG; Anti SM Antibody; Anti Mitochondrial Antibody; Thyroid Antibody; Microsomal Antibody, T-cells antibody; Thyroglobulin Antibody, Anti SCL-70; Anti-Jo; Anti-U.sub.1RNP; Anti-La/SSB; Anti SSA; Anti SSB; Anti Perital Cells Antibody; Anti Histones; Anti RNP; C-ANCA; P-ANCA; Anti centromere; Anti-Fibrillarin, and Anti GBM Antibody, Anti-ganglioside antibody; Anti-Desmogein 3 antibody; Anti-p62 antibody; Anti-sp100 antibody; Anti-Mitochondrial (M2) antibody; Rheumatoid factor antibody; Anti-MCV antibody; Anti-topoisomerase antibody; Anti-neutrophil cytoplasmic (cANCA) antibody.

In certain preferred embodiments, the binding molecule for the conjugate in the present invention, can bind to both a receptor and a receptor complex expressed on an activated lymphocyte which is associated with an autoimmune disease. The receptor or receptor complex can comprise an immunoglobulin gene superfamily member (e.g. CD2, CD3, CD4, CD8, CD19, CD20, CD22, CD28, CD30, CD33, CD37, CD38, CD56, CD70, CD79, CD79b, CD90, CD125, CD137, CD138, CD147, CD152/CTLA-4, PD-1, or ICOS), a TNF receptor superfamily member (e.g. CD27, CD40, CD95/Fas, CD134/OX40, CD137/4-1BB, INF-R1, TNFR-2, RANK, TACI, BCMA, osteoprotegerin, Apo2/TRAIL-R1, TRAIL-R2, TRAIL-R3, TRAIL-R4, and APO-3), an integrin, a cytokine receptor, a chemokine receptor, a major histocompatibility protein, a lectin (C-type, S-type, or I-type), or a complement control protein.

In another specific embodiment, useful cell binding ligands that are immunospecific for a viral or a microbial antigen are humanized or human monoclonal antibodies. As used herein, the term “viral antigen” includes, but is not limited to, any viral peptide, polypeptide protein (e.g. HIV gp120, HIV nef, RSV F glycoprotein, influenza virus neuramimi-dase, influenza virus hemagglutinin, HTLV tax, herpes simplex virus glycoprotein (e.g. gB, gC, gD, and gE) and hepatitis B surface antigen) that is capable of eliciting an immune response. As used herein, the term “microbial antigen” includes, but is not limited to, any microbial peptide, polypeptide, protein, saccharide, polysaccharide, or lipid molecule (e.g., a bacteria, fungi, pathogenic protozoa, or yeast polypeptides including, e.g., LPS and capsular polysaccharide ⅝) that is capable of eliciting an immune response. Examples of antibodies available 1 for the viral or microbial infection include, but are not limited to, Palivizumab which is a humanized anti-respiratory syncytial virus monoclonal antibody for the treatment of RSV infection; PR0542 which is a CD4 fusion antibody for the treatment of HIV infection; Ostavir which is a human antibody for the treatment of hepatitis B virus; PROTVIR which is a humanized IgG.sub.1 antibody for the treatment of cytomegalovirus; and anti-LPS antibodies.

The cell binding molecules-drug conjugates via the bis-linkers of this invention can be used in the treatment of infectious diseases. These infectious diseases include, but are not limited to, Acinetobacter infections, Actinomycosis, African sleeping sickness (African trypanosomiasis), AIDS (Acquired immune deficiency syndrome), Amebiasis, Anaplasmosis, Anthrax, Arcano-bacterium haemolyticum infection, Argentine hemorrhagic fever, Ascariasis, Aspergillosis, Astrovirus infection, Babesiosis, Bacillus cereus infection, Bacterial pneumonia, Bacterial vaginosis, Bacteroides infection, Balantidiasis, Baylisascaris infection, BK virus infection, Black piedra, Blastocystis hominis infection, Blastomycosis, Bolivian hemorrhagic fever, Borrelia infection, Botulism (and Infant botulism), Brazilian hemorrhagic fever, Brucellosis, Burkholderia infection, Buruli ulcer, Calicivirus infection (Norovirus and Sapovirus), Campylobacteriosis, Candidiasis (Moniliasis; Thrush), Cat-scratch disease, Cellulitis, Chagas Disease (American trypanosomiasis), Chancroid, Chickenpox, Chlamydia, Chlamydophila pneumoniae infection, Cholera, Chromoblastomycosis, Clonorchiasis, Clostridium difficile infection, Coccidioido-mycosis, Colorado tick fever, Common cold (Acute viral rhinopharyngitis; Acute coryza), Creutzfeldt-Jakob disease, Crimean-Congo hemorrhagic fever, Cryptococcosis, Cryptosporidiosis, Cutaneous larva migrans, Cyclosporiasis, Cysticercosis, Cytomegalovirus infection, Dengue fever, Dientamoebiasis, Diphtheria, Diphyllobothriasis, Dracunculiasis, Ebola hemorrhagic fever, Echinococcosis, Ehrlichiosis, Enterobiasis (Pinworm infection), Enterococcus infection, Enterovirus infection, Epidemic typhus, Erythema infectiosum (Fifth disease), Exanthem subitum, Fasciolopsiasis, Fasciolosis, Fatal familial insomnia, Filariasis, Food poisoning by Clostridium perfringens, Free-living amebic infection, Fusobacterium infection, Gas gangrene (Clostridial myonecrosis), Geotrichosis, Gerstmann-Straussler-Scheinker syndrome, Giardiasis, Glanders, Gnathosto-miasis, Gonorrhea, Granuloma inguinale (Donovanosis), Group A streptococcal infection, Group B streptococcal infection, Haemophilus influenzae infection, Hand, foot and mouth disease (HFMD), Hantavirus Pulmonary Syndrome, Helicobacter pylori infection, Hemolytic-uremic syndrome, Hemorrhagic fever with renal syndrome, Hepatitis A, Hepatitis B, Hepatitis C, Hepatitis D, Hepatitis E, Herpes simplex, Histoplasmosis, Hookworm infection, Human bocavirus infection, Human ewingii ehrlichiosis, Human granulocytic anaplasmosis, Human metapneumovirus infection, Human monocytic ehrlichiosis, Human papillomavirus infection, Human parainfluenza virus infection, Hymenolepiasis, Epstein-Barr Virus Infectious Mononucleosis (Mono), Influenza, Isosporiasis, Kawasaki disease, Keratitis, Kingella kingae infection, Kuru, Lassa fever, Legionellosis (Legionnaires' disease), Legionellosis (Pontiac fever), Leishmaniasis, Leprosy, Leptospirosis, Listeriosis, Lyme disease (Lyme borreliosis), Lymphatic filariasis (Elephantiasis), Lymphocytic choriomeningitis, Malaria, Marburg hemorrhagic fever, Measles, Melioidosis (Whitmore's disease), Meningitis, Meningococcal disease, Metagonimiasis, Microsporidiosis, Molluscum contagiosum, Mumps, Murine typhus (Endemic typhus), Mycoplasma pneumonia, Mycetoma, Myiasis, Neonatal conjunctivitis (Ophthalmia neonatorum), (New) Variant Creutzfeldt-Jakob disease (vCJD, nvCJD), Nocardiosis, Onchocerciasis (River blindness), Paracoccidioidomycosis (South American blastomycosis), Paragonimiasis, Pasteurellosis, Pediculosis capitis (Head lice), Pediculosis corporis (Body lice), Pediculosis pubis (Pubic lice, Crab lice), Pelvic inflammatory disease, Pertussis (Whooping cough), Plague, Pneumococcal infection, Pneumocystis pneumonia, Pneumonia, Poliomyelitis, Prevotella infection, Primary amoebic meningoencephalitis, Progressive multifocal leukoencephalopathy, Psittacosis, Q fever, Rabies, Rat-bite fever, Respiratory syncytial virus infection, Rhinosporidiosis, Rhinovirus infection, Rickettsial infection, Rickettsial-pox, Rift Valley fever, Rocky mountain spotted fever, Rotavirus infection, Rubella, Salmonellosis, SARS (Severe Acute Respiratory Syndrome), Scabies, Schistosomiasis, Sepsis, Shigellosis (Bacillary dysentery), Shingles (Herpes zoster), Smallpox (Variola), Sporotrichosis, Staphylococcal food poisoning, Staphylococcal infection, Strongyloidiasis, Syphilis, Taeniasis, Tetanus (Lockjaw), Tinea barbae (Barber's itch), Tinea capitis (Ringworm of the Scalp), Tinea corporis (Ringworm of the Body), Tinea cruris (Jock itch), Tinea manuum (Ringworm of the Hand), Tinea nigra, Tinea pedis (Athlete's foot), Tinea unguium (Onychomycosis), Tinea versicolor (Pityriasis versicolor), Toxocariasis (Ocular Larva Migrans), Toxocariasis (Visceral Larva Migrans), Toxoplasmosis, Trichinellosis, Trichomoniasis, Trichuriasis (Whipworm infection), Tuberculosis, Tularemia, Ureaplasma urealyticum infection, Venezuelan equine encephalitis, Venezuelan hemorrhagic fever, Viral pneumonia, West Nile Fever, White piedra (Tinea blanca), Yersinia pseudotuber-culosis infection, Yersiniosis, Yellow fever, Zygomycosis.

The cell binding molecule, which is more preferred to be an antibody described in this patent that are against pathogenic strains include, but are not limit, Acinetobacter baumannii, Actinomyces israelii, Actinomyces gerencseriae and Propionibacterium propionicus, Trypanosoma brucei, HIV (Human immunodeficiency virus), Entamoeba histolytica, Anaplasma genus, Bacillus anthracis, Arcanobacterium haemolyticum, Junin virus, Ascaris lumbricoides, Aspergillus genus, Astroviridae family, Babesia genus, Bacillus cereus, multiple bacteria, Bacteroides genus, Balantidium coli, Baylisascaris genus, BK virus, Piedraia hortae, Blastocystis hominis, Blastomyces dermatitides, Machupo virus, Borrelia genus, Clostridium botulinum, Sabia, Brucella genus, usually Burkholderia cepacia and other Burkholderia species, Mycobacterium ulcerans, Caliciviridae family, Campylobacter genus, usually Candida albicans and other Candida species, Bartonella henselae, Group A Streptococcus and Staphylococcus, Trypanosoma cruzi, Haemophilus ducreyi, Varicella zoster virus (VZV), Chlamydia trachomatis, Chlamydophila pneumoniae, Vibrio cholerae, Fonsecaea pedrosoi, Clonorchis sinensis, Clostridium difficile, Coccidioides immitis and Coccidioides posadasii, Colorado tick fever virus, rhinoviruses, coronaviruses, CJD prion, Crimean-Congo hemorrhagic fever virus, Cryptococcus neoformans, Cryptosporidium genus, Ancylostoma braziliense; multiple parasites, Cyclospora cayetanensis, Taenia solium, Cytomegalovirus, Dengue viruses (DEN-1, DEN-2, DEN-3 and DEN-4)—Flaviviruses, Dientamoeba fragilis, Corynebacterium diphtheriae, Diphyllobothrium, Dracunculus medinensis, Ebolavirus, Echinococcus genus, Ehrlichia genus, Enterobius vermicularis, Enterococcus genus, Enterovirus genus, Rickettsia prowazekii, Parvovirus B19, Human herpesvirus 6 and Human herpesvirus 7, Fasciolopsis buski, Fasciola hepatica and Fasciola gigantica, FFI prion, Filarioidea superfamily, Clostridium perfringens, Fusobacterium genus, Clostridium perfringens; other Clostridium species, Geotrichum candidum, GSS prion, Giardia intestinalis, Burkholderia mallei, Gnathostoma spinigerum and Gnathostoma hispidum, Neisseria gonorrhoeae, Klebsiella granulomatis, Streptococcus pyogenes, Streptococcus agalactiae, Haemophilus influenzae, Enteroviruses, mainly Coxsackie A virus and Enterovirus 71, Sin Nombre virus, Helicobacter pylori, Escherichia coli O157:H7, Bunyaviridae family, Hepatitis A Virus, Hepatitis B Virus, Hepatitis C Virus, Hepatitis D Virus, Hepatitis E Virus, Herpes simplex virus 1, Herpes simplex virus 2, Histoplasma capsulatum, Ancylostoma duodenale and Necator americanus, Hemophilus influenzae, Human bocavirus, Ehrlichia ewingii, Anaplasma phagocytophilum, Human metapneumovirus, Ehrlichia chaffeensis, Human papillomavirus, Human parainfluenza viruses, Hymenolepis nana and Hymenolepis diminuta, Epstein-Barr Virus, Orthomy-xoviridae family, Isospora belli, Kingella kingae, Klebsiella pneumoniae, Klebsiella ozaenas, Klebsiella rhinoscleromotis, Kuru prion, Lassa virus, Legionella pneumophila, Legionella pneumophila, Leishmania genus, Mycobacterium leprae and Mycobacterium lepromatosis, Leptospira genus, Listeria monocytogenes, Borrelia burgdorferi and other Borrelia species, Wuchereria bancrofti and Brugia malayi, Lymphocytic choriomeningitis virus (LCMV), Plasmodium genus, Marburg virus, Measles virus, Burkholderia pseudomallei, Neisseria meningitides, Metagonimus yokagawai, Microsporidia phylum, Molluscum contagiosum virus (MCV), Mumps virus, Rickettsia typhi, Mycoplasma pneumoniae, numerous species of bacteria (Actinomycetoma) and fungi (Eumycetoma), parasitic dipterous fly larvae, Chlamydia trachomatis and Neisseria gonorrhoeae, vCJD prion, Nocardia asteroides and other Nocardia species, Onchocerca volvulus, Paracoccidioides brasiliensis, Paragonimus westermani and other Paragonimus species, Pasteurella genus, Pediculus humanus capitis, Pediculus humanus corporis, Phthirus pubis, Bordetella pertussis, Yersinia pestis, Streptococcus pneumoniae, Pneumocystis jirovecii, Poliovirus, Prevotella genus, Naegleria fowleri, JC virus, Chlamydophila psittaci, Coxiella burnetii, Rabies virus, Streptobacillus moniliformis and Spirillum minus, Respiratory syncytial virus, Rhinosporidium seeberi, Rhinovirus, Rickettsia genus, Rickettsia akari, Rift Valley fever virus, Rickettsia rickettsii, Rotavirus, Rubella virus, Salmonella genus, SARS coronavirus, Sarcoptes scabiei, Schistosoma genus, Shigella genus, Varicella zoster virus, Variola major or Variola minor, Sporothrix schenckii, Staphylococcus genus, Staphylococcus genus, Staphylococcus aureus, Streptococcus pyogenes, Strongyloides stercoralis, Treponema pallidum, Taenia genus, Clostridium tetani, Trichophyton genus, Trichophyton tonsurans, Trichophyton genus, Epidermophyton floccosum, Trichophyton rubrum, and Trichophyton mentagrophytes, Trichophyton rubrum, Hortaea werneckii, Trichophyton genus, Malassezia genus, Toxocara canis or Toxocara cati, Toxoplasma gondii, Trichinella spiralis, Trichomonas vaginalis, Trichuris trichiura, Mycobacterium tuberculosis, Francisella tularensis, Ureaplasma urealyticum, Venezuelan equine encephalitis virus, Vibrio colerae, Guanarito virus, West Nile virus, Trichosporon beigelii, Yersinia pseudotuberculosis, Yersinia enterocolitica, Yellow fever virus, Mucorales order (Mucormycosis) and Entomophthorales order (Entomophthora-mycosis), Pseudomonas aeruginosa, Campylobacter (Vibrio) fetus, Aeromonas hydrophila, Edwardsiella tarda, Yersinia pestis, Shigella dysenteriae, Shigella flexneri, Shigella sonnei, Salmonella typhimurium, Treponema pertenue, Treponema carateneum, Borrelia vincentii, Borrelia burgdorferi, Leptospira icterohemorrhagiae, Pneumocystis carinii, Brucella abortus, Brucella suis, Brucella melitensis, Mycoplasma spp., Rickettsia prowazeki, Rickettsia tsutsugumushi, Clamydia spp.; pathogenic fungi (Aspergillus fumigatus, Candida albicans, Histoplasma capsulatum); protozoa (Entomoeba histolytica, Trichomonas tenas, Trichomonas hominis, Tryoanosoma gambiense, Trypanosoma rhodesiense, Leishmania donovani, Leishmania tropica, Leishmania braziliensis, Pneumocystis pneumonia, Plasmodium vivax, Plasmodium falciparum, Plasmodium malaria); or Helminiths (Schistosoma japonicum, Schistosoma mansoni, Schistosoma haematobium, and hookworms).

Other antibodies as cell binding ligands used in this invention for treatment of viral disease include, but are not limited to, antibodies against antigens of pathogenic viruses, including as examples and not by limitation: Poxyiridae, Herpesviridae, Adenoviridae, Papovaviridae, Enteroviridae, Picornaviridae, Parvoviridae, Reoviridae, Retroviridae, influenza viruses, parainfluenza viruses, mumps, measles, respiratory syncytial virus, rubella, Arboviridae, Rhabdoviridae, Arenaviridae, Non-A/Non-B Hepatitis virus, Rhinoviridae, Coronaviridae, Rotoviridae, Oncovirus [such as, HBV (Hepatocellular carcinoma), HPV (Cervical cancer, Anal cancer), Kaposi's sarcoma-associated herpesvirus (Kaposi's sarcoma), Epstein-Barr virus (Nasopharyngeal carcinoma, Burkitt's lymphoma, Primary central nervous system lymphoma), MCPyV (Merkel cell cancer), SV40 (Simian virus 40), HCV (Hepatocellular carcinoma), HTLV-I (Adult T-cell leukemia/lymphoma)], Immune disorders caused virus: [such as Human Immunodeficiency Virus (AIDS)]; Central nervous system virus: [such as, JCV (Progressive multifocal leukoencephalopathy), MeV (Subacute sclerosing panencephalitis), LCV (Lymphocytic choriomeningitis), Arbovirus encephalitis, Orthomyxoviridae (probable) (Encephalitis lethargica), RV (Rabies), Chandipura virus, Herpesviral meningitis, Ramsay Hunt syndrome type II; Poliovirus (Poliomyelitis, Post-polio syndrome), HTLV-I (Tropical spastic paraparesis)]; Cytomegalovirus (Cytomegalovirus retinitis, HSV (Herpetic keratitis)); Cardiovascular virus [such as CBV (Pericarditis, Myocarditis)]; Respiratory system/acute viral nasopharyngitis/viral pneumonia: [Epstein-Barr virus (EBV infection/Infectious mononucleosis), Cytomegalovirus; SARS coronavirus (Severe acute respiratory syndrome) Orthomyxoviridae: Influenzavirus A/B/C (Influenza/Avian influenza), Paramyxovirus: Human parainfluenza viruses (Parainfluenza), RSV (Human respiratory syncytialvirus), hMPV]; Digestive system virus [MuV (Mumps), Cytomegalovirus (Cytomegalovirus esophagitis); Adenovirus (Adenovirus infection); Rotavirus, Norovirus, Astrovirus, Coronavirus; HBV (Hepatitis B virus), CBV, HAV (Hepatitis A virus), HCV (Hepatitis C virus), HDV (Hepatitis D virus), HEV (Hepatitis E virus), HGV (Hepatitis G virus)]; Urogenital virus [such as, BK virus, MuV (Mumps)].

According to a further object, the present invention also concerns pharmaceutical compositions comprising the conjugate of the invention together with a pharmaceutically acceptable carrier, diluent, or excipient for treatment of cancers, infections or autoimmune disorders. The method for treatment of cancers, infections and autoimmune disorders can be practiced in vitro, in vivo, or ex vivo. Examples of in vitro uses include treatments of cell cultures in order to kill all cells except for desired variants that do not express the target antigen; or to kill variants that express undesired antigen. Examples of ex vivo uses include treatments of hematopoietic stem cells (HSC) prior to the performance of the transplantation (HSCT) into the same patient in order to kill diseased or malignant cells. For instance, clinical ex vivo treatment to remove tumour cells or lymphoid cells from bone marrow prior to autologous transplantation in cancer treatment or in treatment of autoimmune disease, or to remove T cells and other lymphoid cells from allogeneic bone marrow or tissue prior to transplant in order to prevent graft-versus-host disease, can be carried out as follows. Bone marrow is harvested from the patient or other individual and then incubated in medium containing serum to which is added the conjugate of the invention, concentrations range from about 1 pM to 0.1 mM, for about 30 minutes to about 48 hours at about 37° C. The exact conditions of concentration and time of incubation (=dose) are readily determined by the skilled clinicians. After incubation, the bone marrow cells are washed with medium containing serum and returned to the patient by i.v. infusion according to known methods. In circumstances where the patient receives other treatment such as a course of ablative chemotherapy or total-body irradiation between the time of harvest of the marrow and reinfusion of the treated cells, the treated marrow cells are stored frozen in liquid nitrogen using standard medical equipment.

Drugs/Cytotoxic Agents for Conjugation

Drugs that can be conjugated to a cell-binding molecule in the present invention are small molecule drugs including cytotoxic agents, which can be linked to or after they are modified for linkage to the cell-binding agent. A “small molecule drug” is broadly used herein to refer to an organic, inorganic, or organometallic compound that may have a molecular weight of, for example, 100 to 2500, more suitably from 200 to 2000. Small molecule drugs are well characterized in the art, such as in WO05058367A2, and in U.S. Pat. No. 4,956,303, among others and are incorporated in their entirety by reference. The drugs include known drugs and those that may become known drugs.

Drugs that are known include, but not limited to,

1). Chemotherapeutic agents: a). Alkylating agents: such as Nitrogen mustards: chlorambucil, chlornaphazine, cyclophosphamide, dacarbazine, estramustine, ifosfamide, mechlorethamine, mechlorethamine oxide hydrochloride, mannomustine, mitobronitol, melphalan, mitolactol, pipobroman, novembichin, phenesterine, prednimustine, thiotepa, trofosfamide, uracil mustard; CC-1065 (including its adozelesin, carzelesin and bizelesin synthetic analogues); Duocarmycin (including the synthetic analogues, KW-2189, CBI-TMI, and CBI dimers); Benzodiazepine dimers (e.g., dimers of pyrrolobenzodiazepine (PBD) or tomaymycin, indolinobenzodiazepines, imidazobenzothiadiazepines, or oxazolidinobenzodiazepines); Nitrosoureas: (carmustine, lomustine, chlorozotocin, fotemustine, nimustine, ranimustine); Alkylsulphonates: (busulfan, treosulfan, improsulfan and piposulfan); Triazenes: (dacarbazine); Platinum containing compounds: (carboplatin, cisplatin, oxaliplatin); aziridines, such as benzodopa, carboquone, meturedopa, and uredopa; ethylenimines and methylamelamines including altretamine, triethylenemel-amine, trietylenephosphoramide, triethylenethio-phosphaoramide and trimethylolomel-amine]; b). Plant Alkaloids: such as Vinca alkaloids: (vincristine, vinblastine, vindesine, vinorelbine, navelbin); Taxoids: (paclitaxel, docetaxol) and their analogs, Maytansinoids (DM1, DM2, DM3, DM4, maytansine and ansamitocins) and their analogs, cryptophycins (particularly cryptophycin 1 and cryptophycin 8); epothilones, eleutherobin, discodermolide, bryostatins, dolostatins, auristatins, tubulysins, cephalostatins; pancratistatin; a sarcodictyin; spongistatin; c). DNA Topoisomerase Inhibitors: such as [Epipodophyllins: (9-aminocamptothecin, camptothecin, crisnatol, daunomycin, etoposide, etoposide phosphate, irinotecan, mitoxantrone, novantrone, retinoic acids (retinols), teniposide, topotecan, 9-nitrocamptothecin (RFS 2000)); mitomycins: (mitomycin C) and its analogs]; d). Anti-metabolites: such as {[Anti-folate: DHFR inhibitors: (methotrexate, trimetrexate, denopterin, pteropterin, aminopterin (4-aminopteroic acid) or the other folic acid analogues); IMP dehydrogenase Inhibitors: (mycophenolic acid, tiazofurin, ribavirin, EICAR); Ribonucleotide reductase Inhibitors: (hydroxyurea, deferoxamine)]; [Pyrimidine analogs: Uracil analogs: (ancitabine, azacitidine, 6-azauridine, capecitabine (Xeloda), carmofur, cytarabine, dideoxyuridine, doxifluridine, enocitabine, 5-Fluorouracil, floxuridine, ratitrexed (Tomudex)); Cytosine analogs: (cytarabine, cytosine arabinoside, fludarabine); Purine analogs: (azathioprine, fludarabine, mercaptopurine, thiamiprine, thioguanine)]; folic acid replenisher, such as frolinic acid}; e). Hormonal therapies: such as {Receptor antagonists: [Anti-estrogen: (megestrol, raloxifene, tamoxifen); LHRH agonists: (goscrclin, leuprolide acetate); Anti-androgens: (bicalutamide, flutamide, calusterone, dromostanolone propionate, epitiostanol, goserelin, leuprolide, mepitiostane, nilutamide, testolactone, trilostane and other androgens inhibitors)]; Retinoids/Deltoids: [Vitamin D3 analogs: (CB 1093, EB 1089 KH 1060, cholecalciferol, ergocalciferol); Photodynamic therapies: (verteporfin, phthalocyanine, photosensitizer Pc4, demethoxyhypocrellin A); Cytokines: (Interferon-alpha, Interferon-gamma, tumor necrosis factor (TNFs), human proteins containing a TNF domain)]}; f). Kinase inhibitors, such as BIBW 2992 (anti-EGFR/Erb2), imatinib, gefitinib, pegaptanib, sorafenib, dasatinib, sunitinib, erlotinib, nilotinib, lapatinib, axitinib, pazopanib. vandetanib, E7080 (anti-VEGFR2), mubritinib, ponatinib (AP24534), bafetinib (INNO-406), bosutinib (SKI-606), cabozantinib, vismodegib, iniparib, ruxolitinib, CYT387, axitinib, tivozanib, sorafenib, bevacizumab, cetuximab, Trastuzumab, Ranibizumab, Panitumumab, ispinesib; g). A poly (ADP-ribose) polymerase (PARP) inhibitors, such as olaparib, niraparib, iniparib, talazoparib, veliparib, veliparib, CEP 9722 (Cephalon's), E7016 (Eisai's), BGB-290 (BeiGene's), 3-aminobenzamide.

h). antibiotics, such as the enediyne antibiotics (e.g. calicheamicins, especially calicheamicin γ1, δ1, α1 and β1, see, e.g., J. Med. Chem., 39 (11), 2103-2117 (1996), Angew Chem Intl. Ed. Engl. 33:183-186 (1994); dynemicin, including dynemicin A and deoxydynemicin; esperamicin, kedarcidin, C-1027, maduropeptin, as well as neocarzinostatin chromophore and related chromoprotein enediyne antiobiotic chromomophores), aclacinomysins, actinomycin, authramycin, azaserine, bleomycins, cactinomycin, carabicin, carminomycin, carzinophilin; chromomycins, dactinomycin, daunorubicin, detorubicin, 6-diazo-5-oxo-L-norleucine, doxorubicin, morpholino-doxorubicin, cyanomorpholino-doxorubicin, 2-pyrrolino-doxorubicin and deoxydoxorubicin, epirubicin, esorubicin, idarubicin, marcellomycin, nitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; i). Others: such as Polyketides (acetogenins), especially bullatacin and bullatacinone; gemcitabine, epoxomicins (e. g. carfilzomib), bortezomib, thalidomide, lenalidomide, pomalidomide, tosedostat, zybrestat, PLX4032, STA-9090, Stimuvax, allovectin-7, Xegeva, Provenge, Yervoy, Isoprenylation inhibitors (such as Lovastatin), Dopaminergic neurotoxins (such as 1-methyl-4-phenylpyridinium ion), Cell cycle inhibitors (such as staurosporine), Actinomycins (such as Actinomycin D, dactinomycin), Bleomycins (such as bleomycin A2, bleomycin B2, peplomycin), Anthracyclines (such as daunorubicin, doxorubicin (adriamycin), idarubicin, epirubicin, eribulin, pirarubicin, zorubicin, mtoxantrone, MDR inhibitors (such as verapamil), Ca²⁺ ATPase inhibitors (such as thapsigargin), Histone deacetylase inhibitors (Vorinostat, Romidepsin, Panobinostat, Valproic acid, Mocetinostat (MGCD0103), Belinostat, PCI-24781, Entinostat, SB939, Resminostat, Givinostat, AR-42, CUDC-101, sulforaphane, Trichostatin A); Thapsigargin, Celecoxib, glitazones, epigallocatechin gallate, Disulfiram, Salinosporamide A.; Anti-adrenals, such as aminoglutethimide, mitotane, trilostane; aceglatone; aldophosphamide glycoside; aminolevulinic acid; amsacrine; arabinoside, bestrabucil; bisantrene; edatraxate; defofamine; demecolcine; diaziquone; eflornithine (DFMO), elfomithine; elliptinium acetate, etoglucid; gallium nitrate; gacytosine, hydroxyurea; ibandronate, lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; rhizoxin; sizofiran; spirogermanium; tenuazonic acid; triaziquone; 2, 2′,2″-trichlorotriethylamine; trichothecenes (especially T-2 toxin, verrucarin A, roridin A and anguidine); urethane, siRNA, antisense drugs, and a nucleolytic enzyme.

2). An anti-autoimmune disease agent includes, but is not limited to, cyclosporine, cyclosporine A, aminocaproic acid, azathioprine, bromocriptine, chlorambucil, chloroquine, cyclophosphamide, corticosteroids (e.g. amcinonide, betamethasone, budesonide, hydrocortisone, flunisolide, fluticasone propionate, fluocortolone danazol, dexamethasone, Triamcinolone acetonide, beclometasone dipropionate), DHEA, enanercept, hydroxychloroquine, infliximab, meloxicam, methotrexate, mofetil, mycophenylate, prednisone, sirolimus, tacrolimus.

3). An anti-infectious disease agent includes, but is not limited to, a). Aminoglycosides: amikacin, astromicin, gentamicin (netilmicin, sisomicin, isepamicin), hygromycin B, kanamycin (amikacin, arbekacin, bekanamycin, dibekacin, tobramycin), neomycin (framycetin, paromomycin, ribostamycin), netilmicin, spectinomycin, streptomycin, tobramycin, verdamicin; b). Amphenicols: azidamfenicol, chloramphenicol, florfenicol, thiamphenicol; c). Ansamycins: geldanamycin, herbimycin; d). Carbapenems: biapenem, doripenem, ertapenem, imipenem/cilastatin, meropenem, panipenem; e). Cephems: carbacephem (loracarbef), cefacetrile, cefaclor, cefradine, cefadroxil, cefalonium, cefaloridine, cefalotin or cefalothin, cefalexin, cefaloglycin, cefamandole, cefapirin, cefatrizine, cefazaflur, cefazedone, cefazolin, cefbuperazone, cefcapene, cefdaloxime, cefepime, cefminox, cefoxitin, cefprozil, cefroxadine, ceftezole, cefuroxime, cefixime, cefdinir, cefditoren, cefepime, cefetamet, cefmenoxime, cefodizime, cefonicid, cefoperazone, ceforanide, cefotaxime, cefotiam, cefozopran, cephalexin, cefpimizole, cefpiramide, cefpirome, cefpodoxime, cefprozil, cefquinome, cefsulodin, ceftazidime, cefteram, ceftibuten, ceftiolene, ceftizoxime, ceftobiprole, ceftriaxone, cefuroxime, cefuzonam, cephamycin (cefoxitin, cefotetan, cefmetazole), oxacephem (flomoxef, latamoxef); f). Glycopeptides: bleomycin, vancomycin (oritavancin, telavancin), teicoplanin (dalbavancin), ramoplanin; g). Glycylcyclines: e. g. tigecycline; g). β-Lactamase inhibitors: penam (sulbactam, tazobactam), clavam (clavulanic acid); i). Lincosamides: clindamycin, lincomycin; j). Lipopeptides: daptomycin, A54145, calcium-dependent antibiotics (CDA); k). Macrolides: azithromycin, cethromycin, clarithromycin, dirithromycin, erythromycin, flurithromycin, josamycin, ketolide (telithromycin, cethromycin), midecamycin, miocamycin, oleandomycin, rifamycins (rifampicin, rifampin, rifabutin, rifapentine), rokitamycin, roxithromycin, spectinomycin, spiramycin, tacrolimus (FK506), troleandomycin, telithromycin; l). Monobactams: aztreonam, tigemonam; m). Oxazolidinones: linezolid; n). Penicillins: amoxicillin, ampicillin (pivampicillin, hetacillin, bacampicillin, metampicillin, talampicillin), azidocillin, azlocillin, benzylpenicillin, benzathine benzylpenicillin, benzathine phenoxymethyl-penicillin, clometocillin, procaine benzylpenicillin, carbenicillin (carindacillin), cloxacillin, dicloxacillin, epicillin, flucloxacillin, mecillinam (pivmecillinam), mezlocillin, meticillin, nafcillin, oxacillin, penamecillin, penicillin, pheneticillin, phenoxymethylpenicillin, piperacillin, propicillin, sulbenicillin, temocillin, ticarcillin; o). Polypeptides: bacitracin, colistin, polymyxin B; p). Quinolones: alatrofloxacin, balofloxacin, ciprofloxacin, clinafloxacin, danofloxacin, difloxacin, enoxacin, enrofloxacin, floxin, garenoxacin, gatifloxacin, gemifloxacin, grepafloxacin, kano trovafloxacin, levofloxacin, lomefloxacin, marbofloxacin, moxifloxacin, nadifloxacin, norfloxacin, orbifloxacin, ofloxacin, pefloxacin, trovafloxacin, grepafloxacin, sitafloxacin, sparfloxacin, temafloxacin, tosufloxacin, trovafloxacin; q). Streptogramins: pristinamycin, quinupristin/dalfopristin); r). Sulfonamides: mafenide, prontosil, sulfacetamide, sulfamethizole, sulfanilimide, sulfasalazine, sulfisoxazole, trimethoprim, trimethoprim-sulfamethoxazole (co-trimoxazole); s). Steroid antibacterials: e.g. fusidic acid; t). Tetracyclines: doxycycline, chlortetracycline, clomocycline, demeclocycline, lymecycline, meclocycline, metacycline, minocycline, oxytetracycline, penimepicycline, rolitetracycline, tetracycline, glycylcyclines (e.g. tigecycline); u). Other types of antibiotics: annonacin, arsphenamine, bactoprenol inhibitors (Bacitracin), DADAL/AR inhibitors (cycloserine), dictyostatin, discodermolide, eleutherobin, epothilone, ethambutol, etoposide, faropenem, fusidic acid, furazolidone, isoniazid, laulimalide, metronidazole, mupirocin, mycolactone, NAM synthesis inhibitors (e. g. fosfomycin), nitrofurantoin, paclitaxel, platensimycin, pyrazinamide, quinupristin/dalfopristin, rifampicin (rifampin), tazobactam tinidazole, uvaricin;

4). Anti-viral drugs: a). Entry/fusion inhibitors: aplaviroc, maraviroc, vicriviroc, gp41 (enfuvirtide), PRO 140, CD4 (ibalizumab); b). Integrase inhibitors: raltegravir, elvitegravir, globoidnan A; c). Maturation inhibitors: bevirimat, vivecon; d). Neuraminidase inhibitors: oseltamivir, zanamivir, peramivir; e). Nucleosides &nucleotides: abacavir, aciclovir, adefovir, amdoxovir, apricitabine, brivudine, cidofovir, clevudine, dexelvucitabine, didanosine (ddI), elvucitabine, emtricitabine (FTC), entecavir, famciclovir, fluorouracil (5-FU), 3′-fluoro-substituted 2′, 3′-dideoxynucleoside analogues (e.g. 3′-fluoro-2′,3′-dideoxythymidine (FLT) and 3′-fluoro-2′,3′-dideoxyguanosine (FLG), fomivirsen, ganciclovir, idoxuridine, lamivudine (3TC), 1-nucleosides (e.g. β-1-thymidine and β-1-2′-deoxycytidine), penciclovir, racivir, ribavirin, stampidine, stavudine (d4T), taribavirin (viramidine), telbivudine, tenofovir, trifluridine valaciclovir, valganciclovir, zalcitabine (ddC), zidovudine (AZT); f). Non-nucleosides: amantadine, ateviridine, capravirine, diarylpyrimidines (etravirine, rilpivirine), delavirdine, docosanol, emivirine, efavirenz, foscarnet (phosphonoformic acid), imiquimod, interferon alfa, loviride, lodenosine, methisazone, nevirapine, NOV-205, peginterferon alfa, podophyllotoxin, rifampicin, rimantadine, resiquimod (R-848), tromantadine; g). Protease inhibitors: amprenavir, atazanavir, boceprevir, darunavir, fosamprenavir, indinavir, lopinavir, nelfinavir, pleconaril, ritonavir, saquinavir, telaprevir (VX-950), tipranavir; h). Other types of anti-virus drugs: abzyme, arbidol, calanolide a, ceragenin, cyanovirin-n, diarylpyrimidines, epigallocatechin gallate (EGCG), foscarnet, griffithsin, taribavirin (viramidine), hydroxyurea, KP-1461, miltefosine, pleconaril, portmanteau inhibitors, ribavirin, seliciclib.

5). The drugs used for conjugates via a bis-linker of the present invention also include radioisotopes. Examples of radioisotopes (radionuclides) are ³H, ¹¹C, ¹⁴C, ¹⁸F, ³²P, ³⁵S, ⁶⁴Cu, ⁶⁸Ga, ⁸⁶Y, ⁹⁹T, ¹¹¹In, ¹²³I, ¹²⁴I, ¹²⁵I, ¹³¹I, ¹³³Xe, ¹⁷⁷Lu, ²¹¹At, or ²¹³Bi. Radioisotope labeled antibodies are useful in receptor targeted imaging experiments or can be for targeted treatment such as with the antibody-drug conjugates of the invention (Wu et al (2005) Nature Biotechnology 23(9): 1137-46). The cell binding molecules, e.g. an antibody can be labeled with ligand reagents through the bridge linkers of the present patent that bind, chelate or otherwise complex a radioisotope metal, using the techniques described in Current Protocols in Immunology, Volumes 1 and 2, Coligen et al, Ed. Wiley-Interscience, New York, Pubs. (1991). Chelating ligands which may complex a metal ion include DOTA, DOTP, DOTMA, DTPA and TETA (Macrocyclics, Dallas, Tex. USA).

6). The pharmaceutically acceptable salts, acids, derivatives, hydrate or hydrated salt; or a crystalline structure; or an optical isomer, racemate, diastereomer or enantiomer of any of the above drugs.

In another embodiment, the drug/cytotoxic molecule in the Formula (I) and/or (II) can be a chromophore molecule, for which the conjugate can be used for detection, monitoring, or study the interaction of the cell binding molecule with a target cell. Chromophore molecules are a compound that have the ability to absorb a kind of light, such as UV light, florescent light, IR light, near IR light, visual light; A chromatophore molecule includes a class or subclass of xanthophores, erythrophores, iridophores, leucophores, melanophores, and cyanophores; a class or subclass of fluorophore molecules which are fluorescent chemical compounds re-emitting light upon light; a class or subclass of visual phototransduction molecules; a class or subclass of photophore molecules; a class or subclass of luminescence molecules; and a class or subclass of luciferin compounds.

The chromophore molecule can be selected from, but not limited, non-protein organic fluorophores, such as: Xanthene derivatives (fluorescein, rhodamine, Oregon green, eosin, and Texas red); Cyanine derivatives: (cyanine, indocarbocyanine, oxacarbocyanine, thiacarbocyanine, and merocyanine); Squaraine derivatives and ring-substituted squaraines, including Seta, SeTau, and Square dyes; Naphthalene derivatives (dansyl and prodan derivatives); Coumarin derivatives; Oxadiazole derivatives (pyridyloxazole, nitrobenzoxadiazole and benzoxadiazole); Anthracene derivatives (anthraquinones, including DRAQ5, DRAQ7 and CyTRAK Orange); Pyrene derivatives (cascade blue, etc.); Oxazine derivatives (Nile red, Nile blue, cresyl violet, oxazine 170 etc.). Acridine derivatives (proflavin, acridine orange, acridine yellow etc.). Arylmethine derivatives (auramine, crystal violet, malachite green). Tetrapyrrole derivatives (porphin, phthalocyanine, bilirubin).

Or a chromophore molecule can be selected from any analogs and derivatives of the following fluorophore compounds: CF dye (Biotium), DRAQ and CyTRAK probes (BioStatus), BODIPY (Invitrogen), Alexa Fluor (Invitrogen), DyLight Fluor (Thermo Scientific, Pierce), Atto and Tracy (Sigma Aldrich), FluoProbes (Interchim), Abberior Dyes (Abberior), DY and MegaStokes Dyes (Dyomics), Sulfo Cy dyes (Cyandye), HiLyte Fluor (AnaSpec), Seta, SeTau and Square Dyes (SETA BioMedicals), Quasar and Cal Fluor dyes (Biosearch Technologies), SureLight Dyes (APC, RPEPerCP, Phycobilisomes)(Columbia Biosciences), APC, APCXL, RPE, BPE (Phyco-Biotech).

Examples of the widely used fluorophore compounds which are reactive or conjugatable with the linkers of the invention are: Allophycocyanin (APC), Aminocoumarin, APC-Cy7 conjugates, BODIPY-FL, Cascade Blue, Cy2, Cy3, Cy3.5, Cy3B, Cy5, Cy5.5, Cy7, Fluorescein, FluorX, Hydroxycoumarin, IR-783, Lissamine Rhodamine B, Lucifer yellow, Methoxycoumarin, NBD, Pacific Blue, Pacific Orange, PE-Cy5 conjugates, PE-Cy7 conjugates, PerCP, R-Phycoerythrin (PE), Red 613, Seta-555-Azide, Seta-555-DBCO, Seta-555-NHS, Seta-580-NHS, Seta-680-NHS, Seta-780-NHS, Seta-APC-780, Seta-PerCP-680, Seta-R-PE-670, SeTau-380-NHS, SeTau-405-Maleimide, SeTau-405-NHS, SeTau-425-NHS, SeTau-647-NHS, Texas Red, TRITC, TruRed, X-Rhodamine.

The fluorophore compounds that can be linked to the linkers of the invention for study of nucleic acids or proteins are selected from the following compounds or their derivatives: 7-AAD (7-aminoactinomycin D, CG-selective), Acridine Orange, Chromomycin A3, CyTRAK Orange (Biostatus, red excitation dark), DAPI, DRAQ5, DRAQ7, Ethidium Bromide, Hoechst33258, Hoechst33342, LDS 751, Mithramycin, Propidiumlodide (PI), SYTOX Blue, SYTOX Green, SYTOX Orange, Thiazole Orange, TO-PRO: Cyanine Monomer, TOTO-1, TO-PRO-1, TOTO-3, TO-PRO-3, YOSeta-1, YOYO-1. The fluorophore compounds that can be linked to the linkers of the invention for study cells are selected from the following compounds or their derivatives: DCFH (2′7′Dichorodihydro-fluorescein, oxidized form), DHR (Dihydrorhodamine 123, oxidized form, light catalyzes oxidation), Fluo-3 (AM ester. pH>6), Fluo-4 (AM ester. pH 7.2), Indo-1 (AM ester, low/high calcium (Ca2+)), and SNARF (pH 6/9). The preferred fluorophore compounds that can be linked to the linkers of the invention for study proteins/antibodies are selected from the following compounds or their derivatives: Allophycocyanin (APC), AmCyan1 (tetramer, Clontech), AsRed2 (tetramer, Clontech), Azami Green (monomer, MBL), Azurite, B-phycoerythrin (BPE), Cerulean, CyPet, DsRed monomer (Clontech), DsRed2 (“RFP”, Clontech), EBFP, EBFP2, ECFP, EGFP (weak dimer, Clontech), Emerald (weak dimer, Invitrogen), EYFP (weak dimer, Clontech), GFP (S65A mutation), GFP (S65C mutation), GFP (S65L mutation), GFP (S65T mutation), GFP (Y66F mutation), GFP (Y66H mutation), GFP (Y66W mutation), GFPuv, HcRed1, J-Red, Katusha, Kusabira Orange (monomer, MBL), mCFP, mCherry, mCitrine, Midoriishi Cyan (dimer, MBL), mKate (TagFP635, monomer, Evrogen), mKeima-Red (monomer, MBL), mKO, mOrange, mPlum, mRaspberry, mRFP1 (monomer, Tsien lab), mStrawberry, mTFP1, mTurquoise2, P3 (phycobilisome complex), Peridinin Chlorophyll (PerCP), R-phycoerythrin (RPE), T-Sapphire, TagCFP (dimer, Evrogen), TagGFP (dimer, Evrogen), TagRFP (dimer, Evrogen), TagYFP (dimer, Evrogen), tdTomato (tandem dimer), Topaz, TurboFP602 (dimer, Evrogen), TurboFP635 (dimer, Evrogen), TurboGFP (dimer, Evrogen), TurboRFP (dimer, Evrogen), TurboYFP (dimer, Evrogen), Venus, Wild Type GFP, YPet, ZsGreen1 (tetramer, Clontech), ZsYellow1 (tetramer, Clontech).

The examples of the structure of the conjugates of the antibody-chromophore molecules via the bridge linker are as following Ac01, Ac02, Ac03, Ac04, Ac05, Ac06, and Ac07:

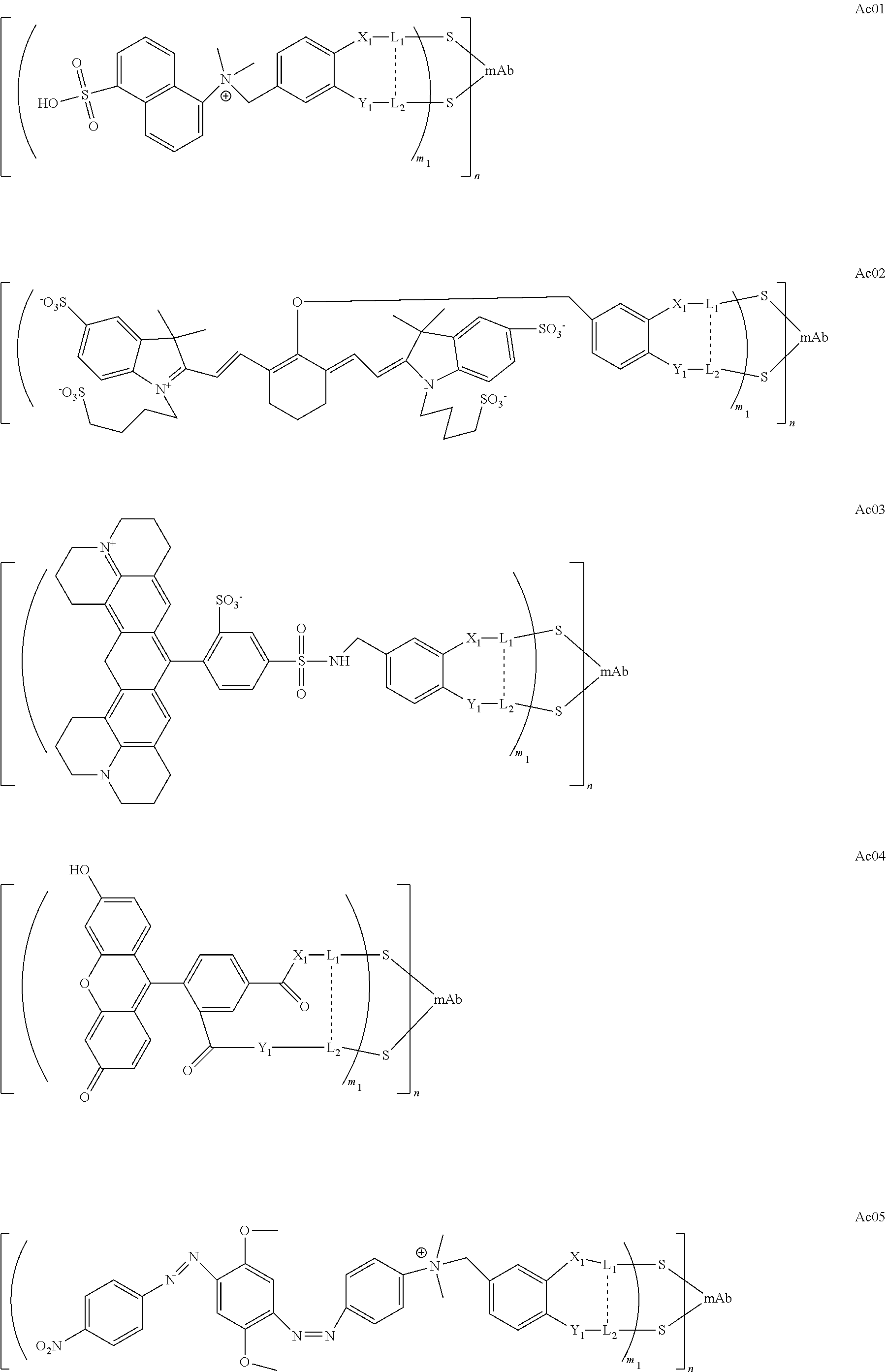

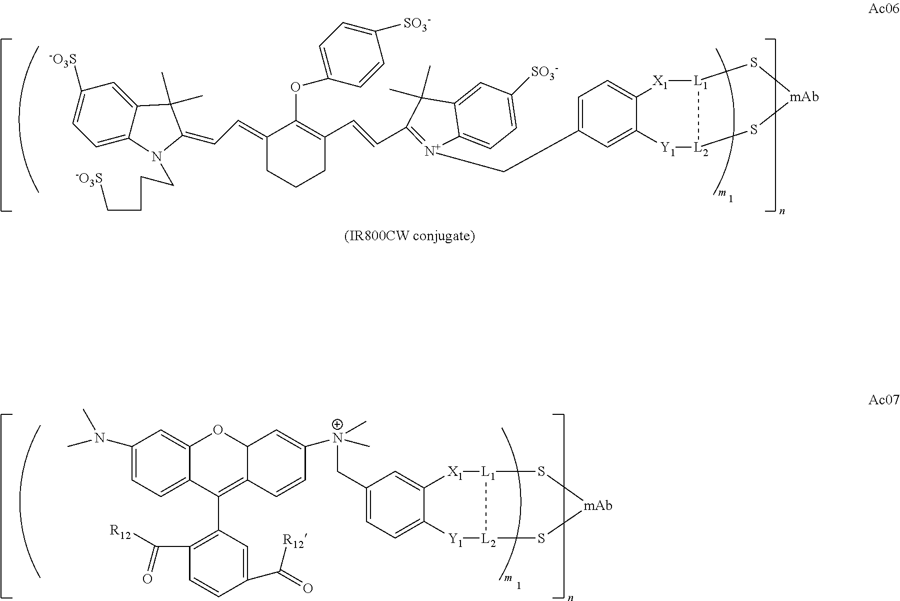

Wherein “-----” is optionally either a single bond, or a double bond, or can optionally be absent; X₁, and Y₁ are independently O, NH, NHNH, NR₅, S, C(O)O, C(O)NH, OC(O)NH, OC(O)O, NHC(O)NH, NHC(O)S, OC(O)N(R₁), N(R₁)C(O)N(R₁), CH, C(O)NHNHC(O) and C(O)NR₁; mAb is antibody, preferably monoclonal antibody; n and m₁ are independently 1-20; R₁₂ and R₁₂′ are independently OH, NH₂, NHR₁, NHNH₂, NHNHCOOH, O—R₁—COOH, NH—R₁—COOH, NH-(Aa)ₙCOOH, O(CH₂CH₂O)ₚCH₂CH₂OH, O(CH₂CH₂O)ₚCH₂CH₂NH₂, NH(CH₂CH₂O)ₚCH₂CH₂NH₂, O(CH₂CH₂O)ₚCH₂CH₂COOH, NH(CH₂CH₂O)ₚCH₂CH₂COOH, O(CH₂CH₂O)ₚCH₂CH₂NHSO₃H, NH(CH₂CH₂O)ₚCH₂CH₂NHSO₃H, R₁—NHSO₃H, NH—R₁—NHSO₃H, O(CH₂CH₂O)ₚCH₂CH₂NHPO₃H₂, NH(CH₂CH₂O)ₚCH₂CH₂NHPO₃H₂, R₁—NHPO₃H₂, R₁—OPO₃H₂, O(CH₂CH₂O)ₚCH₂CH₂OPO₃H₂, NH(CH₂CH₂O)ₚCH₂CH₂NHPO₃H₂, OR₁—NHPO₃H₂, NH—R₁—NHPO₃H₂, NH—Ar—COOH, NH—Ar—NH₂, wherein p=0-5000, Aa is an aminoacid; R₁, m₁, n, L₁, and L₂ are the same defined in Formula (I).

In another embodiment, the drug in the Formula (I) and (II) can be polyalkylene glycols that are used for extending the half-life of the cell-binding molecule when administered to a mammal. Polyalkylene glycols include, but are not limited to, poly(ethylene glycols) (PEGs), poly(propylene glycol) and copolymers of ethylene oxide and propylene oxide; particularly preferred are PEGs, and more particularly preferred are monofunctionally activated hydroxyPEGs (e.g., hydroxyl PEGs activated at a single terminus, including reactive esters of hydroxyPEG-monocarboxylic acids, hydroxyPEG-monoaldehydes, hydroxyPEG-monoamines, hydroxyPEG-monohydrazides, hydroxyPEG-monocarbazates, hydroxyl PEG-monoiodoacetamides, hydroxyl PEG-monomaleimides, hydroxyl PEG-monoorthopyridyl disulfides, hydroxyPEG-monooximes, hydroxyPEG-monophenyl carbonates, hydroxyl PEG-monophenyl glyoxals, hydroxyl PEG-monothiazolidine-2-thiones, hydroxyl PEG-monothioesters, hydroxyl PEG-monothiols, hydroxyl PEG-monotriazines and hydroxyl PEG-monovinylsulfones).

In certain such embodiments, the polyalkylene glycol has a molecular weight of from about 10 Daltons to about 200 kDa, preferably about 88 Da to about 40 kDa; two branches each with a molecular weight of about 88 Da to about 40 kDa; and more preferably two branches, each of about 88 Da to about 20 kDa. In one particular embodiment, the polyalkylene glycol is poly(ethylene) glycol and has a molecular weight of about 10 kDa; about 20 kDa, or about 40 kDa. In specific embodiments, the PEG is a PEG 10 kDa (linear or branched), a PEG 20 kDa (linear or branched), or a PEG 40 kDa (linear or branched). A number of US patents have disclosed the preparation of linear or branched “non-antigenic” PEG polymers and derivatives or conjugates thereof, e.g., U.S. Pat. Nos. 5,428,128; 5,621,039; 5,622,986; 5,643,575; 5,728,560; 5,730,990; 5,738,846; 5,811,076; 5,824,701; 5,840,900; 5,880,131; 5,900,402; 5,902,588; 5,919,455; 5,951,974; 5,965,119; 5,965,566; 5,969,040; 5,981,709; 6,011,042; 6,042,822; 6,113,906; 6,127,355; 6,132,713; 6,177,087, and 6,180,095. The structure of the conjugates of the antibody-polyalkylene glycols via the bridge linker is as following Pg01, Pg02, and Pg03.

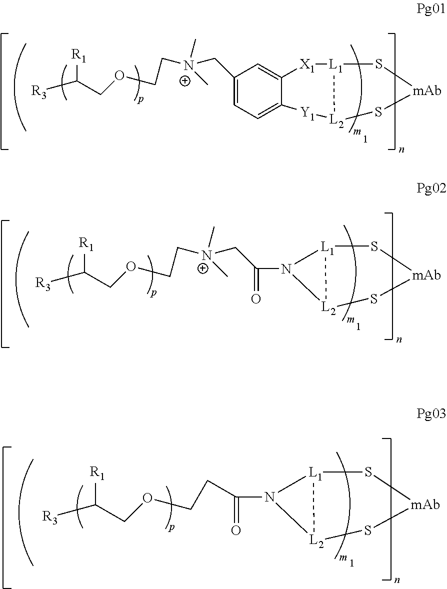

wherein “-----” is optionally either a single bond, or a double bond, or can optionally be absent; X₁, and Y₁ are independently O, NH, NHNH, NR₅, S, C(O)O, C(O)NH, OC(O)NH, OC(O)O, NHC(O)NH, NHC(O)S, OC(O)N(R₁), N(R₁)C(O)N(R₁), CH, C(O)NHNHC(O) and C(O)NR₁; mAb is antibody, preferably monoclonal antibody; n and m₁ are independently 1-20; p is 1-5000; R₁, L₁, and L₂ are the same defined in Formula (I). Preferably R₁ and R₃ is H, OH, OCH₃, CH₃, or OC₂H₅ independently.

In yet another embodiment, the preferred cytotoxic agents that conjugated to a cell-binding molecule via a bridge linker of this patent are tubulysins, maytansinoids, taxanoids (taxanes), CC-1065 analogs, daunorubicin and doxorubicin compounds, amatoxins (including amanitins), indolecarboxamide, benzodiazepine dimers (e.g., dimers of pyrrolobenzodiazepine (PBD), tomaymycin, anthramycin, indolinobenzodiazepines, imidazobenzothiadiazepines, or oxazolidinobenzodiazepines), calicheamicins and the enediyne antibiotics, actinomycin, azaserines, bleomycins, epirubicin, eribulin, tamoxifen, idarubicin, dolastatins, auristatins (e.g. monomethyl auristatin E, MMAE, MMAF, auristatin PYE, auristatin TP, Auristatins 2-AQ, 6-AQ, EB (AEB), and EFP (AEFP) and their analogs), duocarmycins, geldanamycins or other HSP90 inhibitors, centanamycin, methotrexates, thiotepa, vindesines, vincristines, hemiasterlins, nazumamides, microginins, radiosumins, streptonigtin, SN38 or other analogs or metabolites of camptothecin, alterobactins, microsclerodermins, theonellamides, esperamicins, PNU-159682; and their analogues or derivatives, pharmaceutically acceptable salts, acids, derivatives, hydrate or hydrated salt; or a crystalline structure; or an optical isomer, racemate, diastereomer or enantiomer of any of the above drugs thereof.

Tubulysins that are preferred for conjugation in the present invention are well known in the art and can be isolated from natural sources according to known methods or prepared synthetically according to known methods (e. g. Balasubramanian, R., et al. J. Med. Chem., 2009, 52, 238-40; Wipf, P., et al. Org. Lett., 2004, 6, 4057-60; Pando, O., et al. J. Am. Chem. Soc., 2011, 133, 7692-5; Reddy, J. A., et al. Mol. Pharmaceutics, 2009, 6, 1518-25; Raghavan, B., et al. J. Med. Chem., 2008, 51, 1530-33; Patterson, A. W., et al. J. Org. Chem., 2008, 73, 4362-9; Pando, O., et al. Org. Lett., 2009, 11 (24), 5567-9; Wipf, P., et al. Org. Lett., 2007, 9 (8), 1605-7; Friestad, G. K., Org. Lett., 2004, 6, 3249-52; Peltier, H. M., et al. J. Am. Chem. Soc., 2006, 128, 16018-9; Chandrasekhar, S., et al J. Org. Chem., 2009, 74, 9531-4; Liu, Y., et al. Mol. Pharmaceutics, 2012, 9, 168-75; Friestad, G. K., et al. Org. Lett., 2009, 11, 1095-8; Kubicek, K., et al., Angew Chem Int Ed Engl, 2010.49: 4809-12; Chai, Y., et al., Chem Biol, 2010, 17: 296-309; Ullrich, A., et al., Angew Chem Int Ed Engl, 2009, 48, 4422-5; Sani, M., et al. Angew Chem Int Ed Engl, 2007, 46, 3526-9; Domling, A., et al., Angew Chem Int Ed Engl, 2006, 45, 7235-9; patent applications: Zanda, M., et al, Can. Pat. Appl. CA 2710693 (2011); Chai, Y., et al. Eur. Pat. Appl. 2174947 (2010), WO 2010034724; Leamon, C. et al, WO2010033733, WO 2009002993; Ellman, J., et al, PCT WO2009134279; WO 2009012958, US appl. 20110263650, 20110021568; Matschiner, G., et al, WO2009095447; Vlahov, I., et al, WO2009055562, WO 2008112873; Low, P., et al, WO2009026177; Richter, W., WO2008138561; Kjems, J., et al, WO 2008125116; Davis, M.; et al, WO2008076333; Diener, J.; et al, U.S. Pat. Appl. 20070041901, WO2006096754; Matschiner, G., et al, WO2006056464; Vaghefi, F., et al, WO2006033913; Doemling, A., Ger. Offen. DE102004030227, WO2004005327, WO2004005326, WO2004005269; Stanton, M., et al, U.S. Pat. Appl. Publ. 20040249130; Hoefle, G., et al, Ger. Offen. DE10254439, DE10241152, DE10008089; Leung, D., et al, WO2002077036; Reichenbach, H., et al, Ger. Offen. DE19638870; Wolfgang, R., US20120129779; Chen, H., US appl. 20110027274. The preferred structures of tubulysins for conjugation of cell binding molecules are described in the patent application of PCT/IB2012/053554.

Examples of the structures of the conjugates of the antibody-tubulysin analogs via a bis-linker are T01, T02, T03, T04, T05, T06 T07, T08, T09, T10 and T11 as following:

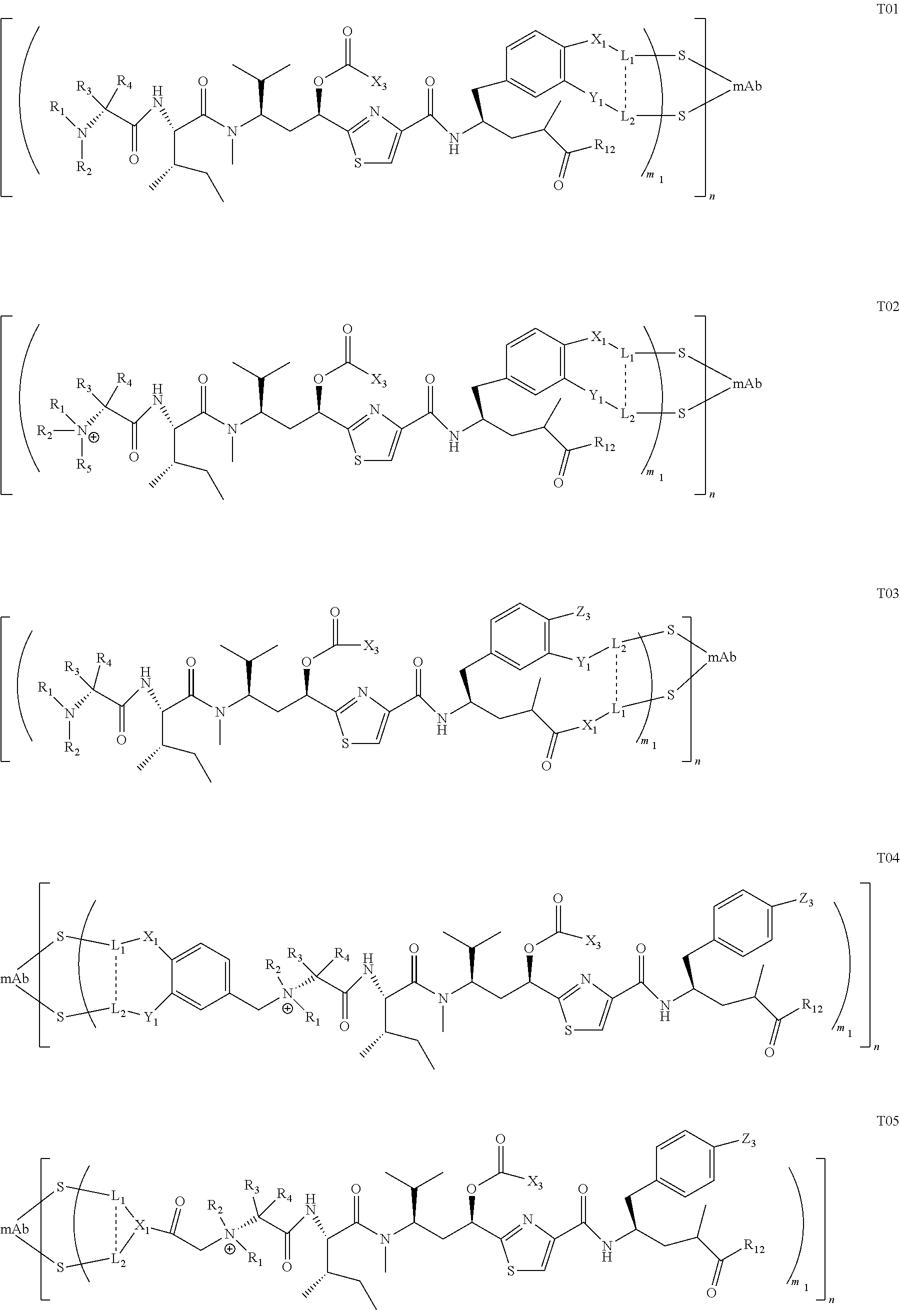

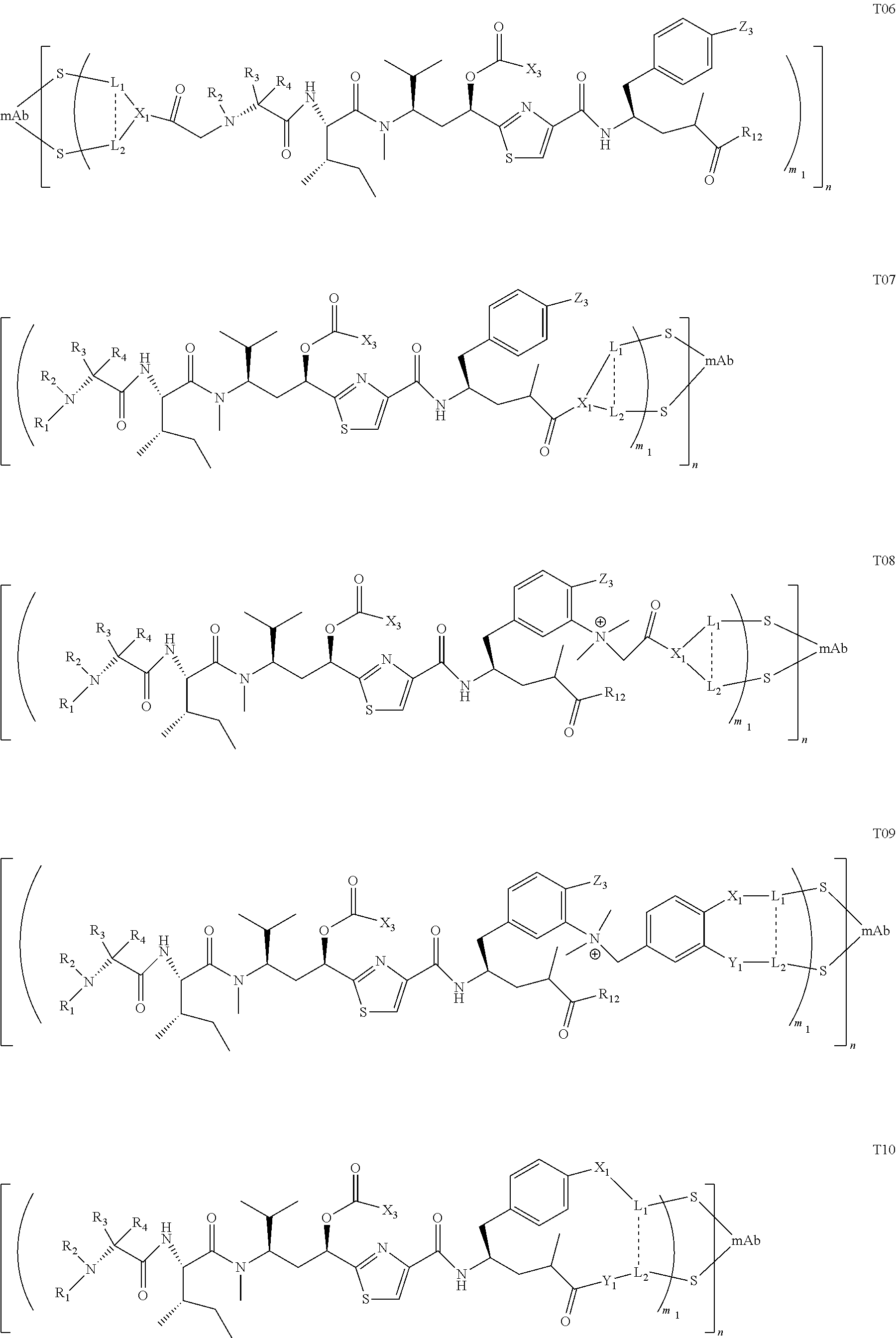

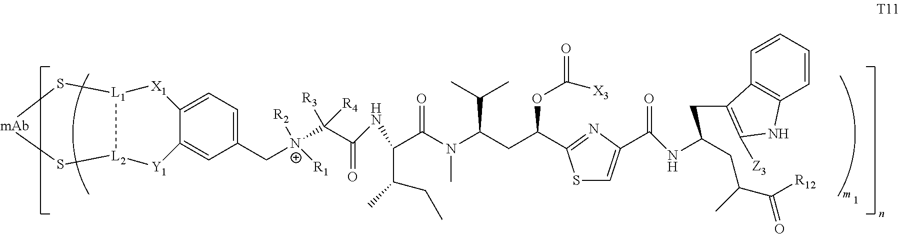

wherein “-----” is optionally either a single bond, or a double bond, or can optionally be absent; X₁, and Y₁ are independently O, NH, NHNH, NR₅, S, C(O)O, C(O)NH, OC(O)NH, OC(O)O, NHC(O)NH, NHC(O)S, OC(O)N(R₁), N(R₁)C(O)N(R₁), CH, C(O)NHNHC(O) and C(O)NR₁; mAb is antibody, preferably monoclonal antibody; R₁₂ is OH, NH₂, NHR₁, NHNH₂, NHNHCOOH, O—R₁—COOH, NH—R₁—COOH, NH-(Aa)ₙCOOH, O(CH₂CH₂O)ₚCH₂CH₂OH, O(CH₂CH₂O)ₚCH₂CH₂NH₂, NH(CH₂CH₂O)ₚCH₂CH₂NH₂, NR₁R₁′, NHOH, NHOR₁, O(CH₂CH₂O)ₚCH₂CH₂COOH, NH(CH₂CH₂O)ₚCH₂CH₂COOH, NH—Ar—COOH, NH—Ar—NH₂, O(CH₂CH₂O)ₚCH₂CH₂NHSO₃H, NH(CH₂CH₂O)ₚCH₂CH₂NHSO₃H, R₁—NHSO₃H, NH—R₁—NHSO₃H, O(CH₂CH₂O)ₚCH₂CH₂NHPO₃H₂, NH(CH₂CH₂O)ₚCH₂CH₂NHPO₃H₂, OR₁, R₁—NHPO₃H₂, R₁—OPO₃H₂, O(CH₂CH₂O)ₚCH₂CH₂OPO₃H₂, OR₁—NHPO₃H₂, NH—R₁—NHPO₃H₂, NH(CH₂CH₂NH)ₚCH₂CH₂NH₂, NH(CH₂CH₂S)ₚCH₂CH₂NH₂, NH(CH₂CH₂NH)ₚCH₂CH₂OH, NH(CH₂CH₂S)ₚCH₂CH₂OH, NH—R₁—NH₂, or NH(CH₂CH₂O)ₚCH₂CH₂NHPO₃H₂, wherein Aa is 1-8 aminoacids; n and m₁ are independently 1-20; p is 1-5000; Preferably R₁, R₁′, R₂, R₃, and R₄ are independently H, C₁-C₈ lineal or branched alkyl, amide, or amines; C₂-C₈ aryl, alkenyl, alkynyl, heteroaryl, heteroalkyl, alkylcycloalkyl, ester, ether, heterocycloalkyl, or acyloxylamines; or peptides containing 1-8 aminoacids, or polyethyleneoxy unit having formula (OCH₂CH₂)ₚ or (OCH₂CH(CH₃))ₚ, wherein p is an integer from 1 to about 5000; The two Rs: R₁R₂, R₂R₃, R₁R₃ or R₃R₄ can form 3˜8 member cyclic ring of alkyl, aryl, heteroaryl, heteroalkyl, or alkylcycloalkyl group; X₃ is H, CH₃, CH₂CH₃, C₃H7, or X₁′R₁′, wherein X₁′ is NH, N(CH₃), NHNH, O, or S; R₁′ is H or C₁-C₈ lineal or branched alkyl, aryl, heteroaryl, heteroalkyl, alkylcycloalkyl, or acyloxylamines; R₃′ is H or C₁-C₆ lineal or branched alkyl; Z₃ is H, COOR₁, NH₂, NHR₁, OR₁, CONHR₁, NHCOR₁, OCOR₁, OP(O)(OM₁)(OM₂), OCH₂OP(O)(OM₁)(OM₂), OSO₃M₁, R₁, O-glycoside (glucoside, galactoside, mannoside, glucuronoside/glucuronide, alloside, fructoside, etc.), NH-glycoside, S-glycoside or CH₂-glycoside; M₁ and M₂ are independently H, Na, K, Ca, Mg, NH₄, NR₁R₂R₃; L₁, and L₂ are defined the same in Formula (I).

Calicheamicins and their related enediyne antibiotics that are preferred for cell-binding molecule-drug conjugates of this patent are described in: Nicolaou, K. C. et al, Science 1992, 256, 1172-1178; Proc. Natl. Acad. Sci USA. 1993, 90, 5881-8), U.S. Pat. Nos. 4,970,198; 5,053,394; 5,108,912; 5,264,586; 5,384,412; 5,606,040; 5,712,374; 5,714,586; 5,739,116; 5,770,701; 5,770,710; 5,773,001; 5,877,296; 6,015,562; 6,124,310; 8,153,768. Examples of the structure of the conjugate of the antibody-Calicheamicin analog via the bridge linker are C01 and C02 as the following:

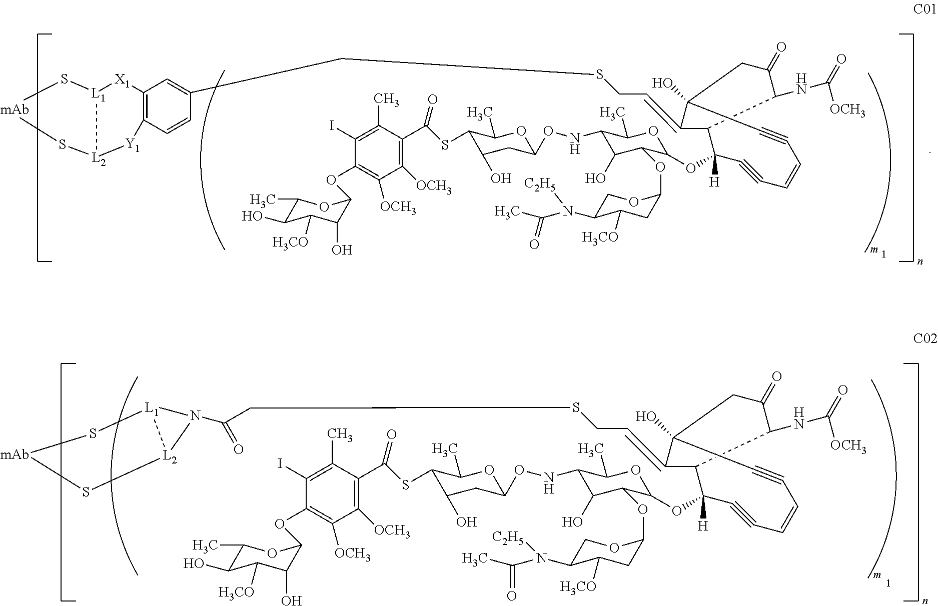

wherein “-----” is optionally either a single bond, or a double bond, or can optionally be absent; X₁ and Y₁ are independently O, NH, NHNH, NR₅, S, C(O)O, C(O)NH, OC(O)NH, OC(O)O, NHC(O)NH, NHC(O)S, OC(O)N(R₁), N(R₁)C(O)N(R₁), CH, C(O)NHNHC(O) and C(O)NR₁; mAb is antibody, preferably monoclonal antibody; n and m₁ are independently 1-20; p is 1-5000; R₁, L₁, and L₂ are the same defined in Formula (I).

Maytansinoids that are preferred to be used in the present invention including maytansinol and its analogues are described in U.S. Pat. Nos. 4,256,746, 4,361,650, 4,307,016, 4,294,757, 4,294,757, 4,371,533, 4,424,219, 4,331,598, 4,450,254, 4,364,866, 4,313,946, 4,315,929 4,362,663, 4,322,348, 4,371,533, 4,424,219, 5,208,020, 5,416,064, 5,208,020; 5,416,064; 6,333,410; 6,441,163; 6,716,821, 7,276,497, 7,301,019, 7,303,749, 7,368,565, 7,411,063, 7,851,432, and 8,163,888. An example of the structure of the conjugate of the antibody-Maytansinoids via the linker of the patent is as the following My01, My02, My03, My04, My05, and My06:

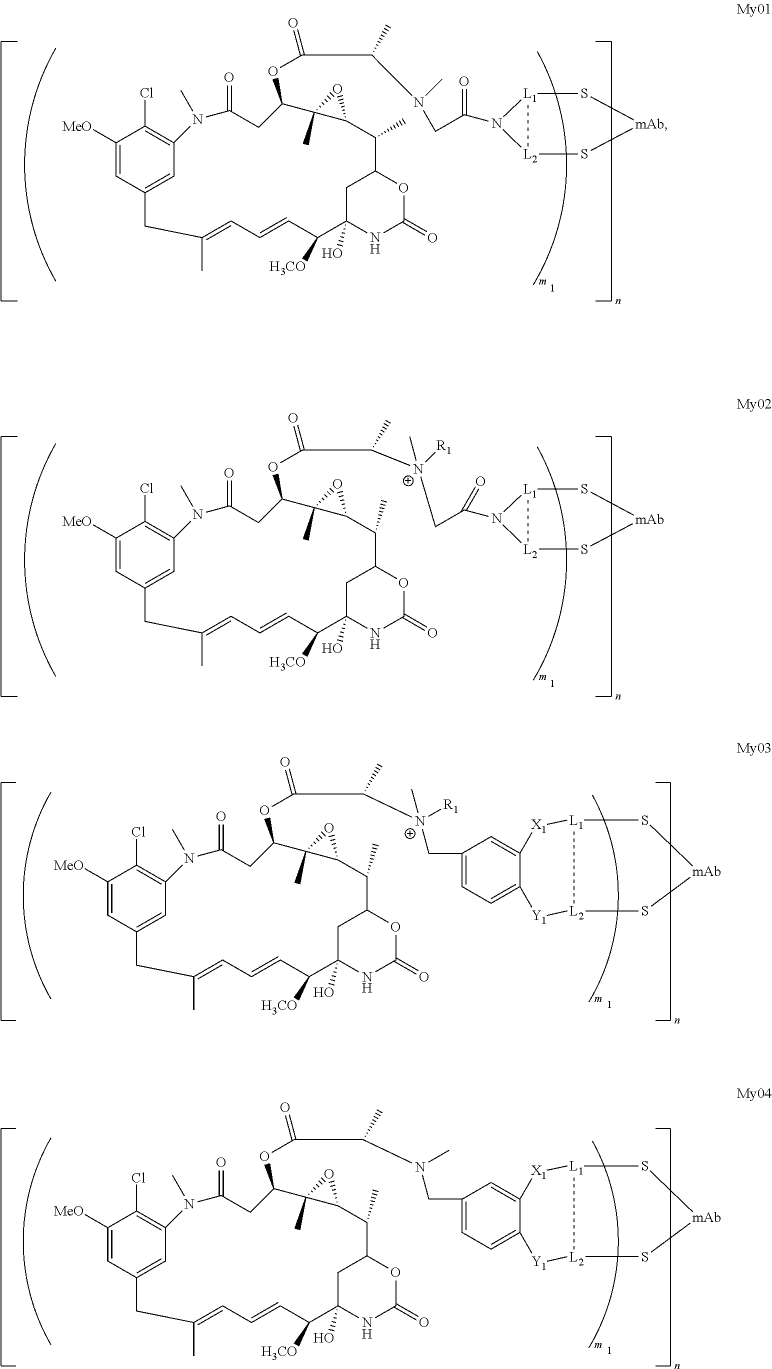

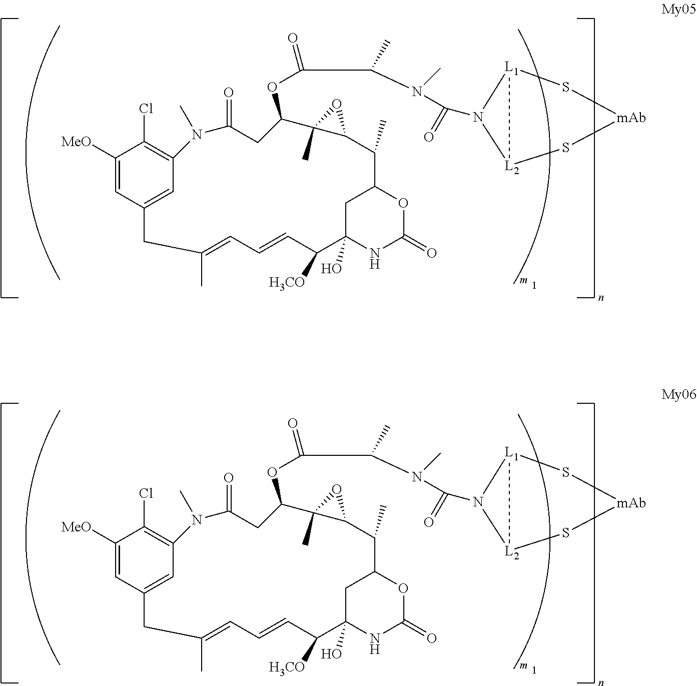

wherein “-----” is optionally either a single bond, or a double bond, or can optionally be absent; X₁, and Y₁ are independently O, NH, NHNH, NR₅, S, C(O)O, C(O)NH, OC(O)NH, OC(O)O, NHC(O)NH, NHC(O)S, OC(O)N(R₁), N(R₁)C(O)N(R₁), CH, C(O)NHNHC(O) and C(O)NR₁; mAb is antibody, preferably monoclonal antibody; n and m₁ are independently 1-20; p is 1-5000; R₁, L₁, and L₂ are the same defined in Formula (I).

Taxanes, which includes Paclitaxel (Taxol), a cytotoxic natural product, and docetaxel (Taxotere), a semi-synthetic derivative, and their analogs which are preferred for conjugation are exampled in: K C. Nicolaou et al., J. Am. Chem. Soc. 117, 2409-20, (1995); Ojima et al, J. Med. Chem. 39:3889-3896 (1996); 40:267-78 (1997); 45, 5620-3 (2002); Ojima et al., Proc. Natl. Acad. Sci., 96:4256-61 (1999); Kim et al., Bull. Korean Chem. Soc., 20, 1389-90 (1999); Miller, et al. J. Med. Chem., 47, 4802-5 (2004); U.S. Pat. No. 5,475,011 5,728,849, 5,811,452; 6,340,701; 6,372,738; 6,391,913, 6,436,931; 6,589,979; 6,596,757; 6,706,708; 7,008,942; 7,186,851; 7,217,819; 7,276,499; 7,598,290; and 7,667,054.

Examples of the structures of the conjugate of the antibody-taxanes via the linker of the patent are as the following Tx01, Tx02 and Tx03.

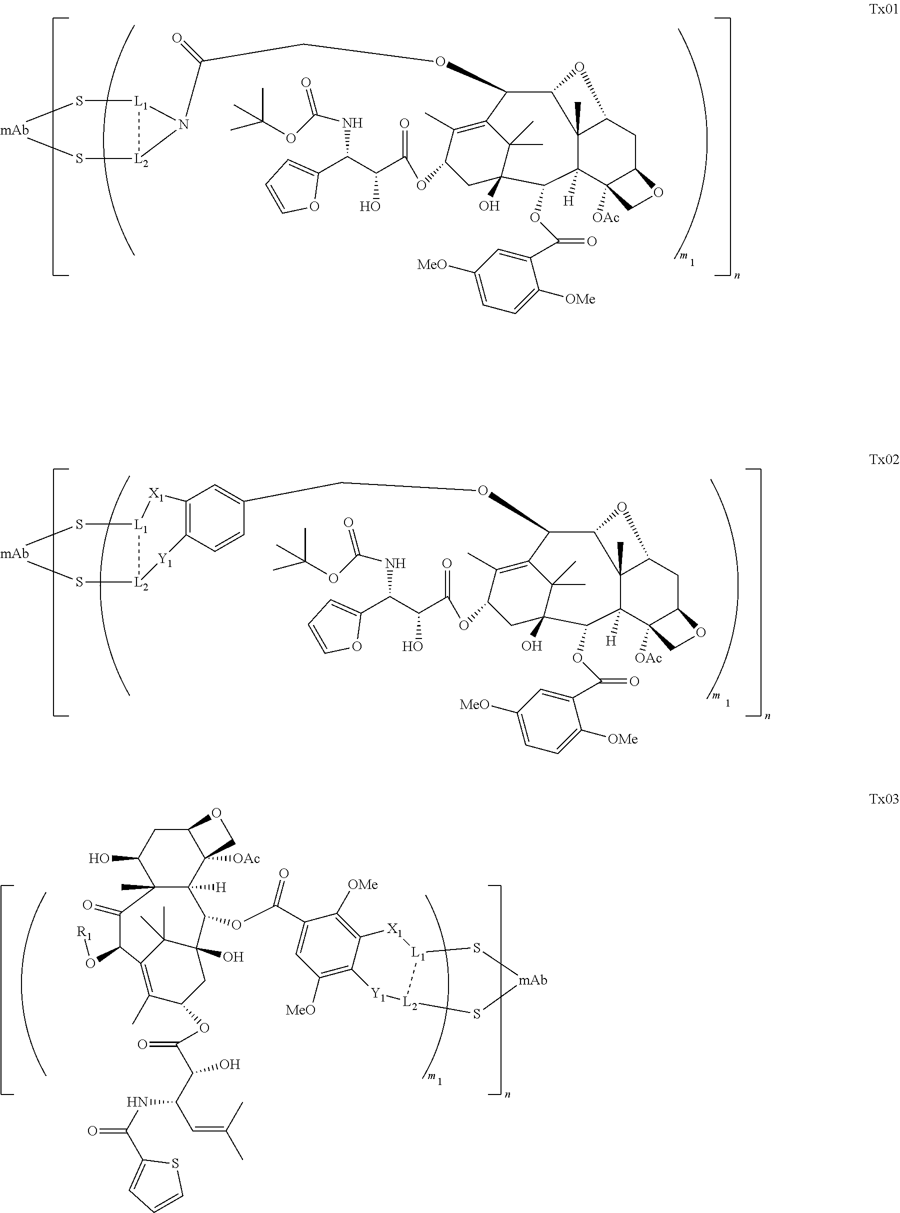

wherein “-----” is optionally either a single bond, or a double bond, or can optionally be absent; X₁, and Y₁ are independently O, NH, NHNH, NR₅, S, C(O)O, C(O)NH, OC(O)NH, OC(O)O, NHC(O)NH, NHC(O)S, OC(O)N(R₁), N(R₁)C(O)N(R₁), CH, C(O)NHNHC(O) and C(O)NR₁; mAb is antibody, preferably monoclonal antibody; n and m₁ are independently 1-20; R₁, L₁, and L₂ are the same defined in Formula (I).

CC-1065 analogues and doucarmycin analogs are also preferred to be used for a conjugate containing bis-bridge linkage of the present patent. The examples of the CC-1065 analogues and doucarmycin analogs as well as their synthesis are described in: e.g. Warpehoski, et al, J. Med. Chem. 31:590-603 (1988); D. Boger et al., J. Org. Chem; 66; 6654-61, 2001; U.S. Pat. Nos. 4,169,888, 4,391,904, 4,671,958, 4,816,567, 4,912,227, 4,923,990, 4,952,394, 4,975,278, 4,978,757, 4,994,578, 5,037,993, 5,070,092, 5,084,468, 5,101,038, 5,117,006, 5,137,877, 5,138,059, 5,147,786, 5,187,186, 5,223,409, 5,225,539, 5,288,514, 5,324,483, 5,332,740, 5,332,837, 5,334,528, 5,403,484, 5,427,908, 5,475,092, 5,495,009, 5,530,101, 5,545,806, 5,547,667, 5,569,825, 5,571,698, 5,573,922, 5,580,717, 5,585,089, 5,585,499, 5,587,161, 5,595,499, 5,606,017, 5,622,929, 5,625,126, 5,629,430, 5,633,425, 5,641,780, 5,660,829, 5,661,016, 5,686,237, 5,693,762, 5,703,080, 5,712,374, 5,714,586, 5,739,116, 5,739,350, 5,770,429, 5,773,001, 5,773,435, 5,786,377 5,786,486, 5,789,650, 5,814,318, 5,846,545, 5,874,299, 5,877,296, 5,877,397, 5,885,793, 5,939,598, 5,962,216, 5,969,108, 5,985,908, 6,060,608, 6,066,742, 6,075,181, 6,103,236, 6,114,598, 6,130,237, 6,132,722, 6,143,901, 6,150,584, 6,162,963, 6,172,197, 6,180,370, 6,194,612, 6,214,345, 6,262,271, 6,281,354, 6,310,209, 6,329,497, 6,342,480, 6,486,326, 6,512,101, 6,521,404, 6,534,660, 6,544,731, 6,548,530, 6,555,313, 6,555,693, 6,566,336, 6,586,618, 6,593,081, 6,630,579, 6,756,397, 6,759,509, 6,762,179, 6,884,869, 6,897,034, 6,946,455, 7,049,316, 7,087,600, 7,091,186, 7,115,573, 7,129,261, 7,214,663, 7,223,837, 7,304,032, 7,329,507, 7,329,760, 7,388,026, 7,655,660, 7,655,661, 7,906,545, and 8,012,978. Examples of the structures of the conjugate of the antibody-CC-1065 analogs via the linker of the patent are as the following CC01, CC02, CC03 and CC04.

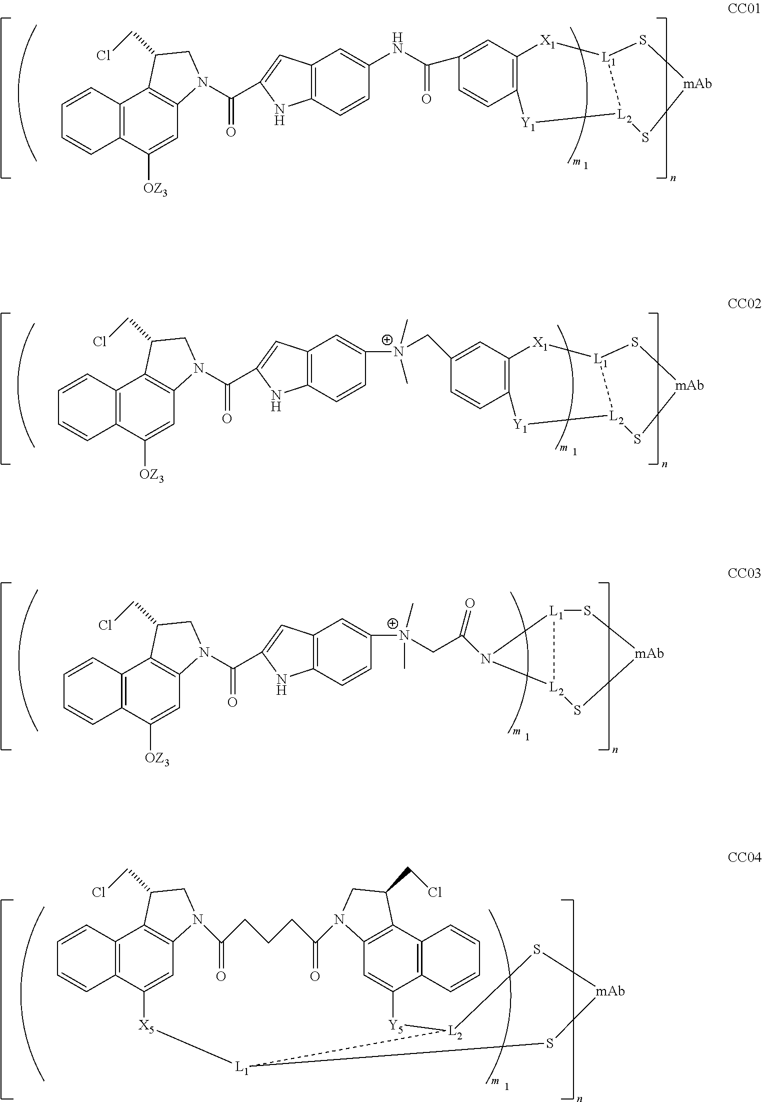

Wherein mAb is an antibody; Z₃ is H, PO(OM₁)(OM₂), SO₃M₁, CH₂PO(OM₁)(OM₂), CH₃N(CH₂CH₂)₂NC(O)—, O(CH₂CH₂)₂NC(O)—, R₁, or glycoside; wherein “-----” is optionally either a single bond, or a double bond, or can optionally be absent; X₁, X₅, Y₁ and Y₅ are independently O, NH, NHNH, NR₅, S, C(O)O, C(O)NH, OC(O)NH, OC(O)O, NHC(O)NH, NHC(O)S, OC(O)N(R₁), N(R₁)C(O)N(R₁), CH, C(O)NHNHC(O) and C(O)NR₁; mAb is antibody, preferably monoclonal antibody; n and m₁ are independently 1-20; R₁, L₁, and L₂ are the same defined in Formula (I).

Daunorubicin/Doxorubicin Analogues are also preferred for conjugation having the bis-linkage of the present patent. The preferred structures and their synthesis are exampled in: Hurwitz, E., et al., Cancer Res. 35, 1175-81 (1975). Yang, H. M., and Reisfeld, R. A., Proc. Natl. Acad. Sci. 85, 1189-93 (1988); Pietersz, C. A., E., et al., E., et al.,” Cancer Res. 48, 926-311 (1988); Trouet, et al., 79, 626-29 (1982); Z. Brich et al., J. Controlled Release, 19, 245-58 (1992); Chen et al., Syn. Comm., 33, 2377-90, 2003; King et al., Bioconj. Chem., 10, 279-88, 1999; King et al., J. Med. Chem., 45, 4336-43, 2002; Kratz et al., J Med Chem. 45, 5523-33, 2002; Kratz et al., Biol Pharm Bull. January 21, 56-61, 1998; Lau et al., Bioorg. Med. Chem. 3, 1305-12, 1995; Scott et al., Bioorg. Med. Chem. Lett. 6, 1491-6, 1996; Watanabe et al., Tokai J. Experimental Clin. Med. 15, 327-34, 1990; Zhou et al., J. Am. Chem. Soc. 126, 15656-7, 2004; WO 01/38318; U.S. Pat. Nos. 5,106,951; 5,122,368; 5,146,064; 5,177,016; 5,208,323; 5,824,805; 6,146,658; 6,214,345; 7,569,358; 7,803,903; 8,084,586; 8,053,205. Examples of the structures of the conjugate of the antibody-CC-1065 analogs via the linker of the patent are as the following Da01, Da02, Da03, Da04, Da05, Da06, Da07 and Da08.

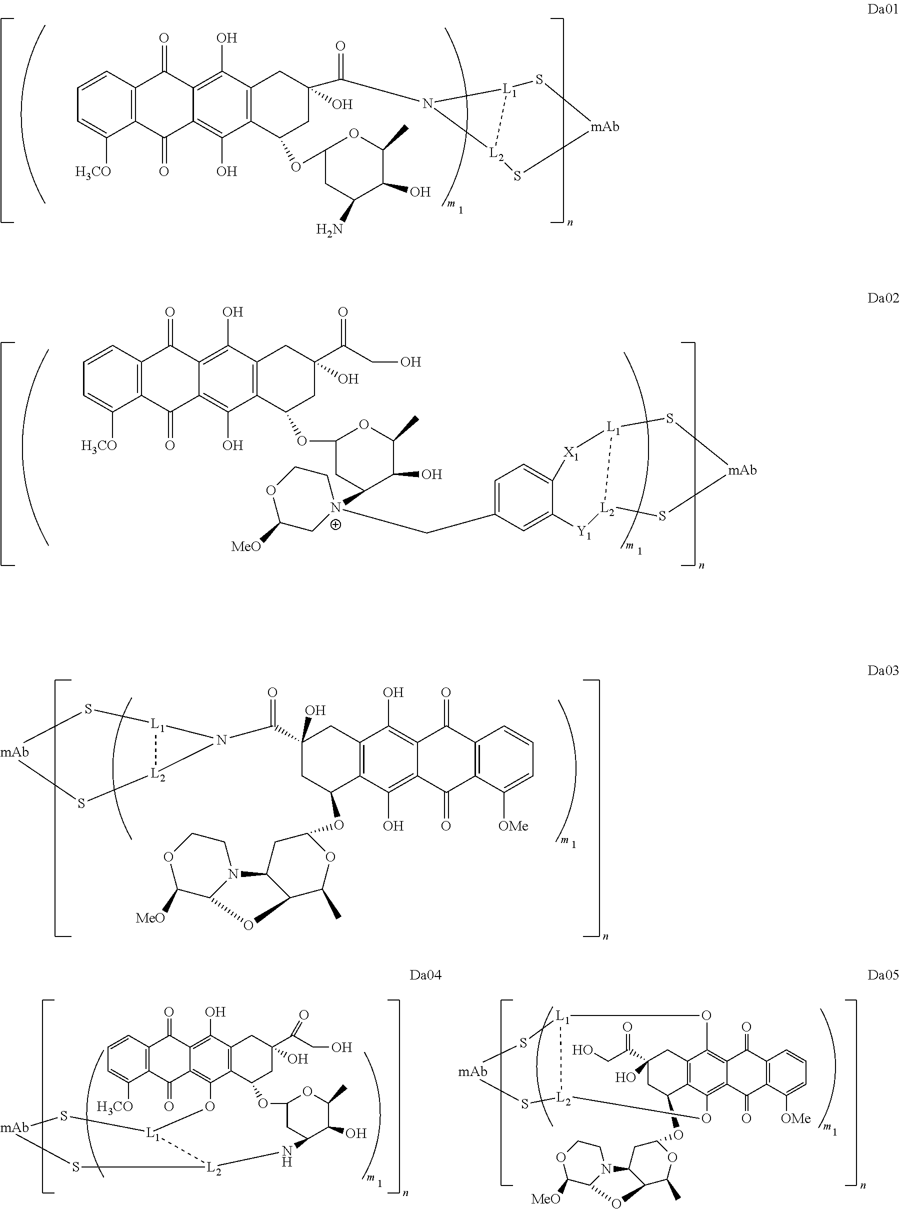

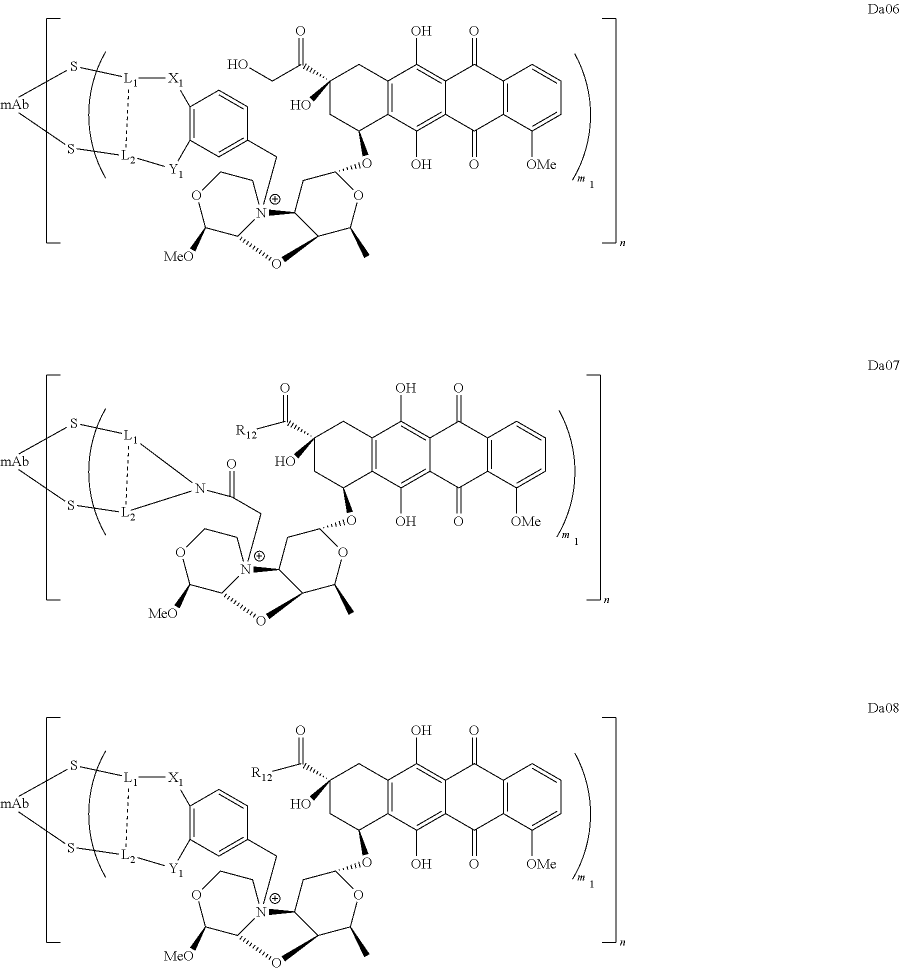

wherein “-----” is optionally either a single bond, or a double bond, or can optionally be absent; X₁, and Y₁ are independently O, NH, NHNH, NR₅, S, C(O)O, C(O)NH, OC(O)NH, OC(O)O, NHC(O)NH, NHC(O)S, OC(O)N(R₁), N(R₁)C(O)N(R₁), CH, C(O)NHNHC(O) and C(O)NR₁; R₁₂ is OH, NH₂, NHR₁, NHNH₂, NHNHCOOH, O—R₁—COOH, NH—R₁—COOH, NH(Aa)ₙCOOH, O(CH₂CH₂O)ₚCH₂CH₂OH, O(CH₂CH₂O)ₚCH₂CH₂NH₂, NH(CH₂CH₂O)ₚCH₂CH₂NH₂, NHOH, NHOR₁, O(CH₂CH₂O)ₚCH₂CH₂COOH, NH(CH₂CH₂O)ₚCH₂CH₂COOH, NH—Ar—COOH, NH—Ar—NH₂, O(CH₂CH₂O)ₚCH₂CH₂NHSO₃H, NH(CH₂CH₂O)ₚCH₂CH₂NH—SO₃H, R₁—NHSO₃H, NH—R₁—NHSO₃H, O(CH₂CH₂O)ₚCH₂CH₂NHPO₃H₂, NH(CH₂CH₂O)ₚCH₂CH₂NHPO₃H₂, OR₁, R₁—NHPO₃H₂, R₁—OPO₃H₂, O(CH₂CH₂O)ₚCH₂CH₂OPO₃H₂, OR₁—NHPO₃H₂, NH—R₁—NHPO₃H₂, NH(CH₂CH₂NH)ₚCH₂CH₂NH₂, NH(CH₂CH₂S)ₚCH₂CH₂NH₂, NH(CH₂CH₂NH)ₚCH₂CH₂OH, NH(CH₂CH₂S)ₚCH₂CH₂OH, NH—R₁—NH₂, or NH(CH₂CH₂O)ₚCH₂CH₂NHPO₃H₂, wherein Aa is 1-8 aminoacids; p is 1-5000; mAb is antibody, preferably monoclonal antibody; n and m₁ are independently 1-20; R₁, L₁, and L₂ are the same defined in Formula (I).

Auristatins and dolastatins are preferred in conjugation containing the bis-linkers of this patent. The auristatins (e. g. auristatin E (AE) auristatin EB (AEB), auristatin EFP (AEFP), monomethyl auristatin E (MMAE), Monomethylauristatin (MMAF), Auristatin F phenylene diamine (AFP) and a phenylalanine variant of MMAE) which are synthetic analogs of dolastatins, are described in Int. J. Oncol. 15: 367-72 (1999); Molecular Cancer Therapeutics, vol. 3, No. 8, pp. 921-32 (2004); U.S. Application Nos. 11134826, 20060074008, 2006022925. U.S. Pat. Nos. 4,414,205, 4,753,894, 4,764,368, 4,816,444, 4,879,278, 4,943,628, 4,978,744, 5,122,368, 5,165,923, 5,169,774, 5,286,637, 5,410,024, 5,521,284, 5,530,097, 5,554,725, 5,585,089, 5,599,902, 5,629,197, 5,635,483, 5,654,399, 5,663,149, 5,665,860, 5,708,146, 5,714,586, 5,741,892, 5,767,236, 5,767,237, 5,780,588, 5,821,337, 5,840,699, 5,965,537, 6,004,934, 6,033,876, 6,034,065, 6,048,720, 6,054,297, 6,054,561, 6,124,431, 6,143,721, 6,162,930, 6,214,345, 6,239,104, 6,323,315, 6,342,219, 6,342,221, 6,407,213, 6,569,834, 6,620,911, 6,639,055, 6,884,869, 6,913,748, 7,090,843, 7,091,186, 7,097,840, 7,098,305, 7,098,308, 7,498,298, 7,375,078, 7,462,352, 7,553,816, 7,659,241, 7,662,387, 7,745,394, 7,754,681, 7,829,531, 7,837,980, 7,837,995, 7,902,338, 7,964,566, 7,964,567, 7,851,437, 7,994,135. Examples of the structures of the conjugate of the antibody-auristatins via the linker of the patent are as the following Au01, Au02, Au03, Au04, Au05, Au06, Au07, Au08, Au09, Au10, Au11, Au12 and Au13

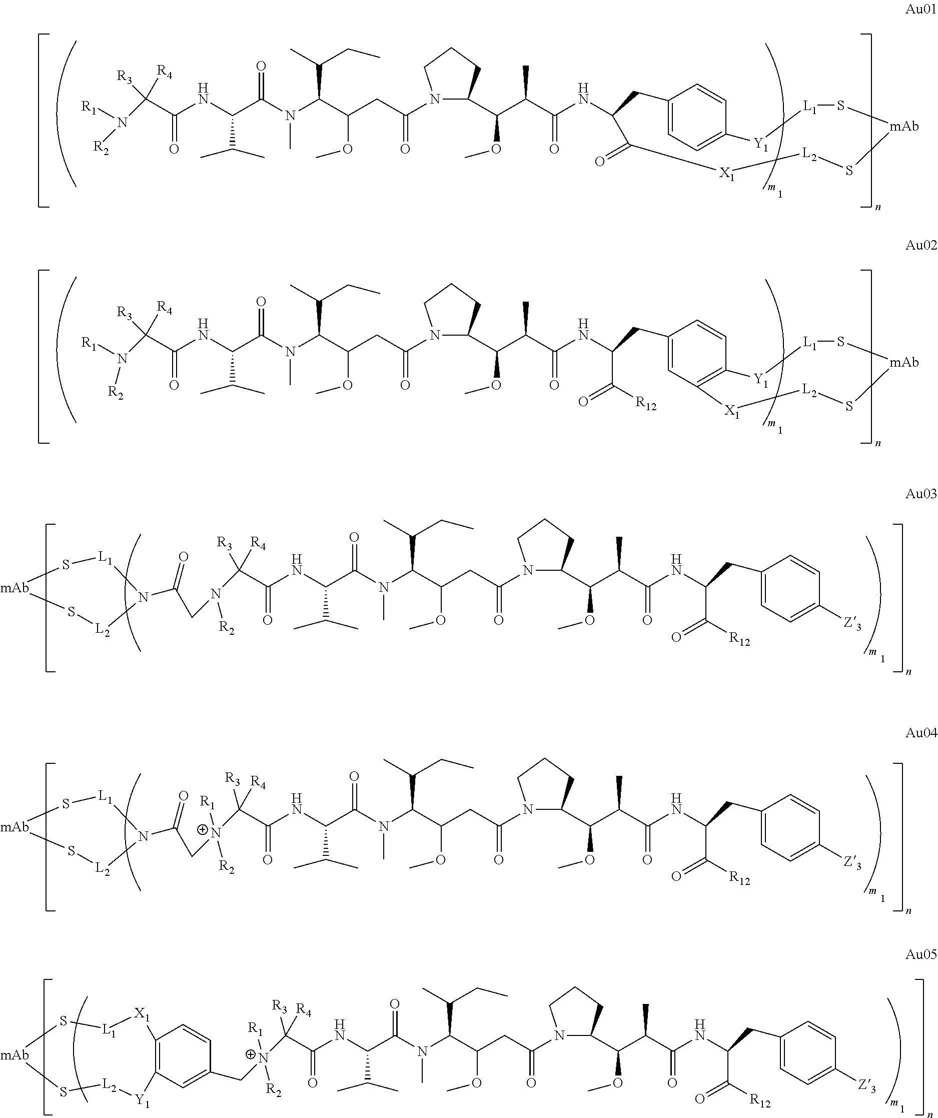

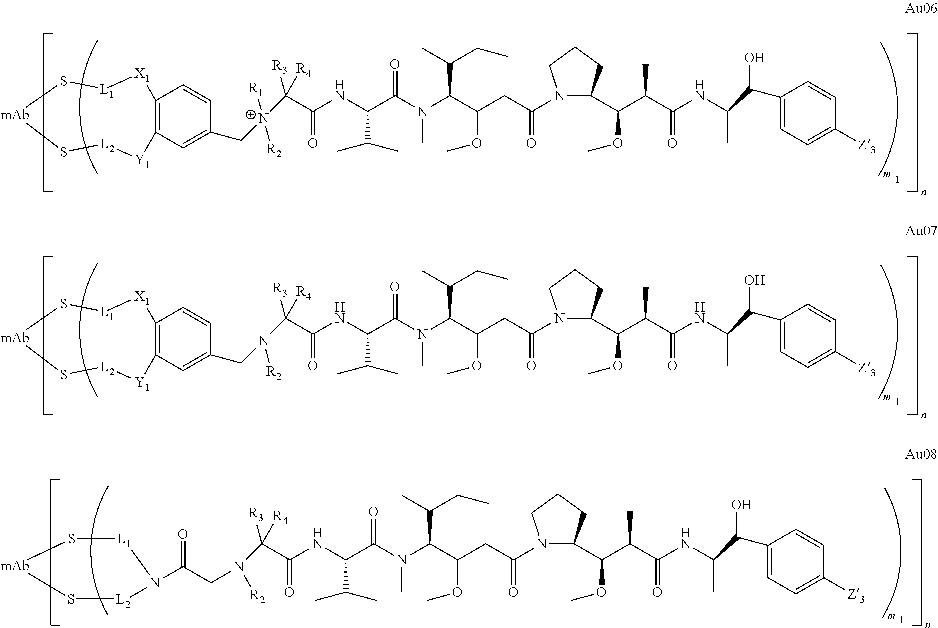

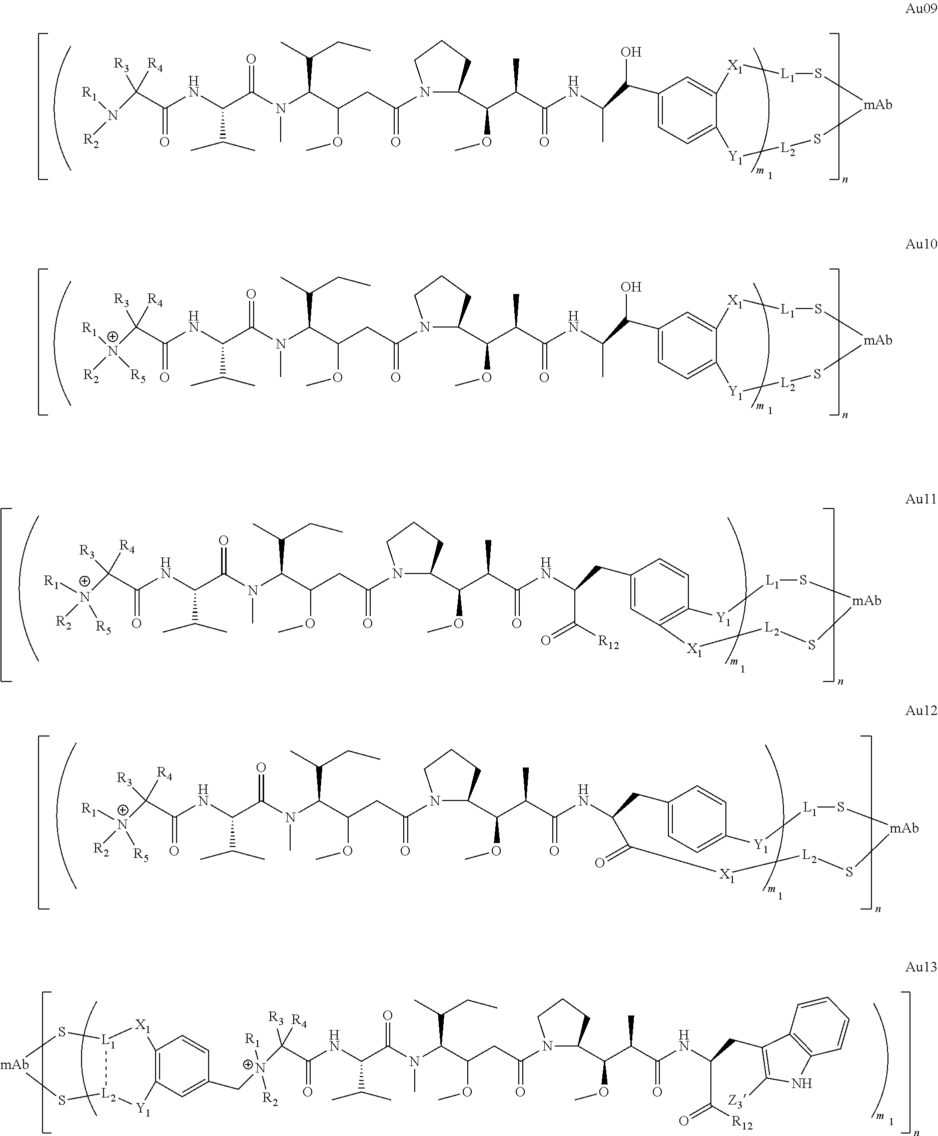

wherein “-----” is optionally either a single bond, or a double bond, or can optionally be absent; X₁ and Y₁ are independently O, NH, NHNH, NR₅, S, C(O)O, C(O)NH, OC(O)NH, OC(O)O, NHC(O)NH, NHC(O)S, OC(O)N(R₁), N(R₁)C(O)N(R₁), CH, C(O)NHNHC(O) and C(O)NR₁; R₁₂ is OH, NH₂, NHR₁, NHNH₂, NHNHCOOH, O—R₁—COOH, NH—R₁—COOH, NH-(Aa)ₙCOOH, O(CH₂CH₂O)ₚCH₂CH₂OH, O(CH₂CH₂O)ₚCH₂CH₂NH₂, NH(CH₂CH₂O)ₚCH₂CH₂NH₂, NR₁R₁′, NHOH, NHOR₁, O(CH₂CH₂O)ₚCH₂CH₂COOH, NH(CH₂CH₂O)ₚCH₂CH₂COOH, NH—Ar—COOH, NH—Ar—NH₂, O(CH₂CH₂O)ₚCH₂CH₂NHSO₃H, NH(CH₂CH₂O)ₚCH₂CH₂NHSO₃H, R₁—NHSO₃H, NH—R₁—NHSO₃H, O(CH₂CH₂O)ₚCH₂CH₂NHPO₃H₂, NH(CH₂CH₂O)ₚCH₂CH₂NHPO₃H₂, OR₁, R₁—NHPO₃H₂, R₁—OPO₃H₂, O(CH₂CH₂O)ₚCH₂CH₂OPO₃H₂, OR₁—NHPO₃H₂, NH—R₁—NHPO₃H₂, NH(CH₂CH₂NH)ₚCH₂CH₂NH₂, NH(CH₂CH₂S)ₚCH₂CH₂NH₂, NH(CH₂CH₂NH)ₚCH₂CH₂OH, NH(CH₂CH₂S)ₚCH₂CH₂OH, NH—R₁—NH₂, or NH(CH₂CH₂O)ₚCH₂CH₂NHPO₃H₂, wherein Aa is 1-8 aminoacids; p is 1-5000; mAb is antibody, preferably monoclonal antibody; n and m₁ are independently 1-20; p is 1-5000; Preferably R₁, R₂, R₃, and R₄ are independently H; C₁-C₈ lineal or branched alkyl, aryl, heteroaryl, heteroalkyl, alkylcycloalkyl, ester, ether, amide, amines, heterocycloalkyl, or acyloxylamines; or peptides containing 1-8 aminoacids, or polyethyleneoxy unit having formula (OCH₂CH₂)ₚ or (OCH₂CH(CH₃))ₚ, wherein p is an integer from 1 to about 5000. The two Rs: R₁R₂, R₂R₃, R₁R₃ or R₃R₄ can form 3˜8 member cyclic ring of alkyl, aryl, heteroaryl, heteroalkyl, or alkylcycloalkyl group; X₃ is H, CH₃ or X₁′R₁′, wherein X₁′ is NH, N(CH₃), NHNH, O, or S, and R₁′ is H or C₁-C₈ lineal or branched alkyl, aryl, heteroaryl, heteroalkyl, alkylcycloalkyl, acyloxylamines; R_{3′} is H or C₁-C₆ lineal or branched alkyl; Z₃′ is H, COOR₁, NH₂, NHR₁, OR₁, CONHR₁, NHCOR₁, OCOR₁, OP(O)(OM₁)(OM₂), OCH₂OP(O)(OM₁)(OM₂), OSO₃M₁, R₁, or O-glycoside (glucoside, galactoside, mannoside, glucuronoside/glucuronide, alloside, fructoside, etc.), NH-glycoside, S-glycoside or CH₂-glycoside; M₁ and M₂ are independently H, Na, K, Ca, Mg, NH₄, NR₁R₂R₃; Z₁, Z₂, L₁, and L₂ are the same defined in Formula (I).

The benzodiazepine dimers (e. g. dimmers of pyrrolobenzodiazepine (PBD) or (tomaymycin), indolinobenzodiazepines, imidazobenzothiadiazepines, or oxazolidinobenzodiazepines) which are preferred cytotoxic agents according to the present invention are exampled in the art: U.S. Pat. Nos. 8,163,736; 8,153,627; 8,034,808; 7,834,005; 7,741,319; 7,704,924; 7,691,848; 7,678,787; 7,612,062; 7,608,615; 7,557,099; 7,528,128; 7,528,126; 7,511,032; 7,429,658; 7,407,951; 7,326,700; 7,312,210; 7,265,105; 7,202,239; 7,189,710; 7,173,026; 7,109,193; 7,067,511; 7,064,120; 7,056,913; 7,049,311; 7,022,699; 7,015,215; 6,979,684; 6,951,853; 6,884,799; 6,800,622; 6,747,144; 6,660,856; 6,608,192; 6,562,806; 6,977,254; 6,951,853; 6,909,006; 6,344,451; 5,880,122; 4,935,362; 4,764,616; 4,761,412; 4,723,007; 4,723,003; 4,683,230; 4,663,453; 4,508,647; 4,464,467; 4,427,587; 4,000,304; US patent appl. 20100203007, 20100316656, 20030195196. Examples of the structures of the conjugate of the antibody-benzodiazepine dimers via the bridge linker are as the following PB01, PB02, PB03, PB04, PB05, PB06, PB07, PB08, PB09, PB10, PB11, PB12, PB13, PB14, PB15 PB16 PB17 PB18. PB19 PB20. PB21 and PB22.

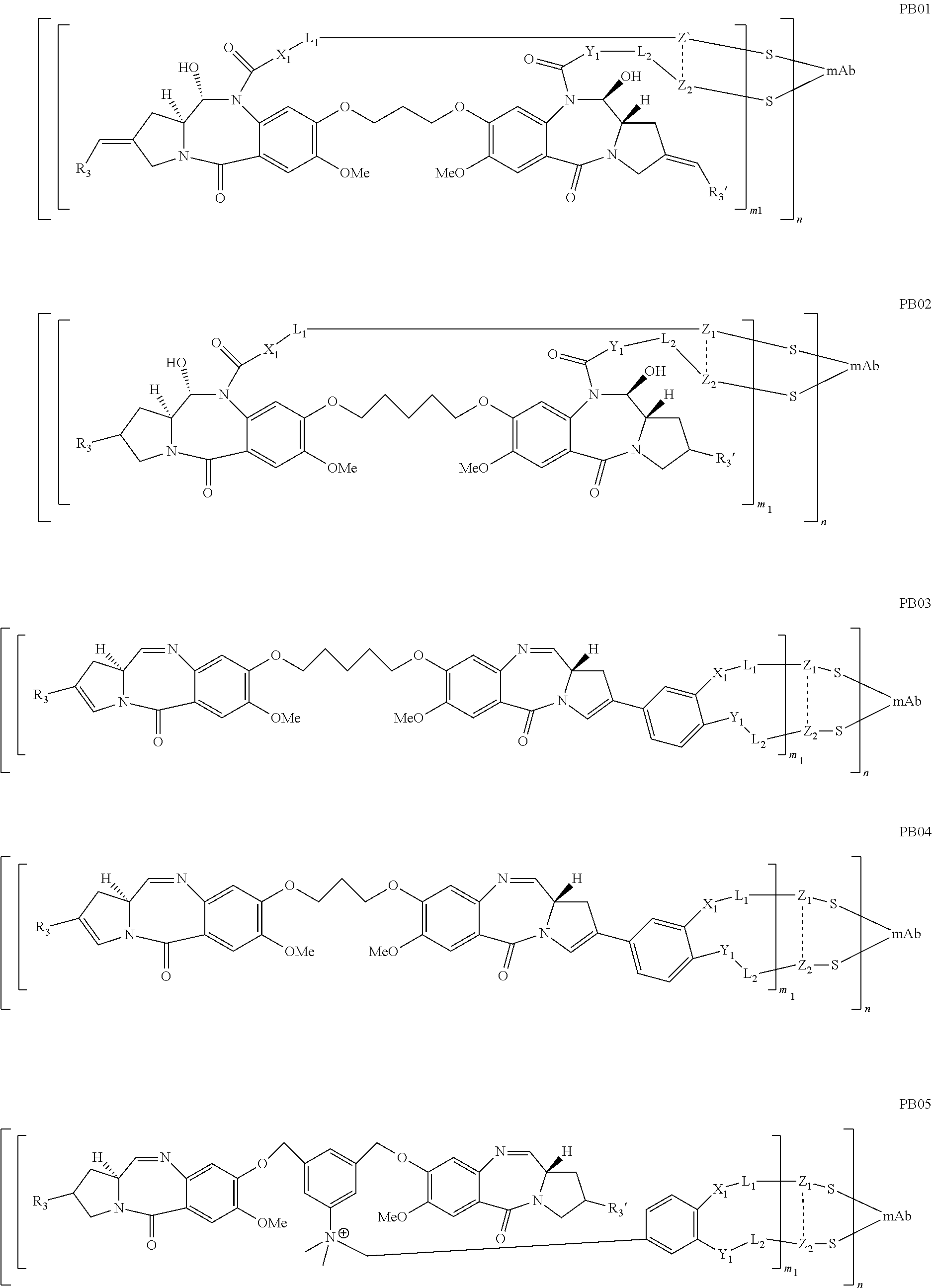

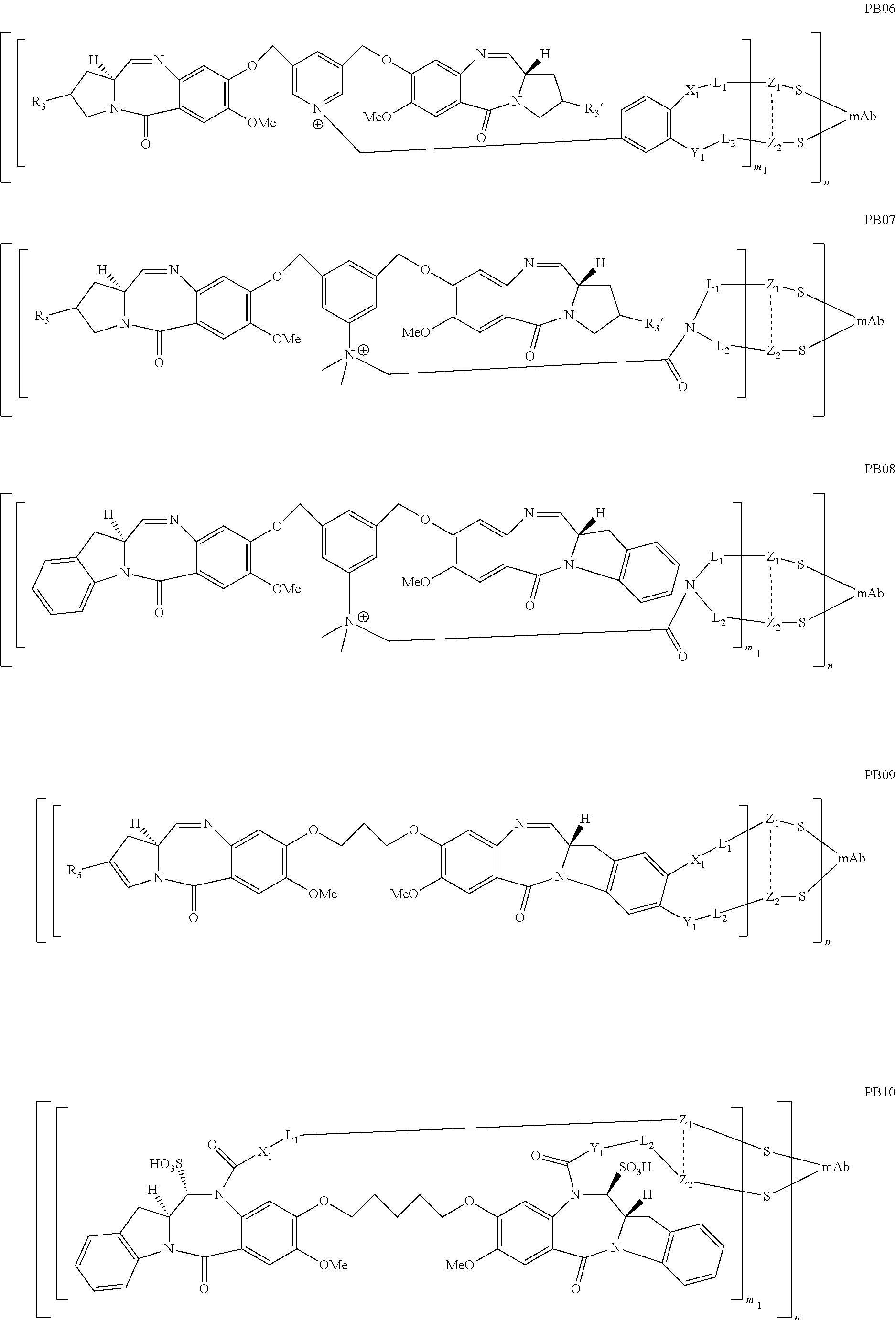

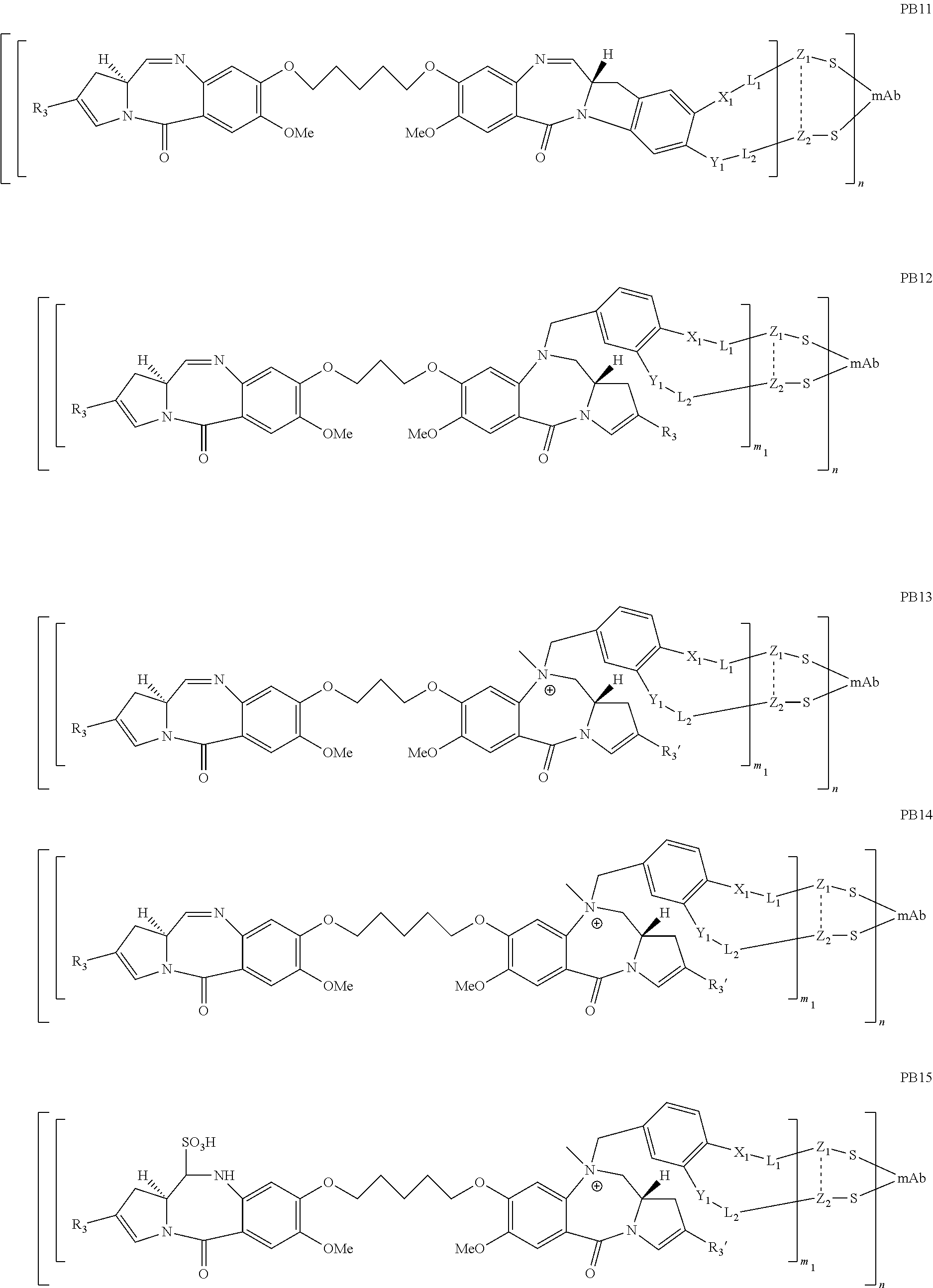

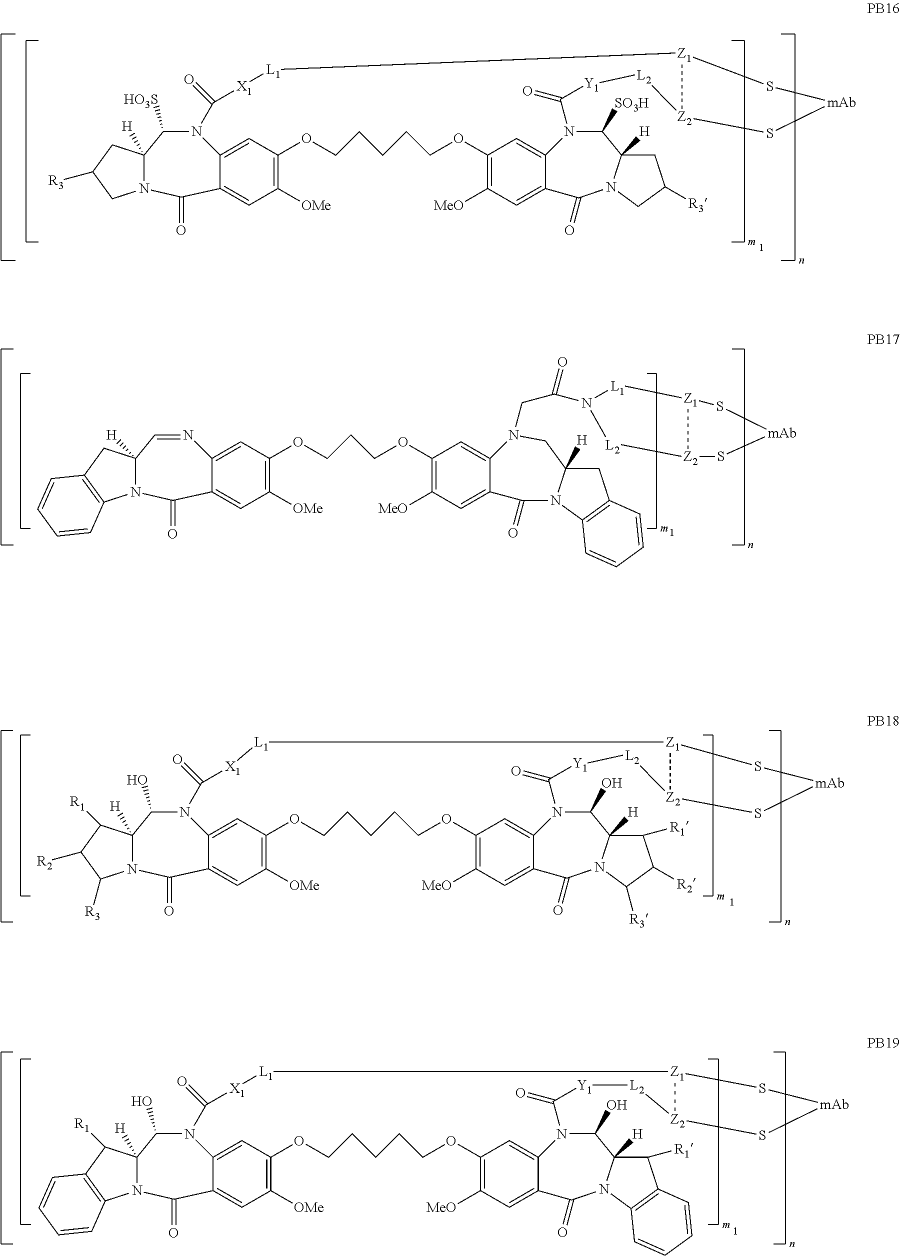

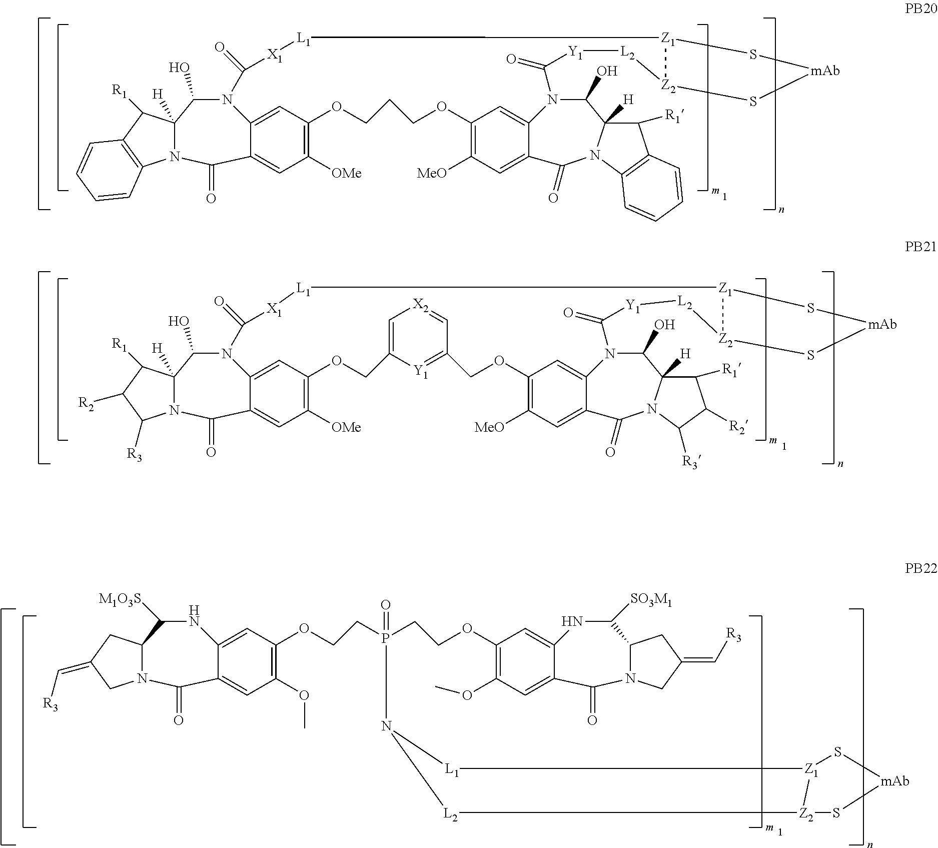

wherein “-----” is optionally either a single bond, or a double bond, or can optionally be absent; X₁, and Y₁ are independently O, NH, NHNH, NR₅, S, C(O)O, C(O)NH, OC(O)NH, OC(O)O, NHC(O)NH, NHC(O)S, OC(O)N(R₁), N(R₁)C(O)N(R₁), CH, C(O)NHNHC(O) and C(O)NR₁; mAb is antibody, preferably monoclonal antibody; n and m₁ are independently 1-20; L₁, L₂, Z₁, and Z₂, are the same defined in Formula (I). R₁, R₂, R₃, R₁′, R₂′, and R₃′ are independently H; F; Cl; ═O; ═S; OH; SH; C₁-C₈ lineal or branched alkyl, aryl, alkenyl, heteroaryl, heteroalkyl, alkylcycloalkyl, ester (COOR₅ or —OC(O)R₅), ether (OR₅), amide (CONR₅), carbamate (OCONR₅), amines (NHR₅, NR₅R₅′), heterocycloalkyl, or acyloxylamines (—C(O)NHOH, —ONHC(O)R₅); or peptides containing 1-8 natural or unnatural aminoacids, or polyethyleneoxy unit of formula (OCH₂CH₂)ₚ or (OCH₂CH(CH₃))ₚ, wherein p is an integer from 1 to about 5000. The two Rs: R₁R₂, R₂R₃, R₁R₃. R₁′R₂′, R₂′R₃′, or R₁′R₃′ can independently form 3˜8 member cyclic ring of alkyl, aryl, heteroaryl, heteroalkyl, or alkylcycloalkyl group; X₂ and Y₂ are independently N, CH₂ or CR₅, wherein R₅ is H, OH, NH₂, NH(CH₃), NHNH₂, COOH, SH, OZ₃, SZ₃, or C₁-C₈ lineal or branched alkyl, aryl, heteroaryl, heteroalkyl, alkylcycloalkyl, acyloxylamines; Z₃ is H, OP(O)(OM₁)(OM₂), OCH₂OP(O)(OM₁)(OM₂), OSO₃M₁, or O-glycoside (glucoside, galactoside, mannoside, glucuronoside/glucuronide, alloside, fructoside, etc.), NH-glycoside, S-glycoside or CH₂-glycoside; M₁ and M₂ are independently H, Na, K, Ca, Mg, NH₄, NR₁R₂R₃.

Amatoxins which are a subgroup of at least ten toxic compounds originally found in several genera of poisonous mushrooms, most notably Amanita phalloides and several other mushroom species, are also preferred for conjugation of the present patent. These ten amatoxins, named α-Amanitin, β-Amanitin, γ-Amanitin, ε-Amanitin, Amanullin, Amanullinic acid, Amaninamide, Amanin, Proamanullin, are rigid bicyclic peptides that are synthesized as 35-amino-acid proproteins, from which the final eight amino acids are cleaved by a prolyl oligopeptidase (Litten, W. 1975 Scientific American 232 (3): 90-101; H. E. Hallen, et al 2007 Proc. Nat. Aca. Sci. USA 104, 19097-101; K. Baumann, et al, 1993 Biochemistry 32 (15): 4043-50; Karlson-Stiber C, Persson H. 2003, Toxicon 42 (4): 339-49; Horgen, P. A. et al. 1978 Arch. Microbio. 118 (3): 317-9). Amatoxins kill cells by inhibiting RNA polymerase II (Pol II), shutting down gene transcription and protein biosynthesis (Brodner, O. G. and Wieland, T. 1976 Biochemistry, 15(16): 3480-4; Fiume, L., Curr Probl Clin Biochem, 1977, 7: 23-8; Karlson-Stiber C, Persson H. 2003, Toxicon 42(4): 339-49; Chafin, D. R., Guo, H. & Price, D. H. 1995 J. Biol. Chem. 270 (32): 19114-19; Wieland (1983) Int. J. Pept. Protein Res. 22(3): 257-76.). Amatoxins can be produced from collected Amanita phalloides mushrooms (Yocum, R. R. 1978 Biochemistry 17(18): 3786-9; Zhang, P. et al, 2005, FEMS Microbiol. Lett. 252(2), 223-8), or from fermentation using a basidiomycete (Muraoka, S. and Shinozawa T., 2000 J. Biosci. Bioeng. 89(1): 73-6) or from fermentation using A. fissa (Guo, X. W., et al, 2006 Wei Sheng Wu Xue Bao 46(3): 373-8), or from culturing Galerina fasciculata or Galerina helvoliceps, a strain belonging to the genus (WO/1990/009799, JP11137291). However the yields from these isolation and fermentation were quite low (less than 5 mg/L culture). Several preparations of amatoxins and their analogs have been reported in the past three decades (W. E. Savige, A. Fontana, Chem. Commun. 1976, 600-1; Zanotti, G., et al, Int J Pept Protein Res, 1981. 18(2): 162-8; Wieland, T., et al, Eur. J. Biochem. 1981, 117, 161-4; P. A. Bartlett, et al, Tetrahedron Lett. 1982, 23, 619-22; Zanotti, G., et al., Biochim Biophys Acta, 1986. 870(3): 454-62; Zanotti, G., et al., Int. J. Peptide Protein Res. 1987, 30, 323-9; Zanotti, G., et al., Int. J. Peptide Protein Res. 1987, 30, 450-9; Zanotti, G., et al., Int J Pept Protein Res, 1988. 32(1): 9-20; G. Zanotti, T. et al, Int. J. Peptide Protein Res. 1989, 34, 222-8; Zanotti, G., et al., Int J Pept Protein Res, 1990. 35(3): 263-70; Mullersman, J. E. and J. F. Preston, 3rd, Int J Pept Protein Res, 1991. 37(6): 544-51; Mullersman, J. E., et al, Int J Pept Protein Res, 1991. 38(5): 409-16; Zanotti, G., et al, Int J Pept Protein Res, 1992. 40(6): 551-8; Schmitt, W. et al, J. Am. Chem. Soc. 1996, 118, 4380-7; Anderson, M. O., et al, J. Org. Chem., 2005, 70(12): 4578-84; J. P. May, et al, J. Org. Chem. 2005, 70, 8424-30; F. Brueckner, P. Cramer, Nat. Struct. Mol. Biol. 2008, 15, 811-8; J. P. May, D. M. Perrin, Chem. Eur. J. 2008, 14, 3404-9; J. P. May, et al, Chem. Eur. J. 2008, 14, 3410-17; Q. Wang, et al, Eur. J. Org. Chem. 2002, 834-9; May, J. P. and D. M. Perrin, Biopolymers, 2007. 88(5): 714-24; May, J. P., et al., Chemistry, 2008. 14(11): 3410-7; S. De Lamo Marin, et al, Eur. J. Org. Chem. 2010, 3985-9; Pousse, G., et al., Org Lett, 2010. 12(16): 3582-5; Luo, H., et al., Chem Biol, 2014. 21(12): 1610-7; Zhao, L., et al., Chembiochem, 2015. 16(10): 1420-5) and most of these preparations were by partial synthesis. Because of their extreme potency and unique mechanism of cytotoxicity, amatoxins have been used as payloads for conjugations (Fiume, L., Lancet, 1969. 2 (7625): 853-4; Barbanti-Brodano, G. and L. Fiume, Nat New Biol, 1973. 243(130): 281-3; Bonetti, E., M. et al, Arch Toxicol, 1976. 35(1): p. 69-73; Davis, M. T., Preston, J. F. Science 1981, 213, 1385-1388; Preston, J. F., et al, Arch Biochem Biophys, 1981. 209(1): 63-71; H. Faulstich, et al, Biochemistry 1981, 20, 6498-504; Barak, L. S., et al., Proc Natl Acad Sci USA, 1981. 78(5): 3034-8; Faulstich, H. and L. Fiume, Methods Enzymol, 1985. 112: 225-37; Zhelev, Z., A. et al, Toxicon, 1987. 25(9): 981-7; Khalacheva, K., et al, Eksp Med Morfol, 1990. 29(3): 26-30; U. Bermbach, H. Faulstich, Biochemistry 1990, 29, 6839-45; Mullersman, J. E. and J. F. Preston, Int. J. Peptide Protein Res. 1991, 37, 544-51; Mullersman, J. E. and J. F. Preston, Biochem Cell Biol, 1991. 69(7): 418-27; J. Anderl, H. Echner, H. Faulstich, Beilstein J. Org. Chem. 2012, 8, 2072-84; Moldenhauer, G., et al, J. Natl. Cancer Inst. 2012, 104, 622-34; A. Moshnikova, et al; Biochemistry 2013, 52, 1171-8; Zhao, L., et al., Chembiochem, 2015. 16(10): 1420-5; Zhou, B., et al., Biosens Bioelectron, 2015. 68: 189-96; WO2014/043403, US20150218220, EP 1661584). We have been working on the conjugation of amatoxins for a while. Examples of the structures of the conjugate of the antibody-amatoxins via the bridge linker are preferred as the following structures of Am01, Am02, Am03, and Am04.

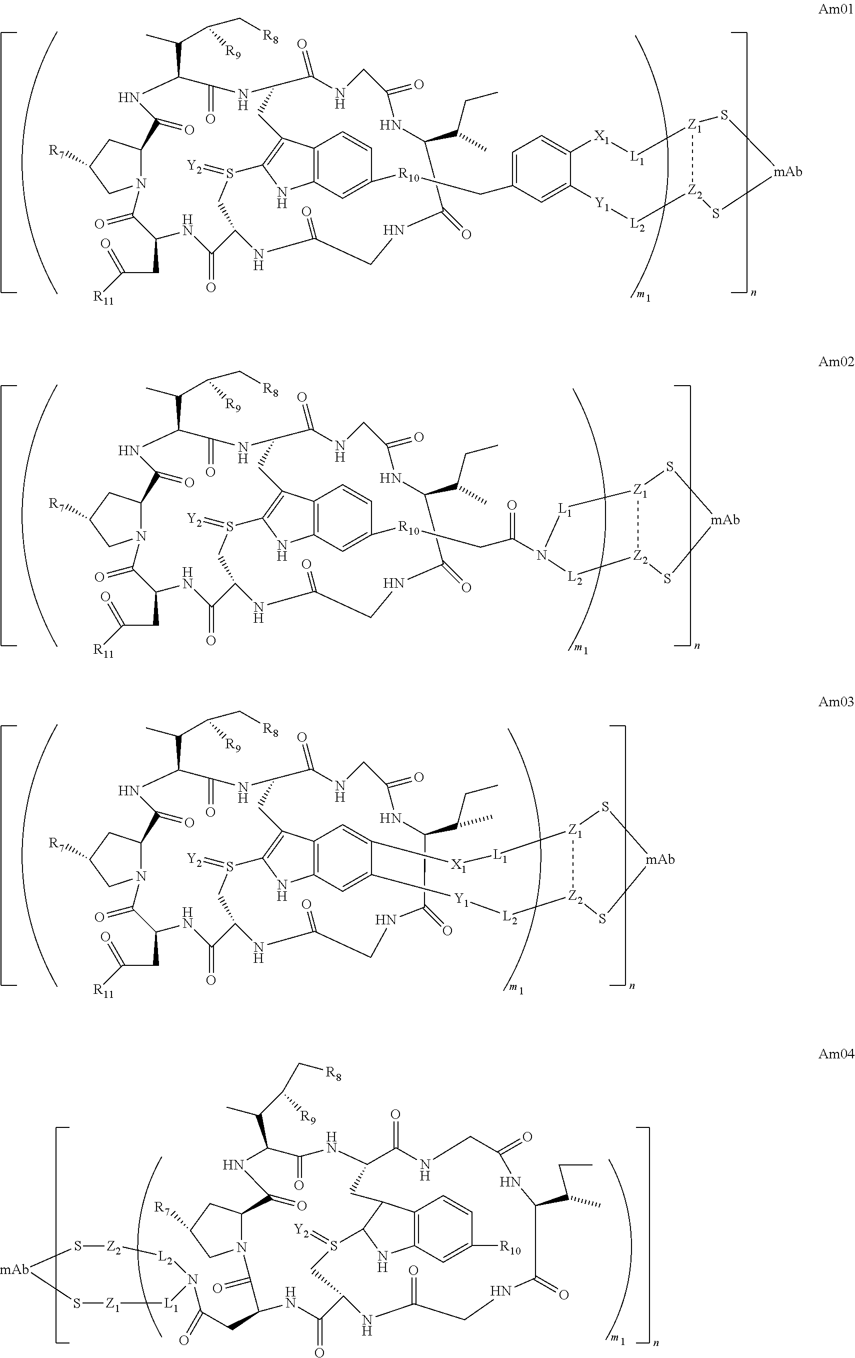

wherein “-----” is optionally either a single bond, or a double bond, or can optionally be absent; X₁, and Y₁ are independently O, NH, NHNH, NR₅, S, C(O)O, C(O)NH, OC(O)NH, OC(O)O, NHC(O)NH, NHC(O)S, OC(O)N(R₁), N(R₁)C(O)N(R₁), CH, C(O)NHNHC(O) and C(O)NR₁; mAb is antibody, preferably monoclonal antibody; n and m₁ are independently 1-20; R₇, R₈, and R₉ are independently H, OH, OR₁, NH₂, NHR₁, C₁-C₆ alkyl, or absent; Y₂ is O, O₂, NR₁, NH, or absent; R₁₀ is CH₂, O, NH, NR₁, NHC(O), NHC(O)NH, NHC(O)O, OC(O)O, C(O), OC(O), OC(O)(NR₁), (NR₁)C(O)(NR₁), C(O)R₁ or absent; R₁₁ is OH, NH₂, NHR₁, NHNH₂, NHNHCOOH, O—R₁—COOH, NH—R₁—COOH, NH-(Aa)ₙCOOH, O(CH₂CH₂O)ₚCH₂CH₂OH, O(CH₂CH₂O)ₚCH₂CH₂NH₂, NH(CH₂CH₂O)ₚCH₂CH₂NH₂, NR₁R₁′, O(CH₂CH₂O)ₚCH₂CH₂COOH, NH(CH₂CH₂O)ₚCH₂CH₂COOH, NH—Ar—COOH, NH—Ar—NH₂, O(CH₂CH₂O)ₚCH₂CH₂NHSO₃H, NH(CH₂CH₂O)ₚCH₂CH₂NHSO₃H, R₁—NHSO₃H, NH—R₁—NHSO₃H, O(CH₂CH₂O)ₚCH₂CH₂NHPO₃H₂, NH(CH₂CH₂O)ₚCH₂CH₂NHPO₃H₂, OR₁, R₁—NHPO₃H₂, R₁—OPO₃H₂, O(CH₂CH₂O)ₚCH₂CH₂OPO₃H₂, OR₁—NHPO₃H₂, NH—R₁—NHPO₃H₂, or NH(CH₂CH₂O)ₚCH₂CH₂NHPO₃H₂, wherein Aa is 1-8 aminoacids; n and m₁ are independently 1-20; p is 1-5000; R₁, L₁, and L₂ are the same defined in Formula (I). L₁, L₂, R₁, Z₁, and Z₂, are the same defined in Formula (I).

In yet another embodiment, an immunotoxin can be conjugated to a cell-binding molecule via a bis-linker of the patent. An immunotoxin herein is a macromolecular drug which is usually a cytotoxic protein derived from a bacterial or plant protein, such as Diphtheria toxin (DT), Cholera toxin (CT), Trichosanthin (TCS), Dianthin, Pseudomonas exotoxin A (ETA′), Erythrogenic toxins, Diphtheria toxin, AB toxins, Type III exotoxins, etc. It also can be a highly toxic bacterial pore-forming protoxin that requires proteolytic processing for activation. An example of this protoxin is proaerolysin and its genetically modified form, topsalysin. Topsalysin is a modified recombinant protein that has been engineered to be selectively activated by an enzyme in the prostate, leading to localized cell death and tissue disruption without damaging neighboring tissue and nerves.

In yet another embodiment, cell-binding ligands or cell receptor agonists can be conjugated to a cell-binding molecule via a bis-linker of this patent. These conjugated cell-binding ligands or cell receptor agonists, in particular, antibody-receptor conjugates, can be not only to work as a targeting conductor/director to deliver the conjugate to malignant cells, but also be used to modulate or co-stimulate a desired immune response or altering signaling pathways.

In the immunotherapy, the cell-binding ligands or receptor agonists are preferred to conjugate to an antibody of TCR (T cell receptors) T cell, or of CARs (chimeric antigen receptors) T cells, or of B cell receptor (BCR), Natural killer (NK) cells, or the cytotoxic cells. Such antibody is preferably anti-CD3, CD4, CD8, CD16 (FcγRIII), CD27, CD40, CD40L, CD45RA, CD45RO, CD56, CD57, CD57^{bright}, TNFβ, Fas ligand, MHC class I molecules (HLA-A, B, C), or NKR-P1. The cell-binding ligands or receptor agonists are selected, but not limited, from: Folate derivatives (binding to the folate receptor, a protein over-expressed in ovarian cancer and in other malignancies) (Low, P. S. et al 2008, Acc. Chem. Res. 41, 120-9); Glutamic acid urea derivatives (binding to the prostate specific membrane antigen, a surface marker of prostate cancer cells) (Hillier, S. M. et al, 2009, Cancer Res. 69, 6932-40); Somatostatin (also known as growth hormone-inhibiting hormone (GHIH) or sonmatotropin release-inhibiting factor (SRIF)) or somatotropin release-inhibiting hormone) and its analogues such as octreotide (Sandostatin) and lanreotide (Somatuline) (particularly for neuroendocrine tumors, GH-producing pituitary adenoma, paraganglioma, nonfunctioning pituitary adenoma, pheochromocytomas) (Ginj, M., et al, 2006, Proc. Natl. Acad. Sci. U.S.A. 103, 16436-41). In general, Somatostatin and its receptor subtypes (sst1, sst2, sst3, sst4, and sst5) have been found in many types of tumors, such as neuroendocrine tumors, in particular in GH-secreting pituitaryadenomas (Reubi J. C., Landolt, A. M. 1984 J. Clin. Endocrinol Metab 59: 1148-51; Reubi J. C., Landolt A. M. 1987 J Clin Endocrinol Metab 65: 65-73; Moyse E, et al, J Clin Endocrinol Metab 61: 98-103) and gastroenteropancreatic tumors (Reubi J. C., et al, 1987 J Clin Endocrinol Metab 65: 1127-34; Reubi, J. C, et al, 1990 Cancer Res 50: 5969-77), pheochromocytomas (Epel-baum J, et al 1995 J Clin Endocrinol Metab 80:1837-44; Reubi J. C., et al, 1992 J Clin Endocrinol Metab 74: 1082-9), neuroblastomas (Prevost G, 1996 Neuroendocrinology 63:188-197; Moertel, C. L, et al 1994 Am J Clin Path 102:752-756), medullary thyroid cancers (Reubi, J. C, et al 1991 Lab Invest 64:567-573) small cell lung cancers (Sagman U, et al, 1990 Cancer 66:2129-2133), nonneuroendocrine tumors including brain tumors such as meningiomas, medulloblastomas, or gliomas (Reubi J. C., et al 1986 J Clin Endocrinol Metab 63: 433-8; Reubi J. C., et al 1987 Cancer Res 47: 5758-64; Fruhwald, M. C, et al 1999 Pediatr Res 45: 697-708), breast carcinomas (Reubi J. C., et al 1990 Int J Cancer 46: 416-20; Srkalovic G, et al 1990 J Clin Endocrinol Metab 70: 661-669), lymphomas (Reubi J. C., et al 1992, Int J Cancer 50: 895-900), renal cell cancers (Reubi J. C., et al 1992, Cancer Res 52: 6074-6078), mesenchymal tumors (Reubi J. C., et al 1996 Cancer Res 56: 1922-31), prostatic (Reubi J. C., et al 1995, J. Clin. Endocrinol Metab 80: 2806-14; et al 1989, Prostate 14:191-208; Halmos G, et al J. Clin. Endo-crinol Metab 85: 2564-71), ovarian (Halmos, G, et al, 2000 J Clin Endocrinol Metab 85: 3509-12; Reubi J. C., et al 1991 Am J Pathol 138:1267-72), gastric (Reubi J. C., et al 1999, Int J Cancer 81: 376-86; Miller, G. V, 1992 Br J Cancer 66: 391-95), hepatocellular (Kouroumalis E, et al 1998 Gut 42: 442-7; Reubi J. C., et al 1999 Gut 45: 66-774) and nasopharyngeal carcinomas (Loh K. S, et al, 2002 Virchows Arch 441: 444-8); certain Aromatic sulfonamides, specific to carbonic anhydrase IX (a marker of hypoxia and of renal cell carcinoma) (Neri, D., et al, Nat. Rev. Drug Discov. 2011, 10, 767-7); Pituitary adenylate cyclase activating peptides (PACAP) (PAC1) for pheochromocytomas and paragangliomas; Vasoactive intestinal peptides (VIP) and their receptor subtypes (VPAC1, VPAC2) for cancers of lung, stomach, colon, rectum, breast, prostate, pancreatic ducts, liver, urinary bladder and epithelial tumors; α-Melanocyte-stimulating hormone (α-MSH) receptors for various tumors; Cholecystokinin (CCK)/gastrin receptors and their receptor subtypes (CCK1 (formerly CCK-A) and CCK2 for small cell lung cancers, medullary thyroid carcinomas, astrocytomas, insulinomas and ovarian cancers; Bombesin(Pyr-Gln-Arg-Leu-Gly-Asn-Gln-Trp-Ala-Val-Gly-His-Leu-Met-NH₂)/gastrin-releasing peptide (GRP) and their receptor subtypes (BB1, GRP receptor subtype (BB2), the BB3 and BB4) for renal cell, breast, lung, gastric and prostate carcinomas, and neuroblastoma (and neuroblastoma (Ohlsson, B., et al, 1999, Scand. J. Gastroenterology 34 (12): 1224-9; Weber, H. C., 2009, Cur. Opin. Endocri. Diab. Obesity 16(1): 66-71, Gonzalez N, et al, 2008, Cur. Opin. Endocri. Diab. Obesity 15(1), 58-64); Neurotensin receptors and its receptor subtypes (NTR1, NTR2, NTR3) for small cell lung cancer, neuroblastoma, pancreatic, colonic cancer and Ewing sarcoma; Substance P receptors and their receptor subtypes (such as NK1 receptor for Glial tumors, Hennig I. M., et al 1995 Int. J. Cancer 61, 786-792); Neuropeptide Y (NPY) receptors and its receptor subtypes (Y1-Y6) for breast carcinomas; Homing Peptides include RGD (Arg-Gly-Asp), NGR (Asn-Gly-Arg), the dimeric and multimeric cyclic RGD peptides (e.g. cRGDfV) that recognize receptors (integrins) on tumor surfaces (Laakkonen P, Vuorinen K. 2010, Integr Biol (Camb). 2(7-8): 326-337; Chen K, Chen X. 2011, Theranostics. 1:189-200; Garanger E, et al, Anti-Cancer Agents Med Chem. 7 (5): 552-558; Kerr, J. S. et al, Anticancer Research, 19(2A), 959-968; Thumshirn, G, et al, 2003 Chem. Eur. J. 9, 2717-2725), and TAASGVRSMH or LTLRWVGLMS (chondroitin sulfate proteoglycan NG2 receptor) and F3 peptides (31 amino acid peptide that binds to cell surface-expressed nucleolin receptor) (Zitzmann, S., 2002 Cancer Res., 62, 18, pp. 5139-5143, Temminga, K., 2005, Drug Resistance Updates, 8, 381-402; P. Laakkonen and K. Vuorinen, 2010 Integrative Biol, 2(7-8), 326-337; M. A. Burg, 1999 Cancer Res., 59(12), 2869-2874; K. Porkka, et al 2002, Proc. Nat. Acad. Sci. USA 99(11), 7444-9); Cell Penetrating Peptides (CPPs) (Nakase I, et al, 2012, J. Control Release. 159(2), 181-188); Peptide Hormones, such as luteinizing hormone-releasing hormone (LHRH) agonists and antagonists, and gonadotropin-releasing hormone (GnRI) agonist, acts by targeting follicle stimulating hormone (FSH) and luteinising hormone (LH), as well as testosterone production, e.g. buserelin (Pyr-His-Trp-Ser-Tyr-D-Ser(OtBu)-Leu-Arg-Pro-NHEt), Gonadorelin (Pyr-His-Trp-Ser-Tyr-Gly-Leu-Arg-Pro-Gly-NH₂), Goserelin (Pyr-His-Trp-Ser-Tyr-D-Ser(OtBu)-Leu-Arg-Pro-AzGly-NH₂), Histrelin (Pyr-His-Trp-Ser-Tyr-D-His(N-benzyl)-Leu-Arg-Pro-NHEt), leuprolide (Pyr-His-Trp-Ser-Tyr-D-Leu-Leu-Arg-Pro-NHEt), Nafarelin (Pyr-His-Trp-Ser-Tyr-2Nal-Leu-Arg-Pro-Gly-NH₂), Triptorelin (Pyr-His-Trp-Ser-Tyr-D-Trp-Leu-Arg-Pro-Gly-NH₂), Nafarelin, Deslorelin, Abarelix (Ac-D-2Nal-D-4-chloroPhe-D-3-(3-pyridyl)Ala-Ser-(N-Me)Tyr-D-Asn-Leu-isopropylLys-Pro-DAla-NH₂), Cetrorelix (Ac-D-2Nal-D-4-chloro-Phe-D-3-(3-pyridyl)Ala-Ser-Tyr-D-Cit-Leu-Arg-Pro-D-Ala-NH₂), Degarelix (Ac-D-2Nal-D-4-chloroPhe-D-3-(3-pyridyl)Ala-Ser-4-aminoPhe(L-hydroorotyl)-D-4-aminoPhe(carba-moyl)-Leu-isopropylLys-Pro-D-Ala-NH₂), and Ganirelix (Ac-D-2Nal-D-4-chloroPhe-D-3-(3-pyridyl)Ala-Ser-Tyr-D-(N9, N10-diethyl)-homoArg-Leu-(N9, N10-diethyl)-homoArg-Pro-D-Ala-NH₂) (Thundimadathil, J., J. Amino Acids, 2012, 967347, doi:10.1155/2012/967347; Boccon-Gibod, L.; et al, 2011, Therapeutic Advances in Urology 3(3): 127-140; Debruyne, F., 2006, Future Oncology, 2(6), 677-696; Schally A. V; Nagy, A. 1999 Eur J Endocrinol 141:1-14; Koppan M, et al 1999 Prostate 38:151-158); and Pattern Recognition Receptors (PRRs), such as Toll-like receptors (TLRs), C-type lectins and Nodlike Receptors (NLRs) (Fukata, M., et al, 2009, Semin. Immunol. 21, 242-253; Maisonneuve, C., et al, 2014, Proc. Natl. Acad. Sci. U.S.A 111, 1-6; Botos, I., et al, 2011, Structure 19, 447-459; Means, T. K., et al, 2000, Life Sci. 68, 241-258) that range in size from small molecules (imiquimod, guanisine and adenosine analogs) to large and complex biomacromolecules such as lipopolysaccharide (LPS), nucleic acids (CpG DNA, polyI:C) and lipopeptides (Pam3CSK4) (Kasturi, S. P., et al, 2011, Nature 470, 543-547; Lane, T., 2001, J. R. Soc. Med. 94, 316; Hotz, C., and Bourquin, C., 2012, Oncoimmunology 1, 227-228; Dudek, A. Z., et al, 2007, Clin. Cancer Res. 13, 7119-25); Calcitonin receptors which is a 32-amino-acid neuropeptide involved in the regulation of calcium levels largely through its effects on osteoclasts and on the kidney (Zaidi M, et al, 1990 Crit Rev Clin Lab Sci 28, 109-174; Gorn, A. H., et al 1995 J Clin Invest 95:2680-91); And integrin receptors and their receptor subtypes (such as α_{V}β₁, α_{V}β₃, α_{V}β₅, α_{V}β₆, α₆β₄, α₇β₁, α_{L}β₂, α_{IIb}β₃, etc.) which generally play important roles in angiogenesis are expressed on the surfaces of a variety of cells, in particular, of osteoclasts, endothelial cells and tumor cells (Ruoslahti, E. et al, 1994 Cell 77, 477-8; Albelda, S. M. et al, 1990 Cancer Res., 50, 6757-64). Short peptides, GRGDSPK and Cyclic RGD pentapeptides, such as cyclo(RGDfV) (L1) and its derives [cyclo(-N(Me)R-GDfV), cyclo(R-Sar-DfV), cyclo-(RG-N(Me)D-fV), cyclo(RGD-N(Me)f-V), cyclo(RGDf-N(Me)V-)(Cilengitide)] have shown high binding affinities of the intergrin receptors (Dechantsreiter, M. A. et al, 1999 J. Med. Chem. 42, 3033-40, Goodman, S. L., et al, 2002 J. Med. Chem. 45, 1045-51).

The cell-binding ligands or cell receptor agonists can be Ig-based and non-Ig-based protein scaffold molecules. The Ig-Based scaffolds can be selected, but not limited, from Nanobody (a derivative of VHH (camelid Ig)) (Muyldermans S., 2013 Annu Rev Biochem. 82, 775-97); Domain antibodies (dAb, a derivative of VH or VL domain) (Holt, L. J, et al, 2003, Trends Biotechnol. 21, 484-90); Bispecific T cell Engager (BiTE, a bispecific diabody) (Baeuerle, P. A, et al, 2009, Curr. Opin. Mol. Ther. 11, 22-30); Dual Affinity ReTargeting (DART, a bispecific diabody) (Moore P. A. P, et al. 2011, Blood 117(17), 4542-51); Tetravalent tandem antibodies (TandAb, a dimerized bispecific diabody) (Cochlovius, B, et al. 2000, Cancer Res. 60(16):4336-4341). The Non-Ig scaffolds can be selected, but not limited, from Anticalin (a derivative of Lipocalins) (Skerra A. 2008, FEBS J., 275(11): 2677-83; Beste G, et al, 1999 Proc. Nat. Acad. USA. 96(5):1898-903; Skerra, A. 2000 Biochim Biophys Acta, 1482(1-2): 337-50; Skerra, A. 2007, Curr Opin Biotechnol. 18(4): 295-304; Skerra, A. 2008, FEBS J. 275(11):2677-83); Adnectins (10th FN3 (Fibronectin)) (Koide, A, et al, 1998 J. Mol. Biol., 284(4):1141-51; Batori V, 2002, Protein Eng. 15(12): 1015-20; Tolcher, A. W, 2011, Clin. Cancer Res. 17(2): 363-71; Hackel, B. J, 2010, Protein Eng. Des. Sel. 23(4): 211-19); Designed Ankyrin Repeat Proteins (DARPins) (a derivative of ankrin repeat (AR) proteins) (Boersma, Y. L, et al, 2011 Curr Opin Biotechnol. 22(6): 849-57), e.g. DARPin C9, DARPin Ec4 and DARPin E69_LZ3_E01 (Winkler J, et al, 2009 Mol Cancer Ther. 8(9), 2674-83; Patricia M-K. M., et al, Clin Cancer Res. 2011; 17(1):100-10; Boersma Y. L, et al, 2011 J. Biol. Chem. 286(48), 41273-85); Avimers (a domain A/low-density lipoprotein (LDL) receptor) (Boersma Y. L, 2011 J. Biol. Chem. 286(48): 41273-41285; Silverman J, et al, 2005 Nat. Biotechnol., 23(12):1556-61).

Examples of the structures of the conjugate of the antibody-cell-binding ligands or cell receptor agonists or drugs via the bis-linker of the patent application are listed as the following: LB01 (Folate conjugate), LB02 (PMSA ligand conjugate), LB03 (PMSA ligand conjugate), LB04 (PMSA ligand conjugate), LB05 (Somatostatin conjugate), LB06 (Somatostatin conjugate), LB07 (Octreotide, a Somatostatin analog conjugate), LB08 (Lanreotide, a Somatostatin analog conjugate), LB09 (Vapreotide (Sanvar), a Somatostatin analog conjugate), LB10 (CAIX ligand conjugate), LB11 (CAIX ligand conjugate), LB12 (Gastrin releasing peptide receptor (GRPr), MBA conjugate), LB13 (luteinizing hormone-releasing hormone (LH-RH) ligand and GnRH conjugate), LB14 (luteinizing hormone-releasing hormone (LH-RH) and GnRH ligand conjugate), LB15 (GnRH antagonist, Abarelix conjugate), LB16 (cobalamin, vitamin B12 analog conjugate), LB17 (cobalamin, vitamin B12 analog conjugate), LB18 (for αᵥβ₃ integrin receptor, cyclic RGD pentapeptide conjugate), LB19 (hetero-bivalent peptide ligand conjugate for VEGF receptor), LB20 (Neuromedin B conjugate), LB21 (bombesin conjugate for a G-protein coupled receptor), LB22 (TLR₂ conjugate for a Toll-like receptor,), LB23 (for an androgen receptor), LB24 (Cilengitide/cyclo(-RGDfV-) conjugate for an αᵥ intergrin receptor, LB23 (Fludrocortisone conjugate), LB25 (Rifabutin analog conjugate), LB26 (Rifabutin analog conjugate), LB27 (Rifabutin analog conjugate), LB28 (Fludrocortisone conjugate), LB29 (Dexamethasone conjugate), LB30 (fluticasone propionate conjugate), LB31 (Beclometasone dipropionate conjugate), LB32 (Triamcinolone acetonide conjugate), LB33 (Prednisone conjugate), LB34 (Prednisolone conjugate), LB35 (Methylprednisolone conjugate), LB36 (Betamethasone conjugate), LB37 (Irinotecan analog conjugate), LB38 (Crizotinib analog conjugate), LB39 (Bortezomib analog conjugate), LB40 (Carfilzomib analog conjugate), LB41 (Carfilzomib analog conjugate), LB42 (Leuprolide analog conjugate), LB43 (Triptorelin analog conjugate), LB44 (Clindamycin conjugate), LB45 (Liraglutide analog conjugate), LB46 (Semaglutide analog conjugate), LB47 (Retapamulin analog conjugate), LB48 (Indibulin analog conjugate), LB49 (Vinblastine analog conjugate), LB50 (Lixisenatide analog conjugate), LB51 (Osimertinib analog conjugate), LB52 (a neucleoside analog conjugate), LB53 (Erlotinib analog conjugate) and LB54 (Lapatinib analog conjugate) which are shown in the following structures:

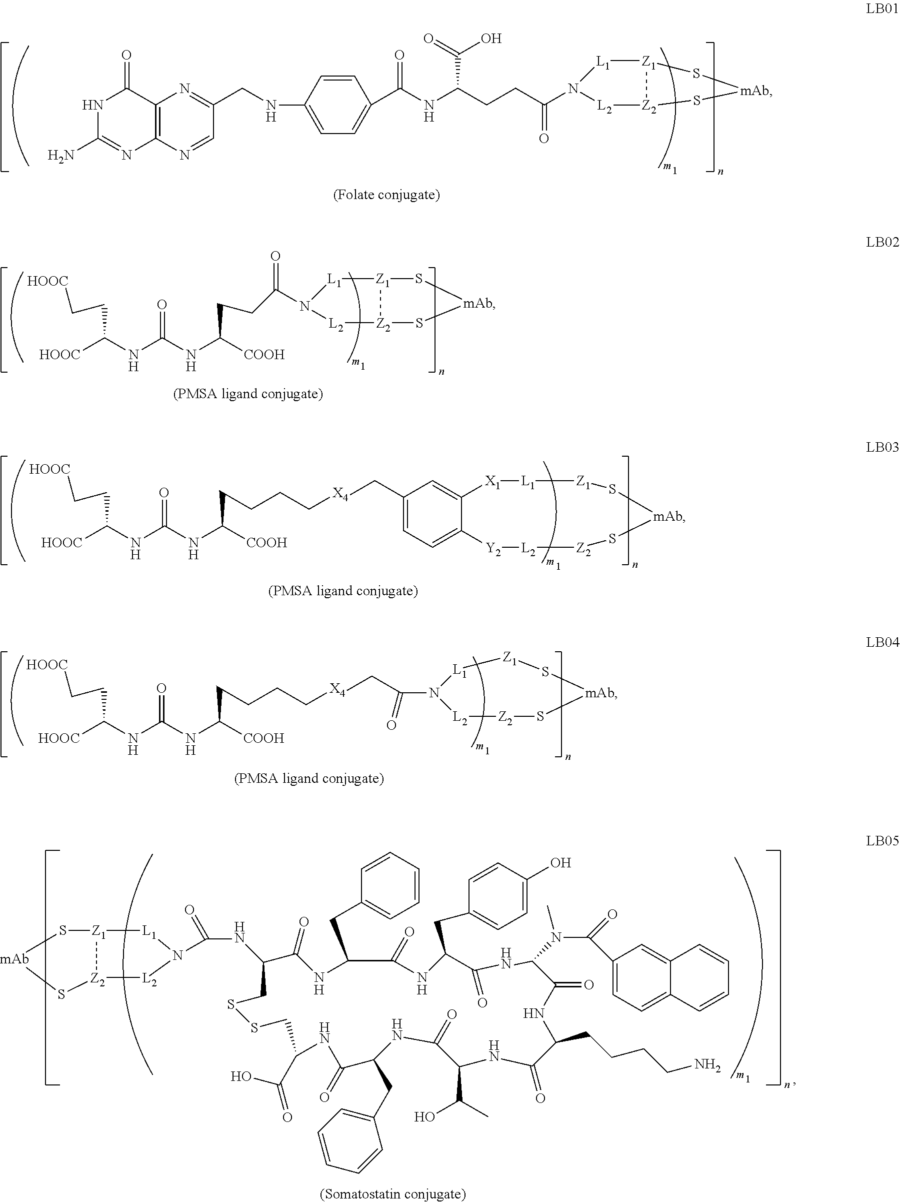

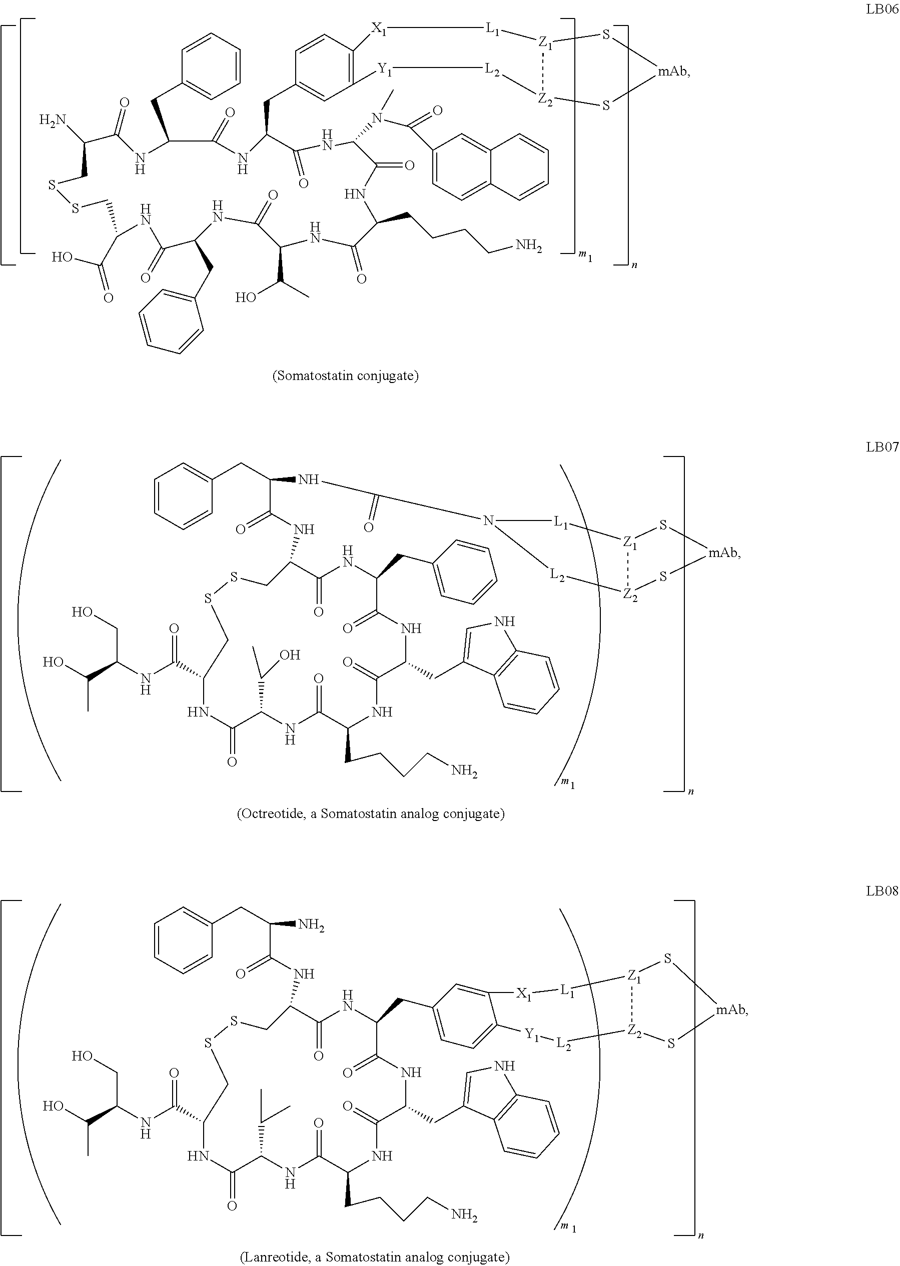

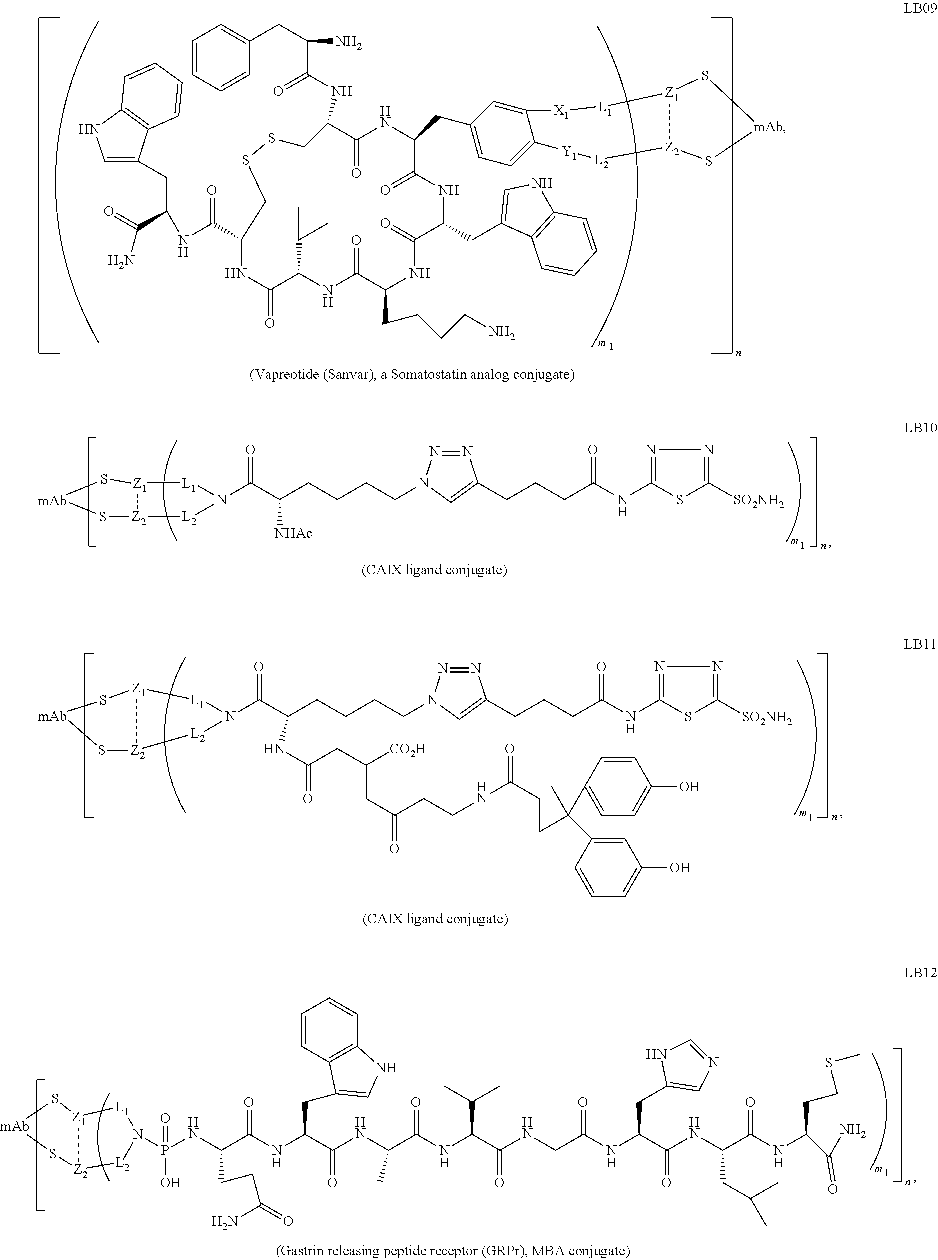

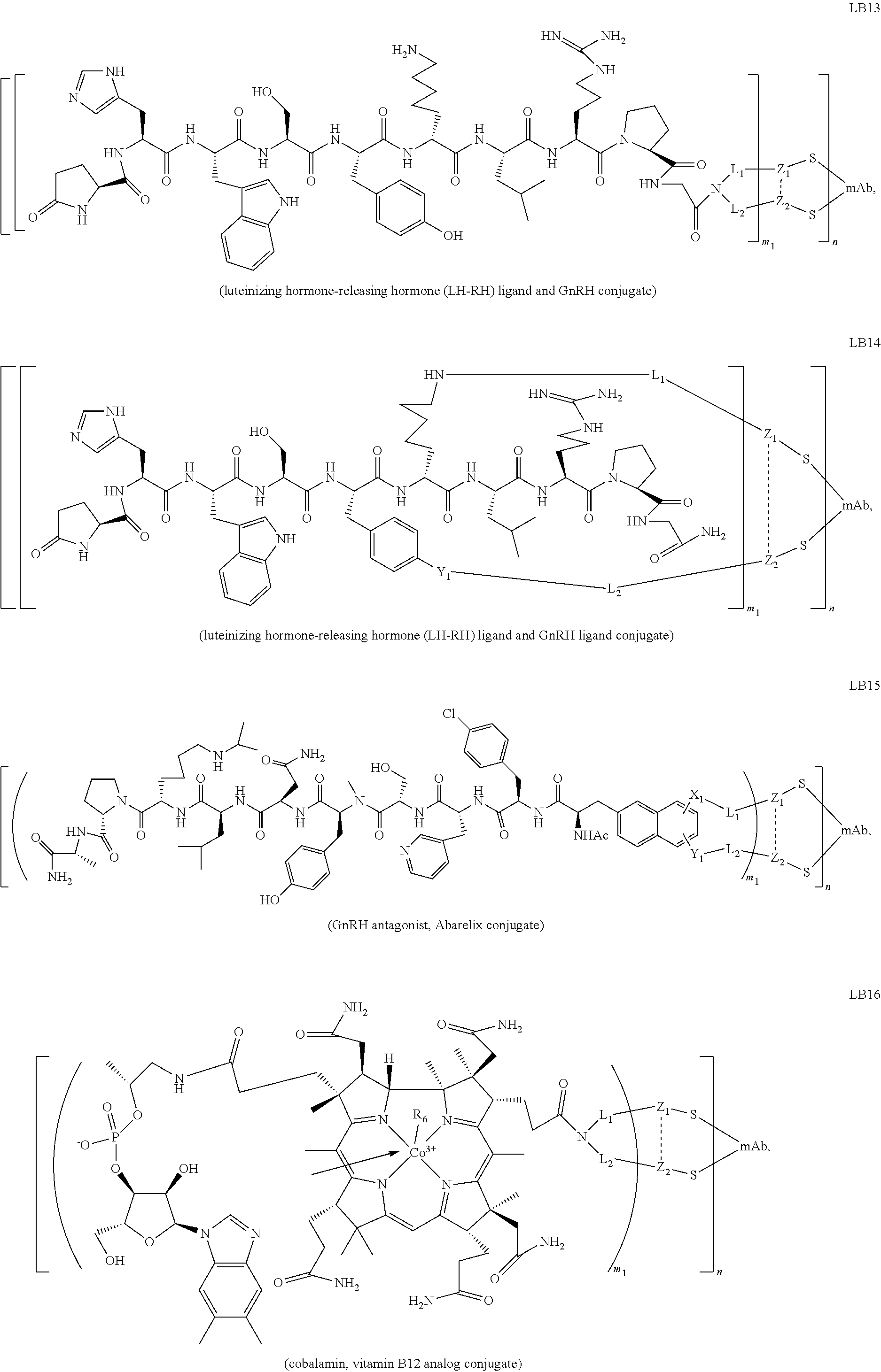

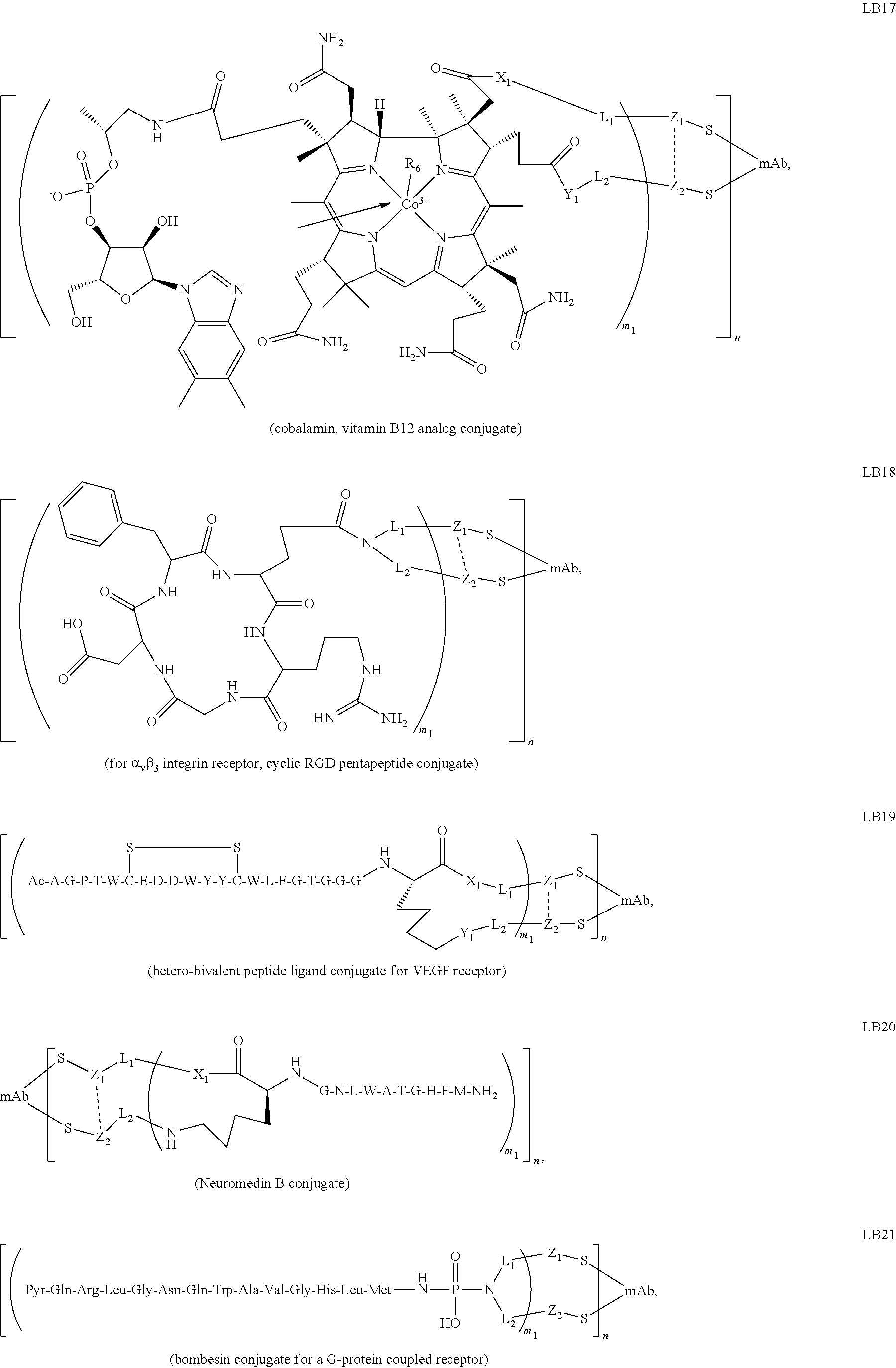

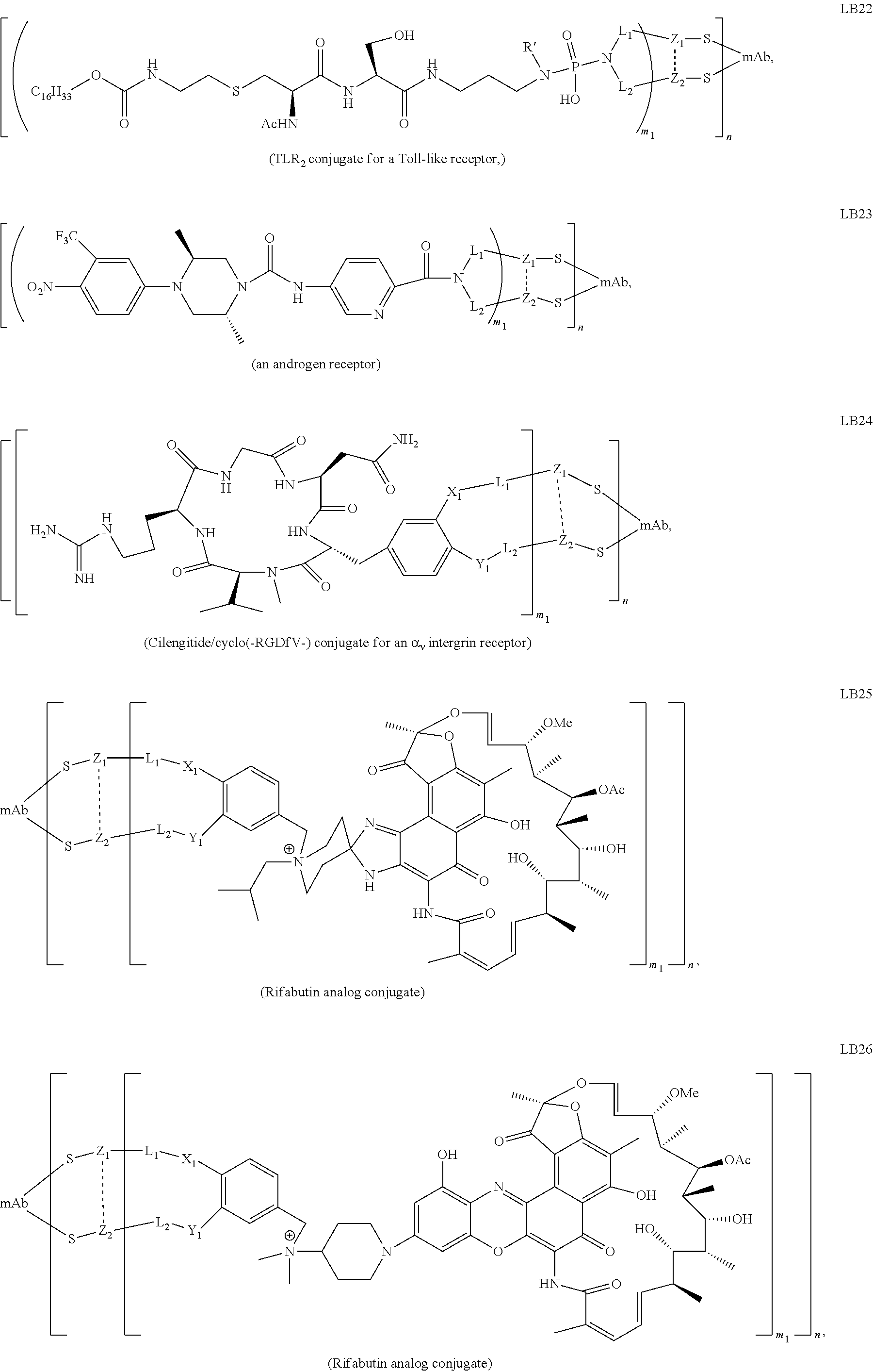

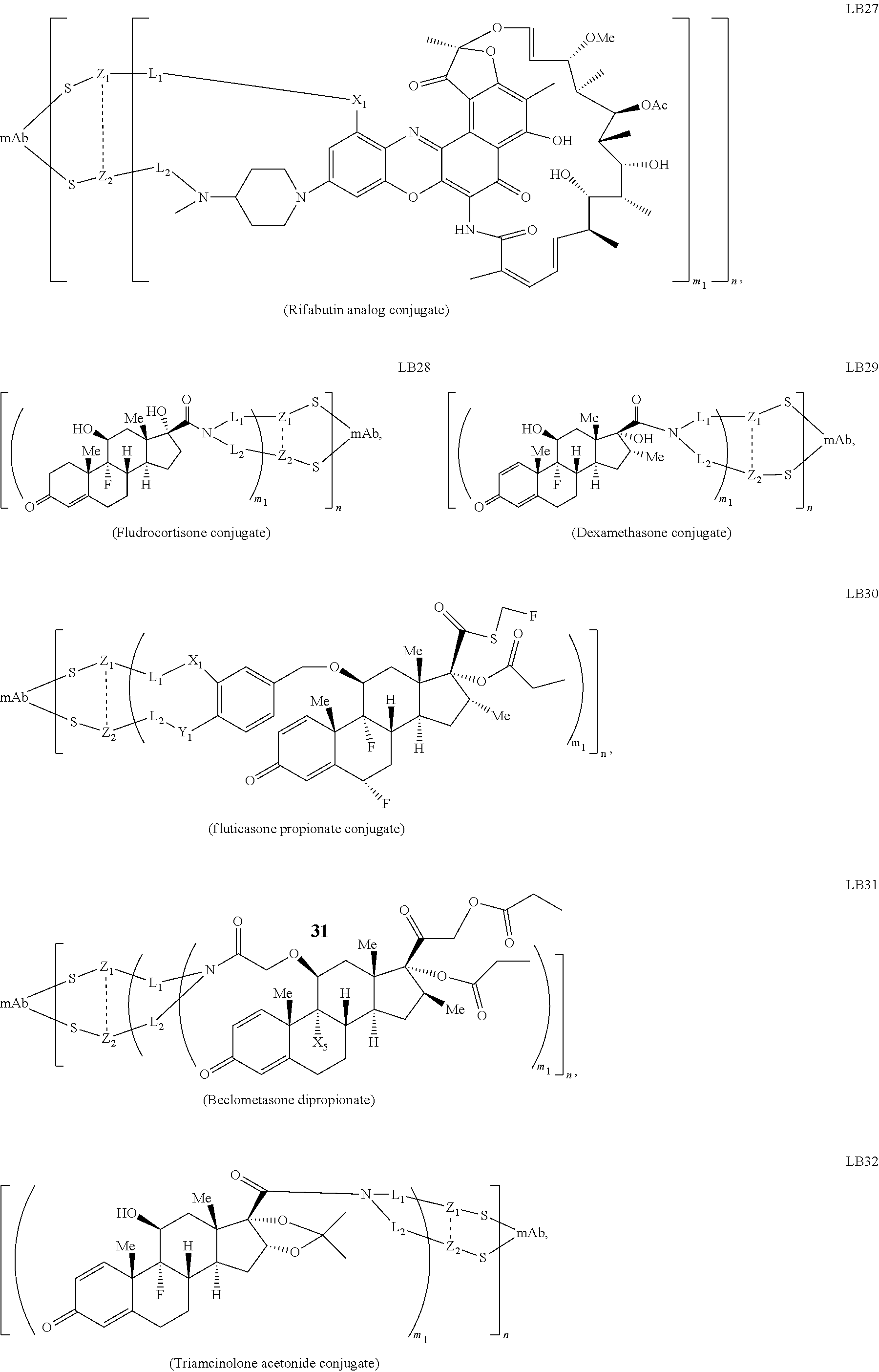

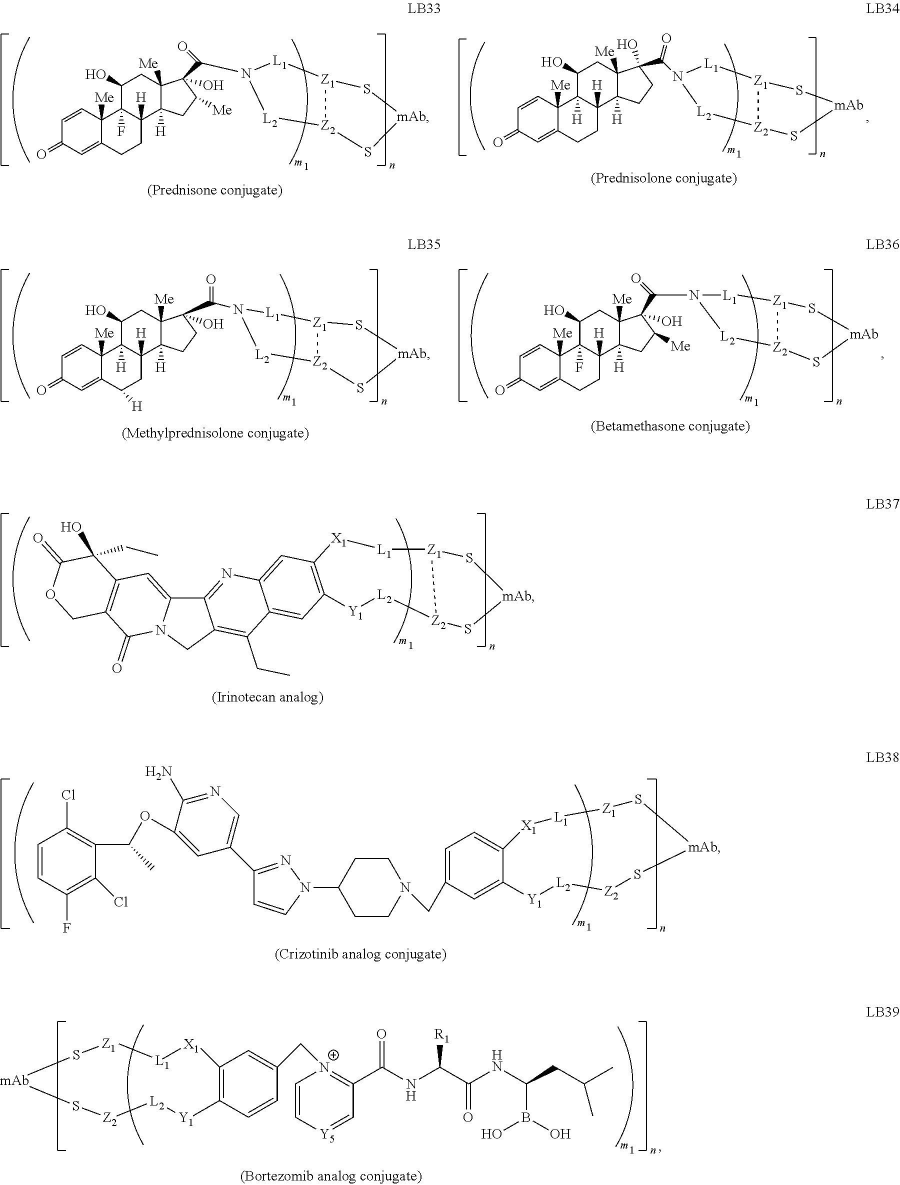

wherein Y₅, is N, CH, C(Cl), C(CH₃), or C(COOR₁); R₁ is H, C₁-C₆ Alkyl, C₃-C₈ Ar;

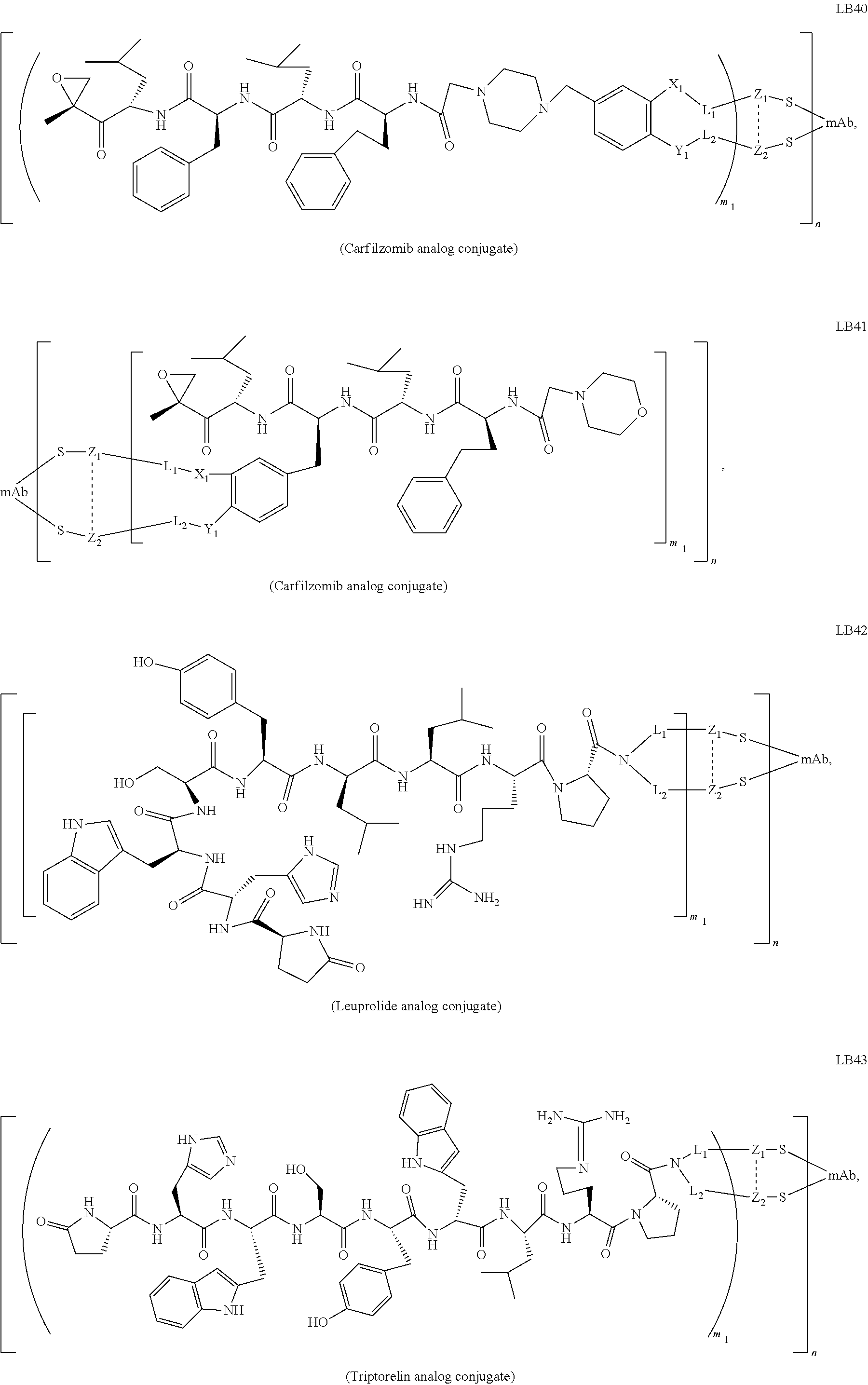

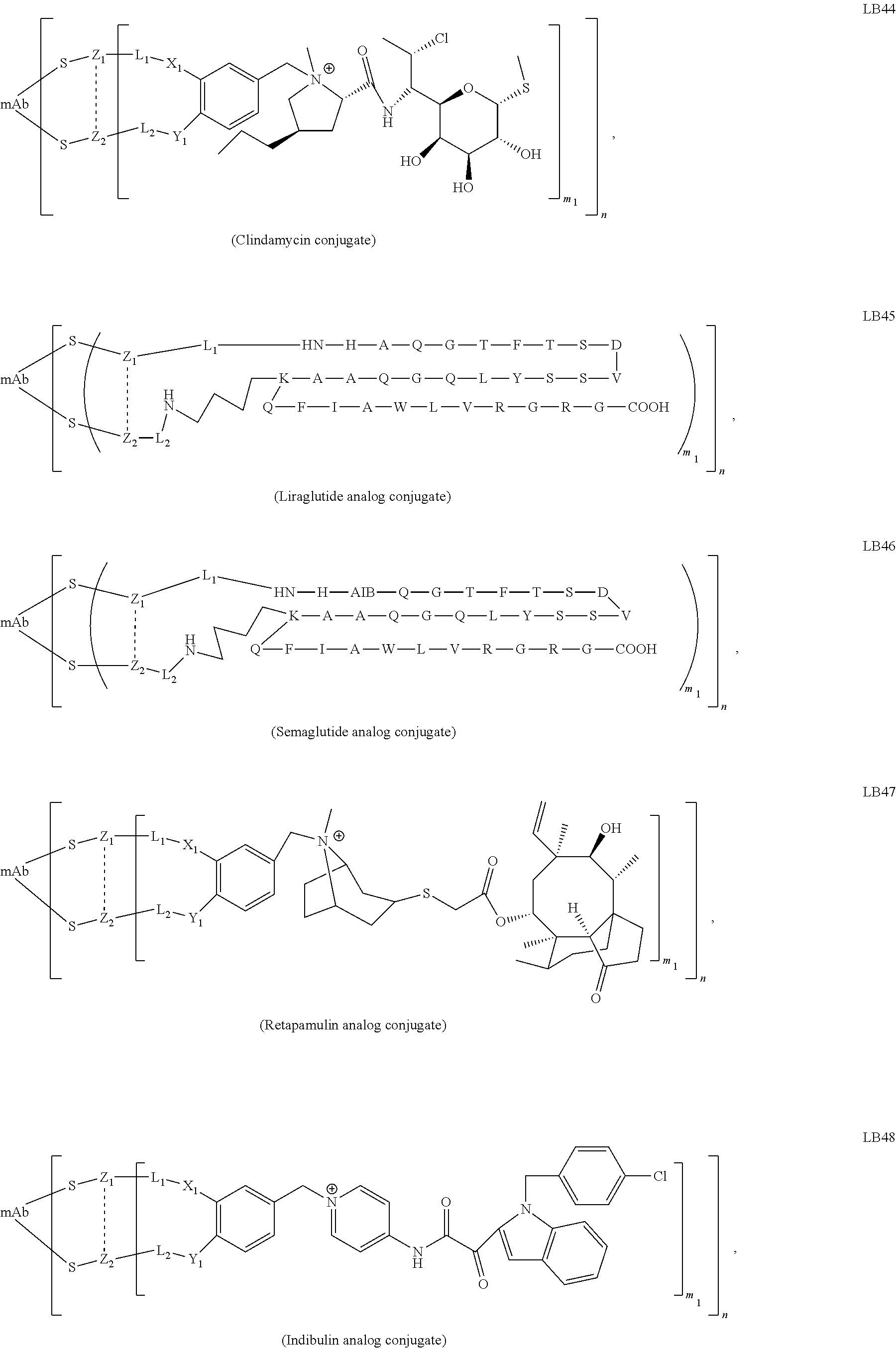

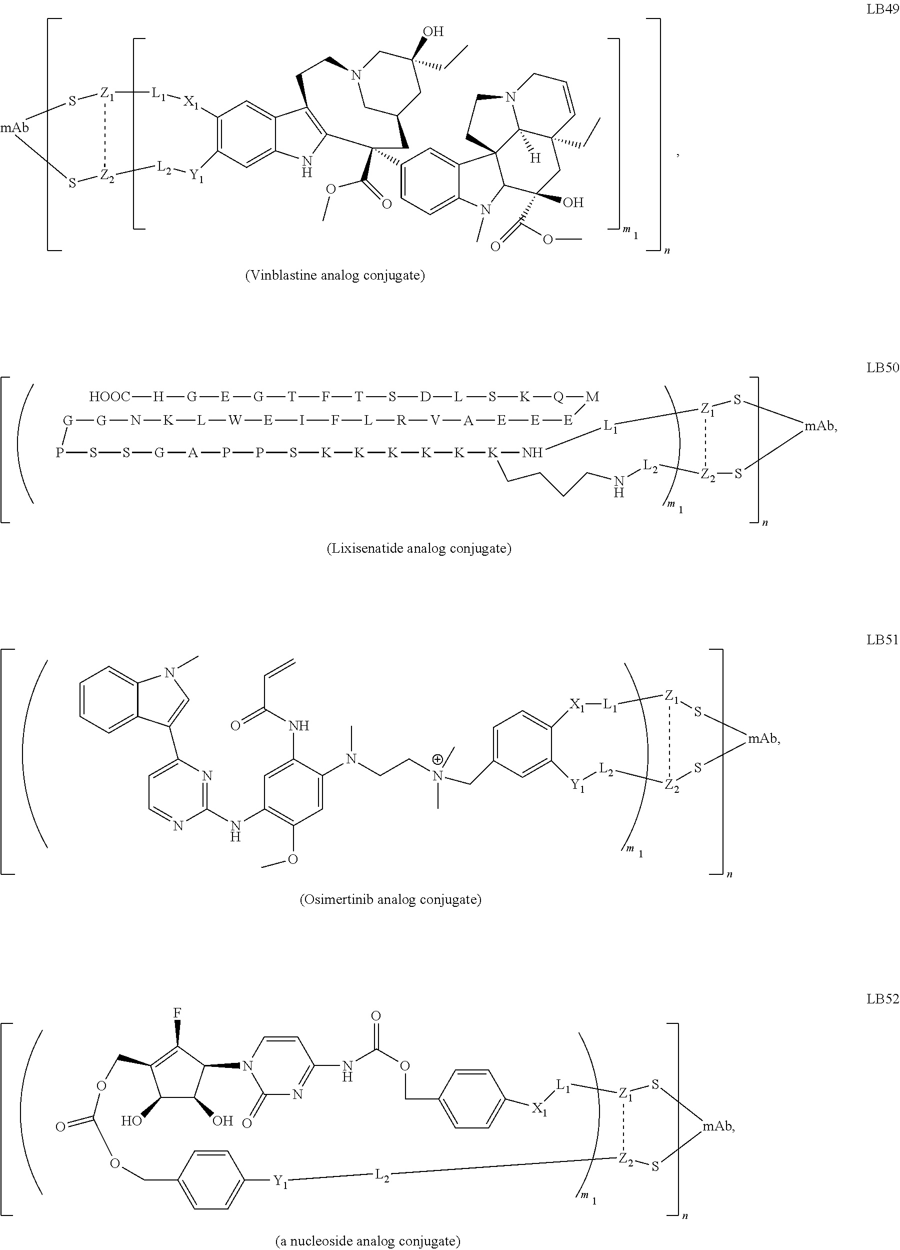

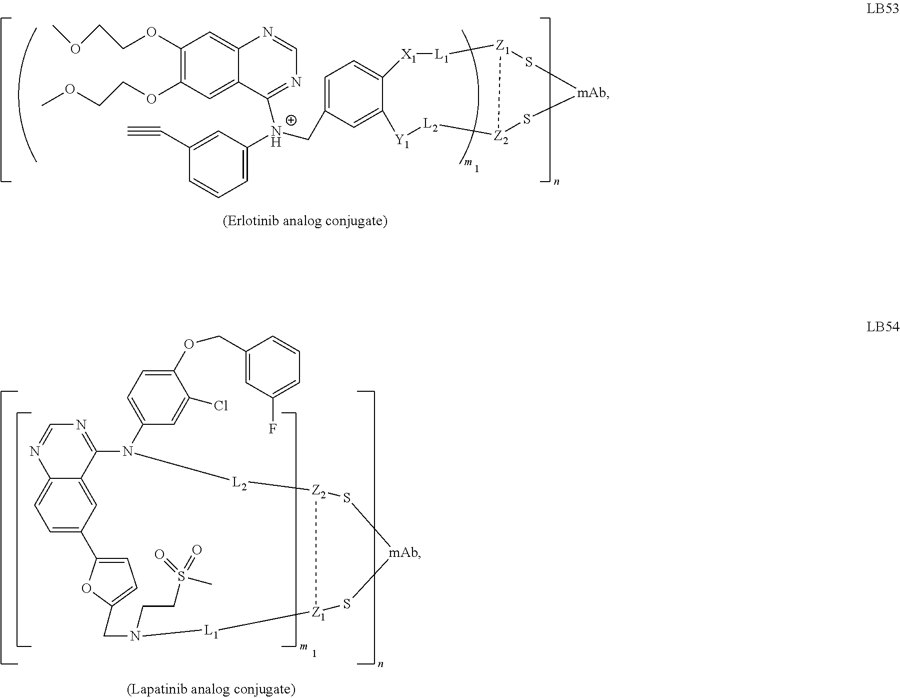

wherein “-----” is optionally either a single bond, or a double bond, or can optionally be absent; X₁, and Y₁ are independently O, NH, NHNH, NR₅, S, C(O)O, C(O)NH, OC(O)NH, OC(O)O, NHC(O)NH, NHC(O)S, OC(O)N(R₁), N(R₁)C(O)N(R₁), CH, C(O)NHNHC(O) and C(O)NR₁; mAb is antibody, preferably monoclonal antibody; n and m₁ are independently 1-20; L₁, L₂, R₁, R₁′, R₂, Z₁, and Z₂, are the same defined in Formula (I). X₃ is CH₂, O, NH, NHC(O), NHC(O)NH, C(O), OC(O), OC(O)(NR₃), R₁, NHR₁, NR₁, C(O)R₁ or absent; X₄ is H, CH₂, OH, O, C(O), C(O)NH, C(O)N(R₁), R₁, NHR₁, NR₁, C(O)R₁ or C(O)O; X₅ is H, CH₃, F, or Cl; M₁ and M₂ are independently H, Na, K, Ca, Mg, NH₄, NR₁R₂R₃; R₆ is 5′-deoxyadenosyl, Me, OH, or CN;

In yet another embodiment, one, two or more DNA, RNA, mRNA, small interfering RNA (siRNA), microRNA (miRNA), and PIWI interacting RNAs (piRNA) are preferred conjugated to a cell-binding molecule via a bis-linker of this patent. Small RNAs (siRNA, miRNA, piRNA) and long non-coding antisense RNAs are known responsible for epigenetic changes within cells (Goodchild, J (2011), Methods in molecular biology (Clifton, N.J.). 764: 1-15). DNA, RNA, mRNA, siRNA, miRNA or piRNA herein can be single or double strands with nucleotide units from 3 to 1 million and some of their nucleotide can be none natural (synthetic) forms, such as oligonucleotide with phosphorothioate linkage as example of Fomivirsen, or the nucleotides are linked with phosphorothioate linkages rather than the phosphodiester linkages of natural RNA and DNA, and the sugar parts are deoxyribose in the middle part of the molecule and 2′-O-methoxyethyl-modified ribose at the two ends as example Mipomersen, or oligonucleotide made with peptide nucleic acid (PNA), Morpholino, Phosphorothioate, Thiophosphoramidate, or with 2′-O-Methoxyethyl (MOE), 2′-O-Methyl, 2′-Fluoro, Locked Nucleic Acid (LNA), or Bicyclic Nucleic Acid (BNA) of ribose sugar, or nucleic acids are modified to remove the 2′-3′ carbon bond in the sugar ring (Whitehead, K. A.; et al (2011), Annual Review of Chemical and Biomolecular Engineering 2: 77-96; Bennett, C. F.; Swayze, E. E. (2010), Annu. Rev. Pharmacol. Toxicol. 50: 259-29). Preferably, oligonucleotide range in length is from approximately 8 to over 100 nucleotides. An example of the structure of the conjugates is displayed below:

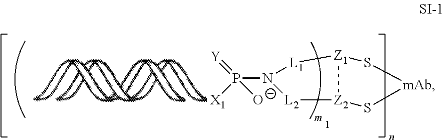

wherein mAb, m₁, n, X₁, L₁, L₂, Z₁, Z₂, “-----” are the same defined in Formula (I) or above; [symbol] is single or double strands of DNA, RNA, mRNA, siRNA, miRNA, or piRNA; Y is preferably O, S, NH or CH₂.

In yet another embodiment, IgG antibody conjugates conjugated with one, or two, or more differently function molecules or drugs are preferred to be conjugated specifically to a pair of thiols (through reduction of the disulfide bonds) between the light chain and heavy chain, the upper disulfide bonds between the two heavy chains, and the lower disulfide bonds between the two heavy chains as shown in the following structure, ST1, ST2, ST3, ST4, ST5, or ST6:

wherein Z₁, Z₂, X, Y, L₁, L₂, “-----”, m₁, and cytotoxic molecule are defined the same as X₁ in Formula (I) above;

In addition, the cytotoxic molecules and m₁ at different conjugation site of the cell-binding molecule can be different when the cytotoxic molecules containing the same or different bis-linkers are conjugated to a cell-binding molecule sequentially, or when different cytotoxic molecules containing the same or different bis-linkers are added stepwisely in a conjugation reaction mixture containing a cell-binding molecule.

Formulation and Application

The conjugates of the patent application are formulated to liquid, or suitable to be lyophilized and subsequently be reconstituted to a liquid formulation. A liquid formulation comprising 0.1 g/L˜300 g/L of concentration of the conjugate active ingredient for delivery to a patient without high levels of antibody aggregation may include one or more polyols (e.g. sugars), a buffering agent with pH 4.5 to 7.5, a surfactant (e.g. polysorbate 20 or 80), an antioxidant (e.g. ascorbic acid and/or methionine), a tonicity agent (e.g. mannitol, sorbitol or NaCl), chelating agents such as EDTA; metal complexes (e.g. Zn-protein complexes); biodegradable polymers such as polyesters; a preservative (e.g. benzyl alcohol) and/or a free amino acid.

Suitable buffering agents for use in the formulations include, but are not limited to, organic acid salts such as salts of citric acid, ascorbic acid, gluconic acid, carbonic acid, tartaric acid, succinic acid, acetic acid or phthalic acid; Tris, tromethamine (tris(hydroxymethyl)-aminomethane) hydrochloride, or phosphate buffer. In addition, amino acid components can also be used as buffering agent. Such amino acid component includes without limitation arginine, glycine, glycylglycine, and histidine. The arginine buffers include arginine acetate, arginine chloride, arginine phosphate, arginine sulfate, arginine succinate, etc. In one embodiment, the arginine buffer is arginine acetate. Examples of histidine buffers include histidine chloride-arginine chloride, histidine acetate-arginine acetate, histidine phosphate-arginine phosphate, histidine sulfate-arginine sulfate, histidine succinate-argine succinate, etc. The formulations of the buffers have a pH of 4.5 to pH 7.5, preferably from about 4.5 to about 6.5, more preferably from about 5.0 to about 6.2. In some embodiments, the concentration of the organic acid salts in the buffer is from about 10 mM to about 500 mM.

A “polyol” that may optionally be included in the formulation is a substance with multiple hydroxyl groups. Polyols can be used as stabilizing excipients and/or isotonicity agents in both liquid and lyophilized formulations. Polyols can protect biopharmaceuticals from both physical and chemical degradation pathways. Preferentially excluded co-solvents increase the effective surface tension of solvent at the protein interface whereby the most energetically favorable structural conformations are those with the smallest surface areas. Polyols include sugars (reducing and nonreducing sugars), sugar alcohols and sugar acids. A “reducing sugar” is one which contains a hemiacetal group that can reduce metal ions or react covalently with lysine and other amino groups in proteins and a “nonreducing sugar” is one which does not have these properties of a reducing sugar. Examples of reducing sugars are fructose, mannose, maltose, lactose, arabinose, xylose, ribose, rhamnose, galactose and glucose. Nonreducing sugars include sucrose, trehalose, sorbose, melezitose and raffinose. Sugar alcohols are selected from mannitol, xylitol, erythritol, maltitol, lactitol, erythritol, threitol, sorbitol and glycerol. Sugar acids include L-gluconate and its metallic salts thereof. Preferably, a nonreducing sugar: sucrose or trehalose at a concentration of about from 0.01% to 15% is chosen in the formulation, wherein trehalose being preferred over sucrose, because of the solution stability of trehalose.

A surfactant optionally in the formulations is selected from polysorbate (polysorbate 20, polysorbate 40, polysorbate 65, polysorbate 80, polysorbate 81, polysorbate 85 and the like); poloxamer (e.g. poloxamer 188, poly(ethylene oxide)-poly(propylene oxide), poloxamer 407 or polyethylene-polypropylene glycol and the like); Triton; sodium dodecyl sulfate (SDS); sodium laurel sulfate; sodium octyl glycoside; lauryl-, myristyl-, linoleyl-, or stearyl-sulfobetaine; lauryl-, myristyl-, linoleyl- or stearyl-sarcosine; linoleyl-, myristyl-, or cetyl-betaine; lauroamidopropyl-, cocamidopropyl-, linoleamidopropyl-, myristamidopropyl-, palmidopropyl-, or isostearamidopropyl-betaine (e.g. lauroamidopropyl); myristamidopropyl-, palmidopropyl-, or isostearamidopropyl-dimethylamine; sodium methyl cocoyl-, or disodium methyl oleyl-taurate; dodecyl betaine, dodecyl dimethylamine oxide, cocamidopropyl betaine and coco ampho glycinate; and the MONAQUAT™ series (e.g. isostearyl ethylimidonium ethosulfate); polyethyl glycol, polypropyl glycol, and copolymers of ethylene and propylene glycol (e.g. Pluronics, PF68 etc.); etc. Preferred surfactants are polyoxyethylene sorbitan fatty acid esters e.g. polysorbate 20, 40, 60 or 80 (Tween 20, 40, 60 or 80). The concentration of a surfactant is range from 0.0001% to about 1.0%. In certain embodiments, the surfactant concentration is from about 0.01% to about 0.1%. In one embodiment, the surfactant concentration is about 0.02%.

A “preservative” optionally in the formulations is a compound that essentially reduces bacterial action therein. Examples of potential preservatives include octadecyldimethylbenzyl ammonium chloride, hexamethonium chloride, benzalkonium chloride (a mixture of alkylbenzyldimethylammonium chlorides in which the alkyl groups are long-chain compounds), and benzethonium chloride. Other types of preservatives include aromatic alcohols such as phenol, butyl and benzyl alcohol, alkyl parabens such as methyl or propyl paraben, catechol, resorcinol, cyclohexanol, 3-pentanol, and m-cresol. The preservative is less than 5% in the formulation. Preferably 0.01% to 1%. In one embodiment, the preservative herein is benzyl alcohol.

Suitable free amino acids optionally for use in the formulation, but are not limited to, are arginine, lysine, histidine, ornithine, isoleucine, leucine, alanine, glycine glutamic acid or aspartic acid. The inclusion of a basic amino acid is preferred i.e. arginine, lysine and/or histidine. If a composition includes histidine then this may act both as a buffering agent and a free amino acid, but when a histidine buffer is used it is typical to include a non-histidine free amino acid e.g. to include histidine buffer and lysine. An amino acid may be present in its D- and/or L-form, but the L-form is typical. The amino acid may be present as any suitable salt e.g. a hydrochloride salt, such as arginine-HCl. The concentration of an amino acid is range from 0.0001% to about 15.0%. Preferably 0.01% to 5%.

The formulations can optionally comprise methionine or ascorbic acid as an antioxidant at a concentration of about from 0.01 mg/ml to 5 mg/ml; The formulations can optionally comprise chelating agent, e.g., EDTA, EGTA, etc., at a concentration of about from 0.01 mM to 2 mM.

The final formulation can be adjusted to the preferred pH with an adjust agent (e.g. an acid, such as HCl, H₂SO₄, acetic acid, H₃PO₄, citric acid, etc., or a base, such as NaOH, KOH, NH₃OH, ethanolamine, diethanolamine or triethanol amine, sodium phosphate, potassium phosphate, trisodium citrate, tromethamine, etc.) and the formulation should be controlled “isotonic” which is meant that the formulation of interest has essentially the same osmotic pressure as human blood. Isotonic formulations will generally have an osmotic pressure from about 250 to 350 mOsm. Isotonicity can be measured using a vapor pressure or ice-freezing type osmometer, for example.

Other excipients which may be useful in either a liquid or lyophilized formulation of the patent application include, for example, fucose, cellobiose, maltotriose, melibiose, octulose, ribose, xylitol, arginine, histidine, glycine, alanine, methionine, glutamic acid, lysine, imidazole, glycylglycine, mannosylglycerate, Triton X-100, Pluoronic F-127, cellulose, cyclodextrin, dextran (10, 40 and/or 70 kD), polydextrose, maltodextrin, ficoll, gelatin, hydroxypropylmeth, sodium phosphate, potassium phosphate, ZnCl₂, zinc, zinc oxide, sodium citrate, trisodium citrate, tromethamine, copper, fibronectin, heparin, human serum albumin, protamine, glycerin, glycerol, EDTA, metacresol, benzyl alcohol, phenol, polyhydric alcohols, or polyalcohols, hydrogenated forms of carbohydrate having a carbonyl group reduced to a primary or secondary hydroxyl group.

Other contemplated excipients, which may be utilized in the aqueous pharmaceutical compositions of the patent application include, for example, flavoring agents, antimicrobial agents, sweeteners, antioxidants, antistatic agents, lipids such as phospholipids or fatty acids, steroids such as cholesterol, protein excipients such as serum albumin (human serum albumin), recombinant human albumin, gelatin, casein, salt-forming counterions such sodium and the like. These and additional known pharmaceutical excipients and/or additives suitable for use in the formulations of the invention are known in the art, e.g., as listed in “The Handbook of Pharmaceutical Excipients, 4^{th} edition, Rowe et al., Eds., American Pharmaceuticals Association (2003); and Remington: the Science and Practice of Pharmacy, 21^{th} edition, Gennaro, Ed., Lippincott Williams & Wilkins (2005).

In a further embodiment, the invention provides a method for preparing a formulation comprising the steps of: (a) lyophilizing the formulation comprising the conjugates, excipients, and a buffer system to a powder; and (b) reconstituting the lyophilized mixture of step (a) in a reconstitution medium such that the reconstituted formulation is stable. The formulation of step (a) may further comprise a stabilizer and one or more excipients selected from a group comprising bulking agent, salt, surfactant and preservative as hereinabove described. As reconstitution media several diluted organic acids or water, i.e. sterile water, bacteriostatic water for injection (BWFI) or may be used. The reconstitution medium may be selected from water, i.e. sterile water, bacteriostatic water for injection (BWFI) or the group consisting of acetic acid, propionic acid, succinic acid, sodium chloride, magnesium chloride, acidic solution of sodium chloride, acidic solution of magnesium chloride and acidic solution of arginine, in an amount from about 10 to about 250 mM.

A liquid pharmaceutical formulation of the conjugates of the patent application should exhibit a variety of pre-defined characteristics. One of the major concerns in liquid drug products is stability, as proteins/antibodies tend to form soluble and insoluble aggregates during manufacturing and storage. In addition, various chemical reactions can occur in solution (deamidation, oxidation, clipping, isomerization etc.) leading to an increase in degradation product levels and/or loss of bioactivity. Preferably, a conjugate in either liquid or loyphilizate formulation should exhibit a shelf life of more than 18 months at 25° C. More preferred a conjugate in either liquid or loyphilizate formulation should exhibit a shelf life of more than 24 months at 25° C. Most preferred liquid formulation should exhibit a shelf life of about 24 to 36 months at 2-8° C. and the loyphilizate formulation should exhibit a shelf life of about preferably up to 60 months at 2-8° C. Both liquid and loyphilizate formulations should exhibit a shelf life for at least two years at −20° C., or −70° C.

In certain embodiments, the formulation is stable following freezing (e. g., −20° C., or −70° C.) and thawing of the formulation, for example following 1, 2 or 3 cycles of freezing and thawing. Stability can be evaluated qualitatively and/or quantitatively in a variety of different ways, including evaluation of drug/antibody (protein) ratio and aggregate formation (for example using UV, size exclusion chromatography, by measuring turbidity, and/or by visual inspection); by assessing charge heterogeneity using cation exchange chromatography, image capillary isoelectric focusing (icIEF) or capillary zone electrophoresis; amino-terminal or carboxy-terminal sequence analysis; mass spectrometric analysis, or matrix-assisted laser desorption ionization/time-of-flight mass spectrometry (MALDI/TOF MS), or HPLC-MS/MS; SDS-PAGE analysis to compare reduced and intact antibody; peptide map (for example tryptic or LYS-C) analysis; evaluating biological activity or antigen binding function of the antibody; etc. Instability may involve any one or more of: aggregation, deamidation (e.g. Asn deamidation), oxidation (e.g. Met oxidation), isomerization (e.g. Asp isomeriation), clipping/hydrolysis/fragmentation (e.g. hinge region fragmentation), succinimide formation, unpaired cysteine(s), N-terminal extension, C-terminal processing, glycosylation differences, etc.

A stable conjugate should also “retains its biological activity” in a pharmaceutical formulation, if the biological activity of the conjugate at a given time, e. g. 12 month, within about 20%, preferably about 10% (within the errors of the assay) of the biological activity exhibited at the time the pharmaceutical formulation was prepared as determined in an antigen binding assay, and/or in vitro, cytotoxic assay, for example.

A pharmaceutical container or vessel is used to hold the pharmaceutical formulation of any of conjugates of the patent application. The vessel is a vial, bottle, pre-filled syringe, or pre-filled auto-injector syringe.

For clinical in vivo use, the conjugate via the bis-linkage of the invention will be supplied as solutions or as a lyophilized solid that can be redissolved in sterile water for injection. Examples of suitable protocols of conjugate administration are as follows. Conjugates are given daily, weekly, biweekly, triweekly, once every four weeks or monthly for 8˜54 weeks as an i.v. bolus. Bolus doses are given in 50 to 1000 ml of normal saline to which human serum albumin (e.g. 0.5 to 1 mL of a concentrated solution of human serum albumin, 100 mg/mL) can optionally be added. Dosages will be about 50 μg to 20 mg/kg of body weight per week, i.v. (range of 10 μg to 200 mg/kg per injection). 4˜54 weeks after treatment, the patient may receive a second course of treatment. Specific clinical protocols with regard to route of administration, excipients, diluents, dosages, times, etc., can be determined by the skilled clinicians.

Examples of medical conditions that can be treated according to the in vivo or ex vivo methods of killing selected cell populations include malignancy of any types of cancer, autoimmune diseases, graft rejections, and infections (viral, bacterial or parasite).

The amount of a conjugate which is required to achieve the desired biological effect, will vary depending upon a number of factors, including the chemical characteristics, the potency, and the bioavailability of the conjugates, the type of disease, the species to which the patient belongs, the diseased state of the patient, the route of administration, all factors which dictate the required dose amounts, delivery and regimen to be administered.

In general terms, the conjugates via the bis-linkers of this invention may be provided in an aqueous physiological buffer solution containing 0.1 to 10% w/v conjugates for parenteral administration. Typical dose ranges are from 1 μg/kg to 0.1 g/kg of body weight daily; weekly, biweekly, triweekly, or monthly, a preferred dose range is from 0.01 mg/kg to 20 mg/kg of body weight weekly, biweekly, triweekly, or monthly, an equivalent dose in a human. The preferred dosage of drug to be administered is likely to depend on such variables as the type and extent of progression of the disease or disorder, the overall health status of the particular patient, the relative biological efficacy of the compound selected, the formulation of the compound, the route of administration (intravenous, intramuscular, or other), the pharmacokinetic properties of the conjugates by the chosen delivery route, and the speed (bolus or continuous infusion) and schedule of administrations (number of repetitions in a given period of time).

The conjugates via the linkers of the present invention are also capable of being administered in unit dose forms, wherein the term “unit dose” means a single dose which is capable of being administered to a patient, and which can be readily handled and packaged, remaining as a physically and chemically stable unit dose comprising either the active conjugate itself, or as a pharmaceutically acceptable composition, as described hereinafter. As such, typical total daily/weekly/biweekly/monthly dose ranges are from 0.01 to 100 mg/kg of body weight. By way of general guidance, unit doses for humans range from 1 mg to 3000 mg per day, or per week, per two weeks (biweekly), triweekly, or per month. Preferably the unit dose range is from 1 to 500 mg administered one to four times a month, and even more preferably from 1 mg to 100 mg, once a week, or once biweekly, or once triweekly. Conjugates provided herein can be formulated into pharmaceutical compositions by admixture with one or more pharmaceutically acceptable excipients. Such unit dose compositions may be prepared for use by oral administration, particularly in the form of tablets, simple capsules or soft gel capsules; or intranasal, particularly in the form of powders, nasal drops, or aerosols; or dermally, for example, topically in ointments, creams, lotions, gels or sprays, or via transdermal patches.

In yet another embodiment, a pharmaceutical composition comprising a therapeutically effective amount of the conjugate of Formula (II) or any conjugates described through the present patent can be administered concurrently with the other therapeutic agents such as the chemotherapeutic agent, the radiation therapy, immunotherapy agents, autoimmune disorder agents, anti-infectious agents or the other conjugates for synergistically effective treatment or prevention of a cancer, or an autoimmune disease, or an infectious disease. The synergistic agents are preferably selected from one or several of the following drugs: Abatacept (Orencia), Abiraterone acetate (Zytiga®), Abraxane, Acetaminophen/hydrocodone, Adalimumab, afatinib dimaleate (Gilotrif®), Alectinib (Alecensa), alemtuzumab (Campath®), Alitretinoin (Panretin®), ado-trastuzumab emtansine (Kadcyla™), Amphetamine mixed salts (Amphetamine/dextroamphetamine, or Adderall XR), anastrozole (Arimidex®), Aripiprazole, Atazanavir, Atezolizumab (Tecentriq, MPDL3280A), Atorvastatin, axitinib (Inlyta®), AZD9291, belinostat (Beleodaq™), Bevacizumab (Avastin®), Bortezomib (PS-341; Velcade, Neomib, Bortecad), Cabazitaxel (Jevtana®), Cabozantinib (Cometriq™), bexarotene (Targrtin®), Blinatumomab (Blincyto™), Bortezomib (Velcade®), bosutinib (Bosulif®), brentuximab vedotin (Adcetris®), Budesonide, Budesonide/formoterol, Buprenorphine, Capecitabine, carfilzomib (Kyprolis®), Celecoxib, ceritinib (LDK378/Zykadia), Cetuximab (Erbitux®), Ciclosporin, Cinacalcet, Crizotinib (Xalkori®), Cobimetinib (Cotellic), Dabigatran, dabrafenib (Tafinlar®), Daratumumab (Darzalex), Darbepoetin alfa, Darunavir, imatinib mesylate (Gleevec®), dasatinib (Sprycel®), denileukin diftitox (Ontak®), Denosumab (Xgeva®), Depakote, Dexamethasone, Dexlansoprazole, Dexmethylphenidate, Dinutuximab (Unituxin™), Doxycycline, Duloxetine, Durvalumab (MEDI4736), Elotuzumab (Empliciti), Emtricitabine/Rilpivirine/Tenofovir disoproxil fumarate, Emtricitbine/tenofovir/efavirenz, Enoxaparin, Enzalutamide (Xtandi®), Epoetin alfa, erlotinib (Tarceva®), Esomeprazole, Eszopiclone, Etanercept, Everolimus (Afinitor®), exemestane (Aromasin®), everolimus (Afinitor®), Ezetimibe, Ezetimibe/simvastatin, Fenofibrate, Filgrastim, fingolimod, Fluticasone propionate, Fluticasone/salmeterol, fulvestrant (Faslodex®), gefitinib (Iressa®), Glatiramer, Goserelin acetate (Zoladex), Icotinib, Imatinib (Gleevec), Ibritumomab tiuxetan (Zevalin®), ibrutinib (Imbruvica™), idelalisib (Zydelig®), Infliximab, iniparib, Insulin aspart, Insulin detemir, Insulin glargine, Insulin lispro, Interferon beta 1a, Interferon beta 1b, lapatinib (Tykerb®), Ipilimumab (Yervoy®), Ipratropium bromide/salbutamol, Ixazomib (Ninlaro), Lanreotide acetate (Somatuline® Depot), Lenaliomide (Revlimid®), Lenvatinib (Lenvima™), letrozole (Femara®), Levothyroxine, Levothyroxine, Lidocaine, Linezolid, Liraglutide, Lisdexamfetamine, MEDI4736 (AstraZeneca, Celgene), Memantine, Methylphenidate, Metoprolol, Modafinil, Mometasone, Necitumumab (Portrazza), Nilotinib (Tasigna®), niraparib, Nivolumab (Opdivo®), ofatumumab (Arzerra®), obinutuzumab (Gazyva™), Olaparib (Lynparza™), Olmesartan, Olmesartan/hydrochlorothiazide, Omalizumab, Omega-3 fatty acid ethyl esters, Oseltamivir, Osimertinib (or mereletinib, Tagrisso), Oxycodone, Palbociclib (Ibrance®), Palivizumab, panitumumab (Vectibix®), panobinostat (Farydak®), pazopanib (Votrient®), Pembrolizumab (Keytruda®), Pemetrexed (Alimta), pertuzumab (Perjeta™), Pneumococcal conjugate vaccine, pomalidomide (Pomalyst®), Pregabalin, Propranolol, Quetiapine, Rabeprazole, radium 223 chloride (Xofigo®), Raloxifene, Raltegravir, Ramucirumab (Cyramza®), Ranibizumab, regorafenib (Stivarga®), Rituximab (Rituxan®), Rivaroxaban, romidepsin (Istodax®), Rosuvastatin, ruxolitinib phosphate (Jakafi™), Salbutamol, Sevelamer, Sildenafil, siltuximab (Sylvant™), Sitagliptin, Sitagliptin/metformin, Solifenacin, Sonidegib (LDE225, Odomzo), Sorafenib (Nexavar®), Sunitinib (Sutent®), Tadalafil, tamoxifen, Telaprevir, talazoparib, temsirolimus (Torisel®), Tenofovir/emtricitabine, Testosterone gel, Thalidomide (Immunoprin, Talidex), Tiotropium bromide, toremifene (Fareston®), trametinib (Mekinist®), Trastuzumab, Trabectedin (ecteinascidin 743, Yondelis), Trifluridine/tipiracil (Lonsurf, TAS-102), Tretinoin (Vesanoid®), Ustekinumab, Valsartan, veliparib, vandetanib (Caprelsa®), Vemurafenib (Zelboraf®), Venetoclax (Venclexta), vorinostat (Zolinza®), ziv-aflibercept (Zaltrap®), Zostavax., and their analogs, derivatives, pharmaceutically acceptable salts, carriers, diluents, or excipients thereof, or a combination above thereof.

The drugs/cytotoxic agents used for conjugation via a bridge linker of the present patent can be any analogues and/or derivatives of drugs/molecules described in the present patent. One skilled in the art of drugs/cytotoxic agents will readily understand that each of the drugs/cytotoxic agents described herein can be modified in such a manner that the resulting compound still retains the specificity and/or activity of the starting compound. The skilled artisan will also understand that many of these compounds can be used in place of the drugs/cytotoxic agents described herein. Thus, the drugs/cytotoxic agents of the present invention include analogues and derivatives of the compounds described herein.

All references cited herein and in the examples that follow are expressly incorporated by reference in their entireties.

### EXAMPLES

The invention is further described in the following examples, which are not intended to limit the scope of the invention. Cell lines described in the following examples were maintained in culture according to the conditions specified by the American Type Culture Collection (ATCC) or Deutsche Sammlung von Mikroorganismen und Zellkulturen GmbH, Braunschweig, Germany (DMSZ), or The Shanghai Cell Culture Institute of Chinese Acadmy of Science, unless otherwise specified. Cell culture reagents were obtained from Invitrogen Corp., unless otherwise specified. All anhydrous solvents were commercially obtained and stored in Sure-seal bottles under nitrogen. All other reagents and solvents were purchased as the highest grade available and used without further purification. The preparative HPLC separations were performed with Varain PreStar HPLC. NMR spectra were recorded on Varian Mercury 400 MHz Instrument. Chemical shifts (.delta.) are reported in parts per million (ppm) referenced to tetramethylsilane at 0.00 and coupling constants (J) are reported in Hz. The mass spectral data were acquired on a Waters Xevo QTOF mass spectrum equipped with Waters Acquity UPLC separations module and Acquity TUV detector.

### Example 1. Synthesis of di-tert-butyl 1,2-bis(2-(tert-butoxy)-2-oxoethyl)hydrazine-1,2-dicarboxylate

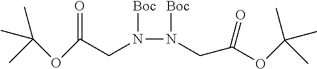 [SMILES: CC(C)(C)OC(=O)CN(C(=O)OC(C)(C)C)N(CC(=O)OC(C)(C)C)C(=O)OC(C)(C)C]

To di-tert-butyl hydrazine-1,2-dicarboxylate (8.01 g, 34.4 mmol) in DMF (150 ml) was added NaH (60% in oil, 2.76 g, 68.8 mmol). After stirred at RT for 30 min, tert-butyl 2-bromoacetate (14.01 g, 72.1 mmol) was added. The mixture was stirred overnight, quenched with addition of methanol (3 ml), concentrated, diluted with EtOAc (100 ml) and water (100 ml), separated, and the aqueous layer was extracted with EtOAc (2×50 ml). The organic layers were combined, dried over MgSO₄, filtered, evaporated, and purified by SiO₂ column chromatography (EtOAc/Hexane 1:5 to 1:3) to afforded the title compound (12.98 g, 82% yield) as a colorless oil. MS ESI m/z calcd for C₂₂H₄₁N₂O₈ [M+H]⁺ 461.28. found 461.40.

### Example 2. Synthesis of 2,2′-(hydrazine-1,2-diyl)diacetic Acid

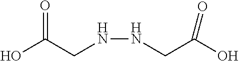

Di-tert-butyl 1,2-bis(2-(tert-butoxy)-2-oxoethyl)hydrazine-1,2-dicarboxylate (6.51 g, 14.14 mmol) in 1,4-dioxane (40 ml) was added HCl (12 M, 10 ml). The mixture was stirred for 30 min, diluted with dioxane (20 ml) and toluene (40 ml), evaporated and co-evaporated with dioxane (20 ml) and toluene (40 ml) to dryness to afford the crude title product for the next step without further production (2.15 g, 103% yield, ˜93% pure). MS ESI m/z calcd for C₄H₉N₂O₄ [M+H]⁺ 149.05. found 149.40.

### Example 3. Synthesis of 2,2′-(1,2-bis((benzyloxy)carbonyl)hydrazine-1,2-diyl)diacetic Acid

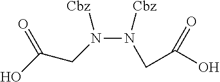 [SMILES: O=C(O)CN(C(=O)OCc1ccccc1)N(CC(=O)O)C(=O)OCc1ccccc1]

To a solution of 2,2′-(hydrazine-1,2-diyl)diacetic acid (1.10 g, 7.43 mmol) in the mixture of THF (200 ml) and NaH₂PO₄ (0.1 M, 250 ml, pH 8.0) was added benzyl carbonochloridate (5.01 g, 29.47 mmol) in 4 portions in 2 h. The mixture was stirred for another 6 h, concentrated and purified on SiO₂ column eluted with H₂O/CH₃CN (1:9) containing 1% formic acid to afford the title compound (2.26 g, 73% yield, ˜95% pure). MS ESI m/z calcd for C₂₀H₂₁N₂O₈ [M+H]⁺ 417.12. found 417.40.

### Example 4. Synthesis of dibenzyl 1,2-bis(2-chloro-2-oxoethyl)hydrazine-1,2-dicarboxylate

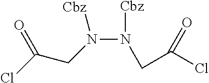 [SMILES: O=C(Cl)CN(C(=O)OCc1ccccc1)N(CC(=O)Cl)C(=O)OCc1ccccc1]

2,2′-(1,2-bis((benzyloxy)carbonyl)hydrazine-1,2-diyl)diacetic acid (350 mg, 0.841 mmol) in dichloroethane (30 ml) was added (COCl)₂ (905 mg, 7.13 mmol), followed by addition of 0.030 ml of DMF. After stirred at RT for 2 h, the mixture was diluted with toluene, concentrated and co-evaporated with dichloroethane (2×20 ml) and toluene (2×15 ml) to dryness to afford the title crude product (which is not stable) for the next step without further purification (365 mg, 96% yield). MS ESI m/z calcd for C₂₀H1₉Cl₂N₂O₆ [M+H]⁺ 453.05. found 453.50.

### Example 5. Synthesis of di-tert-butyl 1,2-bis(2-(tert-butoxy)-2-oxoethyl)hydrazine-1,2-dicarboxylate

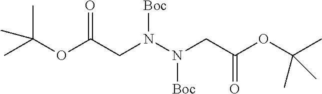 [SMILES: CC(C)(C)OC(=O)CN(C(=O)OC(C)(C)C)N(CC(=O)OC(C)(C)C)C(=O)OC(C)(C)C]

To a suspension of NaH (0.259 g, 6.48 mmol, 3.0 eq.) in anhydrous DMF (2 mL) at room temperature was added di-tert-butyl hydrazine-1,2-dicarboxylate (0.50 g, 2.16 mmol, 1.0 eq.) in anhydrous DMF (8 mL) in 10 minutes under nitrogen. The mixture was stirred at room temperature for 10 minutes and then cooled to 0° C. To which tert-butyl 2-bromoacetate (1.4 mL, 8.61 mmol, 4.0 eq.) was added dropwise. The resulting mixture was allowed to warm to room temperature and stirred overnight. Saturated ammonium chloride solution (100 mL) was added. The organic layer was separated and the aqueous layer was extracted with EtOAc (3×50 mL). The combined organic solution was washed with water and brine, dried over anhydrous Na₂SO₄, concentrated and purified by SiO₂ column chromatography (10:1 hexanes/EtOAc) to give the title compound as a colourless oil (0.94 g, 99.6% yield). ESI MS m/z [M+Na]⁺483.4.

### Example 6. Synthesis of compound 2,2′-(hydrazine-1,2-diyl)diacetic Acid

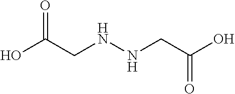

To a solution of di-tert-butyl 1,2-bis(2-(tert-butoxy)-2-oxoethyl)hydrazine-1,2-dicarboxylate (0.94 g, 2.04 mmol) in DCM (4 mL) at 0° C. was added TFA (4 mL). The reaction was stirred for 30 minutes and then warmed to room temperature and stirred overnight. The mixture was concentrated, diluted with DCM, and concentrated. This operation was repeated for three times to give a white solid. Trituration with DCM and a white solid was collected by filtration (0.232 g, 76.8% yield). ESI MS m/z [M+H]⁺ 149.2.

### Example 7. Synthesis of 2,2′-(1,2-bis(2-chloroacetyl)hydrazine-1,2-diyl)diacetic Acid

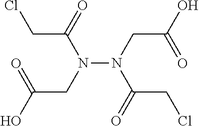 [SMILES: O=C(O)CN(C(=O)CCl)N(CC(=O)O)C(=O)CCl]

To a solution of 2,2′-(hydrazine-1,2-diyl)diacetic acid (0.232 g, 1.57 mmol, 1.0 eq.) in anhydrous THF (10 mL) at 0° C. was added 2-chloroacetyl chloride (0.38 mL, 4.70 mmol, 3.0 eq.) in 10 minutes. The reaction was warmed to room temperature and stirred overnight and concentrated. The residue was co-evaporated with THF for three times to give a white solid (0.472 g, theoretical yield). ESI MS m/z [M+H]⁺ 301.1.

### Example 8. Synthesis of tert-butyl 2,8-dioxo-1,5-oxazocane-5-carboxylate

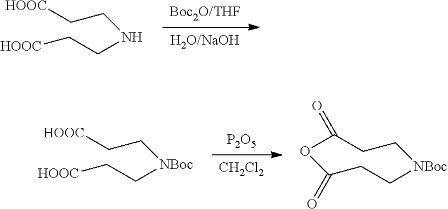

To a solution of 3,3′-azanediyldipropanoic acid (10.00 g, 62.08 mmol) in 1.0 M NaOH (300 ml) at 4° C. was added di-tert-butyl dicarbonate (22.10 g, 101.3 mmol) in 200 ml THF in 1 h. After addition, the mixture was kept to stirring for 2 h at 4° C. The mixture was carefully acidified to pH ˜4 with 0.2 M H₃PO₄, concentrated in vacuo, extracted with CH2Cl2, dried over Na2SO4, evaporated and purified with flash SiO₂ chromatography eluted with AcOH/MeOH/CH₂Cl₂ (0.01:1:5) to afford 3,3′-((tert-butoxycarbonyl)azanediyl)dipropanoic acid (13.62 g, 84% yield). ESI MS m/z C₁₁H₁₉NO₆ [M+H]⁺, cacld. 262.27. found 262.40.

To a solution of 3,3′-((tert-butoxycarbonyl)azanediyl)dipropanoic acid (8.0 g, 30.6 mmol) in CH₂Cl₂ (500 ml) at 0° C. was added phosphorus pentoxide (8.70 g, 61.30 mmol). The mixture was stirred at 0° C. for 2 h and then r.t. for 1 h, filtered through short SiO₂ column, and rinsed the column with EtOAc/CH₂Cl₂ (1:6). The filtrate was concentrated and triturated with EtOAc/hexane to afford the title compound (5.64 g, 74% yield). ESI MS m/z C₁₁H₁₇NO₅ [M+H]⁺, cacld. 244.11. found 244.30.

### Example 9. Synthesis of tert-Butyl 3-((benzyloxy)amino)propanoate

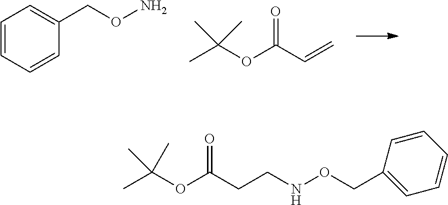

O-benzylhydroxylamine hydrochloride salt (10.0 g, 62.7 mmol) in THF (100 ml) was added Et₃N (15 ml) and tert-butyl acrylate (12.1 g, 94.5 mmol). The mixture was refluxed for overnight, concentrated and purified on SiO₂ column eluted with EtOAc/Hexane (1:4) to afford the title compound 3 (13.08 g, 83% yield). 1H NMR (CDCl₃) 7.49˜7.25 (m, 5H), 4.75 (s, 2H), 3.20 (t, J=6.4 Hz, 2H), 2.54 (t, J=6.4 Hz, 2H), 1.49 (s, 9H); ESI MS m/z+ C₁₄H₂₁NNaO₃ (M+Na), cacld. 274.15. found 274.20.

### Example 10. Synthesis of tert-Butyl 3-(hydroxyamino)propanoate

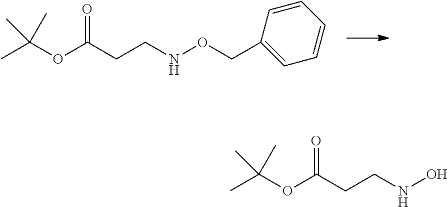 [SMILES: CC(C)(C)OC(=O)CCNOCc1ccccc1 ; CC(C)(C)OC(=O)CCNO]

tert-Butyl 3-((benzyloxy)amino)propanoate (13.0 g, 51.76 mmol) in methanol (100 ml) was added Pd/C (0.85 g, 10% Pd, 50% wet) in a hydrogenation vessel. After the system was evacuated under vacuum and placed under 2 atm of hydrogen gas, the reaction mixture was stirred overnight at room temperature. The crude reaction was passed through a short pad of Celite rinsing with ethanol, concentrated and purified on SiO₂ column eluted with MeOH/DCM (1:10-1:5) to afford the title compound (7.25 g, 87% yield). 1H NMR (CDCl₃) 3.22 (t, J=6.4 Hz, 2H), 2.55 (t, J=6.4 Hz, 2H), 1.49 (s, 9H); ESI MS m/z+ C₇H₁₅NNaO₃ (M+Na), cacld. 184.10, found 184.30.

### Example 11. Synthesis of tert-Butyl 3-((tosyloxy)amino)propanoate

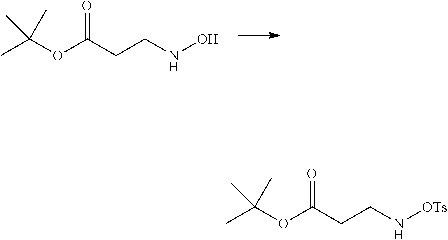 [SMILES: CC(C)(C)OC(=O)CCNO ; Cc1ccc(S(=O)(=O)ONCCC(=O)OC(C)(C)C)cc1]

Tert-butyl 3-(hydroxyamino)propanoate (5.10 g, 31.65 mmol) in the mixture of DCM (50 ml) and pyridine (20 ml) was added tosylate chloride (12.05 g, 63.42) at 4° C. After addition, the mixture was stirred at room temperature overnight, concentrated and purified on SiO₂ column eluted with EtOAc/DCM (1:10˜1:6) to afford the title compound (8.58 g, 86% yield). 1H NMR (CDCl₃) 7.81 (s, 2H), 7.46 (s, 2H), 3.22 (t, J=6.4 Hz, 2H), 2.55 (t, J=6.4 Hz, 2H), 2.41 (s, 3H), 1.49 (s, 9H); ESI MS m/z+ C₁₄H₂₁NNaO₅S (M+Na), cacld. 338.11. found 338.30.

### Example 12. Synthesis of di-tert-Butyl 3,3′-(hydrazine-1,2-diyl)dipropanoate

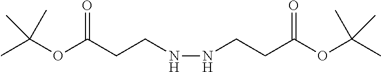 [SMILES: CC(C)(C)OC(=O)CCNNCCC(=O)OC(C)(C)C]

Tert-butyl 3-aminopropanoate (3.05 g, 21.01 mmol) in THF (80 ml) was added tert-Butyl 3-((tosyloxy)amino)propanoate (5.10 g, 16.18 mmol). The mixture was stirred at room temperature for 1 h and then 45° C. for 6 h. The mixture was concentrated and purified on SiO₂ column eluted with CH₃OH/DCM/Et₃N (1:12:0.01˜1:8:0.01) to afford the title compound (2.89 g, 62% yield). ESI MS m/z+ C₁₄H₂₈N₂NaO₄ (M+Na), cacld. 311.20. found 311.40.

### Example 13. Synthesis of di-tert-Butyl 3,3′-(1,2-bis(3-(2,5-dioxo-2,5-dihydro-1H-pyrrol-1-yl)propanoyl)hydrazine-1,2-diyl)dipropanoate

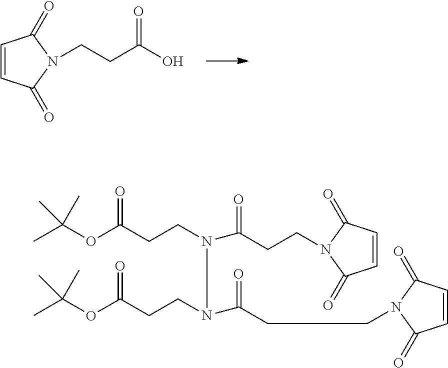 [SMILES: O=C(O)CCN1C(=O)C=CC1=O ; CC(C)(C)OC(=O)CCN(C(=O)CCN1C(=O)C=CC1=O)N(CCC(=O)OC(C)(C)C)C(=O)CCN1C(=O)C=CC1=O]

3-Maleido-propanoic acid (1.00 g, 5.91 mmol) in DCM (50 ml) was added oxalyl dichloride (2.70 g, 21.25 mmol) and DMF (50 μL). The mixture was stirred at room temperature for 2 h, evaporated, and co-evaporated with DCM/toluene to obtain crude 3-maleido-propanoic acid chloride. To the compound di-tert-Butyl 3,3′-(hydrazine-1,2-diyl)dipropanoate (0.51 g, 1.76 mmol) in the mixture of DCM (35 ml) was added the crude 3-maleido-propanoic acid chloride. The mixture was stirred for overnight, evaporated, concentrated and purified on SiO₂ column eluted with EtOAc/DCM (1:15˜1:8) to afford the title compound (738 mg, 71% yield). ESI MS m/z+ C₂₈H₃₈N₄NaO₁₀ (M+Na), cacld. 613.26. found 613.40.

### Example 14. Synthesis of 3,3′-(1,2-bis(3-(2,5-dioxo-2,5-dihydro-1H-pyrrol-1-yl)propanoyl)-hydrazine-1,2-diyl)dipropanoic Acid

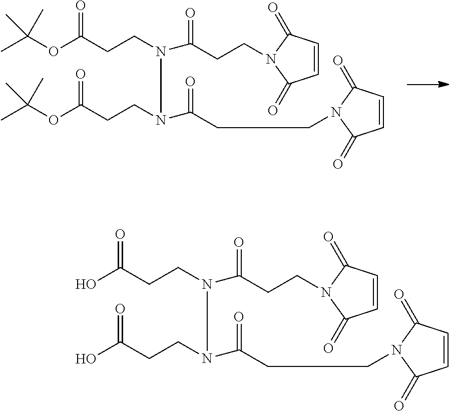 [SMILES: CC(C)(C)OC(=O)CCN(C(=O)CCN1C(=O)C=CC1=O)N(CCC(=O)OC(C)(C)C)C(=O)CCN1C(=O)C=CC1=O ; O=C(O)CCN(C(=O)CCN1C(=O)C=CC1=O)N(CCC(=O)O)C(=O)CCN1C(=O)C=CC1=O]

Compound 14 (700 mg, 1.18 mmol) in dioxane (4 ml) was added HCl (conc. 1 ml). The mixture was stirred for 30 min, diluted with EtOH (10 mL) and toluene (10 ml), evaporated and coevaporated with EtOH (10 ml) and toluene (10 ml) to afford the crude title product (560 mg) for next step without further purification. ESI MS m/z− C₂₀H₂₁N₄O₁₀ (M−H), cacld. 477.13. found 477.20.

### Example 15. Synthesis of Bis(2,5-dioxopyrrolidin-1-yl)-3,3′-(1,2-bis(3-(2,5-dioxo-2,5-dihydro-1H-pyrrol-1-yl)propanoyl)hydrazine-1,2-diyl)dipropanoate

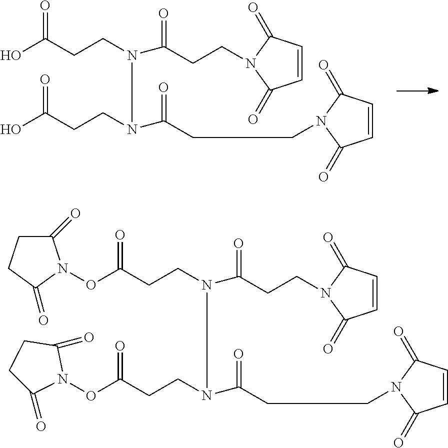 [SMILES: O=C(O)CCN(C(=O)CCN1C(=O)C=CC1=O)N(CCC(=O)O)C(=O)CCN1C(=O)C=CC1=O ; O=C(CCN(C(=O)CCN1C(=O)C=CC1=O)N(CCC(=O)ON1C(=O)CCC1=O)C(=O)CCN1C(=O)C=CC1=O)ON1C(=O)CCC1=O]

To the crude compound 3,3′-(1,2-bis(3-(2,5-dioxo-2,5-dihydro-1H-pyrrol-1-yl)propanoyl)-hydrazine-1,2-diyl)dipropanoic acid (˜560 mg, 1.17 mmol) in DMA (8 ml) was added NHS (400 mg, 3.47 mmol) and EDC (1.01 g, 5.26 mmol). The mixture was stirred for overnight, evaporated, concentrated and purified on SiO₂ column eluted with EtOAc/DCM (1:12˜1:7) to afford the title compound (520 mg, 65% yield in 2 steps). ESI MS m/z+ C₂₈H₂₈N₆NaO₁₄ (M+Na), cacld. 695.17. found 695.40.

### Example 16. Synthesis of tert-Butyl 3-(2-(2-(2-hydroxyethoxy)ethoxy)ethoxy)propanoate

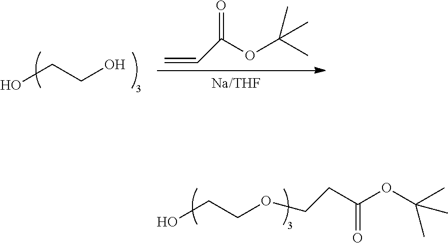

To 350 mL of anhydrous THF was added 80 mg (0.0025 mol) of sodium metal and triethylene glycol 150.1 g, 1.00 mol) with stirring. After the sodium had completely dissolved, tert-butyl acrylate (24 mL, 0.33 mol) was added. The solution was stirred for 20 h at room temperature and neutralized with 8 mL of 1.0 M HCl. The solvent was removed in vacuo and the residue was suspended in brine (250 mL) and extracted with ethyl acetate (3×125 mL). The combined organic layers were washed with brine (100 mL) then water (100 mL), dried over sodium sulfate, and the solvent was removed. The resulting colorless oil was dried under vacuum to give 69.78 g (76% yields) of the title product. ¹H NMR: 1.41 (s, 9H), 2.49 (t, 2H, J=6.4 Hz), 3.59-3.72 (m, 14H). ESI MS m/z− C₁₃H₂₅O₆(M−H), cacld. 277.17. found 277.20.

### Example 17. Synthesis of tert-Butyl 3-(2-(2-(2-(tosyloxy)ethoxy)ethoxy)ethoxy)propanoate

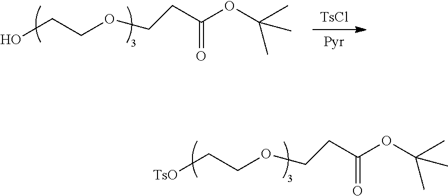

A solution of tert-Butyl 3-(2-(2-(2-hydroxyethoxy)ethoxy)ethoxy)propanoate (10.0 g, 35.95 mmol) in acetonitrile (50.0 mL) was treated with pyridine (20.0 mL). A solution of tosyl chloride (7.12 g, 37.3 mmol) in 50 mL acetonitrile was added dropwise via an addition funnel over 30 minutes. After 5 h TLC analysis revealed that the reaction was complete. The pyridine hydrochloride that had formed was filtered off and the solvent was removed. The residue was purified on silica gel by eluting from with 20% ethyl acetate in hexane to with neat ethyl acetate to give 11.2 g (76% yield) of the title compound. ¹H NMR: 1.40 (s, 9H), 2.40 (s, 3H), 2.45 (t, 2H, J=6.4 Hz), 3.52-3.68 (m, 14H), 4.11 (t, 2H, J=4.8 Hz), 7.30 (d, 2H, J=8.0 Hz), 7.75 (d, 2H, J=8.0 Hz); ESI MS m/z+ C₂₀H₃₃O₈S (M+H), cacld. 433.18. found 433.30.

### Example 18. Synthesis of tert-Butyl 3-(2-(2-(2-azidoethoxy)ethoxy)ethoxy)propanoate

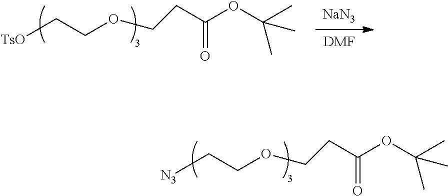

To 50 mL of DMF was added tert-butyl 3-(2-(2-(2-(tosyloxy)ethoxy)ethoxy)ethoxy)propanoate (4.0 g, 9.25 mmol) and sodium azide (0.737 g, 11.3 mmol) with stirring. The reaction was heated to 80° C. After 4 h TLC analysis revealed that the reaction was complete. The reaction was cooled to room temperature and quenched with water (25 mL). The aqueous layer was separated and extracted into ethyl acetate (3×35 mL). The combined organic layers were dried over anhydrous magnesium sulfate, filtered, and the solvent removed in vacuo. The crude azide product (2.24 g, 98% yield, about 93% pure by HPLC) was used for next step without further purification. ¹H NMR (CDCl₃): 1.40 (s, 9H), 2.45 (t, 2H, J=6.4 Hz), 3.33 (t, 2H, J=5.2 Hz), 3.53-3.66 (m, 12H). ESI MS m/z+ C₁₃H₂₆N₃O₈ (M+H), cacld. 304.18. found 304.20.

### Example 19. Synthesis of 3-(2-(2-(2-azidoethoxy)ethoxy)ethoxy)propanoic Acid

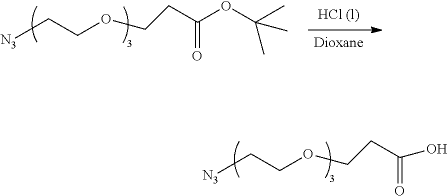

Tert-butyl 3-(2-(2-(2-azidoethoxy)ethoxy)ethoxy)propanoate (2.20 g, 7.25 mmol) in 1,4-dioxane (40 ml) was added HCl (12 M, 10 ml). The mixture was stirred for 40 min, diluted with dioxane (20 ml) and toluene (40 ml), evaporated and co-evaporated with dioxane (20 ml) and toluene (40 ml) to dryness to afford the crude title product for the next step without further production (1.88 g, 105% yield, ˜92% pure by HPLC). MS ESI m/z calcd for C₉H₁₈N₃O₅ [M+H]⁺ 248.12. found 248.40.

### Example 20. Synthesis of 13-Amino-4,7,10-trioxadodecanoic Acid Tert-Butyl Ester, and 13-Amino-bis(4,7,10-trioxadodecanoic acid tert-Butyl Ester)

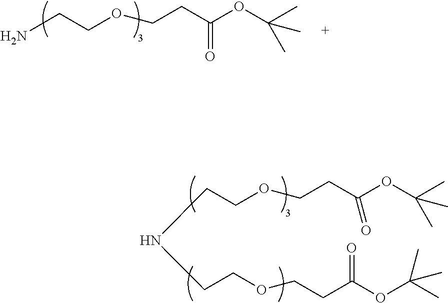

The crude azide material 3-(2-(2-(2-azidoethoxy)ethoxy)ethoxy)propanoic acid (5.0 g, ˜14.84 mmol) was dissolved in ethanol (80 mL) and 300 mg of 10% Pd/C was added. The system was evacuated under vacuum and placed under 2 atm of hydrogen gas via hydrogenation reactor with vigorous stirring. The reaction was then stirred overnight at room temperature and TLC showed that the starting materials disappeared. The crude reaction was passed through a short pad of Celite rinsing with ethanol. The solvent was removed and the amine purified on silica gel using a mixture of methanol (from 5% to 15%) and 1% triethylamine in methylene chloride as the eluant to give 13-amino-4,7,10-trioxadodecanoic acid tert-butyl ester (1.83 g, 44% yield, ESI MS m/z+ C₁₃H₂₇NO₅ (M+H), cacld. 278.19. found 278.30) and 13-amino-bis(4,7,10-trioxadodecanoic acid tert-butyl ester) (2.58 g, 32% yield, ESI MS m/z+ C₂₆H₅₂NO₁₀ (M+H), cacld. 538.35. found 538.40).

### Example 21. Synthesis of 3-(2-(2-(2-Aminoethoxy)ethoxy)ethoxy)propanoic Acid, HCl Salt

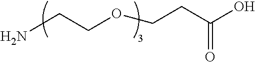

To 13-amino-4,7,10-trioxadodecanoic acid tert-butyl ester (0.80 g, 2.89 mmol) in 30 mL of dioxane was 10 ml of HCl (36%) with stirring. After 0.5 h TLC analysis revealed that the reaction was complete, the reaction mixture was evaporated, and co-evaporated with EtOH and EtOH/Toluene to form the title product in HCl salt (>90% pure, 0.640 g, 86% yield) without further purification. ESI MS m/z+ C₉H₂₀NO₅ (M+H), cacld. 222.12. found 222.20.

### Example 22. 13-Amino-bis(4,7,10-trioxadodecanoic Acid, HCl Salt

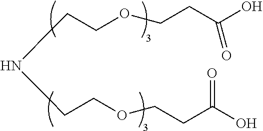

To 13-amino-bis(4,7,10-trioxadodecanoic acid tert-butyl ester) (1.00 g, 1.85 mmol) in 30 mL of dioxane was 10 ml of HCl (36%) with stirring. After 0.5 h TLC analysis revealed that the reaction was complete, the reaction mixture was evaporated, and co-evaporated with EtOH and EtOH/Toluene to form the title product in HCl salt (>90% pure, 0.71 g, 91% yield) without further purification. ESI MS m/z+ C₁₈H₃₆NO₁₀ (M+H), cacld. 426.22. found 426.20.

### Example 23. Synthesis of tert-butyl 3-(2-(2-(2-hydroxyethoxy)ethoxy)ethoxy) propanoate

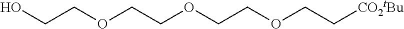 [SMILES: CC(C)(C)OC(=O)CCOCCOCCOCCO]

To a solution of 2,2′-(ethane-1,2-diylbis(oxy))diethanol (55.0 mL, 410.75 mmol, 3.0 eq.) in anhydrous THF (200 mL) was added sodium (0.1 g). The mixture was stirred until Na disappeared and then tert-butyl acrylate (20.0 mL, 137.79 mmol, 1.0 eq.) was added dropwise. The mixture was stirred overnight and then quenched by HCl solution (20.0 mL, 1N) at 0° C. THF was removed by rotary evaporation, brine (300 mL) was added and the resulting mixture was extracted with EtOAc (3×100 mL). The organic layers were washed with brine (3×300 mL), dried over anhydrous Na₂SO₄, filtered and concentrated to afford a colourless oil (30.20 g, 79.0% yield), which was used without further purification. MS ESI m/z calcd for C₁₃H₂₇O₆ [M+H]⁺278.1729. found 278.1730.

### Example 24. Synthesis of tert-butyl 3-(2-(2-(2-(tosyloxy)ethoxy)ethoxy)ethoxy) propanoate

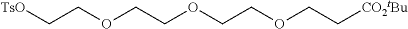 [SMILES: Cc1ccc(S(=O)(=O)OCCOCCOCCOCCC(=O)OC(C)(C)C)cc1]

To a solution of tert-butyl 3-(2-(2-(2-hydroxyethoxy)ethoxy)ethoxy) propanoate (30.20 g, 108.5 mmol, 1.0 eq.) and TsCl (41.37 g, 217.0 mmol, 2.0 eq.) in anhydrous DCM (220 mL) at 0° C. was added TEA (30.0 mL, 217.0 mmol, 2.0 eq.). The mixture was stirred at room temperature overnight, and then washed with water (3×300 mL) and brine (300 mL), dried over anhydrous Na₂SO₄, filtered, concentrated and purified by SiO₂ column chromatography (3:1 hexanes/EtOAc) to give a colourless oil (39.4 g, 84.0% yield). MS ESI m/z calcd for C₂₀H₃₃O₈S [M+H]⁺ 433.1818. found 433.2838.

### Example 25. Synthesis of tert-butyl 3-(2-(2-(2-azidoethoxy)ethoxy)ethoxy) propanoate

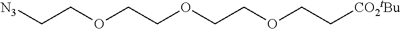 [SMILES: CC(C)(C)OC(=O)CCOCCOCCOCCN=[N+]=[N-]]

To a solution of tert-butyl 3-(2-(2-(2-(tosyloxy)ethoxy)ethoxy)ethoxy) propanoate (39.4 g, 91.1 mmol, 1.0 eq.) in anhydrous DMF (100 mL) was added NaN₃ (20.67 g, 316.6 mmol, 3.5 eq.). The mixture was stirred at room temperature overnight. Water (500 mL) was added and extracted with EtOAc (3×300 mL). The combined organic layers were washed with water (3×900 mL) and brine (900 mL), dried over anhydrous Na₂SO₄, filtered, concentrated and purified by SiO₂ column chromatography (5:1 hexanes/EtOAc) to give a light yellow oil (23.8 g, 85.53% yield). MS ESI m/z calcd for C₁₃H₂₅O₃N₅Na [M+Na]⁺326.2. found 326.2.

### Example 26. Synthesis of tert-butyl 3-(2-(2-(2-aminoethoxy)ethoxy)ethoxy) propanoate

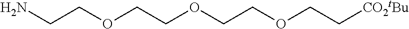 [SMILES: CC(C)(C)OC(=O)CCOCCOCCOCCN]

Raney-Ni (7.5 g, suspended in water) was washed with water (three times) and isopropyl alcohol (three times) and mixed with tert-butyl 3-(2-(2-(2-azidoethoxy)ethoxy)ethoxy) propanoate (5.0 g, 16.5 mmol) in isopropyl alcohol. The mixture was stirred under a H₂ balloon at r.t. for 16 h and then filtered over a Celite pad, with washing of the pad with isopropyl alcohol. The filtrate was concentrated and purified by column chromatography (5-25% MeOH/DCM) to give a light yellow oil (2.60 g, 57% yield). MS ESI m/z calcd for C₁₃H₂₈NO₅ [M+H]⁺ 279.19; found 279.19.

### Example 27. Synthesis of 2-(2-(dibenzylamino)ethoxy)ethanol

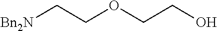

2-(2-aminoethoxy)ethanol (21.00 g, 200 mmol, 1.0 eq.) and K₂CO₃ (83.00 g, 600 mmol, 3.0 eq.) in acetonitrile (350 mL) was added BnBr (57.0 mL, 480 mmol, 2.4 eq.). The mixture was refluxed overnight. Water (1 L) was added and extracted with EtOAc (3×300 mL). The combined organic layers were washed with brine (1000 mL), dried over anhydrous Na₂SO₄, filtered, concentrated and purified by SiO₂ column chromatography (4:1 hexanes/EtOAc) to give a colourless oil (50.97 g, 89.2% yield). MS ESI m/z calcd for C₁₈H₂₃NO₂Na [M+Na]⁺ 309.1729. found 309.1967.

### Example 28. Synthesis of tert-butyl 3-(2-(2-(dibenzylamino)ethoxy)ethoxy) propanoate

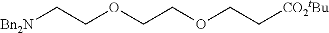

To a mixture of 2-(2-(dibenzylamino)ethoxy)ethanol (47.17 g, 165.3 mmol, 1.0 eq.), tert-butyl acrylate (72.0 mL, 495.9 mmol, 3.0 eq.) and n-Bu₄NI (6.10 g, 16.53 mmol, 0.1 eq.) in DCM (560 mL) was added sodium hydroxide solution (300 mL, 50%). The mixture was stirred overnight. The organic layer was separated and the water layer was extracted with EtOAc (3×100 mL). The organic layers were washed with water (3×300 mL) and brine (300 mL), dried over anhydrous Na₂SO₄, filtered, concentrated and purified by SiO₂ column chromatography (7:1 hexanes/EtOAc) to give a colourless oil (61.08 g, 89.4% yield). MS ESI m/z calcd for C₂₅H₃₆NO₄ [M+H]⁺ 414.2566. found 414.2384.

### Example 29. Synthesis of tert-butyl 3-(2-(2-aminoethoxy)ethoxy)propanoate

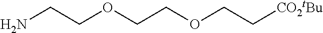 [SMILES: CC(C)(C)OC(=O)CCOCCOCCN]

To a solution of tert-butyl 3-(2-(2-(dibenzylamino)ethoxy)ethoxy) propanoate (20.00 g, 48.36 mmol, 1.0 eq.) in THF (30 mL) and MeOH (60 mL) was added Pd/C (2.00 g, 10 wt %, 50% wet) in a hydrogenation bottle. The mixture was shaken at 1 atom pressure H₂ overnight, filtered through Celite (filter aid), and the filtrate was concentrated to afford a colourless oil (10.58 g, 93.8% yield). MS ESI m/z calcd for C₁₁H₂₄NO₄ [M+H]⁺ 234.1627. found 234.1810.

### Example 30. Synthesis of tert-butyl 3-(2-(2-hydroxyethoxy)ethoxy)propanoate

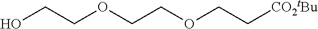 [SMILES: CC(C)(C)OC(=O)CCOCCOCCO]

To a solution of 2,2′-oxydiethanol (19.7 mL, 206.7 mmol, 3.0 eq.) in anhydrous THF (100 mL) was added sodium (0.1 g). The mixture was stirred until Na disappeared and then tert-butyl acrylate (10.0 mL, 68.9 mmol, 1.0 eq.) was added dropwise. The mixture was stirred overnight, and brine (200 mL) was added and extracted with EtOAc (3×100 mL). The organic layers were washed with brine (3×300 mL), dried over anhydrous Na₂SO₄, filtered, concentrated and purified by SiO₂ column chromatography (1:1 hexanes/EtOAc) to give to a colourless oil (8.10 g, 49.4% yield). MS ESI m/z calcd for C₁₁H₂₃O₅[M+H]⁺ 235.1467. found 235.1667.

### Example 31. Synthesis of tert-butyl 3-(2-(2-(tosyloxy)ethoxy)ethoxy)propanoate

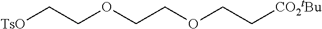 [SMILES: Cc1ccc(S(=O)(=O)OCCOCCOCCC(=O)OC(C)(C)C)cc1]

To a solution of tert-butyl 3-(2-(2-hydroxyethoxy)ethoxy)propanoate (6.24 g, 26.63 mmol, 1.0 eq.) and TsCl (10.15 g, 53.27 mmol, 2.0 eq.) in anhydrous DCM (50 mL) at 0° C. was added pyridine (4.3 mL, 53.27 mmol, 2.0 eq.). The mixture was stirred at room temperature overnight, and then washed with water (100 mL) and the water layer was extracted with DCM (3×50 mL). The combined organic layers were washed with brine (300 mL), dried over anhydrous Na₂SO₄, filtered, concentrated and purified by SiO₂ column chromatography (5:1 hexanes/EtOAc) to give a colourless oil (6.33 g, 61.3% yield). MS ESI m/z calcd for C₁₈H₂₇O₇S [M+H]⁺ 389.1556. found 389.2809.

### Example 32. Synthesis of tert-butyl 3-(2-(2-azidoethoxy)ethoxy)propanoate

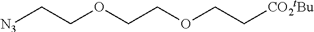 [SMILES: CC(C)(C)OC(=O)CCOCCOCCN=[N+]=[N-]]

To a solution of tert-butyl 3-(2-(2-(tosyloxy)ethoxy)ethoxy)propanoate (5.80 g, 14.93 mmol, 1.0 eq.) in anhydrous DMF (20 mL) was added NaN₃ (5.02 g, 77.22 mmol, 5.0 eq.). The mixture was stirred at room temperature overnight. Water (120 mL) was added and extracted with EtOAc (3×50 mL). The combined organic layers were washed with water (3×150 mL) and brine (150 mL), dried over anhydrous Na₂SO₄, filtered, concentrated and purified by SiO₂ column chromatography (5:1 hexanes/EtOAc) to give a colourless oil (3.73 g, 69.6% yield). MS ESI m/z calcd for C₁₁H₂₂O₃N₄Na[M+H]⁺ 260.1532. found 260.2259.

### Example 33. Synthesis of tert-butyl 3-(2-(2-aminoethoxy)ethoxy)propanoate

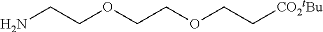 [SMILES: CC(C)(C)OC(=O)CCOCCOCCN]

tert-Butyl 3-(2-(2-azidoethoxy)ethoxy)propanoate (0.18 g, 0.69 mmol) was dissolved in MeOH (3.0 mL, with 60 μL concentrated HCl) and hydrogenated with Pd/C (10 wt %, 20 mg) under a H₂ balloon for 30 min. The catalyst was filtered through a Celite pad, with washing of the pad with MeOH. The filtrate was concentrated to give a colorless oil (0.15 g, 93% yield). MS ESI m/z calcd for C₁₁H₂₄NO₄ [M+H]⁺ 234.16; found 234.14.

### Example 34. Synthesis of 3-(2-(2-azidoethoxy)ethoxy)propanoic Acid

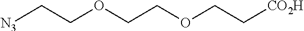 [SMILES: [N-]=[N+]=NCCOCCOCCC(=O)O]

tert-Butyl 3-(2-(2-azidoethoxy)ethoxy)propanoate (2.51 g, 9.68 mmol) dissolved in 1,4-dioxane (30 mL) was treated with 10 ml of HCl (conc.) at r.t. The mixture was stirred for 35 min, diluted with EtOH (30 ml) and toluene (30 ml) and concentrated under vacuum. The crude mixture was purified on silica gel using a mixture of methanol (from 5% to 10%) and 1% formic acid in methylene chloride as the eluant to give title compound (1.63 g, 83% yield), ESI MS m/z C₇H₁₂N₃O₄ [M−H]⁻, cacld. 202.06. found 202.30.

### Example 35. Synthesis of 2,5-dioxopyrrolidin-1-yl 3-(2-(2-azidoethoxy)ethoxy)propanoate

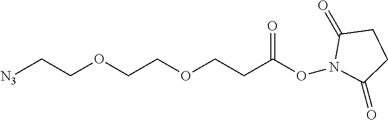 [SMILES: [N-]=[N+]=NCCOCCOCCC(=O)ON1C(=O)CCC1=O]

To 3-(2-(2-azidoethoxy)ethoxy)propanoic acid (1.60 g, 7.87 mmol) in 30 mL of dichloromethane was added NHS (1.08 g, 9.39 mmol) and EDC (3.60 g, 18.75 mmol) with stirring. After 8 h TLC analysis revealed that the reaction was complete, the reaction mixture was concentrated and purified on silica gel using a mixture of ethyl acetate (from 5% to 10%) in methylene chloride as the eluant to give title compound (1.93 g, 82% yield). ESI MS m/z C₁₁H₁₇N₄O₆[M+H]⁺, cacld. 301.11. found 301.20.

### Example 36. Synthesis of 2,5-dioxopyrrolidin-1-yl 3-(2-(2-(2-azidoethoxy)ethoxy)ethoxy)propanoate

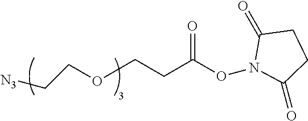

To 3-(2-(2-(2-azidoethoxy)ethoxy)ethoxy)propanoic acid (4.50 g, 18.21 mmol) in 80 mL of dichloromethane was added NHS (3.0 g, 26.08 mmol) and EDC (7.60 g, 39.58 mmol) with stirring. After 8 h TLC analysis revealed that the reaction was complete, the reaction mixture was concentrated and purified on silica gel using a mixture of ethyl acetate (from 5% to 10%) in methylene chloride as the eluant to give title compound (5.38 g, 86% yield). ESI MS m/z C₁₃H₂₀N₄O₇ [M+H]⁺, cacld. 345.13. found 345.30.

### Example 37. Synthesis of (14S,17S)-1-azido-17-(2-(tert-butoxy)-2-oxoethyl)-14-(4-((tert-butoxycarbonyl)-amino)butyl)-12,15-dioxo-3,6,9-trioxa-13,16-diazaoctadecan-18-oic Acid

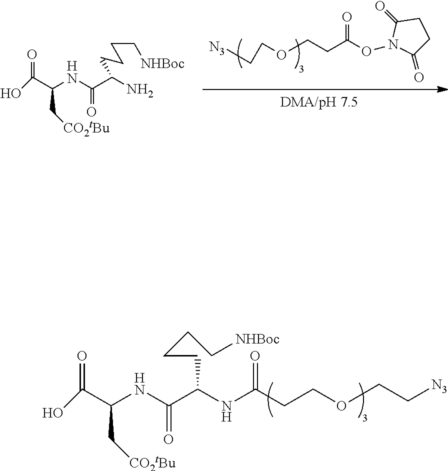

To a solution of (S)-2-((S)-2-amino-6-((tert-butoxycarbonyl)amino)hexanamido)-4-(tert-butoxy)-4-oxobutanoic acid (2.81 g, 6.73 mmol) in the mixture of DMA (70 ml) and 0.1 M NaH₂PO₄ (50 ml, pH 7.5) was added 2,5-dioxopyrrolidin-1-yl 3-(2-(2-(2-azidoethoxy)ethoxy)-ethoxy)propanoate (3.50 g, 10.17). The mixture was stirred for 4 h, evaporated in vacuo, purified on silica gel using a mixture of methanol (from 5% to 15%) in methylene chloride containing 0.5% acetic acid as the eluant to give title compound (3.35 g, 77% yield). ESI MS m/z C₂₈H₅₁N₆O₁₁ [M+H]⁺, cacld. 647.35. found 647.80.

### Example 38. Synthesis of (14S,17S)-tert-butyl 1-azido-14-(4-((tert-butoxycarbonyl)amino)butyl)-17-((4-(hydroxymethyl)phenyl)carbamoyl)-12,15-dioxo-3,6,9-trioxa-13,16-diazanonadecan-19-oate

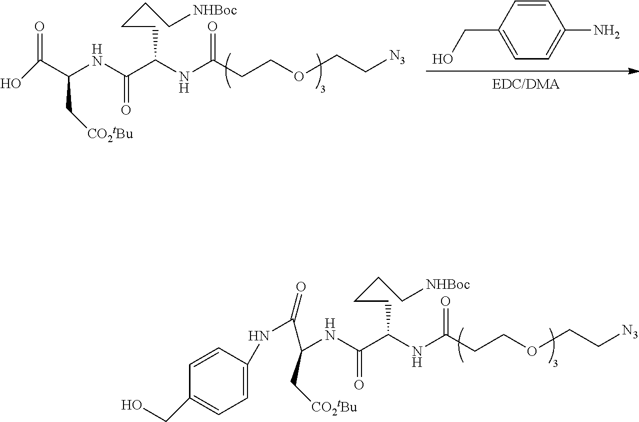

(14S,17S)-1-azido-17-(2-(tert-butoxy)-2-oxoethyl)-14-(4-((tert-butoxycarbonyl)-amino)butyl)-12,15-dioxo-3,6,9-trioxa-13,16-diazaoctadecan-18-oic acid (3.30 g, 5.10 mmol) and (4-aminophenyl)methanol (0.75 g, 6.09) in DMA (25 ml) was added EDC (2.30 g, 11.97 mmol). The mixture was stirred for overnight, evaporated in vacuo, purified on silica gel using a mixture of methanol (from 5% to 8%) in methylene chloride containing as the eluant to give title compound (3.18 g, 83% yield). ESI MS m/z C₃₅H₅₈N₇O₁₁ [M+H]⁺, cacld. 752.41. found 752.85.

### Example 39. Synthesis of (14S,17S)-tert-butyl 1-amino-14-(4-((tert-butoxycarbonyl)amino)butyl)-17-((4-(hydroxymethyl)phenyl)carbamoyl)-12,15-dioxo-3,6,9-trioxa-13,16-diazanonadecan-19-oate

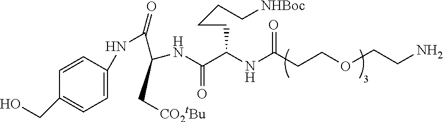

To a solution of (14S,17S)-tert-butyl 1-azido-14-(4-((tert-butoxycarbonyl)amino)butyl)-17-((4-(hydroxymethyl)phenyl)carbamoyl)-12,15-dioxo-3,6,9-trioxa-13,16-diazanonadecan-19-oate (1.50 g, 1.99 mmol) in THF (35 mL) was added Pd/C (200 mg, 10% Pd, 50% wet) in a hydrogenation bottle. The mixture was shaken at 1 atom pressure H₂ overnight, filtered through Celite (filter aid), and the filtrate was concentrated to afford the title compound (1.43 g, 99% yield) which was used immediately for the next step without further purification. ESI MS m/z C₃₅H₆₀N₅O₁₁ [M+H]⁺, cacld. 726.42. found 726.70.

### Example 40. Synthesis of (S)-15-azido-5-isopropyl-4,7-dioxo-10,13-dioxa-3,6-diazapentadecan-1-oic Acid

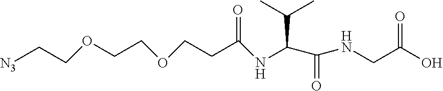 [SMILES: CC(C)[C@H](NC(=O)CCOCCOCCN=[N+]=[N-])C(=O)NCC(=O)O]

To a solution of (S)-2-(2-amino-3-methylbutanamido)acetic acid (Val-Gly) (1.01 g, 5.80 mmol) in the mixture of DMA (50 ml) and 0.1 M NaH₂PO₄ (50 ml, pH 7.5) was added 2,5-dioxopyrrolidin-1-yl 3-(2-(2-azidoethoxy)ethoxy)propanoate (1.90 g, 6.33). The mixture was stirred for 4 h, evaporated in vacuo, purified on silica gel using a mixture of methanol (from 5% to 15%) in methylene chloride containing 0.5% acetic acid as the eluant to give title compound (1.52 g, 73% yield). ESI MS m/z C₁₄H₂₆N₅O₆ [M+H]⁺, cacld. 360.18. found 360.40.

### Example 41. Synthesis of (S)-2,5-dioxopyrrolidin-1-yl 15-azido-5-isopropyl-4,7-dioxo-10,13-dioxa-3,6-diazapentadecan-1-oate

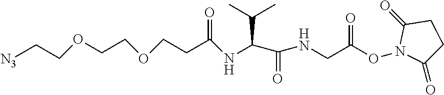 [SMILES: CC(C)[C@H](NC(=O)CCOCCOCCN=[N+]=[N-])C(=O)NCC(=O)ON1C(=O)CCC1=O]

To a solution of (S)-15-azido-5-isopropyl-4,7-dioxo-10,13-dioxa-3,6-diazapentadecan-1-oic acid (1.50 g, 4.17 mmol) in 40 mL of dichloromethane was added NHS (0.88 g, 7.65 mmol) and EDC (2.60 g, 13.54 mmol) with stirring. After 8 h TLC analysis revealed that the reaction was complete, the reaction mixture was concentrated and purified on silica gel using a mixture of ethyl acetate (from 5% to 20%) in methylene chloride as the eluant to give title compound (1.48 g, 78% yield). ESI MS m/z C₁₈H₂₉N₆O₈ [M+H]⁺, cacld. 457.20. found 457.50.

### Example 42. Synthesis of 4-(((benzyloxy)carbonyl)amino)butanoic Acid

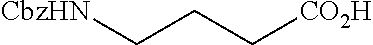 [SMILES: O=C(O)CCCNC(=O)OCc1ccccc1]

A solution of 4-aminobutyric acid (7.5 g, 75 mmol) and NaOH (6 g, 150 mmol) in H₂O (40 mL) was cooled to 0° C. and treated with a solution of CbzCl (16.1 g, 95 mmol) in THF (32 ml) dropwise. After 1 h, the reaction was allowed to warm to r.t. and stirred for 3 h. THF was removed under vacuum, the pH of the aqueous solution was adjusted to 1.5 by addition of 6 N HCl. Extracted with ethyl acetate, and the organic layer was washed with brine, dried and concentrated to give the title compound (16.4 g, 92% yield). MS ESI m/z calcd for C₁₂H₁₆NO₅ [M+H]⁺ 238.10. found 238.08.

### Example 43. Synthesis of tert-butyl 4-(((benzyloxy)carbonyl)amino)butanoate

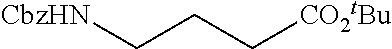 [SMILES: CC(C)(C)OC(=O)CCCNC(=O)OCc1ccccc1]

DMAP (0.8 g, 6.56 mmol) and DCC (17.1 g, 83 mmol) were added to a solution of 4-(((benzyloxy)carbonyl)amino)butanoic acid (16.4 g, 69.2 mmol) and t-BuOH (15.4 g, 208 mmol) in DCM (100 mL). After stirring at r.t. overnight, the reaction was filtered and filtrate concentrated. The residue was dissolved in ethyl acetate and the washed with 1N HCl, brine and dried over Na₂SO₄. Concentration and purification by column chromatography (10 to 50% EtOAc/hexanes) yielded the title compound (7.5 g, 37% yield). MS ESI m/z calcd for C₁₆H₂₃NO₄Na [M+Na]⁺316.16. found 316.13.

### Example 44. Synthesis of tert-butyl 4-aminobutanoate

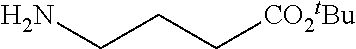 [SMILES: CC(C)(C)OC(=O)CCCN]

tert-Butyl 4-(((benzyloxy)carbonyl)amino)butanoate (560 mg, 1.91 mmol) was dissolved in MeOH (50 mL), and mixed with Pd/C catalyst (10 wt %, 100 mg) then hydrogenated (1 atm) at room temperature for 3 h. The catalyst was filtered off and all volatiles were removed under vacuum to afford the title compound (272 mg, 90% yield). MS ESI m/z calcd for C₈H₁₈NO₂ [M+H]⁺ 160.13. found 160.13.

### Example 45. Synthesis of di-tert-butyl 3,3′-(benzylazanediyl)dipropanoate

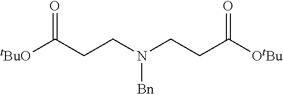 [SMILES: CC(C)(C)OC(=O)CCN(CCC(=O)OC(C)(C)C)Cc1ccccc1]

A mixture of phenylmethanamine (2.0 mL, 18.29 mmol, 1.0 eq) and tert-butyl acrylate (13.3 mL, 91.46 mmol, 5.0 eq) was refluxed at 80° C. overnight and then concentrated. The crude product was purified by SiO₂ column chromatography (20:1 hexanes/EtOAc) to give the title compound as colourless oil (5.10 g, 77% yield). ESI MS m/z: calcd for C₂₁H3₄NO₄ [M+H]⁺ 364.2. found 364.2. ¹H NMR (400 MHz, CDCl₃) δ 7.38-7.21 (m, 5H), 3.58 (s, 2H), 2.76 (t, J=7.0 Hz, 4H), 2.38 (t, J=7.0 Hz, 4H), 1.43 (s, 17H).

### Example 46. Synthesis of di-tert-butyl 3,3′-azanediyldipropanoate

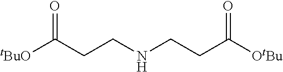 [SMILES: CC(C)(C)OC(=O)CCNCCC(=O)OC(C)(C)C]

To a solution of di-tert-butyl 3,3′-(benzylazanediyl)dipropanoate (1.37 g, 3.77 mmol, 1.0 equiv) in MeOH (10 mL) was added Pd/C (0.20 g, 10% Pd/C, 50% wet) in a hydrogenation bottle. The mixture was shaken overnight under H₂ atmosphere and then filtered through a Celite pad. The filtrate was concentrated to give the title compound as colourless oil (1.22 g, 89% yield). ESI MS m/z: calcd for C₁₄H₂₈NO₄ [M+H]⁺ 274.19. found 274.20.

### Example 47. Synthesis of tert-butyl 4-(2-(((benzyloxy)carbonyl)amino)propanamido)-butanoate

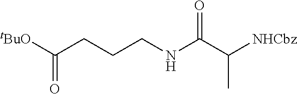 [SMILES: CC(NC(=O)OCc1ccccc1)C(=O)NCCCC(=O)OC(C)(C)C]

To a solution of tert-butyl 4-aminobutanoate (1.00 g, 6.28 mmol, 1.0 eq.) and Z-L-alaine (2.10 g, 9.42 mmol, 1.5 eq.) in anhydrous DCM (50 mL) at 0° C. were added HATU (3.10 g, 8.164 mmol, 1.3 eq.) and TEA (2.6 mL, 18.8 mmol, 3.0 eq.). The reaction was stirred at 0° C. for 10 min., then warmed to room temperature and stirred overnight. The mixture was diluted with DCM and washed with water and brine, dried over anhydrous Na₂SO₄, concentrated and purified by SiO₂ column chromatography (10:3 petroleum ether/ethyl acetate) to give the title compound as a colorless oil (1.39 g, 61% yield). ESI MS m/z: calcd for C₁₉H₂₉N₂O₅Na [M+H]⁺ 387.2. found 387.2.

### Example 48. Synthesis of tert-butyl 4-(2-aminopropanamido)butanoate

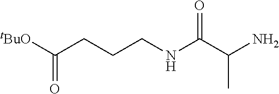 [SMILES: CC(N)C(=O)NCCCC(=O)OC(C)(C)C]

To a solution of tert-butyl 4-(2-(((benzyloxy)carbonyl)amino)propanamido) butanoate (1.39 g, 3.808 mmol, 1.0 eq.) in MeOH (12 mL) was added Pd/C (0.20 g, 10 wt %, 10% wet) in a hydrogenation bottle. The mixture was shaken for 2 h and then filtered through Celite (filter aid), concentrated to give the title compound as a light yellow oil (0.838 g, 95% yield). ESI MS m/z: calcd. for C₁₁H₂₃N₂O₃ [M+H]⁺ 231.16. found 231.15.

### Example 49. Synthesis of 3-(2-(2-(dibenzylamino)ethoxy)ethoxy)propanoic Acid

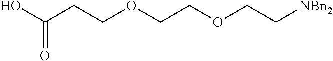

To a solution of tert-butyl 3-(2-(2-(dibenzylamino)ethoxy)ethoxy)propanoate (2.3 g, 5.59 mmol, 1.0eq) in DCM (10 mL) at room temperature was added TFA (5 mL). After stirring for 90 min., the reaction mixture was diluted with anhydrous toluene and concentrated, this operation was repeated for three times to give the title compound as a light yellow oil (2.0 g, theoretical yield), which was directly used in the next step. ESI MS m/z calcd. for C₂₁H₂₈NO₄ [M+H]⁺ 358.19. found 358.19.

### Example 50. Synthesis of perfluorophenyl 3-(2-(2-(dibenzylamino)ethoxy) ethoxy)-propanoate

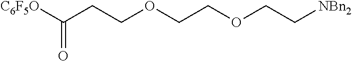

To a solution of 3-(2-(2-(dibenzylamino)ethoxy)ethoxy)propanoic acid (2.00 g, 5.59 mmol, 1.0 eq.) in anhydrous DCM (30 mL) at 0° C. was added DIPEA until pH was neutral, and then PFP (1.54 g, 8.38 mmol, 1.5 eq.) and DIC (1.04 mL, 6.70 mmol, 1.2 eq.) were added. After 10 min. the reaction was warmed to room temperature and stirred overnight. The mixture was filtered, concentrated and purified by SiO₂ column chromatography (15:1 petroleum ether/ethyl acetate) to give the title compound as colourless oil (2.10 g, 72% yield). ESI MS m/z: calcd. for C₂₇H₂₇F₅NO₄ [M+H]⁺ 524.2. found 524.2.

### Example 51. Synthesis of tert-butyl 2-benzyl-13-methyl-11,14-dioxo-1-phenyl-5,8-dioxa-2,12,15-triazanonadecan-19-oate

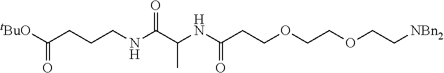

To a solution of tert-butyl 4-(2-aminopropanamido)butanoate (0.736 g, 3.2 mmol, 1.0 eq.) and perfluorophenyl 3-(2-(2-(dibenzylamino)ethoxy) ethoxy)propanoate (2.01 g, 3.84 mmol, 1.2 eq.) in anhydrous DMA (20 mL) at 0° C. was added DIPEA (1.7 mL, 9.6 mmol, 3.0 eq.). After stirring at 0° C. for 10 min. the reaction was warmed to room temperature and stirred overnight. Water (100 mL) was added and the mixture was extracted with EtOAc (3×100 mL). The combined organic layers were washed with water (3×200 mL) and brine (200 mL), dried over Na₂SO₄, filtered, concentrated and purified by SiO₂ column chromatography (25:2 DCM/MeOH) to give the title compound as a colourless oil (1.46 g, 80% yield). ESI MS m/z: calcd. for C₃₂H₄₈N₃O₆ [M+H]⁺ 570.34. found 570.33.

### Example 52. Synthesis of 2-benzyl-13-methyl-11,14-dioxo-1-phenyl-5,8-dioxa-2,12,15-triazanonadecan-19-oic Acid

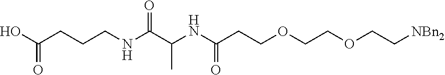

To a solution of tert-butyl 2-benzyl-13-methyl-11,14-dioxo-1-phenyl-5,8-dioxa-2,12,15-triazanonadecan-19-oate (0.057 g, 0.101 mmol, 1.0 eq) in DCM (3 mL) at room temperature was added TFA (1 mL) and stirred for 40 min. The reaction was diluted with anhydrous toluene and then concentrated. This operation was repeated three times to give the title compound as a colourless oil (0.052 g, theoretical yield), which was used directly in the next step. ESI MS m/z: calcd for C₂₈H₄₀N₃O₆ [M+H]⁺ 514.28. found 514.28.

### Example 53. Synthesis of 4-(((benzyloxy)carbonyl)amino)butanoic Acid

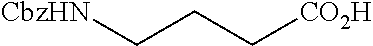 [SMILES: O=C(O)CCCNC(=O)OCc1ccccc1]

A solution of 4-aminobutyric acid (7.5 g, 75 mmol) and NaOH (6 g, 150 mmol) in H2O (40 mL) was cooled to 0° C. and treated with a solution of CbzCl (16.1 g, 95 mmol) in THF (32 ml) dropwise. After 1 h, the reaction was allowed to warm to r.t. and stirred for 3 h. THF was removed under vacuum, the pH of the aqueous solution was adjusted to 1.5 by addition of 6 N HCl. Extracted with ethyl acetate, and the organic layer was washed with brine, dried and concentrated to give the title compound (16.4 g, 92% yield). MS ESI m/z calcd for C12H16NO5 [M+H]+ 238.10. found 238.08.

### Example 54. Synthesis of tert-butyl 4-(((benzyloxy)carbonyl)amino)butanoate

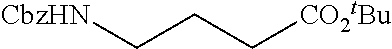 [SMILES: CC(C)(C)OC(=O)CCCNC(=O)OCc1ccccc1]

DMAP (0.8 g, 6.56 mmol) and DCC (17.1 g, 83 mmol) were added to a solution of 4-(((benzyloxy)carbonyl)amino)butanoic acid (16.4 g, 69.2 mmol) and t-BuOH (15.4 g, 208 mmol) in DCM (100 mL). After stirring at r.t. overnight, the reaction was filtered and filtrate concentrated. The residue was dissolved in ethyl acetate and the washed with 1N HCl, brine and dried over Na₂SO₄. Concentration and purification by column chromatography (10 to 50% EtOAc/hexanes) yielded the title compound (7.5 g, 37% yield). MS ESI m/z calcd for C₁₆H₂₃NO₄Na [M+Na]+ 316.16. found 316.13.

### Example 55. Synthesis of tert-butyl 4-aminobutanoate

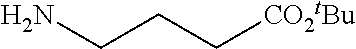 [SMILES: CC(C)(C)OC(=O)CCCN]

tert-Butyl 4-(((benzyloxy)carbonyl)amino)butanoate (560 mg, 1.91 mmol) was dissolved in MeOH (50 mL), and mixed with Pd/C catalyst (10 wt %, 100 mg) then hydrogenated (1 atm) at room temperature for 3 h. The catalyst was filtered off and all volatiles were removed under vacuum to afford the title compound (272 mg, 90% yield). MS ESI m/z calcd for C₈H₁₈NO₂ [M+H]+ 160.13. found 160.13.

### Example 56. Synthesis of tert-butyl 2-(2-(((benzyloxy)carbonyl)amino)propanamido)acetate

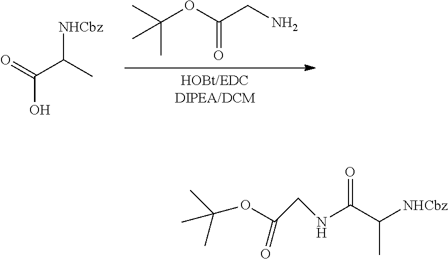

2-(((Benzyloxy)carbonyl)amino)propanoic acid (0.84 g, 5 mmol), tert-butyl 2-aminoacetate (0.66 g, 5 mmol), HOBt (0.68 g, 5 mmol), EDC (1.44 g, 7.5 mmol) were dissolved in DCM (20 ml), followed by addition of DIPEA (1.7 ml, 10 mmol). The reaction mixture was stirred at RT overnight, washed with H₂O (100 ml), and the aqueous layer was extracted with EtOAc. The organic layers were combined, dried over MgSO₄, filtered, evaporated under reduced pressure and the residue was purified on SiO₂ column to give the title product 1 (0.87 g, 52%). ESI: m/z: calcd for C₁₇H2₅N₂O₅ [M+H]⁺: 337.17. found 337.17.

### Example 57. Synthesis of 2-(2-(((benzyloxy)carbonyl)amino)propanamido)acetic Acid

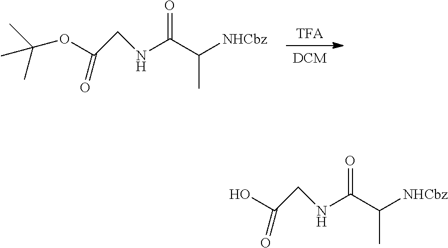

Tert-butyl 2-(2-(((benzyloxy)carbonyl)amino)propanamido)acetate (0.25 g, 0.74 mmol) was dissolved in DCM (30 ml), followed by addition of TFA (10 ml). The mixture was stirred at RT overnight, concentrated to afford the title compound used for the next step without further purification. ESI: m/z: calcd for C₁₃H₁₇N₂O₅ [M+H]⁺: 281.11. found 281.60.

### Example 58. Synthesis of 2,2-dipropiolamidoacetic Acid

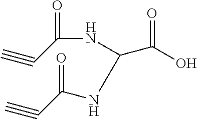

2,2-diaminoacetic acid (2.0 g, 22.2 mmol) in the mixture of EtOH (15 ml) and 50 mM NaH₂PO₄ pH 7.5 buffer (25 ml) was added 2,5-dioxopyrrolidin-1-yl propiolate (9.0 g. 53.8 mmol). The mixture was stirred for 8 h, concentrated, acidified to pH 3.0 with 0.1 M HCl, extracted with EtOAc (3×30 ml). The organic layers were combined, dried over Na₂SO₄, filtered, concentrated and purified on SiO₂ column eluted with MeOH/CH₂Cl₂ (1:10 to 1:6) to afford the title compound (3.27 g, 76% yield). 1H NMR (CDCl₃) 11.8 (br, 1H), 8.12 (d, 2H), 6.66 (m, 1H), 2.66 (s, 2H). ESI MS m/z: calcd for C₈H₆N₂O₄[M+H]⁺ 195.03. found 195.20.

### Example 59. Synthesis of perfluorophenyl 2,2-dipropiolamidoacetate

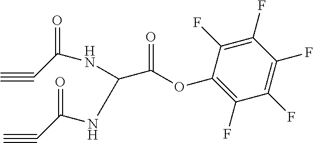 [SMILES: C#CC(=O)NC(NC(=O)C#C)C(=O)Oc1c(F)c(F)c(F)c(F)c1F]

2,2-Dipropiolamidoacetic acid (2.01 g, 10.31 mmol), pentafluorophenol (2.08 g, 11.30 mmol), DIPEA (1.00 ml, 5.73 mmol) and EDC (4.01 g, 20.88 mmol) in CH₂Cl₂ (100 ml) were stirred at RT overnight, concentrated and purified on SiO₂ column eluted with EtOAc/CH₂Cl₂ (1:15 to 1:8) to afford the title compound (3.08 g, 83% yield). 1H NMR (CDCl₃) 8.10 (d, 2H), 6.61 (m, 1H), 2.67 (s, 2H). ESI MS m/z: calcd for C₁₄H6F₅N₂O₄ [M+H]⁺ 361.02. found 361.20.

### Example 60. Synthesis of (S)-2-((S)-2-(2,2-dipropiolamidoacetamido)propanamido)-propanoic Acid

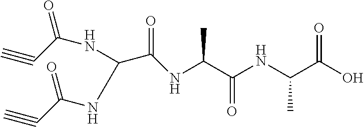 [SMILES: C#CC(=O)NC(NC(=O)C#C)C(=O)N[C@@H](C)C(=O)N[C@@H](C)C(=O)O]

(S)-2-((S)-2-Aminopropanamido)propanoic acid (422) (1.10 g, 6.87 mmol) in the mixture of DMA (18 ml) and 50 mM NaH₂PO₄ pH 7.5 buffer (30 ml) was added perfluorophenyl 2,2-dipropiolamidoacetate (3.00 g. 8.33 mmol). The mixture was stirred for 14 h, concentrated, acidified to pH 3.0 with 0.1 M HCl, extracted with EtOAc (3×40 ml). The organic layers were combined, dried over Na₂SO₄, filtered, concentrated and purified on SiO₂ column eluted with MeOH/CH₂Cl₂ (1:10 to 1:5) to afford the title compound (1.80 g, 78% yield). ESI MS m/z: calcd for C₁₄H₁₇N₄O₆ [M+H]⁺ 337.11. found 337.30.

### Example 61. Synthesis of (S)-2,5-dioxopyrrolidin-1-yl 2-((S)-2-(2,2-dipropiolamido-acetamido)propanamido)propanoate

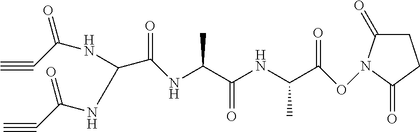 [SMILES: C#CC(=O)NC(NC(=O)C#C)C(=O)N[C@@H](C)C(=O)N[C@@H](C)C(=O)ON1C(=O)CCC1=O]

(S)-2-((S)-2-(2,2-dipropiolamidoacetamido)propanamido)-propanoic acid (1.01 g, 3.00 mmol), NHS (0.41 g, 3.56 mmol), DIPEA (0.40 ml, 2.29 mmol) and EDC (1.51 g, 7.86 mmol) in CH₂Cl₂ (50 ml) were stirred at RT overnight, concentrated and purified on SiO₂ column eluted with EtOAc/CH₂Cl₂ (1:15 to 1:7) to afford the title compound (1.05 g, 81% yield). ESI MS m/z: calcd for C₁₈H₂₀N₅O₈ [M+H]⁺ 434.12. found 434.40.

### Example 62. Synthesis of di-tert-butyl 14,17-dioxo-4,7,10,21,24,27-hexaoxa-13,18-diazatriacont-15-yne-1,30-dioate

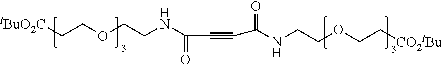

Acetylenedicarboxylic acid (0.35 g, 3.09 mmol, 1.0 eq.) was dissolved in NMP (10 mL) and cooled to 0° C., to which compound tert-butyl 3-(2-(2-(2-aminoethoxy)ethoxy)-ethoxy)propanoate (2.06 g, 7.43 mmol, 2.4 eq.) was added, followed by DMTMM (2.39 g, 8.65 mmol, 2.8 eq.) in portions. The reaction was stirred at 0° C. for 6 h and then diluted with ethyl acetate and washed with water and brine. The organic solution was concentrated and triturated with a mixture solvent of ethyl acetate and petroleum ether. The solid was filtered off and the filtrate was concentrated and purified by column chromatography (80-90% EA/PE) to give a light yellow oil (2.26 g, >100% yield), which was used without further purification. MS ESI m/z [M+H]⁺ 633.30.

### Example 63. Synthesis of 14,17-dioxo-4,7,10,21,24,27-hexaoxa-13,18-diaza triacont-15-yne-1,30-dioic Acid

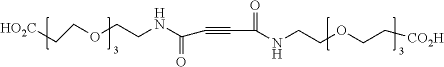

Compound di-tert-butyl 14,17-dioxo-4,7,10,21,24,27-hexaoxa-13,18-diazatriacont-15-yne-1,30-dioate (2.26 g) was dissolved in dichloromethane (15 mL) and cooled to 0° C. then treated with TFA (15 mL). The reaction was warmed to r.t. and stirred for 45 min, and then the solvent and residual TFA was removed on rotovap. The crude product was purified by column chromatography (0-15% MeOH/DCM) to give a light yellow oil (1.39 g, 86% yield for two steps). MS ESI m/z [M+H]⁺ 521.24.

### Example 64. Synthesis of di-tert-butyl 2,5,38,41-tetramethyl-4,7,20,23,36,39-hexaoxo-10,13,16,27,30,33-hexaoxa-3,6,19,24,37,40-hexaazadotetracont-21-yne-1,42-dioate

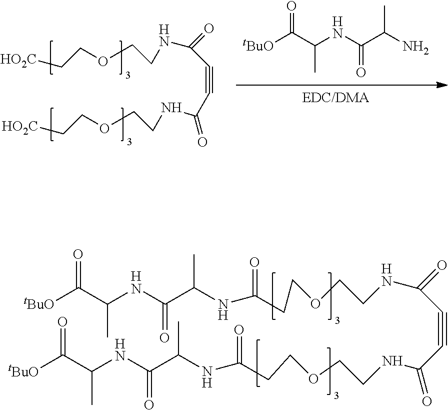

To a solution of 14,17-dioxo-4,7,10,21,24,27-hexaoxa-13,18-diaza triacont-15-yne-1,30-dioic acid (1.38 g, 2.65 mmol), tert-butyl 2-(2-aminopropanamido)propanoate (0.75 g, 3.47 mmol) in the mixture of DMA (40 ml) was added EDC (2.05 g, 10.67 mmol). The mixture was stirred for overnight, concentrated and purified on SiO₂ column eluted with EtOAc/CH₂Cl₂ (1:5 to 1:1) to afford the title compound (2.01 g, 82% yield, ˜95% pure by HPLC). MS ESI m/z calcd for C₄₂H₇₃N₆O₁₆ [M+H]⁺ 917.50. found 917.90.

### Example 65. Synthesis of 2,5,38,41-tetramethyl-4,7,20,23,36,39-hexaoxo-10,13,16,27,30,33-hexaoxa-3,6,19,24,37,40-hexaazadotetracont-21-yne-1,42-dioic Acid

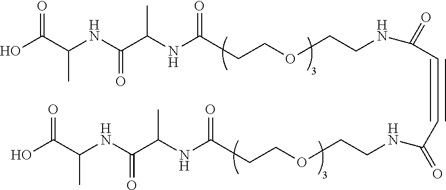

Di-di-tert-butyl 2,5,38,41-tetramethyl-4,7,20,23,36,39-hexaoxo-10,13,16,27,30,33-hexaoxa-3,6,19,24,37,40-hexaazadotetracont-21-yne-1,42-dioate (1.50 g, 1.63 mmol) was dissolved in the mixture of CH₂Cl₂ (10 ml) and TFA (10 ml). The mixture was stirred for overnight, diluted with toluene (20 ml), concentrated to afford the title compound (1.33 g, 101% yield, ˜92% pure by HPLC) which was used for the next step without further purification. MS ESI m/z calcd for C₃₄H₅₆N₆O₁₆ [M+H]⁺ 805.37. found 805.85.

### Example 66. Synthesis of bis(2,5-dioxopyrrolidin-1-yl) 2,5,38,41-tetramethyl-4,7,20,23,36,39-hexaoxo-10,13,16,27,30,33-hexaoxa-3,6,19,24,37,40-hexaazadotetracont-21-yne-1,42-dioate

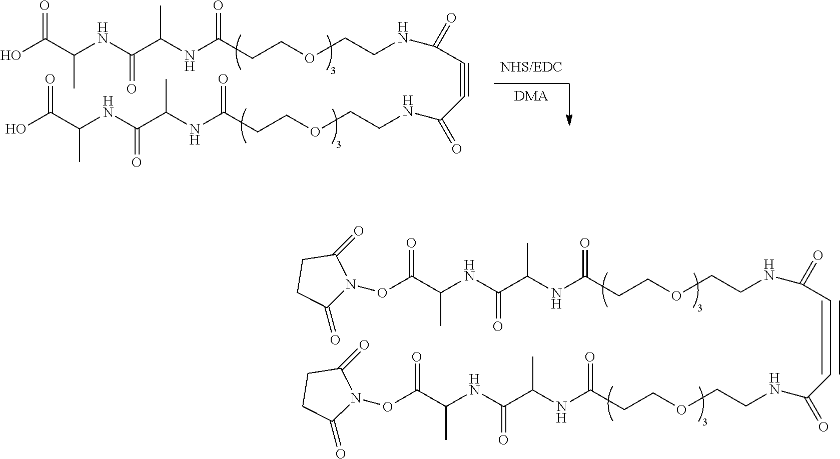

To a solution of 2,5,38,41-tetramethyl-4,7,20,23,36,39-hexaoxo-10,13,16,27,30,33-hexaoxa-3,6,19,24,37,40-hexaazadotetracont-21-yne-1,42-dioic acid (1.30 g, 1.61 mmol) in the mixture of DMA (10 ml) was added NHS (0.60 g, 5.21 mmol) and EDC (1.95 g, 10.15 mmol). The mixture was stirred for overnight, concentrated and purified on SiO₂ column eluted with EtOAc/CH₂Cl₂ (1:4 to 2:1) to afford the title compound (1.33 g, 83% yield, ˜95% pure by HPLC). MS ESI m/z calcd for C₄₂H₆₃N₈O₂₀ [M+H]⁺ 999.40. found 999.95.

### Example 67. Synthesis of 2,3-bis(2-bromoacetamido)succinyl Dichloride

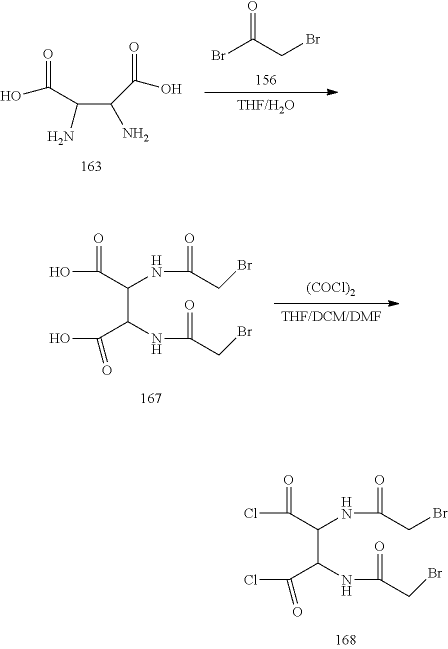

2,3-Diaminosuccinic acid (5.00 g, 33.77 mmol) in the mixture of THF/H₂O/DIPEA (125 ml/125 ml/8 ml) was added 2-bromoacetyl bromide (25.0 g, 125.09 mmol). The mixture was stirred for overnight, evaporated and purified by SiO₂ column chromatography (H₂O/CH₃CN 5:95) to afforded 2,3-bis(2-bromoacetamido)succinic acid (9.95 g, 76% yield) as light yellow oil. MS ESI m/z calcd for C₈H₁₁Br₂N₂O₆ [M+H]⁺ 388.89. found 388.68.

2,3-bis(2-bromoacetamido)succinic acid (3.50 g, 9.02 mmol) in dichloromethane (80 ml) was added oxalyl dichloride (5.80 g, 46.05 mmol) and DMF (0.01 ml). The mixture was stirred for 2.5 h, diluted with toluene, concentrated and co-evaporated with dichloroethane (2×20 ml) and toluene (2×15 ml) to dryness to afford 2,3-bis(2-bromoacetamido)succinyl dichloride as crude product (which is not stable) for the next step without further purification (3.90 g, 102% yield). MS ESI m/z calcd for C₈H9Br₂C1₂N₂O₄ [M+H]⁺ 424.82. found 424.90.

### Example 68. Synthesis of 2,3-bis(((benzyloxy)carbonyl)amino)succinic Acid

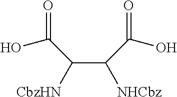 [SMILES: O=C(NC(C(=O)O)C(NC(=O)OCc1ccccc1)C(=O)O)OCc1ccccc1]

To a solution of 2,3-diaminosuccinic acid (4.05 g, 27.35 mmol) in the mixture of THF (250 ml) and NaH₂PO₄ (0.1 M, 250 ml, pH 8.0) was added benzyl carbonochloridate (15.0 g, 88.23 mmol) in 4 portions in 2 h. The mixture was stirred for another 6 h, concentrated and purified on SiO₂ column eluted with H₂O/CH₃CN (1:9) containing 1% formic acid to afford the title compound (8.65 g, 76% yield, ˜95% pure). MS ESI m/z calcd for C₂₀H₂₁N₂O₈ [M+H]⁺ 417.12. found 417.60.

### Example 69. Synthesis of bis(2,5-dioxopyrrolidin-1-yl) 2,3-bis(((benzyloxy)carbonyl)-amino)succinate

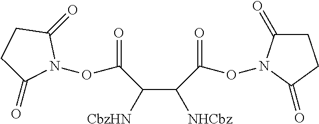 [SMILES: O=C(NC(C(=O)ON1C(=O)CCC1=O)C(NC(=O)OCc1ccccc1)C(=O)ON1C(=O)CCC1=O)OCc1ccccc1]

To a solution of 2,3-bis(((benzyloxy)carbonyl)amino)succinic acid (4.25 g, 10.21 mmol) in the mixture of DMA (70 ml) was added NHS (3.60 g, 31.30 mmol) and EDC (7.05 g, 36.72 mmol). The mixture was stirred for overnight, concentrated and purified on SiO₂ column eluted with EtOAc/CH₂Cl₂ (1:6) to afford the title compound (5.42 g, 87% yield, ˜95% pure). MS ESI m/z calcd for C₂₈H₂₇N₄O₁₂ [M+H]⁺ 611.15. found 611.60.

### Example 70. Synthesis of 2,3-bis(2,5-dioxo-2,5-dihydro-1H-pyrrol-1-yl)succinic Acid

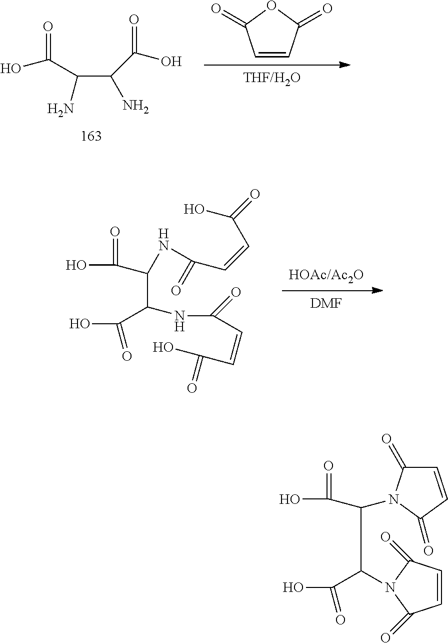

2,3-Diaminosuccinic acid (5.00 g, 33.77 mmol) in the mixture of THF/H₂O/DIPEA (125 ml/125 ml/2 ml) was added maleic anhydride (6.68 g, 68.21 mmol). The mixture was stirred for overnight, evaporated to afforded 2,3-bis((Z)-3-carboxyacrylamido)succinic acid (11.05 g, 99% yield) as a white solid. MS ESI m/z calcd for C₁₂H₁₃N₂O₁₀ [M+H]⁺ 345.05. found 345.35.

2,3-bis((Z)-3-carboxyacrylamido)succinic acid (11.05 g, 33.43 mmol) in a mixture solution of HOAc (70 ml), DMF (10 ml) and toluene (50 ml) was added acetic anhydride (30 ml). The mixture was stirred for 2 h, reflux with Dean-Stark Trap at 100° C. for 6 h, concentrated, co-evaporated with EtOH (2×40 ml) and toluene (2×40 ml), and purified on SiO₂ column eluted with H₂O/CH₃CN (1:10) to afford the title compound (7.90 g, 76% yield, ˜95% pure). MS ESI m/z calcd for C1₂H₉N₂O₈[M+H]⁺ 309.03. found 309.30.

### Example 71. Synthesis of bis(2,5-dioxopyrrolidin-1-yl) 2,3-bis(2,5-dioxo-2,5-dihydro-1H-pyrrol-1-yl)succinate

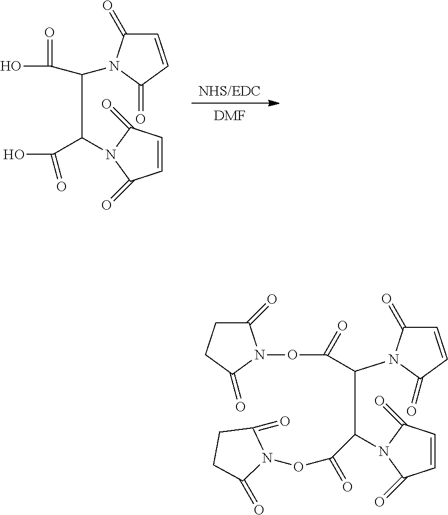

To a solution of 2,3-bis(2,5-dioxo-2,5-dihydro-1H-pyrrol-1-yl)succinic acid (4.00 g, 12.98 mmol) in the mixture of DMF (70 ml) was added NHS (3.60 g, 31.30 mmol) and EDC (7.05 g, 36.72 mmol). The mixture was stirred for overnight, concentrated and purified on SiO₂ column eluted with EtOAc/CH₂Cl₂ (1:6) to afford the title compound (5.73 g, 88% yield, ˜96% pure by HPLC). MS ESI m/z calcd for C₂₀H₁₅N₄O₁₂ [M+H]⁺ 503.06. found 503.45.

### Example 72. Synthesis of (3S,6S,39S,42S)-di-tert-butyl 6,39-bis(4-((tert-butoxycarbonyl)amino)butyl)-22,23-bis(2,5-dioxo-2,5-dihydro-1H-pyrrol-1-yl)-3,42-bis((4-(hydroxymethyl)phenyl)carbamoyl)-5,8,21,24,37,40-hexaoxo-11,14,17,28,31,34-hexaoxa-4,7,20,25,38,41-hexaazatetratetracontane-1,44-dioate

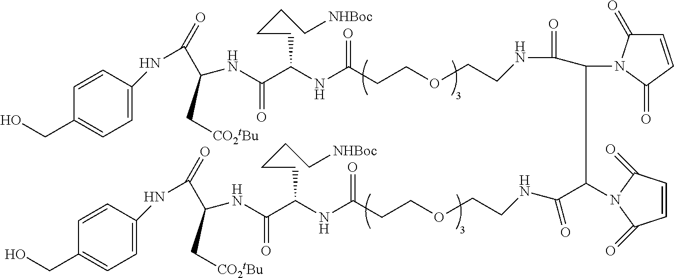

(14S,17 S)-tert-butyl 1-amino-14-(4-((tert-butoxycarbonyl)amino)butyl)-17-((4-(hydroxymethyl)phenyl)carbamoyl)-12,15-dioxo-3,6,9-trioxa-13,16-diazanonadecan-19-oate (1.43 g, 1.97 mmol) and 2,3-bis(2,5-dioxo-2,5-dihydro-1H-pyrrol-1-yl)succinic acid (0.30 g, 0.97 mmol) in DMA (25 ml) was added EDC (1.30 g, 6.77 mmol). The mixture was stirred for overnight, evaporated in vacuo, purified on silica gel using a mixture of methanol (from 5% to 8%) in methylene chloride containing as the eluant to give title compound (1.33 g, 80% yield). ESI MS m/z C₈₂H₁₂₃N₁₂O₂₈ [M+H]⁺, cacld. 1722.85. found 1722.98.

### Example 73. Synthesis of tert-butyl 1-azido-14,17-dimethyl-12,15-dioxo-3,6,9-trioxa-13,16-diazaoctadecan-18-oate

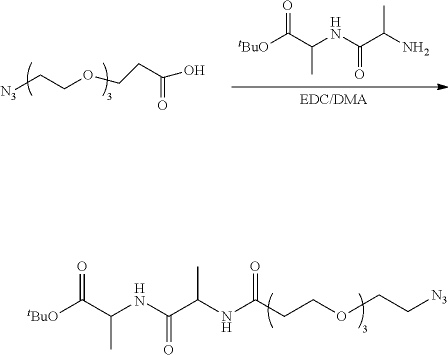

To a solution of 3-(2-(2-(2-azidoethoxy)ethoxy)ethoxy)propanoic acid (1.55 g, 6.27 mmol), tert-butyl 2-(2-aminopropanamido)propanoate (1.35 g, 6.27 mmol) in the mixture of DMA (60 ml) was added EDC (3.05 g, 15.88 mmol). The mixture was stirred for overnight, concentrated and purified on SiO₂ column eluted with EtOAc/CH₂Cl₂ (1:3) to afford the title compound (2.42 g, 86% yield, ˜95% pure by HPLC). MS ESI m/z calcd for C₁₉H3₆N₅O₇ [M+H]⁺ 446.25, found 446.60.

### Example 74. Synthesis of 1-azido-14,17-dimethyl-12,15-dioxo-3,6,9-trioxa-13,16-diazaoctadecan-18-oic Acid

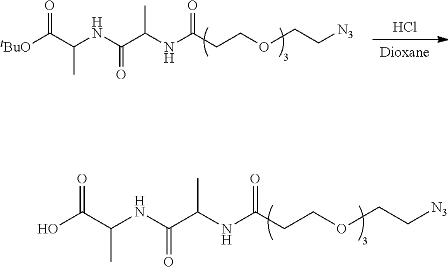

Tert-butyl 1-azido-14,17-dimethyl-12,15-dioxo-3,6,9-trioxa-13,16-diazaoctadecan-18-oate (2.20 g, 4.94 mmol) in 1,4-dioxane (40 ml) was added HCl (12 M, 10 ml). The mixture was stirred for 40 min, diluted with dioxane (20 ml) and toluene (40 ml), evaporated and co-evaporated with dioxane (20 ml) and toluene (40 ml) to dryness to afford the crude title product for the next step without further production (1.92 g, 100% yield, ˜94% pure by HPLC). MS ESI m/z calcd for C₁₅H₂₈N₅O₇ [M+H]⁺ 390.19. found 390.45.

### Example 75. Synthesis of 21,22-bis(2,5-dioxo-2,5-dihydro-1H-pyrrol-1-yl)-2,5,38,41-tetramethyl-4,7,20,23,36,39-hexaoxo-10,13,16,27,30,33-hexaoxa-3,6,19,24,37,40-hexaazadotetracontane-1,42-dioic Acid

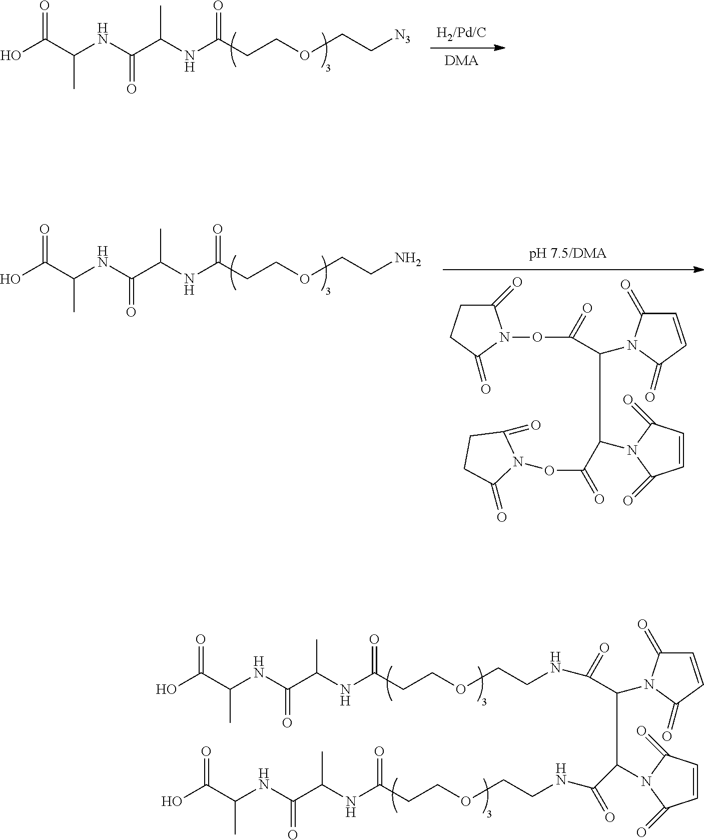

1-azido-14,17-dimethyl-12,15-dioxo-3,6,9-trioxa-13,16-diazaoctadecan-18-oic acid (1.90 g, 4.88 mmol) in DMA (40 ml) was added Pd/C (0.20 g, 50% wet). The system was evacuated under vacuum and placed under 2 atm of hydrogen gas via hydrogenation reactor with vigorous stirring. The reaction was then stirred for 6 h at room temperature and TLC showed that the starting materials disappeared. The crude reaction was passed through a short pad of Celite rinsing with ethanol. The solvent was concentrated under reduced pressure to afford the crude product, 1-amino-14,17-dimethyl-12,15-dioxo-3,6,9-trioxa-13,16-diazaoctadecan-18-oic acid in DMA which was used for the next step directly. ESI MS m/z+ C₁₅H3₀N₃O₇ (M+H), cacld. 364.20. found 364.30.

To the amino compound in DMA (˜30 ml) was added 0.1 M NaH2PO4, pH 7.5 (20 ml), followed by addition of bis(2,5-dioxopyrrolidin-1-yl) 2,3-bis(2,5-dioxo-2,5-dihydro-1H-pyrrol-1-yl)succinate (1.30 g, 2.59 mmol). The mixture was stirred overnight, concentrated and purified on SiO₂ column eluted with 8% water on CH₃CN to afford the title compound (1.97 g, 81% yield). ESI MS m/z+ C₄₂H₆₃N₈O₂₀ (M+H), cacld. 999.41. found 999.95.

### Example 76. Synthesis of bis(2,5-dioxopyrrolidin-1-yl) 21,22-bis(2,5-dioxo-2,5-dihydro-1H-pyrrol-1-yl)-2,5,38,41-tetramethyl-4,7,20,23,36,39-hexaoxo-10,13,16,27,30,33-hexaoxa-3,6,19,24,37,40-hexaazadotetracontane-1,42-dioate

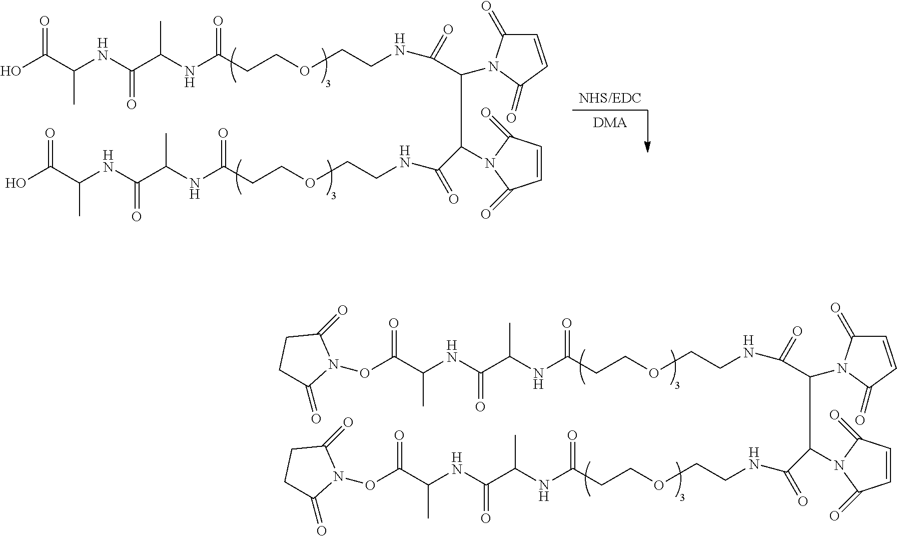

To a solution of 21,22-bis(2,5-dioxo-2,5-dihydro-1H-pyrrol-1-yl)-2,5,38,41-tetramethyl-4,7,20,23,36,39-hexaoxo-10,13,16,27,30,33-hexaoxa-3,6,19,24,37,40-hexaazadotetracontane-1,42-dioic acid (1.50 g, 1.50 mmol) in the mixture of DMA (10 ml) was added NHS (0.60 g, 5.21 mmol) and EDC (1.95 g, 10.15 mmol). The mixture was stirred for overnight, concentrated and purified on SiO₂ column eluted with EtOAc/CH₂Cl₂ (1:4 to 2:1) to afford the title compound (1.50 g, 83% yield, ˜95% pure by HPLC). MS ESI m/z calcd for C₅₀H₆₉N₁₀O₂₄ [M+H]⁺ 1193.44. found 1193.95.

### Example 77. Synthesis of (S)-tert-butyl 2-(hydroxymethyl)pyrrolidine-1-carboxylate

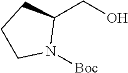 [SMILES: CC(C)(C)OC(=O)N1CCC[C@H]1CO]

Boc-L-proline (10.0 g, 46.4 mmol) dissolved in 50 mL THF was cooled to 0° C., to which BH₃ in THF (1.0 M, 46.4 mL) was added carefully. The mixture was stirred at 0° C. for 1.5 h then poured onto ice water and extracted with ethyl acetate. The organic layer was washed with brine (50 mL), dried over anhydrous Na₂SO₄, and concentrated under reduced pressure to give the title compound (8.50 g, 91% yield) as a white solid. ¹H NMR (500 MHz, CDCl₃) δ 3.94 (dd, J=4.9, 2.7 Hz, 2H), 3.60 (ddd, J=18.7, 11.9, 9.3 Hz, 2H), 3.49-3.37 (m, 1H), 3.34-3.23 (m, 1H), 2.06-1.91 (m, 1H), 1.89-1.69 (m, 2H), 1.65-1.51 (m, 1H), 1.49-0.40 (m, 9H).

### Example 78. Synthesis of (S)-tert-butyl 2-formylpyrrolidine-1-carboxylate

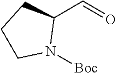 [SMILES: CC(C)(C)OC(=O)N1CCC[C@H]1C=O]

To a solution of (S)-tert-butyl 2-(hydroxymethyl)pyrrolidine-1-carboxylate (13.0 g, 64.6 mmol) in dimethyl sulfoxide (90 mL) was added triethylamine (40 mL) and the stirring was continued for 15 min. The mixture was cooled over ice bath and sulfur trioxide-pyridine complex (35.98 g, 226 mmol) was added in portions over a 40 min period. The reaction was warmed to r.t. and stirred for 2.5 h. After addition of ice (250 g), the mixture was extracted with dichloromethane (150 mL×3). The organic phase was washed with 50% citric acid solution (150 mL), water (150 mL), saturated sodium bicarbonate solution (150 mL), and brine (150 mL), dried over anhydrous Na₂SO₄. Removal of solvent in vacuo yielded the title aldehyde (10.4 g, 81% yield) as a dense oil which was used without further purification. ¹H NMR (500 MHz, CDCl₃) δ 9.45 (s, 1H), 4.04 (s, 1H), 3.53 (dd, J=14.4, 8.0 Hz, 2H), 2.00-1.82 (m, 4H), 1.44 (d, J=22.6 Hz, 9H).

### Example 79. Synthesis of (4R,5S)-4-methyl-5-phenyl-3-propionyloxazolidin-2-one

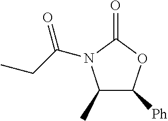 [SMILES: CCC(=O)N1C(=O)O[C@@H](c2ccccc2)[C@H]1C]

n-Butyllithium in hexane (21.6 mL, 2.2 M, 47.43 mmol) was added dropwise at −78° C. to a stirred solution of 4-methyl-5-phenyloxazolidin-2-one (8.0 g, 45.17 mmol) in THF (100 mL) under N₂. The solution was maintained at −78° C. for 1 h then propionyl chloride (4.4 mL, 50.59 mmol) was added slowly. The reaction mixture was warmed to −50 OC, stirred for 2 h then quenched by addition of a saturated solution of ammonium chloride (100 mL). The organic solvent was removed in vacuo and the resultant solution was extracted with ethyl acetate (3×100 mL). The organic layer was washed with saturated sodium bicarbonate solution (100 mL) and brine (100 mL), dried over Na₂SO₄, filtered and concentrated in vacuo. The residue was purified by column chromatography (20% ethyl acetate/hexanes) to afford the title compound as a dense oil (10.5 g, 98% yield). 1H NMR (500 MHz, CDCl₃) δ 7.45-7.34 (m, 3H), 7.30 (d, J=7.0 Hz, 2H), 5.67 (d, J=7.3 Hz, 1H), 4.82-4.70 (m, 1H), 2.97 (dd, J=19.0, 7.4 Hz, 2H), 1.19 (t, J=7.4 Hz, 3H), 0.90 (d, J=6.6 Hz, 3H).

### Example 80. Synthesis of (S)-tert-butyl 2-((1R,2R)-1-hydroxy-2-methyl-3-((4R,5S)-4-methyl-2-oxo-5-phenyloxazolidin-3-yl)-3-oxopropyl)pyrrolidine-1-carboxylate

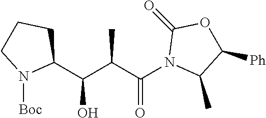 [SMILES: C[C@@H]1[C@H](c2ccccc2)OC(=O)N1C(=O)[C@H](C)[C@@H](O)[C@@H]1CCCN1C(=O)OC(C)(C)C]

To a solution of (4R,5S)-4-methyl-5-phenyl-3-propionyloxazolidin-2-one (9.40 g, 40.4 mmol) in dichloromethane (60 mL) was added Et₃N (6.45 mL, 46.64 mmol) at 0° C., followed by 1M dibutylboron triflate in dichloromethane (42 mL, 42 mmol). The mixture was stirred at 0° C. for 45 min, cooled to −70° C., (S)-tert-butyl 2-formylpyrrolidine-1-carboxylate (4.58 g, 22.97 mmol) in dichloromethane (40 mL) was then added slowly over a 30 min period. The reaction was stirred at −70° C. for 2 h, 0° C. 1 h, and r.t. 15 min, and then quenched with phosphate buffer solution (pH 7, 38 mL). After the addition of MeOH-30% H₂O₂ (2:1, 100 mL) at below 10° C. and stirring for 20 min, water (100 mL) was added and the mixture was concentrated in vacuo. More water (200 mL) was added to the residue and the mixture was extracted with ethyl acetate (3×100 mL). The organic layer was washed with 1N KHSO₄ (100 mL), sodium bicarbonate solution (100 mL) and brine (100 mL), dried over anhydrous Na₂SO₄ and concentrated in vacuo. The residue was purified by flash column chromatography (10%-50% ethyl acetate/hexanes) to afford the title compound as a white solid (7.10 g, 71% yield). ¹H NMR (500 MHz, CDCl₃) δ 7.39 (dt, J=23.4, 7.1 Hz, 3H), 7.30 (d, J=7.5 Hz, 2H), 5.67 (d, J=7.1 Hz, 1H), 4.84-4.67 (m, 1H), 4.08-3.93 (m, 3H), 3.92-3.84 (m, 1H), 3.50 (d, J=9.0 Hz, 1H), 3.24 (d, J=6.7 Hz, 1H), 2.15 (s, 1H), 1.89 (dd, J=22.4, 14.8 Hz, 3H), 1.48 (d, J=21.5 Hz, 9H), 1.33 (d, J=6.9 Hz, 3H), 0.88 (d, J=6.4 Hz, 3H).

### Example 81. Synthesis of (S)-tert-butyl 2-((1R,2R)-1-methoxy-2-methyl-3-((4R,5S)-4-methyl-2-oxo-5-phenyloxazolidin-3-yl)-3-oxopropyl)pyrrolidine-1-carboxylate

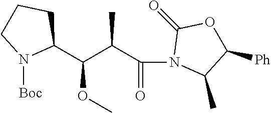 [SMILES: CO[C@H]([C@@H](C)C(=O)N1C(=O)O[C@@H](c2ccccc2)[C@H]1C)[C@@H]1CCCN1C(=O)OC(C)(C)C]

To a mixture of (S)-tert-butyl 2-((1R,2R)-1-hydroxy-2-methyl-3-((4R,5 S)-4-methyl-2-oxo-5-phenyloxazolidin-3-yl)-3-oxopropyl)pyrrolidine-1-carboxylate (5.1 g 11.9 mmol) and molecular sieves (4 Å, 5 g) was added anhydrous dichloroethane (30 mL) under N₂. The mixture was stirred at room temperature for 20 min and cooled to 0° C. Proton sponge (6.62 g, 30.9 mmol) was added, followed by trimethyloxonium tetrafluoroborate (4.40 g, 29.7 mmol). Stirring was continued for 2 h at 0° C. and 48 h at r.t. The reaction mixture was filtrated and the filtrate was concentrated and purified by column chromatography (20-70% ethyl acetate/hexanes) to afford the title compound as a white solid (1.80 g, 35% yield). ¹H NMR (500 MHz, CDCl₃) δ 7.46-7.27 (m, 5H), 5.65 (s, 1H), 4.69 (s, 1H), 3.92 (s, 1H), 3.83 (s, 1H), 3.48 (s, 3H), 3.17 (s, 2H), 2.02-1.68 (m, 5H), 1.48 (d, J=22.3 Hz, 9H), 1.32 (t, J=6.0 Hz, 3H), 0.91-0.84 (m, 3H).

### Example 82. Synthesis of (2R,3R)-3-((S)-1-(tert-butoxycarbonyl)pyrrolidin-2-yl)-3-methoxy-2-methylpropanoic Acid

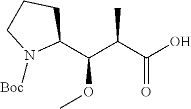 [SMILES: CO[C@H]([C@@H](C)C(=O)O)[C@@H]1CCCN1C(=O)OC(C)(C)C]

To a solution of (S)-tert-butyl 2-((1R,2R)-1-methoxy-2-methyl-3-((4R,5S)-4-methyl-2-oxo-5-phenyloxazolidin-3-yl)-3-oxopropyl)pyrrolidine-1-carboxylate (1.80 g, 4.03 mmol) in THF (30 mL) and H₂O (7.5 mL), 30% H₂O₂ (1.44 mL, 14.4 mmol) was added over a 5 min period at 0° C., followed by a solution of LiOH (0.27 g, 6.45 mmol) in water (5 mL). After stirring at 0° C. for 3 h, 1 N sodium sulfite (15.7 mL) was added and the mixture was allowed to warm to r.t. and stirred overnight. THF was removed in vacuo and the aqueous phase was wash with dichloromethane (3×50 mL) to remove the oxazolidinone auxiliary. The aqueous phase was acidified to pH 3 with 1N HCl and extracted with ethyl acetate (3×50 mL). The organic layer was washed with brine (50 mL), dried over Na2SO4, filtered and concentrated in vacuo to afford the title compound as a colorless oil (1.15 g, 98% yield). ¹H NMR (500 MHz, CDCl₃) δ 3.99-3.74 (m, 2H), 3.44 (d, J=2.6 Hz, 3H), 3.23 (s, 1H), 2.60-2.45 (m, 1H), 1.92 (tt, J=56.0, 31.5 Hz, 3H), 1.79-1.69 (m, 1H), 1.58-1.39 (m, 9H), 1.30-1.24 (m, 3H).

### Example 83. Synthesis of (2R,3R)-methyl 3-methoxy-2-methyl-3-((S)-pyrrolidin-2-yl)propanoate

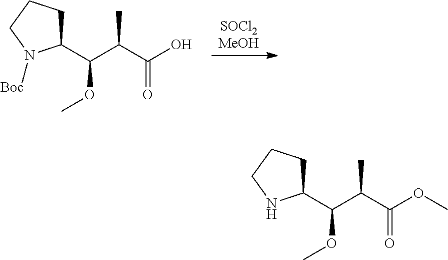 [SMILES: CO[C@H]([C@@H](C)C(=O)O)[C@@H]1CCCN1C(=O)OC(C)(C)C ; COC(=O)[C@H](C)[C@@H](OC)[C@@H]1CCCN1 ; "SOCl2MeOH"]

To a solution of (2R,3R)-3-((S)-1-(tert-butoxycarbonyl)pyrrolidin-2-yl)-3-methoxy-2-methylpropanoic acid. (0.86 g, 2.99 mmol) in MeOH (10 mL) was added thionyl chloride (1.08 mL, 14.95 mmol) slowly at 0° C. The reaction was then warmed to room temperature and stirred overnight. The mixture was concentrated in vacuo and co-evaporation with toluene giving the title compound (0.71 g, 100% yield) as a white solid, which was immediately used for the next step without further purification. HRMS (ESI) m/z calcd. for C₁₀H₂₀NO₃ [M+H]⁺: 202.14. found: 202.14.

### Example 84. Synthesis of (4S,5S)-ethyl 4-((tert-butoxycarbonyl)amino)-5-methyl-3-oxo heptanoate

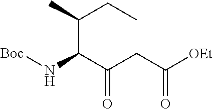 [SMILES: CCOC(=O)CC(=O)[C@@H](NC(=O)OC(C)(C)C)[C@@H](C)CC]

To an ice-cooled solution of N-Boc-L-isoleucine (4.55 g, 19.67 mmol) in THF (20 mL) was added 1,1′-carbonyldiimidazole (3.51 g, 21.63 mmol). After evolution of gas ceased, the resultant mixture was stirred at r.t. for 3.5 h.

A solution of freshly prepared isopropylmagnesium bromide in THF (123 mmol, 30 mL) was added dropwise to a pre-cooled (0° C.) solution of ethyl hydrogen malonate (6.50 g, 49.2 mmol) at such a rate to keep the internal temperature below 5° C. The mixture was stirred at r.t. for 1.5 h. This solution of the magnesium enolate was then cooled over an ice-water bath, followed by the gradual addition of the imidazolide solution over a 1 h period via a double-ended needle at 0° C. The resultant mixture was stirred at 0° C. for 30 min then r.t. 64 h. The reaction mixture was quenched by addition of 10% aqueous citric acid (5 mL), and acidified to pH 3 with an additional 10% aqueous citric acid (110 mL). The mixture was extracted with ethyl acetate (3×150 mL). The organic extracts were washed with water (50 mL), saturated aqueous sodium hydrogen carbonate (50 mL), and saturated aqueous sodium chloride (50 mL), dried over Na₂SO₄, and concentrated in vacuo. The residue was purified by column chromatography on silica gel using ethyl acetate/hexane (1:4) as an eluent to give the title compound (5.50 g, 93% yield). ¹H NMR (500 MHz, CDCl₃) δ 5.04 (d, J=7.8 Hz, 1H), 4.20 (p, J=7.0 Hz, 3H), 3.52 (t, J=10.7 Hz, 2H), 1.96 (d, J=3.7 Hz, 1H), 1.69 (s, 2H), 1.44 (s, 9H), 1.28 (dd, J=7.1, 2.9 Hz, 3H), 0.98 (t, J=6.9 Hz, 3H), 0.92-0.86 (m, 3H).

### Example 85. Synthesis of (3R,4S,5S)-ethyl 4-((tert-butoxycarbonyl)amino)-3-hydroxy-5-methylheptanoate

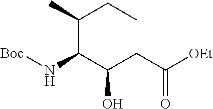 [SMILES: CCOC(=O)C[C@@H](O)[C@@H](NC(=O)OC(C)(C)C)[C@@H](C)CC]

To a solution of (4S,5S)-ethyl 4-((tert-butoxycarbonyl)amino)-5-methyl-3-oxo heptanoate (5.90 g, 19.83 mmol) in ethanol (6 mL) at −60 OC was added sodium borohydride (3.77 g, 99.2 mmol) in one portion. The reaction mixture was stirred for 5.5 h below −55° C. then quenched with 10% aqueous citric acid (100 mL). The resultant solution was acidified to pH 2 with an additional 10% aqueous citric acid, followed by extraction with ethyl acetate (3×100 mL). The organic extracts were washed with saturated aqueous sodium chloride (100 mL), dried over Na₂SO₄, and concentrated in vacuo. The residue was purified by column chromatography (10-50% ethyl acetate/hexane) to give pure the title compound as diastereomer (2.20 g, 37% yield) and a mixture of two diastereomers (2.0 g, 34% yield, about 9:1 ratio). ¹H NMR (500 MHz, CDCl₃) δ 4.41 (d, J=9.3 Hz, 1H), 4.17 (tt, J=7.1, 3.6 Hz, 2H), 4.00 (t, J=6.9 Hz, 1H), 3.55 (dd, J=11.7, 9.3 Hz, 1H), 2.56-2.51 (m, 2H), 2.44 (dd, J=16.4, 9.0 Hz, 1H), 1.79 (d, J=3.8 Hz, 1H), 1.60-1.53 (m, 1H), 1.43 (s, 9H), 1.27 (dd, J=9.3, 5.0 Hz, 3H), 1.03-0.91 (m, 7H).

### Example 86. Synthesis of (3R,4S,5S)-4-((tert-butoxycarbonyl)amino)-3-hydroxy-5-methyl Heptanoic Acid

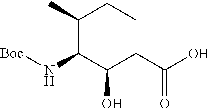 [SMILES: CC[C@H](C)[C@H](NC(=O)OC(C)(C)C)[C@H](O)CC(=O)O]

To a solution of (3R,4S,5S)-ethyl 4-((tert-butoxycarbonyl)amino)-3-hydroxy-5-methylheptanoate (2.20 g, 7.20 mmol) in ethanol (22 mL) was added 1 N aqueous sodium hydroxide (7.57 mL, 7.57 mmol). The mixture was stirred at 0° C. for 30 min then r.t. 2 h. The resultant solution was acidified to pH 4 by addition of 1 N aqueous hydrochloric acid, which was then extracted with ethyl acetate (3×50 mL). The organic extracts were washed with 1 N aqueous potassium hydrogen sulfate (50 mL), and saturated aqueous sodium chloride (50 mL), dried over Na₂SO₄, and concentrated in vacuo to give the compound (1.90 g, 95% yield). ¹H NMR (500 MHz, CDCl₃) δ 4.50 (d, J=8.7 Hz, 1H), 4.07 (d, J=5.5 Hz, 1H), 3.59 (d, J=8.3 Hz, 1H), 2.56-2.45 (m, 2H), 1.76-1.65 (m, 1H), 1.56 (d, J=7.1 Hz, 1H), 1.45 (s, 9H), 1.26 (t, J=7.1 Hz, 3H), 0.93 (dd, J=14.4, 7.1 Hz, 6H).

### Example 87. Synthesis of (3R,4S,5S)-4-((tert-butoxycarbonyl)(methyl)amino)-3-methoxy-5-methylheptanoic Acid

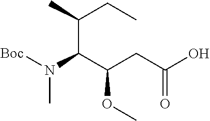 [SMILES: CC[C@H](C)[C@@H]([C@@H](CC(=O)O)OC)N(C)C(=O)OC(C)(C)C]

To a solution of (3R,4S,5S)-4-((tert-butoxycarbonyl)amino)-3-hydroxy-5-methyl heptanoic acid (1.90 g, 6.9 mmol) in THF (40 mL) was added sodium hydride (60% oil suspension, 1.93 g, 48.3 mmol) at 0° C. After stirring for 1 h, methyl iodide (6.6 mL, 103.5 mmol) was added. The stirring was continued at 0° C. for 40 h before saturated aqueous sodium hydrogen carbonate (50 mL) was added, followed by water (100 mL). The mixture was washed with diethyl ether (2×50 mL) and the aqueous layer was acidified to pH 3 by 1 N aqueous potassium hydrogen sulfate, then extracted with ethyl acetate (3×50 mL). The combined organic extracts were washed with 5% aqueous sodium thiosulfate (50 mL) and saturated aqueous sodium chloride (50 mL), dried over Na₂SO₄, and concentrated in vacuo to give the title compound (1.00 g, 48% yield). ¹H NMR (500 MHz, CDCl₃) δ 3.95 (d, J=75.4 Hz, 2H), 3.42 (d, J=4.4 Hz, 3H), 2.71 (s, 3H), 2.62 (s, 1H), 2.56-2.47 (m, 2H), 1.79 (s, 1H), 1.47 (s, 1H), 1.45 (d, J=3.3 Hz, 9H), 1.13-1.05 (m, 1H), 0.96 (d, J=6.7 Hz, 3H), 0.89 (td, J=7.2, 2.5 Hz, 3H).

### Example 88. Synthesis of Boc-N-Me-L-Val-OH

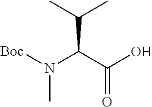 [SMILES: CC(C)[C@@H](C(=O)O)N(C)C(=O)OC(C)(C)C]

To a solution of Boc-L-Val-OH (2.00 g, 9.2 mmol) and methyl iodide (5.74 mL, 92 mmol) in anhydrous THF (40 mL) was added sodium hydride (3.68 g, 92 mmol) at 0° C. The reaction mixture was stirred at 0° C. for 1.5 h, then warmed to r.t. and stirred for 24 h. The reaction was quenched by ice water (50 mL). After addition of water (100 mL), the reaction mixture was washed with ethyl acetate (3×50 mL) and the aqueous solution was acidified to pH 3 then extracted with ethyl acetate (3×50 mL). The combined organic phase was dried over Na₂SO₄ and concentrated to afford Boc-N-Me-Val-OH (2.00 g, 94% yield) as a white solid. ¹H NMR (500 MHz, CDCl₃) δ 4.10 (d, J=10.0 Hz, 1H), 2.87 (s, 3H), 2.37-2.13 (m, 1H), 1.44 (d, J=26.7 Hz, 9H), 1.02 (d, J=6.5 Hz, 3H), 0.90 (t, J=8.6 Hz, 3H).

### Example 89. Synthesis of (2R,3R)-methyl 3-((S)-1-((3R,4S,5S)-4-((tert-butoxycarbonyl)-(methyl)amino)-3-methoxy-5-methylheptanoyl)pyrrolidin-2-yl)-3-methoxy-2-methylpropanoate

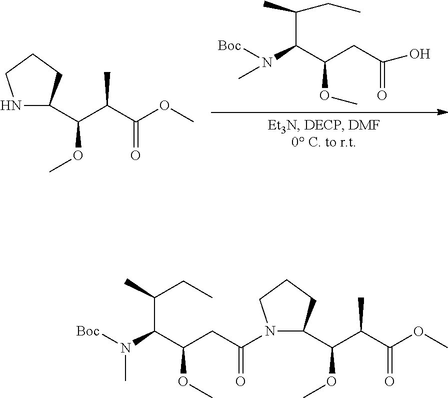

To a solution of (2R,3R)-methyl 3-methoxy-2-methyl-3-((S)-pyrrolidin-2-yl)propanoate (0.71 g, 2.99 mmol) and (3R,4S,5S)-4-((tert-butoxycarbonyl)(methyl)amino)-3-methoxy-5-methylheptanoic acid (1 g, 3.29 mmol) in DMF (10 mL) at 0° C. was added diethyl cyanophosphonate (545 μL, 3.59 mmol), followed by addition of Et₃N (1.25 mL, 8.99 mmol). The reaction mixture was stirred at 0° C. for 2 h, then warmed to room temperature and stirred overnight. The reaction mixture was diluted with ethyl acetate (50 mL), washed with 1 N aqueous potassium hydrogen sulfate (20 mL), water (20 mL), saturated aqueous sodium hydrogen carbonate (20 mL), and saturated aqueous sodium chloride (20 mL), dried over sodium sulfate, and concentrated in vacuo. The residue was purified on silica gel column chromatography eluted with ethyl acetate/hexane (1:5 to 2:1) to afford the title (0.9 g, 62% yield) as a white solid. HRMS (ESI) m/z calcd. for C₂₅H₄₆N₂O₇ [M+H]+: 487.33, found: 487.32.

### Example 90. Synthesis of (S)-tert-butyl 2-((1R,2R)-1-methoxy-3-(((S)-1-methoxy-1-oxo-3-phenylpropan-2-yl)amino)-2-methyl-3-oxopropyl)pyrrolidine-1-carboxylate

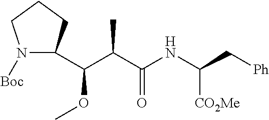 [SMILES: COC(=O)[C@H](Cc1ccccc1)NC(=O)[C@H](C)[C@@H](OC)[C@@H]1CCCN1C(=O)OC(C)(C)C]

To a solution of (2R,3R)-3-((S)-1-(tert-butoxycarbonyl)pyrrolidin-2-yl)-3-methoxy-2-methylpropanoic acid (100 mg, 0.347 mmol) and L-phenylalanine methyl ester hydrochloride (107.8 mg, 0.500 mmol) in DMF (5 mL) at 0° C. was added diethyl cyanophosphonate (75.6 μL, 0.451 mmol), followed by Et₃N (131 μL, 0.94 mmol). The reaction mixture was stirred at 0° C. for 2 h, then warmed to r.t. and stirred overnight. The reaction mixture was then diluted with ethyl acetate (80 mL), washed with 1 N aqueous potassium hydrogen sulfate (40 mL), water (40 mL), saturated aqueous sodium hydrogen carbonate (40 mL), and saturated aqueous sodium chloride (40 mL), dried over Na₂SO₄, and concentrated in vacuo. The residue was purified by column chromatography (15-75% ethyl acetate/hexanes) to afford the title compound (130 mg, 83% yield) as a white solid. ¹H NMR (500 MHz, CDCl₃) δ 7.28 (dd, J=7.9, 6.5 Hz, 2H), 7.23 (t, J=7.3 Hz, 1H), 7.16 (s, 2H), 4.81 (s, 1H), 3.98-3.56 (m, 5H), 3.50 (s, 1H), 3.37 (d, J=2.9 Hz, 3H), 3.17 (dd, J=13.9, 5.4 Hz, 2H), 3.04 (dd, J=14.0, 7.7 Hz, 1H), 2.34 (s, 1H), 1.81-1.69 (m, 2H), 1.65 (s, 3H), 1.51-1.40 (m, 9H), 1.16 (d, J=7.0 Hz, 3H).

### Example 91. General Procedure for the Removal of the Boc Function with Trifluoroacetic Acid

To a solution of the N-Boc amino acid (1.0 mmol) in methylene chloride (2.5 mL) was added trifluoroacetic acid (1.0 mL). After being stirred at room temperature for 1-3 h, the reaction mixture was concentrated in vacuo. Co-evaporation with toluene gave the deprotected product, which was used without any further purification.

### Example 92. Synthesis of (2R,3R)-methyl 3-((S)-1-((3R,4S,5S)-4-((S)-2-((tert-butoxycarbonyl)amino)-N,3-dimethylbutanamido)-3-methoxy-5-methylheptanoyl)pyrrolidin-2-yl)-3-methoxy-2-methylpropanoate

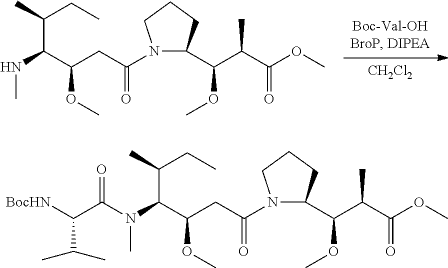

To a solution of the deprotected product from (2R,3R)-methyl 3-methoxy-3-((S)-1-((3R,4S,5S)-3-methoxy-5-methyl-4-(methylamino)heptanoyl)pyrrolidin-2-yl)-2-methylpropanoate (715 mg, 1.85 mmol) and Boc-Val-OH (1.2 g, 5.56 mmol) in DCM (20 mL) at 0° C. was added BroP (1.08 g, 2.78 mmol), followed by addition of diisopropylethylamine (1.13 mL, 6.48 mmol). The mixture was shielded from light and stirred at 0° C. for 30 min then at r.t. for 48 h. The reaction mixture was diluted with ethyl acetate (50 mL), washed with 1 N aqueous potassium hydrogen sulfate (20 mL), water (20 mL), saturated aqueous sodium hydrogen carbonate (20 mL), and saturated aqueous sodium chloride (20 mL), dried over Na₂SO₄ and concentrated in vacuo. The residue was purified on silica gel column chromatography eluted with ethyl acetate/hexane (1:5 to 4:1) to afford the title compound (0.92 g, 85% yield) as a white solid. HRMS (ESI) m/z calcd. for C₃₀H₅₅N₃O₈ [M+H]+: 586.40, found: 586.37.

### Example 93. Synthesis of (2R,3R)-methyl 3-((S)-1-((3R,4S,5S)-4-((S)-2-(2-(dimethylamino)-2-methylpropanamido)-N,3-dimethylbutanamido)-3-methoxy-5-methylheptanoyl)pyrrolidin-2-yl)-3-methoxy-2-methylpropanoate

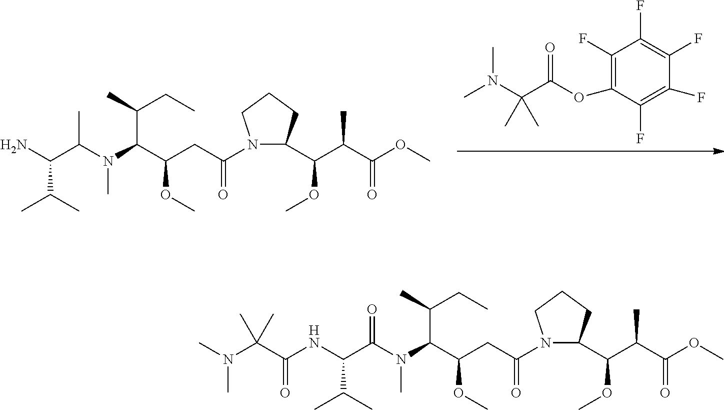

To a solution of the deprotected product from (2R,3R)-methyl 3-((S)-1-((3R,4S,5S)-4-((S)-2-((tert-butoxycarbonyl)amino)-N,3-dimethylbutanamido)-3-methoxy-5-methylheptanoyl)pyrrolidin-2-yl)-3-methoxy-2-methylpropanoate (50 mg, 0.085 mmol) and perfluorophenyl 2-(dimethylamino)-2-methylpropanoate (74.5 mg, 0.25 mmol) in DMF (2 ml) at 0° C. was added DIPEA (44 μL, 0.255 mmol). The reaction mixture was warmed to RT and stirred 2 h. The reaction mixture was diluted with ethyl acetate (30 mL), washed with water (10 mL), and saturated aqueous sodium chloride (10 mL), dried over sodium sulfate, and concentrated in vacuo. The residue was purified on silica gel column chromatography eluted with ethyl acetate/hexane (1:5 to 5:1) to afford the title compound (50 mg, 100% yield). HRMS (ESI) m/z calcd. for C₃₁H₅₈N₄O₇ [M+H]+: 599, found: 599.

### Example 94. Synthesis of (2R,3R)-3-((S)-1-((3R,4S,5S)-4-((S)-2-(2-(dimethylamino)-2-methylpropanamido)-N,3-dimethylbutanamido)-3-methoxy-5-methylheptanoyl)pyrrolidin-2-yl)-3-methoxy-2-methylpropanoic Acid

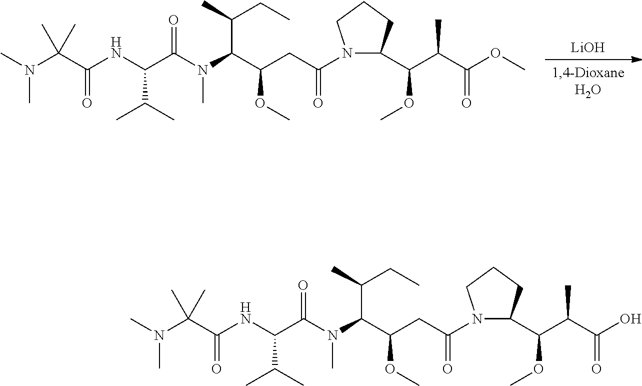

To a solution of (2R,3R)-methyl 3-((S)-1-((3R,4 S,5S)-4-((S)-2-(2-(dimethylamino)-2-methylpropanamido)-N,3-dimethylbutanamido)-3-methoxy-5-methylheptanoyl)pyrrolidin-2-yl)-3-methoxy-2-methylpropanoate (50 mg, 0.0836 mmol) in 1,4-Dioxane (3 mL) at 0-4° C. was added a solution of lithium hydroxide (14 mg, 0.334 mmol) in water (3 mL) drop by drop in 5 min. The reaction mixture was warmed to RT and stirred 2 h. The mixture was acidified to pH 7 with 1N HCl and concentrated under vacuum, and then used for the next step without further purification. HRMS (ESI) m/z calcd. for C₃₀H₅₇N₄O₇ [M+H]+: 585.41, found: 585.80.

### Example 95. Synthesis of (2R,3R)-perfluorophenyl 3-((S)-1-((3R,4S,5S)-4-((S)-2-(2-(dimethylamino)-2-methylpropanamido)-N,3-dimethylbutanamido)-3-methoxy-5-methylheptanoyl)pyrrolidin-2-yl)-3-methoxy-2-methylpropanoate

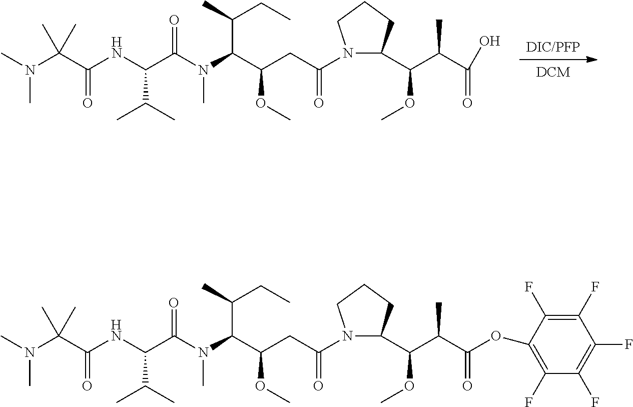

To a solution of (2R,3R)-3-((S)-1-((3R,4S,5S)-4-((S)-2-(2-(dimethylamino)-2-methylpropanamido)-N,3-dimethylbutanamido)-3-methoxy-5-methylheptanoyl)pyrrolidin-2-yl)-3-methoxy-2-methylpropanoic acid (0.0836 mmol) and PFP (18.5 mg, 0.1 mmol) in DCM (2 mL) was added DIC (12.7 mg, 0.1 mmol) at 0° C. The mixture was warmed to RT and stirred overnight. The reaction mixture was concentrated under vacuum and used for the next step without further purification. HRMS (ESI) m/z calcd. for C₃₆H₅₆F₅N₄O₇ [M+H]+: 751.40. found: 751.70.

### Example 96. Synthesis of (S)-methyl 2-((tert-butoxycarbonyl)amino)-3-(4-hydroxy-3-nitrophenyl)propanoate

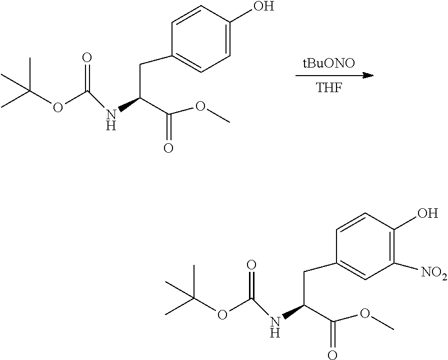 [SMILES: "tBuONO" ; COC(=O)[C@H](Cc1ccc(O)cc1)NC(=O)OC(C)(C)C ; COC(=O)[C@H](Cc1ccc(O)c([N+](=O)[O-])c1)NC(=O)OC(C)(C)C ; "THF"]

To a solution of Boc-L-Tyrosine methyl ester (5 g, 16.9 mmol) in THF (50 mL) was added tert-Butyl nitrite (10 mL, 84.6 mmol), then the reaction mixture was stirred for 5 h at RT. The reaction mixture was concentrated and purified by column chromatography on silica gel using ethyl acetate/hexane (1:10 to 1:5) to afford the compound (4.5 g, 78% yield) as a yellow solid. HRMS (ESI) m/z calcd. for C₁₅H₂₁N₂O₇ [M+H]+: 341.13, found: 341.30.

### Example 97. Synthesis of (S)-methyl 3-(3-amino-4-hydroxyphenyl)-2-((tert-butoxycarbonyl)amino)propanoate

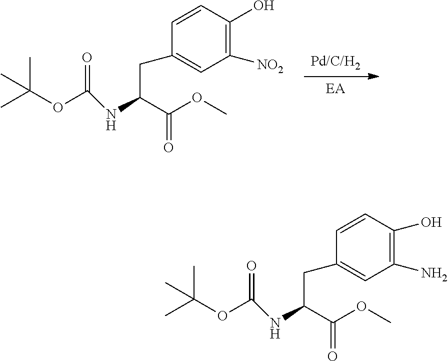 [SMILES: COC(=O)[C@H](Cc1ccc(O)c([N+](=O)[O-])c1)NC(=O)OC(C)(C)C ; "EA" ; "Pd/C/H2" ; COC(=O)[C@H](Cc1ccc(O)c(N)c1)NC(=O)OC(C)(C)C]

To a solution of (S)-methyl 3-(3-amino-4-hydroxyphenyl)-2-(tert-butoxycarbonylamino)propanoate (2 g, 6.44 mmol) in ethyl acetate (20 mL) was added Pd/C (0.2 g) and stirred for 2 h under hydrogen atmosphere. The mixture was filtered and the filtrate was concentrated under vacuum to afford the title compound (1.7 g, 95% yield) as a white solid. HRMS (ESI) m/z calcd. for C₁₅H₂₃N₂O₅ [M+H]+: 311.15, found: 311.30.

### Example 98. Synthesis of Compound A-1

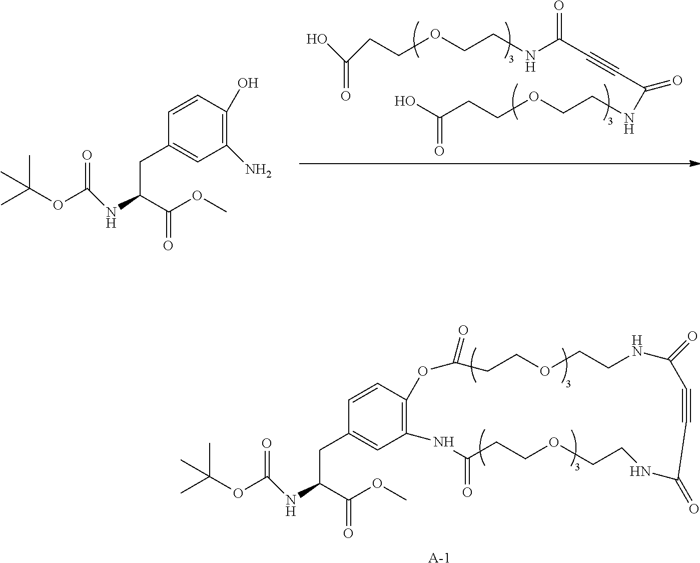

To a solution of 14,17-dioxo-4,7,10,21,24,27-hexaoxa-13,18-diazatriacont-15-yne-1,30-dioic acid (95 mg, 0.182 mmol) and (S)-methyl 3-(3-amino-4-hydroxyphenyl)-2-(tert-butoxycarbonylamino)propanoate (56.6 mg, 0.182 mmol) in DMF (5 mL) at 0° C. was added EDC (128.5 mg, 0.338 mmol), followed by addition of DIPEA (64 μL, 0.365 mmol). The reaction mixture was warmed to rt and stirred overnight. The mixture was diluted with ethyl acetate (30 mL), washed with water (10 mL) and saturated aqueous sodium chloride (10 mL), dried over sodium sulfate and concentrated in vacuo. The residue was purified on silica gel column chromatography eluted with DCM/MeOH (20:1 to 10:1) to afford the compound A-1 (68 mg, 47% yield). HRMS (ESI) m/z calcd. for C₃₇H₅₅N₄O₁₅ [M+H]+: 795.36, found: 795.30.

### Example 99. Synthesis of Compound A-2

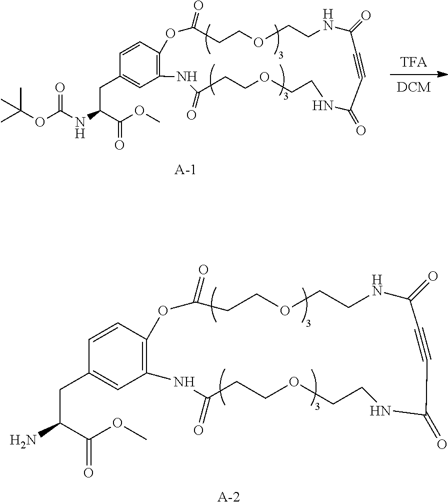

To a solution of compound A-1 (32 mg, 0.04 mmol) in DCM (3 mL) was added TFA (1 mL) at 0° C. The reaction mixture was warmed to RT and stirred 30 min., diluted with toluene, concentrated, co-evaporated with toluene, and then used for the next step without further purification. HRMS (ESI) m/z calcd. for C₃₃H₄₇N₄O₁₅ [M+H]+: 795.36, found: 795.30.

### Example 100. Synthesis of Compound A-3

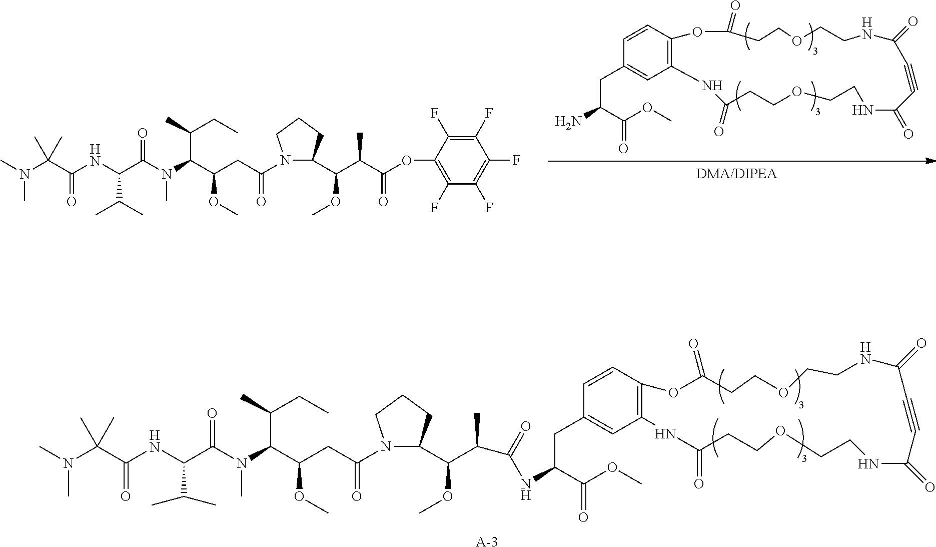

To a solution of (2R,3R)-perfluorophenyl 3-((S)-1-((3R,4S,5S)-4-((S)-2-(2-(dimethylamino)-2-methylpropanamido)-N,3-dimethylbutanamido)-3-methoxy-5-methylheptanoyl)pyrrolidin-2-yl)-3-methoxy-2-methylpropanoate (20 mg, 0.027 mmol) and compound A-2 (31.7 mg, 0.04 mmol) in DMA (2 mL) was added DIPEA (9 μL, 0.053 mmol) at 0° C. The reaction mixture was warmed to RT and stirred for 30 min. The mixture was concentrated under vacuum and purified by prep-HPLC (C-18, 250 mm×10 mm, eluted with H₂O/CH₃CN (9 ml/min, from 90% water to 40% water in 40 min) to afford the compound A-3 (14 mg, 42% yield). HRMS (ESI) m/z calcd. for C₆₂H₁₀₁N₈O₁₉ [M+H]+: 1261.71 found: 1261.30.

### Example 101. Synthesis of (S)-methyl 2-((2R,3R)-3-((S)-1-((3R,4S,5S)-4-((tert-butoxycarbonyl)(methyl)amino)-3-methoxy-5-methylheptanoyl)pyrrolidin-2-yl)-3-methoxy-2-methylpropanamido)-3-phenylpropanoate

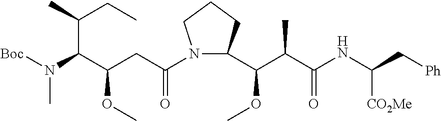 [SMILES: CC[C@H](C)[C@@H]([C@@H](CC(=O)N1CCC[C@H]1[C@H](OC)[C@@H](C)C(=O)N[C@@H](Cc1ccccc1)C(=O)OC)OC)N(C)C(=O)OC(C)(C)C]

To a solution of the Boc-deprotected product of (S)-tert-butyl 2-((1R,2R)-1-methoxy-3-(((S)-1-methoxy-1-oxo-3-phenylpropan-2-yl)amino)-2-methyl-3-oxopropyl)pyrrolidine-1-carboxylate (0.29 mmol) and (3R,4S,5S)-4-((tert-butoxycarbonyl)(methyl)amino)-3-methoxy-5-methylheptanoic acid (96.6 mg, 0.318 mmol) in DMF (5 mL) at 0° C. was added diethyl cyanophosphonate (58 μL, 0.347 mmol), followed by Et₃N (109 μL, 0.78 mmol). The reaction mixture was stirred at 0° C. for 2 h, then warmed to r.t. and stirred overnight. The reaction mixture was diluted with ethyl acetate (80 mL), washed with 1 N aqueous potassium hydrogen sulfate (40 mL), water (40 mL), saturated aqueous sodium hydrogen carbonate (40 mL), and saturated aqueous sodium chloride (40 mL), dried over Na₂SO₄ and concentrated in vacuo. The residue was purified by column chromatography (15-75% ethyl acetate/hexanes) to afford the title compound (150 mg, 81% yield) as a white solid. LC-MS (ESI) m/z calcd. for C₃₄H₅₅N₃O₈ [M+H]⁺: 634.40, found: 634.40.

### Example 102. Synthesis of (S)-methyl 2-((2R,3R)-3-((S)-1-((3R,4S,5S)-4-((S)-2-((tert-butoxycarbonyl)amino)-N,3-dimethylbutanamido)-3-methoxy-5-methylheptanoyl)pyrrolidin-2-yl)-3-methoxy-2-methylpropanamido)-3-phenylpropanoate

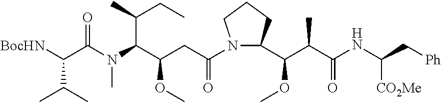 [SMILES: CC[C@H](C)[C@@H]([C@@H](CC(=O)N1CCC[C@H]1[C@H](OC)[C@@H](C)C(=O)N[C@@H](Cc1ccccc1)C(=O)OC)OC)N(C)C(=O)[C@@H](NC(=O)OC(C)(C)C)C(C)C]

To a solution of the Boc-deprotected product of (S)-methyl 2-((2R,3R)-3-((S)-1-((3R,4S,5S)-4-((tert-butoxycarbonyl)(methyl)amino)-3-methoxy-5-methylheptanoyl)-pyrrolidin-2-yl)-3-methoxy-2-methylpropanamido)-3-phenylpropanoate (0.118 mmol) and Boc-Val-OH (51.8 mg, 0.236 mmol) in DCM (5 mL) at 0° C. was added BroP (70.1 mg, 0.184 mmol), followed by diisopropylethylamine (70 μL, 0.425 mmol). The mixture was shielded from light and stirred at 0° C. for 30 min then at r.t. for 2 days. The reaction mixture was diluted with ethyl acetate (80 mL), washed with 1 N aqueous potassium hydrogen sulfate (40 mL), water (40 mL), saturated aqueous sodium hydrogen carbonate (40 mL), and saturated aqueous sodium chloride (40 mL), dried over Na₂SO₄ and concentrated in vacuo. The residue was purified by column chromatography (20-100% ethyl acetate/hexanes) to afford the title compound (67 mg, 77% yield) as a white solid. LC-MS (ESI) m/z calcd. for C₃₉H6₄N₄O₉ [M+H]+: 733.47, found: 733.46.

### Example 103. Synthesis of (S)-methyl 2-((2R,3R)-3-((S)-1-((6S,9S,12S,13R)-12-((S)-sec-butyl)-6,9-diisopropyl-13-methoxy-2,2,5,11-tetramethyl-4,7,10-trioxo-3-oxa-5,8,11-triazapentadecan-15-oyl)pyrrolidin-2-yl)-3-methoxy-2-methylpropanamido)-3-phenylpropanoate

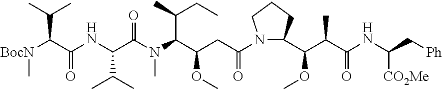 [SMILES: CC[C@H](C)[C@@H]([C@@H](CC(=O)N1CCC[C@H]1[C@H](OC)[C@@H](C)C(=O)N[C@@H](Cc1ccccc1)C(=O)OC)OC)N(C)C(=O)[C@@H](NC(=O)[C@H](C(C)C)N(C)C(=O)OC(C)(C)C)C(C)C]

To a solution of the Boc-deprotected product of (S)-methyl 2-((2R,3R)-3-((S)-1-((3R,4S,5S)-4-((S)-2-((tert-butoxycarbonyl)amino)-N,3-dimethylbutanamido)-3-methoxy-5-methylheptanoyl)pyrrolidin-2-yl)-3-methoxy-2-methylpropanamido)-3-phenylpropanoate (0.091 mmol) and Boc-N-Me-Val-OH (127 mg, 0.548 mmol) in DMF (5 mL) at 0° C. was added diethyl cyanophosphonate (18.2 μL, 0.114 mmol), followed by N-methylmorpholine (59 μL, 0.548 mmol). The reaction mixture was stirred at 0° C. for 2 h, then warmed to r.t. and stirred overnight. The reaction mixture was diluted with ethyl acetate (80 mL), washed with 1 N aqueous potassium hydrogen sulfate (40 mL), water (40 mL), saturated aqueous sodium hydrogen carbonate (40 mL), and saturated aqueous sodium chloride (40 mL), dried over sodium sulfate, and concentrated in vacuo. The residue was purified by column chromatography (20-100% ethyl acetate/hexanes) to afford the title compound (30 mg, 39% yield) as a white solid. LC-MS (ESI) m/z calcd. for C₄₅H₇₅N₅O₁₀ [M+H]+: 846.55. found: 846.56.

### Example 104. Synthesis of (S)-methyl 2-((2R,3R)-3-((S)-1-((3R,4S,5S)-4-((S)—N,3-dimethyl-2-((S)-3-methyl-2-(methylamino)butanamido)butanamido)-3-methoxy-5-methyl-heptanoyl)pyrrolidin-2-yl)-3-methoxy-2-methylpropanamido)-3-phenylpropanoate

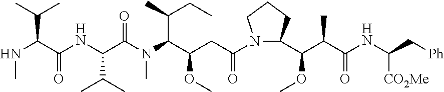 [SMILES: CC[C@H](C)[C@@H]([C@@H](CC(=O)N1CCC[C@H]1[C@H](OC)[C@@H](C)C(=O)N[C@@H](Cc1ccccc1)C(=O)OC)OC)N(C)C(=O)[C@@H](NC(=O)[C@@H](NC)C(C)C)C(C)C]

To a solution of (S)-methyl 2-((2R,3R)-3-((S)-1-((6S,9S,12S,13R)-12-((S)-sec-butyl)-6,9-diisopropyl-13-methoxy-2,2,5,11-tetramethyl-4,7,10-trioxo-3-oxa-5,8,11-triazapentadecan-15-oyl)pyrrolidin-2-yl)-3-methoxy-2-methylpropanamido)-3-phenylpropanoate (75.0 mg, 0.0886 mmol) in methylene chloride (5 mL) was added trifluoroacetic acid (2 mL) at room temperature. After being stirred at room temperature for 1 h, the reaction mixture was concentrated in vacuo. Co-evaporation with toluene gave the deprotected title product, which was used without further purification.

### Example 105. Synthesis of (S)-2-((2R,3R)-3-((S)-1-((3R,4S,5S)-4-((S)—N,3-dimethyl-2-((S)-3-methyl-2-(methylamino)butanamido)butanamido)-3-methoxy-5-methylheptanoyl)-pyrrolidin-2-yl)-3-methoxy-2-methylpropanamido)-3-phenylpropanoic Acid

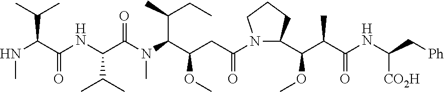 [SMILES: CC[C@H](C)[C@@H]([C@@H](CC(=O)N1CCC[C@H]1[C@H](OC)[C@@H](C)C(=O)N[C@@H](Cc1ccccc1)C(=O)O)OC)N(C)C(=O)[C@@H](NC(=O)[C@@H](NC)C(C)C)C(C)C]

(S)-Methyl 2-((2R,3R)-3-((S)-1-((3R,4S,5S)-4-((S)—N,3-dimethyl-2-((S)-3-methyl-2-(methylamino)butanamido)butanamido)-3-methoxy-5-methyl-heptanoyl)pyrrolidin-2-yl)-3-methoxy-2-methylpropanamido)-3-phenylpropanoate (25 mg, 0.030 mmol) in the mixture of conc. HCl (0.3 ml) and 1,4-dioxane (0.9 ml) was stirred at r.t. for 35 min. The mixture was diluted with EtOH (1.0 ml) and toluene (1.0 ml), concentrated and co-evaporated with EtOH/toluene (2:1) to afford the title compound as a white solid (22 mg, ˜100% yield), which was used in the next step without further purification. LC-MS (ESI) m/z calcd. for C₃₉H₆₆N₅O₈ [M+H]+: 732.48, found: 732.60.

### Example 106. Synthesis of (2S)-2-((2R,3R)-3-((2S)-1-((11S,14S,17S)-1-azido-17-((R)-sec-butyl)-11,14-diisopropyl-18-methoxy-10,16-dimethyl-9,12,15-trioxo-3,6-dioxa-10,13,16-triazai-cosan-20-oyl)pyrrolidin-2-yl)-3-methoxy-2-methylpropanamido)-3-phenylpropanoic Acid

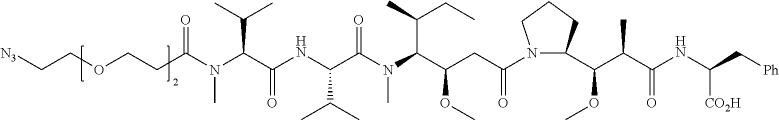

To the crude (S)-2-((2R,3R)-3-((S)-1-((3R,4S,5S)-4-((S)—N,3-dimethyl-2-((S)-3-methyl-2-(methylamino)butanamido)butanamido)-3-methoxy-5-methylheptanoyl)-pyrrolidin-2-yl)-3-methoxy-2-methylpropanamido)-3-phenylpropanoic acid (22 mg, 0.030 mmol) in a mixture of DMA (0.8 ml) and NaH₂PO₄ buffer solution (pH 7.5, 1.0 M, 0.7 ml) was added 2,5-dioxopyrrolidin-1-yl 3-(2-(2-azidoethoxy)ethoxy)propanoate (18.0 mg, 0.060 mmol) in four portions in 2 h. The mixture was stirred overnight, concentrated and purified on SiO₂ column chromatography (CH₃OH/CH₂Cl₂/HOAc 1:8:0.01) to afford the title compound (22.5 mg, 82% yield). LC-MS (ESI) m/z calcd. for C₄₆H₇₇N₈O₁₁ [M+H]+: 917.56, found: 917.60.

### Example 107. Synthesis of (2S)-2-((2R,3R)-3-((2S)-1-((11S,14S,17S)-1-amino-17-((R)-sec-butyl)-11,14-diisopropyl-18-methoxy-10,16-dimethyl-9,12,15-trioxo-3,6-dioxa-10,13,16-triazaicosan-20-oyl)pyrrolidin-2-yl)-3-methoxy-2-methylpropanamido)-3-phenylpropanoic Acid

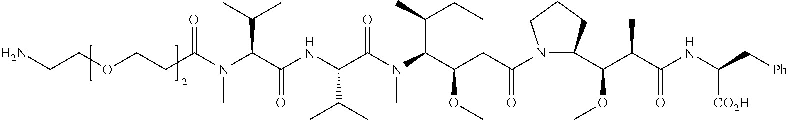

To (2S)-2-((2R,3R)-3-((2S)-1-((11S,14S,17S)-1-azido-17-((R)-sec-butyl)-11,14-diisopropyl-18-methoxy-10,16-dimethyl-9,12,15-trioxo-3,6-dioxa-10,13,16-triazai-cosan-20-oyl)pyrrolidin-2-yl)-3-methoxy-2-methylpropanamido)-3-phenylpropanoic acid (22.0 mg, 0.024 mmol) in methanol (5 ml) in a hydrogenation bottle was added Pd/C (5 mg, 10% Pd, 50% wet). After air was vacuumed out and 25 psi H₂ was conducted in, the mixture was shaken for 4 h, filtered through Celite. The filtrate was concentrated to afford the crude title product (˜20 mg, 92% yield), which was used in the next step without further purification. ESI MS m/z+ C₄₆H₇₉N₆O₁₁ (M+H), cacld. 891.57. found 891.60.

### Example 108. Synthesis of (S)-2-((2R,3R)-3-((S)-1-((6S,9S,12S,13R)-12-((S)-sec-butyl)-6,9-diisopropyl-13-methoxy-2,2,5,11-tetramethyl-4,7,10-trioxo-3-oxa-5,8,11-triazapenta-decan-15-oyl)pyrrolidin-2-yl)-3-methoxy-2-methylpropanamido)-3-phenylpropanoic Acid

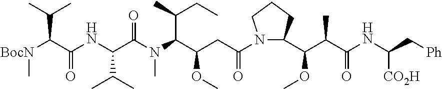 [SMILES: CC[C@H](C)[C@@H]([C@@H](CC(=O)N1CCC[C@H]1[C@H](OC)[C@@H](C)C(=O)N[C@@H](Cc1ccccc1)C(=O)O)OC)N(C)C(=O)[C@@H](NC(=O)[C@H](C(C)C)N(C)C(=O)OC(C)(C)C)C(C)C]

To a solution of (S)-methyl 2-((2R,3R)-3-((S)-1-((6S,9S,12S,13R)-12-((S)-sec-butyl)-6,9-diisopropyl-13-methoxy-2,2,5,11-tetramethyl-4,7,10-trioxo-3-oxa-5,8,11-triazapentadecan-15-oyl)pyrrolidin-2-yl)-3-methoxy-2-methylpropanamido)-3-phenylpropanoate (30 mg, 0.035 mmol) in THF (1.0 ml) was added LiOH in water (1.0M, 0.8 ml). The mixture was stirred at r.t. for 35 min, neutralized with 0.5 M H₃PO₄ to pH 6, concentrated and purified on SiO₂ column chromatography (CH₃OH/CH₂Cl₂/HOAc 1:10:0.01) to afford the title compound (25.0 mg, 85% yield). LC-MS (ESI) m/z calcd. for C₄₄H₇₄N₅O₁₀ [M+H]⁺: 832.54, found: 832.60.

### Example 109. Synthesis of (S)-2-((2R,3R)-3-((S)-1-((3R,4S,5S)-4-((S)—N,3-dimethyl-2-((S)-3-methyl-2-(methylamino)butanamido)butanamido)-3-methoxy-5-methylheptanoyl)-pyrrolidin-2-yl)-3-methoxy-2-methylpropanamido)-3-phenylpropanoic Acid

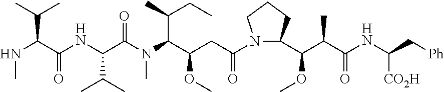 [SMILES: CC[C@H](C)[C@@H]([C@@H](CC(=O)N1CCC[C@H]1[C@H](OC)[C@@H](C)C(=O)N[C@@H](Cc1ccccc1)C(=O)O)OC)N(C)C(=O)[C@@H](NC(=O)[C@@H](NC)C(C)C)C(C)C]

To a solution of (S)-2-((2R,3R)-3-((S)-1-((6S,9S,12S,13R)-12-((S)-sec-butyl)-6,9-diisopropyl-13-methoxy-2,2,5,11-tetramethyl-4,7,10-trioxo-3-oxa-5,8,11-triazapenta-decan-15-oyl)pyrrolidin-2-yl)-3-methoxy-2-methylpropanamido)-3-phenylpropanoic acid (25 mg, 0.030 mmol) in dioxane (2.0 ml) was added HCl (12.0M, 0.6 ml). The mixture was stirred at r.t. for 30 min, diluted with dioxane (4 ml) and toluene (4 ml), concentrated and purified on C-18 HPLC column chromatography eluted with MeOH and water (L200 mm×Φ20 mm, v=9 ml/min, from 5% methanol to 40% methanol in 40 min) to afford the title compound (20.0 mg, 90% yield). LC-MS (ESI) m/z calcd. for C₃₉H₆₆N₅O₈ [M+H]⁺: 732.48, found: 732.90.

### Example 110. Synthesis of (S)-methyl 2-((2R,3R)-3-((S)-1-((5S,8S,11S,14S,15R)-14-((S)-sec-butyl)-8,11-diisopropyl-15-methoxy-5,7,13-trimethyl-3,6,9,12-tetraoxo-1-phenyl-2-oxa-4,7,10,13-tetraazaheptadecan-17-oyl)pyrrolidin-2-yl)-3-methoxy-2-methylpropanamido)-3-phenylpropanoate

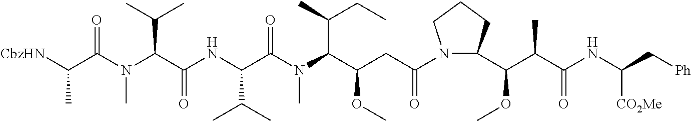 [SMILES: CC[C@H](C)[C@@H]([C@@H](CC(=O)N1CCC[C@H]1[C@H](OC)[C@@H](C)C(=O)N[C@@H](Cc1ccccc1)C(=O)OC)OC)N(C)C(=O)[C@@H](NC(=O)[C@H](C(C)C)N(C)C(=O)[C@H](C)NC(=O)OCc1ccccc1)C(C)C]

To a solution of MMAF-OMe (0.132 g, 0.178 mmol, 1.0 eq.) and Z-L-Alanine (0.119 g, 0.533 mmol, 3.0 eq.) in anhydrous DCM (10 mL) at 0° C. was added HATU (0.135 g, 0.356 mmol, 2.0 eq.) and NMM (0.12 mL, 1.07 mmol, 6.0 eq.) in sequence. The reaction was stirred at 0° C. for 10 minutes, then warmed to room temperature and stirred overnight. The mixture was diluted with DCM and washed with water and brine, dried over anhydrous Na₂SO₄, concentrated and purified by SiO₂ column chromatography (20:1 DCM/MeOH) to give the title compound as a white foamy solid (0.148 g, 88% yield). ESI MS m/z: calcd for C₅₁H₇₉N₆O₁₁ [M+H]⁺ 951.6. found 951.6.

### Example 111. Synthesis of (S)-methyl 2-((2R,3R)-3-((S)-1-((3R,4S,5S)-4-((S)-2-((S)-2-((S)-2-amino-N-methylpropanamido)-3-methylbutanamido)-N,3-dimethylbutanamido)-3-methoxy-5-methylheptanoyl)pyrrolidin-2-yl)-3-methoxy-2-methylpropanamido)-3-phenylpropanoate

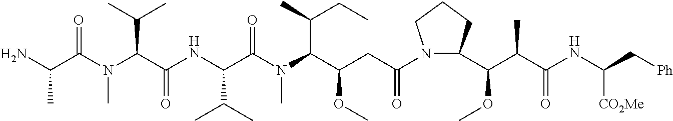 [SMILES: CC[C@H](C)[C@@H]([C@@H](CC(=O)N1CCC[C@H]1[C@H](OC)[C@@H](C)C(=O)N[C@@H](Cc1ccccc1)C(=O)OC)OC)N(C)C(=O)[C@@H](NC(=O)[C@H](C(C)C)N(C)C(=O)[C@H](C)N)C(C)C]

To a solution of (S)-methyl 2-((2R,3R)-3-((S)-1-((5S,8S,11S,14S,15R)-14-((S)-sec-butyl)-8,11-diisopropyl-15-methoxy-5,7,13-trimethyl-3,6,9,12-tetraoxo-1-phenyl-2-oxa-4,7,10,13-tetraazaheptadecan-17-oyl)pyrrolidin-2-yl)-3-methoxy-2-methylpropanamido)-3-phenylpropanoate (0.148 g, 0.156 mmol, 1.0 equiv) in MeOH (5 mL) was added Pd/C (0.100 g, 10% Pd/C, 50% wet) in a hydrogenation bottle. The mixture was shaken for 5 h then filtered through a Celite pad. The filtrate was concentrated to give the title compound as a white foamy solid (0.122 g, 96% yield). ESI MS m/z: calcd for C₄₃H₇₃N₆O₉ [M+H]⁺ 817.5. found 817.5.

### Example 112. Synthesis of (S)-2-((2R,3R)-3-((S)-1-((8S,11S,14S,17S,20S,21R)-20-((S)-sec-butyl)-14,17-diisopropyl-21-methoxy-8,11,13,19-tetramethyl-3,6,9,12,15,18-hexaoxo-5-propiolamido-4,7,10,13,16,19-hexaazatricos-1-yn-23-oyl)pyrrolidin-2-yl)-3-methoxy-2-methylpropanamido)-3-phenylpropanoic Acid (A-4)

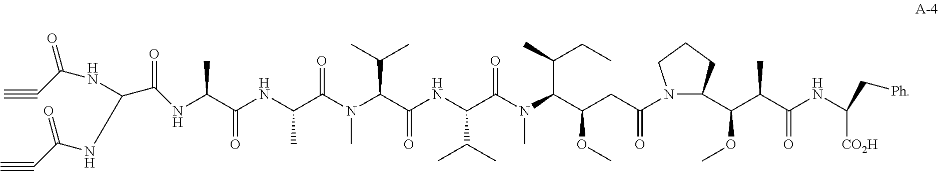 [SMILES: C#CC(=O)NC(NC(=O)C#C)C(=O)N[C@@H](C)C(=O)N[C@@H](C)C(=O)N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@@H](Cc1ccccc1)C(=O)O)OC)C(C)C)C(C)C]

To Compound S)-methyl 2-((2R,3R)-3-((S)-1-((3R,4S,5S)-4-((S)-2-((S)-2-((S)-2-amino-N-methylpropanamido)-3-methylbutanamido)-N,3-dimethylbutanamido)-3-methoxy-5-methyl-heptanoyl)pyrrolidin-2-yl)-3-methoxy-2-methylpropanamido)-3-phenylpropanoate (20 mg, 0.027 mmol) in the mixture of DMA (2 ml) and 0.1 M Na₂HPO₄, pH 8.0 (1 ml) was added (S)-2,5-dioxopyrrolidin-1-yl 2-((S)-2-(2,2-dipropiolamido-acetamido)propanamido)propanoate (20.1 mg, 0.046 mmol) in three portions in 3 h and the mixture was then stirred for another 12 hr. The mixture was concentrated, and purified by reverse phase HPLC (200 (L) mm×10(d) mm, C₁₈ column, 10-100% acetonitrile/water in 40 min, v=8 ml/min) to afford the title compound (22.1 mg, 78% yield). ESI MS m/z: calcd for C₅₃H₈₀N₉O₁₃ [M+H]⁺ 1050.58. found 1050.96.

### Example 113. Synthesis of (Z)-4-hydrazinyl-4-oxobut-2-enoic Acid, Hydrochloride Salt

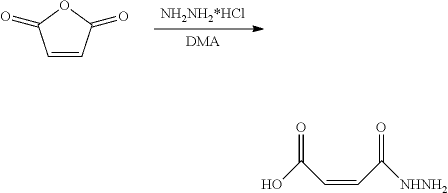

Hydrazine hydrochloride (7.00 g, 102.1 mmol) in DMA (100 ml) was added maleic anhydride (10.01 g). The mixture was stirred overnight, concentrated and recrystallized in EtOH to form the title compound (12.22 g, 92% yield). ESI MS m/z: calcd for C₄H₇N₂O₃ [M+H]⁺ 131.04. found 131.20.

### Example 114. Synthesis of (2S)-2-((2R,3R)-3-((2S)-1-((11S,14S,17S,18R)-17-((S)-sec-butyl)-11,14-diisopropyl-18-methoxy-10,16-dimethyl-9,12,15-trioxo-1-((bis(2-(Z)-3-carboxyacrylhydrazinyl)phosphoryl)amino)-3,6-dioxa-10,13,16-triazaicosan-20-oyl)pyrrolidin-2-yl)-3-methoxy-2-methylpropanamido)-3-phenylpropanoic Acid (A-5)

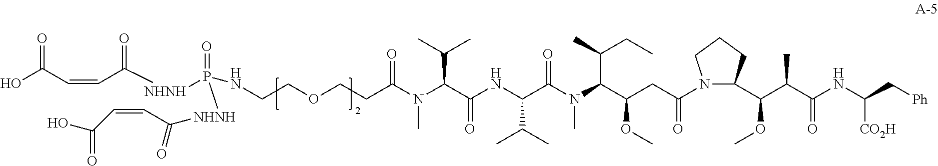

To compound (Z)-4-hydrazinyl-4-oxobut-2-enoic acid HCl salt (22.0 mg, 0.132 mmol) in the mixture of THF (5 ml) and DIPEA (10 μl, 0.057 mmol) at 0° C. was added POCl₃ (10.1 mg, 0.0665 mmol). After stirred at 0° C. for 20 min, the mixture was warmed to room temperature and kept to stirring for another 4 h. Then to the mixture was added (S)-2-((2R,3R)-3-((S)-1-((11S,14S,17S,18R)-1-amino-17-((S)-sec-butyl)-11,14-diisopropyl-18-methoxy-10,16-dimethyl-9,12,15-trioxo-3,6-dioxa-10,13,16-triazaicosan-20-oyl)pyrrolidin-2-yl)-3-methoxy-2-methylpropanamido)-3-phenylpropanoic acid (60 mg, 0.067 mmol) and DIPEA (20 μl, 0.114 mmol). The mixture was stirred at 50° C. for overnight, concentrated, and purified by reverse phase HPLC (200 (L) mm×10(d) mm, C₁₈ column, 10-100% acetonitrile/water in 40 min, v=8 ml/min) to afford the title compound (25.6 mg, 31% yield). ESI MS m/z: calcd for C₅₄H₈₄N₈₈O₁₈P [M+H]⁺ 1195.59. found 1196.10.

### Example 115. Synthesis of (S, E)-2-methyl-N-(3-methylbutan-2-ylidene)propane-2-sulfonamide

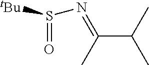

To a solution of (S)-2-methylpropane-2-sulfinamide (100 g, 0.825 mol, 1.0 eq.) in 1 L THF was added Ti(OEt)₄ (345 mL, 1.82 mol, 2.2 eq.) and 3-methyl-2-butanone (81 mL, 0.825 mol, 1.0 eq.) under N₂ at r.t. The reaction mixture was refluxed for 16 h, then cooled to r.t. and poured onto iced water. The mixture was filtered and the filter cake was washed with EtOAc. The organic layer was separated, dried over anhydrous Na₂SO₄ and concentrated to give a residue which was purified by vacuum distillation (15-20 torr, 95° C.) to afforded the title product (141 g, 90% yield) as a yellow oil. 1H NMR (500 MHz, CDCl₃) δ 2.54-2.44 (m, 1H), 2.25 (s, 3H), 1.17 (s, 9H), 1.06 (dd, J=6.9, 5.1 Hz, 6H). MS ESI m/z calcd for C₉H₁₉NaNOS [M+Na]⁺212.12; found 212.11.

### Example 116. Synthesis of (2S,3S)-2-azido-3-methylpentanoic Acid

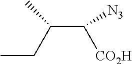

To a solution of NaN₃ (20.0 g, 308 mmol) in a mixture of water (50 mL) and dichloromethane (80 mL), cooled at 0° C., Tf₂O (10 mL, 59.2 mmol, 2.0 eq.) was added slowly. After addition, the reaction was stirred at 0° C. for 2 h, then the organic phase was separated and the aqueous phase was extracted with dichloromethane (2×40 mL). The combined organic phases were washed with saturated NaHCO₃ solution and used as is. The dichloromethane solution of triflyl azide was added to a mixture of (L)-isoleucine (4.04 g, 30.8 mmol, 1.0 eq.), K₂CO₃ (6.39 g, 46.2 mmol, 1.5 eq.), CuSO₄.5H₂O (77.4 mg, 0.3 mmol, 0.01 eq.) in water (100 ml) and methanol (200 ml). The mixture was stirred at r.t. for 16 h. The organic solvents were removed under reduced pressure and the aqueous phase was diluted with water (250 mL) and acidified to pH 6 with concentrated HCl and diluted with phosphate buffer (0.25 M, pH 6.2, 250 mL). The aqueous layer was washed with EtOAc (5×100 mL) to remove the sulfonamide by-product, and then acidified to pH 2 with concentrated HCl, extracted with EtOAc (3×150 mL). The combined organic layers were dried over anhydrous Na₂SO₄, filtered and concentrated to give the title product (4.90 g, 99% yield) as colorless oil. ¹H NMR (500 MHz, CDCl₃) δ 12.01 (s, 1H), 3.82 (d, J=5.9 Hz, 1H), 2.00 (ddd, J=10.6, 8.6, 5.5 Hz, 1H), 1.54 (dqd, J=14.8, 7.5, 4.4 Hz, 1H), 1.36-1.24 (m, 1H), 1.08-0.99 (m, 3H), 0.97-0.87 (m, 3H).

### Example 117. Synthesis of D-N-methyl Pipecolinic Acid

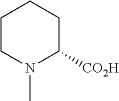

To a solution of D-pipecolinic acid (10.0 g, 77.4 mmol, 1.0 eq.) in methanol (100 mL) was added formaldehyde (37% aqueous solution, 30.8 mL, 154.8 mmol, 2.0 eq.), followed by Pd/C (10 wt %, 1.0 g). The reaction mixture was stirred under H₂ (1 atm) overnight, and then filtered through Celite, with washing of the filter pad with methanol. The filtrate was concentrated under reduced pressure to afford the title compound (10.0 g, 90% yield) as a white solid.

### Example 118. Synthesis of (R)-perfluorophenyl 1-methylpiperidine-2-carboxylate

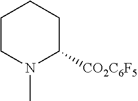

To a solution of D-N-methyl pipecolinic acid (2.65 g, 18.5 mmol) in EtOAc (50 mL) were added pentafluorophenol (3.75 g, 20.4 mmol) and DCC (4.21 g, 20.4 mmol). The reaction mixture was stirred at r.t. for 16 h, and then filtered over Celite. The filter pad was washed with 10 mL of EtOAc. The filtrate was used for the next step without further purification or concentration. MS ESI m/z calcd for C₁₃H₁₃F₅NO₂ [M+H]⁺ 309.08; found 309.60.

### Example 119. Synthesis of perfluorophenyl 2-(dimethylamino)-2-methylpropanoate

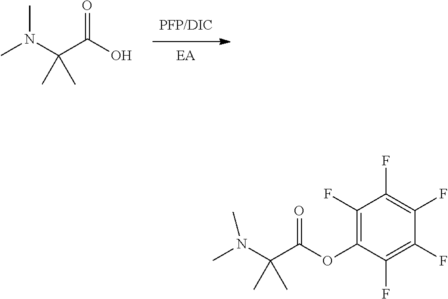

To a solution of 2-(dimethylamino)-2-methylpropanoic acid (5.00 g, 38.10 mmol) in ethyl acetate (200 ml) at 0° C. was added 2,3,4,5,6-pentafluorophenol (10.4 g, 57.0 mmol) followed by addition of DIC (8.8 mL, 57.0 mmol). The reaction mixture was warmed to RT, stirred overnight and filtered. The filtrate was concentrated to afford the title compound (12.0 g, >100% yield) which was used for the next step without further purification. MS ESI m/z calcd for C₁₂H₁₃F₅NO₂ [M+H]⁺ 298.08; found 298.60.

### Example 120. Synthesis of 2,2-diethoxyethanethioamide

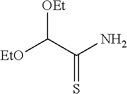

2,2-diethoxyacetonitrile (100 g, 0.774 mol, 1.0 eq.) was mixed with (NH₄)₂S aqueous solution (48%, 143 mL, 1.05 mol, 1.36 eq.) in methanol (1.5 L) at room temperature. After stirring for 16 h, the reaction mixture was concentrated and the residue was taken up in dichloromethane, washed with saturated NaHCO₃ solution and brine, dried over anhydrous Na₂SO₄ and concentrated. The residue was triturated with a solvent mixture of petroleum ether and dichloromethane. After filtration, the desired title product as a white solid was collected (100 g, 79% yield). H NMR (500 MHz, CDCl₃) δ 7.81 (d, J=71.1 Hz, 2H), 5.03 (s, 1H), 3.73 (dq, J=9.4, 7.1 Hz, 2H), 3.64 (dq, J=9.4, 7.0 Hz, 2H), 1.25 (t, J=7.1 Hz, 6H).

### Example 121. Synthesis of ethyl 2-(diethoxymethyl)thiazole-4-carboxylate

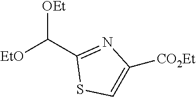 [SMILES: CCOC(=O)c1csc(C(OCC)OCC)n1]

90 g of molecular sieves (3A) was added to a mixture of 2,2-diethoxyethanethioamide (100 g, 0.61 mol, 1.0 eq.) and ethyl bromopyruvate (142 mL, 1.1 mol, 1.8 eq.) in 1 L EtOH. The mixture was refluxed (internal temperature about 60° C.) for 1 h, then ethanol was removed on rotovap and the residue was taken up in dichloromethane. The solid was filtered off and the filtrate was concentrated and purified by column chromatography (PE/EtOAc 5:1-3:1) to give the title (thiazole carboxylate) compound (130 g, 82% yield) as a yellow oil.

### Example 122. Synthesis of ethyl 2-formylthiazole-4-carboxylate

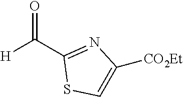 [SMILES: CCOC(=O)c1csc(C=O)n1]

To a solution of 2-(diethoxymethyl)thiazole-4-carboxylate (130 g, 0.50 mol) in acetone (1.3 L) was added 2 N HCl (85 mL, 0.165 mol, 0.33 eq.). The reaction mixture was refluxed (internal temperature about 60° C.), monitored by TLC analysis until starting material was completely consumed (about 1-2 h). Acetone was removed under reduced pressure and the residue was taken up in dichloromethane (1.3 L), washed with saturated NaHCO₃ solution, water and brine, and then dried over anhydrous Na₂SO₄. The solution was filtered and concentrated under reduced pressure. The crude product was purified by recrystallization from petreolum ether and diethyl ether to afford the title compound as a white solid (40 g, 43% yield). ¹H NMR (500 MHz, CDCl₃) δ 10.08-10.06 (m, 1H), 8.53-8.50 (m, 1H), 4.49 (q, J=7.1 Hz, 2H), 1.44 (t, J=7.1 Hz, 3H). MS ESI m/z calcd for C₇H₈NO₃S [M+H]⁺ 186.01; found 186.01.

### Example 123. Synthesis of ethyl 2-((R,E)-3-(((S)-tert-butylsulfinyl)imino)-1-hydroxy-4-methylpentyl)thiazole-4-carboxylate

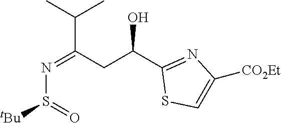 [SMILES: CCOC(=O)c1csc([C@H](O)C/C(=N\[S@@](=O)C(C)(C)C)C(C)C)n1]

To a solution of diisopropylamine (121 mL, 0.86 mol, 4.0 eq.) in dry THF (300 mL) was added n-butyllithium (2.5 M, 302 mL, 0.76 mol 3.5 eq.) at −78° C. under N₂. The reaction mixture was warmed to 0° C. over 30 min and then cooled back to −78°. (S, E)-2-methyl-N-(3-methylbutan-2-ylidene)propane-2-sulfonamide (57 g, 0.3 mol, 1.4 eq.) in THF (200 mL) was added. The reaction mixture was stirred for 1 h before ClTi(OⁱPr)₃ (168.5 g, 0.645 mol, 3.0 eq.) in THF (350 mL) was added dropwise. After stirring for 1 h, ethyl 2-formylthiazole-4-carboxylate (40 g, 0.215 mol, 1.0 eq.) dissolved in THF (175 mL) was added dropwise and the resulting reaction mixture was stirred for 2 h. The completion of the reaction was indicated by TLC analysis. The reaction was quenched by a mixture of acetic acid and THF (v/v 1:4, 200 mL), then poured onto iced water, extracted with EtOAc (4×500 mL). The organic phase was washed with water and brine, dried over anhydrous Na₂SO₄, filtered and concentrated. The residue was purified by column chromatography (DCM/EtOAc/PE 2:1:2) to afforded the title compound (60 g, 74% yield) as a colorless oil. ¹H NMR (500 MHz, CDCl₃) δ 8.13 (s, 1H), 6.63 (d, J=8.2 Hz, 1H), 5.20-5.11 (m, 1H), 4.43 (q, J=7.0 Hz, 2H), 3.42-3.28 (m, 2H), 2.89 (dt, J=13.1, 6.5 Hz, 1H), 1.42 (t, J=7.1 Hz, 3H), 1.33 (s, 9H), 1.25-1.22 (m, 6H). MS ESI m/z calcd for C₁₆H₂₆NaN₂O₄S₂ [M+Na]⁺397.13. found 397.11.

### Example 124. Synthesis of ethyl 2-((1R,3R)-3-((S)-1,1-dimethylethylsulfinamido)-1-hydroxy-4-methylpentyl)thiazole-4-carboxylate

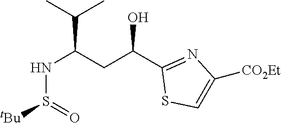 [SMILES: CCOC(=O)c1csc([C@H](O)C[C@@H](N[S@@](=O)C(C)(C)C)C(C)C)n1]

A solution of ethyl 2-((R,E)-3-(((S)-tert-butylsulfinyl)imino)-1-hydroxy-4-methylpentyl) thiazole-4-carboxylate (23.5 g, 62.7 mmol) dissolved in THF (200 mL) was cooled to −45° C. Ti(OEt)₄ (42.9 mL, 188 mmol, 3.0 eq.) was added slowly. After the completion of addition, the mixture was stirred for 1 h, before NaBH₄ (4.75 g, 126 mmol, 2.0 eq.) was added in portions. The reaction mixture was stirred at −45° C. for 3 h. TLC analysis showed some starting material still remained. The reaction was quenched with HOAc/THF (v/v 1:4, 25 mL), followed by EtOH (25 mL). The reaction mixture was poured onto ice (100 g) and warmed to r.t. After filtration over Celite, the organic phase was separated and washed with water and brine, dried over anhydrous Na₂SO₄, filtered, and concentrated. The residue was purified by column chromatography (EtOAc/PE 1:1) to deliver the title product (16.7 g, 71% yield) as a white solid. ¹H NMR (500 MHz, CDCl₃) δ 8.10 (s, 1H), 5.51 (d, J=5.8 Hz, 1H), 5.23-5.15 (m, 1H), 4.41 (q, J=7.0 Hz, 2H), 3.48-3.40 (m, 1H), 3.37 (d, J=8.3 Hz, 1H), 2.29 (t, J=13.0 Hz, 1H), 1.95-1.87 (m, 1H), 1.73-1.67 (m, 1H), 1.40 (t, J=7.1 Hz, 3H), 1.29 (s, 9H), 0.93 (d, J=7.3 Hz, 3H), 0.90 (d, J=7.2 Hz, 3H). MS ESI m/z calcd for C₁₆H₂₈NaN₂O₄S₂ [M+Na]⁺399.15. found 399.14.

### Example 125. Synthesis of ethyl 2-((1R,3R)-3-amino-1-hydroxy-4-methylpentyl)thiazole-4-carboxylate Hydrochloride

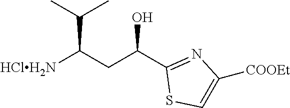

To a solution of ethyl 2-((1R,3R)-3-((S)-1,1-dimethylethylsulfinamido)-1-hydroxy-4-methylpentyl)thiazole-4-carboxylate (6.00 g, 16.0 mmol, 1.0 eq.) in ethanol (40 mL) was added 4 N HCl in dioxane (40 mL) slowly at 0° C. The reaction was allowed to warm to r.t. and stirred for 2.5 h then concentrated and triturated with petreolum ether. A white solid title compound (4.54 g, 92% yield) was collected and used in the next step.

### Example 126. Synthesis of ethyl 2-((1R,3R)-3-((2S,3S)-2-azido-3-methylpentanamido)-1-hydroxy-4-methylpentyl)thiazole-4-carboxylate

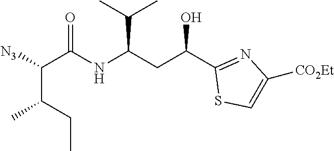 [SMILES: CCOC(=O)c1csc([C@H](O)C[C@@H](NC(=O)[C@@H](N=[N+]=[N-])[C@@H](C)CC)C(C)C)n1]

(2S,3S)-2-azido-3-methylpentanoic (5.03 g, 28.8 mmol, 2.0 eq.) was dissolved in THF (120 mL) and cooled to 0° C., to which NMM (6.2 mL, 56.0 mmol, 4.0 eq.) and isobutylchloroformate (3.7 mL, 28.8 mmol, 2.0 eq.) were added in sequence. The reaction was stirred at 0° C. for 30 min and r.t. 1.0 h, and then cooled back to 0° C. Ethyl 2-((1R,3R)-3-amino-1-hydroxy-4-methylpentyl)thiazole-4-carboxylate hydrochloride (4.54 g, 14.7 mmol, 1.0 eq.) was added in portions. After stirring at 0° C. for 30 min, the reaction was warmed to r.t. and stirred for 2 h. Water was added at 0° C. to quenched the reaction and the resulting mixture was extracted with ethyl acetate for three times. The combined organic layers were washed with 1N HCl, saturated NaHCO₃ and brine, dried over anhydrous Na₂SO₄, filtered and concentrated. The residue was purified by column chromatography (0-30% EtOAc/PE) to give a white solid title compound (4.55 g, 74% yield).

### Example 127. Synthesis of ethyl 2-((1R,3R)-3-((2S,3S)-2-azido-3-methylpentanamido)-4-methyl-1-((triethylsilyl)oxy)pentyl)thiazole-4-carboxylate

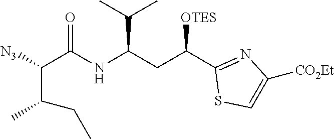

To a solution of ethyl 2-((1R,3R)-3-((2S,3S)-2-azido-3-methylpentanamido)-1-hydroxy-4-methylpentyl)thiazole-4-carboxylate (5.30 g, 12.8 mmol, 1.0 eq.) in CH₂Cl₂ (50 mL) was added imidazole (1.75 g, 25.6 mmol, 2.0 eq.), followed by chlorotriethylsilane (4.3 mL, 25.6 mmol, 2.0 eq.) at 0° C. The reaction mixture was allowed to warm to r.t. over 1 hour and stirred for an additional hour. Brine was added to the reaction mixture, the organic layer was separated and the aqueous layer was extracted with EtOAc. The combined organic phases were dried, filtered, concentrated under reduced pressure, and purified by column chromatography with a gradient of 15-35% EtOAc in petreolum ether to afford the title product (6.70 g, 99% yield) as a white solid. ¹H NMR (500 MHz, CDCl₃) δ 8.12 (s, 1H), 6.75 (d, J=8.0 Hz, 1H), 5.20-5.12 (m, 1H), 4.44 (q, J=7.0 Hz, 2H), 4.06-3.97 (m, 1H), 3.87 (d, J=3.8 Hz, 1H), 2.14 (d, J=3.8 Hz, 1H), 2.01-1.91 (m, 3H), 1.42 (t, J=7.1 Hz, 3H), 1.34-1.25 (m, 2H), 1.06 (d, J=6.8 Hz, 3H), 1.00-0.93 (m, 18H), 0.88 (dd, J=19.1, 6.8 Hz, 6H). MS ESI m/z calcd for C₂₄H₄₄N₅O₄SSi [M+H]⁺ 526.28. found 526.28.

### Example 128. Synthesis of ethyl 2-((1R,3R)-3-((2S,3S)-2-azido-N,3-dimethyl pentanamido)-4-methyl-1-((triethylsilyl)oxy)pentyl)thiazole-4-carboxylate

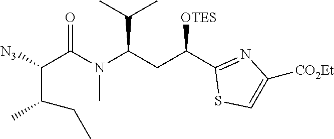

A solution of ethyl 2-((1R,3R)-3-((2S,3S)-2-azido-3-methylpentanamido)-4-methyl-1-((triethylsilyl)oxy)pentyl)thiazole-4-carboxylate (5.20 g, 9.9 mmol, 1.0 eq.) in THF (50 mL) was cooled to −45° C. and KHMDS (1M in toluene, 23.8 mL, 23.8 mmol, 2.4 eq.) was added. The resulting mixture was stirred at −45° C. for 20 min, followed by addition of methyl iodide (1.85 mL, 29.7 mmol, 3.0 eq.). The reaction mixture was warmed to r.t. over 4.5 h, then the reaction was quenched with EtOH (10 mL). The crude product was diluted with EtOAc (250 mL) and washed with brine (100 mL). The aqueous layer was extracted with EtOAc (3×50 ml). The organic layers were dried, filtered, concentrated and purified on column chromatography with a gradient of 15-35% EtOAc in petreolum ether to afford the title product (3.33 g, 63% yield) as a light yellow oil. ¹H NMR (500 MHz, CDCl₃) δ 8.09 (s, 1H), 4.95 (d, J=6.6 Hz, 1H), 4.41 (q, J=7.1 Hz, 2H), 3.56 (d, J=9.5 Hz, 1H), 2.98 (s, 3H), 2.27-2.06 (m, 4H), 1.83-1.70 (m, 2H), 1.41 (t, J=7.2 Hz, 3H), 1.29 (ddd, J=8.9, 6.8, 1.6 Hz, 3H), 1.01 (d, J=6.6 Hz, 3H), 0.96 (dt, J=8.0, 2.9 Hz, 15H), 0.92 (d, J=6.6 Hz, 3H), 0.90 (d, J=6.7 Hz, 3H). MS ESI m/z calcd for C₂₅H₄₆N₅O₄SSi [M+H]⁺ 540.30. found 540.30.

### Example 129. Synthesis of ethyl 2-((3S,6R,8R)-3-((S)-sec-butyl)-10,10-diethyl-6-isopropyl-5-methyl-1-((R)-1-methylpiperidin-2-yl)-1,4-dioxo-9-oxa-2,5-diaza-10-siladodecan-8-yl)thiazole-4-carboxylate

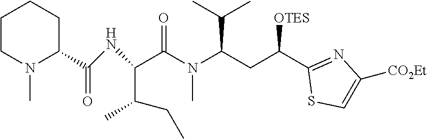

Dry Pd/C (10 wt %, 300 mg) and ethyl 2-((1R,3R)-3-((2S,3S)-2-azido-N,3-dimethyl pentanamido)-4-methyl-1-((triethylsilyl)oxy)pentyl)thiazole-4-carboxylate (3.33 g, 6.61 mmol) were added to (R)-perfluorophenyl 1-methylpiperidine-2-carboxylate in EtOAc. The reaction mixture was stirred under hydrogen atmosphere for 27 h, and then filtered through a plug of Celite, with washing of the filter pad with EtOAc. The combined organic portions were concentrated and purified by column chromatography with a gradient of 0-5% methanol in EtOAc to deliver the title product (3.90 g, 86% yield). MS ESI m/z calcd for C₃₂H₅₉N₄O₅SSi [M+H]⁺ 639.39. found 639.39.

### Example 130. Synthesis of ethyl 2-((1R,3R)-3-((2S,3S)—N,3-dimethyl-2-((R)-1-methyl piperidine-2-carboxamido)pentanamido)-1-hydroxy-4-methylpentyl)thiazole-4-carboxylate

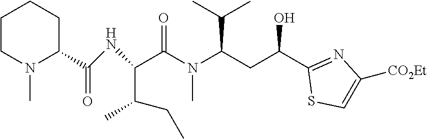 [SMILES: CCOC(=O)c1csc([C@H](O)C[C@H](C(C)C)N(C)C(=O)[C@@H](NC(=O)[C@H]2CCCCN2C)[C@@H](C)CC)n1]

Ethyl 2-((3S,6R,8R)-3-((S)-sec-butyl)-10,10-diethyl-6-isopropyl-5-methyl-1-((R)-1-methylpiperidin-2-yl)-1,4-dioxo-9-oxa-2,5-diaza-10-siladodecan-8-yl)thiazole-4-carboxylate (3.90 g, 6.1 mmol) was dissolved in deoxygenated AcOH/water/THF (v/v/v 3:1:1, 100 mL), and stirred at r.t. for 48 h. The reaction was then concentrated and purified on SiO₂ column chromatography (2:98 to 15:85 MeOH/EtOAc) to afford the title compound (2.50 g, 72% yield over 2 steps). MS ESI m/z calcd for C₂₆H₄₅N₄O₅S [M+H]⁺ 525.30. found 525.33.

### Example 131. Synthesis of 2-((1R,3R)-3-((2S,3S)—N,3-dimethyl-2-((R)-1-methylpiperidine-2-carboxamido)pentanamido)-1-hydroxy-4-methylpentyl)thiazole-4-carboxylic Acid

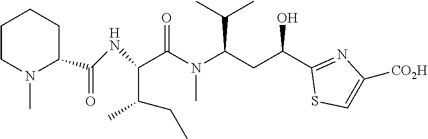 [SMILES: CC[C@H](C)[C@H](NC(=O)[C@H]1CCCCN1C)C(=O)N(C)[C@H](C[C@@H](O)c1nc(C(=O)O)cs1)C(C)C]

An aqueous solution of LiOH (0.4 N, 47.7 mL, 19.1 mmol, 4.0 eq.) was added to a solution of ethyl 2-((1R,3R)-3-((2S,3S)—N,3-dimethyl-2-((R)-1-methyl piperidine-2-carboxamido)-pentanamido)-1-hydroxy-4-methylpentyl)thiazole-4-carboxylate (2.50 g, 4.76 mmol, 1.0 eq.) in dioxane (47.7 mL) at 0° C. The reaction mixture was stirred at r.t. for 2 h and then concentrated. SiO₂ column chromatographic purification (100% CH₂Cl₂ then CH₂Cl₂/MeOH/NH₄OH 80:20:1) afforded the title compound (2.36 g, 99% yield) as an amorphous solid. MS ESI m/z calcd for C₂₄H₄₁N₄O₅S [M+H]⁺ 497.27. found 497.28.

### Example 132. Synthesis of 2-((1R,3R)-1-acetoxy-3-((2S,3S)—N,3-dimethyl-2-((R)-1-methylpiperidine-2-carboxamido)pentanamido)-4-methylpentyl)thiazole-4-carboxylic Acid

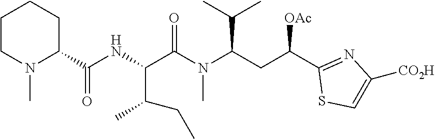 [SMILES: CC[C@H](C)[C@H](NC(=O)[C@H]1CCCCN1C)C(=O)N(C)[C@H](C[C@@H](OC(C)=O)c1nc(C(=O)O)cs1)C(C)C]

To a solution of 2-((1R,3R)-3-((2S,3S)—N,3-dimethyl-2-((R)-1-methylpiperidine-2-carboxamido)pentanamido)-1-hydroxy-4-methylpentyl)thiazole-4-carboxylic acid (2.36 g, 4.75 mmol) in pyridine (50 mL) at 0° C., acetic anhydride (2.25 mL, 24 mmol) was added slowly. The reaction mixture was warmed to r.t. over 2 h and stirred at r.t. for 24 h. The reaction was concentrated and the residue was purified on reverse phase HPLC (C₁₈ column, 50 mm (d)×250 (mm), 50 ml/min, 10-90% acetonitrile/water in 45 min) to afford the title compound (2.25 g, 88% yield) as an amorphous white solid. MS ESI m/z calcd for C₂₆H₄₃N₄O₆S [M+H]⁺539.28. found 539.28.

### Example 133. Synthesis of (1R,3R)-3-((2S,3S)—N,3-dimethyl-2-((R)-1-methylpiperidine-2-carboxamido)pentanamido)-4-methyl-1-(4-(perfluorobenzoyl)thiazol-2-yl)pentyl Acetate

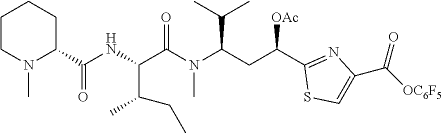

To a solution of 2-((1R,3R)-1-acetoxy-3-((2S,3S)—N,3-dimethyl-2-((R)-1-methyl-piperidine-2-carboxamido)pentanamido)-4-methylpentyl)thiazole-4-carboxylic acid (860 mg, 1.60 mmol, 1.0 eq.) in dichloromethane (20 mL) was added pentafluorophenol (440 mg, 2.40 mmol, 1.5 eq.) and N,N′-diisopropylcarbodiimide (220 mg, 1.75 mmol, 1.1 eq.) at 0° C. The reaction mixture was warmed to room temperature and stirred overnight. After the solvent was removed under reduced pressure, the reaction mixture was diluted with EtOAc (20 mL) then filtered over Celite. The filtrate was concentrated and purified on SiO₂ column chromatography (1:10 to 1:3 EtOAc/DCM) to afford the title compound (935.3 mg, 82% yield), which was used directly for the next step. MS ESI m/z calcd for C₃₂H₄₂F₅N₄O₆S [M+H]⁺ 704.28. found 704.60.

### Example 134. Synthesis of ethyl 2-((6S,9R,11R)-6-((S)-sec-butyl)-13,13-diethyl-9-isopropyl-2,3,3,8-tetramethyl-4,7-dioxo-12-oxa-2,5,8-triaza-13-silapentadecan-11-yl)thiazole-4-carboxylate

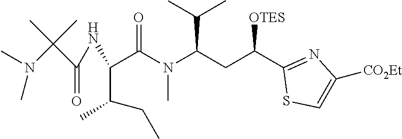

Dry Pd/C (10 wt %, 300 mg) and ethyl 2-((1R,3R)-3-((2S,3S)-2-azido-N,3-dimethyl pentanamido)-4-methyl-1-((triethylsilyl)oxy)pentyl)thiazole-4-carboxylate (3.33 g, 6.16 mmol) were added to perfluorophenyl 2-(dimethylamino)-2-methylpropanoate (˜2.75 g, 1.5 eq crude) in EtOAc. The reaction mixture was stirred under hydrogen atmosphere for 27 h, and then filtered through a plug of Celite, with washing of the filter pad with EtOAc. The combined organic portions were concentrated and purified by column chromatography with a gradient of 0-5% methanol in EtOAc to deliver the title product (3.24 g, 84% yield). MS ESI m/z calcd for C₃₁H₅₉N₄O₅SSi [M+H]⁺ 626.39. found 626.95.

### Example 135. Synthesis of ethyl 2-((1R,3R)-3-((2S,3S)-2-(2-(dimethylamino)-2-methylpropanamido)-N,3-dimethylpentanamido)-1-hydroxy-4-methylpentyl)thiazole-4-carboxylate

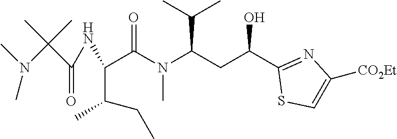 [SMILES: CCOC(=O)c1csc([C@H](O)C[C@H](C(C)C)N(C)C(=O)[C@@H](NC(=O)C(C)(C)N(C)C)[C@@H](C)CC)n1]

Ethyl 2-((6S,9R,11R)-6-((S)-sec-butyl)-13,13-diethyl-9-isopropyl-2,3,3,8-tetramethyl-4,7-dioxo-12-oxa-2,5,8-triaza-13-silapentadecan-11-yl)thiazole-4-carboxylate (3.20 g, 5.11 mmol) was dissolved in deoxygenated AcOH/water/THF (v/v/v 3:1:1, 100 mL), and stirred at r.t. for 48 h. The reaction was then concentrated and purified on SiO₂ column chromatography (2:98 to 15:85 MeOH/EtOAc) to afford the title compound (2.33 g, 89% yield). MS ESI m/z calcd for C₂₅H₄₅N₄O₅S [M+H]⁺ 512.30. found 512.45.

### Example 136. Synthesis of 2-((1R,3R)-3-((2S,3S)-2-(2-(dimethylamino)-2-methylpropanamido)-N,3-dimethylpentanamido)-1-hydroxy-4-methylpentyl)thiazole-4-carboxylic Acid

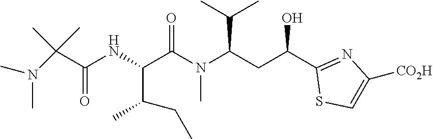 [SMILES: CC[C@H](C)[C@H](NC(=O)C(C)(C)N(C)C)C(=O)N(C)[C@H](C[C@@H](O)c1nc(C(=O)O)cs1)C(C)C]

An aqueous solution of LiOH (0.4 N, 47.7 mL, 19.1 mmol, 4.0 eq.) was added to a solution of ethyl 2-((1R,3R)-3-((2S,3S)-2-(2-(dimethylamino)-2-methylpropanamido)-N,3-dimethylpentanamido)-1-hydroxy-4-methylpentyl)thiazole-4-carboxylate (2.30 g, 4.50 mmol, 1.0 eq.) in dioxane (50 mL) at 0° C. The reaction mixture was stirred at r.t. for 2 h and then concentrated. SiO₂ column chromatographic purification (100% CH₂Cl₂ then CH₂Cl₂/MeOH/NH₄OH 80:20:1) afforded the title compound (2.13 g, 98% yield) as an amorphous solid. MS ESI m/z calcd for C₂₃H₄₁N₄O₅S [M+H]⁺ 485.27. found 485.55.

### Example 137. Synthesis of 2-((6S,9R,11R)-6-((S)-sec-butyl)-9-isopropyl-2,3,3,8-tetramethyl-4,7,13-trioxo-12-oxa-2,5,8-triazatetradecan-11-yl)thiazole-4-carboxylic Acid

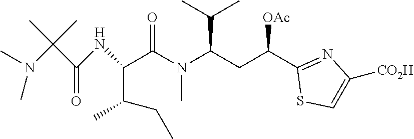 [SMILES: CC[C@H](C)[C@H](NC(=O)C(C)(C)N(C)C)C(=O)N(C)[C@H](C[C@@H](OC(C)=O)c1nc(C(=O)O)cs1)C(C)C]

To a solution of 2-((1R,3R)-3-((2S,3S)-2-(2-(dimethylamino)-2-methylpropanamido)-N,3-dimethylpentanamido)-1-hydroxy-4-methylpentyl)thiazole-4-carboxylic acid (2.10 g, 4.33 mmol) in pyridine (50 mL) at 0° C., acetic anhydride (2.25 mL, 24 mmol) was added slowly. The reaction mixture was warmed to r.t. over 2 h and stirred at r.t. for 24 h. The reaction was concentrated and the residue was purified on reverse phase HPLC (C₁₈ column, 50 mm (d)×250 (mm), 50 ml/min, 10-90% acetonitrile/water in 45 min) to afford the title compound (1.95 g, 86% yield) as an amorphous white solid. MS ESI m/z calcd for C₂₅H₄₃N₄O₆S [M+H]⁺526.28. found 526.80.

### Example 138. Synthesis of perfluorophenyl 2-((6S,9R,11R)-6-((S)-sec-butyl)-9-isopropyl-2,3,3,8-tetramethyl-4,7,13-trioxo-12-oxa-2,5,8-triazatetradecan-11-yl)thiazole-4-carboxylate

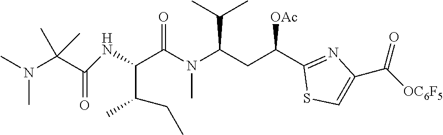

To a solution of 2-((6S,9R,11R)-6-((S)-sec-butyl)-9-isopropyl-2,3,3,8-tetramethyl-4,7,13-trioxo-12-oxa-2,5,8-triazatetradecan-11-yl)thiazole-4-carboxylic acid (1.90 g, 3.61 mmol, 1.0 eq.) in dichloromethane (70 mL) was added pentafluorophenol (1.00 g, 5.43 mmol, 1.5 eq.) and N,N′-diisopropylcarbodiimide (512 mg, 3.96 mmol, 1.1 eq.) at 0° C. The reaction mixture was warmed to room temperature and stirred overnight. After the solvent was removed under reduced pressure, the reaction mixture was diluted with EtOAc (80 mL) then filtered over Celite. The filtrate was concentrated and purified on SiO₂ column chromatography (1:10 to 1:3 EtOAc/DCM) to afford the title compound (2.09 g, 84% yield), which was used directly for the next step. MS ESI m/z calcd for C₃₁H₄₂F₅N₄O₆S [M+H]⁺ 693.27. found 693.60.

### Example 139. Synthesis of tert-butyl 2-(triphenylphosphoranylidene)propanoate

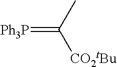

A mixture of tert-butyl-2-bromopropanoate (15.5 g, 74.1 mmol, 1.0 eq.) and triphenyl phosphine (19.4 g, 74.1 mmol, 1.0 eq.) in dry acetonitrile (45 mL) was stirred at room temperature for 18 h. Acetonitrile was removed under reduced pressure and toluene was added to crash out a white precipitate. Toluene was then decanted off and the white solid was dissolved in dichloromethane (100 mL) and transferred to a separatory funnel. 10% NaOH (100 mL) was added to the funnel, and the organic layer immediately turned yellow after shaking. The organic layer was separated and the aqueous layer was extracted with dichloromethane (30 mL) once. The dichloromethane layers were combined and washed with brine (50 mL) once, then dried over Na₂SO₄, filtered and concentrated, giving the ylide as a yellow solid (16.8 g, 58%).

### Example 140. Synthesis of (S)-methyl 3-(4-(benzyloxy)phenyl)-2-((tert-butoxy carbonyl)amino)propanoate

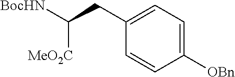 [SMILES: COC(=O)[C@H](Cc1ccc(OCc2ccccc2)cc1)NC(=O)OC(C)(C)C]

To a mixture of Boc-L-Tyr-OMe (20.0 g, 67.7 mmol, 1.0 eq.), K₂CO₃ (14.0 g, 101.6 mmol, 1.5 eq.) and KI (1.12 g, 6.77 mmol, 0.1 eq.) in acetone (100 mL) was added BnBr (10.5 mL, 81.3 mmol, 1.2 eq.) slowly. The mixture was then refluxed overnight. Water (250 mL) was added and the reaction mixture was extracted with EtOAc (3×100 mL). The combined organic layers were washed with brine (300 mL), dried over anhydrous Na₂SO₄, filtered, concentrated and purified by SiO₂ column chromatography (4:1 hexanes/EtOAc) to give a white solid title compound (26.12 g, 99% yield). ¹H NMR (500 MHz, CDCl₃) δ 7.44-7.41 (m, 2H), 7.41-7.36 (m, 2H), 7.35-7.30 (m, 1H), 7.04 (d, J=8.5 Hz, 2H), 6.93-6.89 (m, 2H), 5.04 (s, 2H), 4.97 (d, J=7.7 Hz, 1H), 4.55 (d, J=6.9 Hz, 1H), 3.71 (s, 3H), 3.03 (dd, J=14.4, 5.7 Hz, 2H), 1.44 (d, J=18.6 Hz, 10H). MS ESI m/z calcd for C₂₂H₂₇NO₅Na [M+Na]⁺408.18. found 408.11.

### Example 141. Synthesis of (S)-tert-butyl (1-(4-(benzyloxy)phenyl)-3-oxopropan-2-yl)carbamate

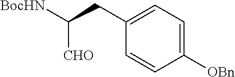 [SMILES: CC(C)(C)OC(=O)N[C@H](C=O)Cc1ccc(OCc2ccccc2)cc1]

To a solution of (S)-methyl 3-(4-(benzyloxy)phenyl)-2-((tert-butoxy carbonyl)amino)-propanoate (26.1 g, 67.8 mmol, 1.0 eq.) in anhydrous dichloromethane (450 mL) at −78° C. was added DIBAL (1.0 M in hexanes, 163 mL, 2.2 eq.) in 1 h. The mixture was stirred at −78° C. for 3 h and then quenched with 50 mL of ethanol. 1N HCl was added dropwise until pH 4 was reached. The resulting mixture was allowed to warm to 0° C. Layers were separated and the aqueous layer was further extracted with EtOAc (3×100 mL). The combined organic solution was washed with brine, dried over anhydrous Na₂SO₄, and concentrated. Trituration with PE/EtOAc and filtration gave a white solid title compound (18.3 g, 76% yield). MS ESI m/z calcd for C₂₂H₂₇NO₅Na [M+Na]⁺378.11. found 378.11.

### Example 142. Synthesis of (S,Z)-tert-butyl 5-(4-(benzyloxy)phenyl)-4-((tert-butoxycarbonyl)amino)-2-methylpent-2-enoate

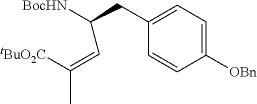 [SMILES: C/C(=C/[C@H](Cc1ccc(OCc2ccccc2)cc1)NC(=O)OC(C)(C)C)C(=O)OC(C)(C)C]

(S)-tert-Butyl (1-(4-(benzyloxy)phenyl)-3-oxopropan-2-yl)carbamate (0.84 g, 2 mmol, 1.0 eq.) was dissolved in dry dichloromethane (50 mL), to which tert-butyl 2-(triphenyl-phosphoranylidene)propanoate (1.6 g, 4 mmol, 2.0 eq.) was added and the solution was stirred at r.t. for 1.5 h as determined complete by TLC. Purification by column chromatography (10-50% EtOAc/hexanes) afforded the title compound (1.16 g, 98% yield).

### Example 143. Synthesis of (4R)-tert-butyl 4-((tert-butoxycarbonyl)amino)-5-(4-hydroxyphenyl)-2-methylpentanoate

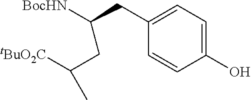 [SMILES: CC(C[C@H](Cc1ccc(O)cc1)NC(=O)OC(C)(C)C)C(=O)OC(C)(C)C]

(S,Z)-tert-Butyl 5-(4-(benzyloxy)phenyl)-4-((tert-but oxycarbonyl)amino)-2-methylpent-2-enoate (467 mg, 1 mmol) was dissolved in methanol (30 mL) and hydrogenated (1 atm) with Pd/C catalyst (10 wt %, 250 mg) at r.t. overnight. The catalyst was filtered off and the filtrate were concentrated under reduced pressure to afford the title compound (379 mg, 99% yield).

### Example 144. Synthesis of (4R)-tert-butyl 4-((tert-butoxycarbonyl)amino)-5-(4-hydroxy-3-nitrophenyl)-2-methylpentanoate

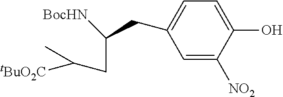 [SMILES: CC(C[C@H](Cc1ccc(O)c([N+](=O)[O-])c1)NC(=O)OC(C)(C)C)C(=O)OC(C)(C)C]

(4R)-tert-Butyl 4-((tert-butoxycarbonyl)amino)-5-(4-hydroxyphenyl)-2-methylpentanoate (379 mg, 1 mmol, 1.0 eq.) was dissolved in THF (20 mL), to which a solution of tert-butyl nitrite (315 mg, 3 mmol, 3.0 eq.) in THF (2 mL) was added. The reaction was stirred at r.t. for 3 h and then poured onto water, extracted with EtOAc (2×50 mL) and the combined organic phases were washed with brine (50 mL), dried over anhydrous Na₂SO₄, filtered and concentrated. Purification by column chromatography (10-50% EtOAc/hexanes) afforded the title compound (300 mg, 71% yield).

### Example 145. Synthesis of (4R)-tert-butyl 5-(3-amino-4-hydroxyphenyl)-4-((tert-butoxycarbonyl)amino)-2-methylpentanoate

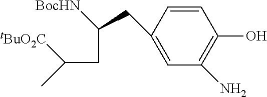 [SMILES: CC(C[C@H](Cc1ccc(O)c(N)c1)NC(=O)OC(C)(C)C)C(=O)OC(C)(C)C]

(4R)-Tert-butyl 4-((tert-butoxycarbonyl)amino)-5-(4-hydroxy-3-nitrophenyl)-2-methylpentanoate (200 mg, 0.47 mmol) was dissolved in EtOAc (30 mL) and mixed with palladium catalyst (10% on carbon, 100 mg), then hydrogenated (1 atm) at r.t. for 2 h. The catalyst was filtered off and all volatiles were removed under vacuum, which afforded the title compound (185 mg, 99%).

Alternatively, (4R)-tert-butyl 4-((tert-butoxycarbonyl)amino)-5-(4-hydroxy-3-nitrophenyl)-2-methylpentanoate (56 mg, 0.132 mmol) was dissolved in EtOAc (20 mL) and mixed with Pd/C catalyst (10 wt %, 50 mg) and hydrogenated (1 atm) at r.t. for 3 h. The catalyst was filtered off and all volatiles were removed under vacuum to afford the title compound (52 mg, 99% yield). MS ESI m/z calcd for C₂₁H₃₅N₂O₅ [M+H]⁺ 395.25. found 395.26.

### Example 146. Synthesis of (4R)-tert-butyl 4-((tert-butoxycarbonyl)amino)-5-(4-((tert-butyldimethylsilyl)oxy)-3-nitrophenyl)-2-methylpentanoate

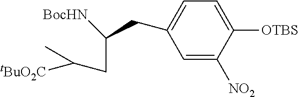 [SMILES: CC(C[C@H](Cc1ccc(O[Si](C)(C)C(C)(C)C)c([N+](=O)[O-])c1)NC(=O)OC(C)(C)C)C(=O)OC(C)(C)C]

To a solution of (4R)-tert-butyl 4-((tert-butoxycarbonyl)amino)-5-(4-hydroxy-3-nitrophenyl)-2-methylpentanoate (424 mg, 1 mmol) in DCM (20 mL), imidazole (408 mg, 6 mmol) and tert-butylchlorodimethylsilane (602 mg, 4 mmol) were added. The resulting solution was stirred at r.t. for 3 h. Afterwards, the reaction mixture was washed with brine (50 mL), dried over anhydrous Na₂SO₄, concentrated and purified by column chromatography (10% to 30% EtOAc/hexanes) to yield the title compound (344 mg, 64% yield).

### Example 147. Synthesis of (4R)-tert-butyl 5-(3-amino-4-((tert-butyldimethylsilyl) oxy)phenyl)-4-((tert-butoxycarbonyl)amino)-2-methylpentanoate

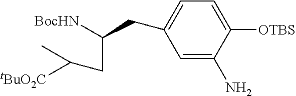 [SMILES: CC(C[C@H](Cc1ccc(O[Si](C)(C)C(C)(C)C)c(N)c1)NC(=O)OC(C)(C)C)C(=O)OC(C)(C)C]

(4R)-tert-Butyl 4-((tert-butoxycarbonyl)amino)-5-(4-((tert-butyldimethylsilyl)oxy)-3-nitrophenyl)-2-methylpentanoate (200 mg, 0.37 mmol) was dissolved in EtOAc (30 mL), mixed with palladium catalyst (10 wt % on carbon, 100 mg) and hydrogenated (1 atm) at r.t. for 2 h. The catalyst was filtered off and all volatiles were removed under vacuum to afford the title compound (187 mg, 99% yield).

### Example 148. Synthesis of 2-(1-azido-14,17-dimethyl-12,15-dioxo-3,6,9-trioxa-13,16-diazaoctadecanamido)-4-((2R)-5-(tert-butoxy)-2-((tert-butoxycarbonyl)amino)-4-methyl-5-oxopentyl)phenyl 1-azido-14,17-dimethyl-12,15-dioxo-3,6,9-trioxa-13,16-diazaoctadecan-18-oate

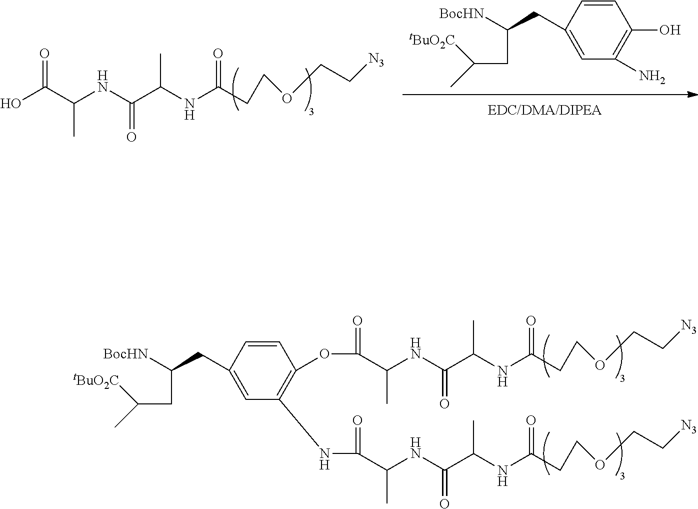

To a solution of 1-azido-14,17-dimethyl-12,15-dioxo-3,6,9-trioxa-13,16-diazaoctadecan-18-oic acid (1.50 g, 3.85 mmol) and (4R)-tert-butyl 5-(3-amino-4-hydroxyphenyl)-4-((tert-butoxycarbonyl)amino)-2-methylpentanoate (0.75 g, 1.90 mmol) in DMA (40 ml) was added EDC (2.05 g, 10.67 mmol) and DIPEA (0.70 ml, 4.0 mmol). The mixture was stirred for overnight, concentrated and purified on SiO₂ column eluted with EtOAc/CH₂Cl₂ (1:5 to 1:1) to afford the title compound (2.01 g, 82% yield, ˜95% pure by HPLC). MS ESI m/z calcd for C₅₁H₈₅N₁₂O₁₇ [M+H]⁺ 1137.61. found 1137.90.

### Example 149. Synthesis of (4R)-tert-butyl 5-(22,23-bis(2,5-dioxo-2,5-dihydro-1H-pyrrol-1-yl)-3,6,39,42-tetramethyl-2,5,8,21,24,37,40,43-octaoxo-3,4,5,6,7,8,9,10,12,13,15,16,18,19,20,21,22,23,24,25,26,27,29,30,32,33,35,36,37,38,39,40,41,42,43,44-hexatriacontahydro-2H-benzo[b][1,14,17,20,31,34,37,4,7,10,23,28,41,44]heptaoxa-heptaazacyclohexatetracontin-46-yl)-4-((tert-butoxycarbonyl)amino)-2-methylpentanoate

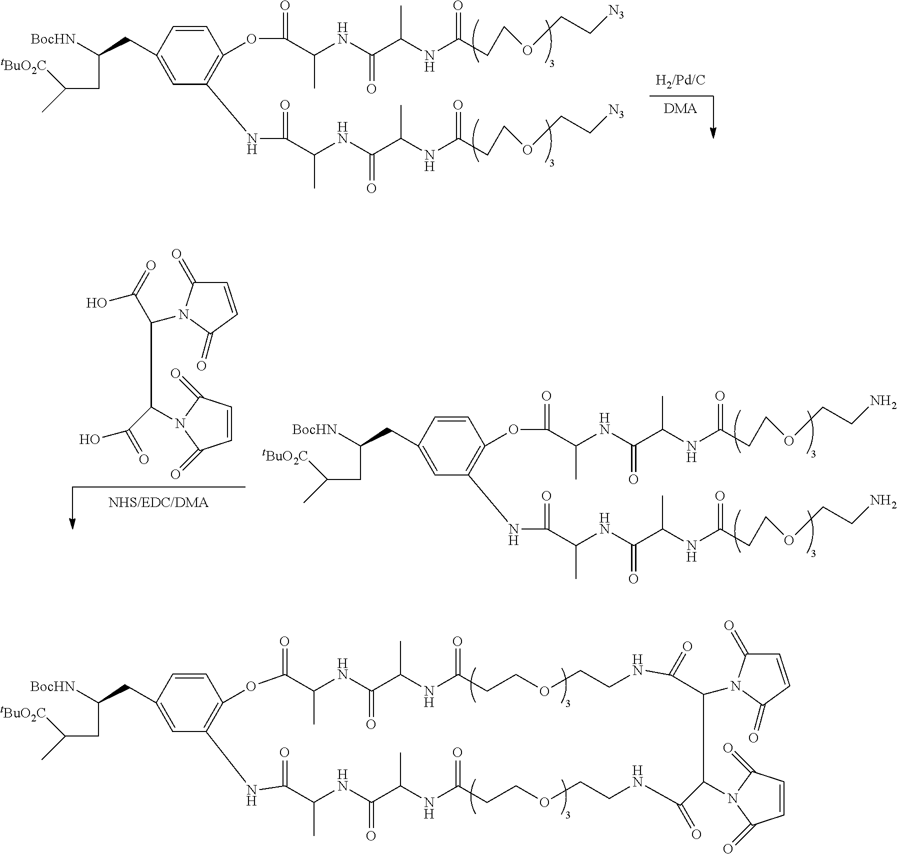

2-(1-Azido-14,17-dimethyl-12,15-dioxo-3,6,9-trioxa-13,16-diazaoctadecanamido)-4-((2R)-5-(tert-butoxy)-2-((tert-butoxycarbonyl)amino)-4-methyl-5-oxopentyl)phenyl 1-azido-14,17-dimethyl-12,15-dioxo-3,6,9-trioxa-13,16-diazaoctadecan-18-oate (900 mg, 0.79 mmol) was dissolved in EtOAc (30 mL), mixed with palladium catalyst (10 wt % on carbon, 100 mg) and hydrogenated (1 atm) at r.t. for 4 h. The catalyst was filtered off and all volatiles were removed under vacuum to afford 2-(1-amino-14,17-dimethyl-12,15-dioxo-3,6,9-trioxa-13,16-diazaoctadecanamido)-4-((2R)-5-(tert-butoxy)-2-((tert-butoxycarbonyl)amino)-4-methyl-5-oxopentyl)phenyl 1-amino-14,17-dimethyl-12,15-dioxo-3,6,9-trioxa-13,16-diazaoctadecan-18-oate (815 mg, 96% yield) which was used immediately without further purification. MS ESI m/z calcd for C₅₁H₈₈N₈O₁₇ [M+H]⁺ 1085.62. found 1085.95.

The diamino compound (810 mg, 0.75 mmol) and 2,3-bis(2,5-dioxo-2,5-dihydro-1H-pyrrol-1-yl)succinic acid (231 mg, 0.75 mmol) in DMA (10 ml) was added EDC (1.25 g, 6.51 mmol) and DIPEA (0.35 ml, 2.0 mmol). The mixture was stirred for overnight, concentrated and purified on SiO₂ column eluted with EtOAc/CH₂Cl₂ (1:5 to 1:1) to afford the title compound (844 mg, 83% yield, ˜95% pure by HPLC). MS ESI m/z calcd for C₆₃H₉₂N₁₀O₂₃ [M+H]⁺ 1357.63. found 1357.95.

### Example 150. Synthesis of (2R)-1-(22,23-bis(2,5-dioxo-2,5-dihydro-1H-pyrrol-1-yl)-3,6,39,42-tetramethyl-2,5,8,21,24,37,40,43-octaoxo-3,4,5,6,7, 8,9,10,12,13,15,16,18,19,20,21,22,23,24,25,26,27,29,30,32,33,35,36,37,38,39,40,41,42,43,44-hexatriacontahydro-2H-benzo[b][1,14,17,20,31,34,37,4,7,10,23,28,41,44]heptaoxaheptaazacyclohexatetracontin-46-yl)-4-carboxypentan-2-aminium

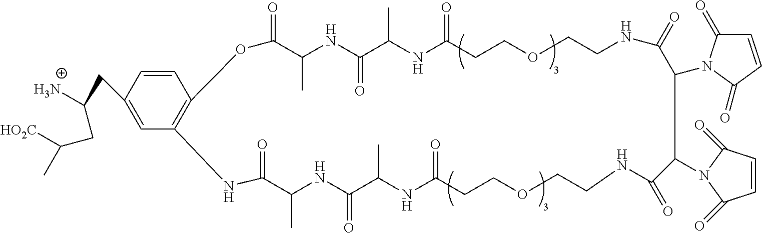

(4R)-Tert-butyl 5-(22,23-bis(2,5-dioxo-2,5-dihydro-1H-pyrrol-1-yl)-3,6,39,42-tetramethyl-2,5,8,21,24,37,40,43-octaoxo-3,4,5,6,7,8,9,10,12,13,15,16,18,19,20,21,22,23,24,25,26,27,29,30,32,33,35,36,37,38,39,40,41,42,43,44-hexatriacontahydro-2H-benzo[b][1,14,17,20,31,34,37,4,7,10,23,28,41,44]heptaoxa-heptaazacyclohexatetracontin-46-yl)-4-((tert-butoxycarbonyl)-amino)-2-methylpentanoate (840 mg, 0.62 mmol) was dissolved in the mixture of CH₂Cl₂ (6 ml) and TFA (4 ml). The mixture was stirred for overnight, diluted with toluene (10 ml), concentrated to afford the title compound (7.43 g, 100% yield, ˜91% pure by HPLC) which was used for the next step without further purification. MS ESI m/z calcd for C₅₄H₇₆N₁₀O₂₁ [M+H]⁺ 1200.51. found 1200.95.

### Example 151. Synthesis of (4R)-4-(2-((1R,3R)-1-acetoxy-3-((2S,3S)—N,3-dimethyl-2-((R)-1-methylpiperidine-2-carboxamido)pentanamido)-4-methylpentyl)thiazole-4-carboxamido)-5-(3-(3-(2-(2-azidoethoxy)ethoxy)propanamido)-4-hydroxyphenyl)-2-methylpentanoic Acid

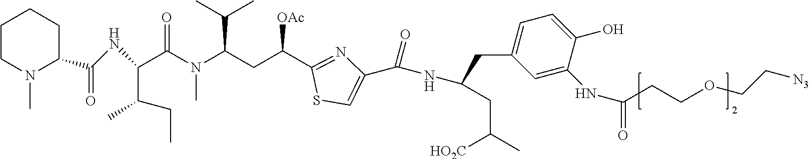

To a solution of (4R)-4-(2-((1R,3R)-1-acetoxy-3-((2S,3S)—N,3-dimethyl-2-((R)-1-methylpiperidine-2-carboxamido)pentanamido)-4-methylpentyl)thiazole-4-carboxamido)-5-(3-amino-4-hydroxyphenyl)-2-methylpentanoic acid (Huang Y. et al, Med Chem. #44, 249^{th} ACS National Meeting, Denver, Colo., Mar. 22˜26, 2015; WO2014009774) (100 mg, 0.131 mmol) in the mixture of DMA (10 ml) and NaH₂PO₄ buffer solution (pH 7.5, 1.0 M, 0.7 ml) was added 2,5-dioxopyrrolidin-1-yl 3-(2-(2-azidoethoxy)ethoxy)propanoate (80.0 mg, 0.266 mmol) in four portions in 2 h. The mixture was stirred overnight, concentrated and purified on C₁₈ preparative HPLC (3.0×25 cm, 25 ml/min), eluted with from 80% water/methanol to 10% water/methanol in 45 min to afford the title compound (101.5 mg, 82% yield). LC-MS (ESI) m/z calcd. for C₄₅H7₀N90₁₁S [M+H]⁺: 944.48, found: 944.70.

### Example 152. Synthesis of (4R)-4-(2-((1R,3R)-1-acetoxy-3-((2S,3S)—N,3-dimethyl-2-((R)-1-methyl-piperidine-2-carboxamido)pentanamido)-4-methylpentyl)thiazole-4-carboxamido)-5-(3-(3-(2-(2-aminoethoxy)ethoxy)propanamido)-4-hydroxyphenyl)-2-methylpentanoic Acid

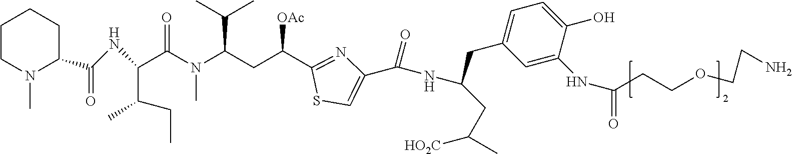

To a solution of (4R)-4-(2-((1R,3R)-1-acetoxy-3-((2S,3S)—N,3-dimethyl-2-((R)-1-methylpiperidine-2-carboxamido)pentanamido)-4-methylpentyl)thiazole-4-carboxamido)-5-(3-(3-(2-(2-azidoethoxy)ethoxy)propanamido)-4-hydroxyphenyl)-2-methylpentanoic acid (100.0 mg, 0.106 mmol) in methanol (25 ml) containing 0.1% HCl in a hydrogenation bottle was added Pd/C (25 mg, 10% Pd, 50% wet). After air was vacuumed out in the vessel and 35 psi H₂ was conducted in, the mixture was shaken for 4 h, filtered through Celite. The filtrate was concentrated and purified on C₁₈ preparative HPLC (3.0×25 cm, 25 ml/min), eluted with from 85% water/methanol to 15% water/methanol in 45 min to afford the title compound (77.5 mg, 79% yield). LC-MS (ESI) m/z calcd. for C₄₅H₇₂N₇O₁₁S [M+H]⁺: 918.49, found: 918.60.

### Example 153. Synthesis of (4R)-tert-butyl 5-(4-acetoxy-3-nitrophenyl)-4-((tert-butoxycarbonyl)amino)-2-methylpentanoate

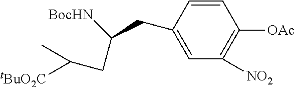 [SMILES: CC(=O)Oc1ccc(C[C@@H](CC(C)C(=O)OC(C)(C)C)NC(=O)OC(C)(C)C)cc1[N+](=O)[O-]]

To a solution of compound 190 (107.1 mg, 0.252 mmol) in dichloromethane (4.0 mL) at 0° C. was added acetic anhydride (0.11 mL, 1.17 mmol) and triethylamine (0.16 mL) in sequence. The reaction was then warmed to r.t. and stirred for 1 h, diluted with dichloromethane and washed with water and brine, dried over anhydrous Na₂SO₄, filtered and concentrated. The residue was purified by column chromatography (0-15% EA/PE) to give a colorless oil (120.3 mg, theoretical yield). MS ESI m/z calcd for C₂₃H₃₅N₂O₈ [M+H]⁺ 467.23, found 467.23.

### Example 154. Synthesis of (4R)-tert-butyl 5-(4-acetoxy-3-aminophenyl)-4-((tert-butoxycarbonyl)amino)-2-methylpentanoate

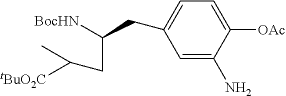 [SMILES: CC(=O)Oc1ccc(C[C@@H](CC(C)C(=O)OC(C)(C)C)NC(=O)OC(C)(C)C)cc1N]

(4R)-Tert-butyl 5-(4-acetoxy-3-nitrophenyl)-4-((tert-butoxycarbonyl)amino)-2-methylpentanoate (120.3 mg, 0.258 mmol) was dissolved in ethyl acetate (5 mL) and acetic acid (0.5 mL). To which Pd/C (10 wt %, 10 mg) was added and the mixture was stirred under H₂ balloon at r.t. for 30 min before filtration through a Celite pad with washing of the pad with ethyl acetate. The filtrate was concentrated and purified by column chromatography (0-25% EA/PE) to give a yellow oil (120.9 mg, theoretical yield). MS ESI m/z calcd for C₂₃H₃₇N₂O₆ [M+H]⁺ 437.26. found 437.28.

### Example 155. Synthesis of (4R)-ethyl 5-(3-(4-(((benzyloxy)carbonyl)amino) butanamido)-4-((tert-butyldimethylsilyl)oxy)phenyl)-4-((tert-butoxycarbonyl)amino)-2-methylpentanoate

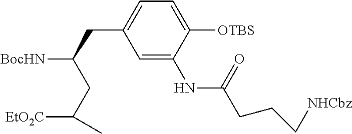 [SMILES: CCOC(=O)C(C)C[C@H](Cc1ccc(O[Si](C)(C)C(C)(C)C)c(NC(=O)CCCNC(=O)OCc2ccccc2)c1)NC(=O)OC(C)(C)C]

2,5-dioxopyrrolidin-1-yl 4-(((benzyloxy)carbonyl)amino)butanoate (0.396 g, 1.2 mmol) and (4R)-ethyl 5-(3-amino-4-hydroxyphenyl)-4-((tert-butoxycarbonyl) amino)-2-methylpentanoate (0.44 g, 1.2 mmol) were dissolved in EtOH (10 mL), and phosphate buffer solution (pH=7.5, 0.1M, 2 ml) was added. The reaction mixture was stirred at r.t. overnight and then the solvent was removed under reduced pressure and the residue purified by SiO₂ column chromatography to give the title product (0.485 g, 70%). ESI: m/z: calcd for C₃₁H₄₄N₃O₈ [M+H]⁺:586.31. found 586.31.

### Example 156. Synthesis of (4R)-ethyl 5-(3-(4-aminobutanamido)-4-((tert-butyl dimethylsilyl)oxy)phenyl)-4-((tert-butoxycarbonyl)amino)-2-methylpentanoate

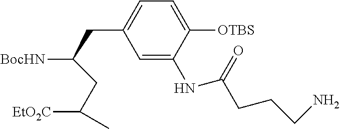 [SMILES: CCOC(=O)C(C)C[C@H](Cc1ccc(O[Si](C)(C)C(C)(C)C)c(NC(=O)CCCN)c1)NC(=O)OC(C)(C)C]

(4R)-ethyl 5-(3-(4-(((benzyloxy)carbonyl)amino) butanamido)-4-((tert-butyldimethyl-silyl)oxy)phenyl)-4-((tert-butoxycarbonyl)amino)-2-methylpentanoate (0.35 g, 0.5 mmol) was dissolved in MeOH (5 ml), and Pd/C (10 wt %, 35 mg) was then added. The reaction mixture was stirred at r.t. under H₂ balloon overnight, then filtered through Celite and the filtrate was concentrated under reduced pressure to give the title product (0.22 g, 79% yield). ESI MS m/z: calcd for C₂₉H₅₂N₃O₆Si [M+H]⁺:566.35. found 566.35.

### Example 157. Synthesis of 2-((6S,9S,12R,14R)-9-((S)-sec-butyl)-14-hydroxy-6,12-diisopropyl-2,2,5,11-tetramethyl-4,7,10-trioxo-3-oxa-5,8,11-triazatetradecan-14-yl)thiazole-4-carboxylic Acid

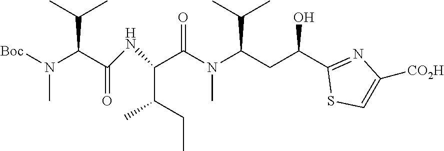 [SMILES: CC[C@H](C)[C@H](NC(=O)[C@H](C(C)C)N(C)C(=O)OC(C)(C)C)C(=O)N(C)[C@H](C[C@@H](O)c1nc(C(=O)O)cs1)C(C)C]

To a solution of Boc-N-Me-L-Val-OH (33 mg, 0.14 mmol) in EtOAc was added pentafluorophenol (39 mg, 0.21 mmol) and DCC (32 mg, 0.154 mmol). The reaction mixture was stirred at r.t. for 16 h and then filtered over a Celite pad, with washing of the pad with EtOAc. The filtrate was concentrated and re-dissolved in DMA (2 mL), and then 2-((1R,3R)-3-((2S,3S)-2-amino-N,3-dimethylpentanamido)-1-hydroxy-4-methylpentyl)thiazole-4-carboxylic acid (52 mg, 0.14 mmol) and DIPEA (48.5 μL, 0.28 mmol) were added. The reaction mixture was stirred at r.t. for 24 h and then concentrated and purified by reverse phase HPLC (C₁₈ column, 10-100% acetonitrile/water) to afford the title compound (40.2 mg, 49% yield). ESI MS m/z: calcd for C₂₈H₄₉N₄O₇S [M+H]⁺: 585.32. found 585.32.

### Example 158. Synthesis of 2-((6S,9S,12R,14R)-9-((S)-sec-butyl)-6,12-di-isopropyl-2,2,5,11-tetramethyl-4,7,10,16-tetraoxo-3,15-dioxa-5,8,11-triazaheptadecan-14-yl)thiazole-4-carboxylic Acid

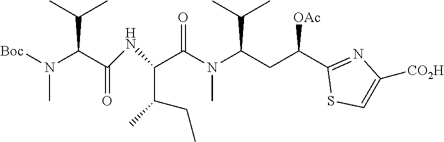 [SMILES: CC[C@H](C)[C@H](NC(=O)[C@H](C(C)C)N(C)C(=O)OC(C)(C)C)C(=O)N(C)[C@H](C[C@@H](OC(C)=O)c1nc(C(=O)O)cs1)C(C)C]

2-((6S,9S,12R,14R)-9-((S)-sec-butyl)-14-hydroxy-6,12-diisopropyl-2,2,5,11-tetramethyl-4,7,10-trioxo-3-oxa-5,8,11-triazatetradecan-14-yl)thiazole-4-carboxylic acid (40 mg, 0.069 mmol) was dissolved in pyridine (8 mL), to which acetic anhydride (20.4 mg, 0.2 mmol) was added at 0° C. and the reaction was allowed to warm to r.t. and stirred overnight. The mixture was concentrated and the residue purified by SiO₂ column chromatography with a gradient of DCM/MeOH to give the title product (48.1 mg, ˜100% yield). ESI MS m/z: calcd for C₃₀H₅₁N₄O₈S [M+H]⁺ 627.33. found 627.33.

### Example 159. Synthesis of (4R)-4-(2-((6S,9S,12R,14R)-9-((S)-sec-butyl)-6,12-diisopropyl-2,2,5,11-tetramethyl-4,7,10,16-tetraoxo-3,15-dioxa-5,8,11-triazaheptadecan-14-yl)thiazole-4-carboxamido)-2-methyl-5-phenylpentanoic Acid

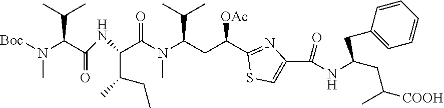 [SMILES: CC[C@H](C)[C@H](NC(=O)[C@H](C(C)C)N(C)C(=O)OC(C)(C)C)C(=O)N(C)[C@H](C[C@@H](OC(C)=O)c1nc(C(=O)N[C@@H](Cc2ccccc2)CC(C)C(=O)O)cs1)C(C)C]

To a solution of 2-((6S,9S,12R,14R)-9-((S)-sec-butyl)-6,12-di-isopropyl-2,2,5,11-tetramethyl-4,7,10,16-tetraoxo-3,15-dioxa-5,8,11-triazaheptadecan-14-yl)thiazole-4-carboxylic acid (48.1 mg, 0.077 mmol) in EtOAc was added pentafluorophenol (21.2 mg, 0.115 mmol) and DCC (17.4 mg, 0.085 mmol). The reaction mixture was stirred at r.t. for 16 h and then filtered over a Celite pad, with washing of the pad with EtOAc. The filtrate was concentrated and re-dissolved in DMA (4 mL), and then (4R)-4-amino-2-methyl-5-phenylpentanoic acid (20.7 mg, 0.1 mmol) and DIPEA (26.8 μL, 0.154 mmol) were added. The reaction mixture was stirred at r.t. for 24 h and then concentrated and purified by reverse phase HPLC (C₁₈ column, 10-100% acetonitrile/water) to afford the title compound (63 mg, ˜100% yield). ESI MS m/z: calcd for C₄₂H₆₆N₅O₉S [M+H]⁺ 816.45. found 816.45.

### Example 160. Synthesis of (4R)-4-(2-((3S,6S,9R,11R)-6-((S)-sec-butyl)-3,9-diisopropyl-8-methyl-4,7,13-trioxo-12-oxa-2,5,8-triazatetradecan-11-yl)thiazole-4-carboxamido)-2-methyl-5-phenylpentanoic Acid Hydrochloride Salt

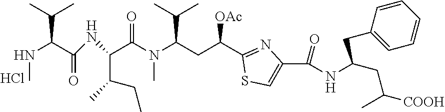 [SMILES: Cl ; CC[C@H](C)[C@H](NC(=O)[C@@H](NC)C(C)C)C(=O)N(C)[C@H](C[C@@H](OC(C)=O)c1nc(C(=O)N[C@@H](Cc2ccccc2)CC(C)C(=O)O)cs1)C(C)C]

(4R)-4-(2-((6S,9S,12R,14R)-9-((S)-sec-butyl)-6,12-diisopropyl-2,2,5,11-tetramethyl-4,7,10,16-tetraoxo-3,15-dioxa-5,8,11-triazaheptadecan-14-yl)thiazole-4-carboxamido)-2-methyl-5-phenylpentanoic acid (60 mg, 0.073 mmol) in ethyl acetate (3 ml) and hydrogen chloride (0.8 ml, 12 M). The mixture was stirred for 30 min and diluted with toluene (5 ml) and dioxane (5 ml). The mixture was evaporated and co-evaporated with dioxane (5 ml) and toluene (5 ml) to dryness. The yielded crude title product (57.1 mg, 103% yield) was used for the next step without further purification. ESI MS m/z: calcd for C₃₇H₅₈N₅O₇S [M+H]⁺ 716.40, found 716.60.

### Example 161. Synthesis of (4R)-tert-butyl-5-(3-(2-(2-(((benzyloxy)carbonyl)amino)-propanamido)acetamido)-4-hydroxyphenyl)-4-((tert-butoxycarbonyl)amino)-2-methylpentanoate

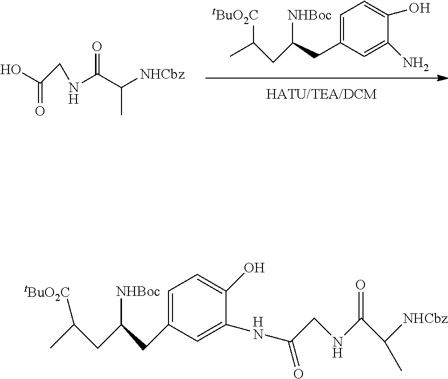 [SMILES: CC(NC(=O)OCc1ccccc1)C(=O)NCC(=O)O ; CC(C[C@H](Cc1ccc(O)c(N)c1)NC(=O)OC(C)(C)C)C(=O)OC(C)(C)C ; CC(C[C@H](Cc1ccc(O)c(NC(=O)CNC(=O)C(C)NC(=O)OCc2ccccc2)c1)NC(=O)OC(C)(C)C)C(=O)OC(C)(C)C]

2-(2-(((benzyloxy)carbonyl)amino)propanamido)acetic acid (0.2 g, 0.7 mmol), (4R)-tert-butyl-5-(3-amino-4-hydroxyphenyl)-4-((tert-butoxycarbonyl)amino)-2-methylpentanoate (0.19 g, 0.48 mmol), and HATU (0.18 g, 0.48 mmol) were dissolved in DCM (20 ml), followed by addition of TEA (134 ul, 0.96 mmol). The reaction mixture was stirred at RT overnight, concentrated under reduced pressure and the residue was purified on SiO₂ column to give the title product (0.3 g, 95%). ESI: m/z: calcd for C₃₄H₄₉N₄O₉ [M+H]⁺:657.34. found 657.34.

### Example 162. Synthesis of (4R)-tert-butyl-5-(3-(2-(2-aminopropanamido)acetamido)-4-hydroxyphenyl)-4-((tert-butoxycarbonyl)amino)-2-methylpentanoate

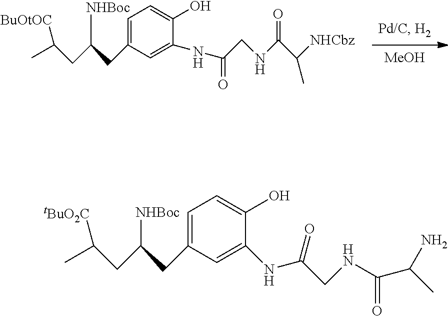

In a hydrogenation bottle, Pd/C (0.1 g, 33 wt %, 50% wet) was added to a solution of (4R)-tert-butyl-5-(3-(2-(2-(((benzyloxy)carbonyl)amino)propanamido)acetamido)-4-hydroxyphenyl)-4-((tert-butoxycarbonyl)amino)-2-methylpentanoate (0.3 g, 0.46 mmol) in MeOH (10 mL). The mixture was shaken overnight under 1 atm H₂ then filtered through Celite (filter aid), and the filtrate was concentrated to afford the title compound (0.21 g, 87%) used for next step without further purification. ESI: m/z: calcd for C₂₆H₄₃N₄O₇ [M+H]⁺:523.31. found 523.31.

### Example 163. Synthesis of B-1 (a Tubulysin Fragment Having a Bis-Linker)

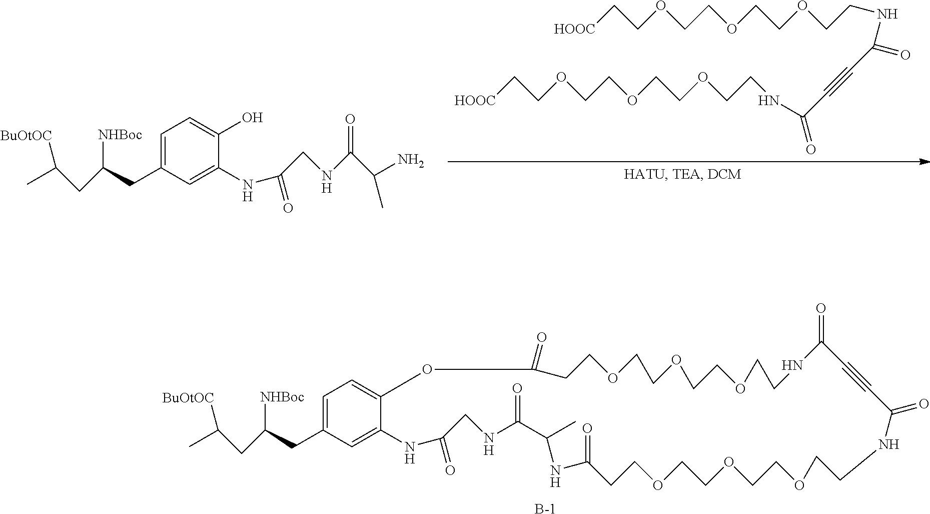

5-(3-(2-(2-Aminopropanamido)acetamido)-4-hydroxyphenyl)-4-((tert-butoxycarbonyl)amino)-2-methylpentanoate (0.11 g, 0.2 mmol), 4,17-dioxo-4,7,10,21,24,27-hexaoxa-13,18-diazatriacont-15-yne-1,30-dioic acid (0.104 g, 0.2 mmol), HATU (0.07 g, 0.2 mmol) were dissolved in DCM (10 ml), followed by addition of TEA (55 ul, 0.4 mmol). The reaction mixture was stirred at RT overnight, concentrated under reduced pressure and purified on SiO₂ column to give the product B-1 (0.046 g, 23%). ESI: m/z: calcd for C₄₈H₇₅N₆O₁₇ [M+H]⁺: 1007.51. found 1007.52.

### Example 164. Synthesis of B-2 (a Tubulysin Fragment Having a Bis-Linker)

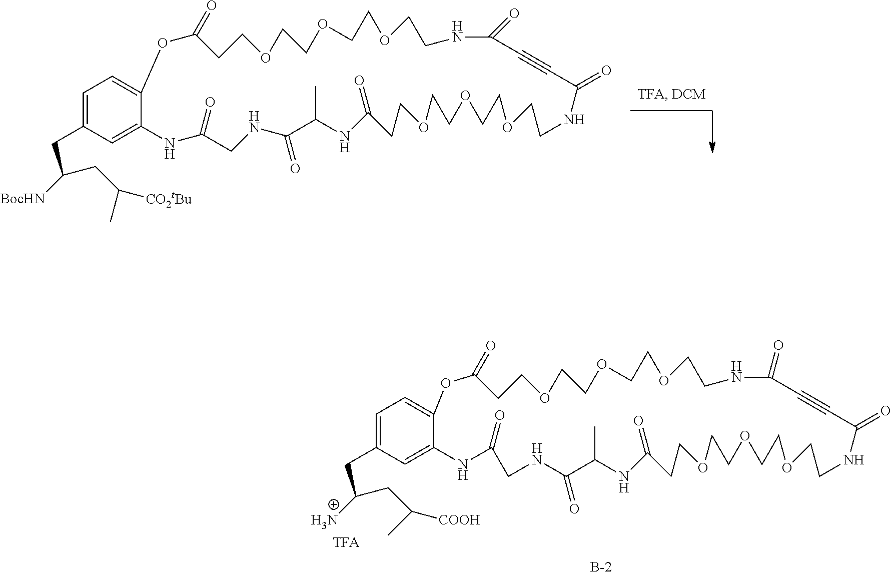

Compound B-1 (0.046 g, 0.045 mmol) dissolved in DCM (1 ml) was added TFA (1 ml) and the reaction mixture was stirred at RT for 2h, concentrated and co-evaporated with DCM/toluene to afford crude compound B-2 (38.6 mg, 100% yield) used for next step without further purification. ESI: m/z: calcd for C₃₉H₅₉N₆O₁₅ [M+H]⁺: 851.40. found 851.95.

### Example 165. Synthesis of B-3 (a Tubulysin Analog Having a Bis-Linker)

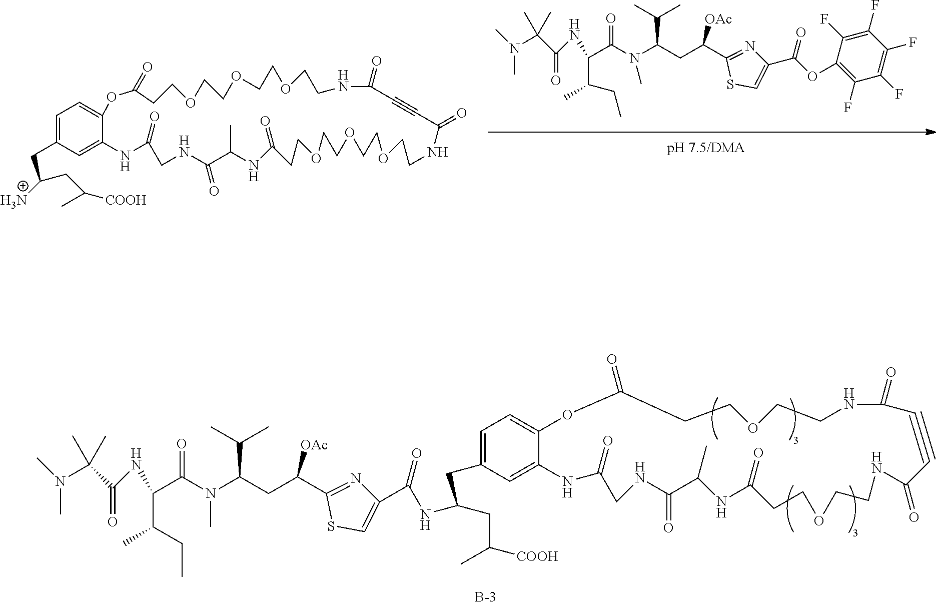

To the solution of compound B-2 (38.6 mg, 0,045 mmol) in DMA (4 ml) was added perfluorophenyl 2-((6S,9R,11R)-6-((S)-sec-butyl)-9-isopropyl-2,3,3,8-tetramethyl-4,7,13-trioxo-12-oxa-2,5,8-triazatetradecan-11-yl)thiazole-4-carboxylate (31.14 mg, 0.045 mmol), then DIPEA (28 ul, 0.159 mmol) was added, the reaction was stirred overnight. Then the solution was concentrated and purified by HPLC with a gradient of MeCN/H₂O (10% MeCN to 70% MeCN in 45 min, C-18 column, 10 mm (d)×250 mm (1), 9 ml/min) to give the title product (7.9 mg, 13%). ESI: m/z: calcd for C₆₄H₉₉N₁₀O₂₀S [M+H]⁺: 1359.67. found 1359.62.

### Example 166. Synthesis of (4R)-tert-butyl-5-(3-(2-(((benzyloxy)carbonyl)amino)-3-methylbutanamido)-4-hydroxyphenyl)-4-((tert-butoxycarbonyl)amino)-2-methylpentanoate

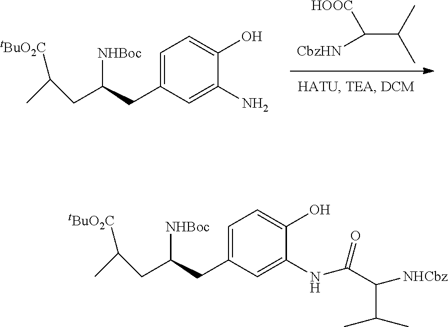 [SMILES: CC(C[C@H](Cc1ccc(O)c(N)c1)NC(=O)OC(C)(C)C)C(=O)OC(C)(C)C ; CC(C)C(NC(=O)OCc1ccccc1)C(=O)O ; CC(C[C@H](Cc1ccc(O)c(NC(=O)C(NC(=O)OCc2ccccc2)C(C)C)c1)NC(=O)OC(C)(C)C)C(=O)OC(C)(C)C]

(4R)-tert-butyl-5-(3-amino-4-hydroxyphenyl)-4-((tert-butoxycarbonyl)amino)-2-methylpentanoate (0.2 g, 0.51 mmol), 2-(((benzyloxy)carbonyl)amino)-3-methylbutanoic acid (0.13 g, 0.51 mmol), HATU (0.2 g, 0.51 mmol) were dissolved in DCM (20 ml), followed by TEA (110 ul, 0.8 mmol) was added. The reaction mixture was stirred at RT overnight. Then the solvent was removed under reduced pressure and purified by SiO₂ column to give the title product 12 (0.29 g, 90%). ESI: m/z: calcd for C₃₄H₅₀N₃O₈ [M+H]⁺: 628.35. found 628.35.

### Example 167. Synthesis of (4R)-tert-butyl-5-(3-(2-amino-3-methylbutanamido)-4-hydroxyphenyl)-4-((tert-butoxycarbonyl)amino)-2-methylpentanoate

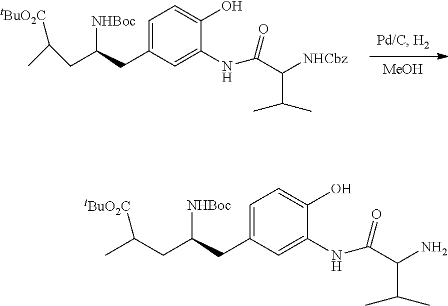

In a hydrogenation bottle, Pd/C (0.1 g, 33 wt %, 50% wet) was added to a solution (4R)-tert-butyl-5-(3-(2-(((benzyloxy)carbonyl)amino)-3-methylbutanamido)-4-hydroxyphenyl)-4-((tert-butoxycarbonyl)amino)-2-methylpentanoate (0.29 g, 0.46 mmol) in MeOH (10 mL). The mixture was shaken overnight under 1 atm H₂, then filtered through Celite (filter aid). The filtrate was concentrated to afford the title compound (0.23 g, 100%) and used for next step without further purification. ESI: m/z: calcd for C₂₆H₄₄N₃O₆ [M+H]⁺:494.64. found 494.64.

### Example 168. Synthesis of (4R)-tert-butyl-5-(3-(2-(2-(((benzyloxy)carbonyl)amino)propanamido)-3-methylbutanamido)-4-hydroxyphenyl)-4-((tert-butoxycarbonyl)amino)-2-methylpentanoate

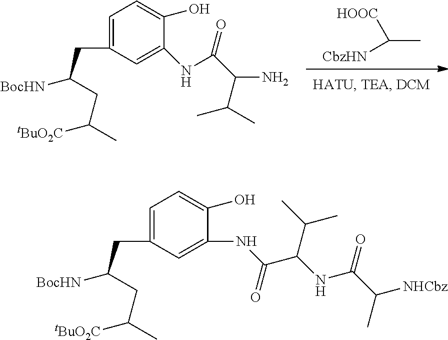 [SMILES: CC(C[C@H](Cc1ccc(O)c(NC(=O)C(N)C(C)C)c1)NC(=O)OC(C)(C)C)C(=O)OC(C)(C)C ; CC(NC(=O)OCc1ccccc1)C(=O)O ; CC(C[C@H](Cc1ccc(O)c(NC(=O)C(NC(=O)C(C)NC(=O)OCc2ccccc2)C(C)C)c1)NC(=O)OC(C)(C)C)C(=O)OC(C)(C)C]

(4R)-tert-butyl-5-(3-(2-amino-3-methylbutanamido)-4-hydroxyphenyl)-4-((tert-butoxycarbonyl)amino)-2-methylpentanoate (0.23 g, 0.46 mmol), 2-(((benzyloxy)carbonyl)amino-propanoic acid (0.10 g, 0.46 mmol) and HATU (0.18 g, 0.46 mmol) were dissolved in DCM (20 ml), followed by addition of TEA (110 ul, 0.8 mmol). The reaction mixture was stirred at RT overnight, concentrated under reduced pressure and purified on SiO₂ column to give the title product (0.3 g, 95%). ESI: m/z: calcd for C₃₇H₅₅N₄O₉ [M+H]⁺: 699.39. found 699.35.

### Example 169. Synthesis of (4R)-tert-butyl-5-(3-(2-(2-aminopropanamido)-3-methylbutanamido)-4-hydroxyphenyl)-4-((tert-butoxycarbonyl)amino)-2-methylpentanoate

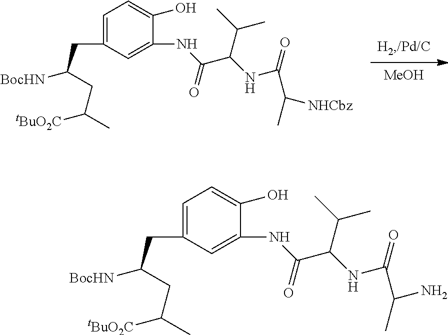 [SMILES: "MeOH" ; CC(C[C@H](Cc1ccc(O)c(NC(=O)C(NC(=O)C(C)NC(=O)OCc2ccccc2)C(C)C)c1)NC(=O)OC(C)(C)C)C(=O)OC(C)(C)C ; CC(N)C(=O)NC(C(=O)Nc1cc(C[C@@H](CC(C)C(=O)OC(C)(C)C)NC(=O)OC(C)(C)C)ccc1O)C(C)C]

In a hydrogenation bottle, Pd/C (0.1 g, 33 wt %, 50% wet) was added to a solution of (4R)-tert-butyl-5-(3-(2-(2-(((benzyloxy)carbonyl)amino)propanamido)-3-methylbutanamido)-4-hydroxyphenyl)-4-((tert-butoxycarbonyl)amino)-2-methylpentanoate (0.3 g, 0.43 mmol) in MeOH (10 mL). The mixture was shaken overnight under 1 atm H₂ then filtered through Celite (filter aid), the filtrate was concentrated to afford the title compound (0.22 g, 93%) which was used for the next step without further purification. ESI: m/z: calcd for C₂₉H₄₉N₄O₇ [M+H]⁺:565.35. found 565.31.

### Example 170. Synthesis of B-4 (a Tubulysin Fragment Having a Bis-Linker)

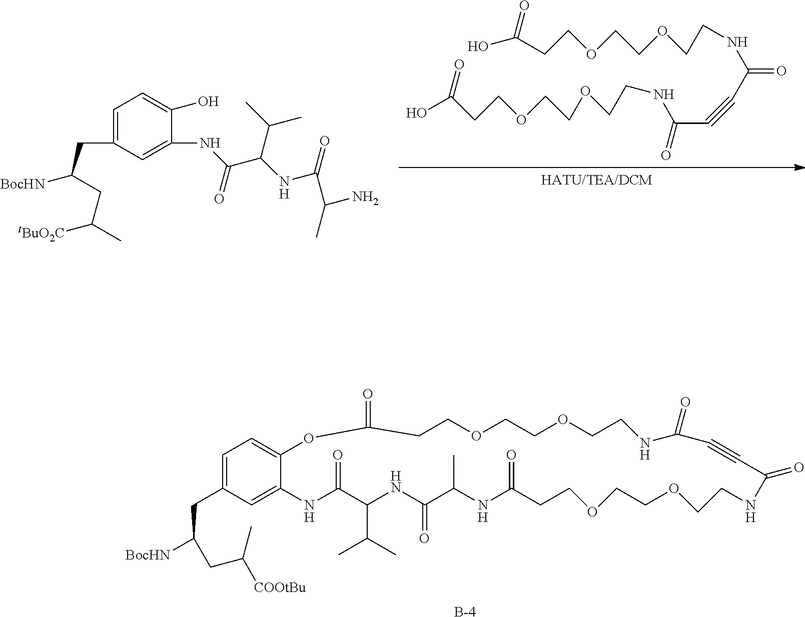

(4R)-tert-butyl-5-(3-(2-(2-aminopropanamido)-3-methylbutanamido)-4-hydroxyphenyl)-4-((tert-butoxycarbonyl)amino)-2-methylpentanoate (0.05 g, 0.09 mmol), 11,14-dioxo-4,7,18,21-tetraoxa-10,15-diazatetracos-12-yne-1,24-dioic acid (0.038 g, 0.09 mmol), HATU (0.067 g, 0.18 mmol) were dissolved in DCM (10 ml), followed by addition of TEA (55 ul, 0.4 mmol). The reaction mixture was stirred at RT overnight, concentrated under reduced pressure and purified on SiO₂ column to give the product B-4 (0.01 g, 12%). ESI: m/z: calcd for C₄₇H₇₃N₆O₁₅ [M+H]⁺: 961.51. found 961.52.

### Example 171. Synthesis of B-5 (a Tubulysin Fragment Having a Bis-Linker)

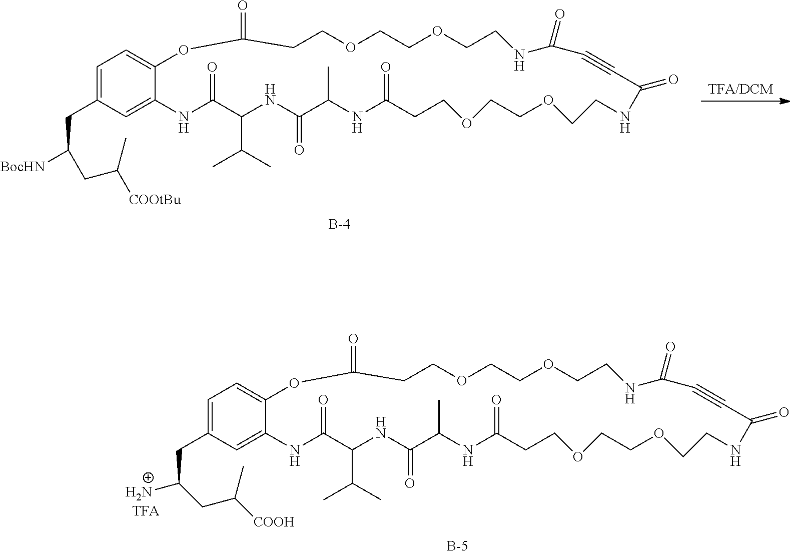

Compound B-4 (0.01 g, 0.01 mmol) was dissolved in DCM (1 ml), followed by addition of TFA (0.8 ml). The reaction mixture was stirred at RT for 2h, concentrated to afford compound B-5 (10 mg) for the next step without further purification. ESI: m/z: calcd for C₃₈H₅₆N₆O₁₃ [M+H]⁺: 804.39. found 804.65.

### Example 172. Synthesis of B-6 (a Tubulysin Analog Having a Bis-Linker)

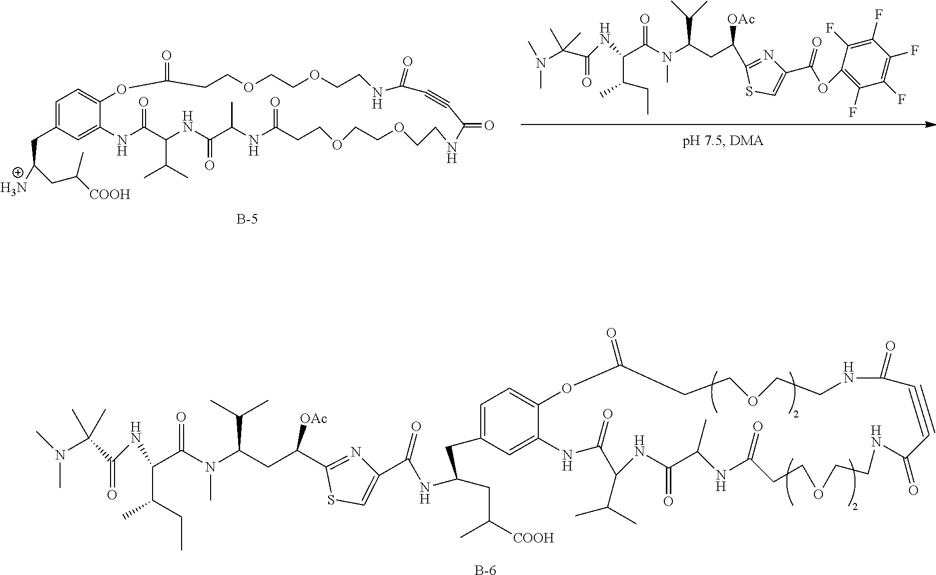

To the solution of compound B-5 (10 mg) in DMA (4 ml) was added pentafluo-actived acid compound (6.92 mg, 0.01 mmol) and DIPEA (3.4 ul, 0.02 mmol). The reaction mixture was stirred overnight, concentrated and purified on HPLC with a gradient of MeCN/H₂O (10% MeCN to 70% MeCN in 45 min, C-18 column, 10 mm (d)×250 mm (1), 9 ml/min) to give the product B-6 (8.1 mg, 62%). ESI: m/z: calcd for C₆₃H₉₇N₁₀O₁₈S [M+H]⁺: 1313.66. found 1313.66.

### Example 173. Synthesis of B-7 (a Tubulysin Fragment Having a Bis-Linker)

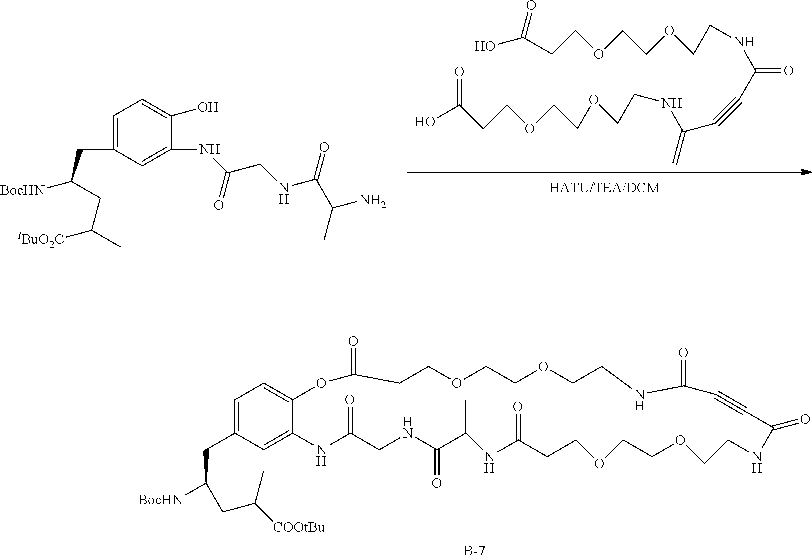

(4R)-tert-butyl-5-(3-(2-(2-aminopropanamido)acetamido)-4-hydroxyphenyl)-4-((tert-butoxycarbonyl)amino)-2-methylpentanoate (0.21 g, 0.4 mmol), 11,14-dioxo-4,7,18,21-tetraoxa-10,15-diazatetracos-12-yne-1,24-dioic acid (0.17 g, 0.4 mmol), HATU (0.15 g, 0.4 mmol) were dissolved in DCM (10 ml), followed by addition of TEA (110 ul, 0.8 mmol) The reaction mixture was stirred at RT overnight, concentrated under reduced pressure and purified on SiO₂ column to give the product B-7 (0.126 g, 34%). ESI: m/z: calcd for C₄₄H₆₇N₆O₁₅ [M+H]⁺: 919.46. found 919.46.

### Example 174. Synthesis of B-8 (a Tubulysin Fragment Having a Bis-Linker)

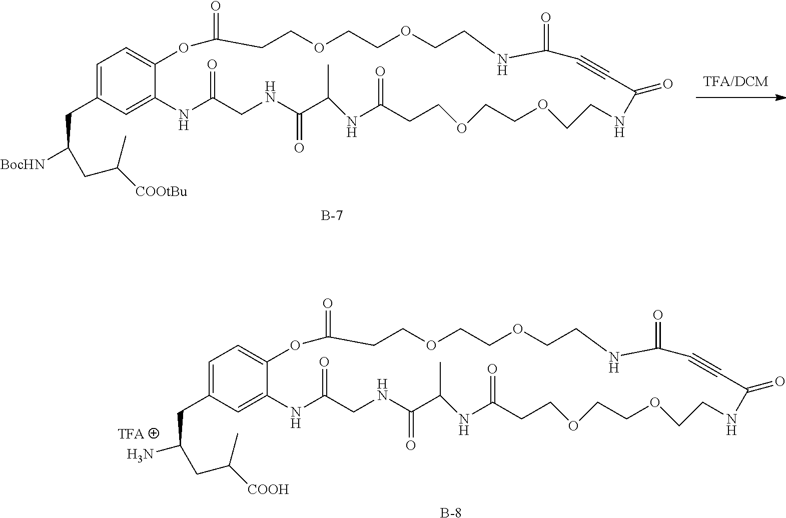

Compound B-7 (0.041 g, 0.045 mmol) was dissolved in DCM (1 ml), followed by addition of TFA (1 ml). The reaction mixture was stirred at RT for 2h, concentrated to afford compound B-8 which was used for next step without further purification. ESI: m/z: calcd for C₃₅H₅₁N₆O₁₃ [M+H]⁺: 763.35. found 763.80.

### Example 175. Synthesis of B-9 (a Tubulysin Analog Having a Bis-Linker)

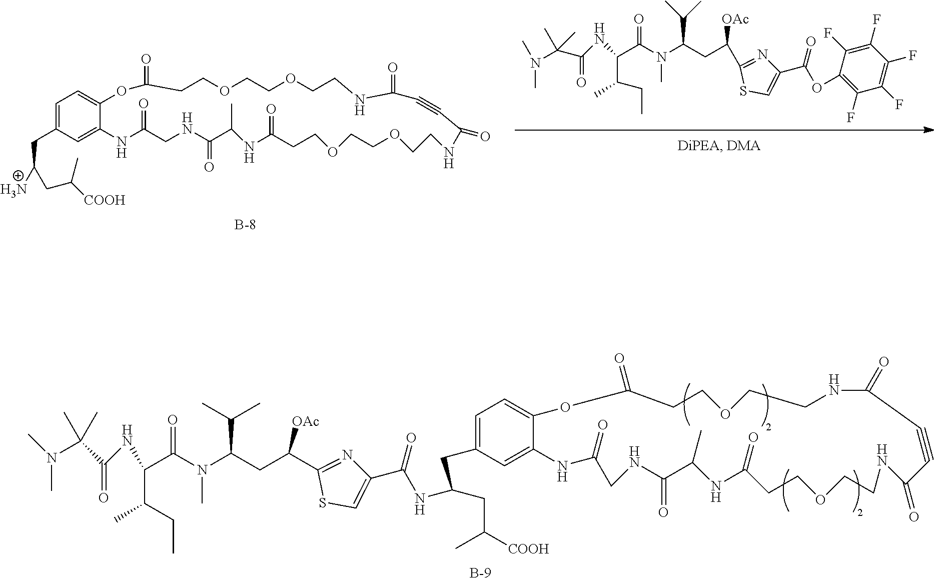

To the solution of compound B-8 (9.1 mg, 0.012 mmol) in DMA (1 ml) was added pentafluo-actived acid compound (8.3 mg, 0.012 mmol) and DIPEA (1.4 ul, 0.008 mmol). The reaction mixture was stirred overnight, concentrated and purified on HPLC with a gradient of MeCN/H₂O (10% MeCN to 70% MeCN in 45 min, C-18 column, 10 mm (d)×250 mm (1), 9 ml/min) to give the title B-9 (4.7 mg, 31%). ESI: m/z: calcd for C₆₀H₉₁N₁₀O₁₈S [M+H]⁺: 1271.62. found 1271.62.

### Example 176. Synthesis of (4R)-tert-butyl-5-(3-(2-(((benzyloxy)carbonyl)amino)-propanamido)-4-hydroxyphenyl)-4-((tert-butoxycarbonyl)amino)-2-methylpentanoate

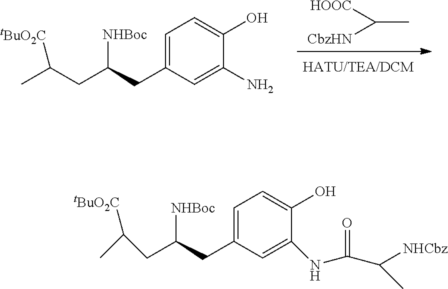 [SMILES: CC(C[C@H](Cc1ccc(O)c(N)c1)NC(=O)OC(C)(C)C)C(=O)OC(C)(C)C ; CC(NC(=O)OCc1ccccc1)C(=O)O ; CC(C[C@H](Cc1ccc(O)c(NC(=O)C(C)NC(=O)OCc2ccccc2)c1)NC(=O)OC(C)(C)C)C(=O)OC(C)(C)C]

(4R)-tert-butyl-5-(3-amino-4-hydroxyphenyl)-4-((tert-butoxycarbonyl)amino)-2-methylpentanoate (0.3 g, 0.76 mmol), 2-(((benzyloxy)carbonyl)amino-propanoic acid (0.17 g, 0.76 mmol), HATU (0.29 g, 0.76 mmol) were dissolved in DCM (20 ml), followed by addition of TEA (110 ul, 0.8 mmol). The reaction mixture was stirred at RT overnight, concentrated under reduced pressure and purified on SiO₂ column to give the title product (0.43 g, 95%). ESI: m/z: calcd for C₃₂H₄₆N₃O₈ [M+H]⁺: 600.32. found 600.32.

### Example 177. Synthesis of (4R)-tert-butyl-5-(3-(2-aminopropanamido)-4-hydroxyphenyl)-4-((tert-butoxycarbonyl)amino)-2-methylpentanoate

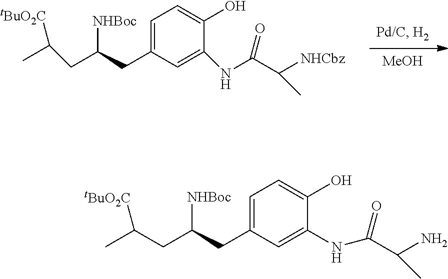 [SMILES: "MeOH" ; CC(C[C@H](Cc1ccc(O)c(NC(=O)C(C)NC(=O)OCc2ccccc2)c1)NC(=O)OC(C)(C)C)C(=O)OC(C)(C)C ; CC(N)C(=O)Nc1cc(C[C@@H](CC(C)C(=O)OC(C)(C)C)NC(=O)OC(C)(C)C)ccc1O]

In a hydrogenation bottle, Pd/C (0.1 g, 33 wt %, 50% wet) was added to a solution of (4R)-tert-butyl-5-(3-(2-(((benzyloxy)carbonyl)amino)propanamido)-4-hydroxyphenyl)-4-((tert-butoxycarbonyl)amino)-2-methylpentanoate (0.3 g, 0.5 mmol) in MeOH (10 mL). The mixture was shaken overnight under 1 atm H₂ and then filtered through Celite (filter aid). The filtrate was concentrated to afford the title compound (0.24 g, 100%) which was used for next step without further purification. ESI: m/z: calcd for C₂₄H₄₀N₃O₆ [M+H]⁺:466.28. found 466.28.

### Example 178. Synthesis of (4R)-tert-butyl-5-(3-(2-(2-(((benzyloxy)carbonyl)amino)-propanamido)propanamido)-4-hydroxyphenyl)-4-((tert-butoxycarbonyl)amino)-2-methylpentanoate

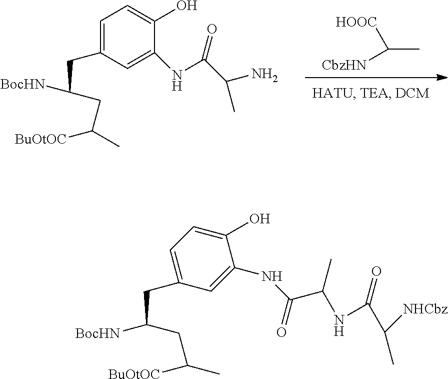

(4R)-Tert-butyl-5-(3-(2-aminopropanamido)-4-hydroxyphenyl)-4-((tert-butoxy-carbonyl)amino)-2-methylpentanoate (0.24 g, 0.5 mmol), 2-(((benzyloxy)carbonyl)amino)-propanoic acid (0.11 g, 0.5 mmol) and HATU (0.2 g, 0.5 mmol) were dissolved in DCM (20 ml), followed by addition of TEA (110 ul, 0.8 mmol). The reaction mixture was stirred at RT overnight, concentrated under reduced pressure and purified on SiO₂ column to give the title product (0.28 g, 85%). ESI: m/z: calcd for C₃₅H₅₁N₄O₉ [M+H]⁺: 671.36. found 671.35.

### Example 179. Synthesis of (4R)-tert-butyl-5-(3-(2-(2-aminopropanamido)propanamido)-4-hydroxyphenyl)-4-((tert-butoxycarbonyl)amino)-2-methylpentanoate

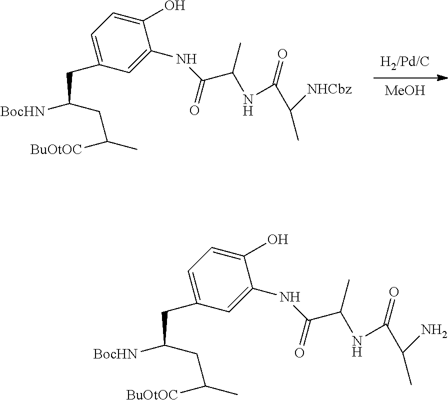

In a hydrogenation bottle, Pd/C (0.028 g, 10 wt %, 50% wet) was added to a solution of (4R)-tert-butyl-5-(3-(2-(2-(((benzyloxy)carbonyl)amino)propanamido)propanamido)-4-hydroxyphenyl)-4-((tert-butoxycarbonyl)amino)-2-methylpentanoate (0.28 g, 0.42 mmol) in MeOH (10 mL). The mixture was shaken overnight under 1 atm H₂ and then filtered through Celite (filter aid). The filtrate was concentrated to afford the title compound (0.18 g, 100%) which was used for next step without further purification. ESI: m/z: calcd for C₂₇H₄₅N₄O₇ [M+H]⁺:437.32. found 437.31.

### Example 180. Synthesis of B-10 (a Tubulysin Fragment Having a Bis-Linker)

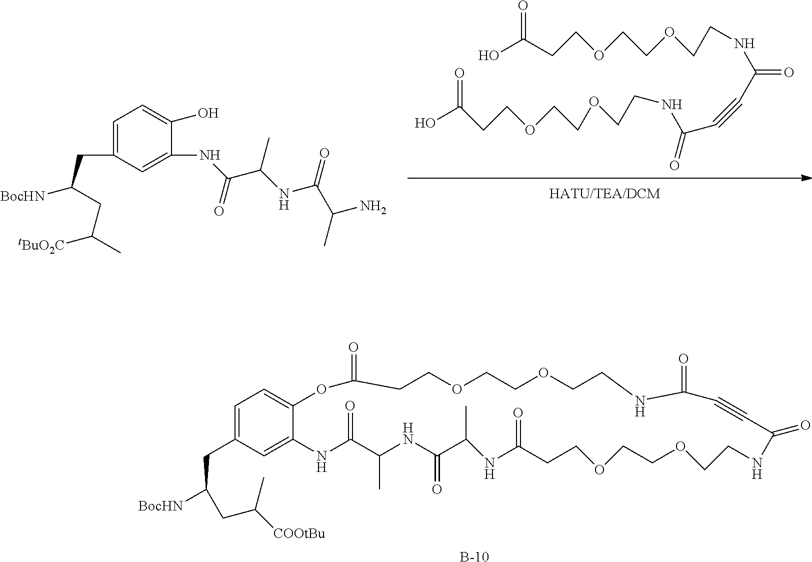

(4R)-tert-butyl-5-(3-(2-(2-aminopropanamido)propanamido)-4-hydroxyphenyl)-4-((tert-butoxycarbonyl)amino)-2-methylpentanoate (0.064 g, 0.12 mmol), 11,14-dioxo-4,7,18,21-tetraoxa-10,15-diazatetracos-12-yne-1,24-dioic acid (0.042 g, 0.097 mmol) and HATU (0.073 g, 0.194 mmol) were dissolved in DCM (10 ml), followed by addition of TEA (27.5 ul, 0.2 mmol). The reaction mixture was stirred at RT overnight, concentrated under reduced pressure and purified on SiO₂ column to give the title product B-10 (0.074 g, 82%). ESI: m/z: calcd for C₄₅H₆₉N₆O₁₅ [M+H]⁺: 933.47. found 933.46.

### Example 181. Synthesis of B-11 (a Tubulysin Fragment Having a Bis-Linker)

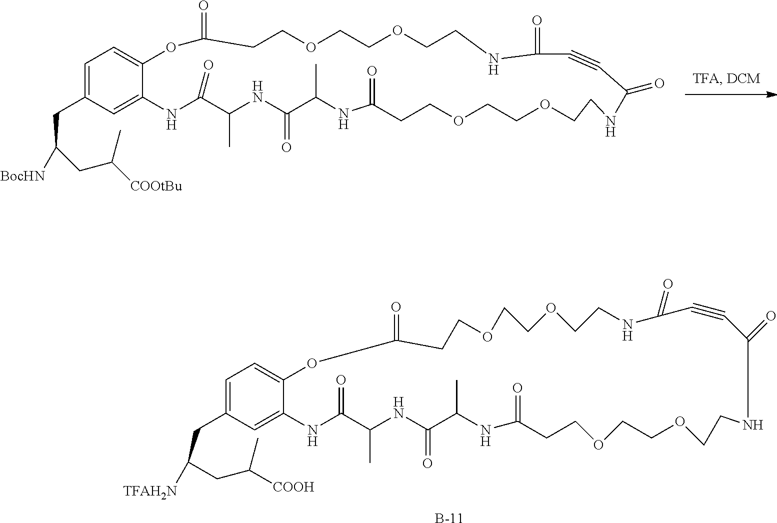

Compound B-10 (0.074 g, 0.08 mmol) was dissolved in DCM (1 ml), followed by addition of TFA (1 ml). The reaction was stirred at RT for 2h, concentrated to afford compound B-11 which was used for next step without further purification.

### Example 182. Synthesis of B-12 (a Tubulysin Analog Having a Bis-Linker)

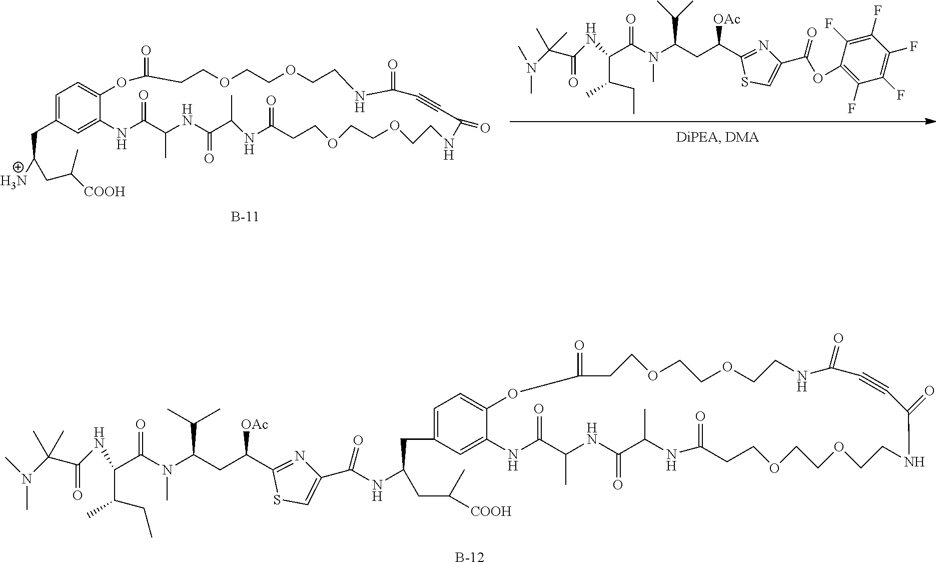

To the solution of compound B-11 (62.08 mg, 0.08 mmol) in DMA (1 ml) was added pentafluo-actived acid compound (55.36 mg, 0.08 mmol), then DIPEA (27 ul, 0.16 mmol) was added, the reaction was stirred overnight. Then the solution was concentrated and purified by HPLC with a gradient of MeCN/H₂O (10% MeCN to 70% MeCN in 45 min, C-18 column, 10 mm (d)×250 mm (1), 9 ml/min) to give the title product B-12 (20 mg, 20%). ESI: m/z: calcd for C₆₀H₉₁N₁₀O₁₈S [M+H]⁺: 1285.63. found 1285.63.

### Example 183. Synthesis of B-13 (a Tubulysin Fragment Having a Bis-Linker)

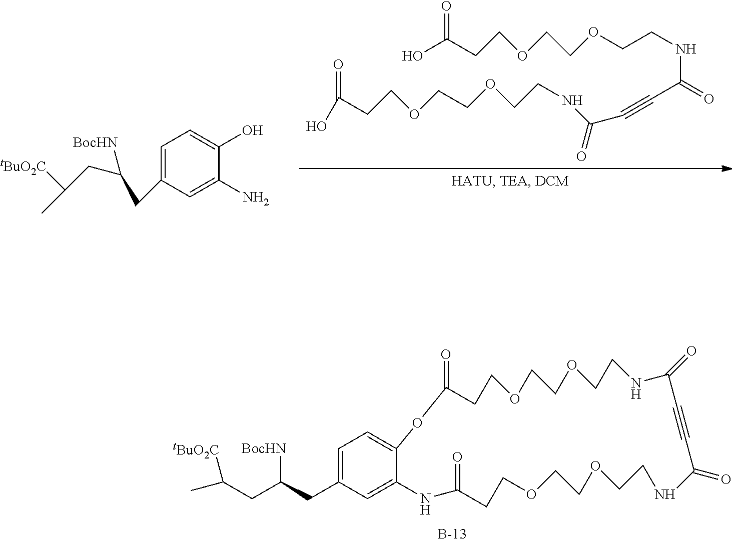

(4R)-tert-butyl-5-(3-amino-4-hydroxyphenyl)-4-((tert-butoxycarbonyl)amino)-2-methylpentanoate (0.19 g, 0.48 mmol), 11,14-dioxo-4,7,18,21-tetraoxa-10,15-diazatetracos-12-yne-1,24-dioic acid (0.173 g, 0.4 mmol) and HATU (0.3 g, 0.8 mmol) were dissolved in DCM (50 ml), followed by addition of TEA (110 ul, 0.8 mmol). The reaction mixture was stirred at RT overnight, concentrated under reduced pressure and purified on SiO₂ column to give the title product B-13 (0.25 g, 80%). ESI: m/z: calcd for C₃₉H₅₉N₄O₁₃ [M+H]⁺: 791.40. found 791.40.

### Example 184. Synthesis of B-14 (a Tubulysin Fragment Having a Bis-Linker)

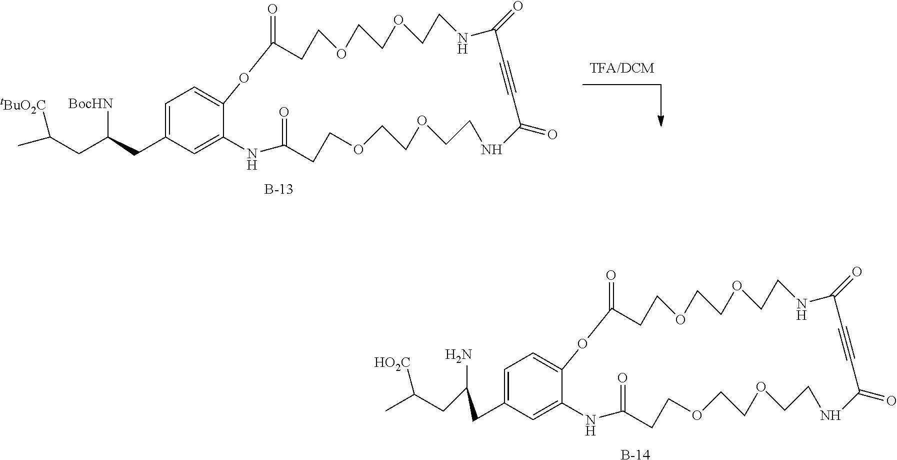

Compound B-13 (0.1 g, 0.14 mmol) was dissolved in DCM (1 ml), followed by addition of TFA (0.8 ml). The reaction mixture was stirred at RT for 2h and then concentrated to afford compound B-14 which was used for next step without further purification.

### Example 185. Synthesis of B-15 (a Tubulysin Analog Having a Bis-Linker)

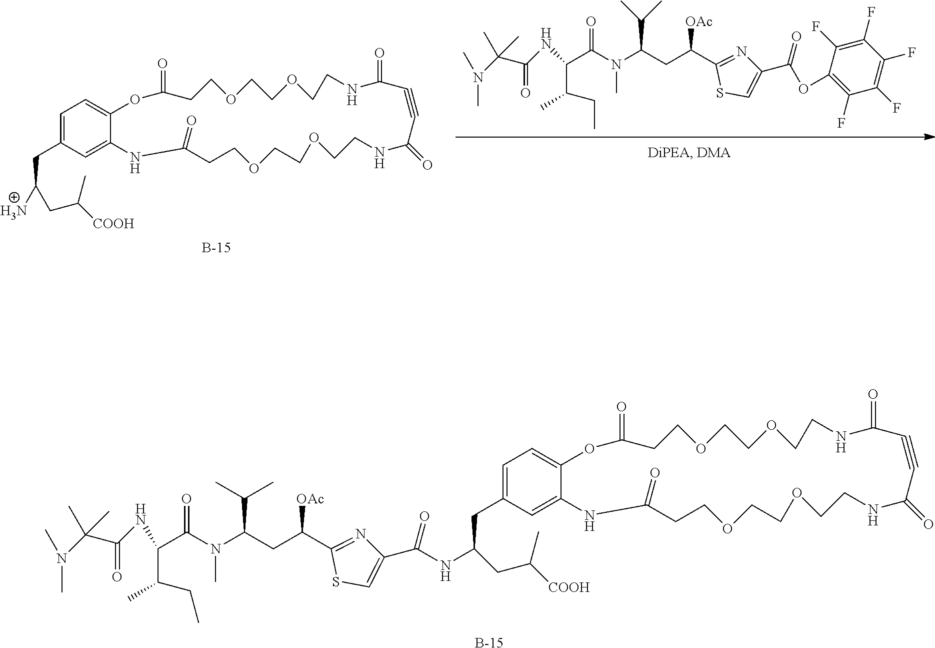

To the solution of compound B-14 (88.76 mg, 0.14 mmol) in DMA (1 ml) was added pentafluo-actived acid compound (96.88 mg, 0.14 mmol), then DIPEA (47.5 ul, 0.28 mmol) was added, the reaction was stirred overnight. Then the solution was concentrated and purified by HPLC with a gradient of MeCN/H₂O (10% MeCN to 70% MeCN in 45 min, C-18 column, 10 mm (d)×250 mm (1), 9 ml/min) to give the title product B-15 (40 mg, 25%). ESI: m/z: calcd for C₅₅H₈₃N₈O₁₆S [M+H]⁺: 1143.56. found 1143.56.

### Example 186. Synthesis of (4R)-tert-butyl-5-(3-(4-(((benzyloxy)carbonyl)amino)-butanamido)-4-hydroxyphenyl)-4-((tert-butoxycarbonyl)amino)-2-methylpentanoate

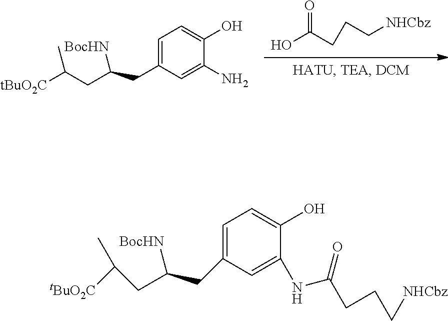

(4R)-tert-butyl-5-(3-amino-4-hydroxyphenyl)-4-((tert-butoxycarbonyl)amino)-2-methylpentanoate (0.2 g, 0.5 mmol), 4-(((benzyloxy)carbonyl)amino)butanoic acid (0.12 g, 0.5 mmol) and HATU (0.2 g, 0.5 mmol) were dissolved in DCM (50 ml), followed by addition of TEA (110 ul, 0.8 mmol). The reaction mixture was stirred at RT overnight, concentrated under reduced pressure and purified on SiO₂ column to give the title product (0.26 g, 85%). ESI: m/z: calcd for C₃₃H₄₈N₃O₈ [M+H]⁺: 614.34. found 614.34.

### Example 187. Synthesis of (4R)-tert-butyl-5-(3-(4-aminobutanamido)-4-hydroxyphenyl)-4-((tert-butoxycarbonyl)amino)-2-methylpentanoate

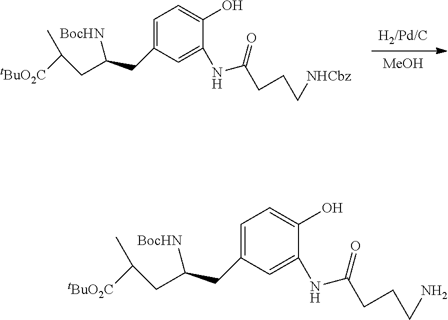 [SMILES: "MeOH" ; CC(C[C@H](Cc1ccc(O)c(NC(=O)CCCNC(=O)OCc2ccccc2)c1)NC(=O)OC(C)(C)C)C(=O)OC(C)(C)C ; "H2/Pd/C" ; CC(C[C@H](Cc1ccc(O)c(NC(=O)CCCN)c1)NC(=O)OC(C)(C)C)C(=O)OC(C)(C)C]

In a hydrogenation bottle, Pd/C (0.028 g, 10 wt %, 50% wet) was added to a solution of (4R)-tert-butyl-5-(3-(4-(((benzyloxy)carbonyl)amino)butanamido)-4-hydroxyphenyl)-4-((tert-butoxycarbonyl)amino)-2-methylpentanoate (0.09 g, 0.15 mmol) in MeOH (10 mL). The mixture was shaken overnight under 1 atm H₂ and then filtered through Celite (filter aid). The filtrate was concentrated to afford the title compound (0.07 g, 100%) which was used for the next step without further purification. ESI: m/z: calcd for C₂₅H₄₂N₃O₆[M+H]⁺: 480.30. found 480.31.

### Example 188. Synthesis of B-16 (a Tubulysin Fragment Having a Bis-Linker)

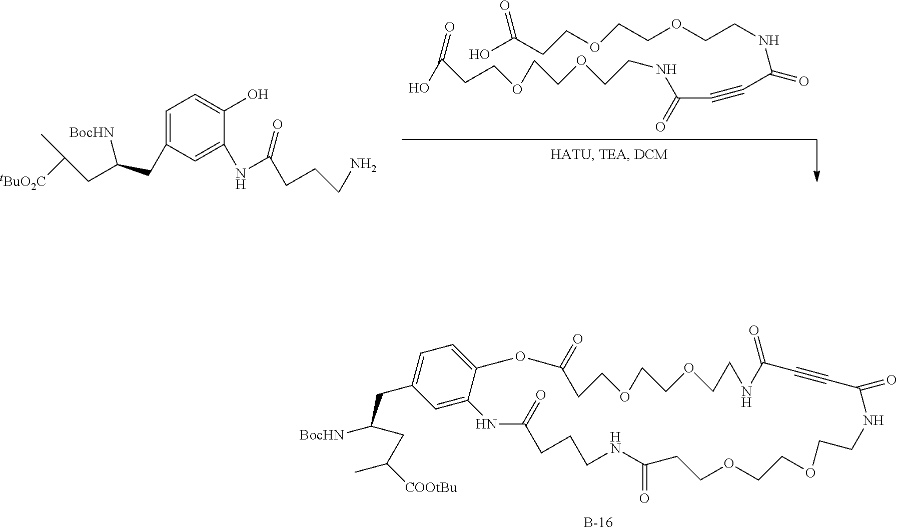

(4R)-tert-butyl-5-(3-(4-aminobutanamido)-4-hydroxyphenyl)-4-((tert-butoxycarbonyl)-amino)-2-methylpentanoate (39 mg, 0.08 mmol), 11,14-dioxo-4,7,18,21-tetraoxa-10,15-diazatetracos-12-yne-1,24-dioic acid (43 mg, 0.1 mmol) and HATU (30.4 mg, 0.08 mmol) were dissolved in DCM (20 ml), followed by addition of TEA (22 ul, 0.16 mmol). The reaction mixture was stirred at RT overnight, concentrated under reduced pressure and purified on SiO₂ column to give the title product B-16 (42 mg, 60%). ESI: m/z: calcd for C₄₃H₆₆N₅O₁₄ [M+H]⁺: 876.45. found 876.40.

### Example 189. Synthesis of B-17 (a Tubulysin Fragment Having a Bis-Linker)

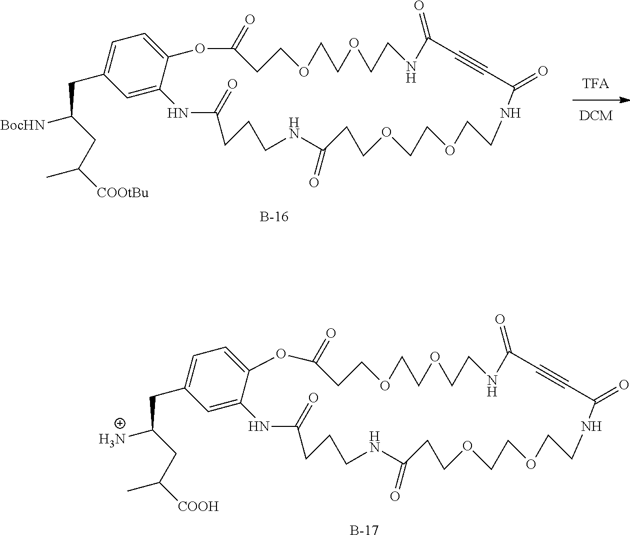

Compound B-16 (17 mg, 0.019 mmol) was dissolved in DCM (0.8 ml), followed by addition of TFA (0.5 ml). The reaction mixture was stirred at RT for 2h and then concentrated to afford compound B-17 (17 mg, >100%) which was used for the next step without further purification. ESI: m/z: calcd for C₃₄H₅₀N₅O₁₂ [M+H]⁺: 720.34. found 720.70.

### Example 190. Synthesis of B-18 (a Tubulysin Analog Having a Bis-Linker)

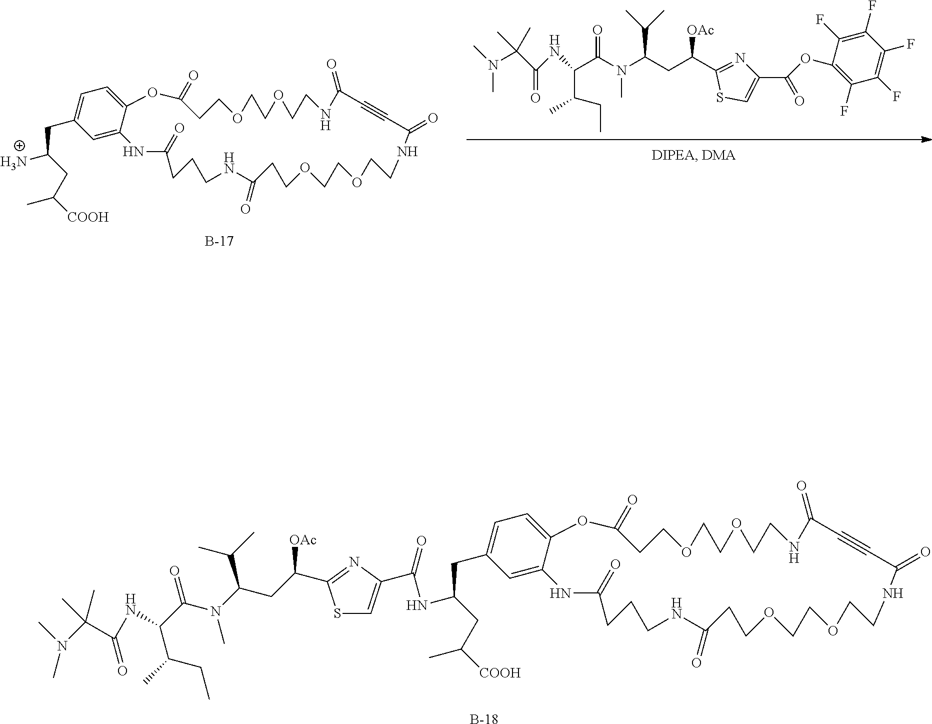

To the solution of compound B-17 (13.6 mg, 0.019 mmol) in DMA (1 ml) was added pentafluo-actived acid compound (13 mg, 0.019 mmol) and DIPEA (6.4 ul, 0.038 mmol). The reaction mixture was stirred overnight, concentrated and purified on HPLC with a gradient of MeCN/H₂O (10% MeCN to 70% MeCN in 45 min, C-18 column, 10 mm (d)×250 mm (1), 9 ml/min) to give the title product B-18 (9.9 mg, 42%). ESI: m/z: calcd for C₅₉H₉₀N90₁₇S [M+H]⁺: 1228.61. found 1228.60.

### Example 191. Synthesis of (4R)-tert-butyl-4-((tert-butoxycarbonyl)amino)-5-(3-(4-(2,5-dioxo-2,5-dihydro-1H-pyrrol-1-yl)butanamido)-4-((4-(2,5-dioxo-2,5-dihydro-1H-pyrrol-1-yl)butanoyl)oxy)phenyl)-2-methylpentanoate

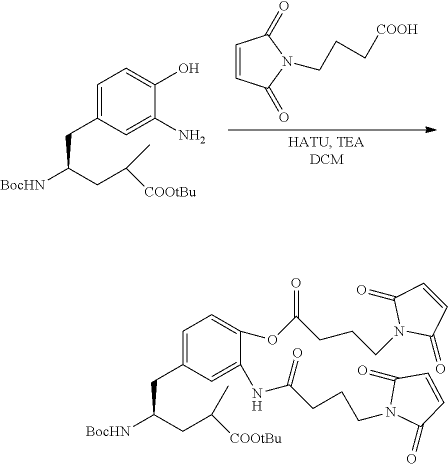

(4R)-tert-butyl-5-(3-amino-4-hydroxyphenyl)-4-((tert-butoxycarbonyl)amino)-2-methylpentanoate (68 mg, 0.17 mmol), 4-(2,5-dioxo-2,5-dihydro-1H-pyrrol-1-yl)butanoic acid (94.5 mg, 0.52 mmol) and HATU (161.5 mg, 0.425 mmol) were dissolved in DCM (50 ml), followed by addition of TEA (73 ul, 0.52 mmol). The reaction mixture was stirred at RT overnight, concentrated under reduced pressure and purified by SiO₂ column eluted with EtOAc/DCM (1:10) to give the title product (98 mg, 80%). ESI: m/z: calcd for C₃₇H₄₉N₄O₁₁ [M+H]⁺: 725.33. found 725.34.

### Example 192. Synthesis of (2R)-4-carboxy-1-(3-(4-(2,5-dioxo-2,5-dihydro-1H-pyrrol-1-yl)butanamido)-4-((4-(2,5-dioxo-2,5-dihydro-1H-pyrrol-1-yl)butanoyl)oxy)phenyl)pentan-2-aminium, TFA Salt

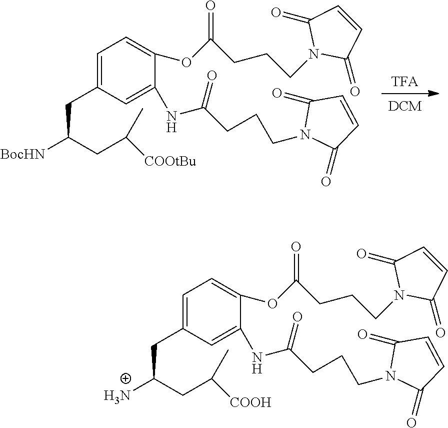

(4R)-tert-butyl-4-((tert-butoxycarbonyl)amino)-5-(3-(4-(2,5-dioxo-2,5-dihydro-1H-pyrrol-1-yl)butanamido)-4-((4-(2,5-dioxo-2,5-dihydro-1H-pyrrol-1-yl)butanoyl)oxy)phenyl)-2-methylpentanoate (98 mg, 0.135 mmol) was dissolved in DCM (5 ml), followed by addition of TFA (3 ml). The reaction mixture was stirred at RT for 2h and then concentrated to afford the title compound (95 mg, >100% yield) which was used for next step without further purification. ESI: m/z: calcd for C₂₈H₃₃N₄O₉ [M+H]⁺: 569.22. found 569.60.

### Example 193. Synthesis of (4R)-4-(2-((6S,9R,11R)-6-((S)-sec-butyl)-9-isopropyl-2,3,3,8-tetramethyl-4,7,13-trioxo-12-oxa-2,5,8-triazatetradecan-11-yl)thiazole-4-carboxamido)-5-(3-(4-(2,5-dioxo-2,5-dihydro-1H-pyrrol-1-yl)butanamido)-4-((4-(2,5-dioxo-2,5-dihydro-1H-pyrrol-1-yl)butanoyl)oxy)phenyl)-2-methylpentanoic Acid (B-19)

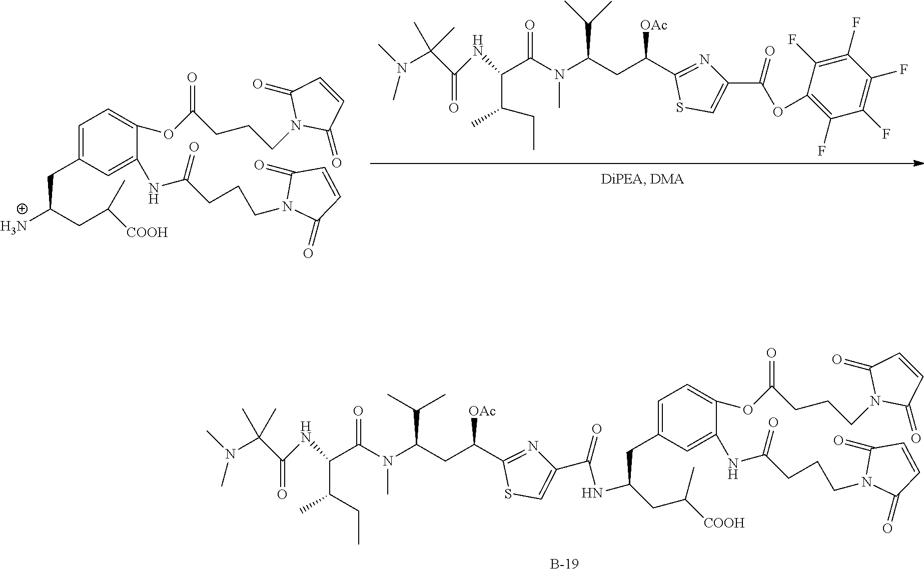

To the solution of (2R)-4-carboxy-1-(3-(4-(2,5-dioxo-2,5-dihydro-1H-pyrrol-1-yl)-butanamido)-4-((4-(2,5-dioxo-2,5-dihydro-1H-pyrrol-1-yl)butanoyl)oxy)phenyl)pentan-2-aminium, TFA salt (76.9 mg, 0.135 mmol) in DMA (1 ml) was added pentafluo-actived acid compound (44 mg, 0.06 mmol) and DIPEA (45.8 ul, 0.27 mmol). The reaction mixture was stirred overnight, concentrated and purified on HPLC with a gradient of MeCN/H₂O (10% MeCN to 70% MeCN in 45 min, C-18 column, 10 mm (d)×250 mm (1), 9 ml/min) to give the title product B-19 (37 mg, 55%). ESI: m/z: calcd for C₅₃H₇₃N₈O₁₄S [M+H]⁺: 1077.49. found 1077.50.

### Example 194. Synthesis of (4R)-tert-butyl 4-((tert-butoxycarbonyl)amino)-5-(3-(3-(2-(2-(2-(2,5-dioxo-2,5-dihydro-1H-pyrrol-1-yl)ethoxy)ethoxy)ethoxy)propanamido)-4-((3-(2-(2-(2-(2,5-dioxo-2,5-dihydro-1H-pyrrol-1-yl)ethoxy)ethoxy)ethoxy)propanoyl)oxy)phenyl)-2-methylpentanoate

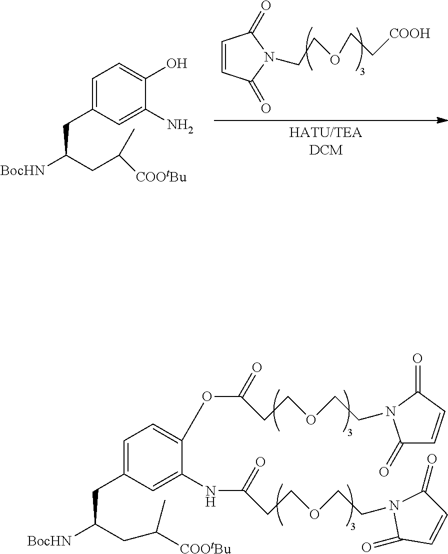

(4R)-tert-butyl-5-(3-amino-4-hydroxyphenyl)-4-((tert-butoxycarbonyl)amino)-2-methylpentanoate (100 mg, 0.25 mmol), 3-(2-(2-(2-(2,5-dioxo-2,5-dihydro-1H-pyrrol-1-yl)ethoxy)ethoxy)ethoxy)propanoic acid (75 mg, 0.25 mmol) and HATU (190 mg, 0.5 mmol) were dissolved in DCM (50 ml), followed by addition of TEA (73 ul, 0.5 mmol). The reaction mixture was stirred at RT overnight, concentrated under reduced pressure and purified on SiO₂ column eluted with EtOAc/DCM (1:3) to give the title product (180.05 mg, 75%). ESI: m/z: calcd for C₄₇H₆₉N₄O₁₇ [M+H]⁺: 961.45. found 961.81.

### Example 195. Synthesis of (2R)-4-carboxy-1-(3-(3-(2-(2-(2-(2,5-dioxo-2,5-dihydro-1H-pyrrol-1-yl)ethoxy)ethoxy)ethoxy)propanamido)-4-((3-(2-(2-(2-(2,5-dioxo-2,5-dihydro-1H-pyrrol-1-yl)ethoxy)ethoxy)ethoxy)propanoyl)oxy)phenyl)pentan-2-aminium, TFA Salt

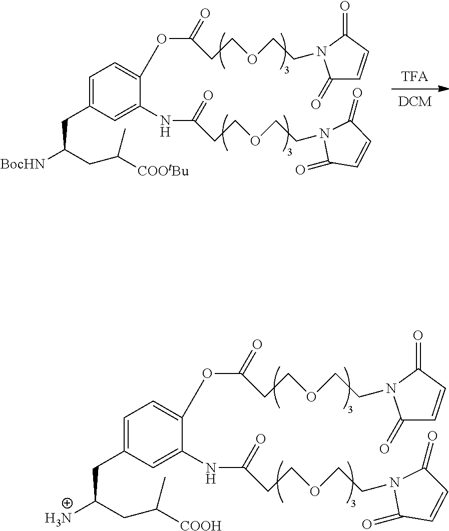

(4R)-Tert-butyl 4-((tert-butoxycarbonyl)amino)-5-(3-(3-(2-(2-(2-(2,5-dioxo-2,5-dihydro-1H-pyrrol-1-yl)ethoxy)ethoxy)ethoxy)propanamido)-4-((3-(2-(2-(2-(2,5-dioxo-2,5-dihydro-1H-pyrrol-1-yl)ethoxy)ethoxy)ethoxy)propanoyl)oxy)phenyl)-2-methylpentanoate (180.0 mg, 0.187 mmol) was dissolved in DCM (12 ml), followed by addition of TFA (6 ml). The reaction mixture was stirred at RT for 2h, then concentrated, and co-evaporated with DCM/toluene to dryness to afford the title compound (155 mg, >100% yield) which was used for next step without further purification. ESI: m/z: calcd for C₃₈H₅₄N₄O₁₅ [M+H]⁺: 805.35. found 805.60.

### Example 196. Synthesis of (4R)-4-(2-((6S,9R,11R)-6-((S)-sec-butyl)-9-isopropyl-2,3,3,8-tetramethyl-4,7,13-trioxo-12-oxa-2,5,8-triazatetradecan-11-yl)thiazole-4-carboxamido)-5-(3-(3-(2-(2-(2-(2,5-dioxo-2,5-dihydro-1H-pyrrol-1-yl)ethoxy)ethoxy)ethoxy)propanamido)-4-((3-(2-(2-(2-(2,5-dioxo-2,5-dihydro-1H-pyrrol-1-yl)ethoxy)ethoxy)ethoxy)propanoyl)oxy)phenyl)-2-methylpentanoic Acid (B-20)

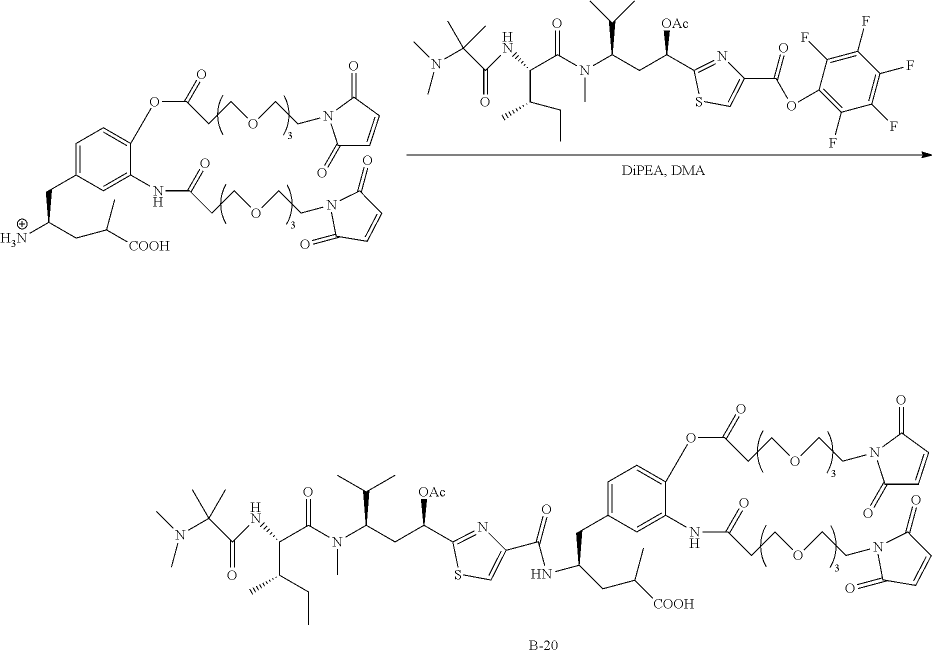

To the solution of (2R)-4-carboxy-1-(3-(3-(2-(2-(2-(2,5-dioxo-2,5-dihydro-1H-pyrrol-1-yl)ethoxy)ethoxy)ethoxy)propanamido)-4-((3-(2-(2-(2-(2,5-dioxo-2,5-dihydro-1H-pyrrol-1-yl)ethoxy)ethoxy)ethoxy)propanoyl)oxy)phenyl)pentan-2-aminium, TFA salt (43 mg, 0.06 mmol) in DMA (1 ml) was added pentafluo-actived acid compound (48.5 mg, 0.06 mmol) and DIPEA (34 ul, 0.2 mmol). The reaction mixture was stirred overnight, concentrated and purified on HPLC with a gradient of MeCN/H₂O (10% MeCN to 70% MeCN in 45 min, C-18 column, 10 mm (d)×250 mm (1), 9 ml/min) to give the title product B-20 (35 mg, 45%). ESI: m/z: calcd for C₅₉H₈₅N₈O₁₈S [M+H]⁺: 1313.61. found 1313.85.

### Example 197. Synthesis of (4R)-5-(22,23-bis(2,5-dioxo-2,5-dihydro-1H-pyrrol-1-yl)-3,6,39,42-tetramethyl-2,5,8,21,24,37,40,43-octaoxo-3,4,5,6,7,8,9,10,12,13,15,16,18,19,20,21,22,23,24,25,26,27,29,30,32,33,35,36,37,38,39,40,41,42,43,44-hexatriacontahydro-2H-benzo[b][1,14,17,20,31,34,37,4,7,10,23,28,41,44]heptaoxaheptaazacyclohexatetracontin-46-yl)-4-(2-((6S,9R,11R)-6-((S)-sec-butyl)-9-isopropyl-2,3,3,8-tetramethyl-4,7,13-trioxo-12-oxa-2,5,8-triazatetradecan-11-yl)thiazole-4-carboxamido)-2-methylpentanoic Acid (B-21)

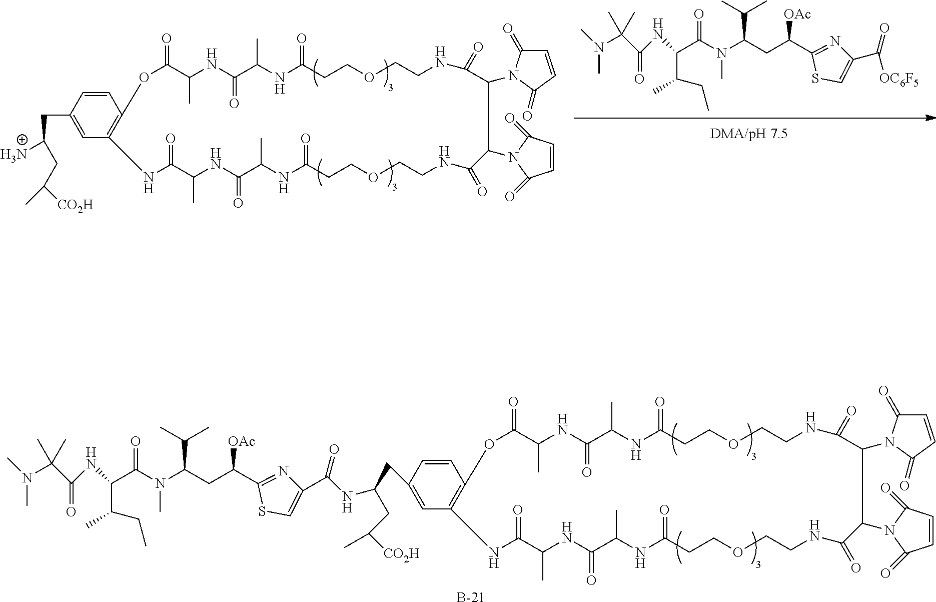

To the solution of (2R)-1-(22,23-bis(2,5-dioxo-2,5-dihydro-1H-pyrrol-1-yl)-3,6,39,42-tetramethyl-2,5,8,21,24,37,40,43-octaoxo-3,4,5,6,7,8,9,10,12,13,15,16,18,19,20,21,22,23,24,25,26,27,29,30,32,33,35,36,37,38,39,40,41,42,43,44-hexatriacontahydro-2H-benzo[b][1,14,17,20,31,34,37,4,7,10,23,28,41,44]heptaoxaheptaazacyclohexatetracontin-46-yl)-4-carboxypentan-2-aminium TFA salt (60 mg, 0.050 mmol) in DMA (1.5 ml) was added pentafluo-actived acid compound (44 mg, 0.06 mmol) and 0.1 M NaH₂PO₄, pH 7.5, 0.8 ml. The reaction mixture was stirred overnight, concentrated and purified on HPLC with a gradient of MeCN/H₂O (10% MeCN to 70% MeCN in 45 min, C-18 column, 10 mm (d)×250 mm (1), 8 ml/min) to give the title product B-21 (44 mg, 52% yield). ESI: m/z: calcd for C₇₉H₁₁₇N₁₄O₂₆S [M+H]⁺: 1709.79, found 1709.55.

### Example 198. Synthesis of (4R)-4-(2-((4R,6R,9S,12S,15 S,18S)-9-((S)-sec-butyl)-6,12-diisopropyl-7,13,15,18-tetramethyl-2,8,11,14,17,20,23-heptaoxo-21-propiolamido-3-oxa-7,10,13,16,19,22-hexaazapentacos-24-yn-4-yl)thiazole-4-carboxamido)-2-methyl-5-phenylpentanoic Acid (B-22)

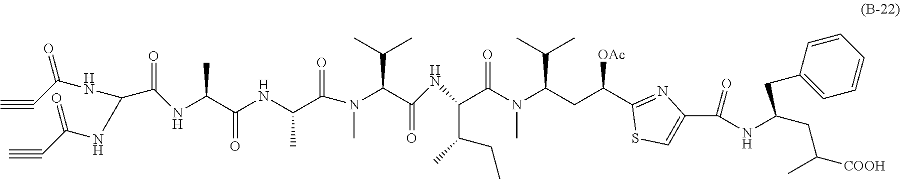 [SMILES: C#CC(=O)NC(NC(=O)C#C)C(=O)N[C@@H](C)C(=O)N[C@@H](C)C(=O)N(C)[C@H](C(=O)N[C@H](C(=O)N(C)[C@H](C[C@@H](OC(C)=O)c1nc(C(=O)N[C@@H](Cc2ccccc2)CC(C)C(=O)O)cs1)C(C)C)[C@@H](C)CC)C(C)C]

To (4R)-4-(2-((3S,6S,9R,11R)-6-((S)-sec-butyl)-3,9-diisopropyl-8-methyl-4,7,13-trioxo-12-oxa-2,5,8-triazatetradecan-11-yl)thiazole-4-carboxamido)-2-methyl-5-phenylpentanoic acid hydrochloride salt (25 mg, 0.034 mmol) in the mixture of DMA (2 ml) and 0.1 M Na₂HPO₄, pH 8.0 (1 ml) was added (S)-2,5-dioxopyrrolidin-1-yl 2-((S)-2-(2,2-dipropiolamido-acetamido)propanamido)propanoate (23.1 mg, 0.053 mmol) in three portions in 3 h and the mixture was then stirred for another 12 hr. The mixture was concentrated, and purified by reverse phase HPLC (200 (L) mm×10(d) mm, C₁₈ column, 10-100% acetonitrile/water in 40 min, v=8 ml/min) to afford the title compound (30.0 mg, 85% yield). ESI MS m/z: calcd for C₅₁H₇₁N₉O₁₂S [M+H]⁺ 1034.49. found 1034.90.

### Example 199. Synthesis of (4R)-4-(2-((1R,3R)-1-acetoxy-3-((2S,3S)—N,3-dimethyl-2-((R)-1-methylpiperidine-2-carboxamido)pentanamido)-4-methylpentyl)thiazole-4-carboxamido)-5-(4-hydroxy-3-(3-(2-(2-((bis((Z)-3-carboxyacrylhydrazinyl)phosphoryl)amino)ethoxy)ethoxy)-propanamido)phenyl)-2-methylpentanoic Acid (B-23)

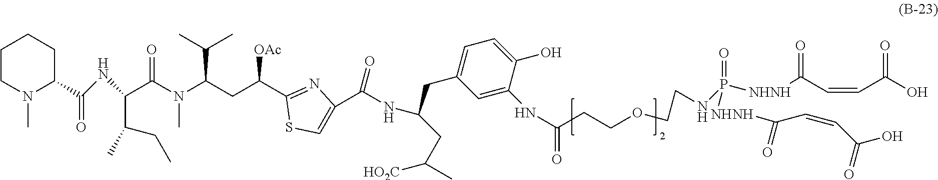

To compound (Z)-3-carboxyacrylhydrazide HCl salt (22.0 mg, 0.132 mmol) in the mixture of THF (5 ml) and DIPEA (10 μl, 0.057 mmol) at 0° C. was added POCl₃ (10.1 mg, 0.0665 mmol). After stirred at 0° C. for 20 min, the mixture was warmed to room temperature and kept to stirring for another 4 h. Then to the mixture was added compound (4R)-4-(2-((1R,3R)-1-acetoxy-3-((2 S,3S)—N,3-dimethyl-2-((R)-1-methylpiperidine-2-carboxamido)pentanamido)-4-methylpentyl)thiazole-4-carboxamido)-5-(3-(3-(2-(2-aminoethoxy)ethoxy)propanamido)-4-hydroxyphenyl)-2-methylpentanoic acid (60 mg, 0.065 mmol) and DIPEA (20 μl, 0.114 mmol). The mixture was stirred at 50° C. for overnight, concentrated, and purified by reverse phase HPLC (250 (L) mm×10(d) mm, C₁g column, 10-100% acetonitrile/water in 40 min, v=8 ml/min) to afford the title compound (23.1 mg, 32% yield). ESI MS m/z: calcd for C₅₃H₈₁N₁₁O₁₁PS [M+H]⁺ 1222.51. found 1222.80.

### Example 200. Synthesis of (1R,3R)-1-(4-(((2R)-5-((2-aminoethyl)amino)-1-(22,23-bis(2,5-dioxo-2,5-dihydro-1H-pyrrol-1-yl)-3,6,39,42-tetramethyl-2,5,8,21,24,37,40,43-octaoxo-3,4,5,6,7,8,9,10,12,13,15,16,18,19,20,21,22,23,24,25,26,27,29,30,32,33,35,36,37,38,39,40,41,42,43,44-hexatriacontahydro-2H-benzo[b][1,14,17,20,31,34,37,4,7,10,23,28,41,44]heptaoxaheptaaza-cyclohexatetracontin-46-yl)-4-methyl-5-oxopentan-2-yl)carbamoyl)thiazol-2-yl)-3-((2S,3S)-2-(2-(dimethylamino)-2-methylpropanamido)-N,3-dimethylpentanamido)-4-methylpentyl Acetate (B-24)

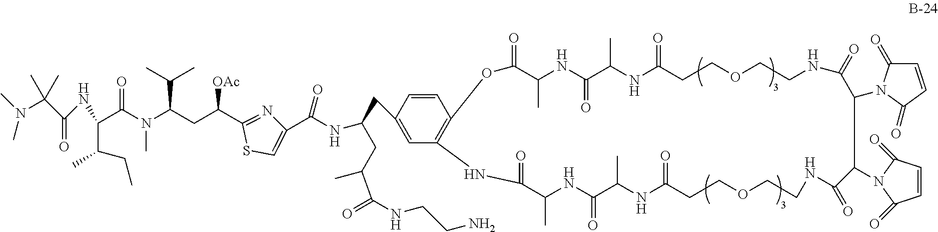

Compound B-21 (22.0 mg, 0.0129 mmol) in DMA (1 ml) was added EDC (15.0 mg, 0.078 mmol), ethane-1,2-diamine hydrochloride salt (8.0 mg, 0.060 mmol) and DIPEA (0.010 ml, 0.060 mmol). The mixture was stirred for overnight, concentrated, and purified by reverse phase HPLC (250 (L) mm×10(d) mm, C₁₈ column, 10-100% acetonitrile/water in 40 min, v=8 ml/min) to afford the title compound (14.0 mg, 62% yield). ESI MS m/z: calcd for C₈₁H₁₂₃N₁₆O₂₅S [M+H]⁺ 1751.85. found 1751.20.

### Example 201. Synthesis of (1R,3R)-1-(4-(((28R)-1-amino-29-(22,23-bis(2,5-dioxo-2,5-dihydro-1H-pyrrol-1-yl)-3,6,39,42-tetramethyl-2,5,8,21,24,37,40,43-octaoxo-3,4,5,6,7,8,9,10,12,13,15,16,18,19,20,21,22,23,24,25,26,27,29,30,32,33,35,36,37,38,39,40,41,42,43,44-hexatriacontahydro-2H-benzo[b][1,14,17,20,31,34,37,4,7,10,23,28,41,44]heptaoxaheptaaza-cyclohexatetracontin-46-yl)-26-methyl-25-oxo-3,6,9,12,15,18,21-heptaoxa-24-azanonacosan-28-yl)carbamoyl)thiazol-2-yl)-3-((2S,3S)-2-(2-(dimethylamino)-2-methylpropanamido)-N,3-dimethylpentanamido)-4-methylpentyl Acetate (B-25)

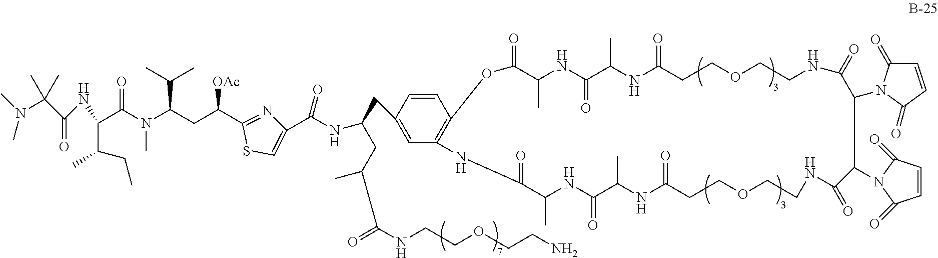

Compound B-21 (22.0 mg, 0.0129 mmol) in DMA (1 ml) was added EDC (15.0 mg, 0.078 mmol), 3,6,9,12,15,18,21-heptaoxatricosane-1,23-diamine hydrochloride salt (26.0 mg, 0.059 mmol) and DIPEA (0.010 ml, 0.060 mmol). The mixture was stirred for overnight, concentrated, and purified by reverse phase HPLC (250 (L) mm×10(d) mm, C₁₈ column, 10-100% acetonitrile/water in 40 min, v=8 ml/min) to afford the title compound (14.5 mg, 55% yield). ESI MS m/z: calcd for C₉₅H₁₅₁N₁₆O₃₂S [M+H]⁺ 2060.03. found 2060.80.

### Example 202. Synthesis of (1R,3R)-1-(4-(((28R)-29-(22,23-bis(2,5-dioxo-2,5-dihydro-1H-pyrrol-1-yl)-3,6,39,42-tetramethyl-2,5,8,21,24,37,40,43-octaoxo-3,4,5,6,7,8,9,10,12,13,15,16,18,19,20,21,22,23,24,25,26,27,29,30,32,33,35,36,37,38,39,40,41,42,43,44-hexatriacontahydro-2H-benzo[b][1,14,17,20,31,34,37,4,7,10,23,28,41,44]heptaoxaheptaaza-cyclohexatetracontin-46-yl)-1-hydroxy-26-methyl-25-oxo-3,6,9,12,15,18,21-heptaoxa-24-azanonacosan-28-yl)carbamoyl)thiazol-2-yl)-3-((2S,3S)-2-(2-(dimethylamino)-2-methylpropanamido)-N,3-dimethylpentanamido)-4-methylpentyl acetate (B-26)

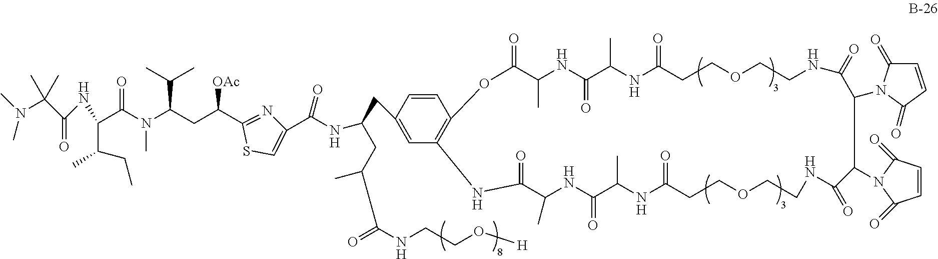

Compound B-21 (22.0 mg, 0.0129 mmol) in DMA (1 ml) was added EDC (15.0 mg, 0.078 mmol) and 23-amino-3,6,9,12,15,18,21-heptaoxatricosan-1-ol (22.0 mg, 0.059 mmol). The mixture was stirred for overnight, concentrated, and purified by reverse phase HPLC (250 (L) mm×10(d) mm, C₁₈ column, 10-100% acetonitrile/water in 40 min, v=8 ml/min) to afford the title compound (14.1 mg, 53% yield). ESI MS m/z: calcd for C₉₅H₁₅₀N₁₅O₃₃S [M+H]⁺2061.02. found 2061.74.

### Example 203. Synthesis of (2S)-tert-butyl 2-((4R)-5-(22,23-bis(2,5-dioxo-2,5-dihydro-1H-pyrrol-1-yl)-3,6,39,42-tetramethyl-2,5,8,21,24,37,40,43-octaoxo-3,4,5,6,7,8,9,10,12,13,15,16,18,19,20,21,22,23,24,25,26,27,29,30,32,33,35,36,37,38,39,40,41,42,43,44-hexatriacontahydro-2H-benzo[b][1,14,17,20,31,34,37,4,7,10,23,28,41,44]heptaoxahepta-azacyclohexatetracontin-46-yl)-4-(2-((6S,9R,11R)-6-((S)-sec-butyl)-9-isopropyl-2,3,3,8-tetramethyl-4,7,13-trioxo-12-oxa-2,5,8-triazatetradecan-11-yl)thiazole-4-carboxamido)-2-methylpentanamido)-6-((tert-butoxycarbonyl)amino)hexanoate (B-27)

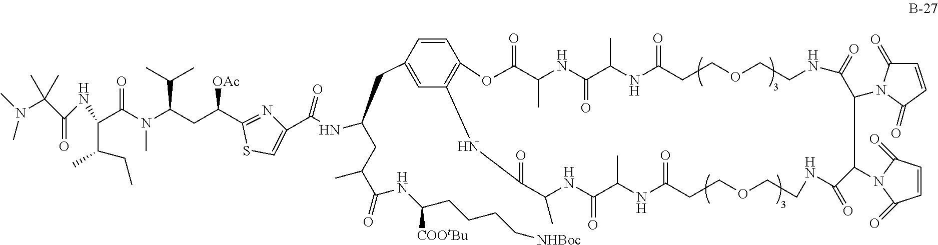

Compound B-21 (25.0 mg, 0.0146 mmol) in DMA (1 ml) was added EDC (15.0 mg, 0.078 mmol) and (S)-tert-butyl 2-amino-6-((tert-butoxycarbonyl)amino)hexanoate (9.0 mg, 0.030 mmol). The mixture was stirred for overnight, concentrated, and purified by reverse phase HPLC (250 (L) mm×10(d) mm, C₁₈ column, 10-100% acetonitrile/water in 40 min, v=8 ml/min) to afford the title compound (20.5 mg, 71% yield). ESI MS m/z: calcd for C₉₄H₁₄₄N₁₆O₂₉S [M+H]⁺ 1994.00. found 1994.85.

### Example 204. Synthesis of (2S)-6-amino-2-((4R)-5-(22,23-bis(2,5-dioxo-2,5-dihydro-1H-pyrrol-1-yl)-3,6,39,42-tetramethyl-2,5,8,21,24,37,40,43-octaoxo-3,4,5,6,7,8,9,10,12,13,15,16,18,19,20,21,22,23,24,25,26,27,29,30,32,33,35,36,37,38,39,40,41,42,43,44-hexatriacontahydro-2H-benzo[b][1,14,17,20,31,34,37,4,7,10,23,28,41,44]heptaoxaheptaaza-cyclohexatetracontin-46-yl)-4-(2-((6S,9R,11R)-6-((S)-sec-butyl)-9-isopropyl-2,3,3,8-tetramethyl-4,7,13-trioxo-12-oxa-2,5,8-triazatetradecan-11-yl)thiazole-4-carboxamido)-2-methylpentanamido)hexanoic Acid (B-28)

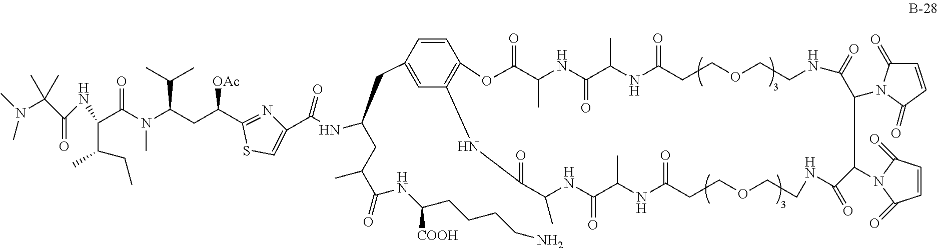

Compound B-27 (20.0 mg, 0.010 mmol) was dissolved in DCM (1 ml), followed by addition of TFA (1 ml). The reaction mixture was stirred at RT for 2h, then concentrated, and purified by reverse phase HPLC (250 (L) mm×10(d) mm, C₁₈ column, 10-100% acetonitrile/water in 40 min, v=8 ml/min) to afford the title compound (13.5 mg, 73% yield). ESI: m/z: calcd for C₈₅H₁₂₉N₁₆O₂₇S [M+H]⁺: 1837.89. found 1838.20.

### Example 205. Synthesis of (2S,4R)-methyl 4-hydroxypyrrolidine-2-carboxylate Hydrochloric

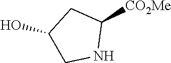

To a solution of trans-4-hydroxy-L-proline (15.0 g, 114.3 mmol) in dry methanol (250 mL) was added thionyl chloride (17 mL, 231 mmol) dropwise at 0 to 4° C. The resulting mixture was stirred for at r.t. overnight, concentrated, crystallized with EtOH/hexane to provide the title compound (18.0 g, 87% yield). ESI MS m/z 168.2 ([M+Na]⁺).

### Example 206. Synthesis of (2S,4R)-1-tert-butyl 2-methyl 4-hydroxypyrrolidine-1,2-dicarboxylate

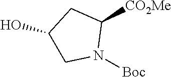

To a solution of trans-4-hydroxy-L-proline methyl ester (18.0 g, 107.0 mmol) in the mixture of MeOH (150 ml) and sodium bicarbonate solution (2.0 M, 350 ml) was added Boc₂O (30.0 g, 137.6 mmol) in three portions in 4 h. After stirring for an additional 4 h, the reaction was concentrated to ˜350 ml and extracted with EtOAc (4×80 mL). The combined organic layers were washed with brine (100 mL), dried (MgSO₄), filtered, concentrated and purified by SiO₂ column chromatography (1:1 hexanes/EtOAc) to give the title compound (22.54 g, 86% yield). ESI MS m/z 268.2 ([M+Na]⁺).

### Example 207. Synthesis of (S)-1-tert-butyl 2-methyl 4-oxopyrrolidine-1,2-dicarboxylate

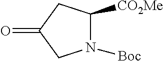

The title compound prepared through Dess-Martin oxidation was described in: Franco Manfre et al. J. Org. Chem. 1992, 57, 2060-2065. Alternatively Swern oxidation procedure is as following: To a solution of (COCl)₂ (13.0 ml, 74.38 mmol) in CH₂Cl₂ (350 ml) cooled to −78° C. was added dry DMSO (26.0 mL). The solution was stirred at −78° C. for 15 min and then (2S,4R)-1-tert-butyl 2-methyl 4-hydroxypyrrolidine-1,2-dicarboxyl ate (8.0 g, 32.63 mmol) in CH₂Cl₂ (100 ml) was added. After stirring at −78° C. for 2 h, triethylamine (50 ml, 180.3 mmol) was added dropwise, and the reaction solution was warmed to room temperature. The mixture was diluted with aq. NaH₂PO₄ solution (1.0 M, 400 ml) and phases separated. The aqueous layer was extracted with CH₂Cl₂ (2×60 ml). The organic layers were combined, dried over MgSO₄, filtered, concentrated and purified by SiO₂ column chromatography (7:3 hexanes/EtOAc) to give the title compound (6.73 g, 85% yield). ESI MS m/z 266.2 ([M+Na]⁺).

### Example 208. Synthesis of (S)-1-tert-butyl 2-methyl 4-methylenepyrrolidine-1,2-dicarboxylate

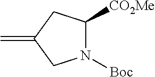

To a suspension of methyltriphenylphosphonium bromide (19.62 g, 55.11 mmol) in THF (150 mL) at 0° C. was added potassium-t-butoxide (6.20 g, 55.30 mmol) in anhydrous THF (80 mL). After stirring at 0° C. for 2 h, the resulting yellow ylide was added to a solution of (S)-1-tert-butyl 2-methyl 4-oxopyrrolidine-1,2-dicarboxylate (6.70 g, 27.55 mmol) in THF (40 mL). After stirring at r.t. for 1 h, the reaction mixture was concentrated, diluted with EtOAc (200 mL), washed with H₂O (150 mL), brine (150 mL), dried over MgSO₄, concentrated and purified on SiO₂ column chromatography (9:1 hexanes/EtOAc) to yield the title compound (5.77 g, 87% yield). EI MS m/z 264 ([M+Na]⁺).

### Example 209. Synthesis of (S)-methyl 4-methylenepyrrolidine-2-carboxylate Hydrochloride

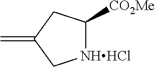

To a solution of (S)-1-tert-butyl 2-methyl 4-methylenepyrrolidine-1,2-dicarboxylate (5.70 g, 23.63 mmol) in EtOAc (40 ml) at 4° C. was added HCl (12 M, 10 ml). The mixture was stirred for 1 h, diluted with toluene (50 ml), concentrated, and crystallized with EtOH/hexane to yield the title compound as HCl salt (3.85 g, 92% yield). EI MS m/z 142.2 ([M+H]⁺).

### Example 210. Synthesis of (S)-tert-butyl 2-(hydroxymethyl)-4-methylenepyrrolidine-1-carboxylate

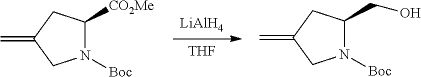 [SMILES: C=C1C[C@@H](C(=O)OC)N(C(=O)OC(C)(C)C)C1 ; "LiAlH4" ; C=C1C[C@@H](CO)N(C(=O)OC(C)(C)C)C1 ; "THF"]

To a solution of (S)-1-tert-butyl 2-methyl 4-methylenepyrrolidine-1,2-dicarboxylate. (5.20 g, 21.56 mmol) in anhydrous THF (100 mL) at 0° C. was added LiAlH₄ (15 ml, 2M in THF). After stirring at 0° C. for 4 h, the reaction was quenched by addition of methanol (5 ml) and water (20 ml). The reaction mixture was neutralized with 1 M HCl to pH 7, diluted with EtOAc (80 ml), filtered through Celite, separated and the aqueous layer was extracted with EtOAc. The organic layers were combined, dried over Na₂SO₄, concentrated and purified on SiO₂ column chromatography (1:5 EtOAc/DCM) to yield the title compound (3.77 g, 82% yield). EI MS m/z 236.40 ([M+Na]⁺).

### Example 211. Synthesis of (S)-(4-methylenepyrrolidin-2-yl)methanol, Hydrochloride Salt

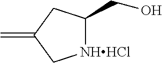

To a solution of (S)-tert-butyl 2-(hydroxymethyl)-4-methylenepyrrolidine-1-carboxylate (3.70 g, 17.36 mmol) in EtOAc (30 ml) at 4° C. was added HCl (12 M, 10 ml). The mixture was stirred for 1 h, diluted with toluene (50 ml), concentrated, and crystallized with EtOH/hexane to yield the title compound as HCl salt (2.43 g, 94% yield). EI MS m/z 115.1 ([M+H]⁺).

### Example 212. Synthesis of 4-(benzyloxy)-3-methoxybenzoic Acid

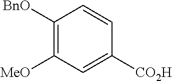 [SMILES: COc1cc(C(=O)O)ccc1OCc1ccccc1]

To a mixture of 4-hydroxy-3-methoxybenzoic acid (50.0 g, 297.5 mmol) in ethanol (350 ml) and aq. NaOH solution (2.0 M, 350 ml) was added BnBr (140.0 g, 823.5 mmol). The mixture was stirred at 65° C. for 8 h, concentrated, co-evaporated with water (2×400 ml) and concentrated to ˜400 ml, acidified to pH 3.0 with 6 N HCl. The solid was collected by filtration, crystallized with EtOH, dried at 45° C. under vacuum to afford the title compound (63.6 g, 83% yield). ESI MS m/z 281.2 ([M+Na]⁺).

### Example 213. Synthesis of 4-(benzyloxy)-5-methoxy-2-nitrobenzoic Acid

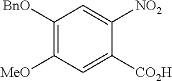 [SMILES: COc1cc(C(=O)O)c([N+](=O)[O-])cc1OCc1ccccc1]

To a solution of 4-(benzyloxy)-3-methoxybenzoic acid (63.5 g, 246.0 mmol) in CH₂Cl₂ (400 ml) and HOAc (100 ml) was added HNO₃ (fuming, 25.0 ml, 528.5 mmol). The mixture was stirred for 6 h, concentrated, crystallized with EtOH, dried at 40° C. under vacuum to afford the title compound (63.3 g, 85% yield). ESI MS m/z 326.1 ([M+Na]⁺).

### Example 214. Synthesis of (S)-(4-(benzyloxy)-5-methoxy-2-nitrophenyl)(2-(hydroxymethyl)-4-methylenepyrrolidin-1-yl)methanone

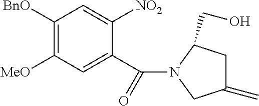 [SMILES: C=C1C[C@@H](CO)N(C(=O)c2cc(OC)c(OCc3ccccc3)cc2[N+](=O)[O-])C1]

A catalytic amount of DMF (30 μl) was added to a solution of 4-(benzyloxy)-5-methoxy-2-nitrobenzoic acid (2.70 g, 8.91 mmol) and oxalyl chloride (2.0 mL, 22.50 mmol) in anhydrous CH₂Cl₂ (70 mL) and the resulting mixture was stirred at room temperature for 2 h. Excess CH₂Cl₂ and oxalyl chloride was removed with rotavap. The acetyl chloride was re-suspended in fresh CH₂Cl₂ (70 mL) and was added slowly to a pre-mixed solution of (S)-(4-methylenepyrrolidin-2-yl)methanol, hydrochloride salt (1.32 g, 8.91 mmol) and Et₃N (6 mL) in CH₂Cl₂ at 0° C. under N₂ atmosphere. The reaction mixture was allowed to warm to r.t. and stirring was continued for 8 h. After removal of CH₂Cl₂ and Et₃N, the residue was partitioned between H₂O and EtOAc (70/70 mL). The aqueous layer was further extracted with EtOAc (2×60 mL). The combined organic layers were washed with brine (40 mL), dried (MgSO₄) and concentrated. Purification of the residue with flash chromatography (silica gel, 2:8 hexanes/EtOAc) yielded the title compound (2.80 g, 79% yield). EI MS m/z 421.2 ([M+Na]⁺).

### Example 215. Synthesis of (S)-(4-(benzyloxy)-5-methoxy-2-nitrophenyl)(2-(((tert-butyldimethylsilyl)oxy)methyl)-4-methylenepyrrolidin-1-yl)methanone

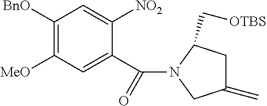 [SMILES: C=C1C[C@@H](CO[Si](C)(C)C(C)(C)C)N(C(=O)c2cc(OC)c(OCc3ccccc3)cc2[N+](=O)[O-])C1]

(S)-(4-(Benzyloxy)-5-methoxy-2-nitrophenyl)(2-(hydroxymethyl)-4-methylenepyrrolidin-1-yl)methanone (2.78 g, 8.52 mmol) in the mixture of DCM (10 ml) and pyridine (10 ml) was added tert-butylchlorodimethylsilane (2.50 g, 16.66 mmol). The mixture was stirred for overnight, concentrated and purified on SiO₂ column eluted with EtOAc/CH₂Cl₂ (1:6) to afford the title compound (3.62 g, 83% yield, ˜95% pure). MS ESI m/z calcd for C₂₇H₃₇N₂O₆Si [M+H]⁺ 513.23. found 513.65.

### Example 216. Synthesis of (S)-(4-hydroxy-5-methoxy-2-nitrophenyl)(2-(hydroxymethyl)-4-methylenepyrrolidin-1-yl)methanone

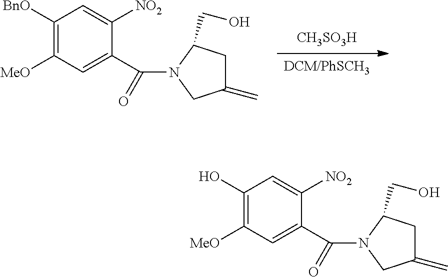 [SMILES: C=C1C[C@@H](CO)N(C(=O)c2cc(OC)c(OCc3ccccc3)cc2[N+](=O)[O-])C1 ; "CH3SO3H" ; "DCM/PhSCH3" ; C=C1C[C@@H](CO)N(C(=O)c2cc(OC)c(O)cc2[N+](=O)[O-])C1]

(S)-(4-(Benzyloxy)-5-methoxy-2-nitrophenyl)(2-(hydroxymethyl)-4-methylenepyrrolidin-1-yl)methanone (2.80 g, 7.03 mmol) in the mixture of DCM (30 ml) and CH₃SO₃H (8 ml) was added PhSCH₃ (2.00 g, 14.06 mmol). The mixture was stirred for 0.5 h, diluted with DCM (40 ml), neutralized with carefully addition of 0.1 M Na₂CO₃ solution. The mixture was separated and the aqueous solution was extracted with DCM (2×10 ml). The organic layers were combined, dried over Na₂SO₄, concentrated and purified on SiO₂ column eluted with MeOH/CH₂Cl₂ (1:15 to 1:6) to afford the title compound (1.84 g, 85% yield, ˜95% pure). MS ESI m/z calcd for C₁₄H₁₇N₂O₆ [M+H]⁺ 309.10. found 309.30.

### Example 217. Synthesis of (S)-((pentane-1,5-diylbis(oxy))bis(5-methoxy-2-nitro-4,1-phenylene))bis(((S)-2-(hydroxymethyl)-4-methylenepyrrolidin-1-yl)methanone)

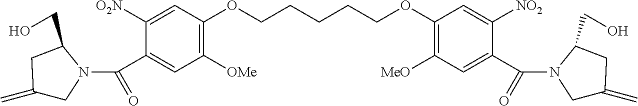

(S)-(4-hydroxy-5-methoxy-2-nitrophenyl)(2-(hydroxymethyl)-4-methylenepyrrolidin-1-yl)methanone (0.801 g, 2.60 mmol) in butanone (10 ml) was added Cs₂CO₃, (2.50 g, 7.67 mmol), followed by addition of 1,5-diiodopentane (415 mmol, 1.28 mmol). The mixture was stirred for 26 h, concentrated and purified on SiO₂ column eluted with MeOH/CH₂Cl₂ (1:15 to 1:5) to afford the title compound (0.675 g, 77% yield, ˜95% pure). MS ESI m/z calcd for C₃₃H₄₁N₄O₁₂ [M+H]⁺ 685.26. found 685.60.

### Example 218. Synthesis of (S)-((pentane-1,5-diylbis(oxy))bis(2-amino-5-methoxy-4,1-phenylene))bis(((S)-2-(hydroxymethyl)-4-methylenepyrrolidin-1-yl)methanone)

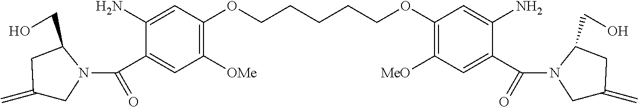

(S)-((pentane-1,5-diylbis(oxy))bis(5-methoxy-2-nitro-4,1-phenylene))bis(((S)-2-(hydroxymethyl)-4-methylenepyrrolidin-1-yl)methanone) (0.670 g, 0.98 mmol) in CH₃OH (10 ml) was added Na₂S20₄ (1.01 g, 5.80 mmol) in H₂O (8 ml). The mixture was stirred at room temperature for 30 h. The reaction mixture was evaporated and co-evaporated with DMA (2×10 mL) and EtOH (2×10 ml) under high vacuum to dryness to afford the title compound (total weight 1.63 g) containing inorganic salts which was used directly for the next step reaction (without further separation). EI MS m/z 647.32 ([M+Na]⁺).

### Example 219. Synthesis of C-1 (a PBD Dimer Analog Having a Bis-Linker)

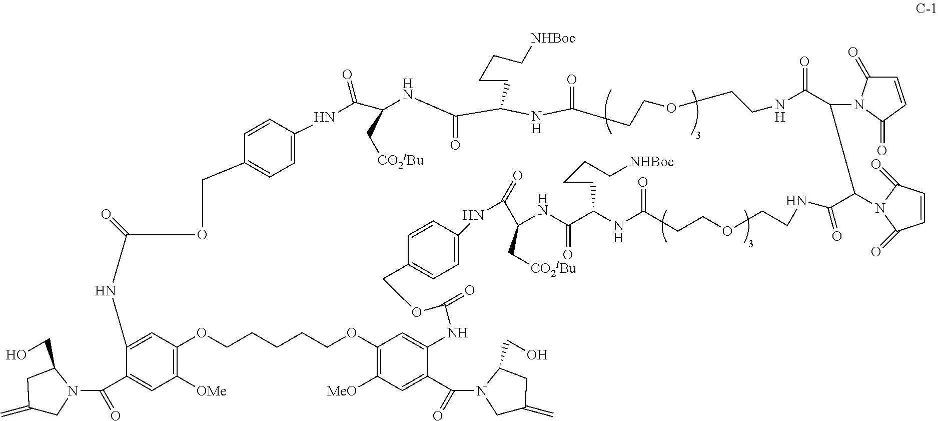

(3S,6S,39S,42S)-di-tert-butyl 6,39-bis(4-((tert-butoxycarbonyl)amino)butyl)-22,23-bis(2,5-dioxo-2,5-dihydro-1H-pyrrol-1-yl)-3,42-bis((4-(hydroxymethyl)phenyl)carbamoyl)-5,8,21,24,37,40-hexaoxo-11,14,17,28,31,34-hexaoxa-4,7,20,25,38,41-hexaazatetratetracontane-1,44-dioate (0.840 g, 0.488 mmol) in THF (8 mL) containing pyridine (0.100 ml, 1.24 mmol) at 0° C. was added dropwise of a solution of triphosgene (0.290 mg, 0.977 mmol) in THF (3.0 mL). The reaction mixture was stirred at 0° C. for 15 min then was used directly in the next step.

(S)-((pentane-1,5-diylbis(oxy))bis(2-amino-5-methoxy-4,1-phenylene))bis(((S)-2-(hydroxymethyl)-4-methylenepyrrolidin-1-yl)methanone) containing inorganic salts (0.842 mg, ˜0.49 mmol) was suspended in EtOH (10 ml) at 0° C. was added the trichloride in THF prepared above. The mixture was stirred at 0° C. for 4 h, then warmed to RT for 1 h, concentrated, and purified by reverse phase HPLC (250 (L) mm×10(d) mm, C₁g column, 10-80% acetonitrile/water in 40 min, v=8 ml/min) to afford the title compound (561.1 mg, 48% yield in three steps). ESI MS m/z: calcd for C₁₁₇H₁₆₃N₁₆O₃₈ [M+H]⁺ 2400.12. found 2400.90.

### Example 220. Synthesis of C-2 (a PBD Dimer Analog Having a Bis-Linker)

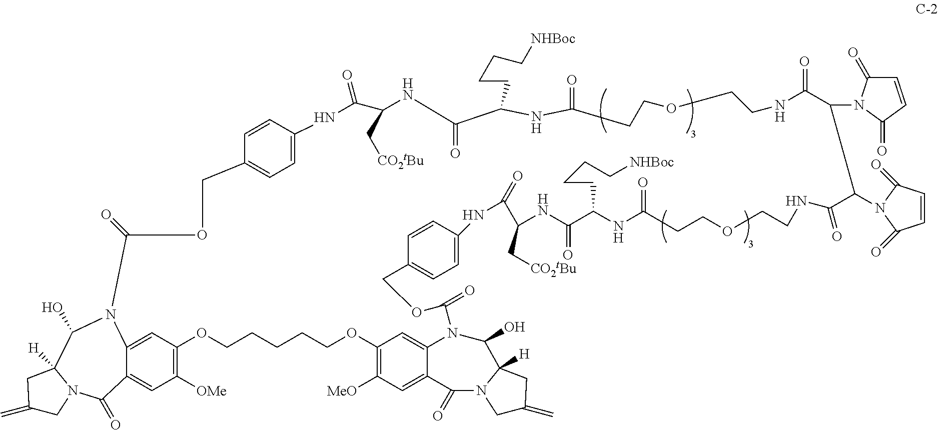

Dess-Martin periodinane (138.0 mg, 0.329 mmol) was added to a solution of compound C-1 (132.0 mg, 0.055 mmol) in DCM (5.0 mL) at 0° C. The reaction mixture was warmed to RT and was stirred for 2 h. A saturated solution of NaHCO₃/Na₂SO₃ (5.0 mL/5.0 mL) was then added and the mixture was extracted with DCM (3×25 mL). The combined organic layers were washed with NaHCO₃/Na₂SO₃ (5.0 mL/5.0 mL), brine (10 mL), dried over Na₂SO₄, filtered, concentrated and purified by reverse phase HPLC (250 (L) mm×10(d) mm, C₁₈ column, 10-80% acetonitrile/water in 40 min, v=8 ml/min) to afford the title compound (103.1 mg, 78% yield) as a foam. ESI MS m/z: calcd for C₁₁₇H₁₅₈N₁₆O₃₈ [M+H]⁺ 2396.09. found 2396.65.

### Example 221. Synthesis of C-3 (a PBD Dimer Analog Having a Bis-Linker)

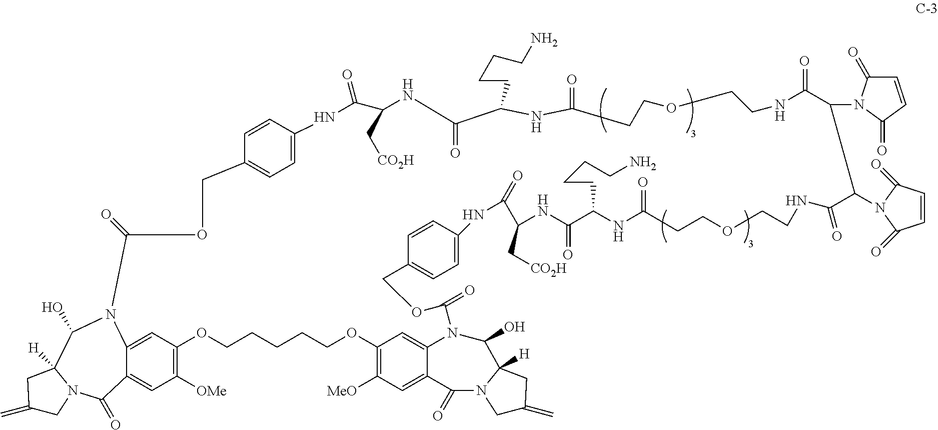

C-2 compound (55.0 mg, 0.023 mmol) was dissolved in DCM (3 ml), followed by addition of TFA (3 ml). The reaction mixture was stirred at RT for 2 h, then concentrated, and co-evaporated with DCM/toluene to dryness to afford the crude product C-3 (48.0 mg, 100% yield, 92% pure by HPLC) which was further purified by reverse phase HPLC (250 (L) mm×10(d) mm, C₁₈ column, 5-60% acetonitrile/water in 40 min, v=8 ml/min) to afford the pure product C-3 (42.1 mg, 88% yield, 96% pure) as a foam. ESI MS m/z: calcd for C₉₉H₁₂₆N₁₆O₃₄ [M+H]⁺ 2083.86. found 2084.35.

### Example 222. Synthesis of (S)-methyl 1-(4-(benzyloxy)-5-methoxy-2-nitrobenzoyl)-4-methylenepyrrolidine-2-carboxylate

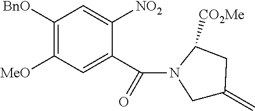 [SMILES: C=C1C[C@@H](C(=O)OC)N(C(=O)c2cc(OC)c(OCc3ccccc3)cc2[N+](=O)[O-])C1]

A catalytic amount of DMF (30 μl) was added to a solution of 4-(benzyloxy)-5-methoxy-2-nitrobenzoic acid (2.70 g, 8.91 mmol) and oxalyl chloride (2.0 mL, 22.50 mmol) in anhydrous CH₂Cl₂ (70 mL) and the resulting mixture was stirred at room temperature for 2 h. Excess CH₂Cl₂ and oxalyl chloride was removed with rotavap. The acetyl chloride was re-suspended in fresh CH₂Cl₂ (70 mL) and was added slowly to a pre-mixed solution of (S)-methyl 4-methylenepyrrolidine-2-carboxylate hydrochloride (1.58 g, 8.91 mmol) and Et₃N (6 mL) in CH₂Cl₂ at 0° C. under N₂ atmosphere. The reaction mixture was allowed to warm to r.t. and stirring was continued for 8 h. After removal of CH₂Cl₂ and Et₃N, the residue was partitioned between H₂O and EtOAc (70/70 mL). The aqueous layer was further extracted with EtOAc (2×60 mL). The combined organic layers were washed with brine (40 mL), dried (MgSO₄) and concentrated. Purification of the residue with flash chromatography (silica gel, 2:8 hexanes/EtOAc) yielded the title compound (2.88 g, 76% yield). EI MS m/z 449.1 ([M+Na]⁺).

### Example 223. Synthesis of (S)-1-(4-(benzyloxy)-5-methoxy-2-nitrobenzoyl)-4-methylenepyrro-lidine-2-carbaldehyde

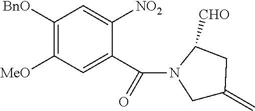 [SMILES: C=C1C[C@@H](C=O)N(C(=O)c2cc(OC)c(OCc3ccccc3)cc2[N+](=O)[O-])C1]

To a vigorously stirred solution of (S)-methyl 1-(4-(benzyloxy)-5-methoxy-2-nitro benzoyl)-4-methylenepyrrolidine-2-carboxylate (2.80 g, 6.57 mmol) in anhydrous CH₂Cl₂ (60 mL) was added DIBAL-H (1N in CH₂Cl₂, 10 mL) dropwise at −78° C. under N₂ atmosphere. After the mixture was stirred for an additional 90 min, excess reagent was decomposed by addition of 2 ml of methanol, followed by 5% HCl (10 mL). The resulting mixture was allowed to warm to 0° C. Layers were separated and the aqueous layer was further extracted with CH₂Cl₂ (3×50 mL). Combined organic layers were washed with brine, dried (MgSO₄) and concentrated. Purification of the residue with flash chromatography (silica gel, 95:5 CHCl₃/MeOH) yielded the title compound (2.19 g, 84% yield). EIMS m/z 419.1 ([M+Na]⁺).

### Example 224. Synthesis of (S)-8-(benzyloxy)-7-methoxy-2-methylene-2,3-dihydro-1H-benzo[e]-pyrrolo[1,2-a]azepin-5(11aH)-one

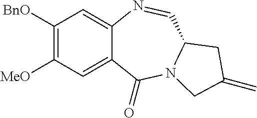 [SMILES: C=C1C[C@H]2C=Nc3cc(OCc4ccccc4)c(OC)cc3C(=O)N2C1]

A mixture of (S)-1-(4-(benzyloxy)-5-methoxy-2-nitrobenzoyl)-4-methylenepyrro-lidine-2-carbaldehyde (2.18 g, 5.50 mmol) and Na₂S20₄ (8.0 g, 45.97 mmol) in THF (60 ml) and H₂O (40 ml) was stirred at room temperature for 20 h. Solvents were removed under high vacuum. The residue was re-suspended in MeOH (60 mL), and HCl (6M) was added dropwise until pH ˜2 was reached. The resulting mixture was stirred at r.t. for 1 h. The reaction was worked-up by removing most of MeOH, then diluted with EtOAc (100 mL). The EtOAc solution was washed with sat. NaHCO₃, brine, dried (MgSO₄), and concentrated. Purification of the residue with flash chromatography (silica gel, 97:3 CHCl₃/MeOH) yielded the title compound (1.52 g, 80%). EIMS m/z 372.1 ([M+Na]⁺).

### Example 225. Synthesis of (S)-8-hydroxy-7-methoxy-2-methylene-2,3-dihydro-1H-benzo[e]-pyrrolo[1,2-a]azepin-5(11aH)-one

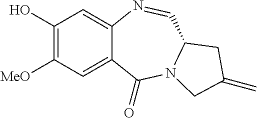 [SMILES: C=C1C[C@H]2C=Nc3cc(O)c(OC)cc3C(=O)N2C1]

To a solution of (S)-8-(benzyloxy)-7-methoxy-2-methylene-2,3-dihydro-1H-benzo[e]-pyrrolo[1,2-a]azepin-5(11aH)-one (1.50 g, 4.32 mmol) in 70 ml of CH₂C1₂ was added 25 ml of CH₃SO₃H at 0° C. The mixture was stirred at 0° C. for 10 min then r.t. for 2 h, diluted with CH₂Cl₂, pH adjusted with cold 1.0 N NaHCO₃ to 4 and filtered. The aqueous layer was extracted with CH₂Cl₂ (3×60 ml). The organic layers were combined, dried over Na₂SO₄, filtered, evaporated and purified on SiO₂ column chromatography (CH₃OH/CH₂Cl₂ 1:15) to afford 811 mg (73% yield) of the title product. EIMS m/z 281.1 ([M+Na]⁺).

### Example 226. Synthesis of (11aS,11a′S)-8,8′-(pentane-1,5-diylbis(oxy))bis(7-methoxy-2-methylene-2,3-dihydro-1H-benzo[e]pyrrolo[1,2-a][1,4]diazepin-5(11aH)-one)

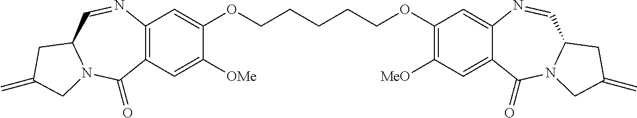

To a stirred suspended solution of Cs₂CO₃ (0.761 g, 2.33 mmol) in butanone (8 ml) were added (S)-8-hydroxy-7-methoxy-2-methylene-2,3-dihydro-1H-benzo[e]-pyrrolo[1,2-a]azepin-5(11aH)-one (401 mg, 1.55 mmol) and 1,5-diiodopentane (240 mg, 0.740 mmol). The mixture was stirred at r.t. overnight, concentrated, and purified on SiO₂ chromatography (EtOAc/CH₂Cl₂ 1:10) to afford 337 mg (78% yield) of the title product. EIMS m/z 607.2 ([M+Na]⁺).

### Example 227. Synthesis of (S)-7-methoxy-8-((5-(((S)-7-methoxy-2-methylene-5-oxo-2,3,5,10,11,11a-hexahydro-1H-benzo[e]pyrrolo[1,2-a][1,4]diazepin-8-yl)oxy)pentyl)oxy)-2-methylene-2,3-dihydro-1H-benzo[e]pyrrolo[1,2-a][1,4]diazepin-5(11aH)-one

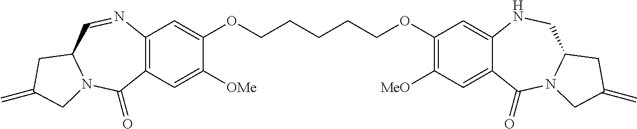

To a solution of (11aS,11a′S)-8,8′-(pentane-1,5-diylbis(oxy))bis(7-methoxy-2-methylene-2,3-dihydro-1H-benzo[e]pyrrolo[1,2-a][1,4]diazepin-5(11aH)-one) (150 mg, 0.256 mmol) in anhydrous dichloromethane (1 mL) and absolute ethanol (1.5 mL) was added sodium borohydride in methoxyethyl ether (85 μl, 0.5 M, 0.042 mmol) at 0° C. The ice bath was removed after 5 minutes and the mixture was stirred at room temperature for 3 hours, then cooled to 0° C., quenched with saturated ammonium chloride, diluted with dichloromethane, and phases separated. The organic layer was washed with brine, dried over anhydrous Na₂SO₄, filtered through Celite and concentrated. The residue was purified by reverse phase HPLC (C₁₈ column, acetonitrile/water). The corresponding fractions were extracted with dichloromethane and concentrated to afford the title compound (64.7 mg, 43%), MS m/z 609.2 ([M+Na]⁺), 625.3 ([M+K]⁺) and 627.2 ([M+Na+H₂O]⁺); the fully reduced compound was obtained (16.5 mg, 11%), MS m/z 611.2 ([M+Na]⁺), 627.2 ([M+K]⁺), 629.2 ([M+Na+H₂O]⁺); and the unreacted starting material was also recovered (10.2 mg, 7%), MS m/z 607.2 ([M+Na]⁺), 625.2 ([M+Na+H₂O]⁺).

### Example 228. Synthesis of (S)-8-((5-(((S)-10-(3-(2-(2-azidoethoxy)ethoxy) propanoyl)-7-methoxy-2-methylene-5-oxo-2,3,5,10,11,11a-hexahydro-1H-benzo[e]pyrrolo[1,2-a][1,4]diazepin-8-yl)oxy)pentyl)oxy)-7-methoxy-2-methylene-2,3-dihydro-1H-benzo[e]pyrrolo[1,2-a][1,4]diazepin-5(11aH)-one

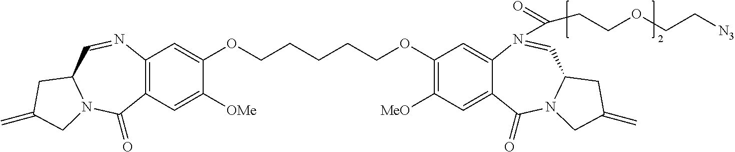

To the mixture of (S)-7-methoxy-8-((5-(((S)-7-methoxy-2-methylene-5-oxo-2,3,5,10,11,11a-hexahydro-1H-benzo[e]pyrrolo[1,2-a][1,4]diazepin-8-yl)oxy)pentyl)oxy)-2-methylene-2,3-dihydro-1H-benzo[e]pyrrolo[1,2-a][1,4]diazepin-5(11aH)-one (60.0 mg, 0.102 mmol) and 2,5-dioxopyrrolidin-1-yl 3-(2-(2-azidoethoxy)ethoxy)propanoate (40.5 mg, 0.134 mmol) in dichloromethane (5 ml) was added EDC (100.5 mg, 0.520 mmol). The mixture was stirred at r.t. overnight, concentrated and purified on SiO₂ column chromatography (EtOAc/CH₂Cl₂, 1:6) to afford 63.1 mg (81% yield) of the title product. ESI MS m/z C₄₀H₅₀N₇O₉[M+H]⁺, cacld. 772.36. found 772.30.

### Example 229. Synthesis of (S)-8-((5-(((S)-10-(3-(2-(2-aminoethoxy)ethoxy) propanoyl)-7-methoxy-2-methylene-5-oxo-2,3,5,10,11,11a-hexahydro-1H-benzo[e]pyrrolo[1,2-a][1,4]diazepin-8-yl)oxy)pentyl)oxy)-7-methoxy-2-methylene-2,3-dihydro-1H-benzo[e]pyrrolo[1,2-a][1,4]diazepin-5(11aH)-one

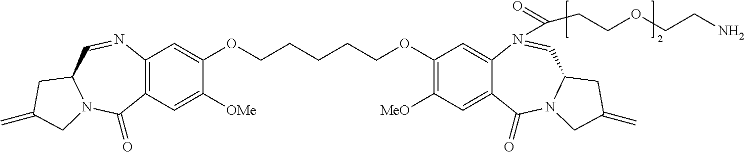

To a solution of (S)-8-((5-(((S)-10-(3-(2-(2-azidoethoxy)ethoxy) propanoyl)-7-methoxy-2-methylene-5-oxo-2,3,5,10,11,11a-hexahydro-1H-benzo[e]pyrrolo[1,2-a][1,4]diazepin-8-yl)oxy)pentyl)oxy)-7-methoxy-2-methylene-2,3-dihydro-1H-benzo[e]pyrrolo[1,2-a][1,4]diazepin-5(11aH)-one (60 mg, 0.078 mmol) in the mixture of THF (5 ml) and NaH₂PO₄ buffer solution (pH 7.5, 1.0 M, 0.7 ml) was added PPh₃ (70 mg, 0.267 mmol). The mixture was stirred at r.t. overnight, concentrated and purified on C₁₈ preparative HPLC, eluted with water/CH₃CN (from 90% water to 35% water in 35 min) to afford 45.1 mg (79% yield) of the title product after drying under high vacuum. ESI MS m/z C₄₀H₅₂N₅O₉ [M+H]⁺, cacld. 746.37, found 746.50.

### Example 230. Synthesis of (S)—N-(2-((S)-8-((5-(((11S,11aS)-10-((S)-15-azido-5-isopropyl-4,7-dioxo-10,13-dioxa-3,6-diazapentadecan-1-oyl)-11-hydroxy-7-methoxy-2-methylene-5-oxo-2,3,5,10,11,11a-hexahydro-1H-benzo[e]pyrrolo[1,2-a][1,4]diazepin-8-yl)oxy)pentyl)-oxy)-7-methoxy-2-methylene-5-oxo-2,3,11,11a-tetrahydro-1H-benzo[e]pyrrolo[1,2-a][1,4]diazepin-10(5H)-yl)-2-oxoethyl)-2-(3-(2-(2-azidoethoxy)ethoxy)propanamido)-3-methylbutanamide

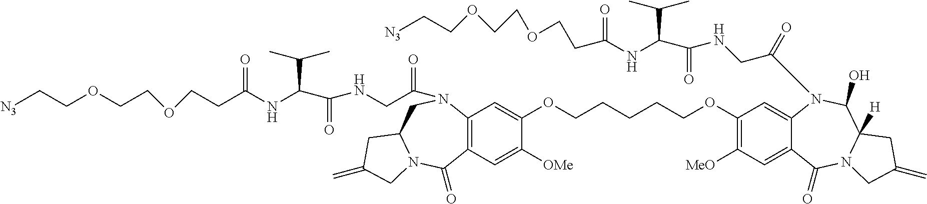 [SMILES: C=C1C[C@H]2CN(C(=O)CNC(=O)[C@@H](NC(=O)CCOCCOCCN=[N+]=[N-])C(C)C)c3cc(OCCCCCOc4cc5c(cc4OC)C(=O)N4CC(=C)C[C@H]4[C@H](O)N5C(=O)CNC(=O)[C@@H](NC(=O)CCOCCOCCN=[N+]=[N-])C(C)C)c(OC)cc3C(=O)N2C1]

To the mixture of (S)-7-methoxy-8-((5-(((S)-7-methoxy-2-methylene-5-oxo-2,3,5,10,11,11a-hexahydro-1H-benzo[e]pyrrolo[1,2-a][1,4]diazepin-8-yl)oxy)pentyl)oxy)-2-methylene-2,3-dihydro-1H-benzo[e]pyrrolo[1,2-a][1,4]diazepin-5(11aH)-one (60.0 mg, 0.102 mmol) and (S)-15-azido-5-isopropyl-4,7-dioxo-10,13-dioxa-3,6-diazapentadecan-1-oic acid (90.2 mg, 0.25 mmol) in DMA (8 ml) was added BrOP (240.2 mg, 0.618 mmol). The mixture was stirred at r.t. overnight, concentrated and purified on SiO₂ column chromatography (CH₃OH/CH₂Cl₂, 1:10 to 1:5) to afford 97.1 mg (74% yield) of the title product. ESI MS m/z C₆₁H₈₇N₁₄O₁₇ [M+H]⁺, cacld. 1287.63. found 1287.95.

### Example 231. Synthesis of (S)—N-(2-((S)-8-((5-(((11S,11 aS)-10-((S)-15-amino-5-isopropyl-4,7-dioxo-10,13-dioxa-3,6-diazapentadecan-1-oyl)-11-hydroxy-7-methoxy-2-methylene-5-oxo-2,3,5,10,11,11a-hexahydro-1H-benzo[e]pyrrolo[1,2-a][1,4]diazepin-8-yl)oxy)pentyl)oxy)-7-methoxy-2-methylene-5-oxo-2,3,11,11a-tetrahydro-1H-benzo[e]-pyrrolo[1,2-a][1,4]diazepin-10(5H)-yl)-2-oxoethyl)-2-(3-(2-(2-aminoethoxy)ethoxy)-propanamido)-3-methylbutanamide (C-4)

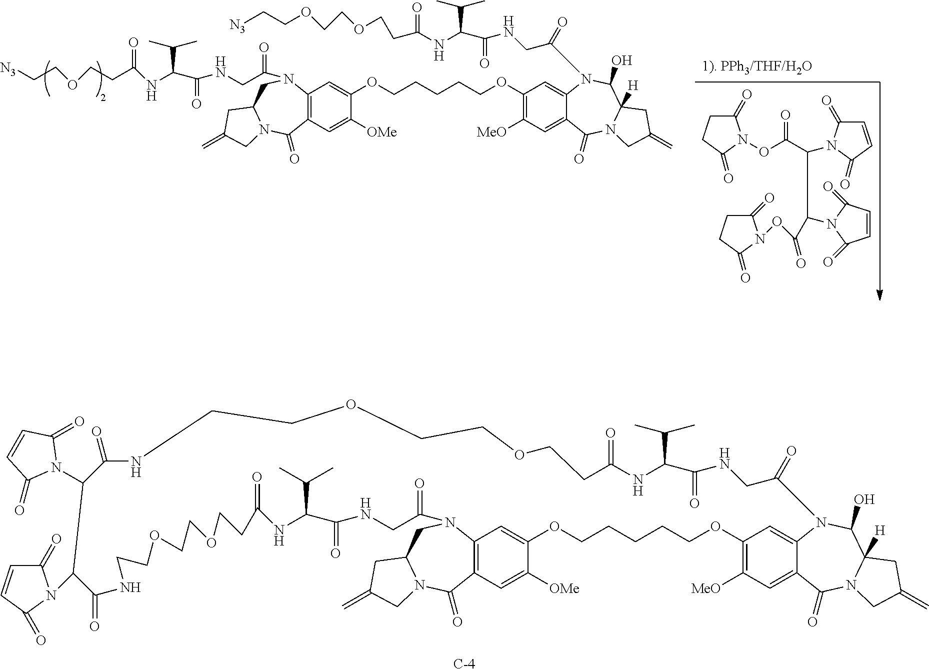

To a solution of (S)—N-(2-((S)-8-((5-(((11S,11aS)-10-((S)-15-azido-5-isopropyl-4,7-dioxo-10,13-dioxa-3,6-diazapentadecan-1-oyl)-11-hydroxy-7-methoxy-2-methylene-5-oxo-2,3,5,10,11,11a-hexahydro-1H-benzo[e]pyrrolo[1,2-a][1,4]diazepin-8-yl)oxy)pentyl)-oxy)-7-methoxy-2-methylene-5-oxo-2,3,11,11a-tetrahydro-1H-benzo[e]pyrrolo[1,2-a][1,4]diazepin-10(5H)-yl)-2-oxoethyl)-2-(3-(2-(2-azidoethoxy)ethoxy)propanamido)-3-methylbutanamide (85 mg, 0.066 mmol) in the mixture of THF (5 ml) and NaH₂PO₄ buffer solution (pH 7.5, 1.0 M, 0.7 ml) was added PPh₃ (100 mg, 0.381 mmol). The mixture was stirred at r.t. overnight. After confirmed by LC-MS to form (S)—N-(2-((S)-8-((5-(((11S,11 aS)-10-((S)-15-amino-5-isopropyl-4,7-dioxo-10,13-dioxa-3,6-diazapentadecan-1-oyl)-11-hydroxy-7-methoxy-2-methylene-5-oxo-2,3,5,10,11,11a-hexahydro-1H-benzo[e]pyrrolo[1,2-a][1,4]diazepin-8-yl)oxy)pentyl)oxy)-7-methoxy-2-methylene-5-oxo-2,3,11,11a-tetrahydro-1H-benzo[e]pyrrolo[1,2-a][1,4]diazepin-10(5H)-yl)-2-oxoethyl)-2-(3-(2-(2-aminoethoxy)ethoxy)propanamido)-3-methylbutanamide (ESI MS m/z C₆₁H₉₀N₁₀O₁₇ [M+Na]⁺, cacld. 1257.66. found 1257.90), bis(2,5-dioxopyrrolidin-1-yl) 2,3-bis(2,5-dioxo-2,5-dihydro-1H-pyrrol-1-yl)succinate (33 mg, 0.066 mmol) was added. The mixture was continued to stir for 4 h, concentrated and purified on C₁₈ preparative HPLC, eluted with water/CH₃CN (from 90% water to 30% water in 35 min) to afford 40.1 mg (40% yield) of the title product C-4 after drying under high vacuum. ESI MS m/z C₇₃H₉₅N₁₂O₂₃ [M+H]⁺, cacld. 1507.66. found 1507.90.

### Example 232. Synthesis of nitro-α-amanitin

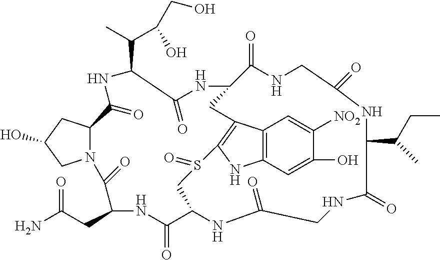 [SMILES: CC[C@H](C)[C@H]1NC(=O)CNC(=O)[C@@H]2Cc3c([nH]c4cc(O)c([N+](=O)[O-])cc34)S(=O)C[C@H](NC(=O)CNC1=O)C(=O)N[C@@H](CC(N)=O)C(=O)N1C[C@H](O)C[C@H]1C(=O)N[C@@H](C(C)[C@@H](O)CO)C(=O)N2]

To a solution of α-amanitin (15.0 mg, 0.0163 mmol) in acetic acid (0.5 mL) and CH₂Cl₂ (1 mL) was added 70% HNO₃ (0.3 mL) at 0° C. The reaction was stirred at 0° C. for 1 h then room temperature 2 h. After water (5 mL) and DMA (4 ml) were, the reaction mixture was concentrated and purified by prep-HPLC (H₂O/MeCN) to give a light yellow solid (9.8 mg, 62% yield). ESI MS m/z: calcd for C₃₉H₅₄N₁₁O₁₆S [M+H]⁺ 963.34. found 964.95.

### Example 233. Synthesis of nitro-β-amanitin

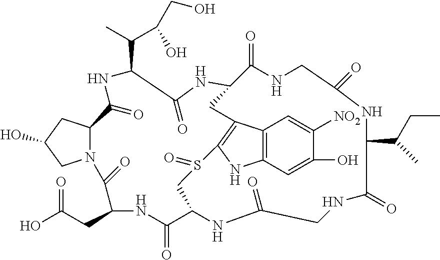 [SMILES: CC[C@H](C)[C@H]1NC(=O)CNC(=O)[C@@H]2Cc3c([nH]c4cc(O)c([N+](=O)[O-])cc34)S(=O)C[C@H](NC(=O)CNC1=O)C(=O)N[C@@H](CC(=O)O)C(=O)N1C[C@H](O)C[C@H]1C(=O)N[C@@H](C(C)[C@@H](O)CO)C(=O)N2]

To a solution of β-amanitin (15.0 mg, 0.0163 mmol) in acetic acid (0.5 mL) and CH₂Cl₂ (1 mL) was added 70% HNO₃ (0.3 mL) at 0° C. The reaction was stirred at 0° C. for 1 h then room temperature 2 h. After water (5 mL) and DMA (4 ml) were added, the reaction mixture was concentrated and purified by prep-HPLC (H₂O/MeCN) to give a light yellow solid (9.8 mg, 62% yield). ESI MS m/z: calcd for C₃₉H₅₃N₁₀O₁₇S [M+H]⁺ 965.32. found 965.86.

### Example 234. Synthesis of a Conjugatable α-Amanitin Analog (D-1) Having a Bis-Linker

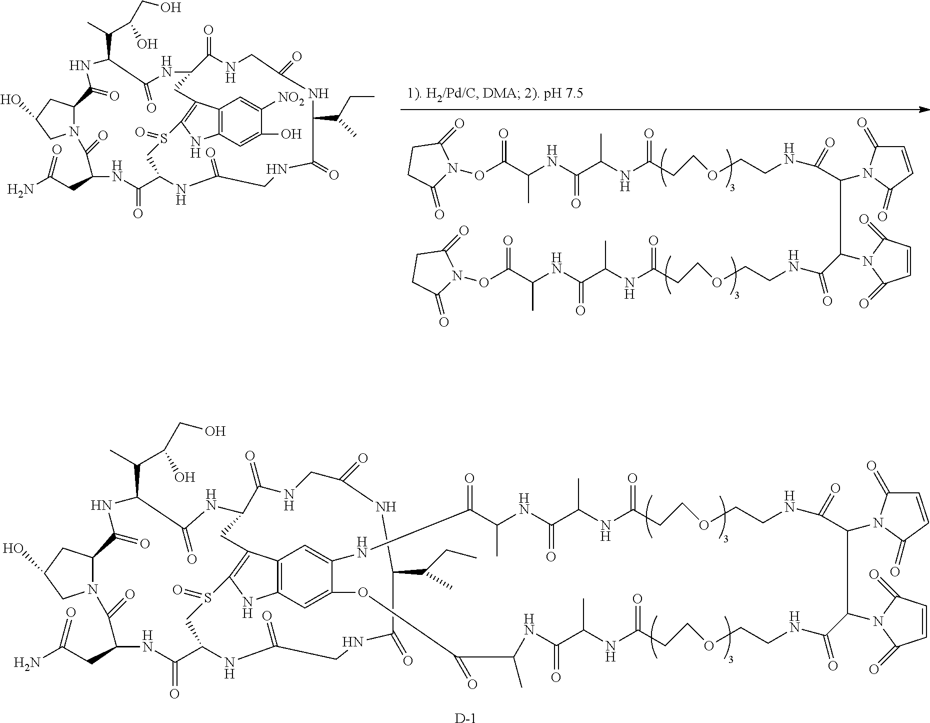

To a solution of nitro-α-amanitin (9.0 mg, 0.0093 mmol) in DMA (1 ml)) was added Pd/C (3 mg, 50% wet), then hydrogenated (1 atm) at room temperature for 6 h. The catalyst 5 was filtered off, followed by addition of 0.5 ml, 0.1 M NaH2PO4, pH 7.5 and bis(2,5-dioxopyrrolidin-1-yl) 21,22-bis(2,5-dioxo-2,5-dihydro-1H-pyrrol-1-yl)-2,5,38,41-tetramethyl-4,7,20,23,36,39-hexaoxo-10,13,16,27,30,33-hexaoxa-3,6,19,24,37,40-hexaazadotetracontane-1,42-dioate (11.0 mg, 0.0092 mmol). The mixture was stirred at r.t. overnight, concentrated and purified on C₁₈ preparative HPLC, eluted with water/CH₃CN (from 90% water to 30% water in 35 min) to afford 6.1 mg (35% yield) of the title product D-1 after drying under high vacuum. ESI MS m/z C₈₁H₁₁₆N₁₉O₃₁S [M+H]⁺, cacld. 1882.77. found 1882.20.

### Example 235. Synthesis of a Conjugatable α-Amanitin Analog (D-1) Having a Bis-Linker

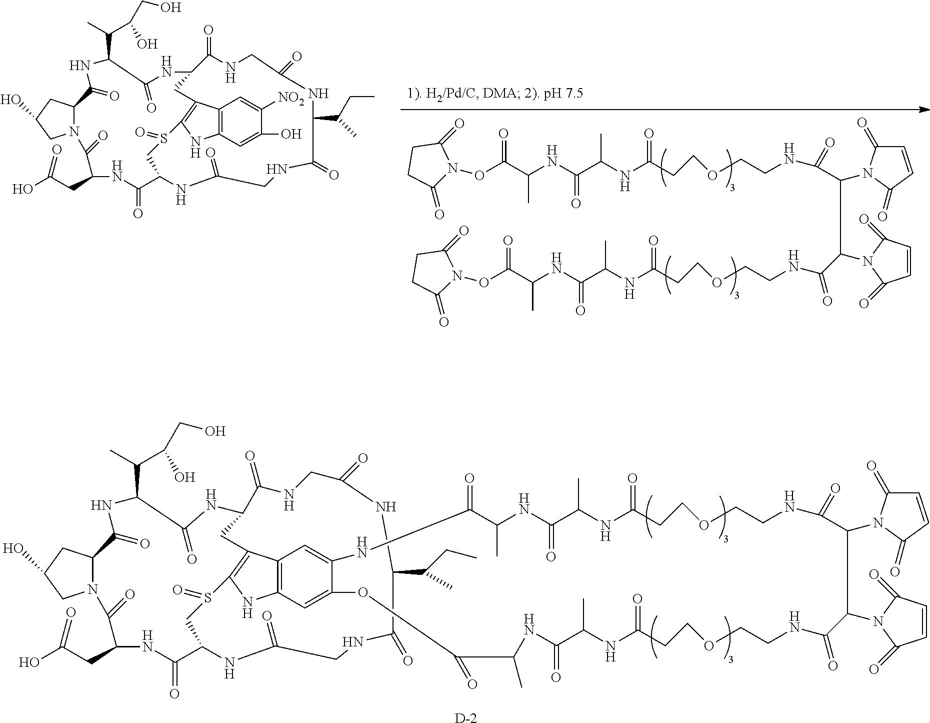

To a solution of nitro-β-amanitin (9.0 mg, 0.0093 mmol) in DMA (1 ml)) was added Pd/C (3 mg, 50% wet), then hydrogenated (1 atm) at room temperature for 6 h. The catalyst was filtered off, followed by addition of 0.5 ml, 0.1 M NaH2PO4, pH 7.5 and bis(2,5-dioxopyrrolidin-1-yl) 21,22-bis(2,5-dioxo-2,5-dihydro-1H-pyrrol-1-yl)-2,5,38,41-tetramethyl-4,7,20,23,36,39-hexaoxo-10,13,16,27,30,33-hexaoxa-3,6,19,24,37,40-hexaazadotetracontane-1,42-dioate (11.0 mg, 0.0092 mmol). The mixture was stirred at r.t. overnight, concentrated and purified on C₁₈ preparative HPLC, eluted with water/CH₃CN (from 90% water to 30% water in 35 min) to afford (7.0 mg 40% yield) of the title product D-2 after drying under high vacuum. ESI MS m/z C₈₁H₁₁₅N₁₈O₃₂S [M+H]⁺, cacld. 1883.76. found 1884.10.

### Example 236. General Method of Preparation of Conjugate

To a mixture of 2.0 mL of 10 mg/ml a her2 antibody in pH 6.0-8.0, were added of 0.70˜2.0 mL PBS buffer of 100 mM NaH₂PO₄, pH 6.5˜8.5 buffers, TCEP (16-20 μL, 20 mM in water) and the compound A-3, A-4, A-5, B-3, B-6, B-9, B-12, B-15, B-18, B-19, B-20, B-21, B-22, B-23, B-24, B-25, B-26, B-28, C-3, C-4, D-1 or D-2 (28-32 μL, 20 mM in DMA,) independently. The mixture was incubated at RT for 4˜18 h, then DHAA (135 μL, 50 mM) was added in. After continuous incubation at RT overnight, the mixture was purified on G-25 column eluted with 100 mM NaH₂PO₄, 50 mM NaCl pH 6.0˜7.5 buffer to afford 12.8-18.1 mg of the conjugate compound A-3a, A-4a, A-5a, B-3a, B-6a, B-9a, B-12a, B-15a, B-18a, B-19a, B-20a, B-21a, B-22a, B-23a, B-24a, B-25a, B-26a, B-28a, C-3a, C-4a, D-1a or D-2a (75%˜90% yield) accordingly in 14.4˜15.5 ml buffer. The drug/antibody ratio (DAR) was 3.1˜4.2 for conjugate which was determined via UPLC-QTOF mass spectrum. It was 94˜99% monomer analyzed by SEC HPLC (Tosoh Bioscience, Tskgel G3000SW, 7.8 mm ID×30 cm, 0.5 ml/min, 100 min) and a single band measured by SDS-PAGE gel. The conjugate structures are displayed below:

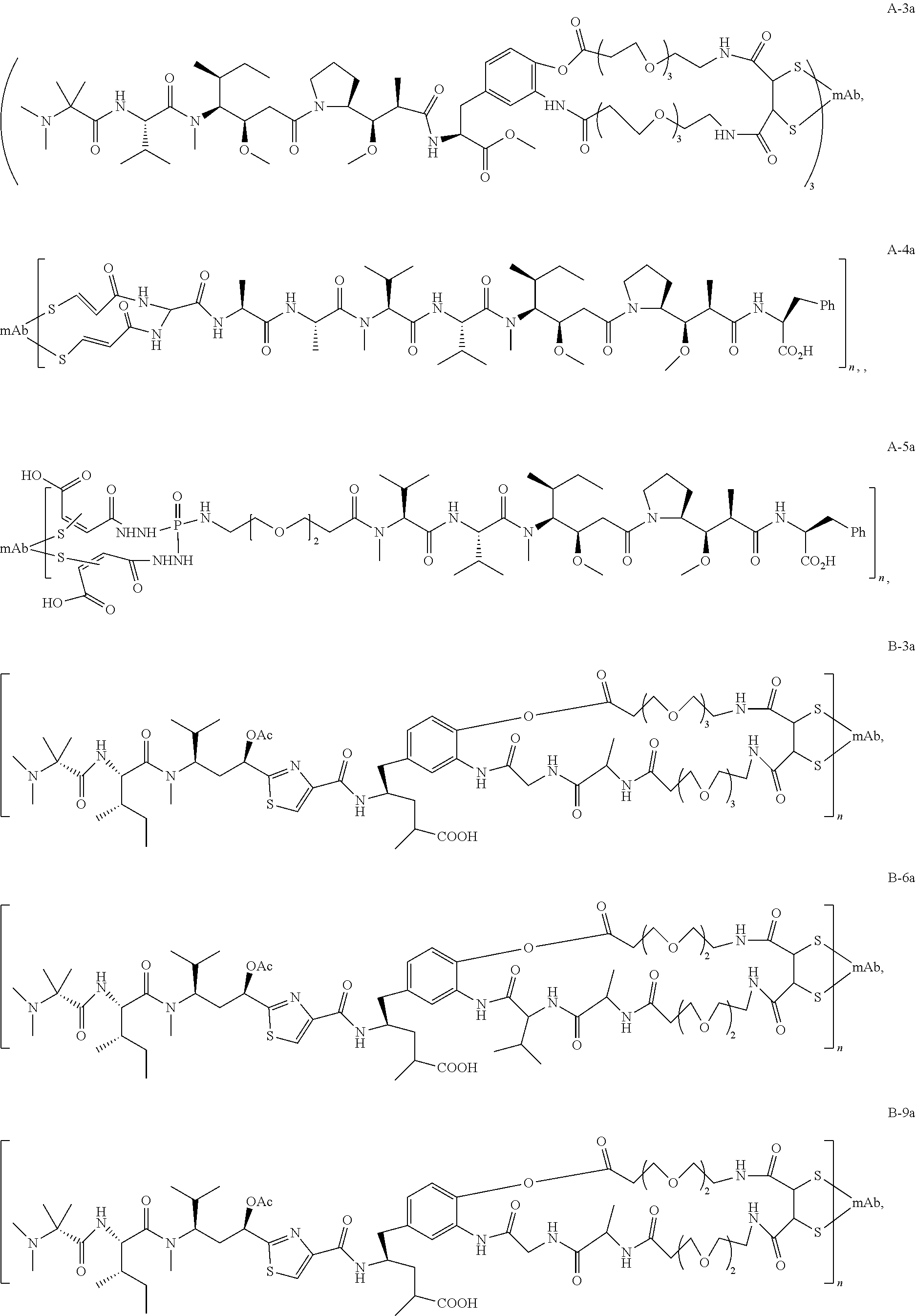

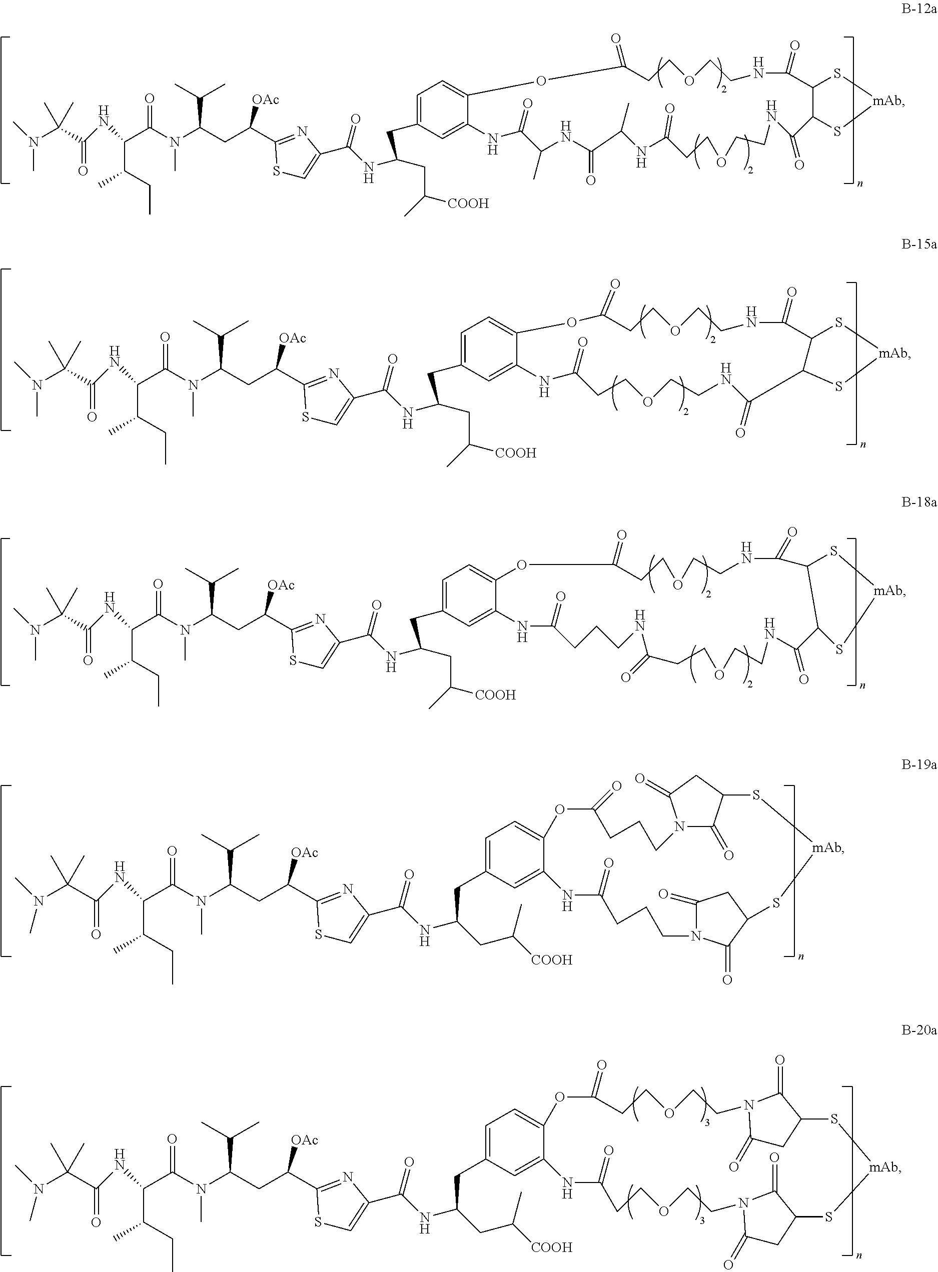

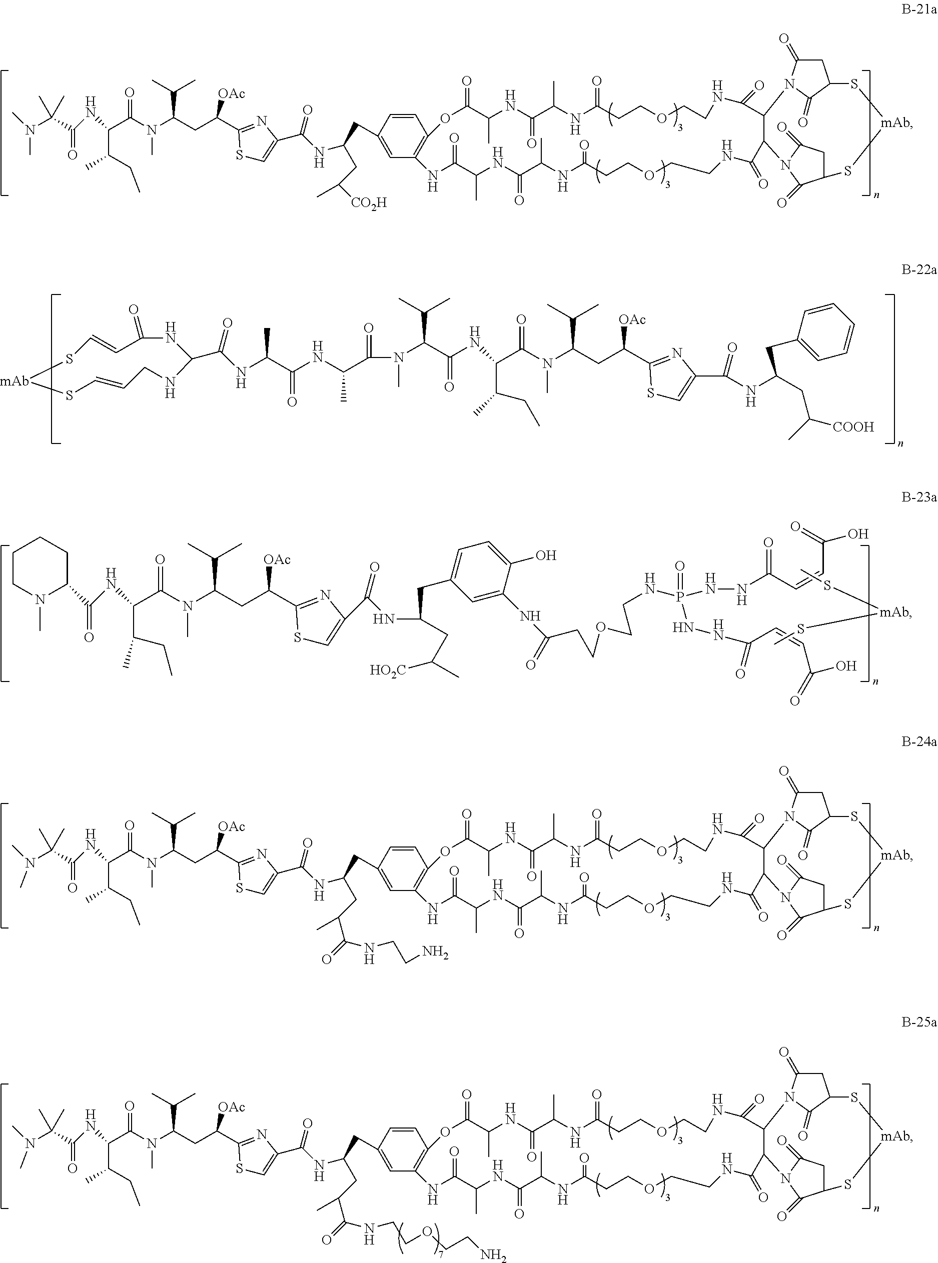

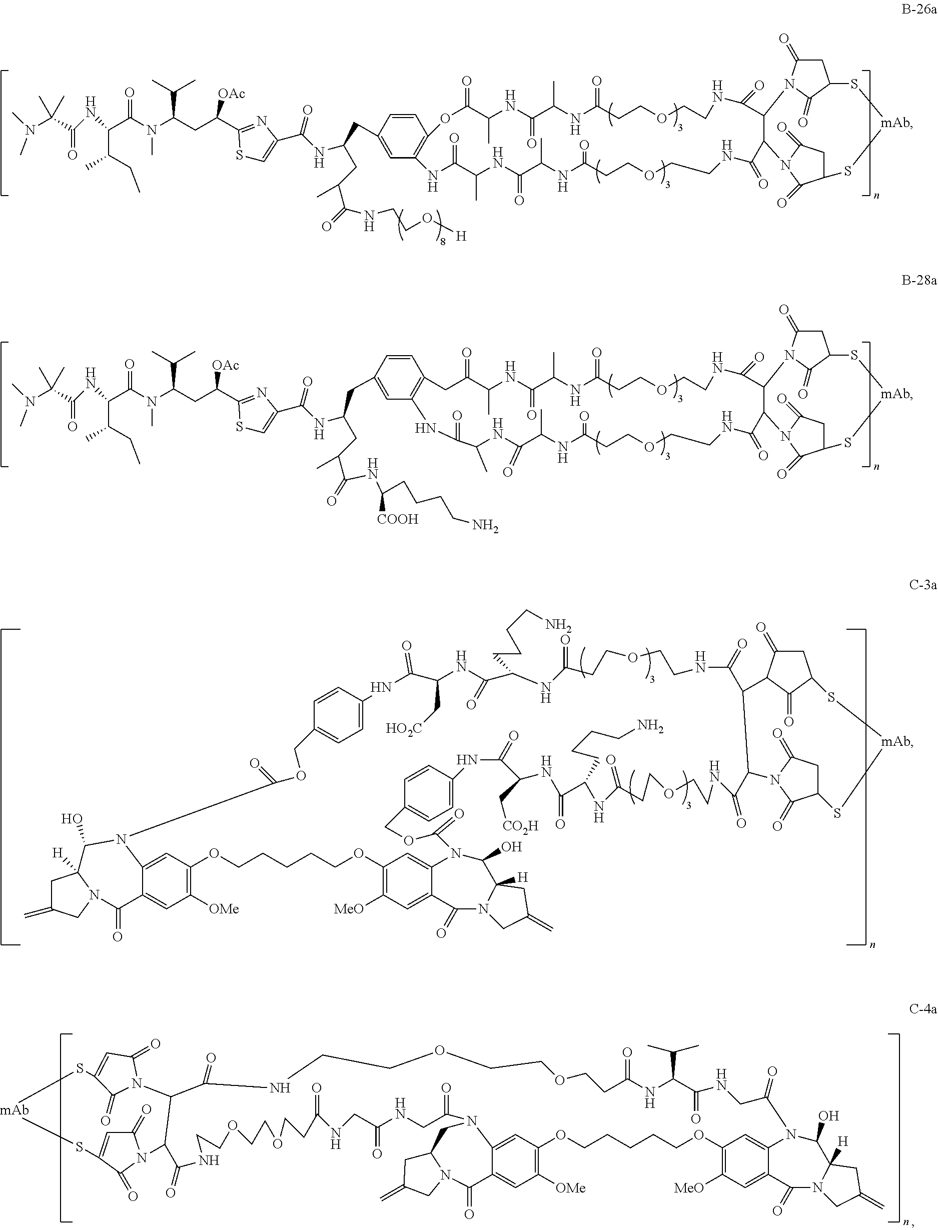

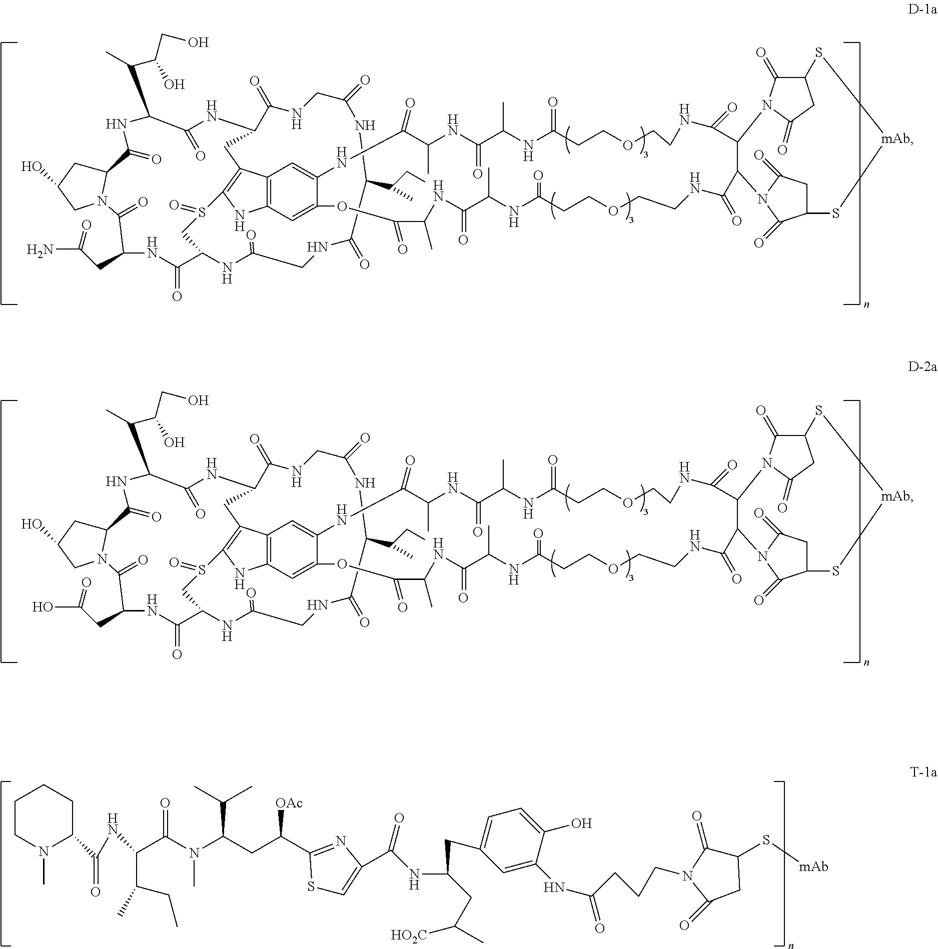

wherein n=2.0˜4.5

### Example 237. In Vitro Cytotoxicity Evaluation of Conjugate A-3a, A-4a, A-5a, B-3a, B-6a, B-9a, B-12a, B-15a, B-18a, B-19a, B-20a, B-21a, B-22a, B-23a, B-24, B-25, B-26, B-28, C-3a, C-4a, D-1a or D-2a in Comparison with T-DM1

The cell line used in the cytotoxicity assays was NCI-N87, a human gastric carcinoma cell line; The cells were grown in RPMI-1640 with 10% FBS. To run the assay, the cells (180 μl, 6000 cells) were added to each well in a 96-well plate and incubated for 24 hours at 37° C. with 5% CO₂. Next, the cells were treated with test compounds (20 μl) at various concentrations in appropriate cell culture medium (total volume, 0.2 mL). The control wells contain cells and the medium but lack the test compounds. The plates were incubated for 120 hours at 37° C. with 5% CO₂. MTT (5 mg/ml) was then added to the wells (20 μl) and the plates were incubated for 1.5 hr at 37° C. The medium was carefully removed and DMSO (180 μl) was added afterward. After it was shaken for 15 min, the absorbance was measured at 490 nm and 570 nm with a reference filter of 620 nm. The inhibition % was calculated according to the following equation:

inhibition %=[1−(assay-blank)/(control-blank)]×100.

The cytotoxicity results of IC₅₀:

| | DAR | N87 cell | N87 cell |
|---|---|---|---|
| | (drug | (Ag+) | (Ag+) |
| | ratio) | IC₅₀ (nM) | IC₉₀ (nM) |
| Conjugate A-3a | 3.5 | 0.32 nM | 0.91 nM |
| Conjugate A-4a | 3.8 | 0.17 nM | 0.87 nM |
| Conjugate A-5a | 4.1 | 0.094 nM | 0.31 nM |
| Conjugate B-3a | 3.8 | 0.14 nM | 0.28 |
| Conjugate B-6a | 3.8 | 0.21 nM | 0.62 |
| Conjugate B-9a | 3.6 | 0.17 nM | 0.67 |
| Conjugate B-12a | 3.8 | 0.13 nM | 0.06 |
| Conjugate B-15a | 3.6 | 0.29 nM | 0.92 |
| Conjugate B-18a | 3.6 | 0.46 nM | 1.20 |
| Conjugate B-19a | 3.5 | 0.12 nM | 0.63 |
| Conjugate B-20a | 3.8 | 0.33 nM | 0.96 |
| Conjugate B-21a | 3.8 | 0.42 nM | 1.10 |
| Conjugate B-22a | 3.6 | 0.13 nM | 0.33 |
| Conjugate B-23a | 3.6 | 0.18 nM | 0.38 |
| Conjugate B-24a | 3.8 | 0.83 nM | 1.46 |
| Conjugate B-25a | 3.8 | 0.72 nM | 1.82 |
| Conjugate B-26a | 3.7 | 0.93 nM | 1.93 |
| Conjugate B-28a | 3.6 | 0.45 nM | 0.78 |
| Conjugate C-3a | 3.6 | 0.09 nM | 0.17 |
| Conjugate C-4a | 3.7 | 0.26 nM | 0.48 |
| Conjugate D-1a | 3.8 | 0.041 nM | 0.087 |
| Conjugate D-2a | 3.9 | 0.033 nM | 0.072 |
| Conjugate T-1a | 3.8 | 0.25 nM | 0.51 |
| T-DM1 | 3.5 | 0.12 nM | 0.26 |

### Example 238. Antitumor Activity In Vivo (BALB/c Nude Mice Bearing NCI-N87 Xenograft Tumor)

The in vivo efficacy of conjugates A-3a, B-6a, B-12a, B-15a, B-18a, B-20a, B-21a, B-24a, B-28a, C-3a, and D-2a along with T-DM1 were evaluated in a human gastric carcinoma N-87 cell line tumor xenograft models. Five-week-old female BALB/c Nude mice (104 animals) were inoculated subcutaneously in the area under the right shoulder with N-87 carcinoma cells (5×10⁶ cells/mouse) in 0.1 mL of serum-free medium. The tumors were grown for 8 days to an average size of 110 mm³. The animals were then randomly divided into 13 groups (8 animals per group). The first group of mice served as the control group and was treated with the phosphate-buffered saline (PBS) vehicle. 10 groups were treated with conjugates A-3a, B-6a, B-12a, B-15a, B-18a, B-20a, B-21a, B-24a, B-28a, and T-DM1 respectively at dose of 3 mg/Kg administered intravenously. The remaining 2 groups were treated with conjugate C-3a and D-1a respectively at dose of 1 mg/Kg administered intravenously. Three dimensions of the tumor were measured every 4 days and the tumor volumes were calculated using the formula tumor volume=½ (length×width×height). The weight of the animals was also measured at the same time. A mouse was sacrificed when any one of the following criteria was met: (1) loss of body weight of more than 20% from pretreatment weight, (2) tumor volume larger than 2000 mm³, (3) too sick to reach food and water, or (4) skin necrosis. A mouse was considered to be tumor-free if no tumor was palpable.

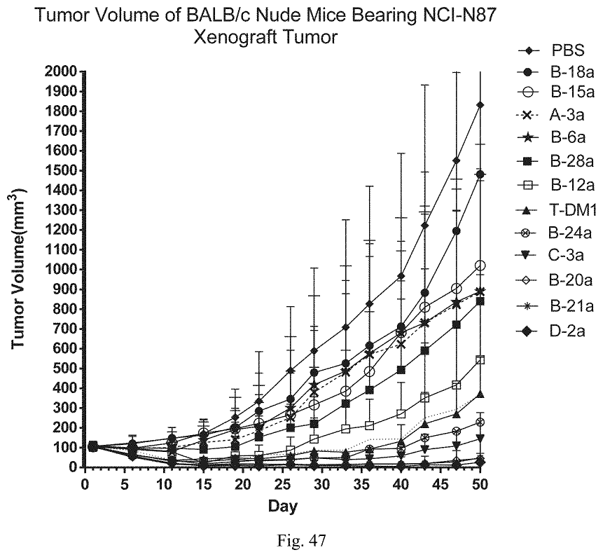

The results were plotted in FIG. 47. All the 13 conjugates did not cause the animal body weight loss. And the animals at control group were sacrificed at day 50 due to the tumor volume larger than 1800 mm³ and they were too sick. Here a1112 conjugates tested demonstrated anti-tumor activity. Animals at the groups of conjugate compounds B-24a, C-3a, B-20a, B-21a and D-20a demonstrated better anti-tumor activity than T-DM1. But the animals at the groups of conjugate compounds B-18a, B-15a, A-3a, B-6a, B-28a and B-12a showed worse anti-tumor activity than T-DM1. T-DM1 at dose of 3 mg/Kg inhibited the tumor growth for 28 days but it was not able to eliminate the tumors during the test. In contrast, conjugate compounds B-20a, B-21a, and D-20a eradicate some animal's tumors from day 15 until day 43. The inhibitions of the tumor growth at these doses are listed below:

| conjugate | Tumor growth delay | |
|---|---|---|
| T-DM1 | 28 | days |
| B-18a | 3 | days |
| B-15a | 5 | days |
| A-3a | 7 | days |
| B-6a | 8 | days |
| B-28a | 10 | days |
| B-12a | 19 | days |
| B-24a | 33 | days |
| C-3a | 39 | days |
| B-20a | >45 | days |
| B-21a | >45 | days |
| D-2a | >45 | days |

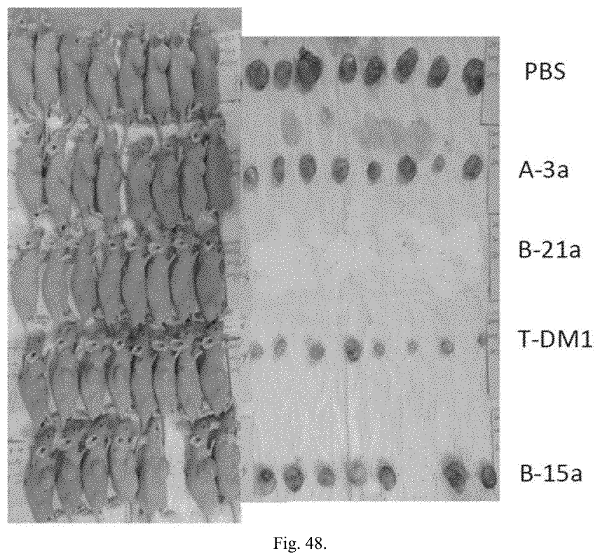

At the end of the experiment (day 50), animals of the group PBS, A-3a, B-21a, T-DM1 and B-15a were sacrificed and the tumors were stripped out and are shown in the picture of FIG. 48.

### Example 239. Stability Study of the Conjugate Having a Bis-Linkage in Comparison with Regular Conjugates Having a Mono-Linkage in the Mouse Serum

Forty-five female ICR mice, 6-7 weeks old, were separated into 3 groups. Each group included 15 mice for the PK study of one out of three ADCs. These 15 mice were further randomly divided into three groups (n=5). Each mouse was given conjugates T-DM₁, B-21a, and T-1a (Huang Y. et al, Med Chem. #44, 249^{th} ACS National Meeting, Denver, Colo., Mar. 22-26, 2015; WO2014009774) respectively at dose of 10 mg/Kg/per rat, i.v. bolus. The blood collection was followed the NCI's Guidelines for Rodent Blood Collection. Basically, mice in each group were taken turn for bleeding in order to avoid more than twice bleedings in a period of 24 hr. Blood was taken from retro-orbital blood sinus with a 70 uL capillary at time 0 (pre-dosing), 0.083, 0.25, 0.5, 1, 4, 8, 24, 48, 96, 168, 312 and 504 hrs post dosing. Plasma samples were analyzed for total antibodies and drug-conjugated antibodies by specific ELISA techniques. In brief, the conjugated antibody or the total antibody concentration in the mouse serum was measured as follows: 96-well ELISA plates were respectively coated overnight at 4° C. with anti-DM1 antibody, anti-tubulysin antibody or anti-Her-2's Fab antibody (1 ug/mL in 10 mM PBS, pH7.2). The plates were then washed three times with a washing buffer PBS-T (PBS/0.02% Tween20), and then blocked with a dilution buffer 1% (w/v) BSA/PBS-T for 1 hour at 37° C. After the blocking buffer was removed, the standards or mouse serum samples each with triple replicates were diluted in 1% BSA/PBS-T buffer, incubated at 37° C. for 1 hour, then the AP-conjugated donkey anti-human antibody was added for 30 minutes at 37° C. after the plates were washed. Plates were washed again, followed by the addition of pNPP substrate for the color development and then read on a microplate reader at 405 nm wavelength once the color development reaction was quenched with the 1 mol/L sodium hydroxide. The concentration of the conjugated antibody or the total antibody was obtained from a four-parameter curve fitting of the standard curve.

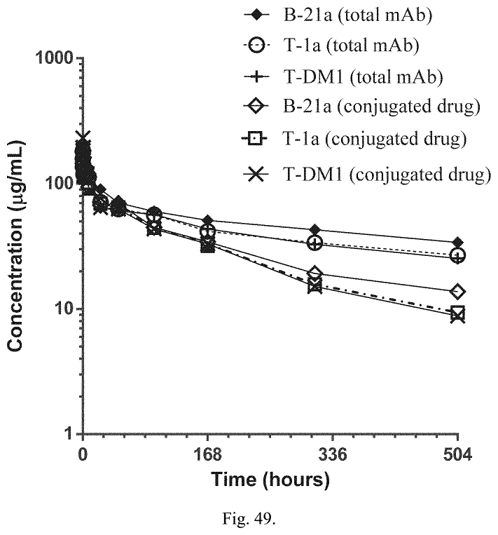

The result as shown in FIG. 49, the PK behaviors of total antibodies and drug-conjugated antibodies after dosing three ADCs presented as typical two-phase clearance curves. Equivalences between plasma and peripheral tissues were reached 8 hrs post-dosing. Elimination phase emerged 24 hr post-dosing and continued until the last sampling time point. In summary, the values of conjugate exposures (Aucₗₐₛₜ) for these three ADCs are 14981, 14713, and 16981 hr·ug/kg for T-DM1, T-1a and B-21a respectively. Distribution volumes for all these three conjugates are double of total blood volumes. The clearances (CL) of the conjugates are 0.59, 0.57, and 0.47 mL/hr/kg, which are almost halves of those for total antibodies. The clearance of B-21a, both conjugate and total antibodies, are smaller than those of other two ADCs, which indicates that the conjugate having the bis-linkage is more stable than the regular mono-linked conjugates in the mouse serum.

1. A bis-linkaged conjugate compound of Formula (I): 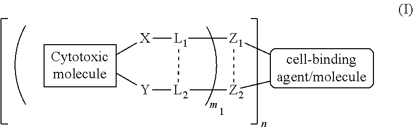
wherein
“ [symbol] ” represents a single bond; “ [symbol] ” is a single bond, a double bond, or absent; n and m₁ are 1 to 20 independently;
a cell-binding molecule that links to Z₁ and Z₂ is a molecule that binds to, complexes with, or reacts with a moiety of a target cell, the cell-binding molecule being an immunotherapeutic protein, an antibody, a single chain antibody; an antibody fragment that binds to the target cell; a monoclonal antibody; a single chain monoclonal antibody; a monoclonal antibody fragment that binds to the target cell; a chimeric antibody; a chimeric antibody fragment that binds to the target cell; a domain antibody; a domain antibody fragment that binds to the target cell; adnectins that mimic antibodies; DARPins; a lymphokine; a hormone; a vitamin; a growth factor; a colony stimulating factor; a nutrient-transport molecule; a transferrin; a binding peptide having at least four aminoacids; or an antibody, a protein, a small cell-binding molecule or a binding-ligand attached on albumin, polymers, dendrimers, liposomes, nanoparticles, vesicles, or on (viral) capsids;
a cytotoxic molecule is a therapeutic drug/molecule/agent, or an immunotherapeutic protein/molecule, or a function molecule for enhancement of binding or stabilization of the cell-binding molecule, or a cell-surface receptor binding ligand, or for inhibition of cell proliferation, or for monitoring, detection or study of a cell-binding molecule action; or an analog, or prodrug, or a pharmaceutically acceptable salt, hydrate, or hydrated salt, or a crystalline structure, or an optical isomer, racemate, diastereomer or enantiomer, of an immunotherapeutic compound, a chemotherapeutic compound, an antibody (probody) or an antibody (probody) fragment; or siRNA or DNA molecule; or a cell surface binding ligand; or an analog or prodrug of a therapeutic drug selected from the group consisting of tubulysins, calicheamicins, auristatins, maytansinoids, CC-1065 analogs, morpholinos doxorubicins, taxanes, cryptophycins, amatoxins, epothilones, eribulin, geldanamycins, duocarmycins, daunomycins, methotrexates, vindesines, vincristines, and benzodiazepine dimers (including dimers of pyrrolobenzodiazepine (PBD), tomaymycin, indolinobenzodiazepines, imidazobenzothiadiazepines, or oxazolidinobenzodiazepines);
X and Y represent the same or different, and independently, a functional group that links the cytotoxic molecule via a disulfide, thioether, thioester, peptide, hydrazone, ether, ester, carbamate, carbonate, amine (secondary, tertiary, or quartary), imine, cycloheteroalkyane, heteroaromatic, alkoxime or amide bond; X and Y are independently selected from the group consisting of NH; NHNH; N(R₁); N(R₁)N(R₂); O; S; S—S, O—NH, O—N(R₁), CH₂—NH, CH₂—N(R₁), CH═NH, CH═N(R₁), S(O), S(O₂), P(O)(OH), S(O)NH, S(O₂)NH, P(O)(OH)NH, NHS(O)NH, NHS(O₂)NH, NHP(O)(OH)NH, N(R₁)S(O)N(R₂), N(R₁)S(O₂)N(R₂), N(R₁)P(O)(OH)N(R₂), OS(O)NH, OS(O₂)NH, OP(O)(OH)NH, C(O), C(NH), C(NR₁), C(O)NH, C(NH)NH, C(NR₁)NH, OC(O)NH, OC(NH)NH; OC(NR₁)NH, NHC(O)NH; NHC(NH)NH; NHC(NR₁)NH, C(O)NH, C(NH)NH, C(NR₁)NH, OC(O)N(R₁), OC(NH)N(R₁), OC(NR₁)N(R₁), NHC(O)N(R₁), NHC(NH)N(R₁), NHC(NR₁)N(R₁), N(R₁)C(O)N(R₁), N(R₁)C(NH)N(R₁), N(R₁)C(NR₁)N(R₁); C₁-C₆ alkyl, C₂-C₈ alkenyl, heteroalkyl, alkylcycloalkyl, or heterocycloalkyl; and C₃-C₈ aryl, Ar-alkyl, heterocyclic, carbocyclic, cycloalkyl, heteroalkylcycloalkyl, alkylcarbonyl, or heteroaryl;
Z₁ and Z₂ are, the same or different, and independently a function group that is linked to the cell-binding molecule, to form a disulfide, ether, ester, thioether, thioester, peptide, hydrazone, carbamate, carbonate, amine (secondary, tertiary, or quarter), imine, cycloheteroalkyane, heteroaromatic, alkyloxime or amide bond; Z₁ and Z₂ independently are: C(O)CH, C(O)C, C(O)CH₂, ArCH₂, C(O), NH; NHNH; N(R₁); N(R₁)N(R₂); O; S; S—S, O—NH, O—N(R₁), CH₂—NH, CH₂—N(R₁), CH═NH, CH═N(R₁), S(O), S(O₂), P(O)(OH), S(O)NH, S(O₂)NH, P(O)(OH)NH, NHS(O)NH, NHS(O₂)NH, NHP(O)(OH)NH, N(R₁)S(O)N(R₂), N(R₁)S(O₂)N(R₂), N(R₁)P(O)(OH)N(R₂), OS(O)NH, OS(O₂)NH, OP(O)(OH)NH, C(O), C(NH), C(NR₁), C(O)NH, C(NH)NH, C(NR₁)NH, OC(O)NH, OC(NH)NH; OC(NR₁)NH, NHC(O)NH; NHC(NH)NH; NHC(NR₁)NH, C(O)NH, C(NH)NH, C(NR₁)NH, OC(O)N(R₁), OC(NH)N(R₁), OC(NR₁)N(R₁), NHC(O)N(R₁), NHC(NH)N(R₁), NHC(NR₁)N(R₁), N(R₁)C(O)N(R₁), N(R₁)C(NH)N(R₁), N(R₁)C(NR₁)N(R₁); C₁-C₈ alkyl, C₂-C₈ heteroalkyl, alkylcycloalkyl, or heterocycloalkyl; or C₃-C₈ aryl, Ar-alkyl, heterocyclic, carbocyclic, cycloalkyl, heteroalkylcycloalkyl, alkylcarbonyl, or heteroaryl;
L₁ and L₂ are, the same or different, independently selected from O; NH; S; NHNH; N(R₃); N(R₃)N(R_{3′}); C₁-C₈ alkyl, amide, amines, imines, hydrazines, or hydrazones; C₂-C₈ heteroalkyl, alkylcycloalkyl, ethers, esters, hydrazones, ureas, semicarbazides, carbazides, alkoxyamines, alkoxylamines, urethanes, amino acids, peptides, acyloxylamines, hydroxamic acids, or heterocycloalkyl; C₃-C₈ aryl, Ar-alkyl, heterocyclic, carbocyclic, cycloalkyl, heteroalkylcycloalkyl, alkylcarbonyl, or heteroaryl; 1˜8 amino acids; and polyethyleneoxy unit of formula (OCH₂CH₂)ₚOR₃, or (OCH₂CH(CH₃))ₚOR₃, or NH(CH₂CH₂O)ₚR₃, or NH(CH₂CH(CH₃)O)ₚR₃, or N[(CH₂CH₂O)ₚR₃]—[(CH₂CH₂O)_{p′}R_{3′}], or (OCH₂CH₂)ₚCOOR₃, or CH₂CH₂(OCH₂CH₂)ₚCOOR₃, wherein p and p′ are independently an integer selected from 0 to about 5000, or a combination thereof; R₃ and R₃, are independently H; C₁-C₈ alkyl; C₂-C₈ heteroalkyl, alkylcycloalkyl, or heterocycloalkyl; C₃-C₈ aryl, Ar-alkyl, heterocyclic, carbocyclic, cycloalkyl, heteroalkylcycloalkyl, alkylcarbonyl, or heteroaryl; or C₂-C₈ ester, ether, or amide; or 1˜8 amino acids; or polyethyleneoxy having formula (OCH₂CH₂)ₚ or (OCH₂—CH(CH₃))ₚ, wherein p is an integer from 0 to about 5000, or a combination thereof;
or L₁ and L₂ independently have one or more linker components of 6-maleimidocaproyl (“MC”), maleimidopropanoyl (“MP”), valine-citrulline (“val-cit” or “vc”), alanine-phenylalanine (“ala-phe” or “af”), p-aminobenzyloxycarbonyl (“PAB”), 4-thiopentanoate (“SPP”), 4-(N-maleimidomethyl)cyclohexane-1 carboxylate (“MCC”), (4-acetyl)amino-benzoate (“SIAB”), 4-thio-butyrate (SPDB), 4-thio-2-hydroxysulfonyl-butyrate (2-Sulfo-SPDB), or natural or unnatural peptide having 1˜8 natural or unnatural amino acid units;
or L₁ and L₂ independently contain a self-immolative component, peptidic unit, a hydrazone bond, a disulfide, an ester, an oxime, an amide, or a thioether bond; the self-immolative unit being an aromatic compound that is electronically similar to para-aminobenzyl-carbamoyl (PAB) groups, 2-aminoimidazol-5-methanol derivatives, heterocyclic PAB analogs, beta-glucuronide, and ortho or para-aminobenzylacetals; or one of following structures: 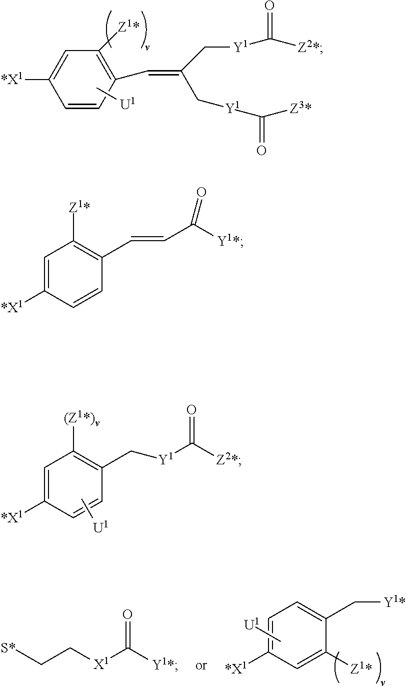
wherein the (*) atom is a point of attachment of additional spacer or releasable linker unit, or the cytotoxic molecule, and/or the cell-binding molecule; X¹, Y¹, Z² and Z³ are independently NH, O, or S; Z¹ is independently H, NHR₁, OR₁, SR₁, or COX₁R₁, wherein X₁ and R₁ are defined above; v is 0 or 1; U¹ is independently H, OH, C₁˜C₆ alkyl, (OCH₂CH₂)ₙ, F, Cl, Br, I, OR₅, SR₅, NR₅R₅′, N═NR₅, N═R₅, NR₅R₅′, NO₂, SOR₅R₅′, SO₂R₅, SO₃R₅, OSO₃R₅, PR₅R₅′, POR₅R₅′, PO₂R₅R₅′, OPO(OR₅)(OR₅′), or OCH₂PO(OR₅(OR₅′), wherein R₅ and R₅′ are independently selected from H, C₁˜C₈ alkyl; C₂˜C₈ alkenyl, alkynyl, heteroalkyl, or amino acid; C₃˜C₈ aryl, heterocyclic, carbocyclic, cycloalkyl, heterocycloalkyl, heteroaralkyl, alkylcarbonyl, or glycoside; or pharmaceutical cation salts;
or L₁ and L₂ independently have a non-self-immolative linker component containing one of following structures: 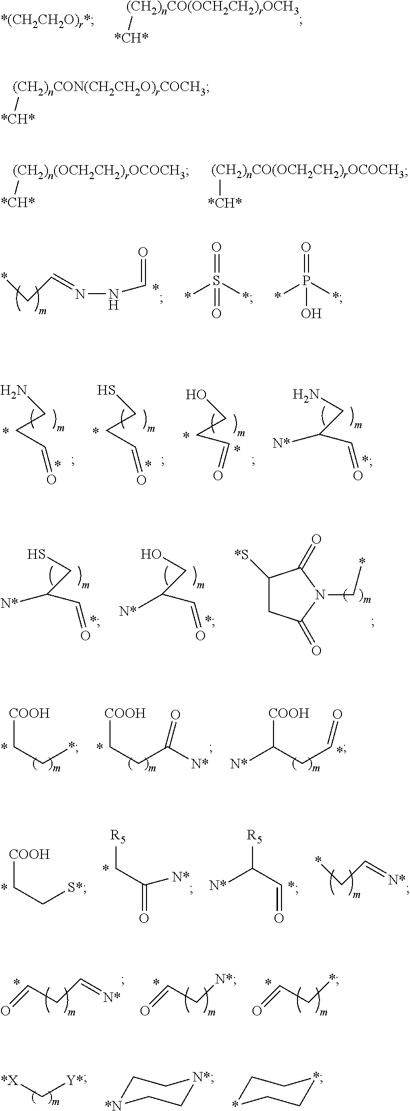 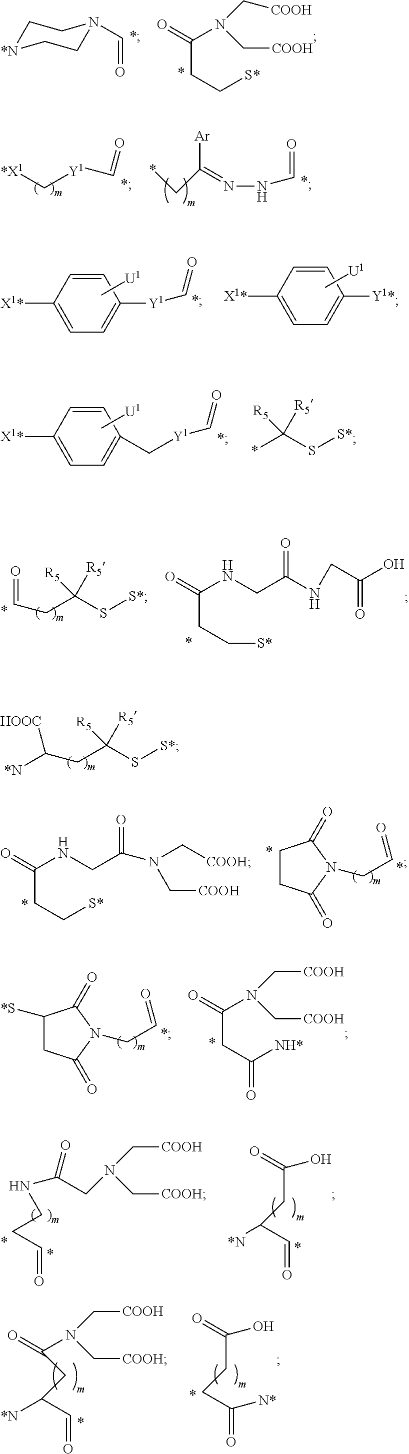 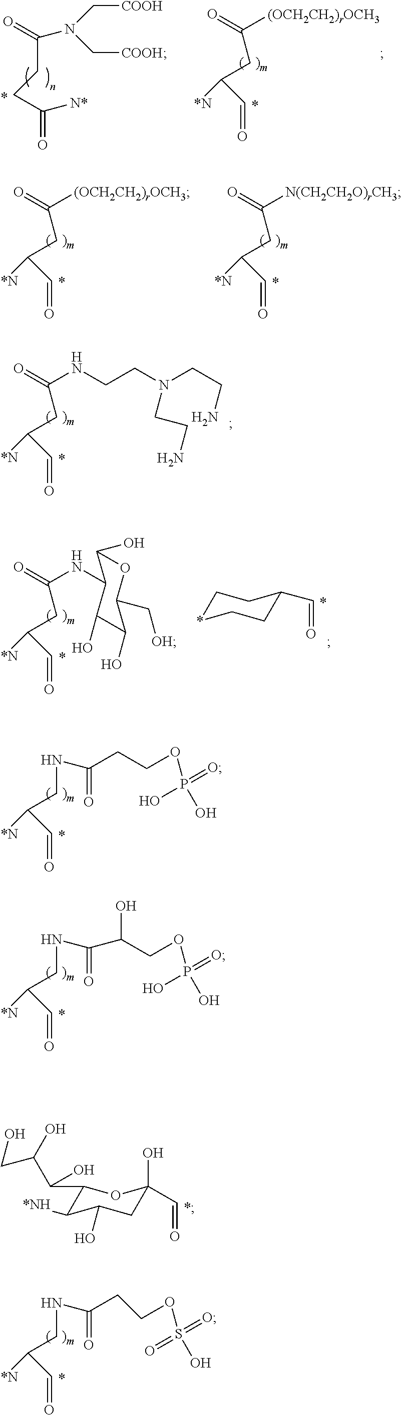 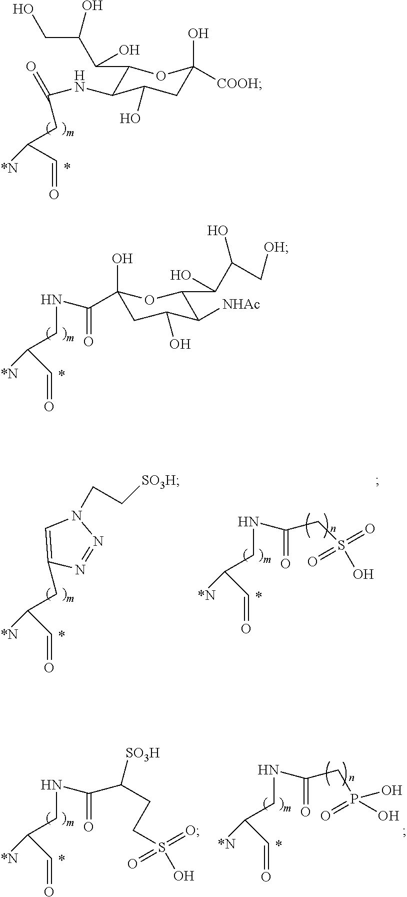
wherein the (*) atom is a point of attachment of additional spacer or releasable linker, the cytotoxic molecule, and/or the cell-binding molecule; X¹, Y¹, U¹, R₅, R₅′ are defined as above; r is 0˜100; m and n are 0˜6 independently;
or L₁ and L₂ independently are a releasable linker containing at least one bond that is capable of being broken under physiological conditions: a pH-labile, acid-labile, base-labile, oxidatively labile, metabolically labile, biochemically labile or enzyme-labile bond, having one of following structures:
—(CR₅R₆)ₘ(Aa)ᵣ(CR₇R₈)ₙ(OCH₂CH₂)ₜ—, —(CR₅R₆)ₘ(CR₇R₈)ₙ(Aa)ᵣ(OCH₂CH₂)ₜ—, - (Aa)ᵣ-(CR₅R₆)ₘ(CR₇R₈)ₙ(OCH₂CH₂)ₜ—, —(CR₅R₆)ₘ(CR₇R₈)ₙ(OCH₂CH₂)ᵣ(Aa)ₜ-, —(CR₅R₆)ₘ—(CR₇═CR₈)(CR₉R₁₀)ₙ(Aa)ₜ(OCH₂CH₂)ᵣ—, —(CR₅R₆)ₘ(N_{R} C O)(Aa)ₜ(CR₉R₁₀)ₙ—(OCH₂CH₂)ᵣ—, —(CR₅R₆)ₘ(Aa)ₜ(NR₁₁CO)(CR₉R₁₀)ₙ(OCH₂CH₂)ᵣ—, —(CR₅R₆)ₘ(OCO)(Aa)ₜ(CR₉R₁₀)ₙ(OCH₂CH₂)ᵣ—, —(CR₅R₆)ₘ(OCNR₇)(Aa)ₜ(CR₉R₁₀)ₙ(OCH₂CH₂)ᵣ—, —(CR₅R₆)ₘ(CO)(Aa)ₜ(CR₉R₁₀)ₙ(OCH₂CH₂)ᵣ—, —(CR₅R₆)ₘ(NR₁₁CO)(Aa)ₜ(CR₉R₁₀)ₙ(OCH₂CH₂)ᵣ—, —(CR₅R₆)ₘ—(OCO)(Aa)ₜ(CR₉R₁₀)ₙ—(OCH₂CH₂)ᵣ—, —(CR₅R₆)ₘ(OCNR₇)(Aa)ₜ(CR₉R₁₀)ₙ(OCH₂CH₂)ᵣ—, —(CR₅R₆)ₘ(CO)(Aa)ₜ(CR₉R₁₀)ₙ—(OCH₂CH₂)ᵣ—, —(CR₅R₆)ₘ-phenyl-CO(Aa)ₜ(CR₇R₈)ₙ, —(CR₅R₆)ₘ-furyl-CO(Aa)ₜ(CR₇R₈)ₙ—, —(CR₅R₆)ₘ-oxazolyl-CO(Aa)ₜ(CR₇R₈)ₙ—, —(CR₅R₆)ₘ-thiazolyl-CO(Aa)ₜ(CCR₇R₈)ₙ—, —(CR₅R₆)ₜ-thienyl-CO(CR₇R₈)ₙ—, —(CR₅R₆)ₜ-imidazolyl-CO—(CR₇R₈)ₙ—, —(CR₅R₆)ₜ-morpholino-CO(Aa)ₜ-(CR₇R₈)ₙ—, —(CR₅R₆)ₜpiperazino-CO(Aa)ₜ-(CR₇R₈)ₙ—, —(CR₅R₆)ₜ—N-methylpiperazin-CO(Aa)ₜ-(CR₇R₈)ₙ—, —(CR₅R₆)ₘ-(Aa)ₜphenyl-, —(CR₅R₆)ₘ-(Aa)ₜfuryl-, —(CR₅R₆)ₘ-oxazolyl(Aa)ₜ-, —(CR₅R₆)ₘ-thiazolyl(Aa)ₜ-, —(CR₅R₆)ₘ-thienyl-(Aa)ₜ-, —(CR₅R₆)ₘ-imidazolyl(Aa)ₜ-, —(CR₅R₆)ₘ-morpholino-(Aa)ₜ-, —(CR₅R₆)ₘ-piperazino-(Aa)ₜ-, —(CR₅R₆)ₘ—N-methylpiperazino-(Aa)ₜ-, —K(CR₅R₆)ₘ(Aa)ᵣ(CR₇R₈)ₙ(OCH₂CH₂)ₜ—, —K(CR₅R₆)ₘ(CR₇R₈)ₙ(Aa)ᵣ(OCH₂CH₂)ₜ—, —K(Aa)ᵣ-(CR₅R₆)ₘ(CR₇R₈)ₙ(OCH₂CH₂)ₜ—, —K(CR₅R₆)ₘ(CR₇R₈)ₙ(OCH₂CH₂CH)ᵣ(Aa)ₜ-, —K(CR₅R₆)ₘ—(CR₇═CR₈)(CR₉R₁₀)ₙ(Aa)ₜ(OCH₂CH₂)ᵣ—, —K(CR₅R₆)ₘ(NR₁₁CO)(Aa)ₜ(CR₉R₁₀)ₙ(OCH₂CH₂)ᵣ—, —K(CR₅R₆)ₘ(Aa)ₜ(NR₁₁CO)(CR₉R₁₀)ₙ(OCH₂CH₂)ᵣ—, —K(CR₅R₆)ₘ(OCO)(Aa)ₜ(CR₉R₁₀)ₙ—(OCH₂CH₂)ᵣ—, —K(CR₅R₆)ₘ(OCNR₇)(Aa)ₜ(CR₉R₁₀)ₙ(OCH₂CH₂)ᵣ—, —K(CR₅R₆)ₘ(CO)(Aa)ₜ-(CR₉R₁₀)ₙ(OCH₂CH₂)ᵣ—, —K(CR₅R₆)ₘ(NR₁₁CO)(Aa)ₜ(CR₉R₁₀)ₙ(OCH₂CH₂)ᵣ—, —K(CR₅R₆)ₘ—(OCO)(Aa)ₜ(CR₉R₁₀)ₙ(OCH₂CH₂)ᵣ—, —K(CR₅R₆)ₘ(OCNR₇)(Aa)ₜ(CR₉R₁₀)ₙ(OCH₂CH₂)ᵣ—, —K—(CR₅R₆)ₘ(CO)(Aa)ₜ(CR₉R₁₀)ₙ(OCH₂CH₂)ᵣ—, —K(CR₅R₆)ₘ-phenyl-CO(Aa)ₜ(CR₇R₈)ₙ—, —K—(CR₅R₆)ₘ-furyl-CO(Aa)ₜ(CR₇R₈)ₙ—, —K(CR₅R₆)ₘ-oxazolyl-CO(Aa)ₜ(CR₇R₈)ₙ—, —K(CR₅R₆)ₘ-thiazolyl-CO(Aa)ₜ-(CR₇R₈)ₙ—, —K(CR₅R₆)ₜ-thienyl-CO(CR₇R₈)ₙ—, —K(CR₅R₆)ₜimidazolyl-CO—(CR₇R₈)—, —K(CR₅R₆)ₜmorpholino-CO(Aa)ₜ(CR₇R₈)ₙ—, —K(CR₅R₆)ₜpiperazino-CO(Aa)ₜ-(CR₇R₈)ₙ—, —K(CR₅R₆)ₜ—N-methylpiperazinCO(Aa)ₜ(CR₇R₈)ₙ—, —K(CR₅R₆)ₘ(Aa)ₜphenyl, —K—(CR₅R₆)ₘ-(Aa)ₜfuryl-, —K(CR₅R₆)ₘ-oxazolyl(Aa)ₜ-, —K(CR₅R₆)ₘ-thiazolyl(Aa)ₜ-, —K(CR₅R₆)ₘ-thienyl-(Aa)ₜ-, —K(CR₅R₆)ₘ-imidazolyl(Aa)ₜ-, —K(CR₅R₆)ₘ-morpholino(Aa)ₜ-, —K(CR₅R₆)ₘ-piperazino-(Aa)ₜG, —K(CR₅R₆)ₘN-methylpiperazino(Aa)ₜ-; wherein m, Aa, m, and n are described above; t and r are 0-100 independently; R₃, R₄, R₅, R₆, R₇, and R₈ are independently H; halide; C₁˜C₈ alkyl; C₂˜C₈ aryl, alkenyl, alkynyl, ether, ester, amine or amide, which optionally substituted by one or more halide, CN, NR₁R₂, CF₃, OR₁, Aryl, heterocycle, S(O)R₁, SO₂R₁, —CO₂H, —SO₃H, —OR₁, —CO₂R₁, —CONR₁, —PO₂R₁R₂, —PO₃H or P(O)R₁R₂R₃; K is NR₁, —SS—, —C(═O)—, —C(═O)NH—, —C(═O)O—, —C═NH—O—, —C═N—NH—, —C(═O)NH—NH—, O, S, Se, B, Het (heterocyclic or heteroaromatic ring having C₃-C₈), or peptide containing 1-20 amino acids;
or L₁ and L₂ independently contain one of following hydrophilic structures: 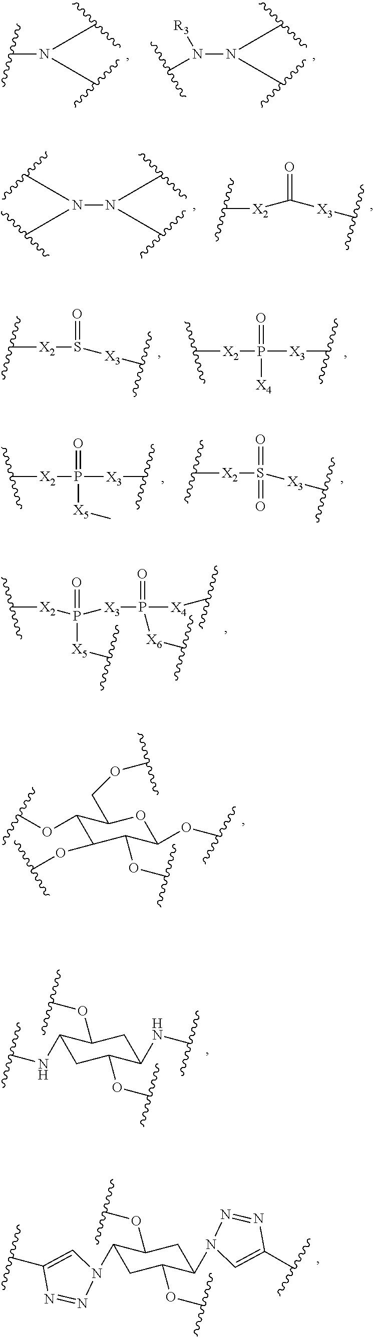 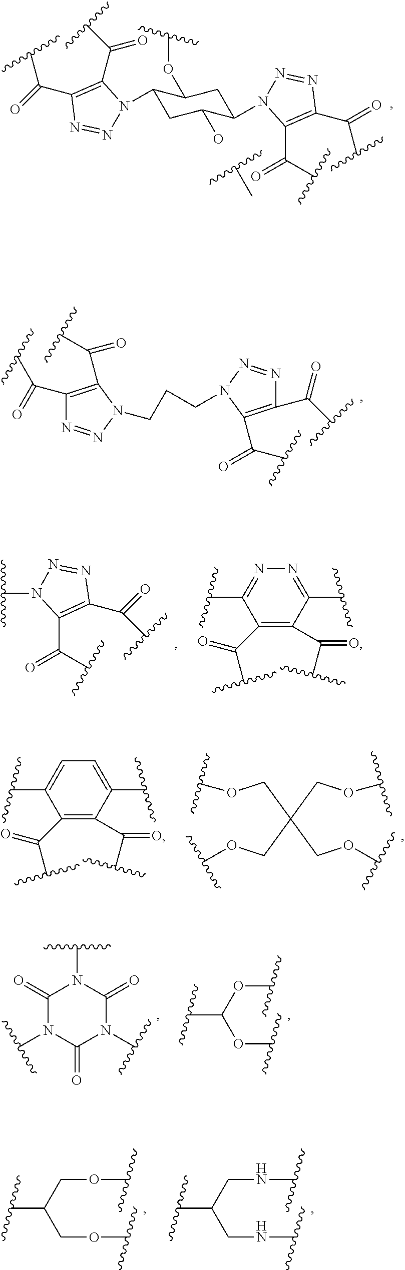 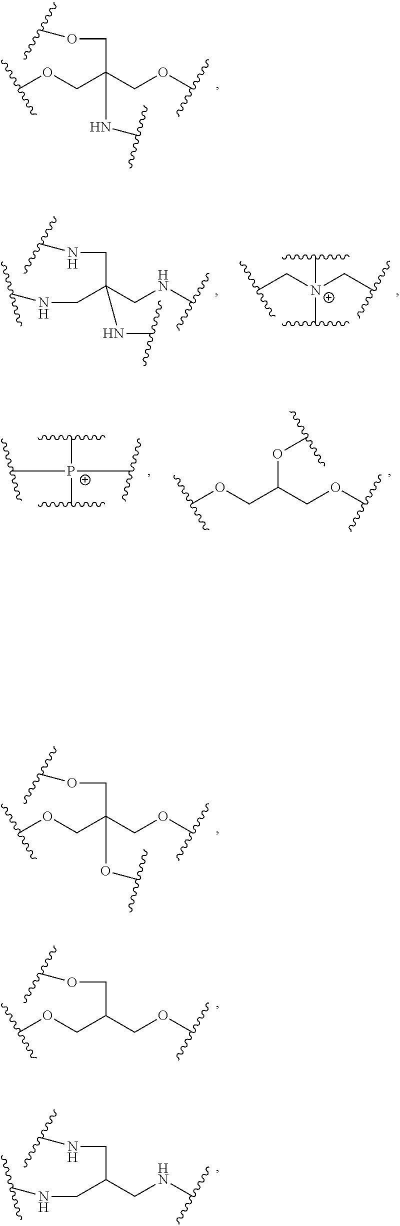 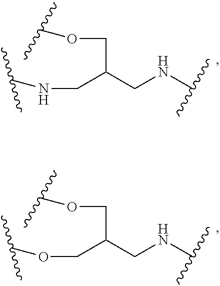
wherein [symbol] is a site of linkage; X₂, X₃, X₄, X₅, or X₆ are independently NH; NHNH; N(R₃); N(R₃)N(R_{3′}); O; S; C₁-C₆ alkyl; C₂-C₆ heteroalkyl, alkylcycloalkyl, or heterocycloalkyl; C₃-C₈ aryl, Ar-alkyl, heterocyclic, carbocyclic, cycloalkyl, heteroalkylcycloalkyl, alkylcarbonyl, or heteroaryl; or 1˜8 amino acids; wherein R₃ and R_{3′} are independently H; C₁-C₈ alkyl; C₂-C₈ hetero-alkyl, alkylcycloalkyl, or heterocycloalkyl; C₃-C₈ aryl, Ar-alkyl, heterocyclic, carbocyclic, cycloalkyl, heteroalkylcycloalkyl, alkylcarbonyl, or heteroaryl; or C₂-C₈ ester, ether, or amide; or polyethyleneoxy unit having formula (OCH₂CH₂)ₚ or (OCH₂CH(CH₃))ₚ, wherein p is an integer from 0 to about 5000, or a combination thereof;
or, X, Y, L₁, L₂, Z₁ or Z₂ are independently composed of one or more following components: 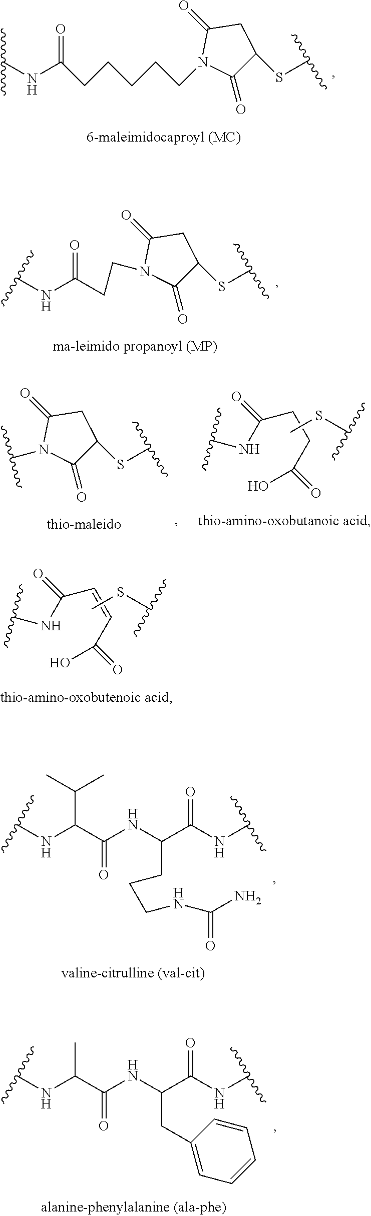 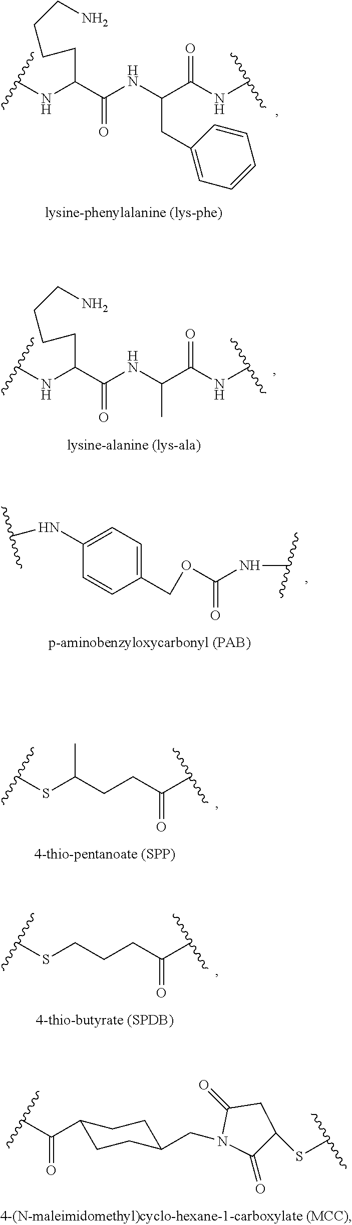 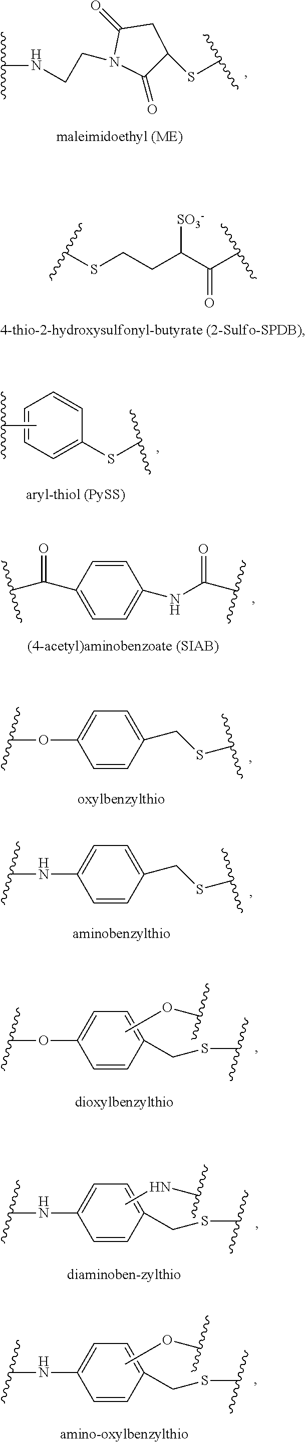 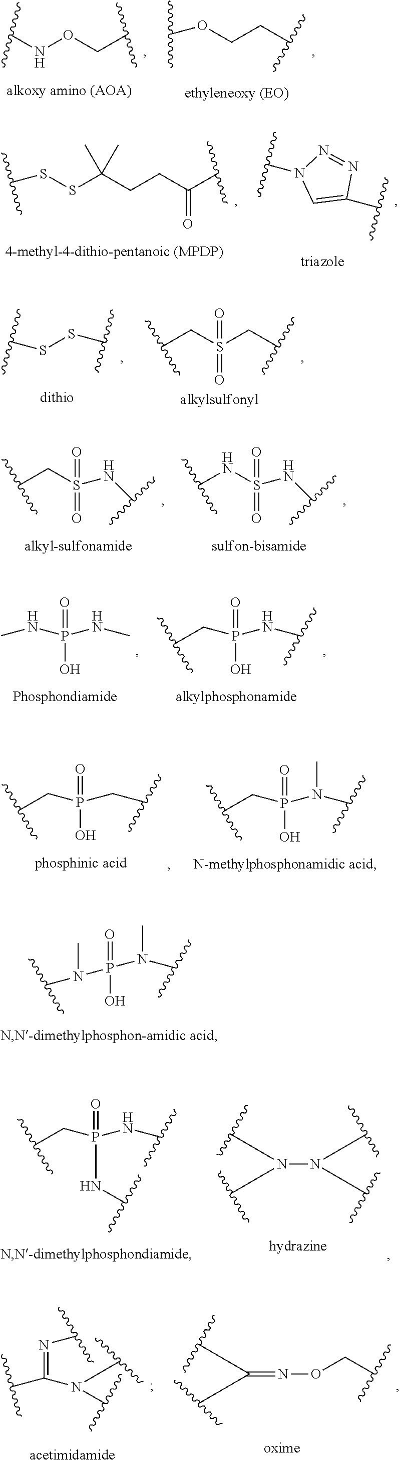 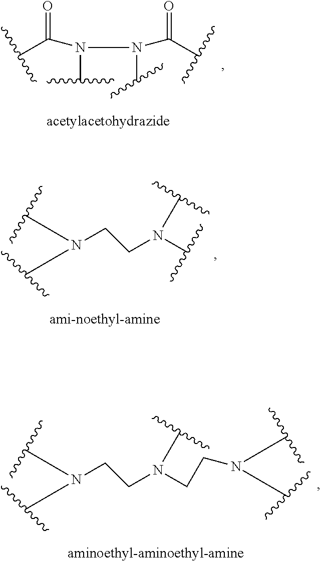 [SMILES: *C(=O)N(*)N(*)C(*)=O ; *N(*)CCN(*)* ; *N(*)CCN(*)CCN(*)*]
and L- or D-, natural or unnatural peptides containing 1-20 amino acids;
wherein [symbol] is a site that another bond is connected to;
or X, Y, L₁, L₂, Z₁, or Z₂, are independently absent, provided that L₁ and Z₁, or L₂ and Z₂ are not absent at the same time;
wherein the conjugate compound of Formula (I) excludes following structure: 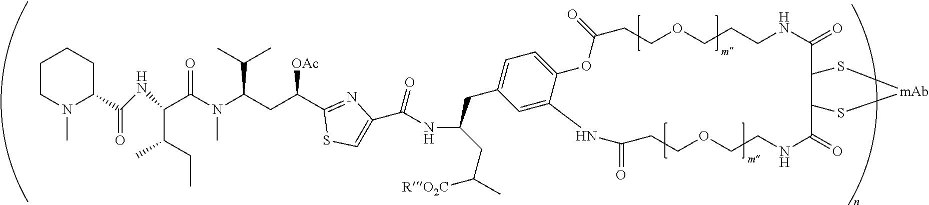
wherein n=1-30; m″=1-3; R′″═H, CH₃ or C₂H_{5.}

2. A bis-linker compound containing a cytotoxic molecule of Formula (II): 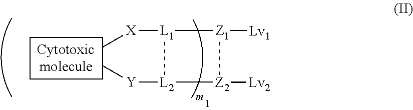
wherein:
“ [symbol] ” represents a single bond; “ [symbol] ” is a single bond, a double bond, a triple bond, or absent;
provided that when ----- represents a triple bond, both Lv₁ and Lv₂ are absent;
a cytotoxic molecule is a therapeutic drug/molecule/agent, or an immunotherapeutic protein/molecule, or a function molecule for enhancement of binding or stabilization of a cell-binding molecule, or a cell-surface receptor binding ligand, or for inhibition of cell proliferation, or for monitoring, detection or study of a cell-binding molecule action, or an analog, or prodrug, or a pharmaceutically acceptable salt, hydrate, or hydrated salt or a crystalline structure, or an optical isomer, racemate, diastereomer or enantiomer, of an immunotherapeutic compound, a chemotherapeutic compound, an antibody (probody) or an antibody (probody) fragment; or siRNA or DNA molecule; or a cell surface binding ligand; or an analog or prodrug of a therapeutic drug selected from the group consisting of tubulysins, calicheamicins, auristatins, maytansinoids, CC-1065 analogs, morpholinos doxorubicins, taxanes, cryptophycins, amatoxins, epothilones, eribulin, geldanamycins, duocarmycins, daunomycins, methotrexates, vindesines, vincristines, and benzodiazepine dimers (including dimers of pyrrolobenzodiazepine (PBD), tomaymycin, indolinobenzodiazepines, imidazobenzothiadiazepines, or oxazolidinobenzodiazepines);
m₁ is 1 to 20;
X and Y represent the same or different, and independently, a functional group that links the cytotoxic molecule via a disulfide, thioether, thioester, peptide, hydrazone, ether, ester, carbamate, carbonate, amine (secondary, tertiary, or quartary), imine, cycloheteroalkyane, heteroaromatic, alkoxime or amide bond; X and Y are independently selected from the group consisting of NH; NHNH; N(R₁); N(R₁)N(R₂); O; S; S—S; O—NH, O—N(R₁), CH₂—NH, CH₂—N(R₁), CH═NH, CH═N(R₁), S(O), S(O₂), P(O)(OH), S(O)NH, S(O)NH, P(O)(OH)NH, NHS(O)NH, NHS(O₂)NH, NHP(O)(OH)NH, N(R₁)S(O)N(R₂), N(R₁)S(O₂)N(R₂), N(R₁)P(O)(OH)N(R₂), OS(O)NH, OS(O₂)NH, OP(O)(OH)NH, C(O), C(NH), C(NR₁), C(O)NH, C(NH)NH, C(NR₁)NH, OC(O)NH, OC(NH)NH; OC(NR₁)NH, NHC(O)NH; NHC(NH)NH; NHC(NR₁)NH, C(O)NH, C(NH)NH, C(NR₁)NH, OC(O)N(R₁), OC(NH)N(R₁), OC(NR₁)N(R₁), NHC(O)N(R₁), NHC(NH)N(R₁), NHC(NR₁)N(R₁), N(R₁)C(O)N(R₁), N(R₁)C(NH)N(R₁), N(R₁)C(NR₁)N(R₁); C₁-C₆ alkyl, C₂-C₈ alkenyl, heteroalkyl, alkylcycloalkyl, or heterocycloalkyl; and C₃-C₈ aryl, Ar-alkyl, heterocyclic, carbocyclic, cycloalkyl, heteroalkylcycloalkyl, alkylcarbonyl, or heteroaryl;
Z₁ and Z₂ are, the same or different, and independently are: C(O)CH, C(O)C, C(O)CH₂, ArCH₂, C(O), NH; NHNH, N(R₁); N(R₁)N(R₂); O, S; S—S, O—NH, O—N(R₁), CH₂—NH, CH₂—N(R₁), CH═NH, CH—N(R₁), S(O), S(O₂), P(O)(OH), S(O)NH, S(O₂)NH, P(O)(OH)NH, NHS(O)NH, NHS(O₂)NH, NHP(O)(OH)NH, N(R₁)S(O)N(R₂), N(R₁)S(O₂)N(R₂), N(R₁)P(O)(OH)N(R₂), OS(O)NH, OS(O₂)NH, OP(O)(OH)NH, C(O), C(NH), C(NR₁), C(O)NH, C(NH)NH, C(NR₁)NH, OC(O)NH, OC(NH)NH; OC(NR₁)NH, NHC(O)NH; NHC(NH)NH, NHC(NR₁)NH, C(O)NH, C(NH)NH, C(NR₁)NH, OC(O)N(R₁), OC(NH)N(R₁), OC(NH)N(R₁), OC(NR₁)N(R₁)m NHC(O)N(R₁), NHC(NH)N(R₁), NHC(NR₁)N(R₁), N(R₁)C(O)N(R₁), N(R₁)C(NH)N(R₁), N(R₁)C(NR₁)N(R₁); C₁-C₈ alkyl, C₂-C₈ heteroalkyl, alkylcycloalkyl, or heterocycloalkyl; or C₃-C₈ aryl, Ar-alkyl, heterocyclic, carbocyclic, cycloalkyl, heteroalkylcycloalkyl, alkylcarbonyl, or heteroaryl;
L₁ and L₂ are, the same or different, independently selected from O; NH; S; NHNH; N(R₃); N(R₃)N(R_{3′}); C₁-C₈ alkyl, amide, amines, imines, hydrazines, or hydrazones; C₂-C₈ heteroalkyl, alkylcycloalkyl, ethers, esters, hydrazones, ureas, semicarbazides, carbazides, alkoxyamines, alkoxylamines, urethanes, amino acids, peptides, acyloxylamines, hydroxamic acids, or heterocycloalkyl, C₃-C₈ aryl, Ar-alkyl, heterocyclic, carbocyclic, cycloalkyl, heteroalkylcycloalkyl, alkylcarbonyl, or heteroaryl, 1˜8 amino acids; and polyethyleneoxy unit of formula (OCH₂CH₂)ₚOR₃, or (OCH₂CH(CH₃))ₚOR₃, or NH(CH₂CH₂))ₚR₃ or NH(CH₂CH(CH₃)O)ₚR₃, or N[(CH₂CH₂O)R₃]—[(CH₂CH₂O)_{p′}R_{3′}], or (OCH₂CH₂)ₚCOOR₃, or CH₂CH₂(OCH₂CH₂)ₚCOOR₃, wherein p and p′ are independently an integer selected from 0 to about 5000, or a combination thereof; R₃ and R_{3′} are independently H; C₁-C₈ alkyl, C₂-C₈ heteroalkyl, alkylcycloalkyl, or heterocycloalkyl; C₃-C₈ aryl, Ar-alkyl, heterocyclic, carbocyclic, cycloalkyl, heteroalkylcycloalkyl, alkylcarbonyl, or heteroaryl; or C₂-C₈ ester, ether, or amide; or 1˜8 amino acids; or polyethyleneoxy having formula (OCH₂CH₂)ₚ or (OCH₂—CH(CH₃))ₚ, wherein p is an integer from 0 to about 5000, or a combination thereof;
or L₁ and L₂ independently have one or more linker components of 6-maleimidocaproyl (“MC”), maleimidopropanoyl (“MP”), valine-citrulline (“val-cit” or “vc”), alanine-phenylalanine (“ala-phe” or “af”), p-aminobenzyloxycarbonyl (“PAB”), 4-thiopentanoate, “SPP”), 4-(N-maleimidomethyl)cyclohexane-1 carboxylate (“MCC”), 4-acetyl)amino-benzoate (“SIAB”), 4-thio-butyrate (SPDB), 4-thio-2-hydroxysulfonyl-butyrate (2-Sulfo-SPDB), or natural or unnatural peptide having 1˜8 natural or unnatural amino acid units;
or L₁ and L₂ independently contain a self-immolative component, peptidic unit, a hydrazone bond, a disulfide, an ester, an oxime, an amide, or a thioether bond, the self-immolative unit being an aromatic compound that is electronically similar to para-aminobenzyl-carbamoyl (PAB) groups, 2-aminoimidazol-5-methanol derivatives, heterocyclic PAB analogs, beta-glucuronide, and ortho or para-aminobenzylacetals, or one of following structures, 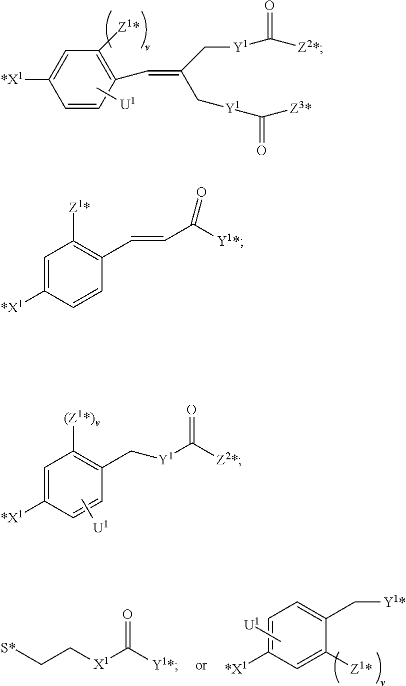
wherein the (*) atom is a point of attachment of additional spacer or releasable linker units, or the cytotoxic molecule, X¹, Y¹, Z² and Z³ are independently NH, O, or S; Z¹ is independently H, NHR₁, OR₁, SR₁, or COX₁R₁, wherein X₁ and R₁ are defined above; v is 0 or 1; U¹ is independently H, OH, C₁˜C₆ alkyl, (OCH₂CH₂)ₙ, F, Cl, Br, I, OR₅, SR₅, NR₅R₅′, N═NR₅, N═R₅, NR₅R₅′, NO₂, SOR₅R₅′, SO₂R₅, SO₃R₅, OSO₃R₅, PR₅R₅′, POR₅R₅′, PO₂R₅R₅′, OPO(OR₅)(OR₅′), or OCH₂PO(OR₅′), wherein R₅ and R₅′ are independently selected from H, C₁˜C₈ alkyl; C₂˜C₈ alkenyl, alkynyl, heteroalkyl, or amino acid; C₃˜C₈ aryl, heterocyclic, carbocyclic, cycloalkyl, heterocycloalkyl, heteroaralkyl, alkylcarbonyl, or glucoside; or pharmaceutical cation salts;
or L₁ and L₂ independently have n non-self-immolative linker component containing one of following structures 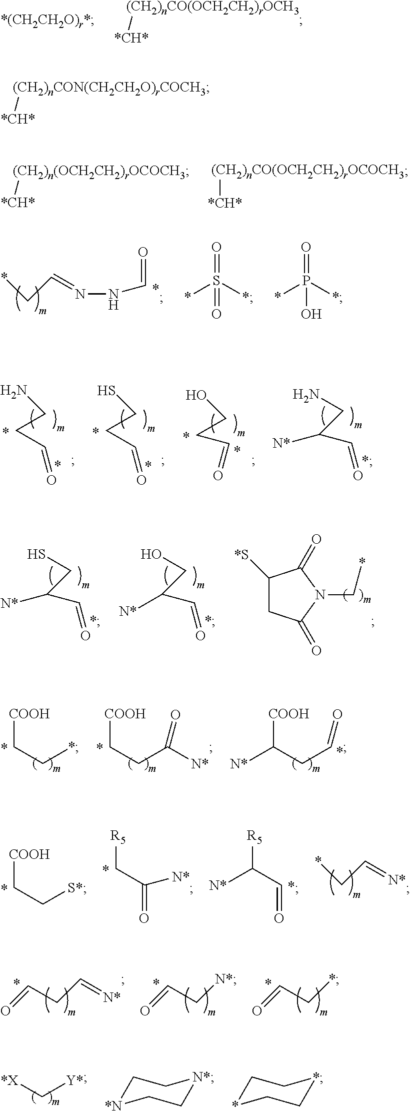 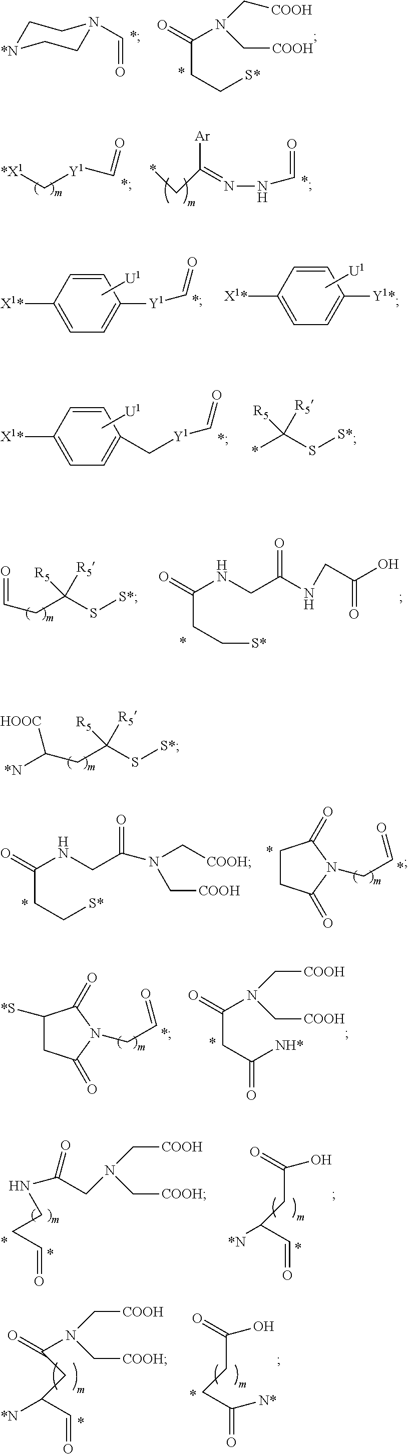 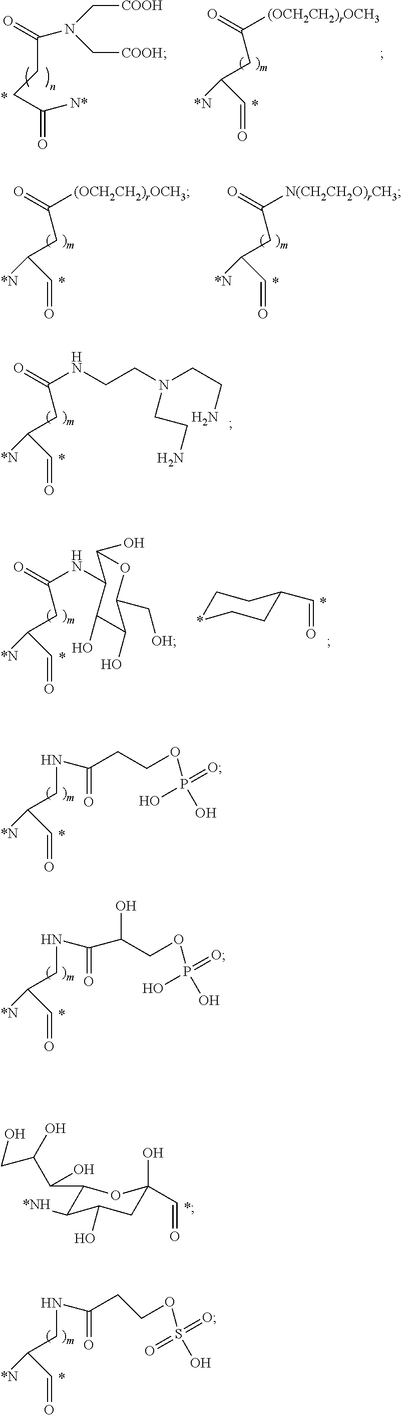 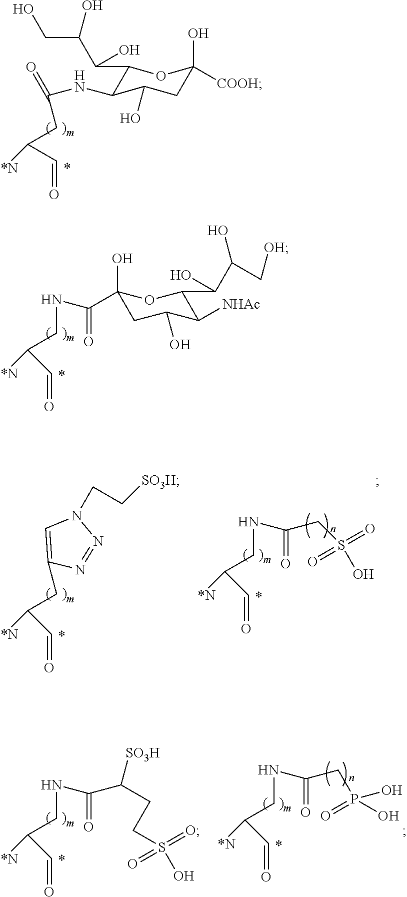
wherein the (*) atom is a point of attachment of additional spacer or releasable linker, or the cytotoxic molecule; X¹, Y¹, U¹, R₅, R₅′ are defined as above, r is 0˜100; m and n are 0˜6 independently;
or L₁ and L₂ independently are a releasable linker containing at least one bond that is capable of being broken under physiological conditions; a pH-labile, acid-labile, base-labile, oxidatively labile, metabolically labile, biochemically labile or enzyme-labile bond, having one of following structures:
—(CR₅R₆)ₘ(Aa)r(CR₇R₈)ₙ(OCH₂CH₂)ᵣ—, —(CR₅R₆)ₘ(CR₇R₈)ₙ(Aa)ᵣ(OCH₂CH₂)ᵣ—, -(Aa)ᵣ-(CR₅R₆)ₘ(CR₇R₈)ₙ(OCH₂CH₂)ᵣ—, —(CR₅R₆)ₘ(CR₇R₈)ₙ(OCH₂CH₂)ᵣ(Aa)ᵣ, —(CR₅R₆)ₘ(CR₇═CR₈)(CR₉R₁₀)ₙ(Aa)ₜ(OCH₂CH₂)ᵣ, —CR₅R₆)ₘ(NR₁₁CO)(Aa)ₜ(CR₉R₁₀)ₙ(OCH₂CH₂)ᵣ, —(CR₅R₆)ₘ(Aa)ₜ(NR₁₁CO)(CR₉R₁₀)ₙ(OCH₂CH₂)r-, —(CR₅R₆)ₘ(OCO)(Aa)ₜ(CR₉R₁₀)ₙ—(OCH₂CH₂)ᵣ—, —(CR₅R₆)ₘ(OCNR₇)(Aa)ₜ(CR₉R₁₀)ₙ(OCH₂CH₂)ᵣ—, —(CR₅R₆)ₘ(CO)(Aa)t(CR₉R₁₀)ₙ(OCH₂CH₂)ᵣ—, —(CR₅R₆)ₘ(NR₁₁CO)(Aa)ₜ(CR₉R₁₀)ₙ(OCH₂CH₂)ᵣ—, —(CR₅R₆)m-)OCO)(Aa)t(CR₉R₁₀)ₙ—(OCH₂CH₂)ₜ—, —(CR₅R₆)ₘ(OCNR₇)(Aa)t(CR₉R₁₀)ₙ(OCH₂CH₂)ᵣ—, —(CR₅R₆)ₘ(CO)(Aa)ₜ(CR₉R₁₀)ₙ(OCH₂CH₂)ᵣ—, —(CR₅R₆)ₘ-phenyl-CO(Aa)ₜ(CR₇R₈)ₙ—, —(CR₅R₆)ₘ-furyl-CO(Aa)ₜ(CR₇R₈)ₙ—, —(CR₅R₆)ₘ-oxazolyl-CO(Aa)ₜ(CR₇R₈)ₙ—, —(CR₅R₆)ₘ-thiazolyl-CO(Aa)ₜ(CCR₇R₈)ₙ—, —(CR₅R₆)ₜ-thienyl-CO(CR₇R₈)ₙ—, —(CR₅R₆)ₜ-imidazolyl-CO—(CR₇R₈)ₙ—, —(CR₅R₆)ₜ-morpholino-CO(Aa)ₜ-(CR₇R₈)ₙ—, —(CR₅R₆)ₜpiperazino-CO(Aa)t-(CR₇R₈)ₙ—, —(CR₅R₆)ₜ—N-methylpiperazin-CO(Aa)ₜ-(CR₇R₈)ₙ—, —(CR₅R₆)ₘ-(Aa)ₜphenyl-, —(CR₅R₆)ₘ-(Aa)ₜfuryl-, —(CR₅R₆)ₘ-oxazolyl(Aa)ₜ-, —(CR₅R₆)ₘ-thiazolyl(Aa)ₜ-, —(CR₅R₆)ₘ-thienyl-(Aa)ₜ-, —(CR₅R₆)ₘ-imidazoyl(Aa)ᵣ-, —(CR₅R₆)ₘ-morpholino-(Aa)ₜ-, —(CR₅R₆)ₘ-piperazino-(Aa)ₜ-, —(CR₅R₆)ₘ—N-methylpiperazino-(Aa)ₜ-, —K(CR₅R₆)ₘ(Aa)r(CR₇R₈)ₙ(OCH₂CH₂)t-, —K(CR₅R₆)ₘ(CR₇R₈)ₙ(Aa)r(OCH₂CH₂)ₜ—, —K(Aa)ₜ-(CR₅R₆)ₘ(CR₇R₈)ₙ(OCH₂CH₂)ₜ—, —K(CR₅R₆)ₘ(CR₇R₈)ₙ(OCH₂CH₂)ᵣ(Aa)ᵣ-, —K(CH₅R₆)ₘ—(CR₇═CR₈)(CR₉R₁₀)ₙ(Aa)ₜ(OCH₂CH₂)ᵣ—, —K(CR₅R₆)ₘ(NR₁₁CO)(Aa)ₜ(CR₉R₁₀)ₙ(OCH₂CH₂)ᵣ—, —K(CR₅R₆)ₘ(Aa)t(NR₁₁CO)(CR₉R₁₀)ₙ(OCH₂CH₂)ᵣ—, —K(CR₅R₆)ₘ(OCO)(Aa)ₜ(CR₉R₁₀)n-(OCH₂CH₂)ᵣ—, —K(CR₅R₆)ₘ(OCNR₇)(Aa)ₜ(CR₉R₁₀)ₙ(OCH₂CH₂)ᵣ—, —K(CR₅R₆)ₘ(CO)(Aa)ₜ-(CR₉R₁₀)ₙ(OCH₂CH₂)ᵣ—, —K(CR₅R₆)ₘ(NR₁₁CO)(Aa)ₜ(CR₉R₁₀)ₙ(OCH₂CH₂)ᵣ—, —K(CR₅R₆)ₘ—(OCO)(Aa)ₜ(CR₉R₁₀)ₙ(OCH₂CH₂)ᵣ—, —K(CR₅R₆)ₘ(OCNR₇)(Aa)r(CR₉R₁₀)ₙ(OCH₂CH₂)ᵣ—, —K(CR₅R₆)ₘ—(CR₅R₆)ₘ(CO)(Aa)ₜ(CR₉R₁₀)ₙ(OCH₂CH₂)ᵣ—, —K(CR₅R₆)ₘ-phenyl-CO(Aa)ₜ(CR₇R₈)ₙ—, —K—(CR₅R₆)ₘ-furyl-CO(Aa)ₜ-(CR₇R₈)ₙ—, —K(CR₅R₆)ₘ-oxazolyl-CO(Aa)ₜ(CR₇R₈)ₙ—, —K(CR₅R₆)ₘ-thiazolyl-CO(Aa)ₜ-(CR₇R₈)ₙ—, —K(CR₅R₆)ₜ-thienyl-CO(CR₇R₈)ₙ—, —K(CR₅R₆)ₜimidazolyl-CO—(CR₇R₈)ₙ—, —K(CR₅R₆)ₜmorpholino-CO(Aa)ₜ(CR₇R₈)ₙ—, —K(CR₅R₆)t piperazino-CO(Aa)ₜ-(CR₇R₈)ₙ—, —K(CR₅R₆)ₜ—N-methylpiperazinCO(Aa)ₜ(CR₇R₈)ₙ—, —K(CR₅R₆)ₘ(Aa)ₜphenyl, —K—(CR₇R₆)ₘ-(Aa)ₜfuryl-, —K(CR₅R₆)ₘ-oxazolyl(Aa)ₜ-, —K(CR₅R₆)ₘ-thiazolyl(Aa)ₜ-, —K(CR₅R₆)ₘ-thienyl-(Aa)ₜ-, —K(CR₅R₆)ₘ-imidazolyl(Aa)ₜ-, —K(CR₅R₆)ₘ-morpholino(Aa)ₜ-, —K(CR₅R₆)ₘ-piperazino-(Aa)ₜG, —K(CR₅R₆)ₘ-methylpiperazino(Aa)ₜ-; wherein m, Aa, m, and n are described above; t and r are 0-100 independently, R₃, R₄, R₅, R₆, R₇, and R₈ are independently H; halide; C₁˜C₈ alkyl; C₂˜C₈ aryl, alkenyl, alkynyl, ether, ester, amine or amide, which optionally substituted by one or more halide, CN, NR₁R₂, CF₃, OR₁, Aryl, heterocycle, S(O)R₁, SO₂R₁, —CO₂H, —SO₃H, —OR₁, —CO₂R₁, —CONR₁, —PO₂R₁R₂, —PO₃H or P(O)R₁R₂R₃; K is NR₁, —SS—, —S(═O)—, —C(═O)NH—, —C(═O)O—, —C═NH—O—, —C═N—NH—, —C(═O)NH—NH—, O, S, Se, B, Het (heterocyclic or heteroaromatic ring having C₁-C₈), or peptide containing 1-20 amino acids;
or L₁ and L₂ independently contain one following hydrophilic structures: 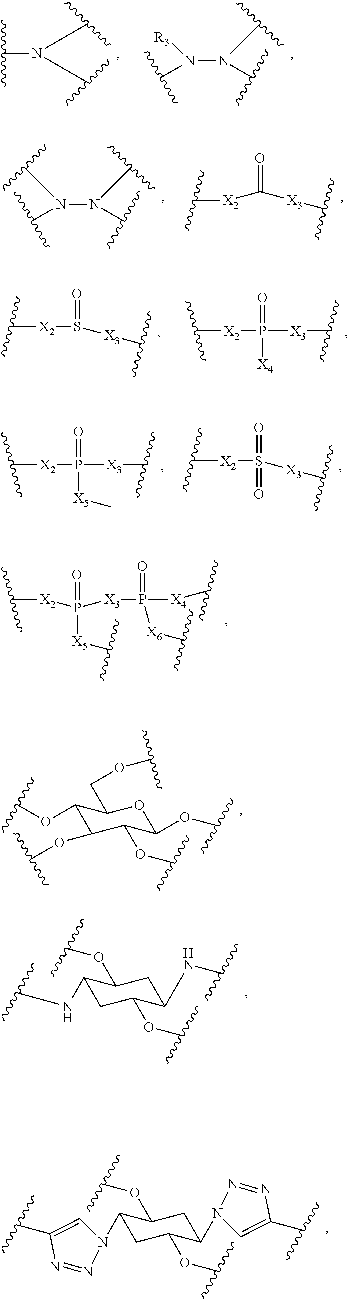 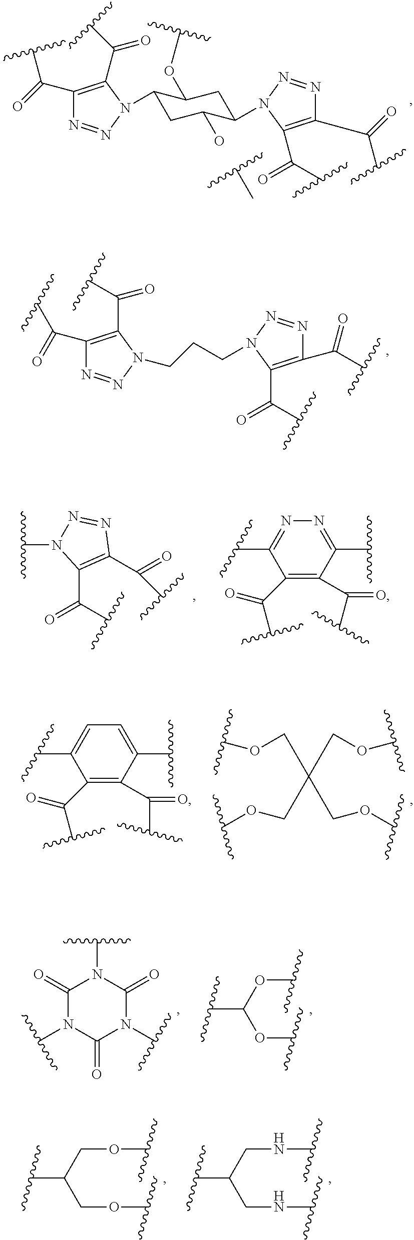 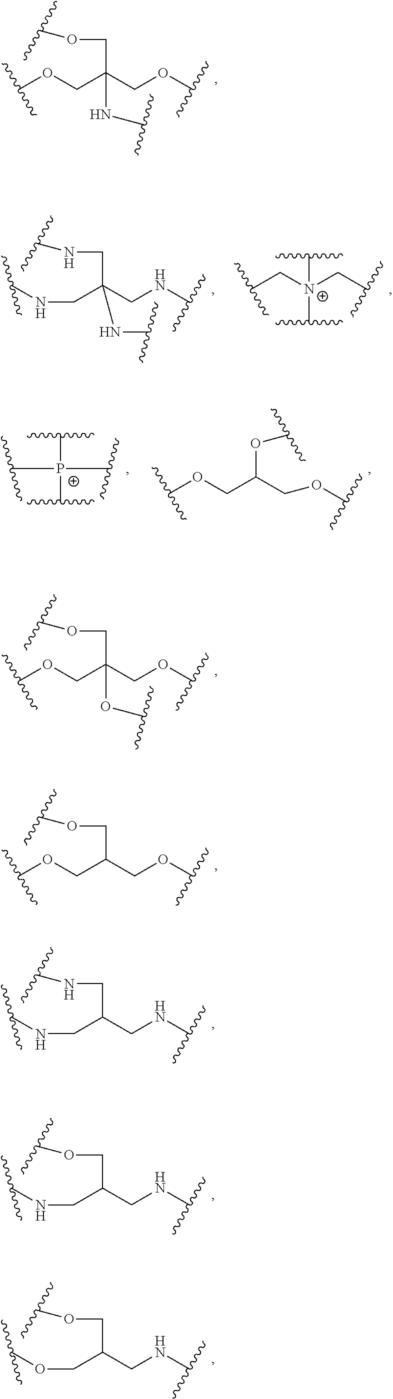
wherein [symbol] is a site of linkage; X₂, X₃, X₄, X₅, or X₆ are independently NH; NHNH; N(R₃); N(R₃)N(R_{3′}); O; S, C₁-C₆ alkyl; C₂-C₆ heteroalkyl, alkylcycloalkyl, or heterocycloalkyl; C₃-C₈ aryl, Ar-alkyl, heterocyclic, carbocyclic, cycloalkyl, heteroalkylcycloalkyl, alkylcarbonyl, or heteroaryl; or 1˜8 amino acids; wherein R₃ and R_{3′} are independently H; C₁-C₈ alkyl; C₂-C₈ hetero-alkyl, alkylcycloalkyl, or heterocycloalkyl; C₃-C₈ aryl, Ar-alkyl, heterocyclic, carbocyclic, cycloalkyl, heteroalkylcycloalkyl, alkylcarbonyl, or heteroaryl; or C₂-C₈ ester, ether, or amide; or polyethyleneoxy unit having formula (OCH₂CH₂)ₚ or (OCH₂CH(CH₃)ₚ, wherein p is an integer from 0 to about 5000, or a combination thereof;
or X, Y, L₁, L₂, Z₁ or Z₂ are independently composed of one or more following components: 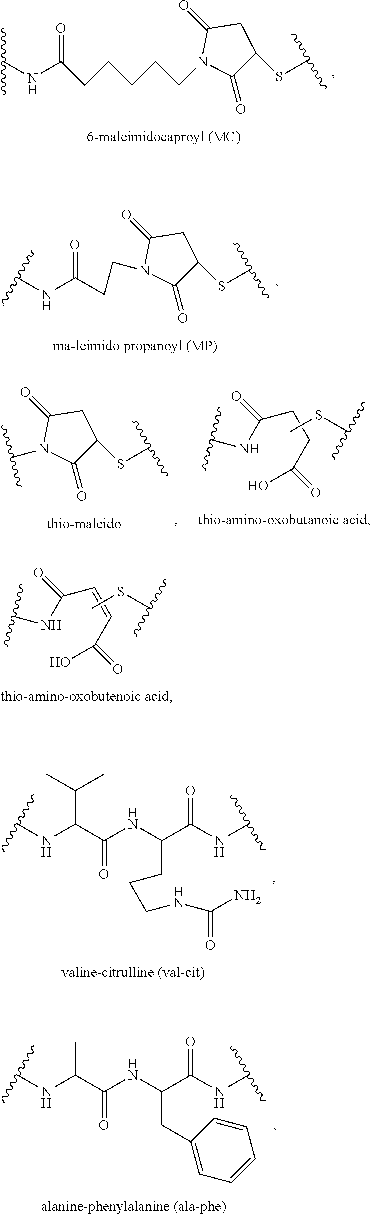 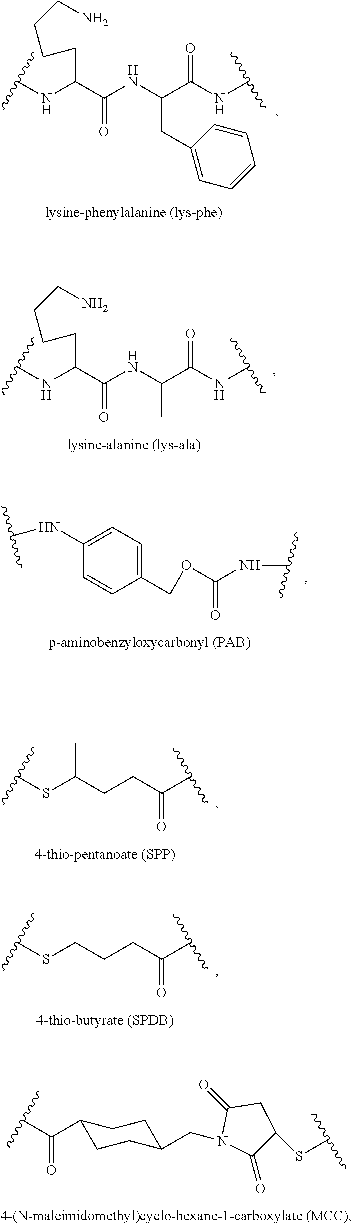 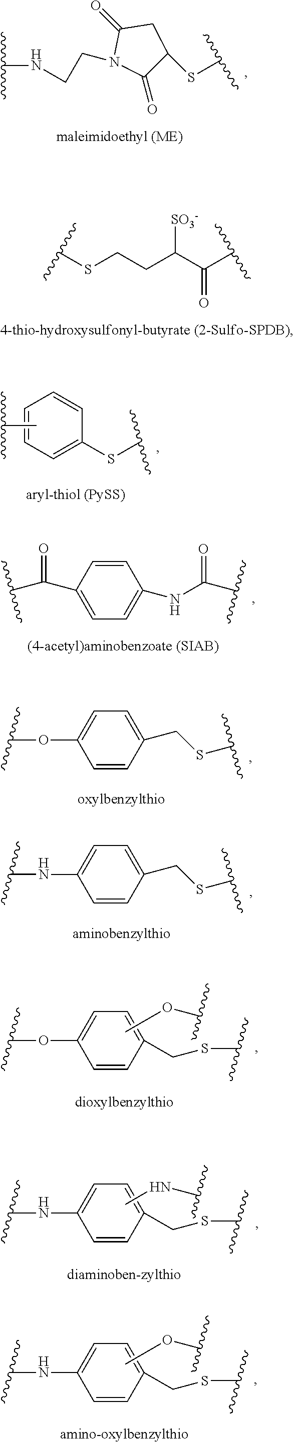 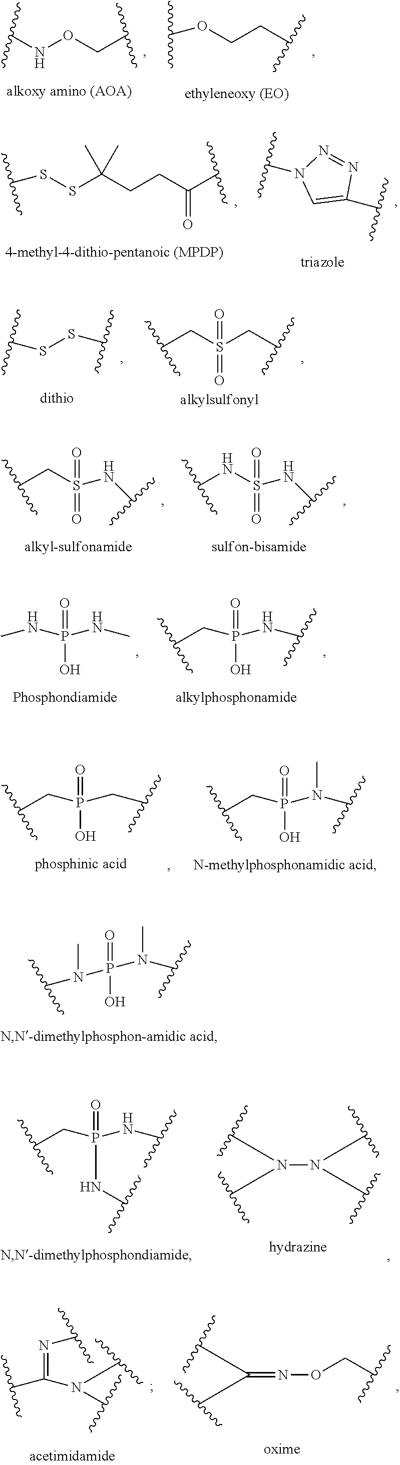 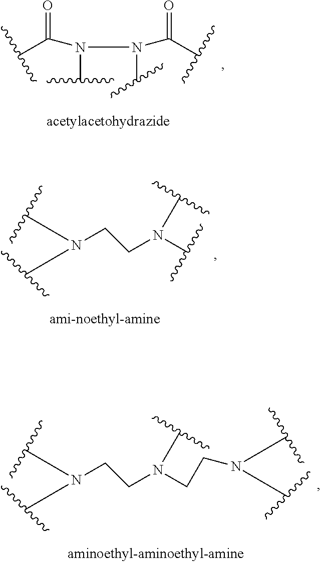 [SMILES: *C(=O)N(*)N(*)C(*)=O ; *N(*)CCN(*)* ; *N(*)CCN(*)CCN(*)*]
and L- or D-, natural or unnatural peptides containing 1-20 amino acids;
wherein [symbol] is a site that another bond is connected to:
or X, Y, L, Z₁, or Z₂, are independently absent, provided that L₁ and Z₁, or L₂ and Z₂ are not absent at the same time;
Lv₁ and Lv₂ represent the same or different leaving group that is capable of reacting with a thiol, amine, carboxylic acid, selenol, phenol or hydroxyl group on a cell-binding molecule; Lv₁ and Lv₂ are independently selected from OH; F; Cl; Br; I; nitrophenol; N-hydroxysuccinimide; phenol; dinitrophenol; pentafluorophenol; tetrafluorophenol; difluorophenol; mono-fluorophenol; pentachlorophenol; triflate; imidaole; dichlorophenol; tetrachlorophenol; 1-hydroxybenzotriazole; tosylate; mesylate; 2-ethyl-5-phenylisoxazolium-3′-sulfonate, anhydrides formed its self, or formed with the other anhydride: acetyl anhydride, or formyl anhydride; or an intermediate molecule generated with a condensation reagent for peptide coupling reactions, or for Mitsunobu reactions, which are selected from: N-(3-Dimethylaminopropyl)-N′-ethylcarbodiimide, Dicyclohexyl-carbodiimide, N,N′-Diisopropylcarbodiimide, N-Cyclohexyl-N′-(2-morpholino-ethyl)carbodiimide metho-p-toluenesulfonate, 1,1′-Carbonyldiimi-dazole, O-(Benzotriazol-1-yl)-N,N,N′,N′-tetramethyluronium tetrafluoroborate, N,N,N′,N′-Tetramethyl-O-(1H-benzotriazol-1-yl)-uronium hexafluorophosphate, (Benzotriazol-1-yloxy)tris(dimethylamino)-phosphonium hexafluorophosphate, (Benzotriazol-1-yloxy)tripyrrolidinophosphonium hexafluorophosphate, Diethyl cyanophosphonate, Chloro-N,N,N′,N′-tetramethylformamidiniumhexafluorophosphate, 1-[Bis(dimethylamino)methylene]-1H-1,2,3-triazolo[4,5-b]pyridinium 3-oxid hexafluorophos-phate, 1-[(Dimethylamino)(morpho-lino)methylene]-1H-[1,2,3]triazolo[4,5-b]pyridine-1-ium 3-oxide hexafluoro-phosphate, 2-Chloro-1,3-dimethyl-imidazolidinium hexafluorophosphate, Chlorotripyrrolidinophosphonium hexafluorophosphate, Fluoro-N,N,N′,N′-bis(tetramethylene)-formamidinium hexafluorophosphate, N,N,N′,N′-Tetramethyl-S-(1-oxido-2-pyridyl)thiuronium hexafluorophosphate, O-(2-Oxo-1 (2H)pyridyl)-N,N,N′,N′-tetramethyl-uronium tetrafluoroborate, S-(1-Oxido-2-pyridyl)-N,N,N′,N′-tetramethylthiuronium tetrafluoroborate, O-[(Ethoxycarbonyl)-cyanomethylenamino]-N,N,N′,N′-tetramethyluronium hexafluorophosphate, (1-Cyano-2-ethoxy-2-oxoethylidenaminooxy) dimethylamino-morpholino-carbenium hexafluorophosphate, O-(Benzotriazol-1-yl)-N,N,N′,N′-bis(tetramethylene)uronium hexafluorophosphate, N-Benzyl-N′-cyclohexyl-carbodiimide (with, or without polymer-bound), Dipyrrolidino(N-succinimidyl-oxy)carbenium hexafluoro-phosphate, Chlorodipyrrolidinocarbenium hexafluorophosphate, 2-Chloro-1,3-dimethylimidazolidinium tetrafluoroborate, (Benzotriazol-1-yloxy)dipiperi-dinocarbenium hexafluorophosphate, O-(6-Chlorobenzotriazol-1-yl)-N,N,N′,N′-tetramethyluronium tetrafluoroborate, Bromotris(dimethylamino)-phosphonium hexafluorophosphate, Propylphosphonic anhydride, 2-Morpholinoethyl isocyanide, N,N,N′,N′-Tetramethyl-O—(N-succinimidyl)uronium hexafluorophosphate, 2-Bromo-1-ethyl-pyridinium tetrafluoroborate, O-[(Ethoxycarbonyl)cyano-methylenamino]-N,N,N′,N′-tetra-methyluronium tetrafluoroborate, 4-(4,6-Dimethoxy-1,3,5-triazin-2-yl)-4-methylmorpholiniumchloride, N,N,N′,N′-Tetramethyl-O—(N-succinimidyl)uronium tetrafluoroborate, O-(3,4-Dihydro-4-oxo-1,2,3-benzotriazin-3-yl)-N,N,N′,N′-tetramethyluronium tetrafluoro-borate, 1,1′-(Azodicarbonyl)-dipiperidine, Di-(4-chlorobenzyl)azodicarboxylate, Di-tert-butyl azodicarboxylate, Diisopropyl azodicarboxylate, Diethyl azodicarboxylate, or Lv₁ and Lv₂ are an anhydride, formed by acid themselves or formed with other C₁˜C₈ acid anhydrides;
or Lv₁ and Lv₂ are independently a halide, methanesulfonyl, toluenesulfonyl, trifluoromethyl-sulfonyl, trifluoromethylsulfonate, nitrophenoxyl, N-succinimidyloxyl, phenoxyl; dinitrophenoxyl; pentafluorophenoxyl, tetrafluorophenoxyl, trifluorophenoxyl, difluorophenoxyl, monofluorophenoxyl, pentachlorophenoxyl, 1H-imidazole-1-yl, chlorophenoxyl, dichlorophenoxyl, trichlorophenoxyl, tetrachlorophenoxyl, N-(benzotriazol-yl)oxyl, 2-ethyl-5-phenylisoxazolium-3′-sulfonyl, phenyloxadiazole-sulfonyl (-sulfone-ODA), 2-ethyl-5-phenylisoxazolium-yl, phenyloxadiazol-yl, oxadiazol-yl, unsaturated carbon (a double or a triple bond between carbon-carbon, carbon-nitrogen, carbon-sulfur, carbon-phosphorus, sulfur-nitrogen, phosphorus-nitrogen, oxygen-nitrogen, or carbon-oxygen), or one of following structure: 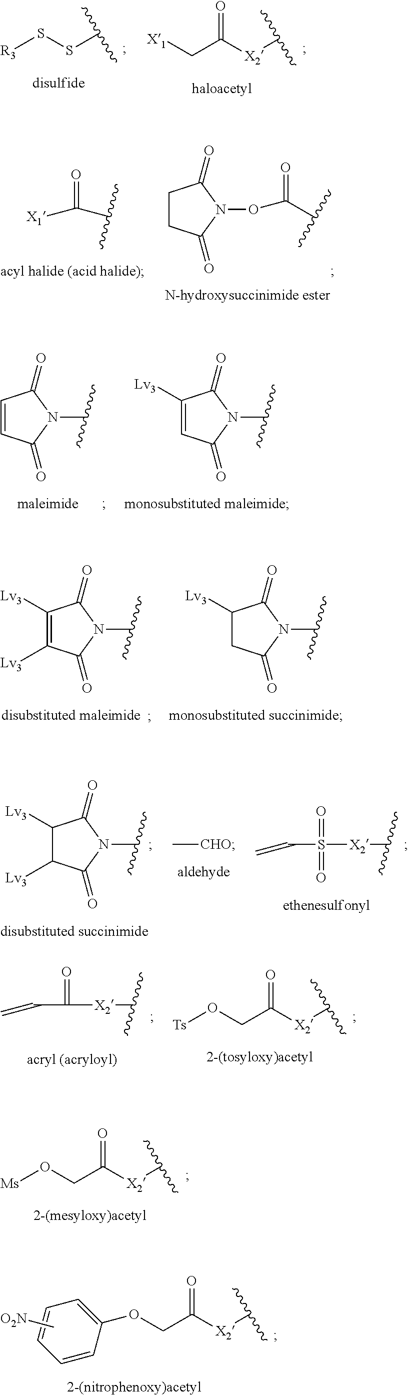 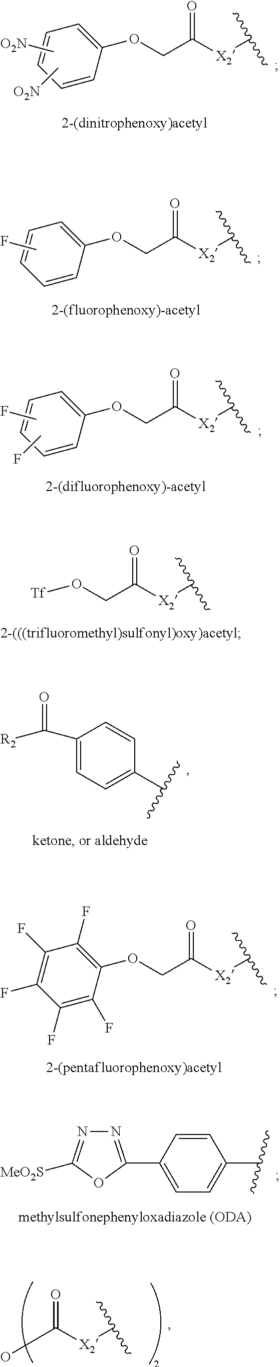 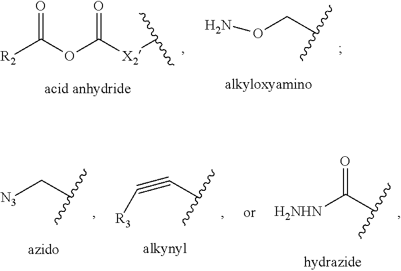
wherein X₁′ is F, Cl, Br, I or Lv₃; X₂′ is O, NH, N(R₁), or CH₂; R₃ is independently H, aromatic, heteroaromatic, or aromatic group wherein one or several H atoms are replaced independently by —R₁, -halogen, —OR₁, —SR₁, —NR₁R₂, —NO₂, —S(O)R₁, —S(O)₂R₁, or —COOR₁; Lv₃ is a leaving group selected from F, Cl, Br, I, nitrophenol; N-hydroxysuccinimide (NHS); phenol; dinitrophenol; pentafluorophenol; tetrafluorophenol; difluorophenol; monofluorophenol; pentachlorophenol; triflate; imidazole; dichlorophenol; tetrachlorophenol; 1-hydroxybenzotriazole; tosylate; mesylate; and 2-ethyl-5-phenylisoxazolium-3′-sulfonate; R₁ and R₂ are independently H, C₁-C₈ alkyl, C₂-C₈ alkenyl, heteroalkyl, alkylcycloalkyl, or heterocycloalkyl; C₃-C₈ aryl, Ar-alkyl, heterocyclic, carbocyclic, cycloalkyl, heteroalkylcycloalkyl, alkylcarbonyl, or heteroaryl, or C₂-C₈ ester, ether, or amide; or peptide containing 1-8 amino acids; or polyethyleneoxy unit having formula (OCH₂CH₂)ₚ or (OCH₂CH(CH₃))ₚ, wherein p is an integer from 0 to about 5000, or a combination thereof;
wherein the compound of Formula (II) excludes following structure: 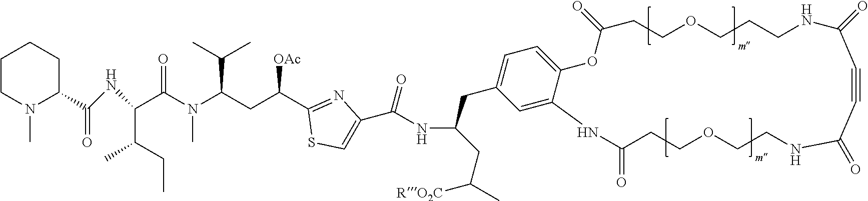
wherein m″=1-3; R′″═H, CH₃ or C₂H₅.

3. A bis-linker having conjugated to a cell-binding molecule of Formula (III): 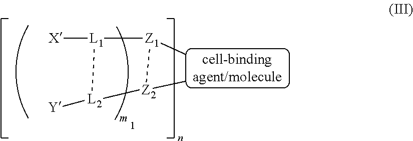
wherein:
“ [symbol] ” represents a sing bond; “ [symbol] ” is a single bond, a double bond, or absent; n and m₁ are 1 to 20 independently;
a cell-binding molecule that links to Z₁ and Z₂ is a molecule that binds to, complexes with, or reacts with a moiety of a target cell, the cell-binding molecule being an immunotherapeutic protein, an antibody, a single chain antibody; an antibody fragment that binds to the target cell; a monoclonal antibody; a single chain monoclonal antibody; or a monoclonal antibody fragment that binds to the target cell; a chimeric antibody; a chimeric antibody fragment that binds to the target cell; a domain antibody; a domain antibody fragment that binds to the target cell; adnectins that mimic antibodies; DARPins; a lymphokine; a hormone; a vitamin; a growth factor; a colony stimulating factor; or a nutrient-transport molecule; a transferrin; a binding peptide having at least four aminoacids; or an antibody, a protein, a small cell-binding molecule or a binding-ligand attached on albumin, polymers, dendrimers, liposomes, nanoparticles, vesicles, or on (viral) capsids;
Z₁ and Z₂ are, the same or different, and independently a function group that is linked to the cell-binding molecule, to form a disulfide, ether, ester, thioether, thioester, peptide hydrazone, carbamate, carbonate amine (secondary, tertiary, or quarter), imine, cycloheteroalkyane, heteroaromatic, alkyloxime or amide body; Z₁ and Z₂ independently are: C(O)CH, C(O)C, C(O)CH₂, ArCH₂, C(O), NH; NHNH; N(R₁); N(R₁)N(R₂); O; S; S—S, O—NH, O—N(R₁), CH₂—NH, CH₂—N(R₁), CH═NH, CH—N(R₁), S(O), S(O₂), P(O)(OH), S(O)NH, S(O₂)NH, P(O)(OH)NH, NHS(O)NH, NHS(O₂)NH, NHP(O)(OH)NH, N(R₁)S(O)N(R₂), N(R₁)S(O₂)N(R₂), N(R₁)P(O)(OH)N(R₂), OS(O)NH, OS(O₂)NH, OP(O)(OH)NH, C(O), C(NH), C(NR₁), C(O)NH, C(NH)NH, C(NR₁)NH, OC(O)NH, OC(NH)NH; OC(NR₁)NH, NHC(O)NH; NHC(NH)NH; NHC(CR₁)NH, C(O)NH, C(NH)NH, C(NR₁)NH, OC(O)N(R₁), OC(NH)N(R₁), OC(NR₁)N(R₁), NHC(O)N(R₁), NHC(NH)N(R₁), NHC(NR₁)N(R₁), N(R₁)C(O)N(R₁), N(R₁)C(NH)N(R₁), N(R₁)C(NR₁)N(R₁); C₁-C₈ alkyl, C₂-C₈ heteroalkyl, alkylcycloalkyl, or heterocycloalkyl; or C₃-C₈ aryl, Ar-alkyl, heterocyclic, carbocyclic, cycloalkyl heteroalkylcycloalkyl, alkylcarbonyl, or heteroaryl;
L₁ and L₂ are, the same or different, independently selected from O; NH; S; NHNH; N(R₃); N(R₃)N(R_{3′}); C₁-C₈ alkyl, amide, amines, imines, hydrazines, or hydrazones; C₂-C₈ heteroalkyl, alkylcycloalkyl, ethers, esters, hydrazones, ureas, semicarbazides, carbazides, alkoxyamines, alkoxylamines, urethanes, amino acids, peptides, acyloxylamines, hydroxamic acids, or heterocycloalkyl; C₃-C₈ aryl, Ar-alkyl, heterocyclic, carbocyclic, cycloalkyl, heteroalkylcycloalkyl, alkylcarbonyl, or heteroaryl, 1˜8 amino acids; and polyethyleneoxy unit of formula (OCH₂CH₂)ₚOR₃, or(OCH₂—CH(CH₃)ₚOR₃, or NH(CH₂)CH₂O)ₚR₃, or NH(CH₂CH(CH₃)O)ₚR₃, or N[(CH₂CH₂O)ₚR₃]—[(CH₂CH₂O)_{p′}R_{3′}], or (OCH₂CH₂)ₚCOOR₃, or CH₂CH₂(OCH₂CH₂)ₚCOOR₃, wherein p and p′ are independently an integer selected from 0 to about 5000, or a combination thereof; R₃ and R_{3′} are independently H; C₁-C₈ alkyl, C₂-C₈ heteroalkyl, alkylcycloalkyl, or heterocycloalkyl; C₃-C₈ aryl, Ar-alkyl, heterocyclic, carbocyclic, cycloalkyl, heteroalkylcycloalkyl, alkylcarbonyl, or heteroaryl; or C₂-C₈ ester, ether, or amide; or 1˜8 amino acids; or polyethyleneoxy having formula (OCH₂CH₂)ₚ or (OCH₂—CH(CH₃))ₚ, wherein p is an integer from 0 to about 5000, or a combination thereof;
or L₁ and L₂ independently have one or more linker components of 6-maleimidocaproyl (“MC”), maleimidopropanoyl (“MP”), valine-citrulline (“val-cit” or “vc”), alanine-phenylalanine (“ala-phe” or “af”), p-aminobenzyloxycarbonyl (“PAB”), 4-thiopentanoate (“SPP”), 4-(N-maleimidomethyl)cyclohexane-1 carboxylate (“MCC”), (4-acetyl)amino-benzoate (“SIAB”), 4-thio-butyrate (SPDB), 4-thio-2-hydroxysulfonyl-butyrate (2-Sulfo-SPDB), or natural or unnatural peptide having 1˜8 natural or unnatural amino acid units;
or L₁ and L₂ independently contain a self-immolative component, peptidic unit, a hydrazone bond, a disulfide, an ester, an oxime, an amide, or a thioether bond; the self-immolative unit being an aromatic compound that is electronically similar to para-aminobenzyl-carbamoyl (PAB) groups, 2-aminoimidazol-5-methanol derivatives, heterocyclic PAB analogs, beta-glucuronide, and ortho or para-aminobenzylacetals; or one of following structures: 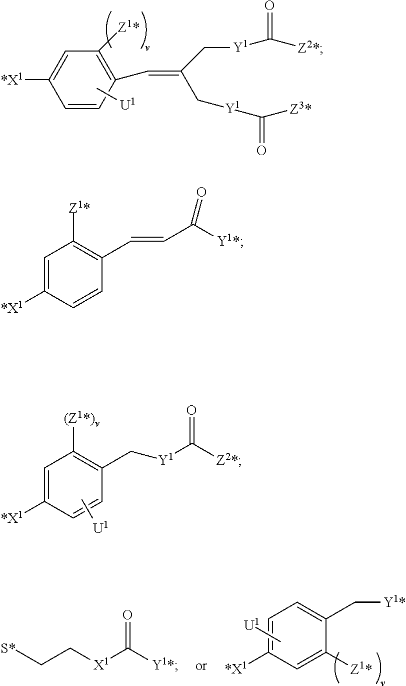
wherein the (*) atom is a point of attachment of additional spacer or releasable linker units or the cell-binding molecule; X¹, Y¹, Z² and Z³ are independently, NH, O, or S; Z¹ is independently H, NHR₁, OR₁, SR₁, COX₁R₁, wherein X₁ and R₁ are defined above; v is 0 or 1; U¹ is independently H, OH, C₁˜C₆ alkyl, (OCH₂CH₂)ₙ, F, Br, I, OR₅, SR₅, NR₅R₅′, N═NR₅, N═R₅, NR₅R₅′, NO₂, SOR₅R₅′, SO₂R₅, SO₃R₅, OSO₃R₅, PR₅R₅′, POR₅R₅′, PO₂R₅R₅′, OPO(OR₅)(OR₅′), or OCH₂PO(OR₅(OR₅′), wherein R₅ and R₅′ are independently H, C₁˜C₈ alkyl; C₂˜C₈ alkenyl, alkynyl, heteroalkyl, or amino acid; C₃˜C₈ aryl, heterocyclic, carbocyclic, cycloalkyl, heterocycloalkyl, heteroalkyl, alkylcarbonyl, or glycoside, or pharmaceutical cation salts;
or L₁ and L₂ independently have a non-self-immolative linker component containing one of following structures: 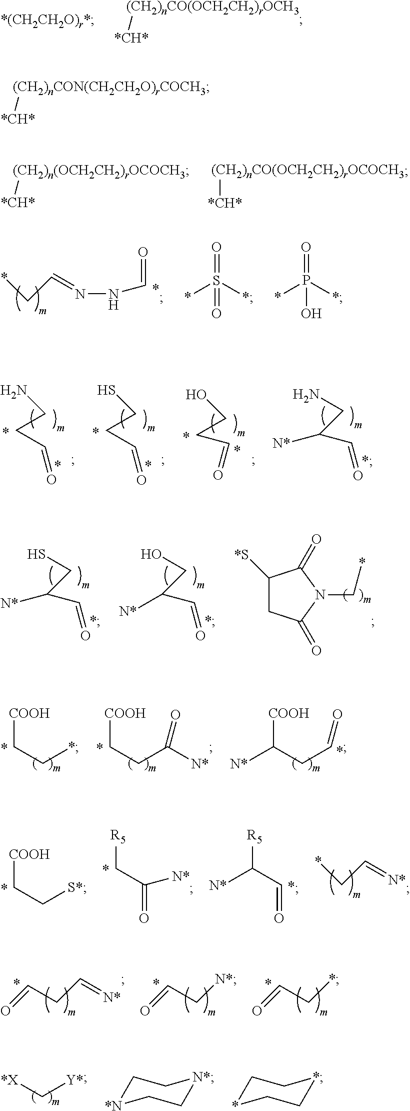 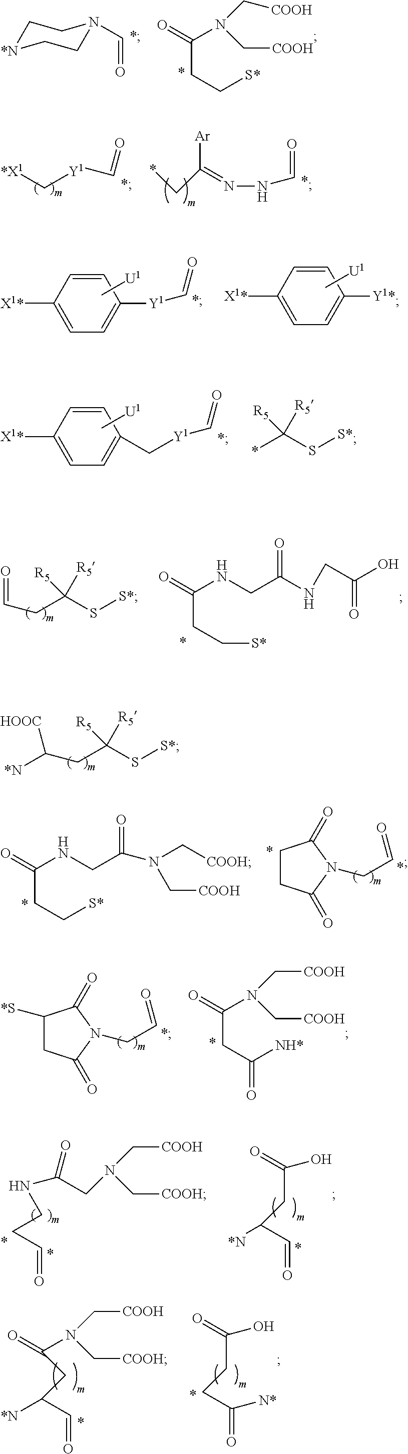 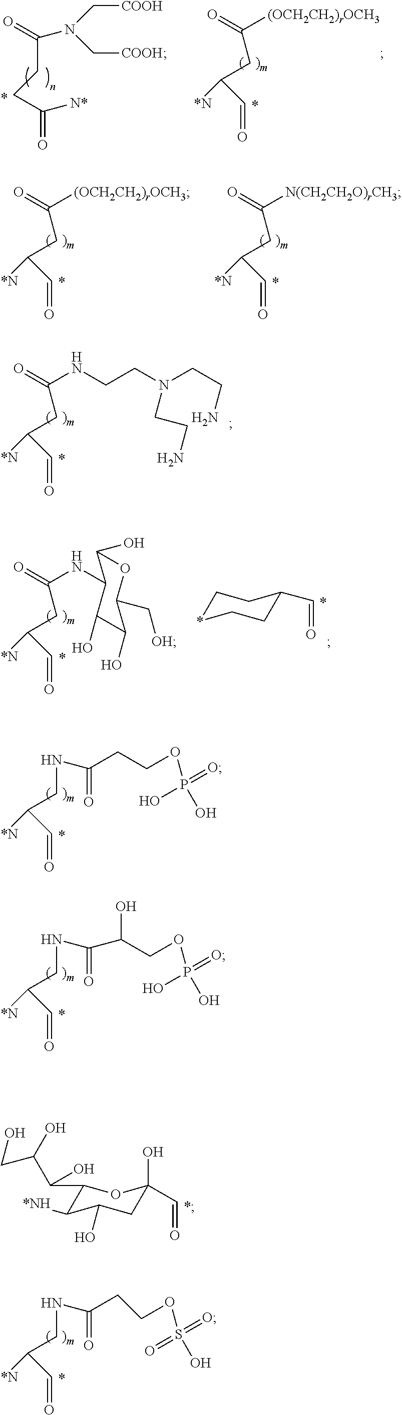 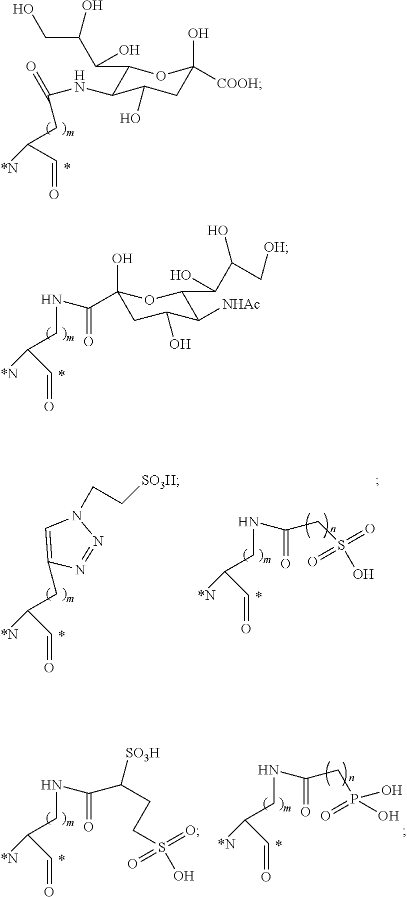
wherein the (*) atom is a point of attachment of additional spacer or releasable linkers, the cell-binding molecule; X¹, Y¹, U¹, R₅, R₅′ are defined as above; r is 0˜100; m and n are 0˜6 independently;
or L₁ and L₂ independently are a releasable linker containing at least one bond that is capable of being broken under physiological conditions: a pH-labile, acid-labile, base-labile, oxidatively labile, metabolically labile, biochemically labile or enzyme-labile bond, having one of following structures: —(CR₅R₆)ₘ(Aa)r(CR₇R₈)ₙ(OCH₂CH₂)ₜ—, —(CR₅R₆)ₘ(CR₇R₈)ₙ(Aa)ₜ(OCH₂CH₂)ₜ—, -(Aa)ᵣ-(CR₅R₆)ₘ(CR₇R₈)ₙ(OCH₂CH₂)ₜ—, —(CR₅R₆)ₘ(CR₇R₈)ₙ(OCH₂CH₂)ᵣ(Aa)ₜ-, —(CR₅R₆)ₘ—(CR₇═CR₈)(CR₉R₁₀)ₙ(Aa)ₜ(OCH₂CH₂)ᵣ—, —(CR₅R₆)ₘ(NR₁₁CO)(Aa)ₜ(CR₉R₁₀)ₙ(OCH₂CH₂)ᵣ—, —(CR₅R₆)ₘ(Aa)(NR₁₁CO)(CR₉R₁₀)ₘ(OCH₂CH₂)ᵣ—, —(CR₅R₆)ₘ(OCO)(Aa)ₜ(CR₉R₁₀)ₙ(OCH₂CH₂)ᵣ—, —(CR₅R₆)ₘ(OCNR₇)(Aa)ₜ(CR₉R₁₀)ₙ(OCH₂CH₂)ᵣ—, —(CR₅R₆)ₘ(CO)(Aa)ₜ-(CR₉R₁₀)ₙ(OCH₂CH₂)ₜ—, —(CR₅R₆)ₘ(NR₁₁CO)(Aa)ₜ(CR₉R₁₀)ₙ(OCH₂CH₂)ᵣ—, —(CR₅R₆)ₘ(OCO)(Aa)ₜ(CR₉R₁₀)ₙ—(OCH₂CH₂)ᵣ—, (CR₅R₆)ₘ(OCNR₇)(Aa)ᵣ(CR₉R₁₀)ₙ(OCH₂CH₂)ₜ—, —(CR₅R₆)ₘ(CO)(Aa)ₜ(CR₉R₁₀)ₙ(OCH₂CH₂)ₜ—, —(CR₅R₆)ₘ-phenyl-CO(Aa)ₜ(CR₇R₈)ₙ—, —(CR₅R₆)ₘ-furyl-CO(Aa)ₜ(CR₇R₈)ₙ—, —(CR₅R₆)ₘ-oxazolyl-CO(Aa)ₜ(CR₇R₈)ₙ—, —(CR₅R₆)ₘ-thiazolyl-CO(Aa)ₜ(CCR₇R₈)ₙ—, —(CR₅R₆)ₜ-thienyl-CO(CR₇R₈)ₙ—, —(CR₅R₆)ₜ-imidazolyl-CO—(CR₇R₈)ₙ—, —(CR₅R₆)ₜ-morpholino-CO(Aa)ₜ(CR₇R₈)ₙ—, —(CR₇R₈)ₙ—, —(CR₅R₆)t piperazino-CO(Aa)ₜ(CR₇R₈)ₙ—, —(CR₅R₆)ₜ—N-methylpiperazin-CO(Aa)ₜ(CR₇R₈)ₙ—, —(CR₅R₆)ₘ-(Aa)ₜphenyl-, —(CR₅R₆)ₘ-(Aa)ₜfuryl-, —(CR₅R₆)ₘ-oxazolyl(Aa)ₜ-, —(CR₅R₆)ₘ-thiazolyl(Aa)ᵣ-, —(CR₅R₆)ₘ-thienyl-(Aa)ₜ-, —(CR₅R₆)ₘ-imidazolyl(Aa)ₜ-, —(CR₅R₆)ₘ-morpholino-(Aa)ₜ-, —(CR₅R₆)ₘ-piperazino-(Aa)ₜ-, —(CR₅R₆)ₘ—N-methylpiperazino-(Aa)ₜ-, —K(CR₅R₆)ₘ(Aa)ᵣ(CR₇R₈)ₙ(OCH₂CH₂)ₜ—, —K(CR₅R₆)ₘ(CR₇R₈)ₙ(Aa)r(OCH₂CH₂)ₜ—, —K(Aa)ₜ-(CR₅R₆)ₘ(CR₇R₈)ₙ(OCH₂CH₂)ₜ—, —K(CR₅R₆)ₘ(CR₇R₈)ₙ(OCH₂CH₂)ᵣ(Aa)ₜ-, —K(CR₅R₆)ₘ—(CR₇═CR₈)(CR₉R₁₀)ₙ(Aa)r(OCH₂CH₂)ᵣ—, —K(CR₅R₆)ₘ(NR₁₁CO)(Aa)ₜ(CR₉R₁₀)ₙ(OCH₂CH₂)ₜ—, —K(CR₅R₆)ₘ((Aa)ₜ(NR₁₁CO)(CR₉R₁₀)ₙ(OCH₂CH₂)ₜ—, —K(CR₅R₆)m)(OCO)(Aa)ₜ(CR₉R₁₀)ₙ—(OCH₂CH₂)ᵣ—, —K(CR₅R₆)ₘ(OCNR₇)(Aa)ₜ(CR₉R₁₀)ₙ(OCH₂CH₂)ₜ—, —K(CR₅R₆)ₘ(CO)(Aa)ₜ-(CR₉R₁₀)ₙ(OCH₂CH₂)ᵣ—, —K(CR₅R₆)ₘ(NR₁₁CO)(Aa)ₜ(CR₉R₁₀)ₘ(OCH₂CH₂)ᵣ—, —K(CR₅R₆)ₘ—(OCO)(Aa)ₜ(CR₉R₁₀)ₙ(OCH₂CH₂)ₜ—, —K(CR₅R₆)ₘ(OCNR₇)(Aa)ₜ(CR₉R₁₀)ₙ(OCH₂CH₂)ₜ—, —K—(CR₅R₆)ₘ(CO)(Aa)ₜ(CR₉R₁₀)ₙ(OCH₂CH₂)ᵣ—, —K(CR₅R₆)ₘ-phenyl-CO(Aa)ₜ(CR₇R₈)ₙ—, —K—(CR₅R₆)ₘ-furyl-CO(Aa)ₜ-(CR₇R₈)ₙ—, —K(CR₅R₆)ₘ-oxazolyl-CO(Aa)ₜ(CR₇R₈)ₙ—, —K(CR₅R₆)ₘ-thiazolyl-CO(Aa)ₜ-(CR₇R₈)ₙ—, —K(CR₅R₆)ₜ-thienyl-CO(CR₇R₈)ₙ—, —K(CR₅R₆)t imidazolyl-CO—(CR₇R₈)ₙ—, —K(CR₅R₆)ₜmorpholino-CO(Aa)ₜ(CR₇R₈)ₙ—, —K(CR₅R₆)t piperazino-CO(Aa)ₜ-(CR₇R₈)ₙ—, —K(CR₅R₆)ₜ—N-methylpiperazinCO(Aa)ₜ(CR₇R₈)ₙ—, —K(CR₅R₆)ₘ(Aa)ₜphenyl, —K—(CR₅R₆)ₘ, (Aa)ₜfuryl-, —K(CR₅R₆)ₘ-oxazolyl(Aa)ₜ-, —K(CR₅R₆)ₘ-thiazolyl(Aa)ₜ-, —K(CR₅R₆)ₘ-thienyl-(Aa)ₜ-, —K(CR₅R₆)ₘ-imidazolyl(Aa)ₜ-, —K(CR₅R₆)ₘ-morpholino(Aa)ₜ-, —K(CR₅R₆)ₘ-piperazino-(Aa)ₜG, —K(CR₅R₆)ₘN-methylpiperazino(Aa)ₜ-; wherein Aa, m, and n are described above; t and r are 0-100 independently; R₃, R₄, R₅, R₆, R₇, and R₈ are independently H; halide; C₁˜C₈ aryl, alkenyl, alkynyl, ether, ester, amine or amide, which optionally substituted by one or more halide, CN, NR₁R₂, CF₃, OR₁, Aryl, heterocycle, S(O)R₁, SO₂R₁, —CO₂H, —SO₃H, —OR₁, —CO₂R₁, —CONR₁, —PO₂R₁R₂, —PO₃H or P(O)R₁R₂R₃; K is NR₁, —SS—, —C(═O)—, —C(═O)NH—, —C(═O)O—, —C═NH—O—, —C═N—NH, —C(═O)NH—NH—, O, S, Se, B, Het (heterocyclic or heteroaromatic ring having C3-C8), or peptide containing 1-20 amino acids;
or L₁ and L₂ independently contain one of following hydrophilic structures: 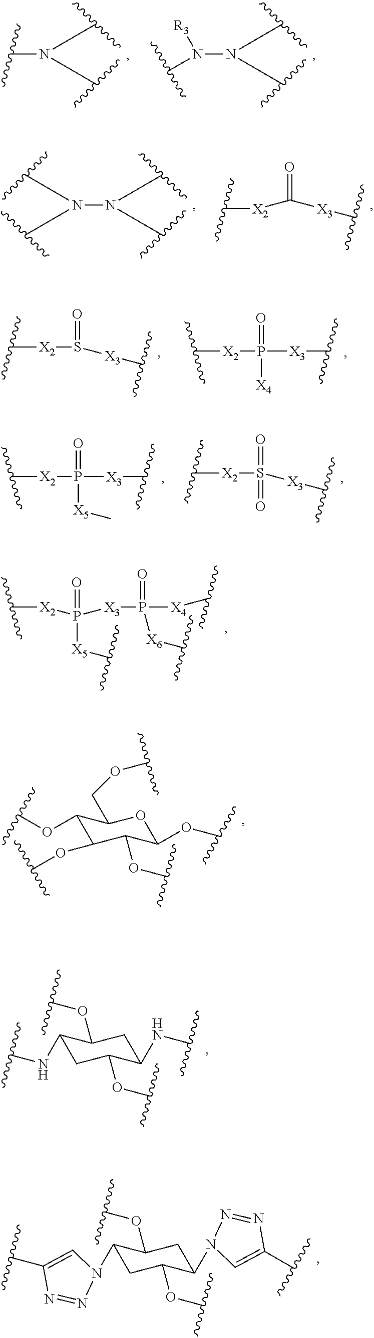 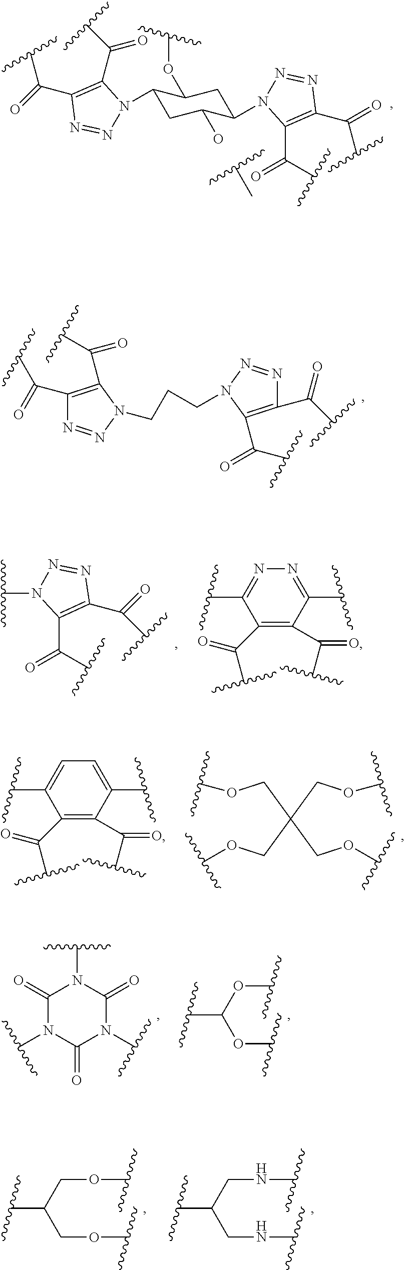 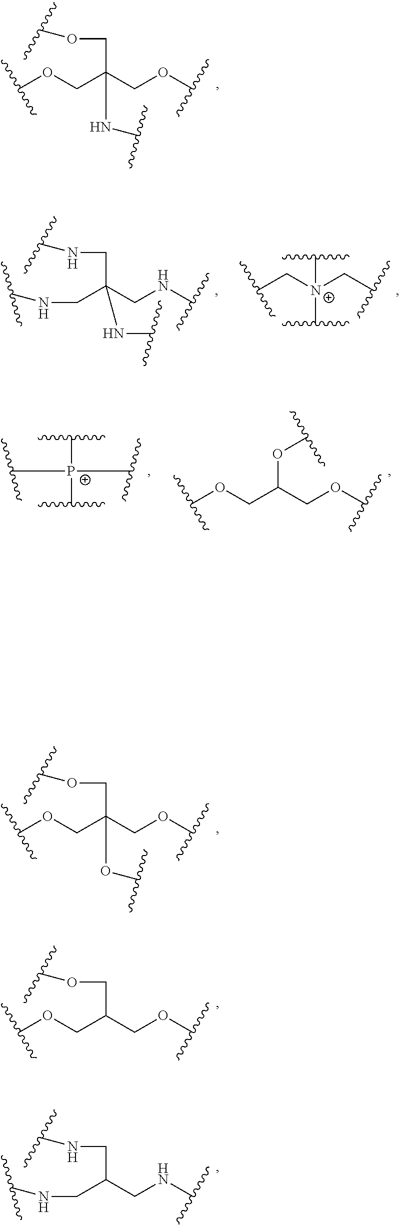 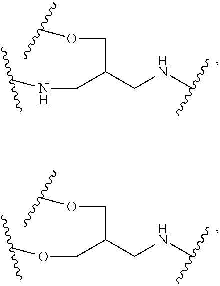
wherein [symbol] is a site of linkage; X₂, X₃, X₄, X₅, or X₆ are independently NH; NHNH; N(R₃); N(R₃)N(R_{3′}); O; S; C₁-C₆ alkyl; C₂-C₆ heteroalkyl, alkylcycloalkyl, or heterocycloalkyl; C₃-C₈ aryl, Ar-alkyl, heterocyclic, carbocyclic, cycloalkyl, heteroalkylcycloalkyl, alkylcarbonyl, or heteroaryl; or 1˜8 amino acids; wherein R₃ and R_{3′} are independently H; C₁-C₈ alkyl; C₂-C₈ hetero-alkyl, alkylcycloalkyl, or heterocycloalkyl; C₃-C₈ aryl, Ar-alkyl, heterocyclic, carbocyclic, cycloalkyl, heteroalkylcycloalkyl, alkylcarbonyl, or heteroaryl; or C₃-C₈ ester, ether or amide; or polyethyleneoxy unit having formula (OCH₂CH₂)ₚ or (OCH₂CH(CH₃))ₚ, wherein p is an integer from 0 to about 5000, or a combination thereof;
or L₁, L₂, Z₁ or Z₂ are independently composed of one or more following components: 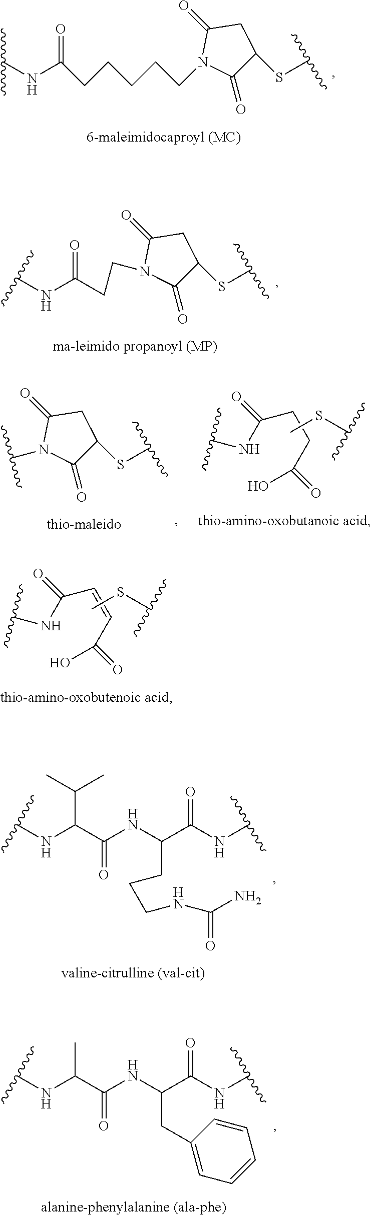 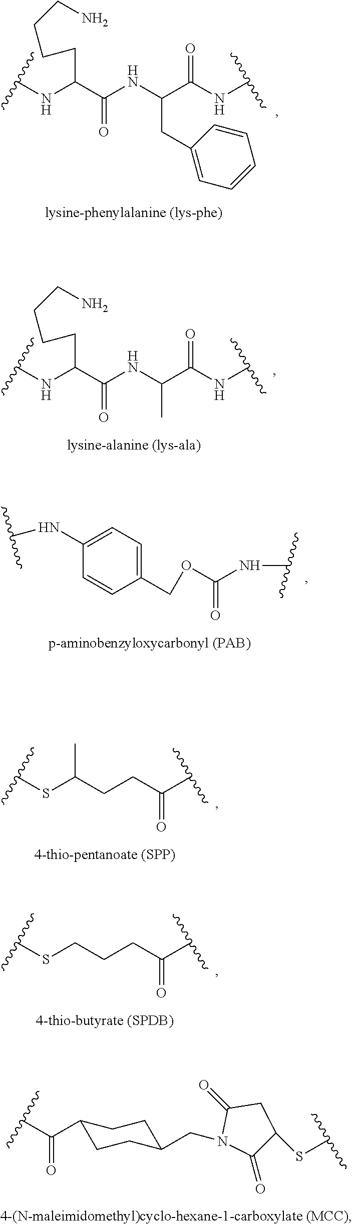 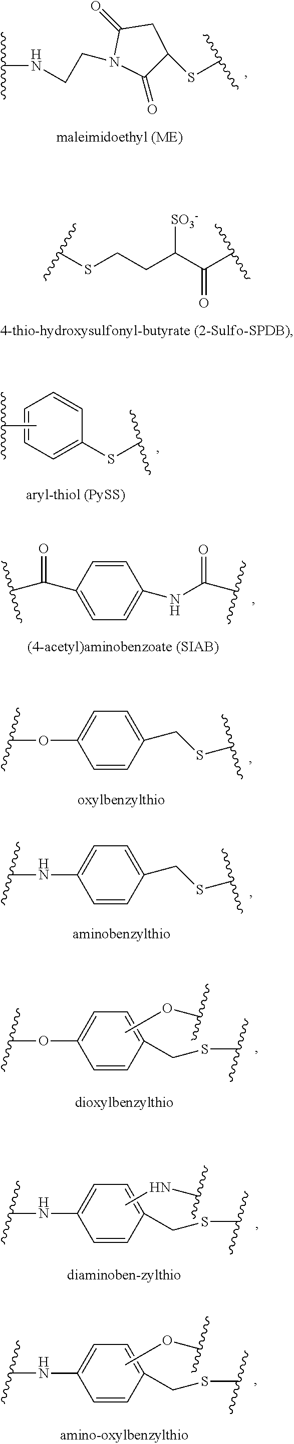 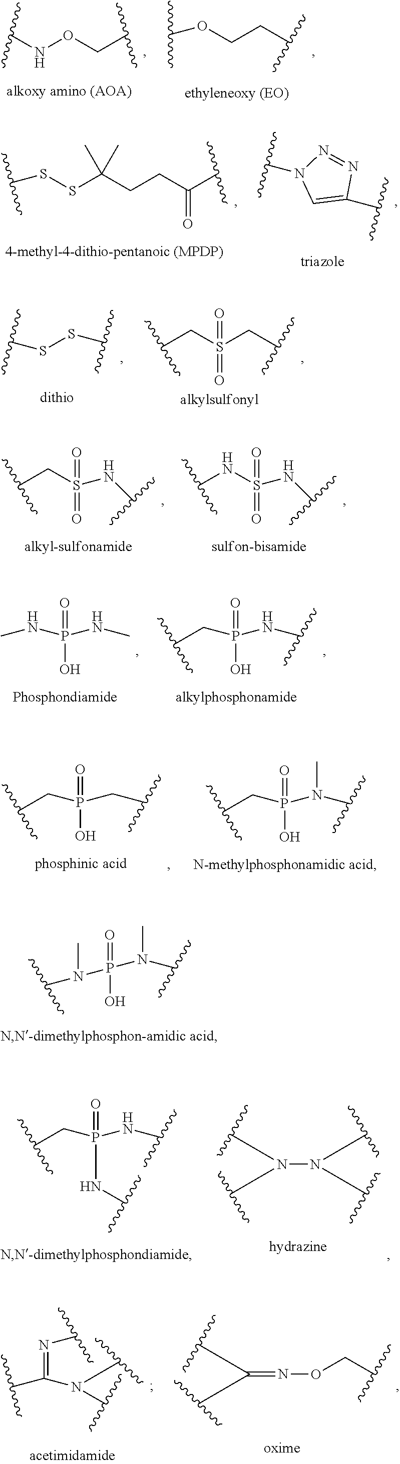 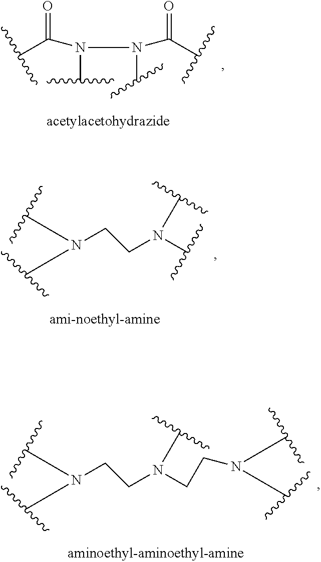 [SMILES: *C(=O)N(*)N(*)C(*)=O ; *N(*)CCN(*)* ; *N(*)CCN(*)CCN(*)*]
and L- or D-, natural or unnatural peptides containing 1-20 amino acids;
wherein [symbol] is a site that another bond is connected to:
or L₁, L₂, Z₁, Z₂, are independently absent, provided that L₁ and Z₁, or L₂ and Z₂ are not absent at the same time;
X′ and Y′ are a function group that is capable of independently reacting with a residue group of a cytotoxic drug simultaneously or sequentially;
X′ and Y′ are independently a disulfide substituent, maleimido, haloacetyl, alkoxyamine, azido, ketone, aldehyde, hydrazine, amino, hydroxyl, carboxylate, imidazole, thiol, or alkyne; or a N-hydroxysuccinimide ester, p-nitrophenyl ester, dinitrophenyl ester, pentafluorophenyl ester, pentachlorophenyl ester; tetrafluorophenyl ester; difluorophenyl ester; monofluorophenyl ester; or pentachlorophenyl ester, dichlorophenyl ester, tetrachlorophenyl ester, or 1-hydroxybenzotriazole ester; a triflate, mesylate, or tosylate; 2-ethyl-5-phenylisoxa-zolium-3′-sulfonate; a pyridyldisulfide, or nitropyridyldisulfide; a maleimide, haloacetate, acetylenedicarboxylic group, or carboxylic acid halogenate (fluoride, chloride, bromide, or iodide), or one of following structures: 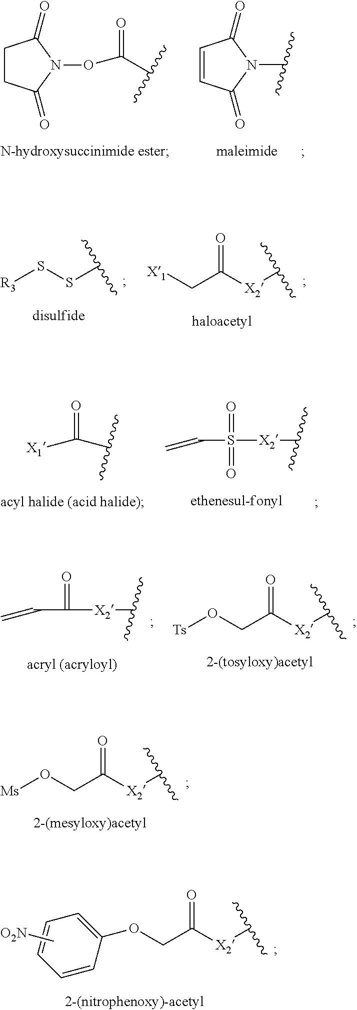 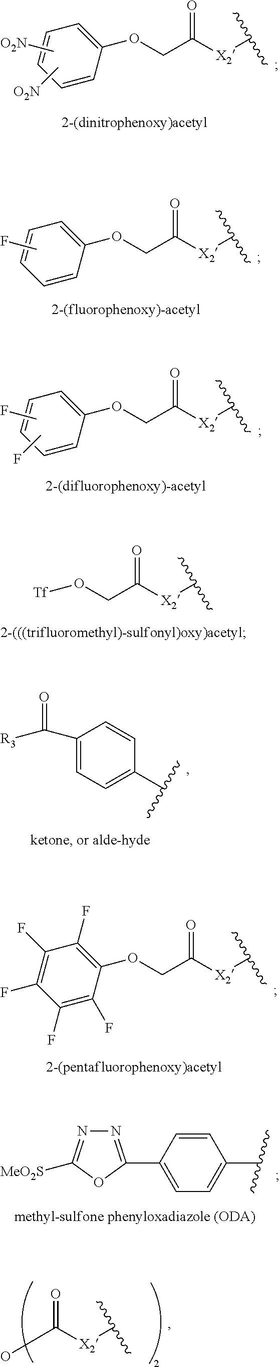 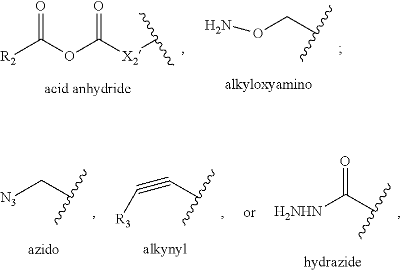
wherein X₁′ is F, Cl, Br, I or Lv₃; X₂′ is O, NH, N(R₁), or CH₂; R₃ and R₅ are H, R₁, aromatic, heteroaromatic, or aromatic group wherein one or several H atoms are replaced independently by —R₁, -halogen, —OR₁, —SR₁, —NR₁R₂, —NO₂, —S(O)R₁, —S(O)₂R₁, or —COOR₁; Lv₃ is a leaving group selected from methanesulfonyl, toluenesulfonyl, trifluoromethyl-sulfonyl, trifluoromethylsulfonate, nitrophenoxyl, N-succinimidyloxyl, phenoxyl; dinitrophenoxyl; pentafluorophenoxyl, tetrafluoro-phenoxyl, trifluorophenoxyl, difluorophenoxyl, monofluoro-phenoxyl, pentachlorophenoxyl, 1H-imidazole-1-yl, chlorophenoxyl, dichlorophenoxyl, trichlorophenoxyl, tetrachlorophenoxyl, N-(benzotriazol-yl)oxyl, 2-ethyl-5-phenylisoxazolium-yl, phenyloxadiazol-yl, oxadiazol-yl, or an intermediate molecule generated with a condensation reagent for Mitsunobu reactions, wherein R₁ and R₂ are defined above.

4. A bis-linker molecule of Formula (IV): 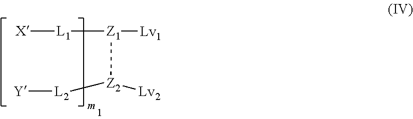
wherein
“ [symbol] ” represents a single bond; “ [symbol] ” is a single bond, a double bond, or absent; m₁ is 1 to 20;
Z₁ and Z₂ are, the same or different, and independently have one of following structures: C(O)CH, C(O)C, C(O)CH₂, ArCH₂, C(O), NH; NHNH; N(R₁); N(R₁)N(R₂); O; S; S—S, O—NH, O—N(R₁), CH₂—NH, CH₂—N(R₁), CH═NH, CH═N(R₁), S(O), S(O₂), P(O)(OH), S(O)NH, S(O₂)NH, P(O)(OH)NH, NHS(O)NH, NHS(O₂)NH, NHP(O)(OH)NH, N(R₁)S(O)N(R₂), N(R₁)S(O₂)N(R₂), N(R₁)P(O)(OH)N(R₂), OS(O)NH, OS(O₂)NH, OP(O)(OH)NH, C(O), C(NH), C(NR₁), C(O)NH, C(NH)NH, C(NR₁)NH, OC(O)NH, OC(NH)NH; OC(NR₁)NH, NHC(O)NH; NHC(NH)NH; NHC(NR₁)NH, C(O)NH, C(NH)NH, C(NR₁)NH, OC(O)C(R₁), OC(NH)N(R₁), OC(NR₁)N(R₁), NHC(O)N(R₁), NHC(NH)N(R₁), NHC(NR₁)N(R₁), N(R₁)C(O)N(R₁), N(R₁)C(NH)N(R₁), N(R₁)C(NR₁)N(R₁); or C₁-C₈ alkyl, C₂-C₈ heteroalkyl, alkylcycloalkyl, or heterocycloalkyl; and C₃-C₈ aryl, Ar-alkyl, heterocyclic, carbocyclic, cycloalkyl, heteroalkylcycloalkyl, alkylcarbonyl, or heteroaryl;
L₁ and L₂ are, the same or different, independently selected from O; NH; S; NHNH; N(R₃); N(R₃)N(R_{3′}); C₁-C₈ alkyl, amide, amines, imines, hydrazines, or hydrazones; C₂-C₈ heteroalkyl, alkylcycloalkyl, ethers, esters, hydrazones, ureas, semicarbazides, carbazides, alkoxyamines, alkoxylamines, urethanes, amino acids, peptides, acyloxylamines, hydroxamic acids, or heterocycloalkyl; C₃-C₈ aryl, Ar-alkyl, heterocyclic, carbocyclic, cycloalkyl, heteroalkylcycloalkyl, alkylcarbonyl, or heteroaryl; 1˜8 amino acids; and polyethyleneoxy unit of formula (OCH₂CH₂)ₚOR₃, or (OCH₂—CH(CH₃))ₚOR₃, or NH(CH₂CH₂O)ₚR₃, or NH(CH₂CH(CH₃)O)ₚR₃, or N[(CH₂CH₂O)ₚR₃]—[(CH₂CH₂O)_{p′}R_{3′}], or (OCH₂CH₂)ₚCOOR₃, or CH₂CH₂(OCH₂CH₂)ₚCOOR₃, wherein p and p′ are independently an integer selected from 0 to about 5000, or a combination thereof; R₃ and R_{3′} are independently H; C₁-C₈ alkyl; C₂-C₈ heteroalkyl, alkylcycloalkyl, or heterocycloalkyl, C₃-C₈ aryl, Ar-alkyl, heterocyclic, carbocyclic, cycloalkyl, heteroalkylcycloalkyl, oxy having formula (OCH₂CH₂)ₚ or (OCH₂—CH(CH₃))ₚ, wherein p is an integer from 0 to about 5000, or a combination thereof;
or L₁ and L₂ independently have one or more linker components of 6-maleimidocaproyl (“MC”), maleimidopropanoyl (“MP”), valine-citrulline (“val-cit” or “vc”), alanine-phenylalanine (“ala-phe” or “af”), p-aminobenzyloxycarbonyl (“PAB”), 4-thiopentanoate (“SPP”), 4-(N-maleimidomethyl)cyclohexane-1 carboxylate (“MCC”), (4-acetyl)amino-benzoate (“SIAB”), 4-thio-butyrate (SPDB), 4-thio-2-hydroxysulfonyl-butyrate (2-Sulfo-SPDB), or natural or unnatural peptide having 1˜8 natural or unnatural amino acid units;
or L₁ and L₂ independently contain a self-immolative component, peptidic unit, a hydrazone bond, a disulfide, an ester, an oxime, an amide, or a thioether bond: the self-immolative unit being an aromatic compound that is electronically similar to para-aminobenzyl-carbamoyl (PAB) groups, 2-aminoimidazol-5-methanol derivatives, heterocyclic PAB analogs, beta-glucuronide, and ortho or para-aminobenzylacetals; or one of following structures; 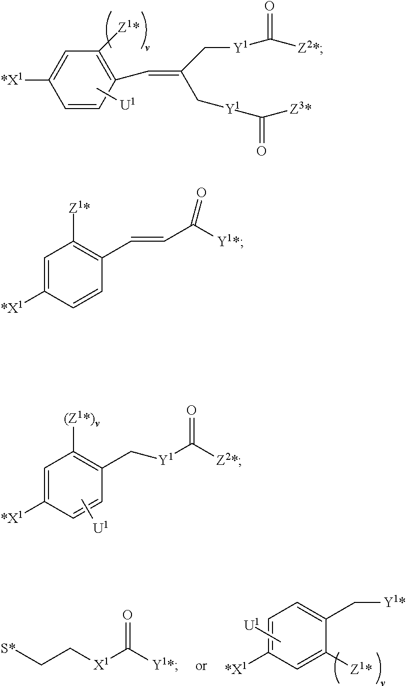
wherein the (*) atom is a point of attachment of additional spacer or releasable linker unit; X¹, Y¹, Z² and Z³ are independently NH, O, or S; Z¹ is independently H, NHR₁, OR₁, SR₁, or COX₁R₁, wherein X₁ and R₁ are defined above; v is 0 or 1; U¹ is independently H, OH, C₁˜C₆ alkyl, (OCH₂CH₂)ₙ, F, Cl, Br, I, OR₅, SR₅, NR₅R₅′, N═NR₅, NR₅, NR₅R₅′, NO₂, SOR₅R₅′, SO₂R₅, SO₃R₅, OSO₃R₅, PR₅R₅′, POR₅R₅′, PO₂R₅R₅′, OPO(OR₅)(OR₅′), or OCH₂PO(OR₅(OR₅′), wherein R₅ and R₅′ are independently selected from H, C₁˜C₈ alkyl; C₂˜C₈ alkenyl, alkynyl, heteroalkyl, or amino acid; C₃˜C₈ aryl, heterocyclic, carbocyclic, cycloalkyl, heterocycloalkyl, heteroaralkyl, alkylcarbonyl, or glycoside; or pharmaceutical cation salts;
or L₁ and L₂ independently have a non-self-immolative linker component containing one of following structures: 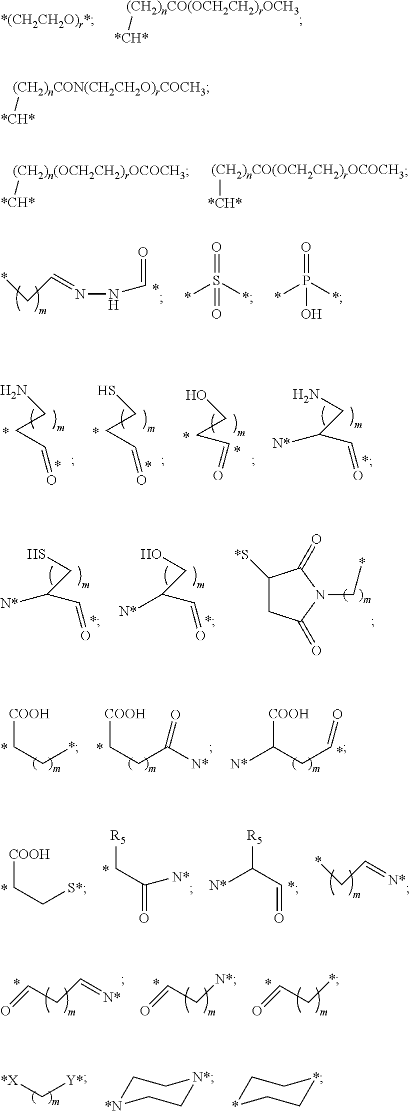 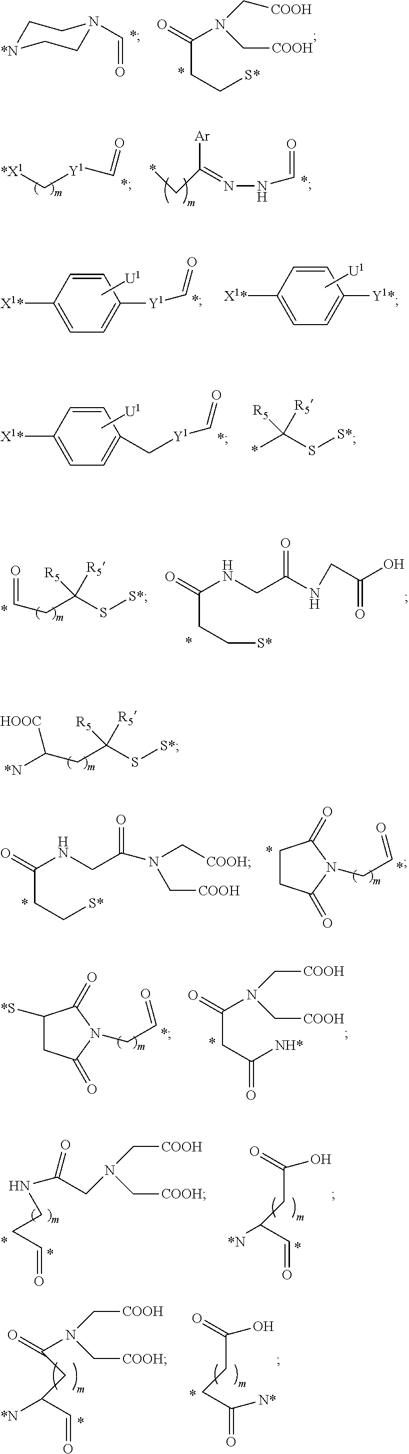 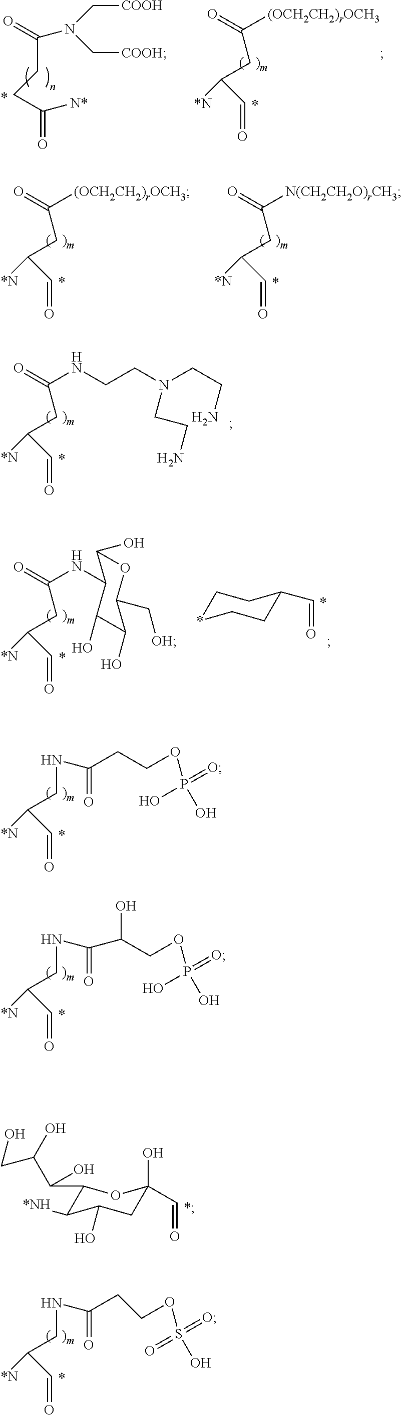 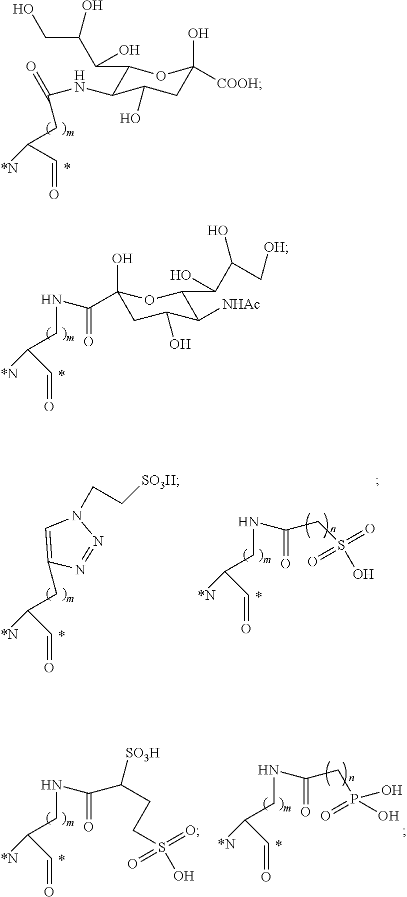
wherein the (*) atom is a point of attachment of additional spacer or releasable linker; X¹, Y¹, U¹, R₅, R₅′ are defined as above; r is 0˜100; m and n are 0˜6 independently;
or L₁ and L₂ independently are a releasable linker containing at least one bond that is capable of being broken under physiological conditions; a pH-labile, acid-labile, base-labile, oxidatively labile, metabolically labile, biochemically labile or enzyme-labile bond, having one of following structures: —(CR₅R₆)ₘ(Aa)ᵣ(CR₇R₈)ₙ(OCH₂CH₂)ₜ—, —(CR₅R₆)ₘ(CR₇R₈)ₙ(Aa)ᵣ(OCH₂CH₂)ₜ—, - (Aa)ₜ-(CR₅R₆)ₘ(CR₇R₈)ₙ(OCH₂CH₂)ₜ—, —(CR₅R₆)ₘ(CR₇R₈)ₙ(OCH₂CH₂)ᵣ(Aa)ₜ-, —(CR₅R₆)ₘ—(CR₇═CR₈)(CR₉R₁₀)ₙ(Aa)ₜ(OCH₂CH₂)ᵣ—, —(CR₅R₆)ₘ(NR₁₁CO)(Aa)ₜ(CR₉R₁₀)ₙ—(OCH₂CH₂)ᵣ—, —(CR₅R₆)ₘ(Aa)t(NR₁₁CO)(CR₉R₁₀)ₙ(OCH₂CH₂)ₜ—, —(CR₅R₆)ₘ(OCO)(Aa)ₜ(CR₉R₁₀)ₙ—(OCH₂CH₂)ₜ—, —(CR₅R₆)ₘ(OCNR₇)(Aa)ᵣ(CR₉R₁₀)ₙ(OCH₂CH₂)ᵣ—, —(CR₅R₆)ₘ(CO)(Aa)ₜ-(CR₉R₁₀)ₙ(OCH₂CH₂)ᵣ—, —(CR₅R₆)ₘ(NR₁₁CO)(Aa)ᵣ(CR₉R₁₀)ₙ(OCH₂CH₂)ᵣ—, —(CR₅R₆)ₘ—(OCO)(Aa)ₜ(CR₉R₁₀)ₙ—(OCH₂CH₂)ᵣ—, —(CR₅R₆)ₘ(OCNR₇)(Aa)t(CR₉R₁₀)ₙ(OCH₂CH₂)ᵣ—, —(CR₅R₆)ₘ(CO)(Aa)ᵣ(CR₉R₁₀)ₙ—(OCH₂CH₂)ᵣ—, —(CR₅R₆)ₘ-phenyl-CO)(Aa)ₜ(CR₇R₈)ₙ—, —(CR₅R₆)ₘ-furyl-CO(Aa)ₜ(CR₇R₈)ₙ—, —(CR₅R₆)ₘ-oxazolyl-CO(Aa)ᵣ(CR₇R₈)ₙ—, —(CR₅R₆)ₘ-thiazolyl-CO(Aa)ₜ(CCR₇R₈)ₙ—, —(CR₅R₆)ₜ-thienyl-CO(CR₇R₈)ₙ—, —(CR₅R₆)ₜ-imidazolyl-CO—(CR₇R₈)ₙ—, —(CR₅R₆)ₜ-morpholino-CO(Aa)ₜ-(CR₇R₈)ₙ—, —(CR₅R₆)ₜpiperazino-CO(Aa)ₜ-(CR₇R₈)ₙ—, —(CR₅R₆)ₜ—N-methylpiperazin-CO(Aa)ₜ-(CR₇R₈)ₙ—, —(CR₅R₆)ₘ-(Aa)ₜphenyl-, —(CR₅R₆)ₘ-(Aa)ₜfuryl-, —(CR₅R₆)ₘ-oxazolyl(Aa)ₜ-, —(CR₅R₆)ₘ-thiazolyl(Aa)ᵣ-, —(CR₅R₆)ₘ-thienyl-(Aa)ₜ-, —(CR₅R₆)ₘ-imidazolyl(Aa)ₜ-, —(CR₅R₆)ₘ-morpholino-(Aa)ₜ-, —(CR₅R₆)ₘ-piperazino-(Aa)ₜ-, —(CR₅R₆)ₘ—N-methylpiperazino-(Aa)ₜ-, —K(CR₅R₆)ₘ(Aa)ᵣ(CR₇R₈)ₙ(OCH₂CH₂)ₜ—, —K(CR₅R₆)ₘ(CR₇R₈)ₙ(Aa)ᵣ(OCH₂CH₂)ₜ—, —K(Aa)ₜ-(CR₅R₆)ₘ(CR₇R₈)ₙ(OCH₂CH₂)ₜ—, —K(CR₅R₆)ₘ(CR₇R₈)ₙ(OCH₂CH₂)t(Aa)ₜ-, —K(CR₅R₆)ₘ—(CR₇═CR₈)(CR₉R₁₀)ₙ(Aa)ₜ(OCH₂CH₂)ᵣ—, —K(CR₅R₆)ₘ(NR₁₁CO)(Aa)ₜ(CR₉R₁₀)ₙ(OCH₂CH₂)ᵣ—, —K(CR₅R₆)ₘ(Aa)t(NR₁₁CO)(CR₉R₁₀)ₙ(OCH₂CH₂)ₜ—, —K(CR₅R₆)ₘ(OCO)(Aa)ₜ(CR₉R₁₀)ₙ—(OCH₂CH₂)ᵣ—, —K(CR₅R₆)ₘ(OCNR₇)(Aa)ₜ(CR₉R₁₀)ₙ(OCH₂CH₂)ₜ—, —K(CR₅R₆)ₘ(CO)(Aa)ₜ-(CR₉R₁₀)ₙ(OCH₂CH₂)ᵣ—, —K(CR₅R₆)ₘ(NR₁₁CO)(Aa)ₜ(CR₉R₁₀)ₙ(OCH₂CH₂)ᵣ—, —K(CR₅R₆)ₘ—(OCO)(Aa)ₜ(CR₉R₁₀)ₙ(OCH₂CH₂)ᵣ—, —K(CR₅R₆)ₘ(OCNR₇)(Aa)ₜ(CR₉R₁₀)ₙ(OCH₂CH₂)ᵣ—, —K—(CR₅R₆)ₘ(CO)(Aa)ₜ(CR₉R₁₀)ₙ(OCH₂CH₂)ᵣ—, —K(CR₅R₆)ₘ-phenyl-CO(Aa)ₜ(CR₇R₈)ₙ—, —K—(CR₅R₆)ₘ-furyl-CO(Aa)ₜ-(CR₇R₈)ₙ—, —K(CR₅R₆)ₘ-oxazolyl-CO(Aa)ₜ(CR₇R₈)ₙ—, —K(CR₅R₆)ₘ-thiazolyl-CO(Aa)ₜ-(CR₇R₈)ₙ—, —K(CR₅R₆)ₜ-thienyl-CO(CR₇R₈)ₙ—, —K(CR₅R₆)t imidazolyl-CO—(CR₇R₈)ₙ—, —K(CR₅R₆)t morpholino-CO(Aa)ₜ(CR₇R₈)ₙ—, —K(CR₅R₆)t piperazino-CO(Aa)ₜ-(CR₇R₈)n-, —K(CR₅R₆)ₜ—N-methylpiperazinCO(Aa)ₜ(CR₇R₈)ₙ—, —K(CR₅R₆)ₘ(Aa)ₜphenyl, —K—(CR₅R₆)ₘ; (Aa)ₜfuryl-, —K(CR₅R₆)ₘ-oxazolyl(Aa)ₜ-, —K(CR₅R₆)ₘ-thiazolyl(Aa)ₜ-, —K(CR₅R₆)ₘ-thienyl-(Aa)ₜ-, —K(CR₅R₆)ₘ-imidazolyl(Aa)ₜ-, —K(CR₅R₆)ₘ-morpholino(Aa)ᵣ-, —K(CR₅R₆)ₘ-piperazino-(Aa)ₜG, —K(CR₅R₆)ₘN-methylpiperazino(Aa)ₜ-; wherein m, Aa, m, and n are described above; t and r are 0-100 independently; R₃, R₄, R₅, R₆, R₇, and R₈ are independently H; halide; C₁˜C₈ alkyl; C₂˜C₈ aryl, alkenyl, alkynyl, ether, ester, amine or amide, which optionally substituted by one or more halide, CN, NR₁R₂, CF₃, OR₁, Aryl, heterocycle, S(O)R₁, SO₂R₁, —CO₂H, —SO₃H, —OR₁, —CO₂R₁, —CONR₁, —PO₂R₁R₂, —PO₃H or P(O)R₁R₂R₃; K is NR₁, —SS—, —S(═O)—, —C(═O)NH—, —C(═O))—, —C═NH—O—, —C═N—NH—, —C(═O)NH—NH—, O, S, Se, B, Het (heterocyclic or heteroaromatic ring having C₃-C₈), or peptide containing 1-20 amino acids;
or L₁ and L₂ independently contain one of following hydrophilic structures: 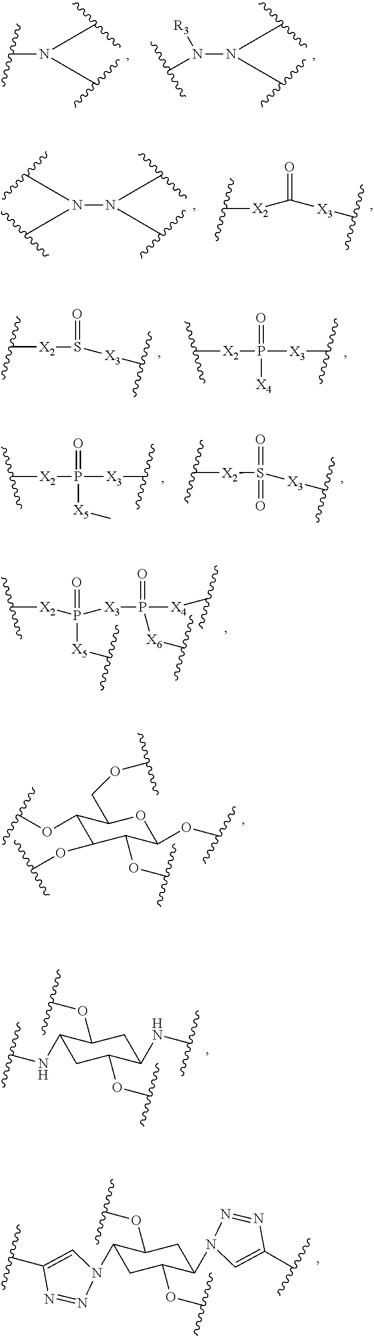 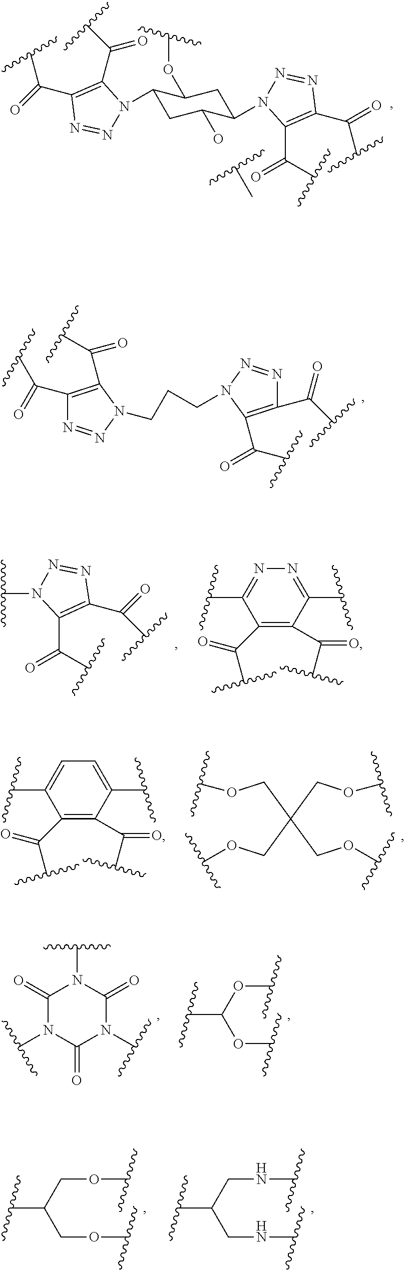 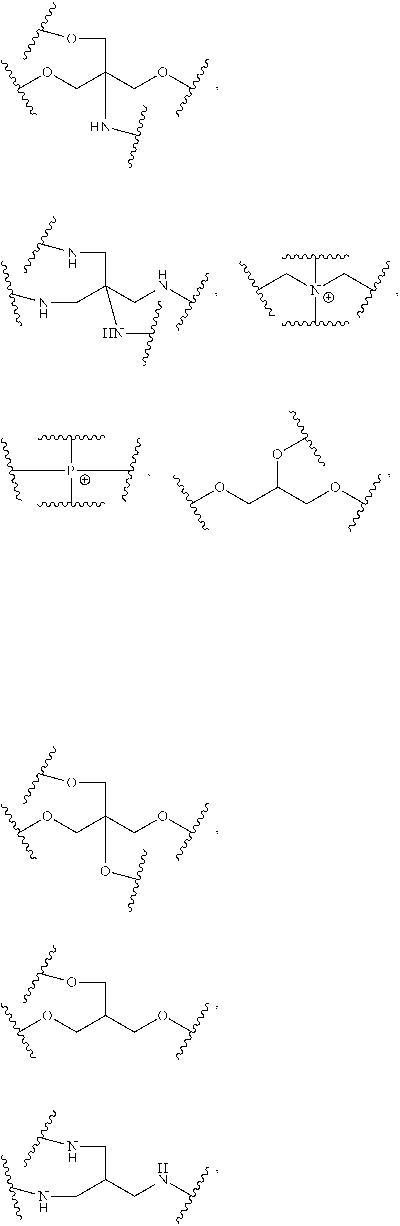 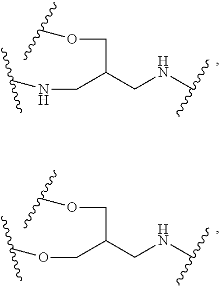
wherein [symbol] is a site of linkage; X₂, X₃, X₄, X₅, or X₆ are independently NH; NHNH; N(R₃); N(R₃)N(R_{3′}); O; S; C₁-C₆ alkyl; C₂-C₆ heteroalkyl, alkylcycloalkyl, or heterocycloalkyl, C₃-C₈ aryl, Ar-alkyl, heterocyclic, carbocyclic, cycloalkyl, heteroalkylcycloalkyl, alkylcarbonyl, or heteroaryl; or 1˜8 amino acids; wherein R₃ and R_{3′} are independently H; C1-C₈ alkyl; C2-C₈ hetero-alkyl, alkylcycloalkyl, or heterocycloalkyl, C₃-C₈ aryl, Ar-alkyl, heterocyclic, carbocyclic, cycloalkyl, heteroalkylcycloalkyl, alkylcarbonyl, or heteroaryl; or C₂-C₈ ester, ether or amide; or amide; or polyethyleneoxy unit having formula (OCH₂CH₂)ₚ or (OCH₂CH(CH₃))ₚ, wherein p is an integer from 0 to about 5000, or a combination thereof;
or L₁, L₂, Z₁ or Z₂ are independently composed of one or more following components: 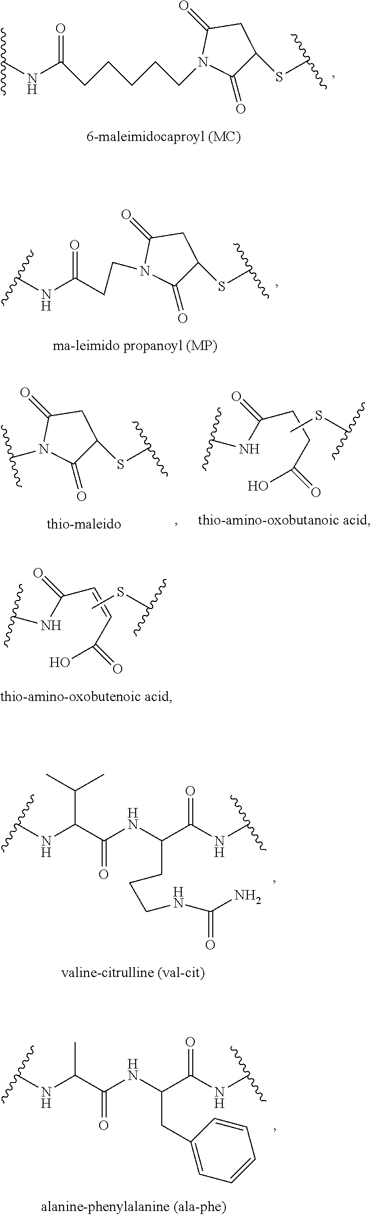 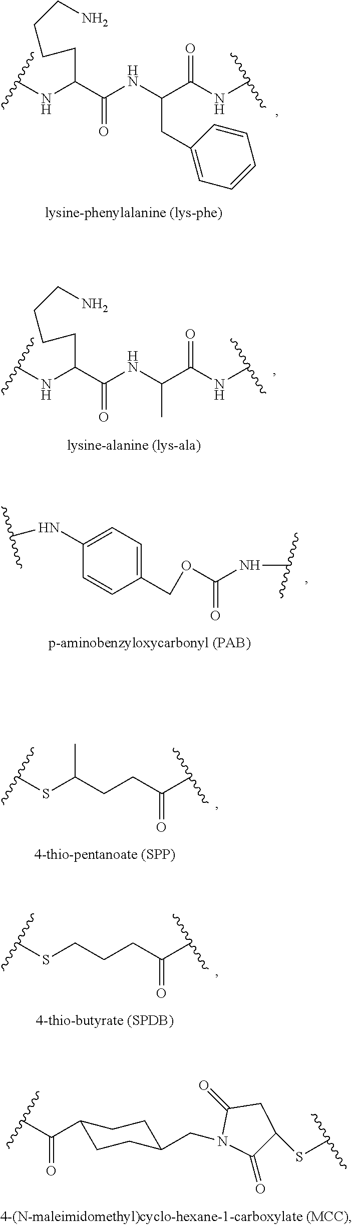 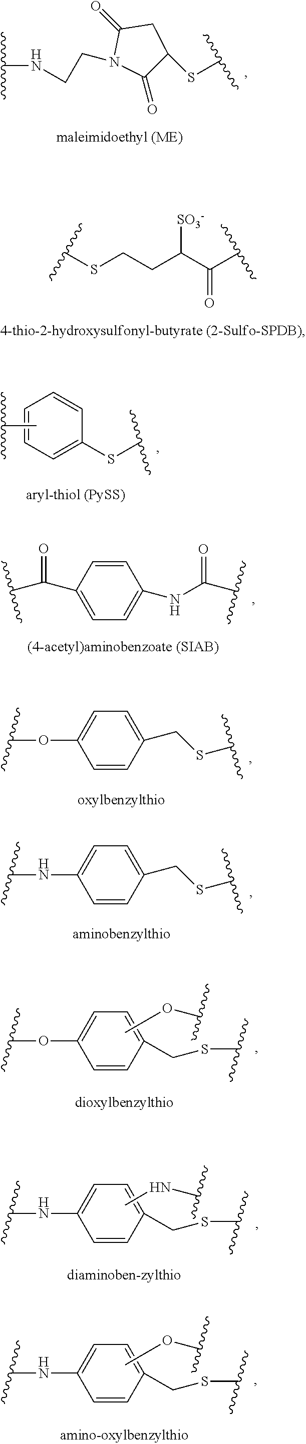 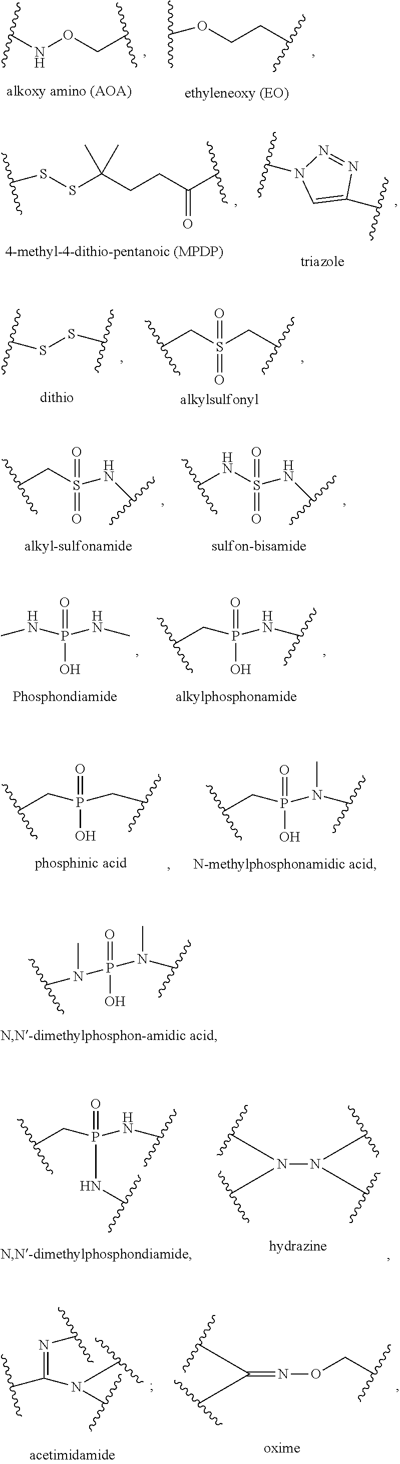 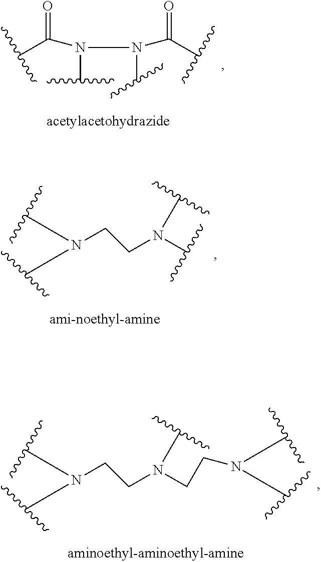 [SMILES: *C(=O)N(*)N(*)C(*)=O ; *N(*)CCN(*)* ; *N(*)CCN(*)CCN(*)*]
and L- or D-, natural or unnatural peptides containing 1-20 amino acids;
wherein [symbol] is a site that another bond is connected to;
or L₁, L₂, Z₁, or Z₂ are independently absent, provided that L₁ and Z₁, L₂ and Z₂ are not absent at the same time;
Lv₁ and Lv₂ represent the same or different leaving group that is capable of reacting with a thiol, amine, carboxylic acid, selenol, phenol or hydroxyl group on a cell-binding molecule; Lv₁ and Lv₂ are independently selected from OH; F; Cl; Br; I; nitrophenol; N-hydroxysuccinimide; phenol; dinitrophenol; pentafluorophenol; tetrafluorophenol; difluorophenol; mono-fluorophenol; pentachlorophenol; triflate; imidaole; dichlorophenol; tetrachlorophenol; 1-hydroxybenzotriazole; tosylate; mesylate; 2-ethyl-5-phenylisoxazolium-3′-sulfonate, anhydrides formed its self, or formed with the other anhydride: acetyl anhydride, or formyl anhydride, or an intermediate molecule generated with a condensation reagent for peptide coupling reactions, or for Mitsunobu reactions, which are selected from EDC (N-(3-Dimethylaminopropyl)-N′-ethylcarbodiimide), Dicyclohexyl-carbodiimide, N,N′-Diisopropylcarbodiimide, N-Cyclohexyl-N′-(2-morpholino-ethyl)carbodiimide metho-n-toluenesulfonate, 1,1′-Carbonyldiimi-dazole, O-(Benzotriazol-1-yl)-N,N,N′,N′-tetramethyluronium tetrafluoroborate, N,N,N′,N′-Tetramethyl-O-(1H-benzotriazol-1-yl)-uronium hexafluorophosphate, (Benzotriazol-1-yloxy)tris(dimethylamino)-phosphonium hexafluorophosphate, (Benzotriazol-1-yloxy)tripyrrolidinophosphonium hexafluoro-phosphate, Diethyl cyanophosphonate, Chloro-N,N,N′,N′-tetramethylformamidiniumhexafluorophosphate, 1-[Bis(dimethylamino)methylene]-1H-1,2,3-triazolo[4,5-b])pyridinium 3-oxid hexafluorophos-phate, 1-[Dimethylamino)(morpholino)methylene]-1H-[1,2,3]triazolo[4,5 b]pyridine-1-ium 3-oxide hexafluoro-phosphate, 2-Chloro-1,3-dimethyl-imidazolidinium hexafluorophosphate, Chlorotripyrrolidinophosphonium, hexafluoro-phosphate, Fluoro-N,N,N′,N′-bis(tetramethylene)-formamidinium hexafluorophosphate, N,N,N′,N′-Tetramethyl-S-(1-oxido-2-pyridyl)thiuronium hexafluorophosphate, O-(2-Oxo-1(2H)pyridyl)-N,N,N′,N′-tetramethyl-uronium tetrafluoroborate, S-(1-Oxido-2-pyridyl)-N,N,N′,N′-tetramethylthiuronium tetrafluoroborate, O-[(Ethoxycarbonyl)-cyanomethylenamino]-N,N,N′,N′-tetramethyluronium hexafluorophosphate, (1-Cyano-2-ethoxy-2-oxoethylidenaminooxy) dimethyl-amino-morpholino-carbenium hexafluorophosphate, O-(Benzotriazol-1-yl)-N,N,N′,N′-bis(tetramethylene)uronium hexafluorophosphate, N-Benzyl-N′-cyclohexyl-carbodiimide (with, or without polymer-bound), Dipyrrolidino)N-succinimidyl-oxylcarbenium hexafluoro-phosphate, Chlorodipyrrolidinocarbenium hexafluorophosphate, 2-Chloro-1,3-dimethylimidazolidinium tetrafluoroborate, (Benzotriazol-1-yloxy)dipiperi-dinocarbenium hexafluorophosphate, O-(6)-Chlorobenzotriazol-1-yl)-N,N,N′,N′-tetramethyluronium tetrafluoroborate, Bromo-tris(dimethylamino-phosphonium hexafluorophosphate, Propylphosphonic anhydride, 2-Morpholinoethyl isocyanide, N,N,N′,N′-Tetramethyl-O—(N-succinimidyl)uronium hexafluorophosphate, 2-Bromo-1-ethyl-pyridinium tetrafluoroborate, O-[Ethoxycarbonyl)cyano-methylenamino]-N,N,N′,N′-tetra-methyluronium tetrafluoroborate, 4-(4,6-Dimethoxy-1,3,5-triazin-2-yl)-4-methylmorpholiniumchloride, N,N,N′,N′-Tetramethyl-O—(N-succinimidyl)uronium tetrafluoroborate, O-(3,4-Dihydro-4-oxo-1,2,3-benzotriazin-3-yl-N,N,N′,N′-tetramethyluronium tetrafluoro-borate, 1,1′-(Azodicarbonyl)-dipiperidine, Di-(4-chlorobenzyl)azodicarboxylate, Di-tert-butyl azodicarboxylate, Diisopropyl azodicarboxylate, Diethyl azodicarboxylate, or Lv₁ and Lv₂ are an anhydride, formed by acid themselves or formed with other C1˜C8 acid anhydrides;
or Lv₁ and Lv₂ are independently, a halide, methanesulfonyl, toluenesulfonyl, trifluoromethyl-sulfonyl, trifluoromethylsulfonate, nitrophenoxyl, N-succinimidyloxyl, phenoxyl: dinitrophenoxyl; pentafluorophenoxyl, tetrafluorophenoxyl, trifluorophenoxyl, difluorophenoxyl, monofluorophenoxyl, pentachlorophenoxyl, 1H-imidazole-1-yl, chlorophenoxyl, dichlorophenoxyl, trichlorophenoxyl, tetrachlorophenoxyl, N-(benzotriazol-yl)oxyl, 2-ethyl-5-phenylisoxazolium-3′-sulfonyl, phenyloxadiazole-sulfonyl (-sulfone-ODA), 2-ethyl-5-phenylisoxazolium-yl, phenyloxadiazol-yl, oxadiazol-yl, unsaturated carbon (a double or a triple bond between carbon-carbon, carbon-nitrogen, carbon-sulfur, carbon-phosphorus, sulfur-nitrogen, phosphorus-nitrogen, oxygen-nitrogen, or car-bon-oxygen), or one of following structures: 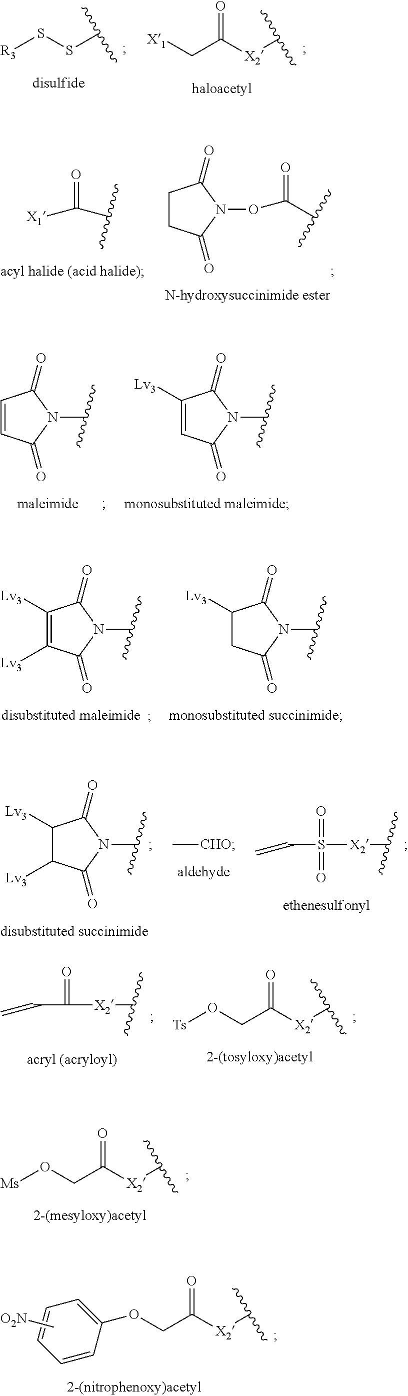 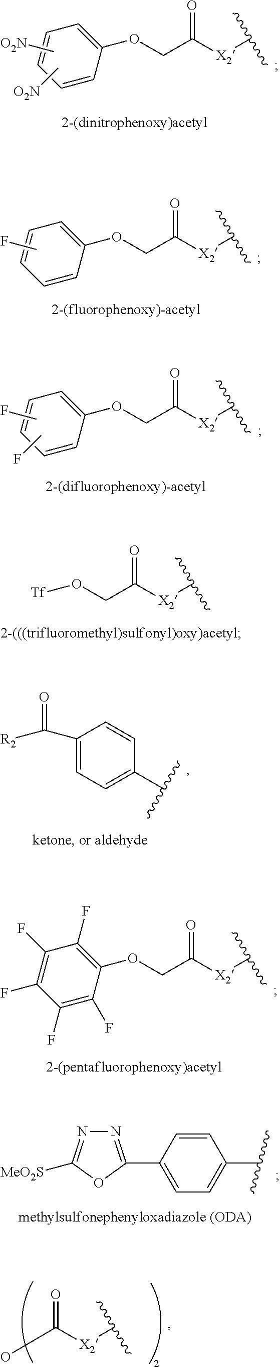 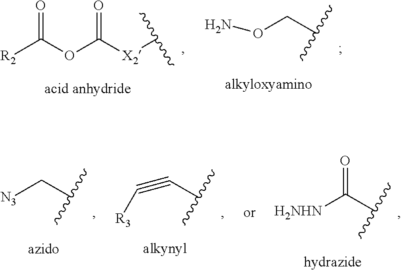
wherein for X₁′ is F, Cl, Br, I or Lv₃; X₂′ is O, NH, N(R₁), or CH₂; R₃ is independently H, aromatic, heteroaromatic or aromatic group wherein one or several H atoms are replaced independently by —R₁, -halogen, —OR₁, —SR₁, —NR₁R₂, —NO₂, —S(O)R₁, —S(O)₂R₁, or —COOR₁; Lv₃ is a leaving group selected from F, Cl, Br, I, nitrophenol; N-hydroxysuccinimide (NHS); phenol; dinitrophenol; pentafluorophenol, tetrafluorophenol, difluorophenol; monofluorophenol, pentachlorophenol; triflate; imidazole; dichlorophenol; tetrachlorophenol, 1-hydroxybenzotriazole, tosylate; mesylate; 2-ethyl-5-phenylisoxazolium-3′-sulfonate; R₁ and R₂ are independently H, C₁-C₈ alkyl, C₂-C₈ alkenyl, heteroalkyl, alkylcycloalkyl, or heterocycloalkyl; C₃-C₈ aryl, Ar-alkyl, heterocyclic, carbocyclic, cycloalkyl, heteroalkylcycloalkyl, alkylcarbonyl, or heteroaryl, or C₂-C₈ ester, ether, or amide; or peptide containing 1-8 amino acids; or polyethyleneoxy unit having formula (OCH₂CH₂)ₚ, or (OCH₂CH(CH₃))ₚ wherein p is an integer from 0 to about 5000, or a combination thereof; and
X′ and Y′ are a function group that is capable of independently reacting with a residue gram of a cytotoxic drug simultaneously or sequentially;
X′ and Y′ are independently a disulfide substituent maleimido haloacetyl, alkoxyamine azido, ketone, aldehyde, hydrazine amino, hydroxyl, carboxylate, imidazole, thiol or alkyne; or a N-hydroxysuccinimide ester, p-nitrophenyl ester, dinitrophenyl ester, pentafluorophenyl ester, pentachlorophenyl ester; tetrafluorophenyl ester, difluorophenyl ester; monofluorophenyl ester; or pentachlorophenyl ester, dichlorophenyl ester, tetrachlorophenyl ester, or 1-hydroxybenzotriazole ester, a triflate, mesylate, or tosylate, 2-ethyl-5-phenylisoxa-zolium-3′-sulfonate; a pyridyldisulfide, or nitropyridyldisulfide, a maleimide, haloacetate, acetylenedicarboxylic group, or carboxylic acid halogenate (fluoride, chloride, bromide, or iodide), or one of the following structures: 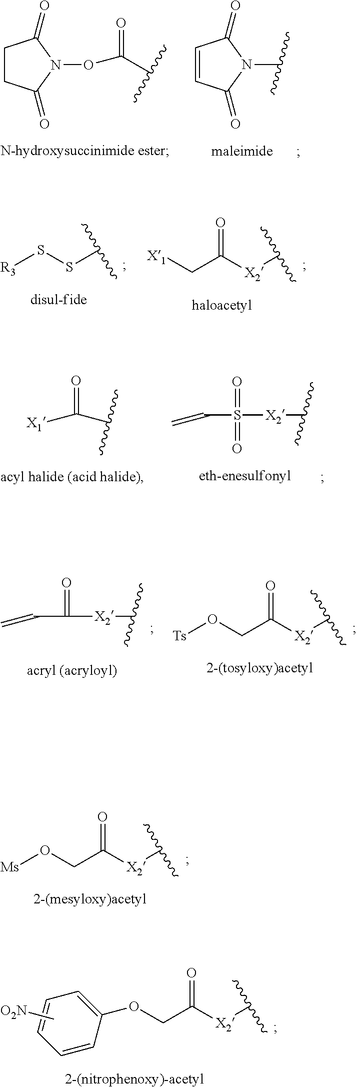 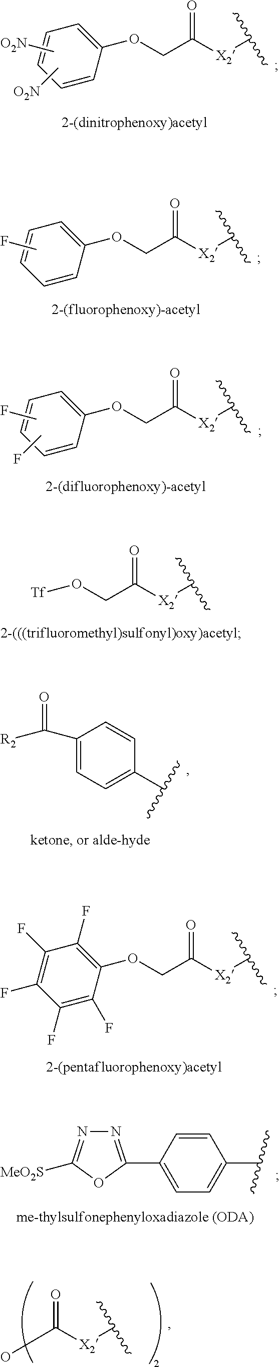 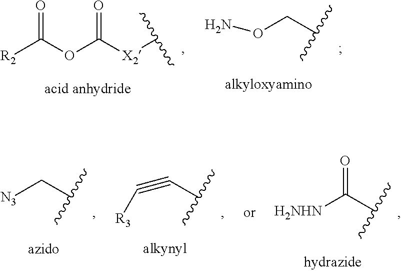
wherein X₁′ is F, Cl, Br, I or Lv₃; X₂′ is O, NH, N(R₁) or CH₂; R₃ and R₅ are H, R₁ aromatic, heteroaromatic, or aromatic group wherein one or several H atoms are replaced independently by —R₁, -halogen, —OR₁, —SR₁, NR₁R₂, —NO₂, —S(O)R₁, —S(O)₂R₃, or —COOR₁; Lv₃ is a leaving group selected front methanesulfonyl, toluenesulfonyl, trifluoromethyl-sulfonyl, trifluoromethylsulfonate, nitrophenoxyl, N-succinimidyloxyl, phenoxyl, dinitrophenoxyl; pentafluorophenoxyl, tetrafluoro-phenoxyl, trifluorophenoxyl, difluorophenoxyl, monofluoro-phenoxyl, pentachlorophenoxyl, 1H-imidazole-1-yl, chlorophenoxyl, dichlorophenoxyl, trichlorophenoxyl, tetrachlorophenoxyl, N-(benzotriazol-yl)oxyl, 2-ethyl-5-phenylisoxazolium-yl, phenyloxadiazol-yl, oxadiazol-yl, or an intermediate molecule generated with a condensation reagent for Mitsunobu reactions, wherein R₁ and R₂ are defined above;
wherein the bis-linker molecule of Formula (IV) excludes following structure: 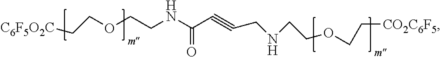
wherein m″=1-3.

5. The conjugate compound of Formula (I) of claim 1 having a structure represented by Formula (I-a), (I-b), (I-c), (I-d), (I-e), (I-f), (I-g), (I-h), (I-i), (I-j), (I-k), (I-m), (I-n), (I-o), (I-p), (I-q), (I-r), (I-s), (I-t), (I-u), (I-v), or (I-w) below: 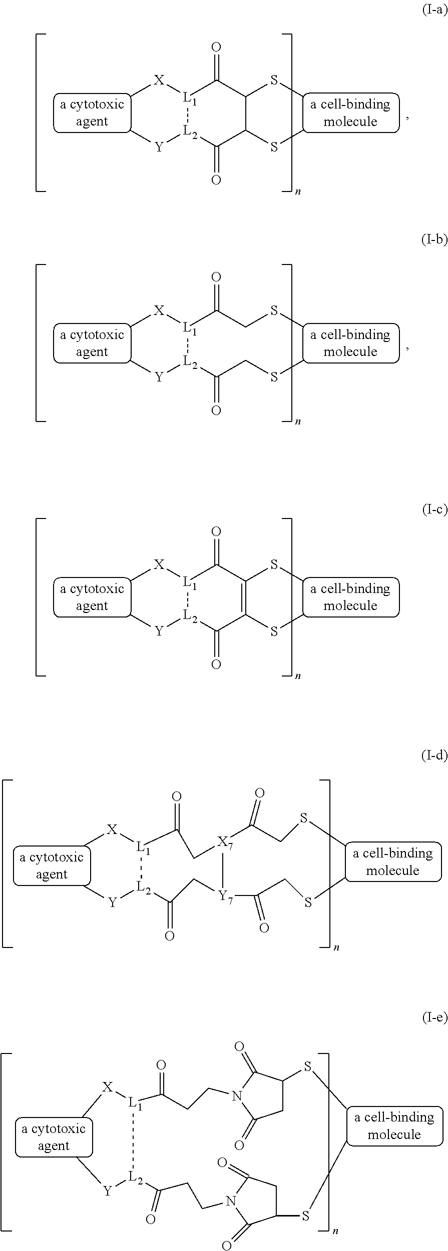 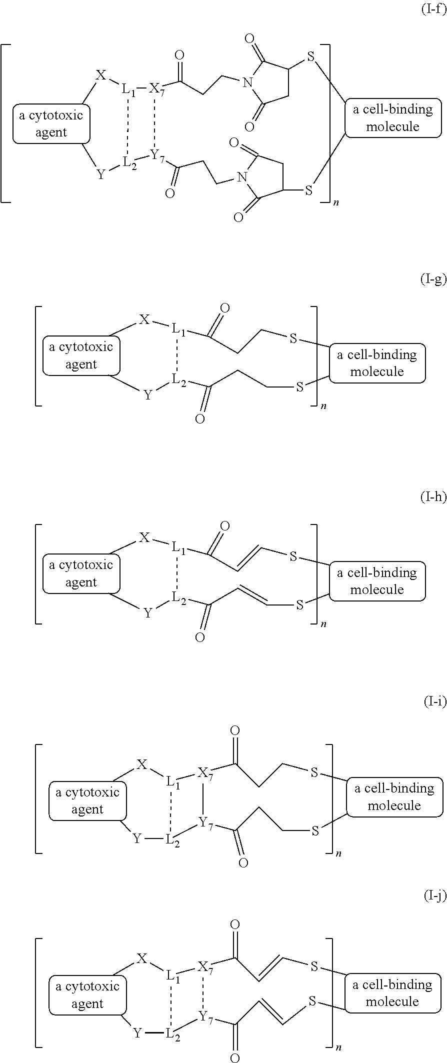 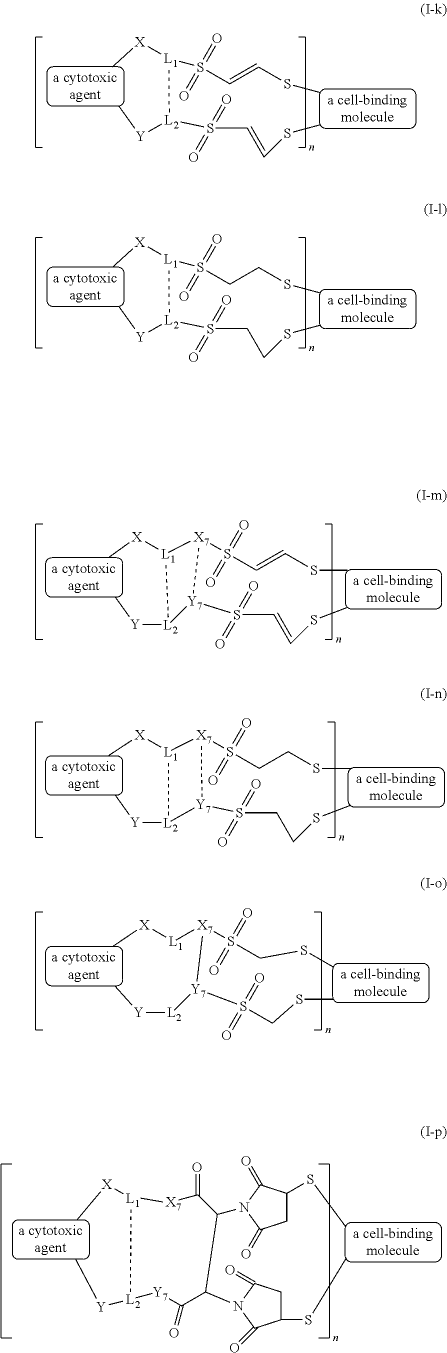 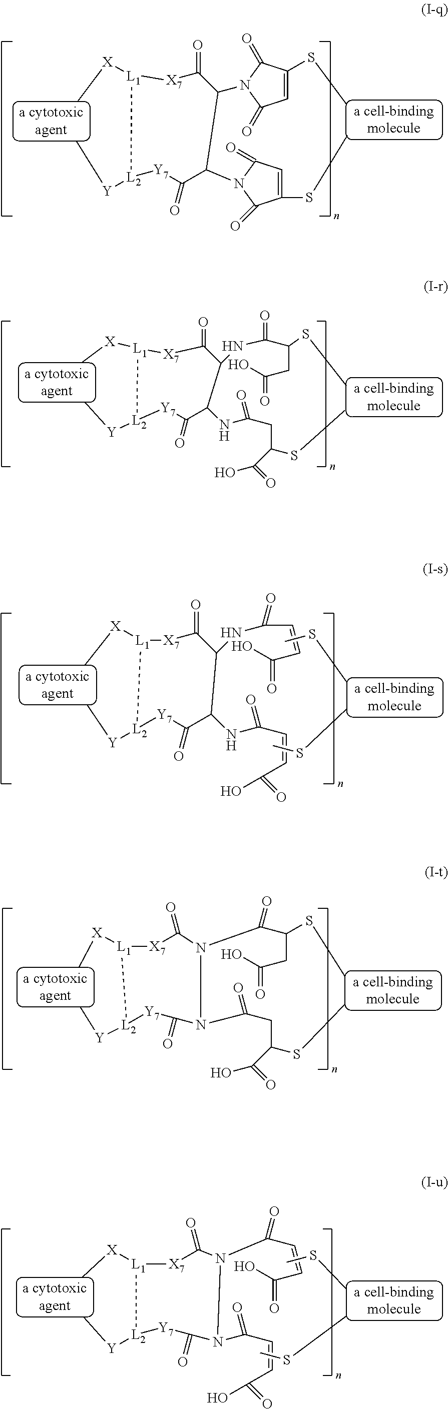 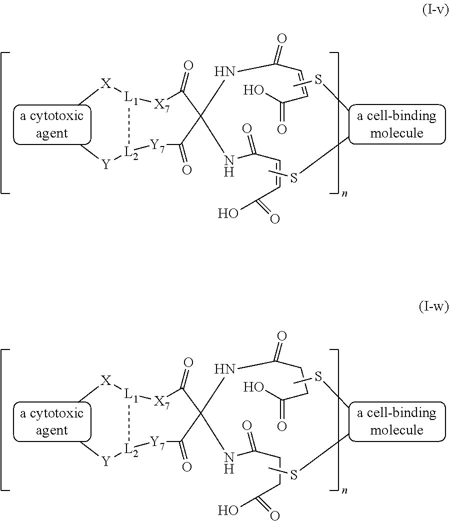
wherein X₇ and Y₇ are independently CH, CH₂, NH, O, S, NHNH, N(R₁), or N; “-----”, X, Y, R₁, n, L₁, L₂, a cell-binding, molecule and a cytotoxic agent are defined the same as in claim 1.

6. The compound of Formula (II) of claim 2 having a structure represented by Formula (II-a), (II-b), (II-c), (II-d), (II-e), (II-f), (II-g), (II-h), (II-i), (II-i), (II-k), (II-m), (II-n), (II-o), (II-q), (II-r), (II-s), (II-t), (II-u), (II-v), (II-w), (II-x), (II-y), (II-z), (II-a1), (II-a2), (II-a3), or (II-a4): 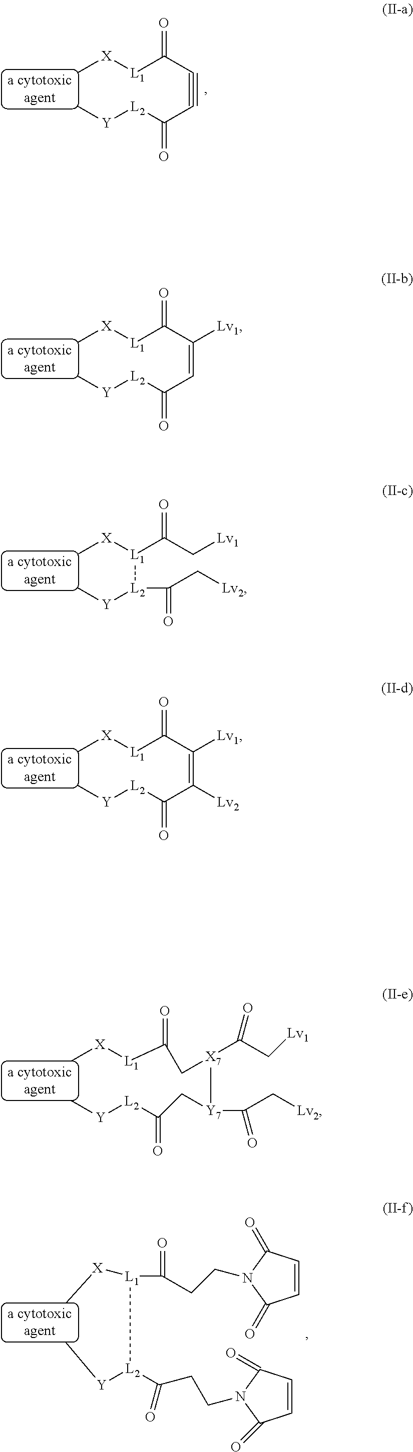 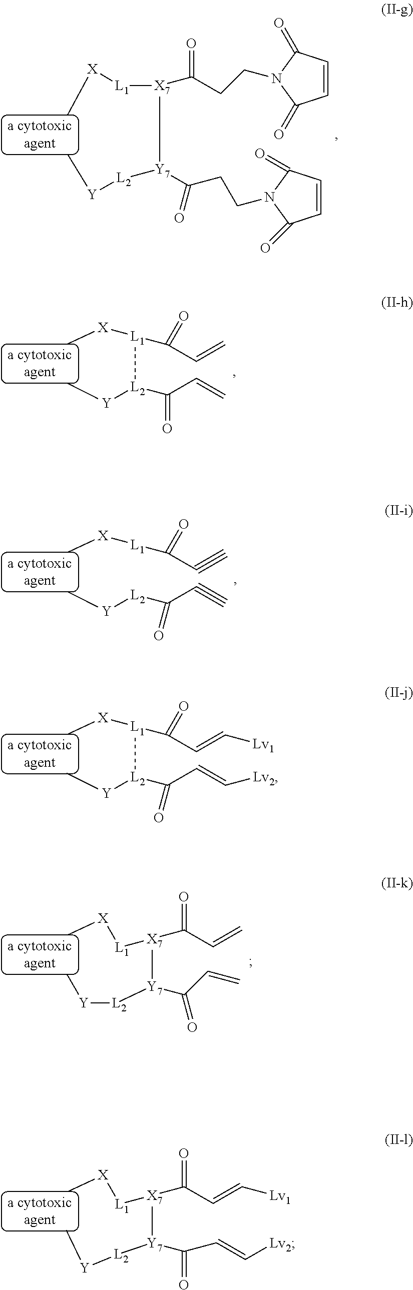 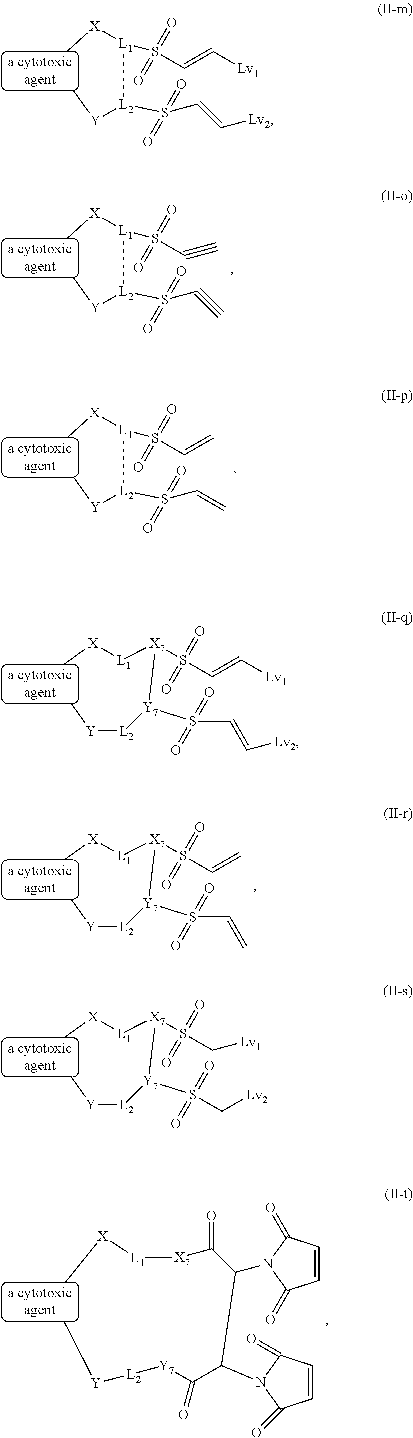 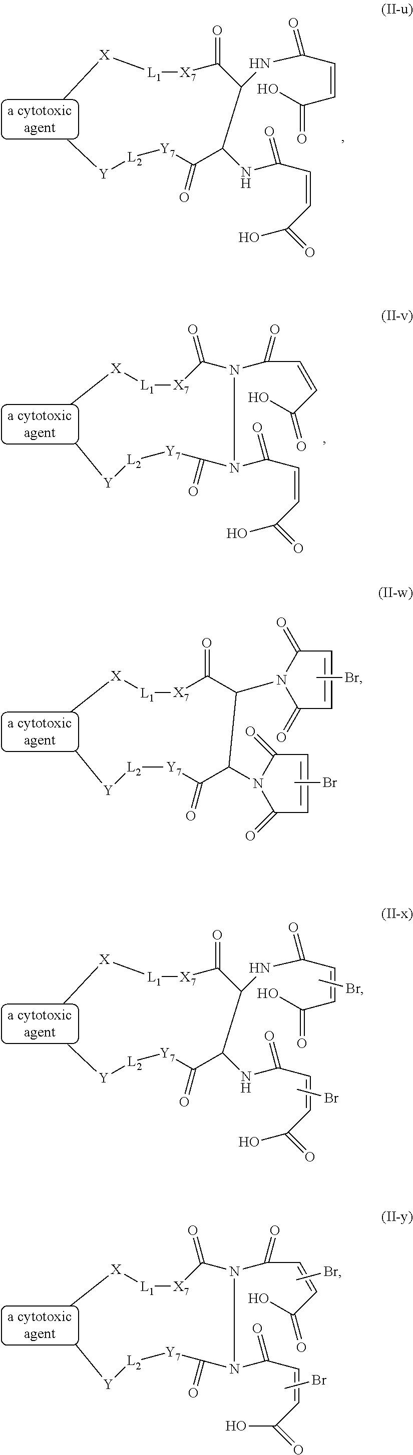 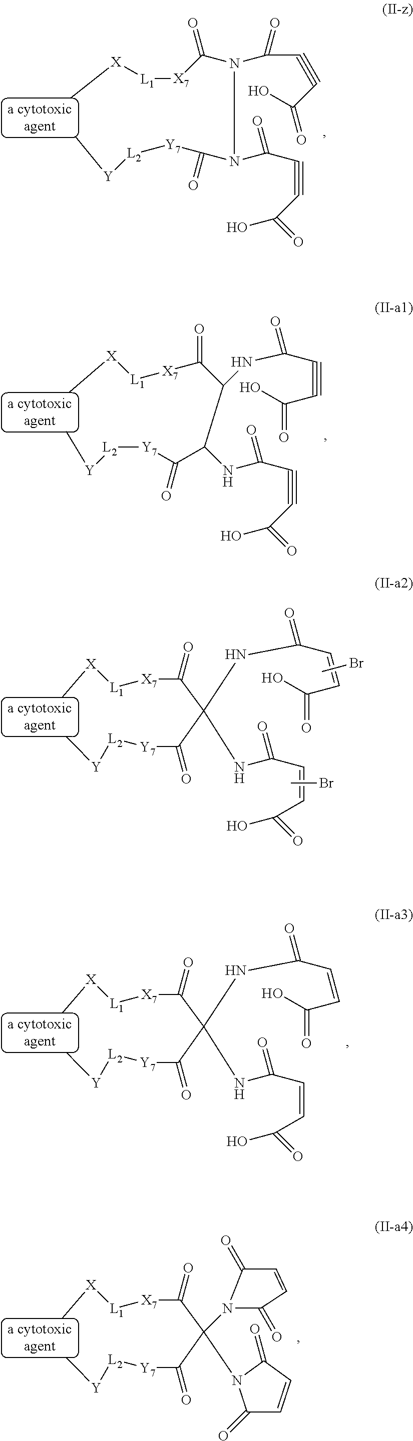
wherein X₇ and Y₇ are independently CH, —CH₂, NH, O, S, NHNH, N(R₁), or N; “-----”, cytotoxic agent, R₁, X, Y, n, L₁, L₂, Lv₁ and Lv₂ are described the same as in claim 1.

7. The bis-linker of Formula (III) of claim 3 having a structure represented by Formula (III-a), (III-b), (III-c), (III-d), (III-e), (III-f), (III-g), (III-h), (III-i), (III-j), (III-k), (III-l), (III-m), (III-n), (III-o), (III-p), (III-r), (III-s), (III-t), (III-u), (III-v), or (III-w) below: 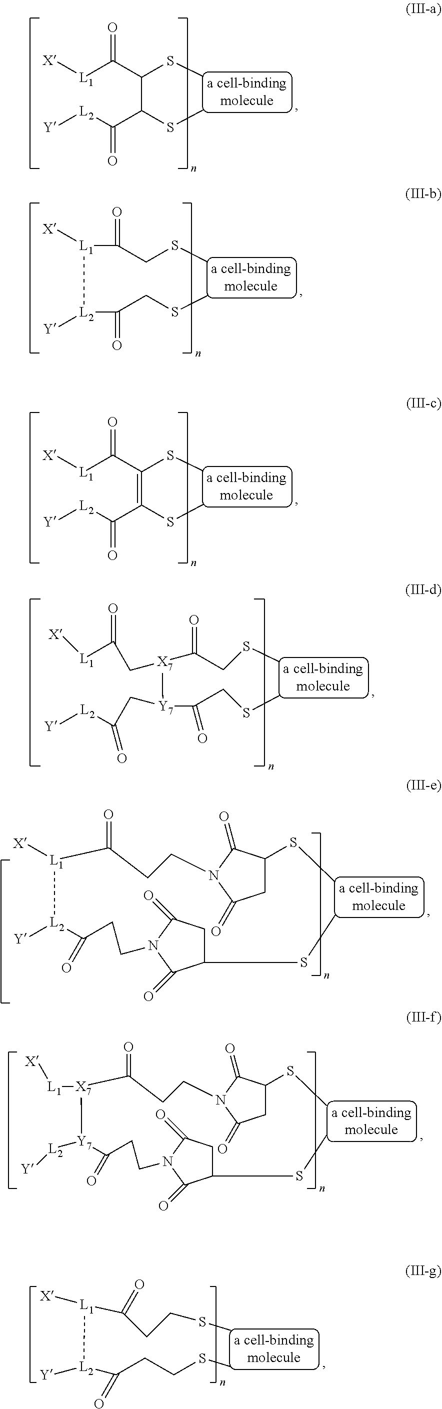 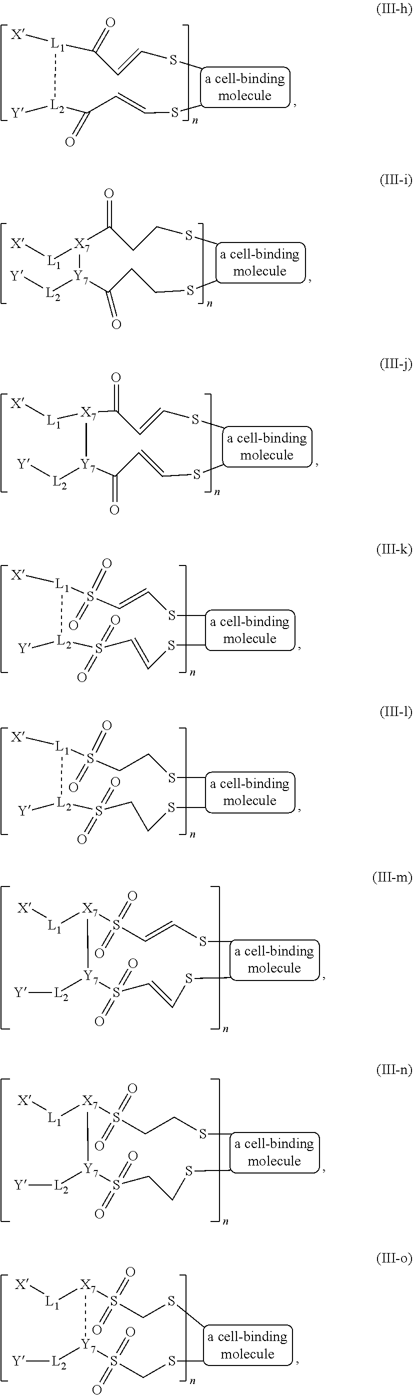 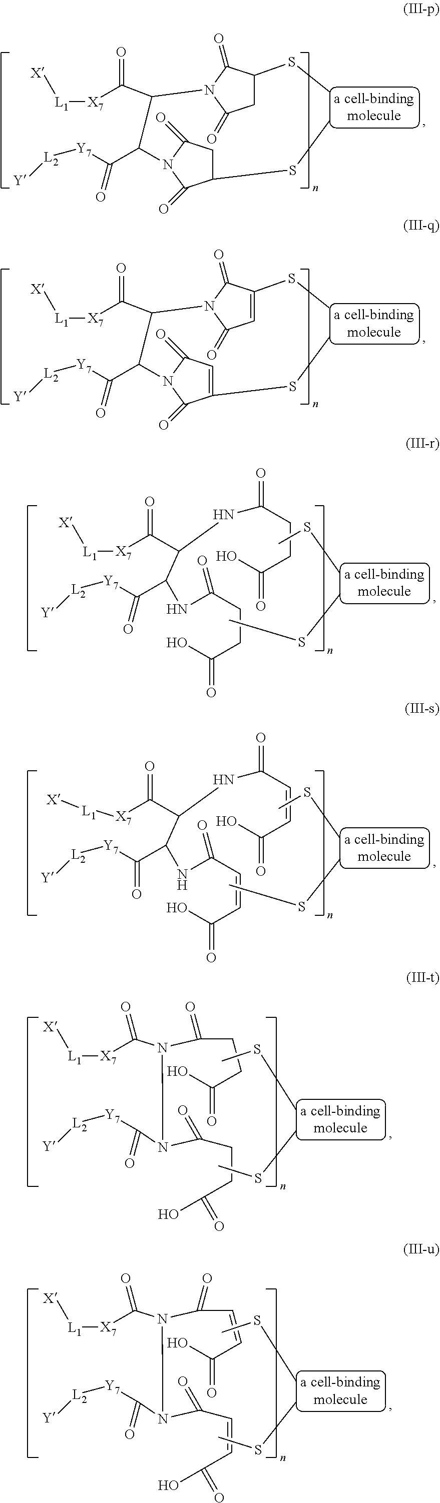 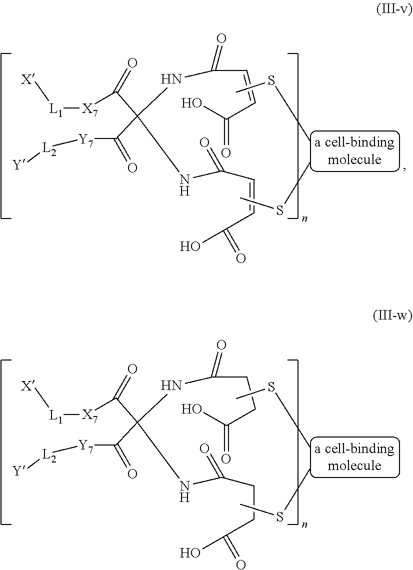
wherein X₇ and Y₇ are independently CH, CH₂, NH, O, S, NHNH, N(R₁), or N; a cell-binding molecule, R₁, X′, Y′, n, L₁ and L₂ are defined the same as in claim 3.

8. The bis-linker molecule of Formula (IV) of claim 4 having a structure represented by Formula (IV-a), (IV-b), (IV-c), (IV-d), (IV-e), (IV-f), (IV-g), (IV-h), (IV-i), (IV-j), (IV-k), (IV-m), (IV-n), (IV-o), (IV-p), (IV-q), (IV-r), (IV-s), (IV-t), (IV-u), (IV-v), (IV-w), (IV-x), (IV-y), (IV-z), (IV-a1), (IV-a2), (IV-a3), or (IV-a4) below: 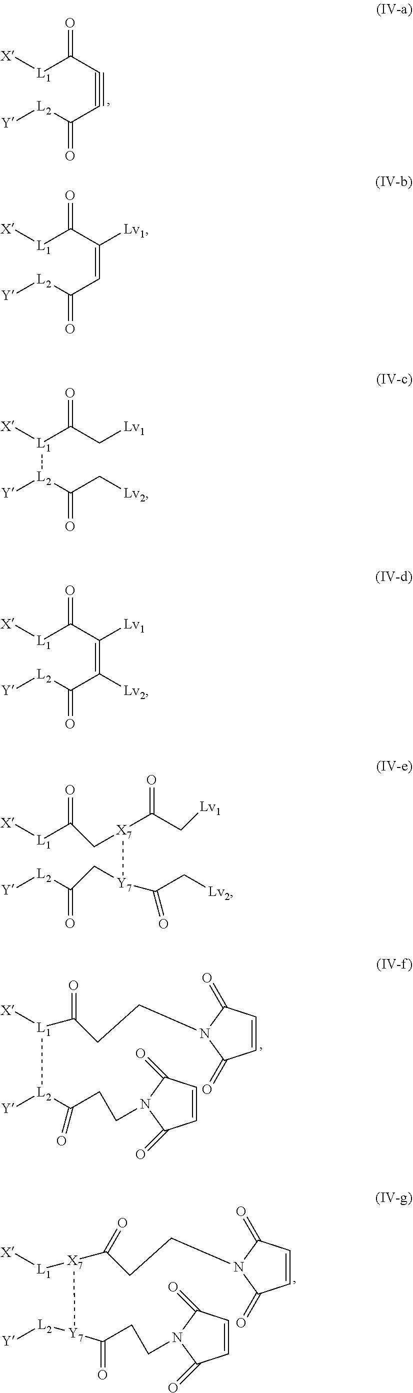 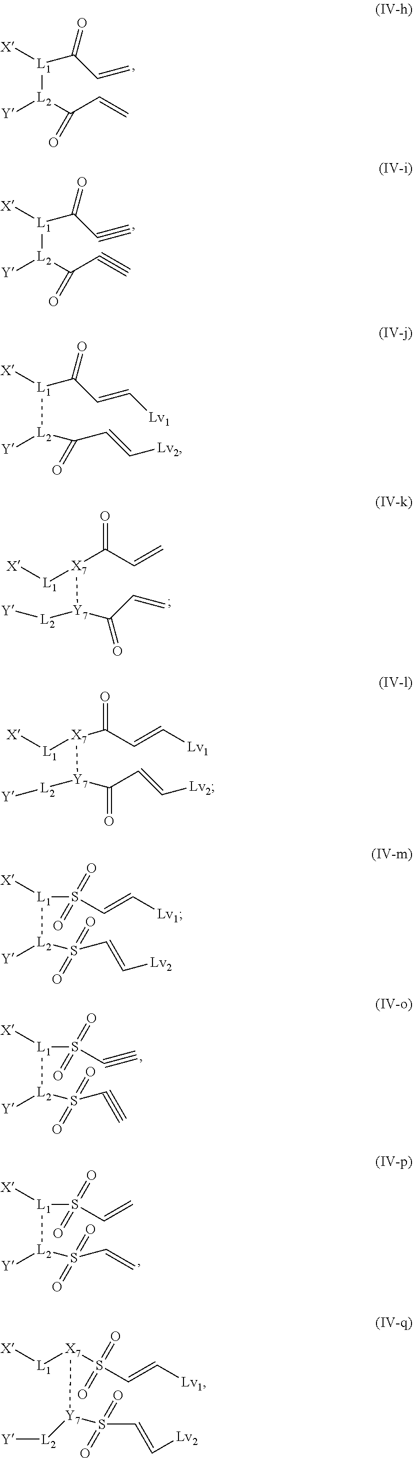 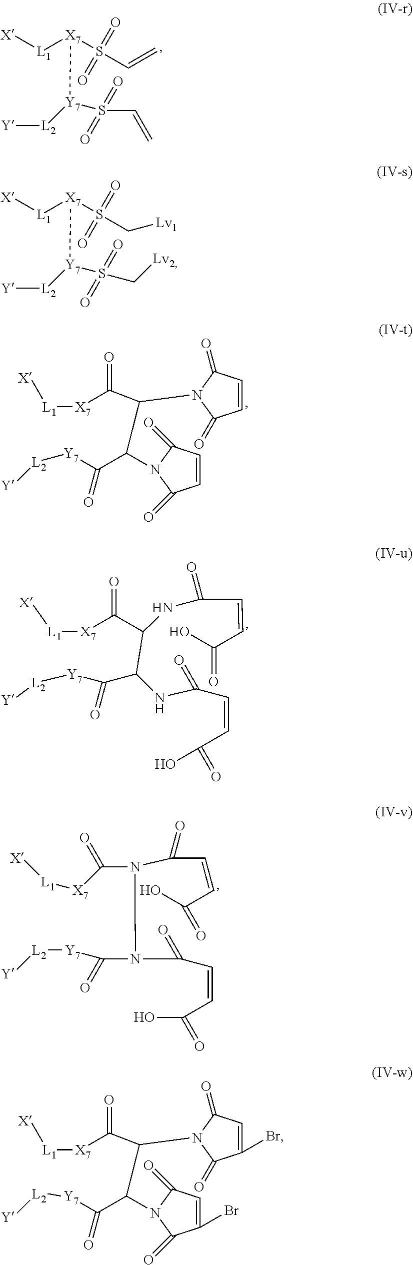 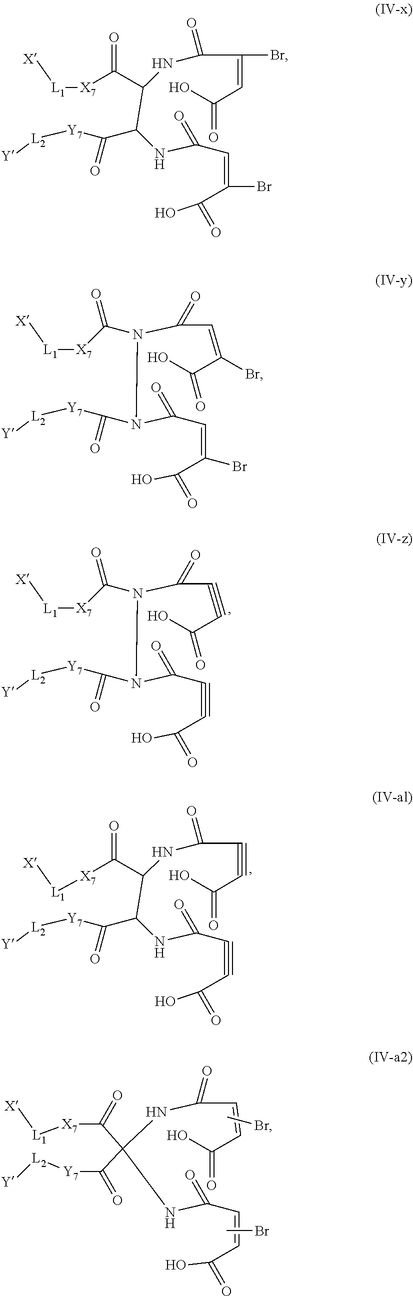 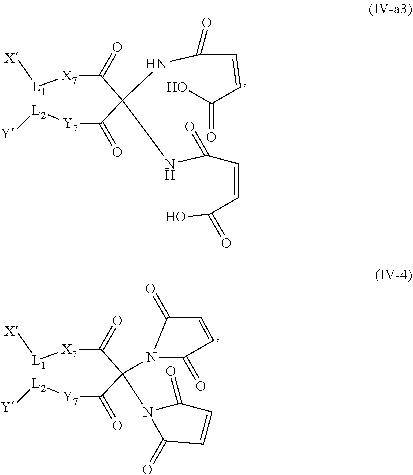
wherein X₇ and Y₇ are independently CH, CH₂, NH, O, S, NHNH, N(R₁), or N; “-----”, R₁, X′, Y′, n, L₁ and L₂ are defined the same as in claim 4.

9. The conjugate compound according to claim 1, wherein the cell-binding molecule is connected to Z₁ and Z₂ via a pair of thiols formed from inter chain disulfide atoms of the cell-binding molecule.

10. The conjugate compound according to claim 1, wherein the cytotoxic molecule is selected from:
(1). a chemotherapeutic agent selected from the group consisting of:
a). an alkylating agent: selected from the group consisting of nitrogen mustards: chlorambucil, chlornaphazine, cyclophosphamide, dacarbazine, estramustine, ifosfamide, mechlorethamine, mechlorethamine oxide hydrochloride, mannomustine, mitobronitol, melphalan, mitolactol, pipobroman, novembichin, phenesterine, prednimustine, thiotepa, trofosfamide, uracil mustard; CC-1065 and adozelesin, carzelesin, bizelesin or their synthetic analogues; duocarmycin and its synthetic analogues, KW-2189, CBI-TMI, or CBI dimers; benzodiazepine dimers or pyrrolobenzodiazepine (PBD) dimers, tomaymycin dimers, indolinobenzodiazepine dimers, imidazobenzothiadiazepine dimers, or oxazolidinobenzodiazepine dimers; Nitrosoureas: comprising carmustine, lomustine, chlorozotocin, fotemustine, nimustine, ranimustine; Alkylsulphonates: comprising busulfan, treosulfan, improsulfan and piposulfan); Triazenes or dacarbazine; Platinum containing compounds: comprising carboplatin, cisplatin, and oxaliplatin; aziridines, benzodopa, carboquone, meturedopa, or uredopa; ethylenimines and methylamelamines including altretamine, triethylenemel-amine, trietylenephosphoramide, triethylenethiophosphoramide and trimethylolomelamine];
b). a plant alkaloid: selected from the group consisting of Vinca alkaloids: comprising vincristine, vinblastine, vindesine, vinorelbine, and navelbin; Taxoids: comprising paclitaxel, docetaxol and their analogs, Maytansinoids comprising DM1, DM2, DM3, DM4, DM5, DM6, DM7, maytansine, ansamitocins and their analogs, cryptophycins (including the group consisting of cryptophycin 1 and cryptophycin 8); epothilones, eleutherobin, discodermolide, bryostatins, dolostatins, auristatins, tubulysins, cephalostatins; pancratistatin; a sarcodictyin; spongistatin;
c). a DNA Topoisomerase inhibitor: selected from the groups of Epipodophyllins: comprising 9-aminocamptothecin, camptothecin, crisnatol, daunomycin, etoposide, etoposide phosphate, irinotecan, mitoxantrone, novantrone, retinoic acids (or retinols), teniposide, topotecan, 9-nitrocamptothecin or RFS 2000; and mitomycins and their analogs;
d). an antimetabolite: selected from the group consisting of {[Anti-folate: (DHFR inhibitors: comprising methotrexate, trimetrexate, denopterin, pteropterin, aminopterin (4-aminopteroic acid) or folic acid analogues); IMP dehydrogenase Inhibitors: (comprising mycophenolic acid, tiazofurin, ribavirin, EICAR); Ribonucleotide reductase Inhibitors: (comprising hydroxyurea, deferoxamine)]; [Pyrimidine analogs: Uracil analogs: (comprising ancitabine, azacitidine, 6-azauridine, capecitabine (Xeloda), carmofur, cytarabine, dideoxyuridine, doxifluridine, enocitabine, 5-Fluorouracil, floxuridine, ratitrexed (Tomudex)); Cytosine analogs: (comprising cytarabine, cytosine arabinoside, fludarabine); Purine analogs: (comprising azathioprine, fludarabine, mercaptopurine, thiamiprine, thioguanine)]; folic acid replenisher, frolinic acid};
e). a hormonal therapy: selected from the group consisting of {Receptor antagonists: [Anti-estrogen: (comprising megestrol, raloxifene, tamoxifen); LHRH agonists: (comprising goscrclin, leuprolide acetate); Anti-androgens: (comprising bicalutamide, flutamide, calusterone, dromostanolone propionate, epitiostanol, goserelin, leuprolide, mepitiostane, nilutamide, testolactone, trilostane and other androgens inhibitors)]; Retinoids/Deltoids: [Vitamin D3 analogs: (comprising CB 1093, EB 1089 KH 1060, cholecalciferol, ergocalciferol); Photodynamic therapies: (comprising verteporfin, phthalocyanine, photosensitizer Pc4, demethoxyhypocrellin A); Cytokines: (comprising Interferon-alpha, Interferon-gamma, tumor necrosis factor (TNFs), human proteins containing a TNF domain)]};
f). a kinase inhibitor, selected from the group consisting of BIBW 2992 (anti-EGFR/Erb2), imatinib, gefitinib, pegaptanib, sorafenib, dasatinib, sunitinib, erlotinib, nilotinib, lapatinib, axitinib, pazopanib, vandetanib, E7080 (anti-VEGFR2), mubritinib, ponatinib (AP24534), bafetinib (INNO-406), bosutinib (SKI-606), cabozantinib, vismodegib, iniparib, ruxolitinib, CYT387, axitinib, tivozanib, sorafenib, bevacizumab, cetuximab, Trastuzumab, Ranibizumab, Panitumumab, and ispinesib;
g). a poly (ADP-ribose) polymerase (PARP) inhibitor selected from the group consisting of olaparib, niraparib, iniparib, talazoparib, veliparib, CEP 9722 (Cephalon's), E7016 (Eisai's), BGB-290 (BeiGene's), and 3-aminobenzamide;
h). an antibiotic, selected from the group consisting of an enediyne antibiotic (selected from the group consisting of calicheamicin, calicheamicin γ1, δ1, α1 or β1; dynemicin, including dynemicin A and deoxydynemicin; esperamicin, kedarcidin, C-1027, maduropeptin, or neocarzinostatin chromophore and related chromoprotein enediyne antibiotic chromomophores), aclacinomycins, actinomycin, authramycin, azaserine, bleomycins, cactinomycin, carabicin, carminomycin, carzinophilin; chromomycins, dactinomycin, daunorubicin, detorubicin, 6-diazo-5-oxo-L-norleucine, doxorubicin, morpholino-doxorubicin, cyanomorpholino-doxorubicin, 2-pyrrolino-doxorubicin and deoxydoxorubicin, epirubicin, eribulin, esorubicin, idarubicin, marcellomycin, nitomycins, mycophenolic acid, nogalamycin, olivomycins, peplomycin, potfiromycin, puromycin, quelamycin, rodorubicin, streptonigrin, streptozocin, tubercidin, ubenimex, zinostatin, and zorubicin;
i). a polyketide (acetogenin), bullatacin and bullatacinone; gemcitabine, epoxomicins and -carfilzomib, bortezomib, thalidomide, lenalidomide, pomalidomide, tosedostat, zybrestat, PLX4032, STA-9090, Stimuvax, allovectin-7, Xegeva, Provenge, Yervoy, Isoprenylation inhibitors and Lovastatin, Dopaminergic neurotoxins and 1-methyl-4-phenylpyridinium ion, Cell cycle inhibitors (selected from staurosporine), Actinomycins (comprising Actinomycin D, dactinomycin), amanitins, Bleomycins (comprising bleomycin A2, bleomycin B2, peplomycin), Anthracyclines (comprising daunorubicin, doxorubicin (adriamycin), idarubicin, epirubicin, pirarubicin, zorubicin, mtoxantrone, MDR inhibitors or verapamil, Ca²⁺ ATPase inhibitors or thapsigargin, Histone deacetylase inhibitors ((comprising Vorinostat, Romidepsin, Panobinostat, Valproic acid, Mocetinostat (MGCD0103), Belinostat, PCI-24781, Entinostat, SB939, Resminostat, Givinostat, AR-42, CUDC-101, sulforaphane, Trichostatin A); Thapsigargin, Celecoxib, glitazones, epigallocatechin gallate, Disulfiram, Salinosporamide A; Anti-adrenals, selected from the group consisting of aminoglutethimide, mitotane, trilostane; aceglatone; aldophosphamide glycoside; aminolevulinic acid; amsacrine; arabinoside, bestrabucil; bisantrene; edatraxate; defofamine; demecolcine; diaziquone; eflornithine (DFMO), elfomithine; elliptinium acetate, etoglucid; gallium nitrate; gacytosine, hydroxyurea; ibandronate, lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; rhizoxin; sizofiran; spirogermanium; tenuazonic acid; triaziquone; 2, 2′,2″-trichlorotriethylamine; trichothecenes (including the group consisting of T-2 toxin, verrucarin A, roridin A and anguidine); urethane, siRNA, antisense drugs;
(2). an anti-autoimmune disease agent: cyclosporine, cyclosporine A, aminocaproic acid, azathioprine, bromocriptine, chlorambucil, chloroquine, cyclophosphamide, corticosteroids (including the group consisting of amcinonide, betamethasone, budesonide, hydrocortisone, flunisolide, fluticasone propionate, fluocortolone danazol, dexamethasone, Triamcinolone acetonide, beclometasone dipropionate), DHEA, enanercept, hydroxychloroquine, infliximab, meloxicam, methotrexate, mofetil, mycophenylate, prednisone, sirolimus, tacrolimus;
(3). an anti-infectious disease agents comprising:
a). aminoglycosides: amikacin, astromicin, gentamicin (netilmicin, sisomicin, isepamicin), hygromycin B, kanamycin (amikacin, arbekacin, bekanamycin, dibekacin, tobramycin), neomycin (framycetin, paromomycin, ribostamycin), netilmicin, spectinomycin, streptomycin, tobramycin, verdamicin;
b). amphenicols: azidamfenicol, chloramphenicol, florfenicol, thiamphenicol;
c). ansamycins: geldanamycin, herbimycin;
d). carbapenems: biapenem, doripenem, ertapenem, imipenem/cilastatin, meropenem, panipenem;
e). cephems: carbacephem (loracarbef), cefacetrile, cefaclor, cefradine, cefadroxil, cefalonium, cefaloridine, cefalotin or cefalothin, cefalexin, cefaloglycin, cefamandole, cefapirin, cefatrizine, cefazaflur, cefazedone, cefazolin, cefbuperazone, cefcapene, cefdaloxime, cefepime, cefminox, cefoxitin, cefprozil, cefroxadine, ceftezole, cefuroxime, cefixime, cefdinir, cefditoren, cefepime, cefetamet, cefmenoxime, cefodizime, cefonicid, cefoperazone, ceforanide, cefotaxime, cefotiam, cefozopran, cephalexin, cefpimizole, cefpiramide, cefpirome, cefpodoxime, cefprozil, cefquinome, cefsulodin, ceftazidime, cefteram, ceftibuten, ceftiolene, ceftizoxime, ceftobiprole, ceftriaxone, cefuroxime, cefuzonam, cephamycin (cefoxitin, cefotetan, cefmetazole), oxacephem (flomoxef, latamoxef);
f). glycopeptides: bleomycin, vancomycin (oritavancin, telavancin), teicoplanin (dalbavancin), ramoplanin;
g). glycylcyclines: tigecycline;
h). β-lactamase inhibitors: penam (sulbactam, tazobactam), clavam (clavulanic acid);
i). lincosamides: clindamycin, lincomycin;
j). lipopeptides: daptomycin, A54145, calcium-dependent antibiotics (CDA);
k). macrolides: azithromycin, cethromycin, clarithromycin, dirithromycin, erythromycin, flurithromycin, josamycin, ketolide (telithromycin, cethromycin), midecamycin, miocamycin, oleandomycin, rifamycins (rifampicin, rifampin, rifabutin, rifapentine), rokitamycin, roxithromycin, spectinomycin, spiramycin, tacrolimus (FK506), troleandomycin, telithromycin;
l). monobactams: aztreonam, tigemonam;
m). oxazolidinones: linezolid;
n). penicillins: amoxicillin, ampicillin, pivampicillin, hetacillin, bacampicillin, metampicillin, talampicillin, azidocillin, azlocillin, benzylpenicillin, benzathine benzylpenicillin, benzathine phenoxymethylpenicillin, clometocillin, procaine benzylpenicillin, carbenicillin (carindacillin), cloxacillin, dicloxacillin, epicillin, flucloxacillin, mecillinam (pivmecillinam), mezlocillin, meticillin, nafcillin, oxacillin, penamecillin, penicillin, pheneticillin, phenoxymethylpenicillin, piperacillin, propicillin, sulbenicillin, temocillin, ticarcillin;
o). polypeptides: bacitracin, colistin, polymyxin B;
p). quinolones: alatrofloxacin, balofloxacin, ciprofloxacin, clinafloxacin, danofloxacin, difloxacin, enoxacin, enrofloxacin, floxin, garenoxacin, gatifloxacin, gemifloxacin, grepafloxacin, kano trovafloxacin, levofloxacin, lomefloxacin, marbofloxacin, moxifloxacin, nadifloxacin, norfloxacin, orbifloxacin, ofloxacin, pefloxacin, trovafloxacin, grepafloxacin, sitafloxacin, sparfloxacin, temafloxacin, tosufloxacin, trovafloxacin;
q). streptogramins: pristinamycin, quinupristin/dalfopristin;
r). sulfonamides: mafenide, prontosil, sulfacetamide, sulfamethizole, sulfanilimide, sulfasalazine, sulfisoxazole, trimethoprim, trimethoprim-sulfamethoxazole (co-trimoxazole);
s). steroid antibacterials: fusidic acid;
t). tetracyclines: doxycycline, chlortetracycline, clomocycline, demeclocycline, lymecycline, meclocycline, metacycline, minocycline, oxytetracycline, penimepicycline, rolitetracycline, tetracycline, glycylcyclines (including tigecycline);
u). antibiotics: annonacin, arsphenamine, bactoprenol inhibitors (Bacitracin), DADAL/AR inhibitors (cycloserine), dictyostatin, discodermolide, eleutherobin, epothilone, ethambutol, etoposide, faropenem, fusidic acid, furazolidone, isoniazid, laulimalide, metronidazole, mupirocin, mycolactone, NAM synthesis inhibitors (fosfomycin), nitrofurantoin, paclitaxel, platensimycin, pyrazinamide, quinupristin/dalfopristin, rifampicin (rifampin), tazobactam tinidazole, uvaricin;
(4). anti-viral drugs comprising:
a). entry/fusion inhibitors: aplaviroc, maraviroc, vicriviroc, gp41 (enfuvirtide), PRO 140, CD4 (ibalizumab);
b). integrase inhibitors: raltegravir, elvitegravir, globoidnan A;
c). maturation inhibitors: bevirimat, vivecon;
d). neuraminidase inhibitors: oseltamivir, zanamivir, peramivir;
e). nucleosides & nucleotides: abacavir, aciclovir, adefovir, amdoxovir, apricitabine, brivudine, cidofovir, clevudine, dexelvucitabine, didanosine (ddI), elvucitabine, emtricitabine (FTC), entecavir, famciclovir, fluorouracil (5-FU), 3′-fluoro-substituted 2′, 3′-dideoxynucleoside analogues (including the group consisting of 3′-fluoro-2′,3′-dideoxythymidine (FLT) and 3′-fluoro-2′,3′-dideoxyguanosine (FLG), fomivirsen, ganciclovir, idoxuridine, lamivudine (3TC), 1-nucleosides (including the group consisting of β-1-thymidine and β-1-2′-deoxycytidine), penciclovir, racivir, ribavirin, stampidine, stavudine (d4T), taribavirin (viramidine), telbivudine, tenofovir, trifluridine valaciclovir, valganciclovir, zalcitabine (ddC), zidovudine (AZT);
f). non-nucleosides: amantadine, ateviridine, capravirine, diarylpyrimidines (etravirine, rilpivirine), delavirdine, docosanol, emivirine, efavirenz, foscarnet (phosphonoformic acid), imiquimod, interferon alfa, loviride, lodenosine, methisazone, nevirapine, NOV-205, peginterferon alfa, podophyllotoxin, rifampicin, rimantadine, resiquimod (R-848), tromantadine;
g). protease inhibitors: amprenavir, atazanavir, boceprevir, darunavir, fosamprenavir, indinavir, lopinavir, nelfinavir, pleconaril, ritonavir, saquinavir, telaprevir (VX-950), tipranavir;
h). anti-virus drugs: abzyme, arbidol, calanolide a, ceragenin, cyanovirin-n, diarylpyrimidines, epigallocatechin gallate (EGCG), foscarnet, griffithsin, taribavirin (viramidine), hydroxyurea, KP-1461, miltefosine, pleconaril, portmanteau inhibitors, ribavirin, seliciclib;
(5). a-radioisotope selected from the group consisting of (radionuclides)³H, ¹¹C, ¹⁴C, ¹⁸F, ³²P, ³⁵S, ⁶⁴Cu, ⁶⁸Ga, ⁸⁶Y, ⁹⁹Tc, ¹¹¹In, ¹²³I, ¹²⁴I, ¹²⁵I, ¹³¹I, ¹³³Xe, ¹⁷⁷Lu, ²¹¹At, and ²¹³Bi;
(6). a chromophore molecule, which is capable of absorbing UV light, florescent light, IR light, near IR light, or visual light; a class or subclass of xanthophores, erythrophores, iridophores, leucophores, melanophores, cyanophores, fluorophore molecules which are fluorescent chemical compounds reemitting light upon light, visual phototransduction molecules, photophore molecules, luminescence molecules, luciferin compounds; Non-protein organic fluorophores, selected from: Xanthene derivatives (comprising fluorescein, rhodamine, Oregon green, eosin, and Texas red); Cyanine derivatives: (comprising cyanine, indocarbocyanine, oxacarbocyanine, thiacarbocyanine, and merocyanine); Squaraine derivatives and ring-substituted squaraines, including Seta, SeTau, and Square dyes; Naphthalene derivatives (comprising dansyl and prodan derivatives); Coumarin derivatives; Oxadiazole derivatives (comprising pyridyloxazole, nitrobenzoxadiazole and benzoxadiazole); Anthracene derivatives (comprising anthraquinones, including DRAQ5, DRAQ7 and CyTRAK Orange); Pyrene derivatives (cascade blue); Oxazine derivatives (comprising Nile red, Nile blue, cresyl violet, oxazine 170); Acridine derivatives (comprising proflavin, acridine orange, acridine yellow) Arylmethine derivatives (comprising auramine, crystal violet, malachite green); Tetrapyrrole derivatives (comprising porphin, phthalocyanine, bilirubin); analogs and derivatives of fluorophore compounds comprising CF dye, DRAQ and CyTRAK probes, BODIPY, Alexa Fluor, DyLight Fluor, Atto and Tracy, FluoProbes, Abberior Dyes, DY and MegaStokes Dyes, Sulfo Cy dyes, HiLyte Fluor, Seta, SeTau and Square Dyes, Quasar and Cal Fluor dyes, SureLight Dyes (APC, RPEPerCP, Phycobilisomes), APC, APCXL, RPE, BPE, Allophycocyanin (APC), Aminocoumarin, APC-Cy7 conjugates, BODIPY-FL, Cascade Blue, Cy2, Cy3, Cy3.5, Cy3B, Cy5, Cy5.5, Cy7, Fluorescein, FluorX, Hydroxycoumarin, Lissamine Rhodamine B, Lucifer yellow, Methoxycoumarin, NBD, Pacific Blue, Pacific Orange, PE-Cy5 conjugates, PE-Cy7 conjugates, PerCP, R-Phycoerythrin(PE), Red 613, Seta-555-Azide, Seta-555-DBCO, Seta-555-NHS, Seta-580-NHS, Seta-680-NHS, Seta-780-NHS, Seta-APC-780, Seta-PerCP-680, Seta-R-PE-670, SeTau-380-NHS, SeTau-405-Maleimide, SeTau-405-NHS, SeTau-425-NHS, SeTau-647-NHS, Texas Red, TRITC, TruRed, X-Rhodamine, 7-AAD (7-aminoactinomycin D, CG-selective), Acridine Orange, Chromomycin A3, CyTRAK Orange (red excitation dark), DAPI, DRAQ5, DRAQ7, Ethidium Bromide, Hoechst33258, Hoechst33342, LDS 751, Mithramycin, Propidiumlodide (PI), SYTOX Blue, SYTOX Green, SYTOX Orange, Thiazole Orange, TO-PRO: Cyanine Monomer, TOTO-1, TO-PRO-1, TOTO-3, TO-PRO-3, YOSeta-1, YOYO-1; a fluorophore compound: comprising DCFH (2′7′-Dichorodihydro-fluorescein, oxidized form), DHR (Dihydrorhodamine 123, oxidized form, light catalyzes oxidation), Fluo-3 (AM ester, pH>6), Fluo-4 (AM ester, pH 7.2), Indo-1 (AM ester, low/high calcium (Ca2+)), SNARF(pH 6/9), Allophycocyanin(APC), AmCyan1 (tetramer, Clontech), AsRed2 (tetramer, Clontech), Azami Green (monomer), Azurite, B-phycoerythrin (BPE), Cerulean, CyPet, DsRed monomer (Clontech), DsRed2 (“RFP”), EBFP, EBFP2, ECFP, EGFP (weak dimer), Emerald (weak dimer), EYFP (weak dimer), GFP (S65A mutation), GFP (S65C mutation), GFP (S65L mutation), GFP (S65T mutation), GFP (Y66F mutation), GFP (Y66H mutation), GFP (Y66W mutation), GFPuv, HcRed1, J-Red, Katusha, Kusabira Orange (monomer, MBL), mCFP, mCherry, mCitrine, Midoriishi Cyan (dimer, MBL), mKate (TagFP635, monomer), mKeima-Red (monomer), mKO, mOrange, mPlum, mRaspberry, mRFP1 (monomer), mStrawberry, mTFP1, mTurquoise2, P3 (phycobilisome complex), Peridinin Chlorophyll (PerCP), R-phycoerythrin (RPE), T-Sapphire, TagCFP (dimer), TagGFP (dimer), TagRFP (dimer), TagYFP (dimer), tdTomato (tandem dimer), Topaz, TurboFP602 (dimer), TurboFP635 (dimer), TurboGFP (dimer), TurboRFP (dimer), TurboYFP (dimer), Venus, Wild Type GFP, YPet, ZsGreen1 (tetramer), ZsYellow1 (tetramer) and their derivatives;
(7). cell-binding ligands or receptor agonists: Folate derivatives; Glutamic acid urea derivatives; Somatostatin and its analogs (selected from the group consisting of octreotide (Sandostatin) and lanreotide (Somatuline)); Aromatic sulfonamides; Pituitary adenylate cyclase activating peptides (PACAP) (PAC1); Vasoactive intestinal peptides (VIP/PACAP) (VPAC1, VPAC2); Melanocyte-stimulating hormones (α-MSH); Cholecystokinins (CCK)/gastrin receptor agonists; Bombesins (selected from the group consisting of Pyr-Gln-Arg-Leu-Gly-Asn-Gln-Trp-Ala-Val-Gly-His-Leu-Met-NH₂)/gastrin-releasing peptide (GRP); Neurotensin receptor ligands (NTR1, NTR2, NTR3); Substance P (NK1 receptor) ligands; Neuropeptide Y (Y1-Y6); Homing Peptides include RGD (Arg-Gly-Asp), NGR (Asn-Gly-Arg), dimeric and multimeric cyclic RGD peptides (selected from cRGDfV), TAASGVRSMH and LTLRWVGLMS (Chondroitin sulfate proteoglycan NG2 receptor ligands) and F3 peptides; Cell Penetrating Peptides (CPPs); Peptide Hormones, selected from the group consisting of luteinizing hormone-releasing hormone (LHRH) agonists and antagonists, and gonadotropin-releasing hormone (GnRH) agonist, acts by targeting follicle stimulating hormone (FSH) and luteinising hormone (LH), as well as testosterone production, selected from the group consisting of buserelin (Pyr-His-Trp-Ser-Tyr-D-Ser(OtBu)-Leu-Arg-Pro-NHEt), Gonadorelin (Pyr-His-Trp-Ser-Tyr-Gly-Leu-Arg-Pro-Gly-NH₂), Goserelin (Pyr-His-Trp-Ser-Tyr-D-Ser(OtBu)-Leu-Arg-Pro-AzGly-NH₂), Histrelin (Pyr-His-Trp-Ser-Tyr-D-His(N-benzyl)-Leu-Arg-Pro-NHEt), leuprolide (Pyr-His-Trp-Ser-Tyr-D-Leu-Leu-Arg-Pro-NHEt), Nafarelin (Pyr-His-Trp-Ser-Tyr-2Nal-Leu-Arg-Pro-Gly-NH₂), Triptorelin (Pyr-His-Trp-Ser-Tyr-D-Trp-Leu-Arg-Pro-Gly-NH₂), Nafarelin, Deslorelin, Abarelix (Ac-D-2Nal-D-4-chloroPhe-D-3-(3-pyridyl)Ala-Ser-(N-Me)Tyr-D-Asn-Leu-isopropylLys-Pro-DAla-NH₂), Cetrorelix (Ac-D-2Nal-D-4-chloroPhe-D-3-(3-pyridyl)Ala-Ser-Tyr-D-Cit-Leu-Arg-Pro-D-Ala-NH₂), Degarelix (Ac-D-2Nal-D-4-chloroPhe-D-3-(3-pyridyl)Ala-Ser-4-aminoPhe(L-hydroorotyl)-D-4-aminoPhe(carba-moyl)-Leu-isopropylLys-Pro-D-Ala-NH₂), and Ganirelix (Ac-D-2Nal-D-4-chloroPhe-D-3-(3-pyridyl)Ala-Ser-Tyr-D-(N9, N10-diethyl)-homoArg-Leu-(N9, N10-diethyl)-homoArg-Pro-D-Ala-NH₂); Pattern Recognition Receptor (PRRs), selected from the group consisting of Toll-like receptors' (TLRs) ligands, C-type lectins and Nodlike Receptors' (NLRs) ligands; Calcitonin receptor agonists; integrin receptors' and their receptor subtypes' (selected from the group consisting of α_{V}β₁, α_{V}β₃, α_{V}β₅, α_{V}β₆, α₆β₄, α₇β₂, α_{L}β₂, α_{IIb}β₃) agonists (selected from the group consisting of GRGDSPK, cyclo(RGDfV) (L1) and its derives [cyclo(-N(Me)R-GDfV), cyclo(R-Sar-DfV), cyclo(RG-N(Me)D-fV), cyclo(RGD-N(Me)f-V), cyclo(RGDf-N(Me)V-)(Cilengitide)]; Nanobody (a derivative of VHH (camelid Ig)); Domain antibodies (dAb, a derivative of VH or VL domain); Bispecific T cell Engager (BiTE, a bispecific diabody); Dual Affinity ReTargeting (DART, a bispecific diabody); Tetravalent tandem antibodies (TandAb, a dimerized bispecific diabody); Anticalin (a derivative of Lipocalins); Adnectins (10th FN3 (Fibronectin)); Designed Ankyrin Repeat Proteins (DARPins); Avimers; EGF receptors and VEGF receptors' agonists
(8). pharmaceutically acceptable salts, acids, derivatives, hydrate or hydrated salt; or a crystalline structure; or an optical isomer, racemate, diastereomer or enantiomer of any of the above drugs.

11. The conjugate compound according to claim 1, wherein the cytotoxic molecule is a chromophore molecule.

12. The conjugate compound according to claim 1, wherein the cytotoxic molecule is a polyalkylene glycol comprising poly(ethylene glycol) (PEGs), poly(propylene glycol), a copolymer of ethylene oxide or propylene oxide, or an analog thereof.

13. The conjugate compound according to claim 1, wherein the cytotoxic molecule is a cell-binding ligand, a cell receptor agonist, or a cell receptor binding molecule.

14. The conjugate compound of claim 1, wherein the cytotoxic molecule is selected from the group consisting of tubulysins, calicheamicins, auristatins, maytansinoids, CC-1065 analogs, daunorubicin and doxorubicin compounds, taxanoids (taxanes), cryptophycins, epothilones, benzodiazepine dimers (comprising pyrrolobenzodiazepine dimers (PBD), tomaymycin dimers, anthramycin dimers, indolinobenzodiazepine dimers, imidazobenzothiadiazepine dimers, or oxazolidinobenzodiazepine dimers and their derivatives), calicheamicins and the enediyne antibiotics, actinomycins, amatoxins, amanitins, azaserines, bleomycins, epirubicins, tamoxifen, idarubicin, dolastatins/auristatins (comprising monomethyl auristatin E, MMAE, MMAF, auristatin PYE, auristatin TP, Auristatins 2-AQ, 6-AQ, EB (AEB), EFP (AEFP) and their analogs), duocarmycins, geldanamycins, methotrexates, thiotepa, vindesines, vincristines, hemiasterlins, nazumamides, microginins, radiosumins, alterobactins, microsclerodermins, theonellamides, esperamicins, siRNA, miRNA, piRNA, nucleolytic enzymes, and/or pharmaceutically acceptable salts, acids, or/and their analogues, derivatives, hydrate or hydrated salt; or a crystalline structure; or an optical isomer, racemate, diastereomer or enantiomer of any of the above drugs.

15. The conjugate compound according to claim 1, wherein the cell binding molecule is selected from the group consisting of an antibody, a protein, probody, nanobody, a vitamin (including folate), peptides, a polymeric micelle, a liposome, a lipoprotein-based drug carrier, a nano-particle drug carrier, a dendrimer, and a molecule or a particle of said above coating with cell-binding ligands, or a combination of said above.

16. The conjugate compound according to claim 1, wherein the cell binding molecule is selected from an antibody, an anti-body-like protein, a full-length antibody (polyclonal antibody, monoclonal antibody, antibody dimer, antibody multimer), or multispecific antibody (selected from, bispecific antibody, trispecific antibody, or tetraspecific antibody); a single chain antibody, an antibody fragment that binds to the target cell, a monoclonal antibody, a single chain monoclonal antibody, or a monoclonal antibody fragment that binds the target cell, a chimeric antibody, a chimeric antibody fragment that binds to the target cell, a domain antibody, a domain antibody fragment that binds to the target cell, a resurfaced antibody, a resurfaced single chain antibody, or a resurfaced antibody fragment that binds to the target cell, a humanized antibody or a resurfaced antibody, a humanized single chain antibody, or a humanized antibody fragment that binds to the target cell, anti-idiotypic (anti-Id) antibodies, CDR's, diabody, triabody, tetrabody, miniantibody, a probody, a probody fragment, small immune proteins (SIP), a lymphokine, a hormone, a vitamin, a growth factor, a colony stimulating factor, a nutrient-transport molecule, large molecular weight proteins, nanoparticles or polymers modified with antibodies or large molecular weight proteins.

17. The conjugate compound according to claim 1, wherein the cell binding molecule is capable of targeting against a tumor cell, a virus infected cell, a microorganism infected cell, a parasite infected cell, an autoimmune disease cell, an activated tumor cells, a myeloid cell, an activated T-cell, an affecting B cell, or a melanocyte, or any cells expressing any one of the following antigens or receptors: CD2, CD2R, CD3, CD3gd, CD3e, CD4, CD5, CD6, CD7, CD8, CD8a, CD8b, CD9, CD10, CD11a, CD11b, CD11c, CD12, CD12w, CD13, CD14, CD15, CD15s, CD15u, CD16, CD16a, CD16b, CD17, CDw17, CD18, CD19, CD20, CD21, CD22, CD23, CD24, CD25, CD26, CD27, CD28, CD29, CD30, CD31, CD32, CD33, CD34, CD35, CD36, CD37, CD38, CD39, CD40, CD41, CD42, CD42a, CD42b, CD42c, CD42d, CD43, CD44, CD44R, CD45, CD45RA, CD45RB, CD45RO, CD46, CD47, CD47R, CD48, CD49a, CD49b, CD49c, CD49e, CD49f, CD50, CD51, CD52, CD53, CD54, CD55, CD56, CD57, CD58, CD59, CD60, CD60a, CD60b, CD60c, CD61, CD62E, CD62L, CD62P, CD63, CD64, CD65, CD65s, CD66, CD66a, CD66b, CD66c, CD66d, CD66e, CD66f, CD67, CD68, CD69, CD70, CD71, CD72, CD73, CD74, CD74, CD75, CD75s, CD76, CD77, CD78, CD79, CD79a, CD79b, CD80, CD81, CD82, CD83, CD84, CDw84, CD85, CD86, CD87, CD88, CD89, CD90, CD91, CD92, CDw92, CD93, CD94, CD95, CD96, CD97, CD98, CD99, CD99R, CD100, CD101, CD102, CD103, CD104, CD105, CD106, CD107, CD107a, CD107b, CD108, CD109, CD110, CD111, CD112, CD113, CDw113, CD114, CD115, CD116, CD117, CD118, CD119, CDw119, CD120a, CD120b, CD121a, CD121b, CDw121b, CD122, CD123, CDw123, CD124, CD125, CDw125, CD126, CD127, CD128, CDw128, CD129, CD130, CD131, CDw131, CD132, CD133, CD134, CD135, CD136, CDw136, CD137, CDw137, CD138, CD139, CD140a, CD140b, CD141, CD142, CD143, CD144, CD145, CDw145, CD146, CD147, CD148, CD149, CD150, CD151, CD152, CD153, CD154, CD155, CD156a, CD156b, CDw156c, CD157, CD158a, CD158b, CD159a, CD159b, CD159c, CD160, CD161, CD162, CD162R, CD163, CD164, CD165, CD166, CD167, CD167a, CD168, CD169, CD170, CD171, CD172a, CD172b, CD172g, CD173, CD174, CD175, CD175s, CD176, CD177, CD178, CD179, CD180, CD181, CD182, CD183, CD184, CD185, CD186, CDw186, CD187, CD188, CD189, CD190, Cd191, CD192, CD193, CD194, CD195, CD196, CD197, CD198, CDw198, CD199, CDw199, CD200, CD200a, CD200b, CD201, CD202, CD202b, CD203, CD203c, CD204, CD205, CD206, CD207, CD208, CD209, CD210, CDw210, CD212, CD213a1, CD213a2, CDw217, CDw218a, CDw218b, CD220, CD221, CD222, CD223, CD224, CD225, CD226, CD227, CD228, CD229, CD230, CD231, CD232, CD233, CD234, CD235a, CD235ab, CD235b, CD236, CD236R, CD238, CD239, CD240, CD240CE, CD240D, CD241, CD242, CD243, CD244, CD245, CD246, CD247, CD248, CD249, CD252, CD253, CD254, CD256, CD257, CD258, CD261, CD262, CD263, CD265, CD266, CD267, CD268, CD269, CD271, CD273, CD274, CD275, CD276 (B7-H3), CD277, CD278, CD279, CD280, CD281, CD282, CD283, CD284, CD289, CD292, CDw293, CD294, CD295, CD296, CD297, CD298, CD299, CD300a, CD300c, CD300e, CD301, CD302, CD303, CD304, CD305, CD306, CD309, CD312, CD314, CD315, CD316, CD317, CD318, CD319, CD320, CD321, CD322, CD324, CDw325, CD326, CDw327, CDw328, CDw329, CD331, CD332, CD333, CD334, CD335, CD336, CD337, CDw338, CD339, 4-1BB, 5AC, 5T4 (Trophoblast glycoprotein, TPBG, 5T4, Wnt-Activated Inhibitory Factor 1 or WAIF 1), Adenocarcinoma antigen, AGS-5, AGS-22M6, Activin receptor-like kinase 1, AFP, AKAP-4, ALK, Alpha intergrin, Alpha v beta6, Amino-peptidase N, Amyloid beta, Androgen receptor, Angiopoietin 2, Angiopoietin 3, Annexin A1, Anthrax toxin protective antigen, Anti-transferrin receptor, AOC3 (VAP-1), B7-H3, Bacillus anthracis anthrax, BAFF (B-cell activating factor), BCMA, B-lymphoma cell, bcr-abl, Bombesin, BORIS, C5, C242 antigen, CA125 (carbohydrate antigen 125, MUC16), CA-IX (or CAIX, carbonic anhydrase 9), CALLA, CanAg, Canis lupus familiaris IL31, Carbonic anhydrase IX, Cardiac myosin, CCL11 (C—C motif chemokine 11), CCR₄ (C—C chemokine receptor type 4), CCR₅, CD3E (epsilon), CEA (Carcinoembryonic antigen), CEACAM3, CEACAM5 (carcinoembryonic antigen), CFD (Factor D), Ch4D5, Cholecystokinin 2 (CCK2R), CLDN18 (Claudin-18), Clumping factor A, cMet, CRIPTO, FCSF1R (Colony stimulating factor 1 receptor), CSF2 (colony stimulating factor 2, Granulocyte-macrophage colony-stimulating factor (GM-CSF)), CSP4, CTLA4 (cytotoxic T-lymphocyte-associated protein 4), CTAA16.88 tumor antigen, CXCR4, C—X—C chemokine receptor type 4, cyclic ADP ribose hydrolase, Cyclin B1, CYP1B1, Cytomegalovirus, Cytomegalovirus glycoprotein B, Dabigatran, DLL3 (delta-like-ligand 3), DLL4 (delta-like-ligand 4), DPP4 (Dipeptidyl-peptidase 4), DR5 (Death receptor 5), E. coli shiga toxin type-1, E. coli shiga toxin type-2, ED-B, EGFL7 (EGF-like domain-containing protein 7), EGFR, EGFRII, EGFRvIII, Endoglin, Endothelin B receptor, Endotoxin, EpCAM (epithelial cell adhesion molecule), EphA2, Episialin, ERBB2 (Epidermal Growth Factor Receptor 2), ERBB3, ERG (TMPRSS2 ETS fusion gene), Escherichia coli, ETV6-AML, FAP (Fibroblast activation protein alpha), FCGR1, alpha-Fetoprotein, Fibrin II, beta chain, Fibronectin extra domain-B, FOLR (folate receptor), Folate receptor alpha, Folate hydrolase, Fos-related antigen 1F protein of respiratory syncytial virus, Frizzled receptor, Fucosyl GM1, GD2 ganglioside, G-28 (a cell surface antigen glyvolipid), GD3 idiotype, GloboH, Glypican 3, N-glycolylneuraminic acid, GM3, GMCSF receptor a-chain, Growth differentiation factor 8, GP100, GPNMB (Trans-membrane glycoprotein NMB), GUCY2C (Guanylate cyclase 2C, guanylyl cyclase C(GC-C), intestinal Guanylate cyclase, Guanylate cyclase-C receptor, Heat-stable enterotoxin receptor (hSTAR)), Heat shock proteins, Hemagglutinin, Hepatitis B surface antigen, Hepatitis B virus, HER1 (human epidermal growth factor receptor 1), HER2, HER2/neu, HER3 (ERBB-3), IgG4, HGF/SF (Hepatocyte growth factor/scatter factor), HHGFR, HIV-1, Histone complex, HLA-DR (human leukocyte antigen), HLA-DR10, HLA-DRB, HMWMAA, Human chorionic gonadotropin, HNGF, Human scatter factor receptor kinase, HPV E6/E7, Hsp90, hTERT, ICAM-1 (Intercellular Adhesion Molecule 1), Idiotype, IGF1R (IGF-1, insulin-like growth factor 1 receptor), IGHE, IFN-γ, Influenza hemagglutinin, IgE, IgE Fc region, IGHE, interleukins (comprising IL-1, IL-2, IL-3, IL-4, IL-5, IL-6, IL-6R, IL-7, IL-8, IL-9, IL-10, IL-11, IL-12, IL-13, IL-15, IL-17, IL-17A, IL-18, IL-19, IL-20, IL-21, IL-22, IL-23, IL-27, or IL-28), IL31RA, ILGF2 (Insulin-like growth factor 2), Integrins (α4, α_{IIb}β3, αvβ3, α₄β₇, α5β1, α6β4, α7β7, αllβ3, α5β5, αvβ5), Interferon gamma-induced protein, ITGA2, ITGB2, KIR2D, Kappa Ig, LCK, Le, Legumain, Lewis-Y antigen, LFA-1 (Lymphocyte function-associated antigen 1, CD11a), LHRH, LINGO-1, Lipoteichoic acid, LIV1A, LMP2, LTA, MAD-CT-1, MAD-CT-2, MAGE-1, MAGE-2, MAGE-3, MAGE A1, MAGE A3, MAGE 4, MART1, MCP-1, MIF (Macrophage migration inhibitory factor, or glycosylation-inhibiting factor (GIF)), MS4A1 (membrane-spanning 4-domains subfamily A member 1), MSLN (mesothelin), MUC1 (Mucin 1, cell surface associated (MUC1) or polymorphic epithelial mucin (PEM)), MUC1-KLH, MUC16 (CA125), MCP1 (monocyte chemotactic protein 1), MelanA/MART1, ML-IAP, MPG, MS4A1 (membrane-spanning 4-domains subfamily A), MYCN, Myelin-associated glycoprotein, Myostatin, NA17, NARP-1, NCA-90 (granulocyte antigen), Nectin-4 (ASG-22ME), NGF, Neural apoptosis-regulated proteinase 1, NOGO-A, Notch receptor, Nucleolin, Neu oncogene product, NY-BR-1, NY-ESO-1, OX-40, OxLDL (Oxidized low-density lipoprotein), OY-TES1, P21, p53 nonmutant, P97, Page4, PAP, Paratope of anti-(N-glycolylneuraminic acid), PAX3, PAX5, PCSK9, PDCD1 (PD-1, Programmed cell death protein 1), PDGF-Rα (Alpha-type platelet-derived growth factor receptor), PDGFR-13, PDL-1, PLAC1, PLAP-like testicular alkaline phosphatase, Platelet-derived growth factor receptor beta, Phosphate-sodium co-transporter, PMEL 17, Polysialic acid, Proteinase3 (PR1), Prostatic carcinoma, PS (Phosphatidylserine), Prostatic carcinoma cells, Pseudomonas aeruginosa, PSMA, PSA, PSCA, Rabies virus glycoprotein, RHD (Rh polypeptide 1 (RhPI)), Rhesus factor, RANKL, RhoC, Ras mutant, RGS5, ROBO4, Respiratory syncytial virus, RON, ROR1, Sarcoma translocation breakpoints, SART3, Sclerostin, SLAMF7 (SLAM family member 7), Selectin P, SDC1 (Syndecan 1), sLe(a), Somatomedin C, SIP (Sphingosine-1-phosphate), Somatostatin, Sperm protein 17, SSX2, STEAP1 (six-transmembrane epithelial antigen of the prostate 1), STEAP2, STn, TAG-72 (tumor associated glycoprotein 72), Survivin, T-cell receptor, T cell transmembrane protein, TEM1 (Tumor endothelial marker 1), TENB2, Tenascin C (TN-C), TGF-α, TGF-β (Transforming growth factor beta), TGF-β1, TGF-β2 (Transforming growth factor-beta 2), Tie (CD202b), Tie2, TIM-1 (CDX-014), Tn, TNF, TNF-α, TNFRSF8, TNFRSF10B (tumor necrosis factor receptor superfamily member 10B), TNFRSF-13B (tumor necrosis factor receptor superfamily member 13B), TPBG (trophoblast glycoprotein), TRAIL-R₁ (Tumor necrosis apoprosis Inducing ligand Receptor 1), TRAILR2 (Death receptor 5 (DR5)), tumor-associated calcium signal transducer 2, tumor specific glycosylation of MUC1, TWEAK receptor, TYRP1 (glycoprotein 75), TRP-2, Tyrosinase, VCAM-1, VEGF, VEGF-A, VEGF-2, VEGFR-1, VEGFR2, or vimentin, WT1, XAGE 1, or cells expressing any insulin growth factor receptors, or any epidermal growth factor receptors.

18. The conjugate compound according to claim 17, wherein the tumor cell is selected from the group consisting of lymphoma cells, myeloma cells, renal cells, breast cancer cells, prostate cancer cells, ovarian cancer cells, colorectal cancer cells, gastric cancer cells, squamous cancer cells, small-cell lung cancer cells, none small-cell lung cancer cells, testicular cancer cells, malignant cells, and cells that grow and divide at an unregulated, quickened pace to cause cancers.

19. The conjugate compound of claim 1, wherein the conjugate compound of Formula (I) is selected from the group consisting of structures of Ac01, Ac02, Ac03, Ac04, Ac05, Ac06, and Ac07 as following: 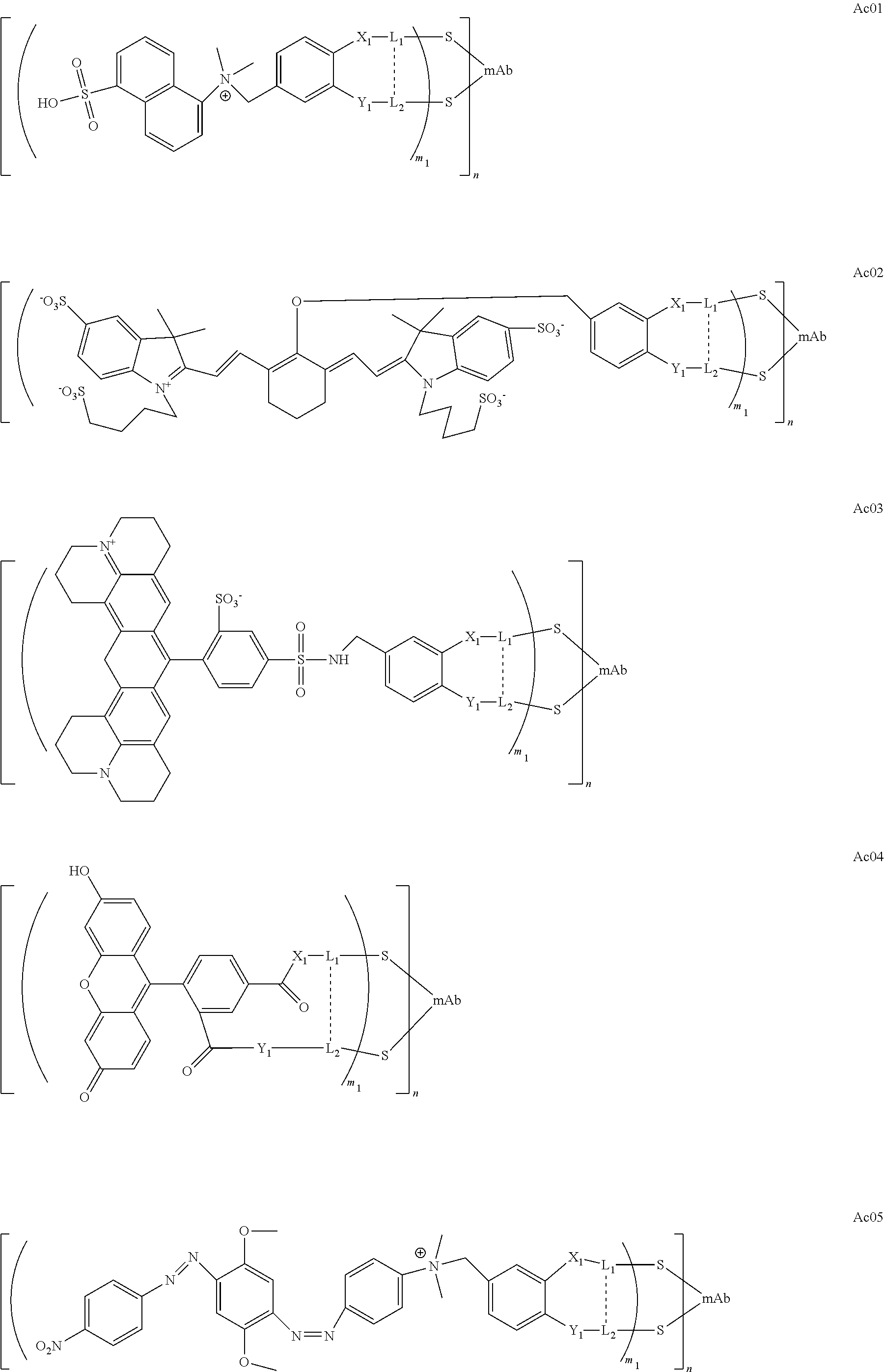 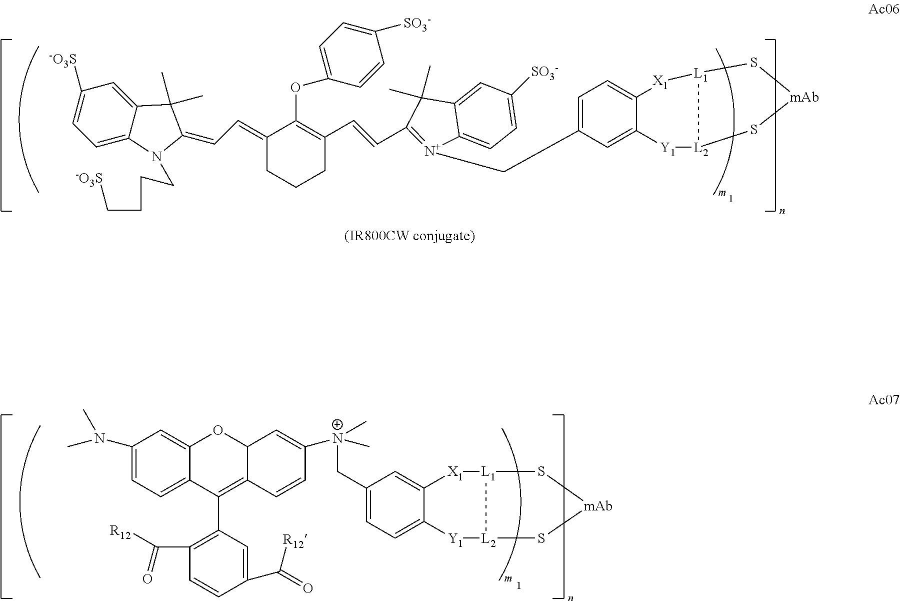
wherein “-----” is a single bond, a double bond, or absent; X₁, and Y₁ are independently O, NH, NHNH, NR₅, S, C(O)O, C(O)NH, OC(O)NH, OC(O)O, NHC(O)NH, NHC(O)S, OC(O)N(R₁), N(R₁)C(O)N(R₁), CH, C(O)NHNHC(O) or C(O)NR₁; mAb is antibody; n and m₁ are independently 1-20; R₁₂ and R₁₂′ are independently OH, NH₂, NHR₁, NHNH₂, NHNHCOOH, O—R₁—COOH, NH—R₁—COOH, NH-(Aa)ₙCOOH, O(CH₂CH₂O)ₚCH₂CH₂OH, O(CH₂CH₂O)ₚCH₂CH₂NH₂, NH(CH₂CH₂O)ₚCH₂CH₂NH₂, O(CH₂CH₂O)ₚCH₂CH₂COOH, NH(CH₂CH₂O)ₚCH₂CH₂COOH, O(CH₂CH₂O)ₚCH₂CH₂NHSO₃H, NH(CH₂CH₂O)ₚCH₂CH₂NHSO₃H, R₁—NHSO₃H, NH—R₁—NHSO₃H, O(CH₂CH₂O)ₚCH₂CH₂NHPO₃H₂, NH(CH₂CH₂O)ₚCH₂CH₂NHPO₃H₂, R₁—NHPO₃H₂, R₁—OPO₃H₂, O(CH₂CH₂O)ₚCH₂CH₂OPO₃H₂, NH(CH₂CH₂O)ₚCH₂CH₂NHPO₃H₂, OR₁—NHPO₃H₂, NH—R₁—NHPO₃H₂, NH—Ar—COOH, or NH—Ar—NH₂, wherein p=0-5000, Aa is aminoacids; R₁, L₁, and L₂ are defined the same as in claim 1.

20. The conjugate compound of claim 1, wherein the conjugate compound of Formula (I) is selected from structures of T01, T02, T03, T04, T05, T06, T07, T08, T09, T10, and T11 as following: 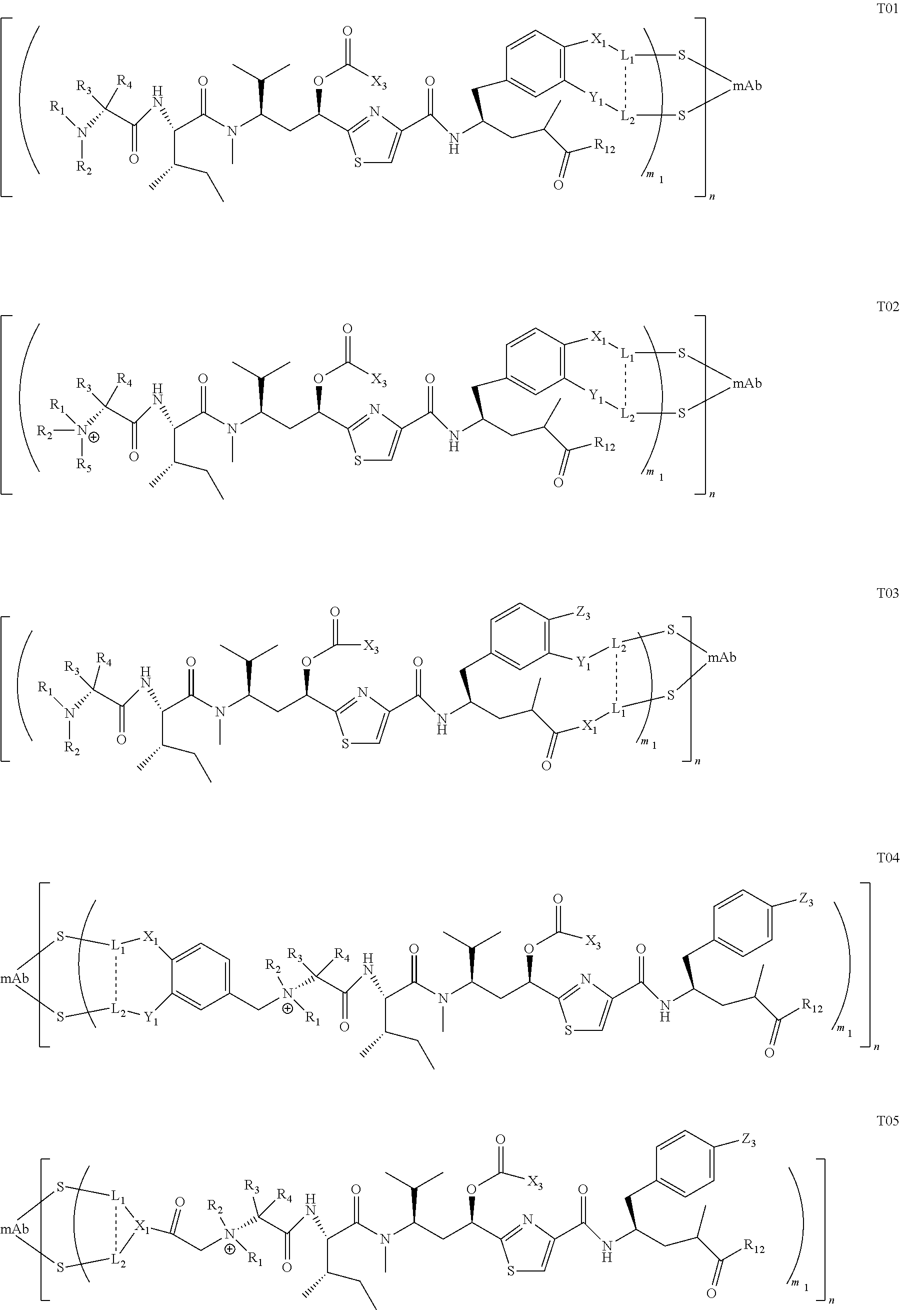 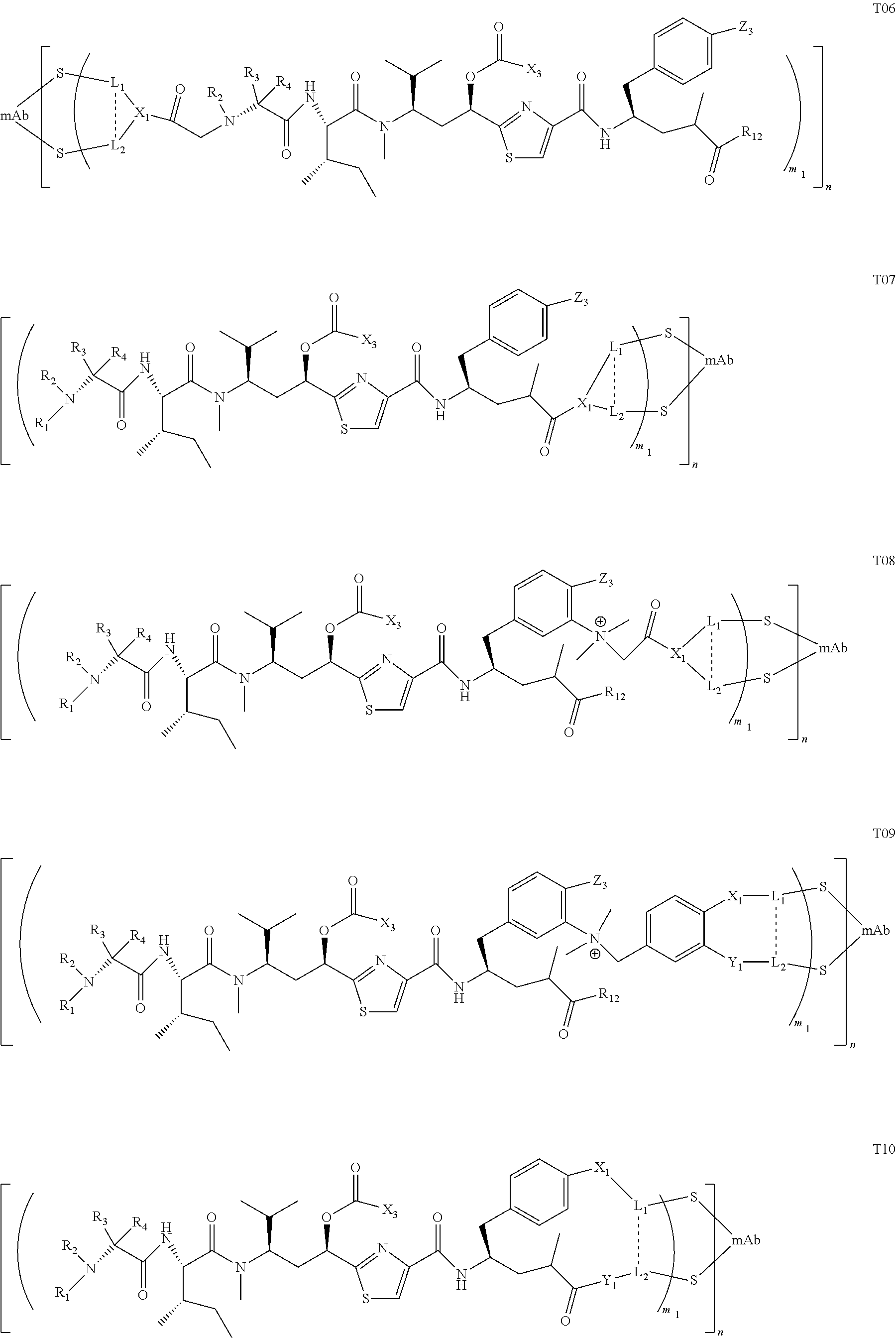 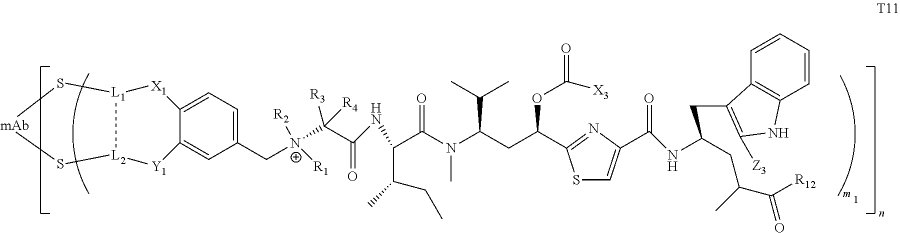
wherein “-----” is a single bond, a double bond, absent; X₁, and Y₁ are independently O, NH, NHNH, NR₅, S, C(O)O, C(O)NH, OC(O)NH, OC(O)O, NHC(O)NH, NHC(O)S, OC(O)N(R₁), N(R₁)C(O)N(R₁), CH, C(O)NHNHC(O), or C(O)NR₁; mAb is antibody; R₁₂ is OH, NH₂, NHR₁, NHNH₂, NHNHCOOH, O—R₁—COOH, NH—R₁—COOH, NH-(Aa)ₙCOOH, O(CH₂CH₂O)ₚCH₂CH₂OH, O(CH₂CH₂O)CH₂CH₂NH₂, NH(CH₂CH₂O)ₚCH₂—CH₂NH₂, NR₁R₁′, NHOH, NHOR₁, O(CH₂CH₂O)ₚCH₂CH₂COOH, NH(CH₂CH₂O)ₚCH₂CH₂₋COOH, NH—Ar—COOH, NH—Ar—NH₂, O(CH₂CH₂O)ₚCH₂CH₂NHSO₃H, NH(CH₂CH₂O)ₚCH₂CH₂NHSO₃H, R₁—NHSO₃H, NH—R₁—NHSO₃H, O(CH₂CH₂O)ₚCH₂CH₂NHPO₃H₂, NH(CH₂CH₂O)ₚCH₂CH₂NHPO₃H₂, OR₁, R₁—NHPO₃H₂, R₁—OPO₃H₂, O(CH₂CH₂O)ₚCH₂CH₂OPO₃H₂, OR₁—NHPO₃H₂, NH—R₁—NHPO₃H₂, NH(CH₂CH₂NH)ₚCH₂CH₂NH₂, NH(CH₂CH₂S)ₚCH₂CH₂NH₂, NH(CH₂CH₂NH)ₚCH₂CH₂OH, NH(CH₂CH₂S)ₚCH₂CH₂OH, NH—R₁—NH₂, or NH(CH₂CH₂O)ₚCH₂CH₂NHPO₃H₂, wherein Aa is 1-8 aminoacids; n and m₁ are independently 1-20; p is 1-5000; R₁, R₁′, R₂, R₃, and R₄ are independently H, C₁-C₈ linear or branched alkyl, amide, or amine; C₂-C₈ aryl, alkenyl, alkynyl, heteroaryl, heteroalkyl, alkylcycloalkyl, ester, ether, heterocycloalkyl, or acyloxylamine; or a peptide containing 1-8 aminoacids, or polyethyleneoxy unit having formula (OCH₂CH₂)ₚ or (OCH₂CH(CH₃))ₚ, wherein p is an integer from 1 to about 5000; or two Rs: R₁R₂, R₂R₃, R₁R₃ or R₃R₄ form 3˜8 member cyclic ring of alkyl, aryl, heteroaryl, heteroalkyl, or alkylcycloalkyl group; X₃ is H, CH₃, CH₂CH₃, C₃Ho, or X₁′R₁′, wherein X₁′ is NH, N(CH₃), NHNH, O, or S; R₁′ is H or C₁-C₈ linear or branched alkyl, aryl, heteroaryl, heteroalkyl, alkylcycloalkyl, or acyloxylamine; R_{3′} is H or C₁-C₆ linear or branched alkyl; Z₃ is H, COOR₁, NH₂, NHR₁, OR₁, CONHR₁, NHCOR₁, OCOR₁, OP(O)(OM₁)(OM₂), OCH₂OP(O)(OM₁)(OM₂), OSO₃M₁, R₁, O-glycoside (glucoside, galactoside, mannoside, glucuronoside/glucuronide, alloside, fructoside), NH-glycoside, S-glycoside or CH₂-glycoside; M₁ and M₂ are independently H, Na, K, Ca, Mg, NH₄, or NR₁R₂R₃; L₁, and L₂ are defined the same as in claim 1.

21. The conjugate compound of claim 1, wherein the conjugate compound of Formula (I) is selected from structures of C01 and C02 as following: 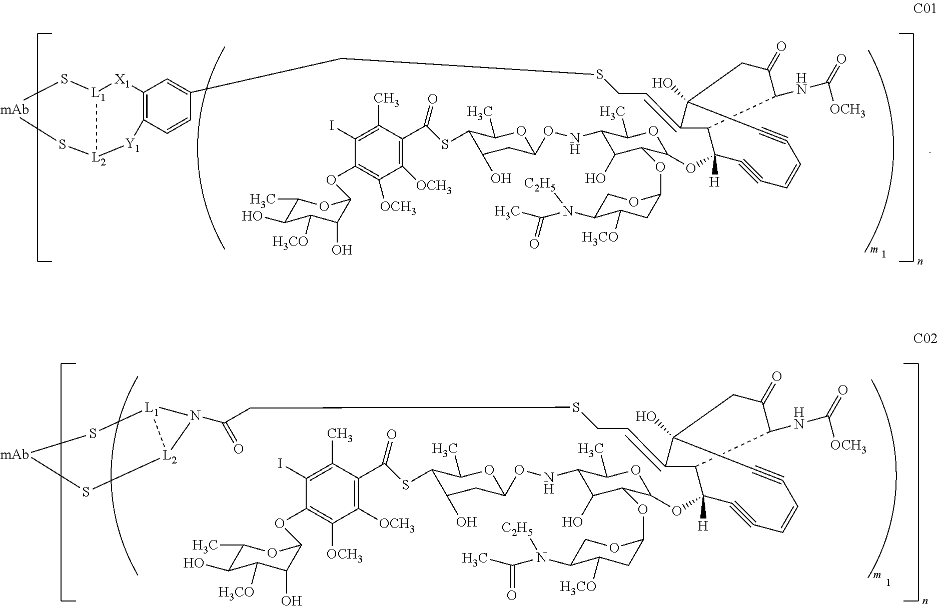
wherein “-----” is a single bond, a double bond, or absent; X₁ and Y₁ are independently O, NH, NHNH, NR₅, S, C(O)O, C(O)NH, OC(O)NH, OC(O)O, NHC(O)NH, NHC(O)S, OC(O)N(R₁), N(R₁)C(O)N(R₁), CH, C(O)NHNHC(O) or C(O)NR₁; mAb is antibody; n and m₁ are independently 1-20; p is 1-5000; R₁, L₁, and L₂ are defined the same as in claim 1.

22. The conjugate compound of claim 1, wherein the conjugate compound is selected from structures of following My01, My02, My03, My04, My05, and My06: 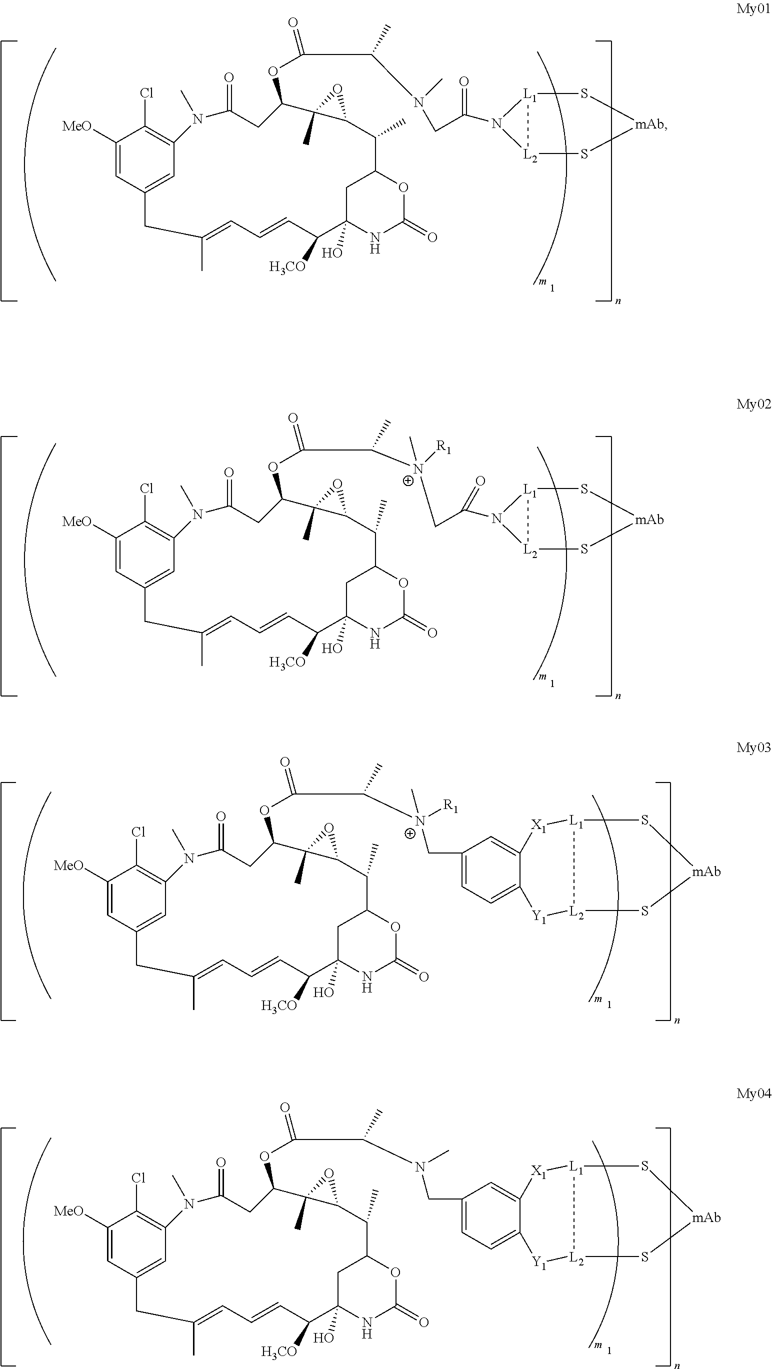 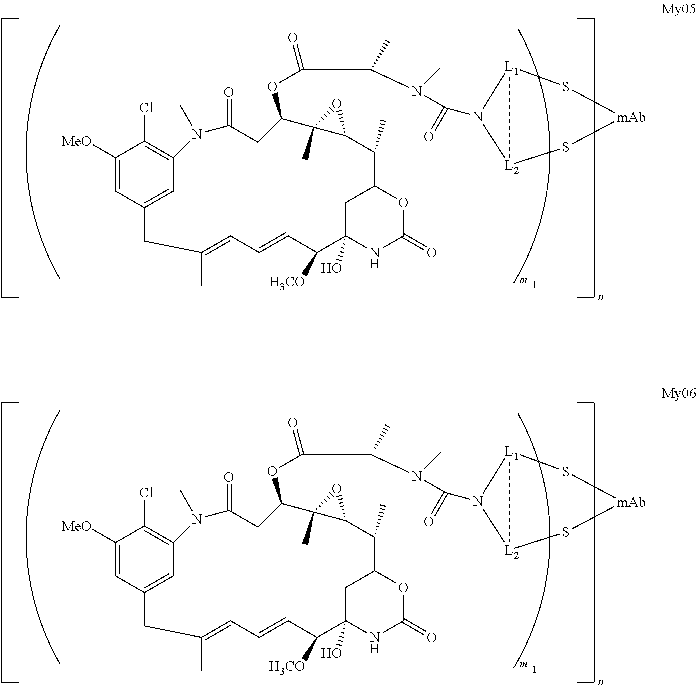
wherein “-----” is a single bond, a double bond, or absent; X₁, and Y₁ are independently O, NH, NHNH, NR₅, S, C(O)O, C(O)NH, OC(O)NH, OC(O)O, NHC(O)NH, NHC(O)S, OC(O)N(R₁), N(R₁)C(O)N(R₁), CH, C(O)NHNHC(O) or C(O)NR₁; mAb is antibody; n and m₁ are independently 1-20; p is 1-5000; R₁, L₁, and L₂ are defined the same as in claim 1.

23. The conjugate compound of claim 1, wherein the conjugate compound is selected from structures of Tx01, Tx02 and Tx03 as following: 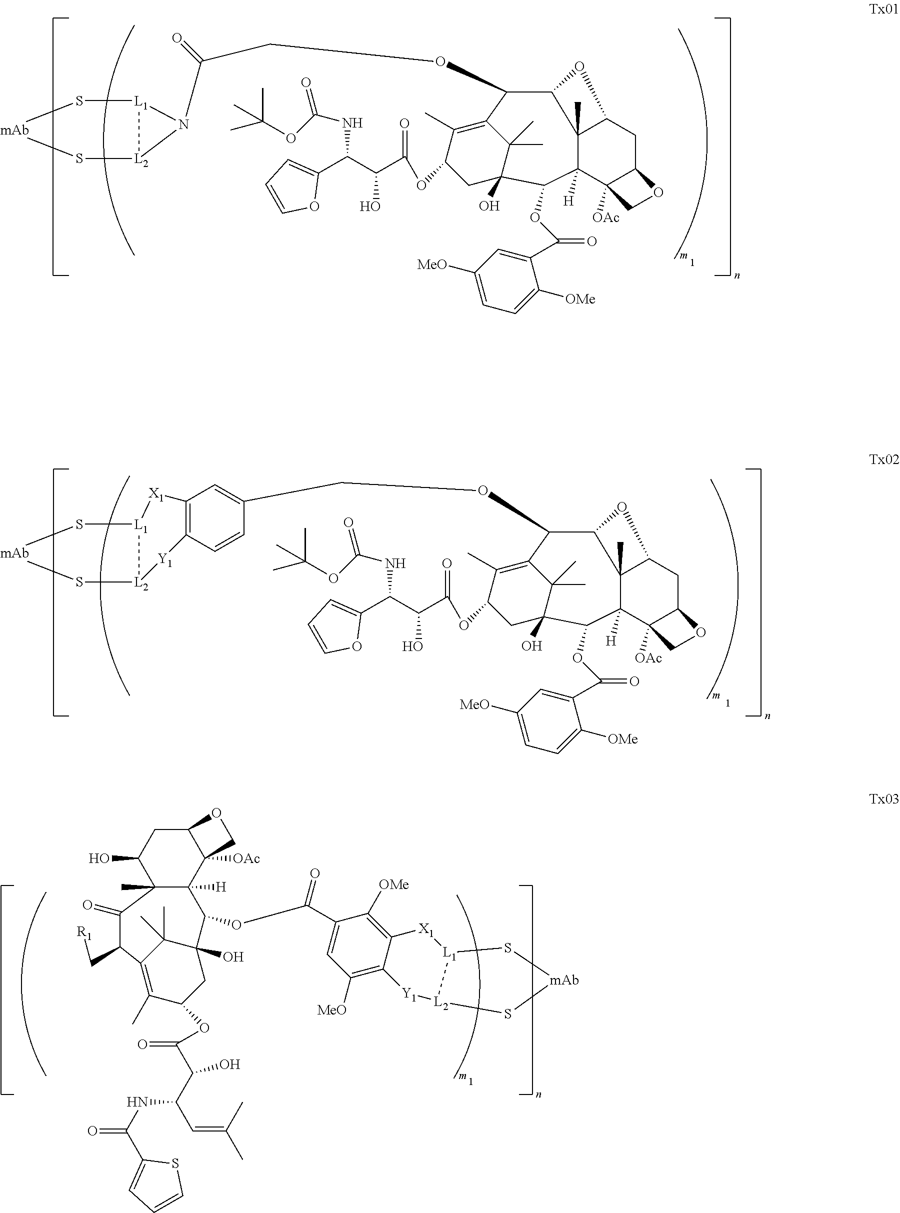
wherein “-----” is a single bond, a double bond, or absent; X₁, and Y₁ are independently O, NH, NHNH, NR₅, S, C(O)O, C(O)NH, OC(O)NH, OC(O)O, NHC(O)NH, NHC(O)S, OC(O)N(R₁), N(R₁)C(O)N(R₁), CH, C(O)NHNHC(O) or C(O)NR₁; mAb is antibody; n and m₁ are independently 1-20; R₁, L₁, and L₂ are defined the same as in claim 1.

24. The conjugate compound of claim 1, wherein the conjugate compound is selected from structures of CC01, CC02, and CC03 as following: 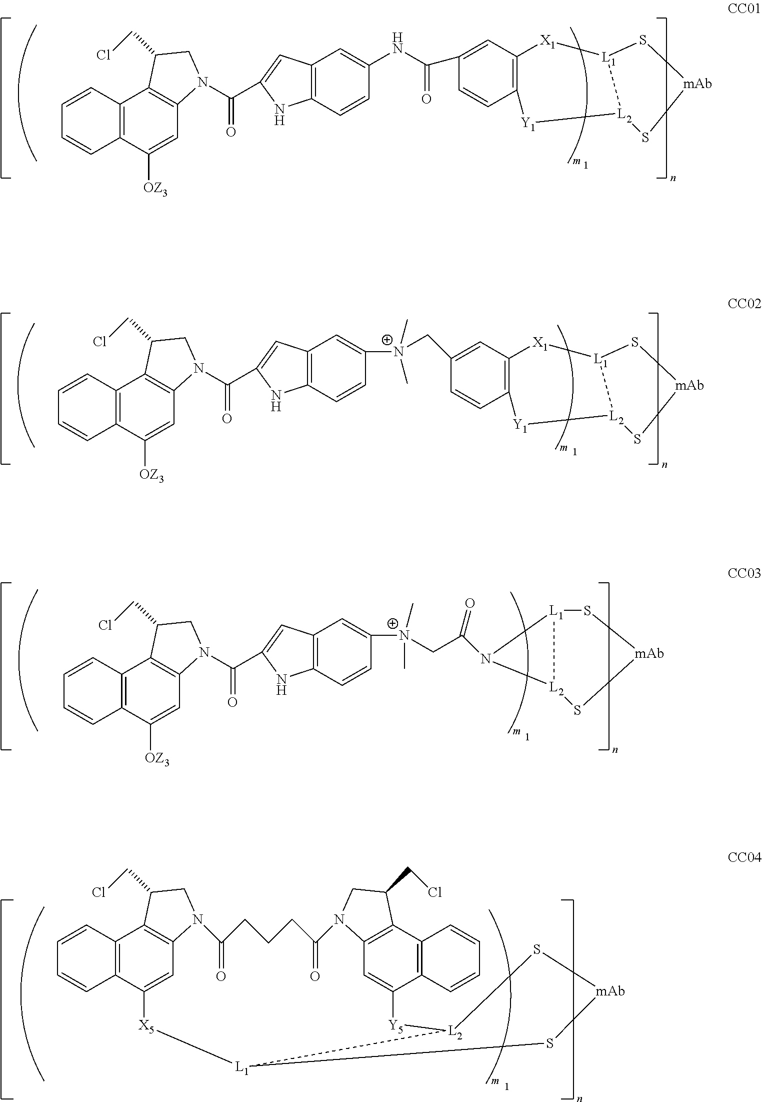
wherein mAb is an antibody; Z₃ is H, PO(OM₁)(OM₂), SO₃M₁, CH₂PO(OM₁)(OM₂), CH₃N(CH₂CH₂)₂NC(O)—, O(CH₂CH₂)₂NC(O)—, R₁, or glycoside; wherein “-----” is a single bond, a double bond, or absent; X₁, X₅, Y₁ and Y₅ are independently O, NH, NHNH, NR₅, S, C(O)O, C(O)NH, OC(O)NH, OC(O)O, NHC(O)NH, NHC(O)S, OC(O)N(R₁), N(R₁)C(O)N(R₁), CH, C(O)NHNHC(O) or C(O)NR₁; mAb is antibody; n and m₁ are independently 1-20; R₁, L₁, and L₂ are defined the same as in claim 1.

25. The conjugate compound of claim 1, wherein the conjugate compound is selected from structures of Da01, Da02, Da03 Da04, Da05, Da06, Da07 and Da08 as following: 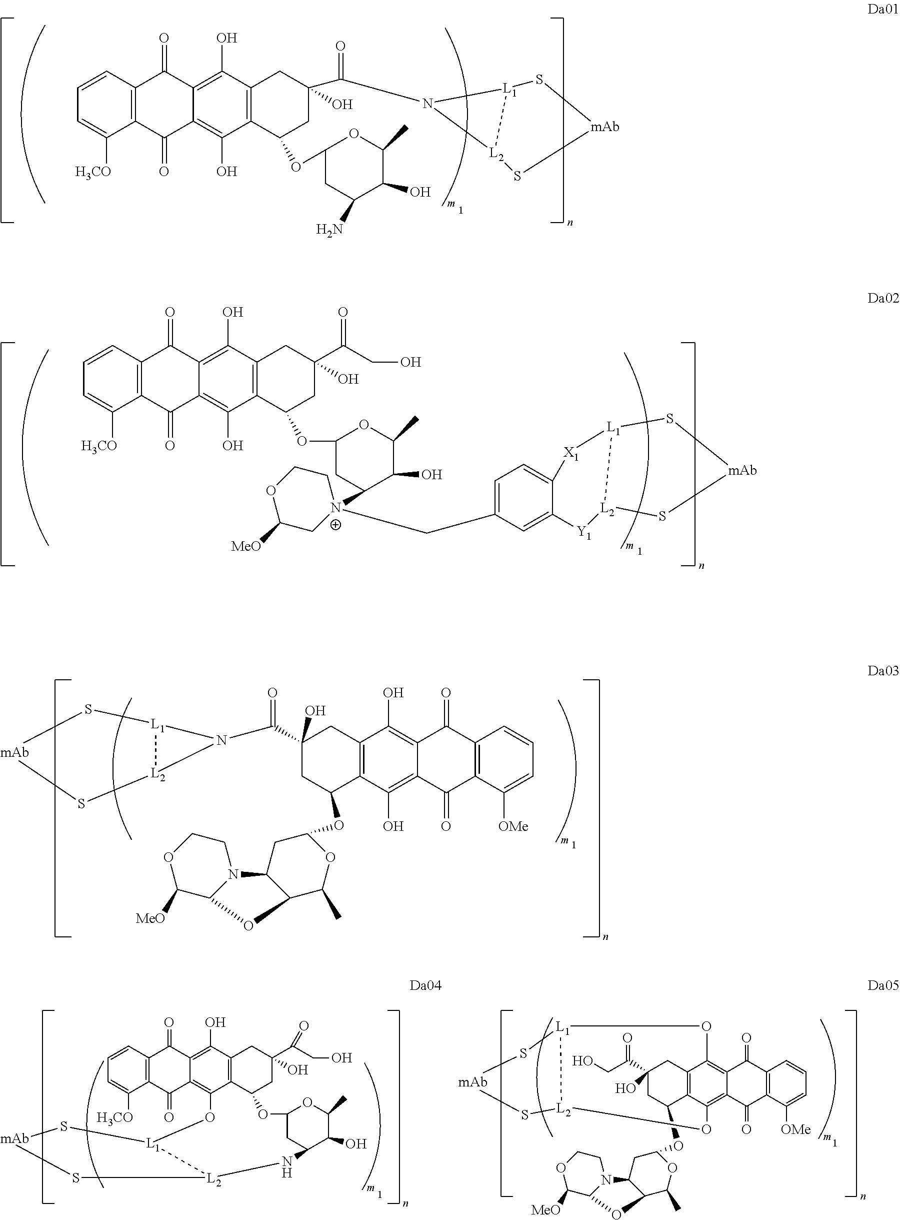 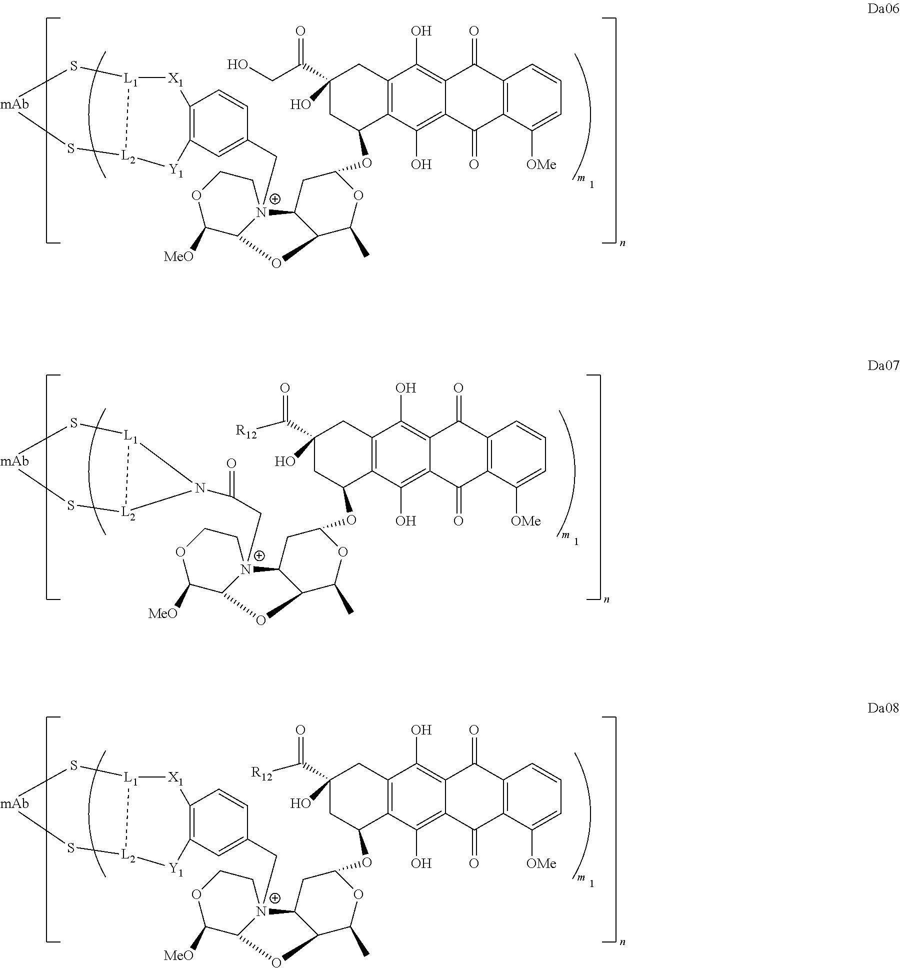
wherein “-----” is a single bond, a double bond, or absent; X₁, and Y₁ are independently O, NH, NHNH, NR₅, S, C(O)O, C(O)NH, OC(O)NH, OC(O)O, NHC(O)NH, NHC(O)S, OC(O)N(R₁), N(R₁)C(O)N(R₁), CH, C(O)NHNHC(O) or C(O)NR₁; R₁₂ is OH, NH₂, NHR₁, NHNH₂, NHNHCOOH, O—R₁—COOH, NH—R₁—COOH, NH-(Aa)ₙCOOH, O(CH₂CH₂O)ₚCH₂CH₂OH, O(CH₂CH₂O)ₚCH₂CH₂NH₂, NH(CH₂CH₂O)ₚCH₂CH₂NH₂, NR₁R₁′, NHOH, NHOR₁, O(CH₂CH₂O)ₚCH₂CH₂COOH, NH(CH₂CH₂O)ₚCH₂CH₂COOH, NH—Ar—COOH, NH—Ar—NH₂, O(CH₂CH₂O)ₚCH₂CH₂NHSO₃H, NH(CH₂CH₂O)ₚCH₂CH₂NH—SO₃H, R₁—NHSO₃H, NH—R₁—NHSO₃H, O(CH₂CH₂O)ₚCH₂CH₂NHPO₃H₂, NH(CH₂CH₂O)ₚCH₂—CH₂NHPO₃H₂, OR₁, R₁—NHPO₃H₂, R₁—OPO₃H₂, O(CH₂CH₂O)ₚCH₂CH₂OPO₃H₂, OR₁—NHPO₃H₂, NH—R₁—NHPO₃H₂, NH(CH₂CH₂NH)ₚCH₂CH₂NH₂, NH(CH₂CH₂S)ₚCH₂CH₂NH₂, NH(CH₂CH₂NH)ₚCH₂CH₂OH, NH(CH₂CH₂S)ₚCH₂CH₂OH, NH—R₁—NH₂, or NH(CH₂CH₂O)ₚCH₂CH₂NHPO₃H₂, wherein Aa is 1-8 aminoacids; p is 1-5000; mAb is antibody; n and m₁ are independently 1-20; R₁, L₁, and L₂ are defined the same as in claim 1.

26. The conjugate compound of claim 1, wherein the conjugate compound is selected from structures of Au01, Au02, Au03, Au04, Au05, Au06, Au07, Au08, Au09, Au10, Au11, Au12 and Au13 as following: 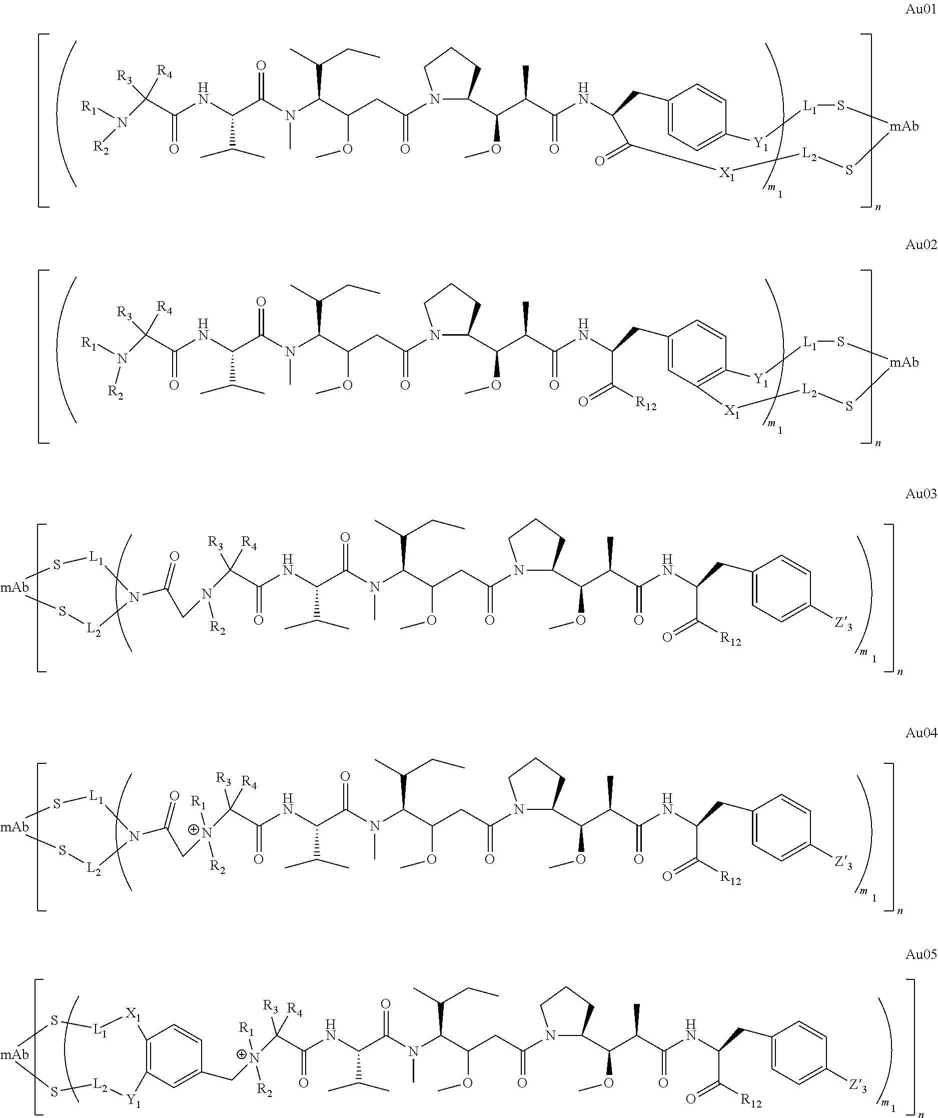 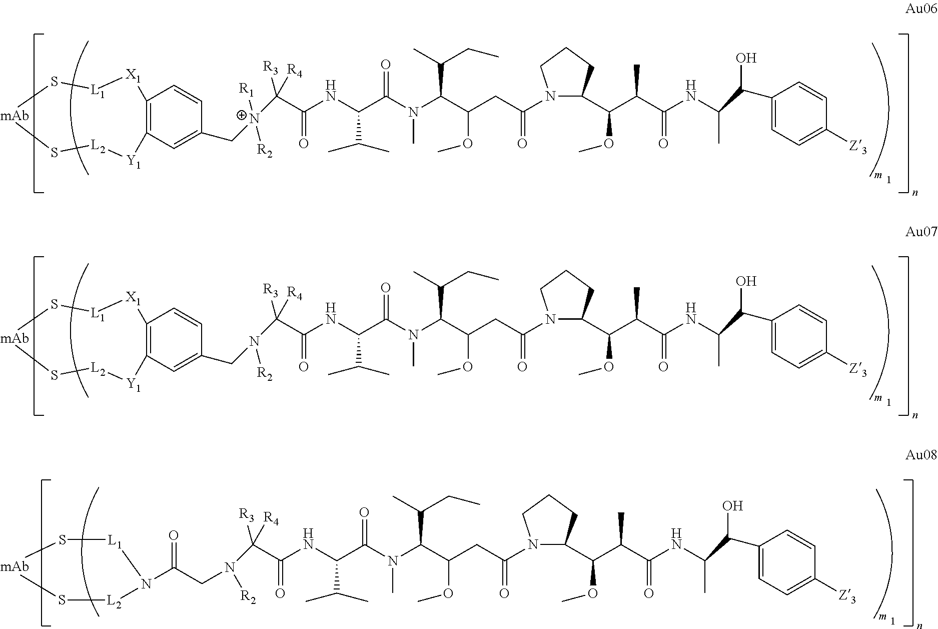 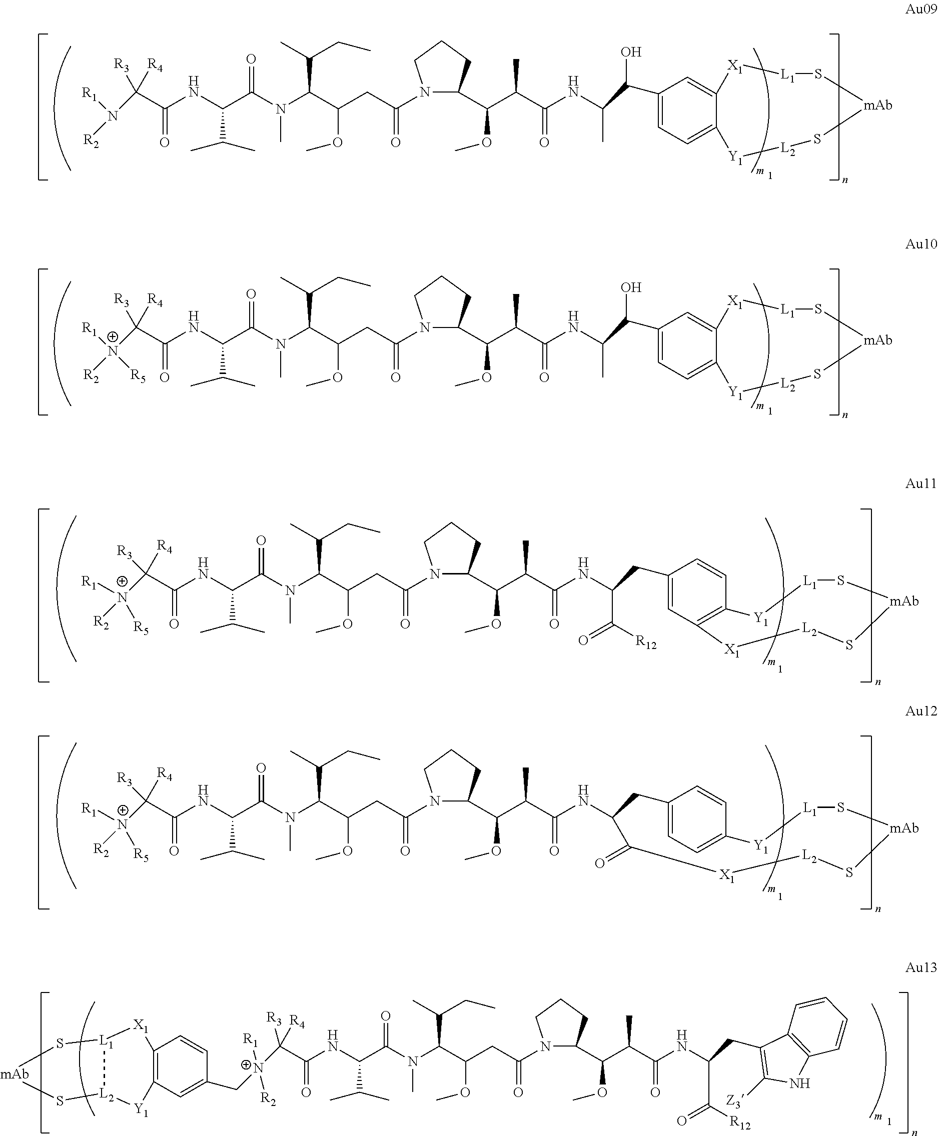
wherein “-----” is a single bond, a double bond, or absent; X₁ and Y₁ are independently O, NH, NHNH, NR₅, S, C(O)O, C(O)NH, OC(O)NH, OC(O)O, NHC(O)NH, NHC(O)S, OC(O)N(R₁), N(R₁)C(O)N(R₁), CH, C(O)NHNHC(O) or C(O)NR₁; R₁₂ is OH, NH₂, NHR₁, NHNH₂, NHNHCOOH, O—R₁—COOH, NH—R₁—COOH, NH-(Aa)ₙCOOH, O(CH₂CH₂O)ₚCH₂CH₂OH, O(CH₂CH₂O)ₚCH₂CH₂NH₂, NH(CH₂CH₂O)ₚCH₂CH₂NH₂, NR₁R₁′, NHOH, NHOR₁, O(CH₂CH₂O)ₚCH₂CH₂COOH, NH(CH₂CH₂O)ₚCH₂CH₂COOH, NH—Ar—COOH, NH—Ar—NH₂, O(CH₂CH₂O)ₚCH₂CH₂NHSO₃H, NH(CH₂CH₂O)ₚCH₂CH₂NH—SO₃H, R₁—NHSO₃H, NH—R₁—NHSO₃H, O(CH₂CH₂O)ₚCH₂CH₂NHPO₃H₂, NH(CH₂CH₂O)ₚCH₂—CH₂NHPO₃H₂, OR₁, R₁—NHPO₃H₂, R₁—OPO₃H₂, O(CH₂CH₂O)ₚCH₂CH₂OPO₃H₂, OR₁—NHPO₃H₂, NH—R₁—NHPO₃H₂, NH(CH₂CH₂NH)ₚCH₂CH₂NH₂, NH(CH₂CH₂S)ₚCH₂CH₂NH₂, NH(CH₂CH₂NH)ₚCH₂CH₂OH, NH(CH₂CH₂S)ₚCH₂CH₂OH, NH—R₁—NH₂, or NH(CH₂CH₂O)ₚCH₂CH₂NHPO₃H₂, wherein Aa is 1-8 aminoacids; p is 1-5000; mAb is antibody; n and m₁ are independently 1-20; p is 1-5000; R₁, R₂, R₃, and R₄ are independently H; C₁-C₈ linear or branched alkyl, aryl, heteroaryl, heteroalkyl, alkylcycloalkyl, ester, ether, amide, amine, heterocycloalkyl, or acyloxylamine; or peptide containing 1-8 aminoacids, or polyethyleneoxy unit having formula (OCH₂CH₂)ₚ or (OCH₂CH(CH₃))ₚ, wherein p is an integer from 1 to about 5000; or two Rs: R₁R₂, R₂R₃, R₁R₃ or R₃R₄ form 3˜8 member cyclic ring of alkyl, aryl, heteroaryl, heteroalkyl, or alkylcycloalkyl group; X₃ is H, CH₃ or X₁′R₁′, wherein X₁′ is NH, N(CH₃), NHNH, O, or S, and R₁′ is H or C₁-C₈ linear or branched alkyl, aryl, heteroaryl, heteroalkyl, alkylcycloalkyl, acyloxylamines; R_{3′} is H or —C₁-C₆ linear or branched alkyl; Z₃′ is H, COOR₁, NH₂, NHR₁, OR₁, CONHR₁, NHCOR₁, OCOR₁, OP(O)(OM₁)(OM₂), OCH₂OP(O)(OM₁)(OM₂), OSO₃M₁, R₁, or O-glycoside (glucoside, galactoside, mannoside, glucuronoside/glucuronide, alloside, fructoside), NH-glycoside, S-glycoside or CH₂-glycoside; M₁ and M₂ are independently H, Na, K, Ca, Mg, NH₄, or NR₁R₂R₃; L₁, and L₂ are defined the same as in claim 1.

27. The conjugate compound of claim 1, wherein the conjugate compound is selected from structures of PB01, PB02, PB03, PB04, PB05, PB06, PB07, PB08, PB09, PB10, PB11, PB12, PB13, PB14, PB15, PB16, PB17, PB18, PB19, PB20, PB21 and PB22m 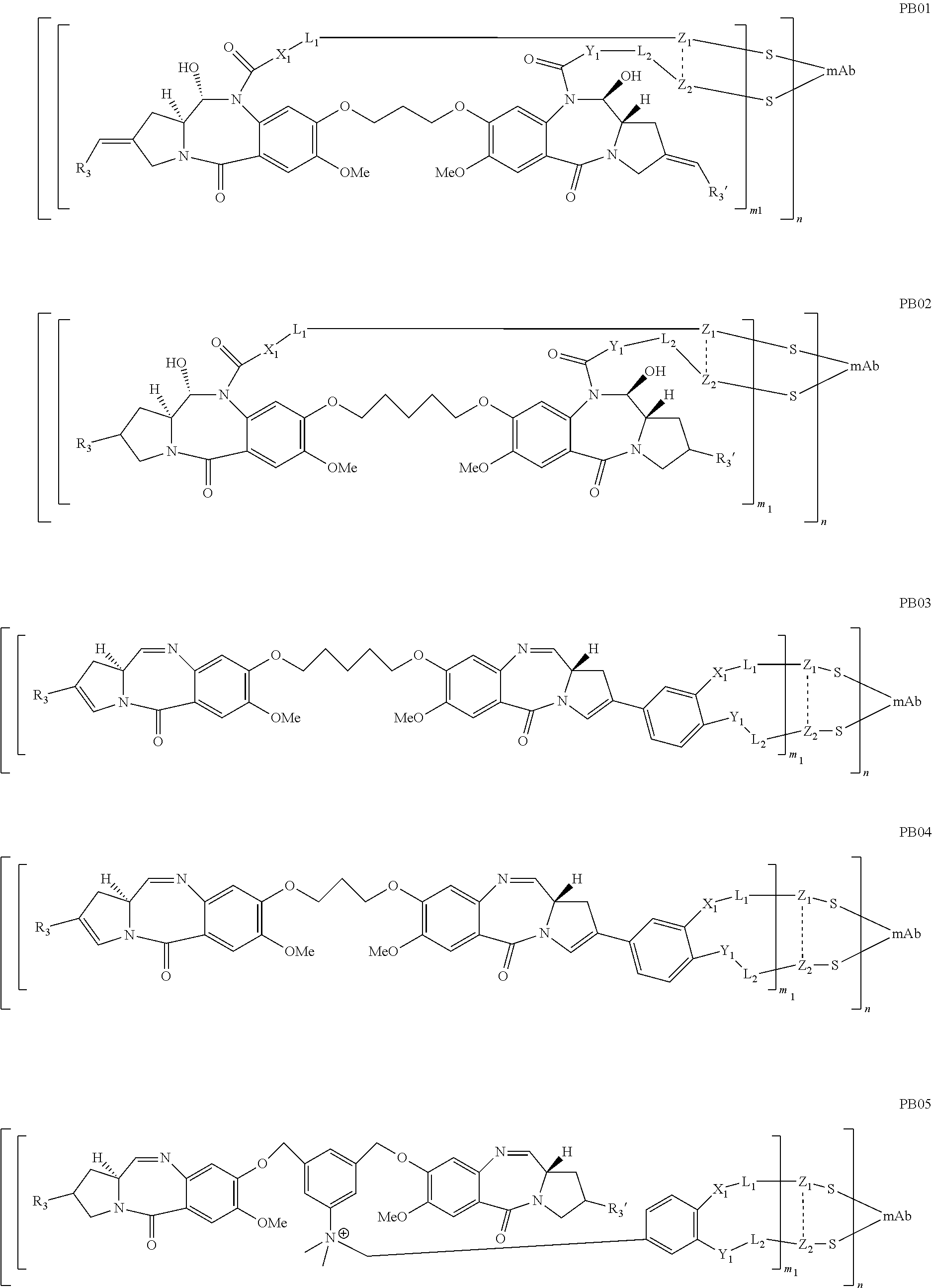 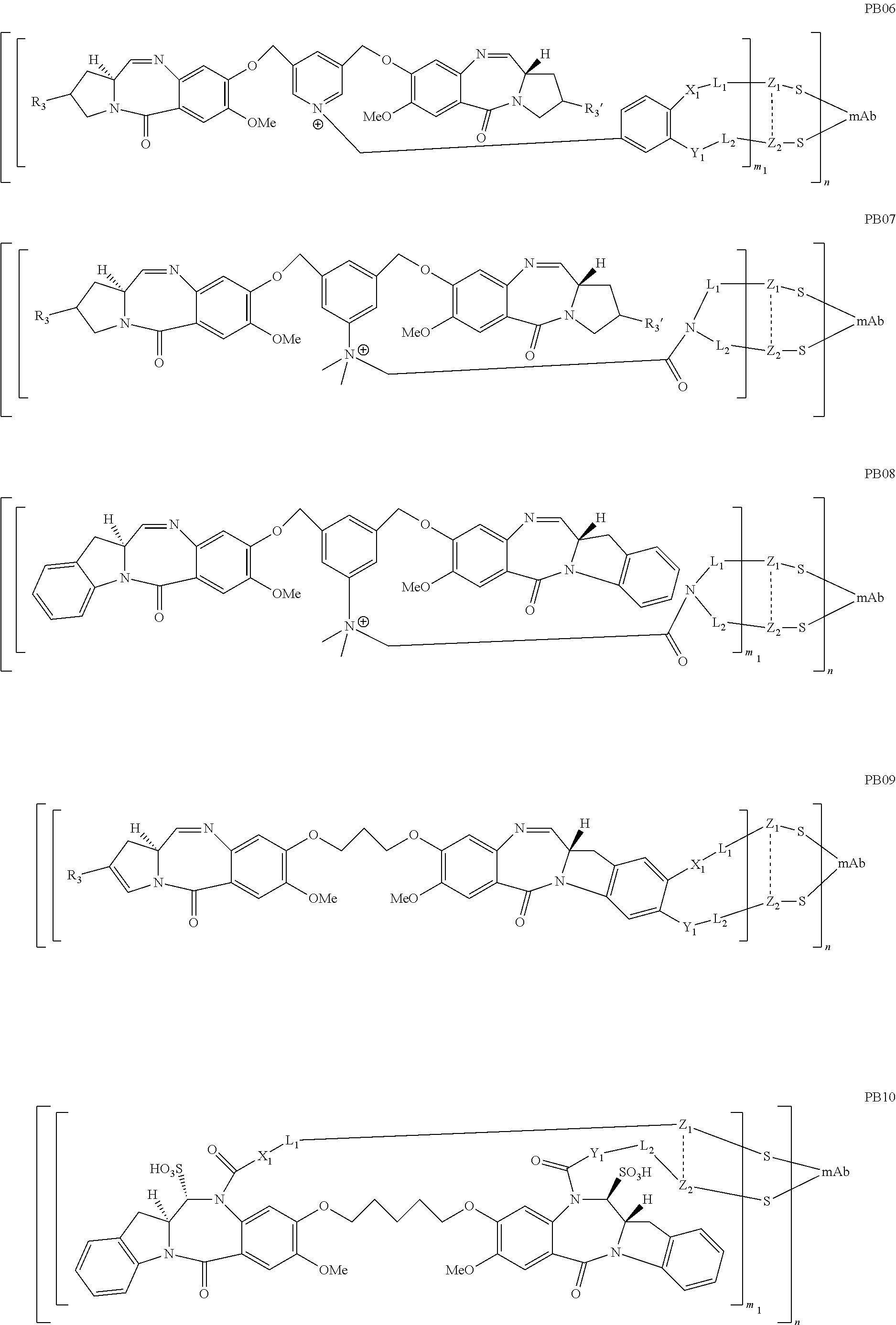 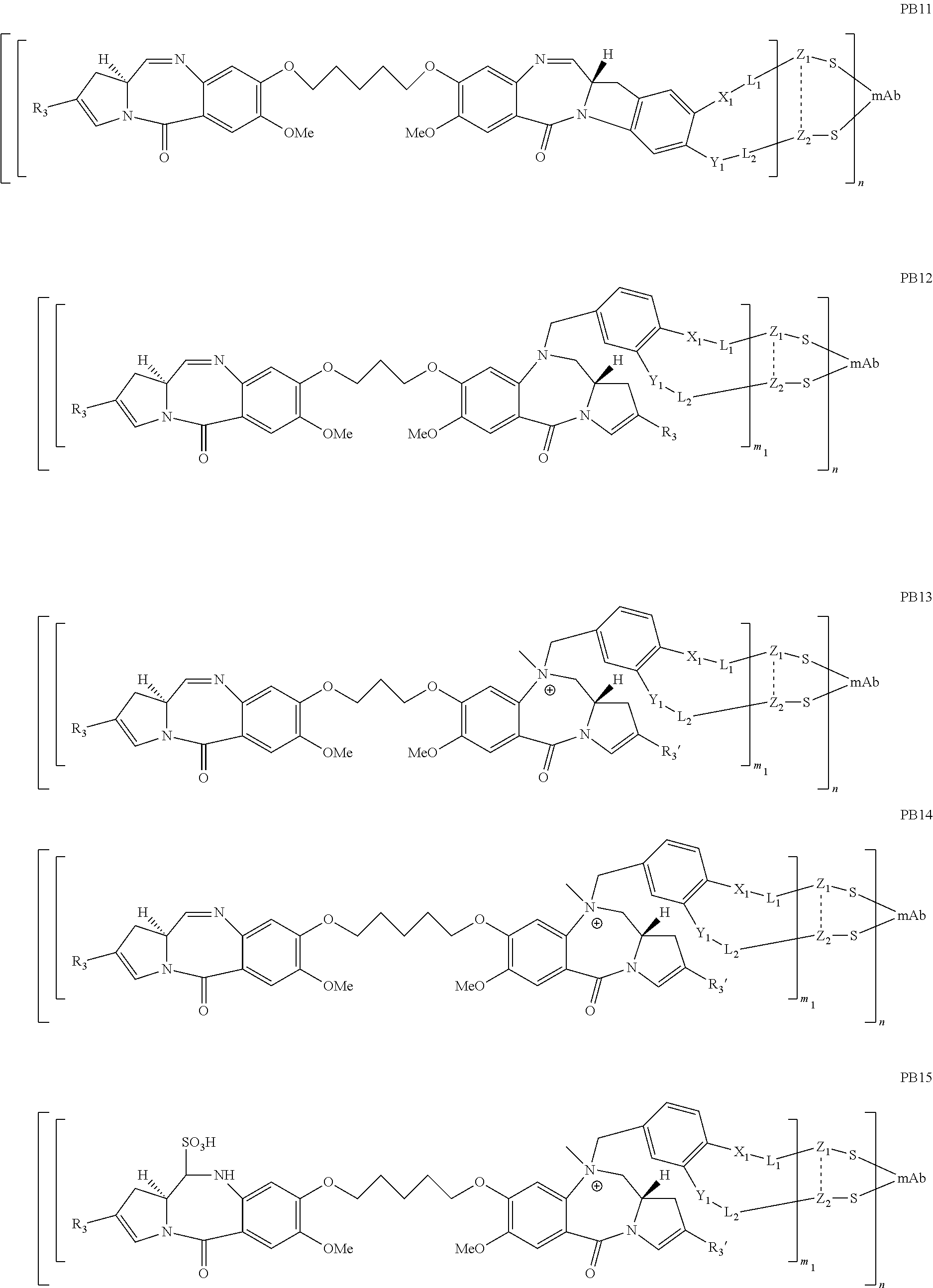 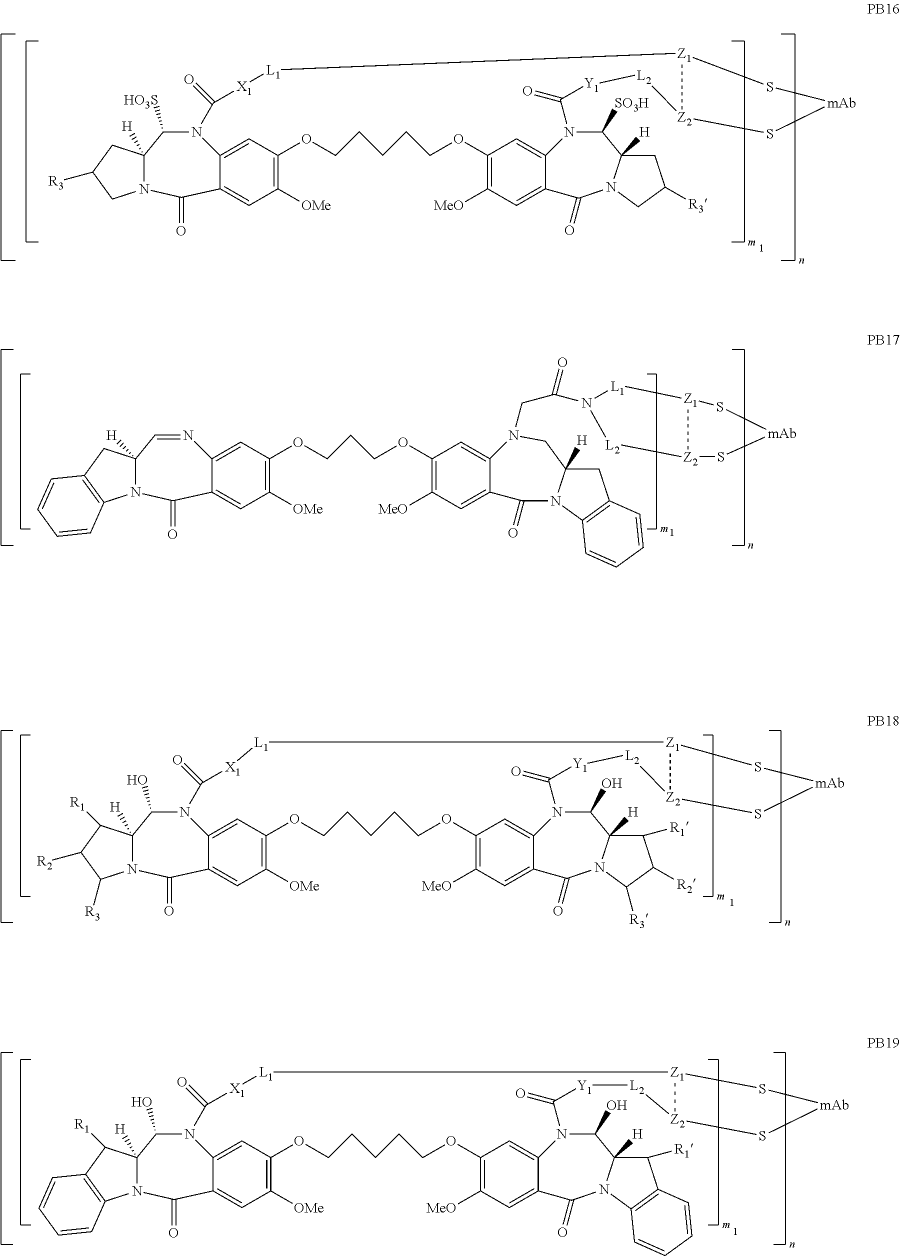 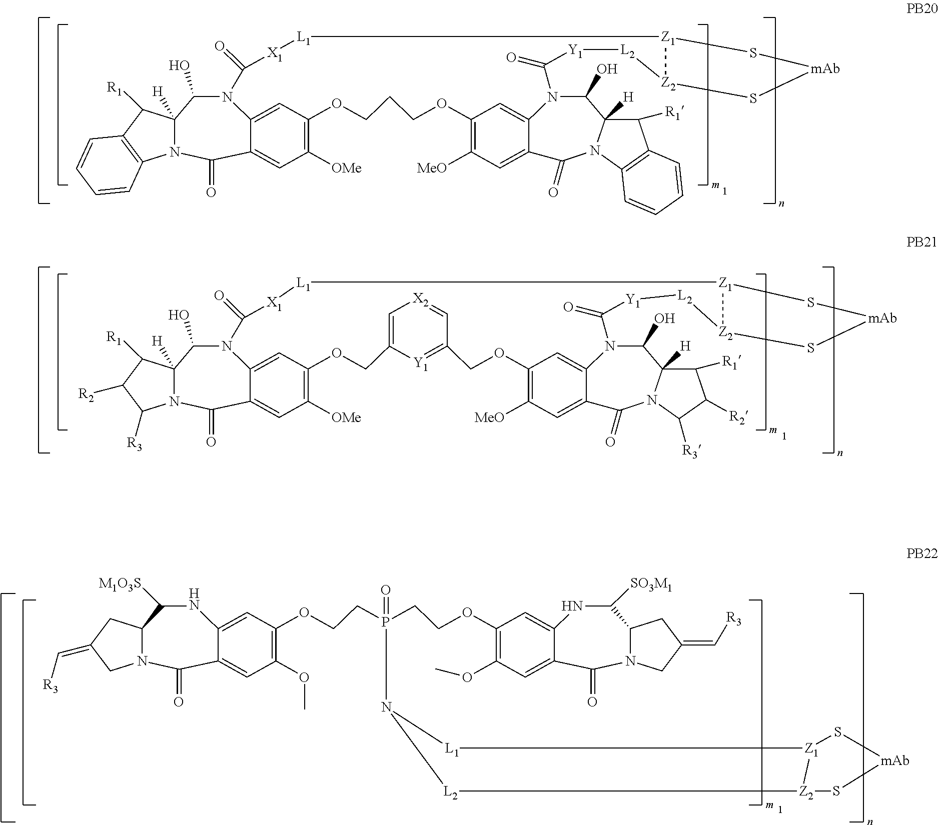
wherein “-----” is optionally either a single bond, or a double bond, or absent; X₁, and Y₁ are independently O, NH, NHNH, NR₅, S, C(O)O, C(O)NH, OC(O)NH, OC(O)O, NHC(O)NH, NHC(O)S, OC(O)N(R₁), N(R₁)C(O)N(R₁), CH, C(O)NHNHC(O) or C(O)NR₁; mAb is antibody; n and m₁ are independently 1-20; L₁, L₂, Z₁, and Z₂, are defined the same as in claim 1; R₁, R₂, R₃, R₁′, R₂′, and R_{3′} are independently H; F; Cl; ═O; ═S; OH; SH; C₁-C₈ linear or branched alkyl, aryl, alkenyl, L-heteroaryl, heteroalkyl, alkylcycloalkyl, ester (COOR₅ or —OC(O)R₅), ether (OR₅), amide (CONR₅), carbamate (OCONR₅), amine (NHR₅, NR₅R₅′), heterocycloalkyl, or acyloxylamine (—C(O)NHOH, —ONHC(O)R₅); or peptide containing 1-8 natural or unnatural aminoacids, or polyethyleneoxy unit of formula (OCH₂CH₂)ₚ or (OCH₂CH(CH₃))ₚ, wherein p is an integer from 1 to about 5000; or two Rs: R₁R₂, R₂R₃, R₁R₃, R₁′R₂′, R₂′R₃′, or R₁′R₃′ independently form 3˜8 member cyclic ring of alkyl, aryl, heteroaryl, heteroalkyl, or alkylcycloalkyl group; X₂ and Y₂ are independently N, CH₂ or CR₅, wherein R₅ is H, OH, NH₂, NH(CH₃), NHNH₂, COOH, SH, OZ₃, SZ₃, or C₁-C₈ linear or branched alkyl, aryl, heteroaryl, heteroalkyl, alkylcycloalkyl, acyloxylamines; Z₃ is H, OP(O)(OM₁)(OM₂), OCH₂OP(O)(OM₁)(OM₂), OSO₃M₁, or O-glycoside (glucoside, galactoside, mannoside, glucuronoside/glucuronide, alloside, fructoside), NH-glycoside, S-glycoside or CH₂-glycoside; M₁ and M₂ are independently H, Na, K, Ca, Mg, NH₄, or NR₁R₂R₃.

28. The conjugate compound of claim 1, wherein the conjugate compound is selected from structures of Am01, Am02, Am03, and Am04 below: 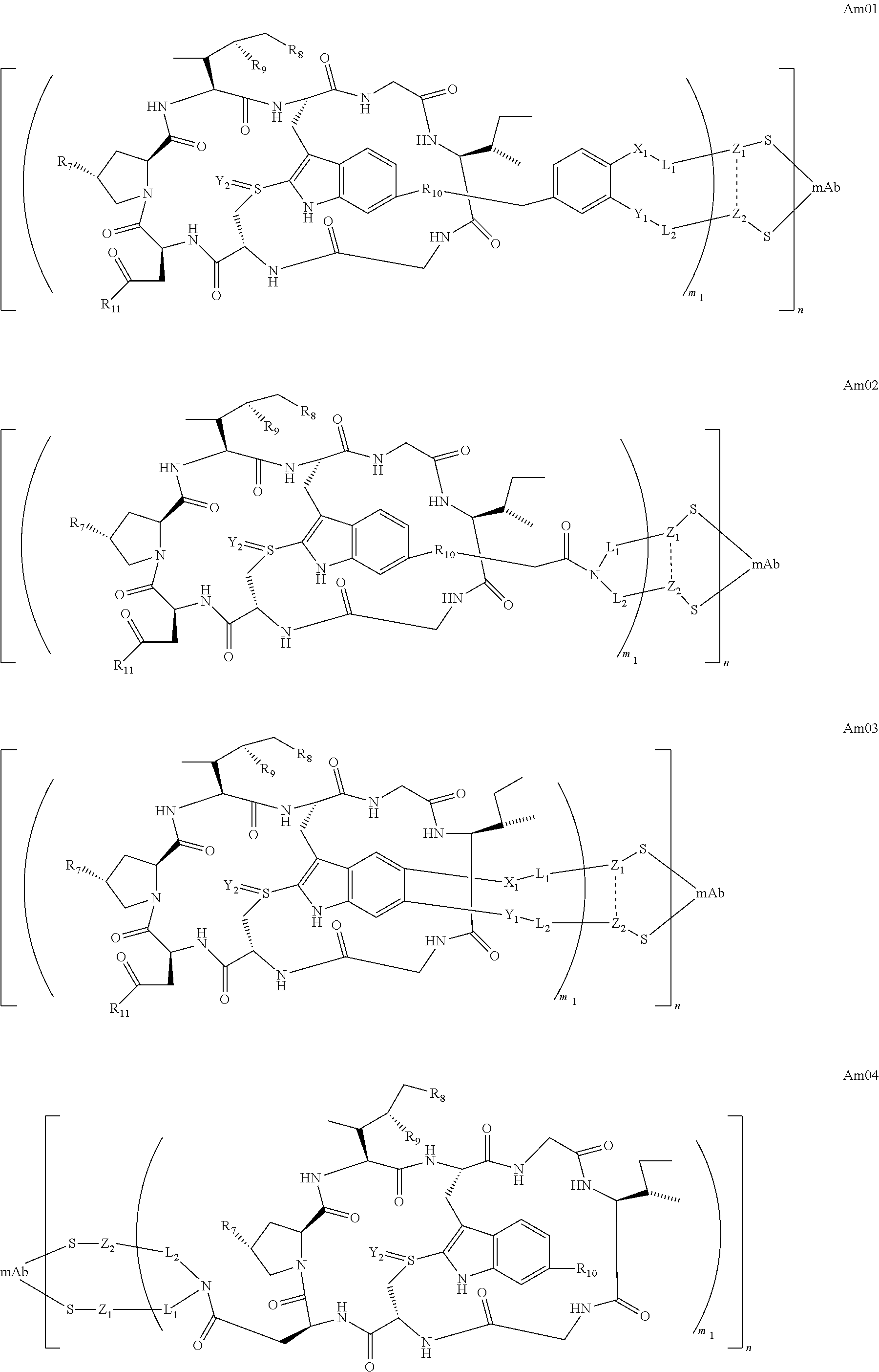
wherein “-----” is a single bond, a double bond, or absent; X₁, and Y₁ are --independently O, NH, NHNH, NR₅, S, C(O)O, C(O)NH, OC(O)NH, OC(O)O, NHC(O)NH, NHC(O)S, OC(O)N(R₁), N(R₁)C(O)N(R₁), CH, C(O)NHNHC(O) or C(O)NR₁; mAb is antibody; n and -m₁ are independently 1-20; R₇, R₈, and R₉ are independently H, OH, OR₁, NH₂, NHR₁, C₁-C₆ alkyl, or absent; Y₂ is O, O₂, NR₁, NH, or absent; R₁₀ is CH₂, O, NH, NR₁, NHC(O), NHC(O)NH, NHC(O)O, OC(O)O, C(O), OC(O), OC(O)(NR₁), (NR₁)C(O)(NR₁), C(O)R₁ or absent; R₁₁ is OH, NH₂, NHR₁, NHNH₂, NHNHCOOH, O—R₁—COOH, NH—R₁—COOH, NH-(Aa)ₙCOOH, O(CH₂CH₂O)ₚCH₂CH₂OH, O(CH₂CH₂O)ₚCH₂CH₂NH₂, NH(CH₂CH₂O)ₚCH₂CH₂NH₂, NR₁R₁′, O(CH₂CH₂O)ₚCH₂CH₂COOH, NH(CH₂CH₂O)ₚCH₂CH₂COOH, NH—Ar—COOH, NH—Ar—NH₂, O(CH₂CH₂O)ₚCH₂CH₂NHSO₃H, NH(CH₂CH₂O)ₚCH₂CH₂NHSO₃H, R₁—NHSO₃H, NH—R₁—NHSO₃H, O(CH₂CH₂O)ₚCH₂CH₂NHPO₃H₂, NH(CH₂CH₂O)ₚCH₂CH₂NHPO₃H₂, OR₁, R₁—NHPO₃H₂, R₁—OPO₃H₂, O(CH₂CH₂O)ₚCH₂CH₂OPO₃H₂, OR₁—NHPO₃H₂, NH—R₁—NHPO₃H₂, or NH(CH₂CH₂O)ₚCH₂CH₂NHPO₃H₂, wherein Aa is 1-8 aminoacids; n and m₁ are independently 1-20; p is 1-5000; R₁, L₁, L₂, Z₁, and Z₂, are defined the same as in claim 1.

29. The conjugate compound of claim 1, wherein the conjugate compound is selected from structures of Pg01, Pg02, and Pg03: 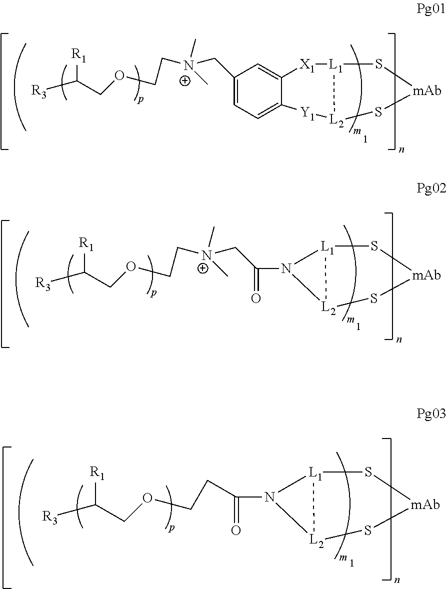
wherein “-----” is a single bond, a double bond, or absent; X₁, and Y₁ are independently O, NH, NHNH, NR₅, S, C(O)O, C(O)NH, OC(O)NH, OC(O)O, NHC(O)NH, NHC(O)S, OC(O)N(R₁), N(R₁)C(O)N(R₁), CH, C(O)NHNHC(O) or C(O)NR₁; mAb is antibody; n and m₁ are independently 1-20; p is 1-5000; R₁ and R₃ are H, OH, OCH₃, CH₃, or OC₂H₅ independently; L₁, and L₂ are defined the same as in claim 1.

30. The conjugate compound of claim 1, wherein the conjugate compound is selected from structures of: LB01 (Folate conjugate), LB02 (PMSA ligand conjugate), LB03 (PMSA ligand conjugate), LB04 (PMSA ligand conjugate), LB05 (Somatostatin conjugate), LB06 (Somatostatin conjugate), LB07 (Octreotide, a Somatostatin analog conjugate), LB08 (Lanreotide, a Somatostatin analog conjugate), LB09 (Vapreotide (Sanvar), a Somatostatin analog conjugate), LB10 (CAIX ligand conjugate), LB11 (CAIX ligand conjugate), LB12 (Gastrin releasing peptide receptor (GRPr), MBA conjugate), LB13 (luteinizing hormone-releasing hormone (LH-RH) ligand and GnRH conjugate), LB14 (luteinizing hormone-releasing hormone (LH-RH) and GnRH ligand conjugate), LB15 (GnRH antagonist, Abarelix conjugate), LB16 (cobalamin, vitamin B12 analog conjugate), LB17 (cobalamin, vitamin B12 analog conjugate), LB18 (for α_{V}β₃ integrin receptor, cyclic RGD pentapeptide conjugate), LB19 (hetero-bivalent peptide ligand conjugate for VEGF receptor), LB20 (Neuromedin B conjugate), LB21 (bombesin conjugate for a G-protein coupled receptor), LB22 (TLR₂ conjugate for a Toll-like receptor,), LB23 (for an androgen receptor), LB24 (Cilengitide/cyclo(-RGDfV-) conjugate for an αᵥ intergrin receptor, LB23 (Fludrocortisone conjugate), LB25 (Rifabutin analog conjugate), LB26 (Rifabutin analog conjugate), LB27 (Rifabutin analog conjugate), LB28 (Fludrocortisone conjugate), LB29 (Dexamethasone conjugate), LB30 (fluticasone propionate conjugate), LB31 (Beclometasone dipropionate conjugate), LB32 (Triamcinolone acetonide conjugate), LB33 (Prednisone conjugate), LB34 (Prednisolone conjugate), LB35 (Methylprednisolone conjugate), LB36 (Betamethasone conjugate), LB37 (Irinotecan analog conjugate), LB38 (Crizotinib analog conjugate), LB39 (Bortezomib analog conjugate), LB40 (Carfilzomib analog conjugate), LB41 (Carfilzomib analog conjugate), LB42 (Leuprolide analog conjugate), LB43 (Triptorelin analog conjugate), LB44 (Clindamycin conjugate), LB45 (Liraglutide analog conjugate), LB46 (Semaglutide analog conjugate), LB47 (Retapamulin analog conjugate), LB48 (Indibulin analog conjugate), LB49 (Vinblastine analog conjugate), LB50 (Lixisenatide analog conjugate), LB51 (Osimertinib analog conjugate) LB52 (a neucleoside analog conjugate), LB53 (Erlotinib analog conjugate) and LB54 (Lapatinib analog conjugate) which are shown in following structures: 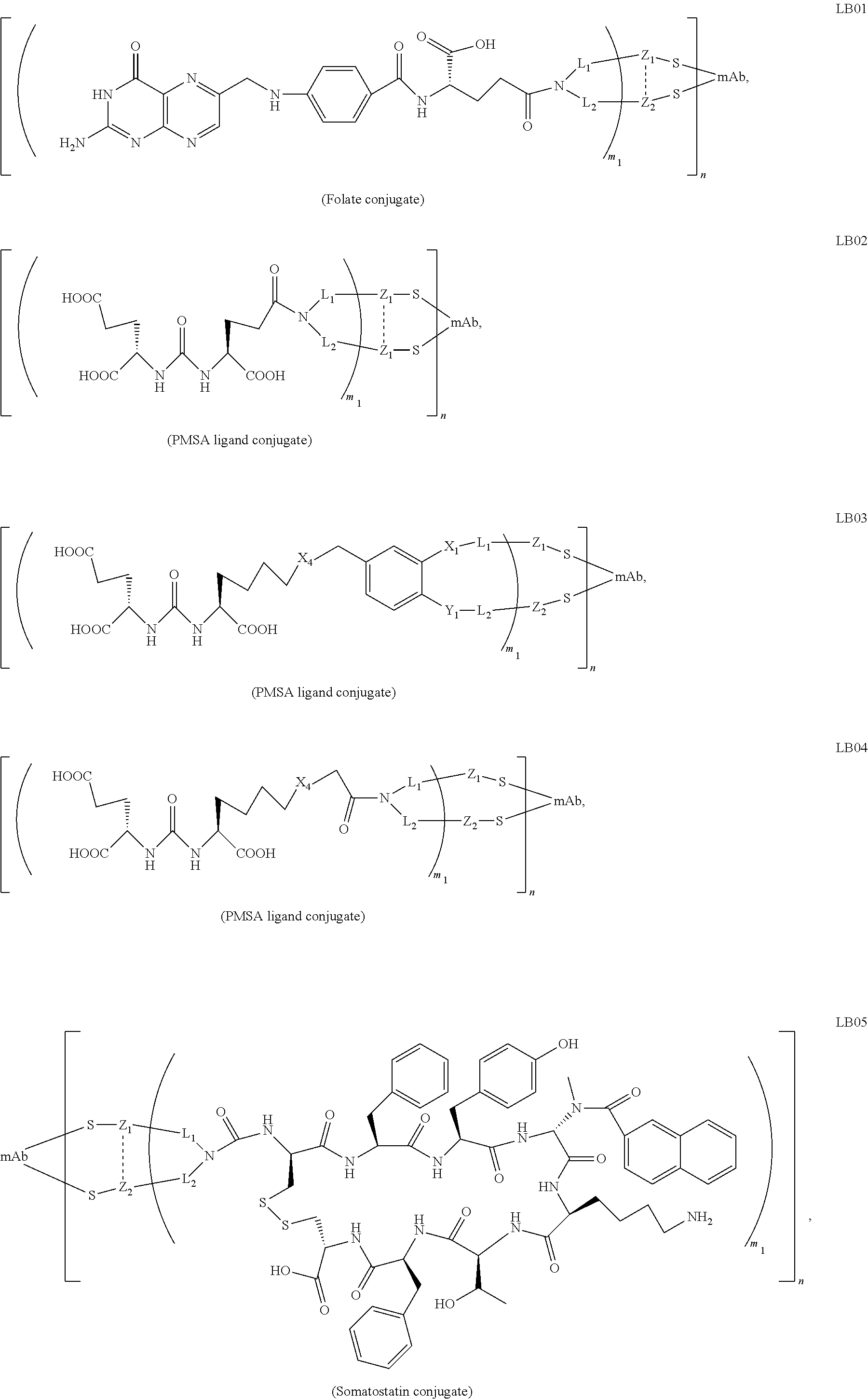 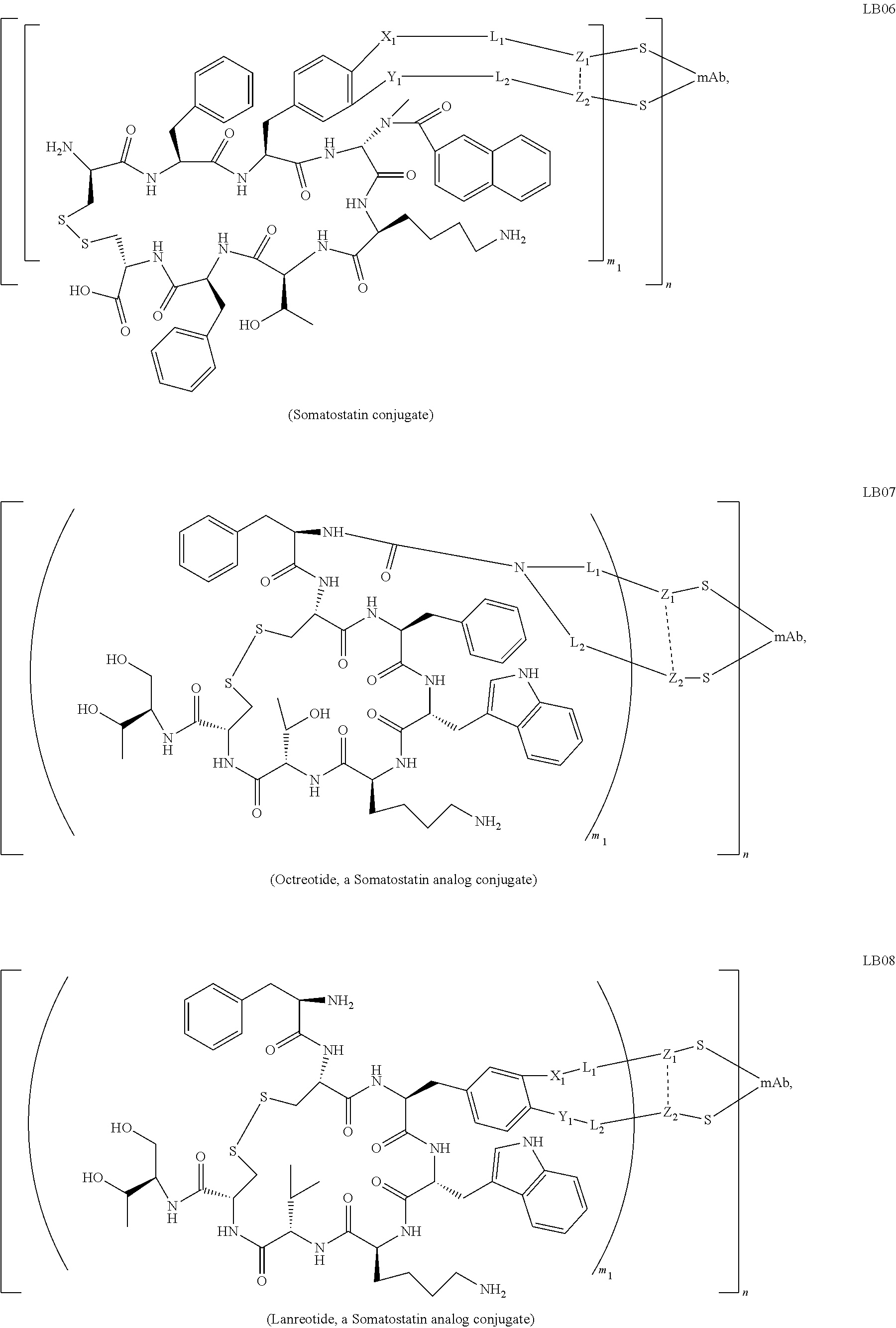 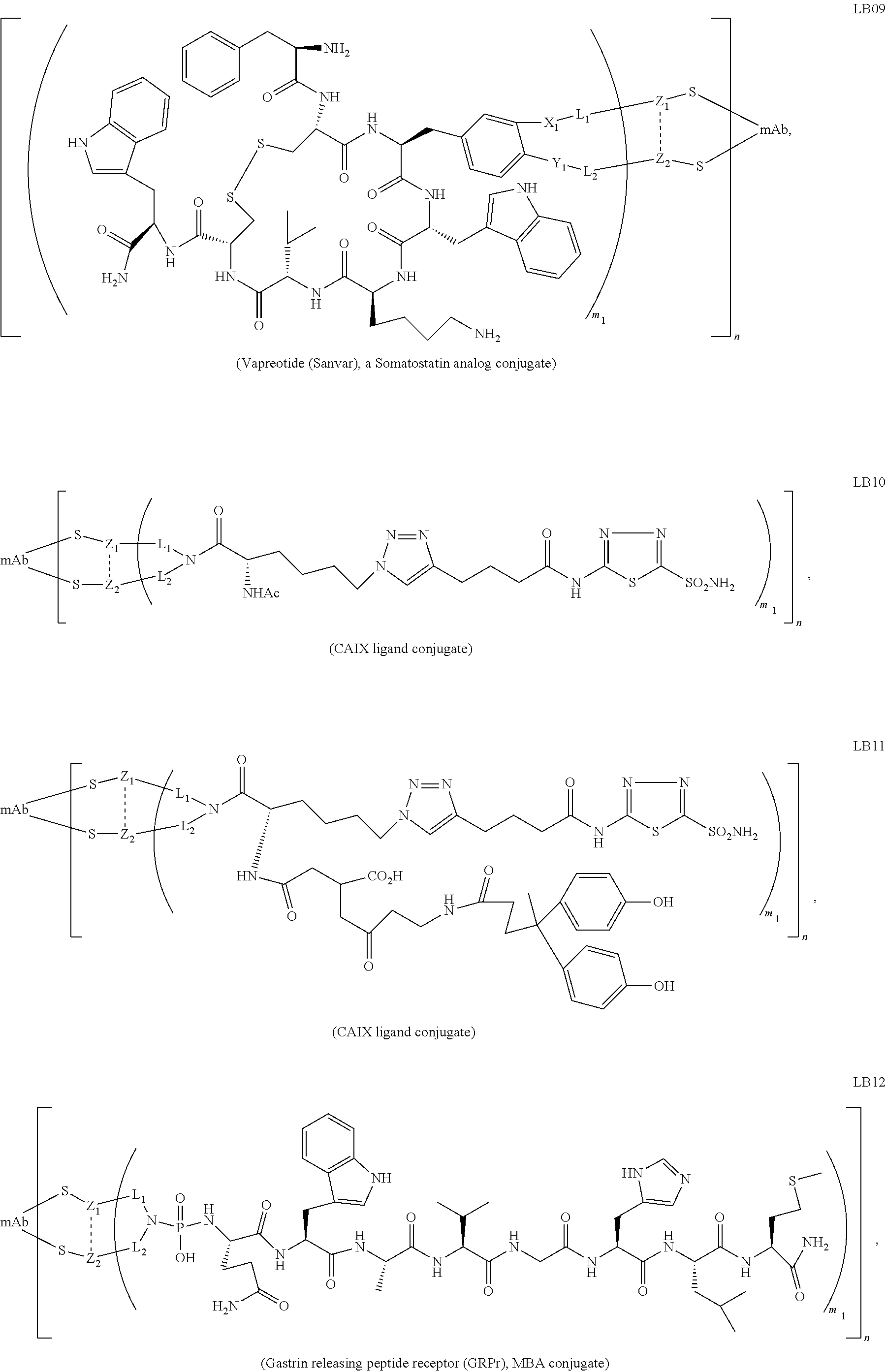 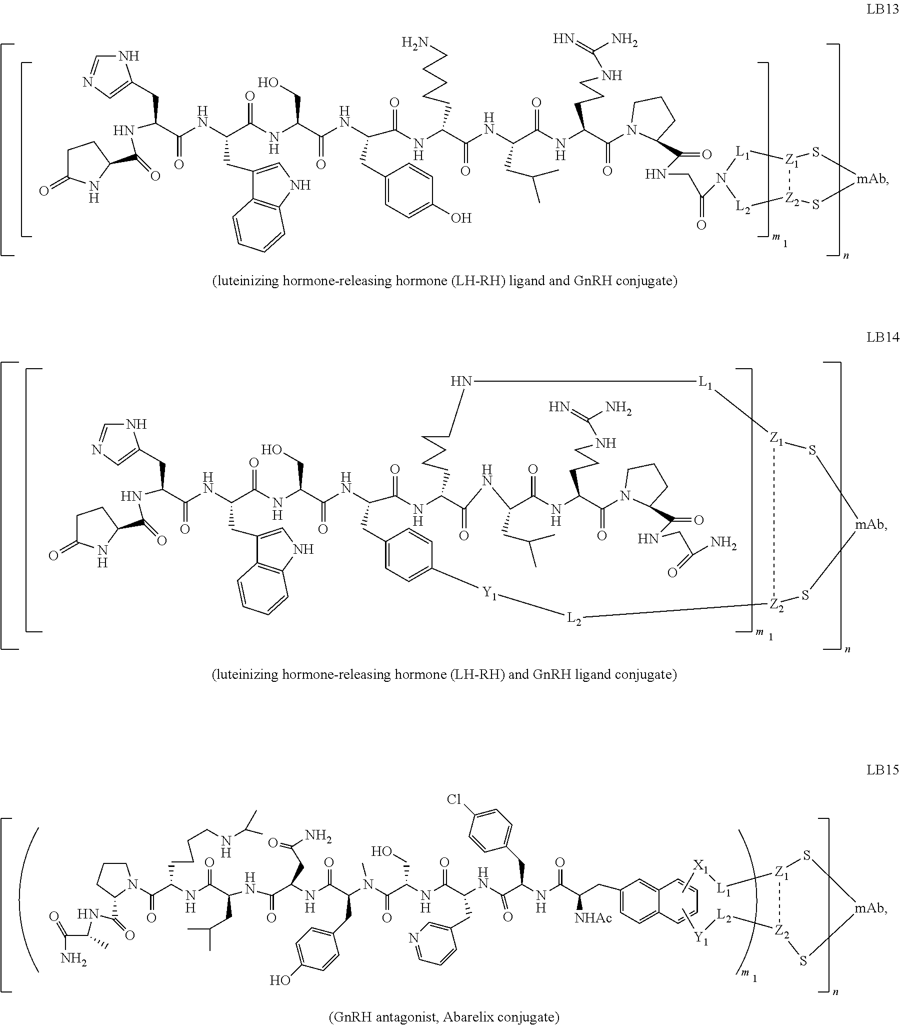 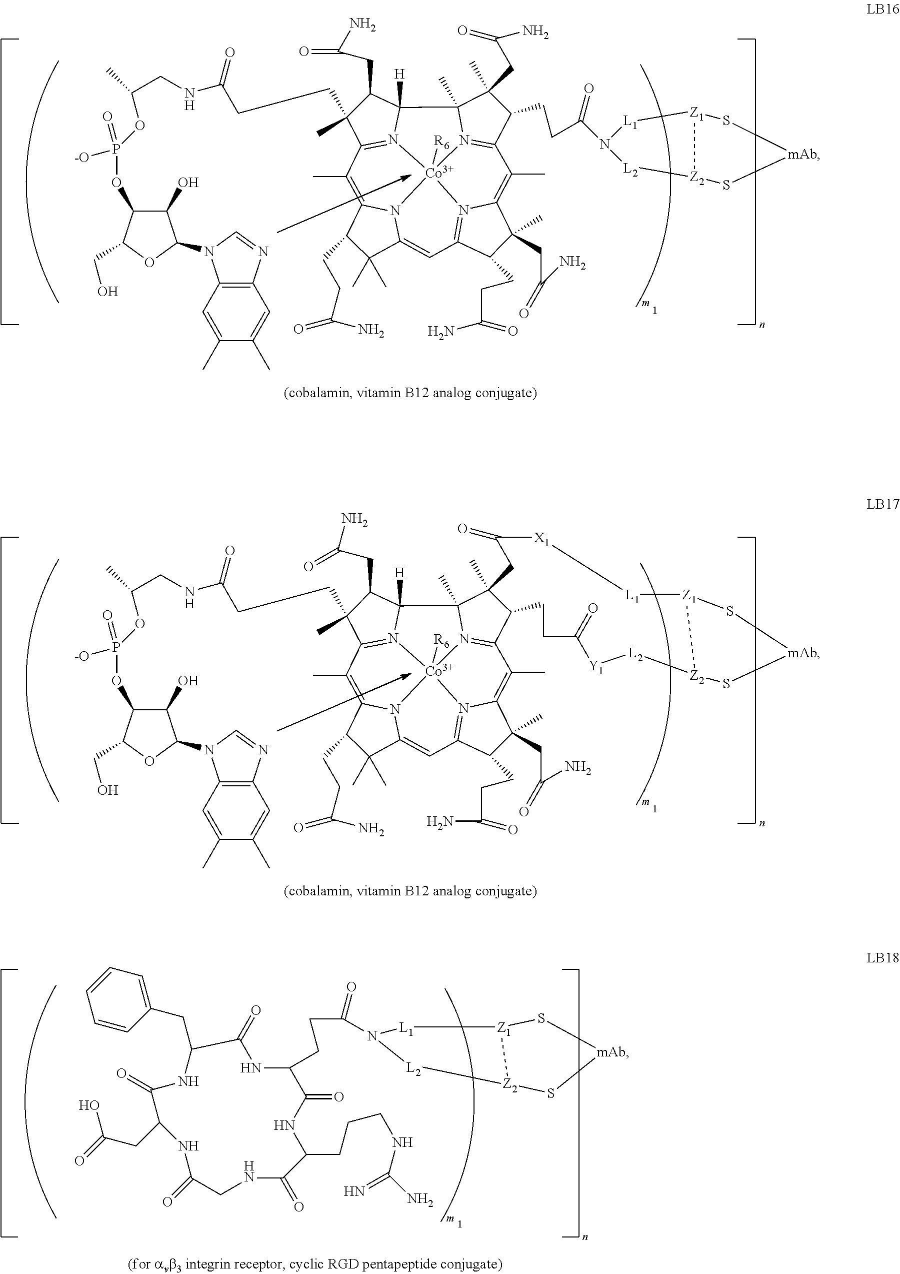 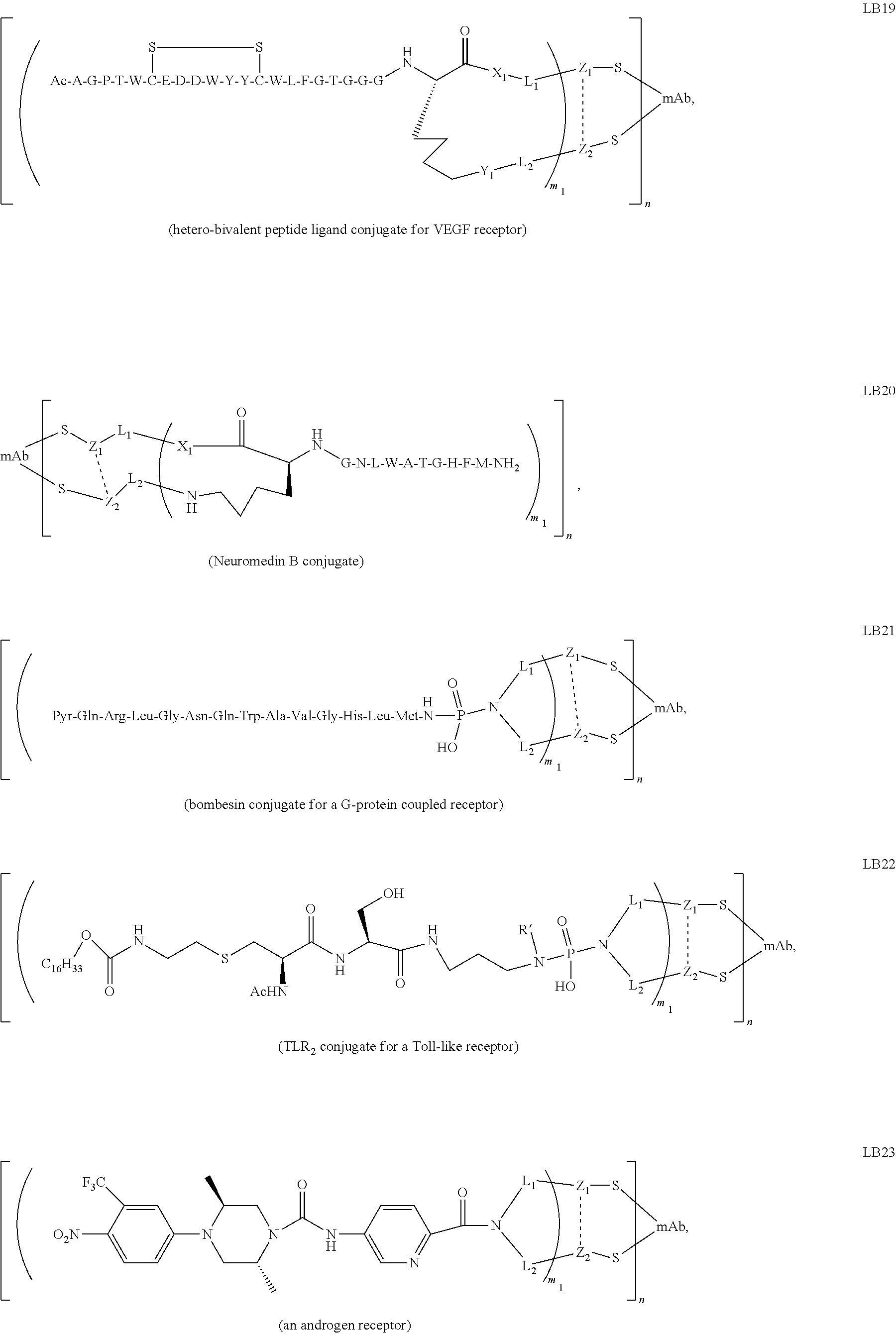 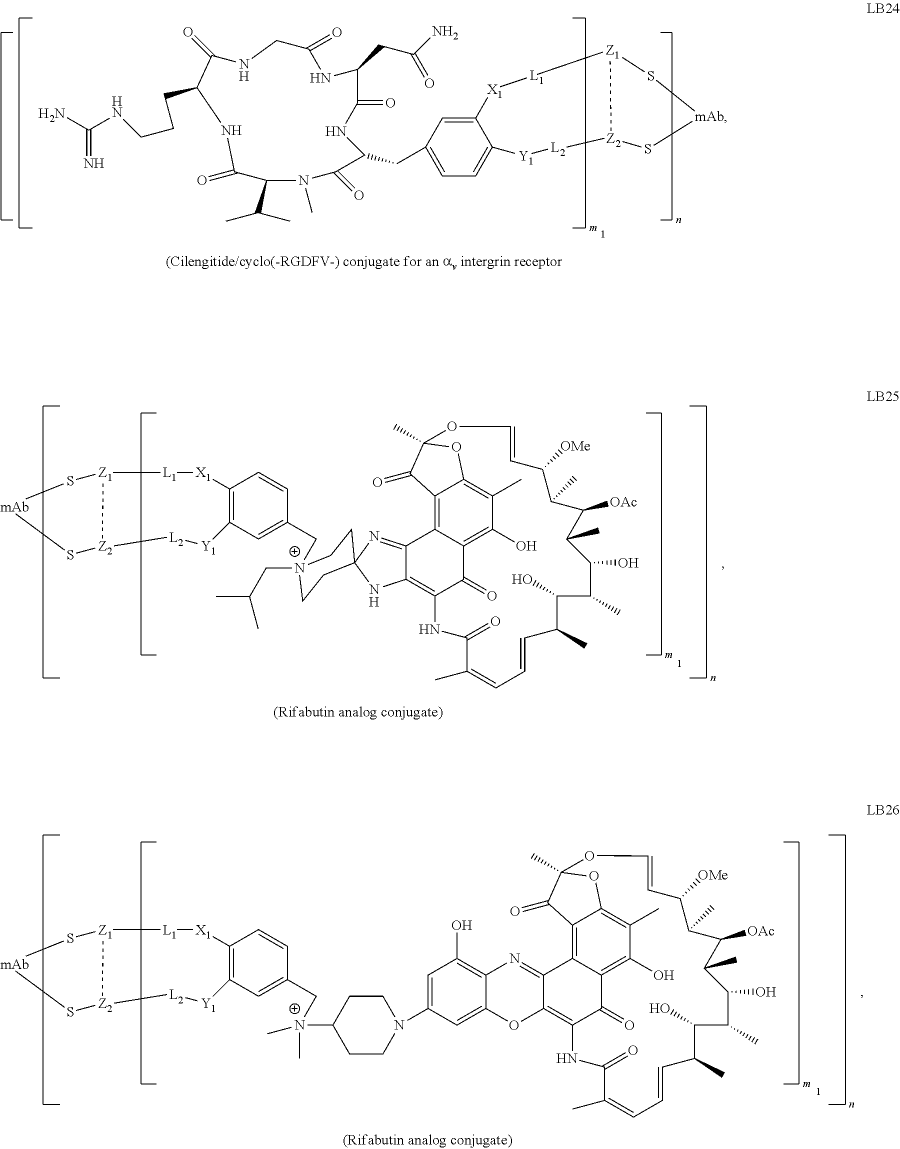 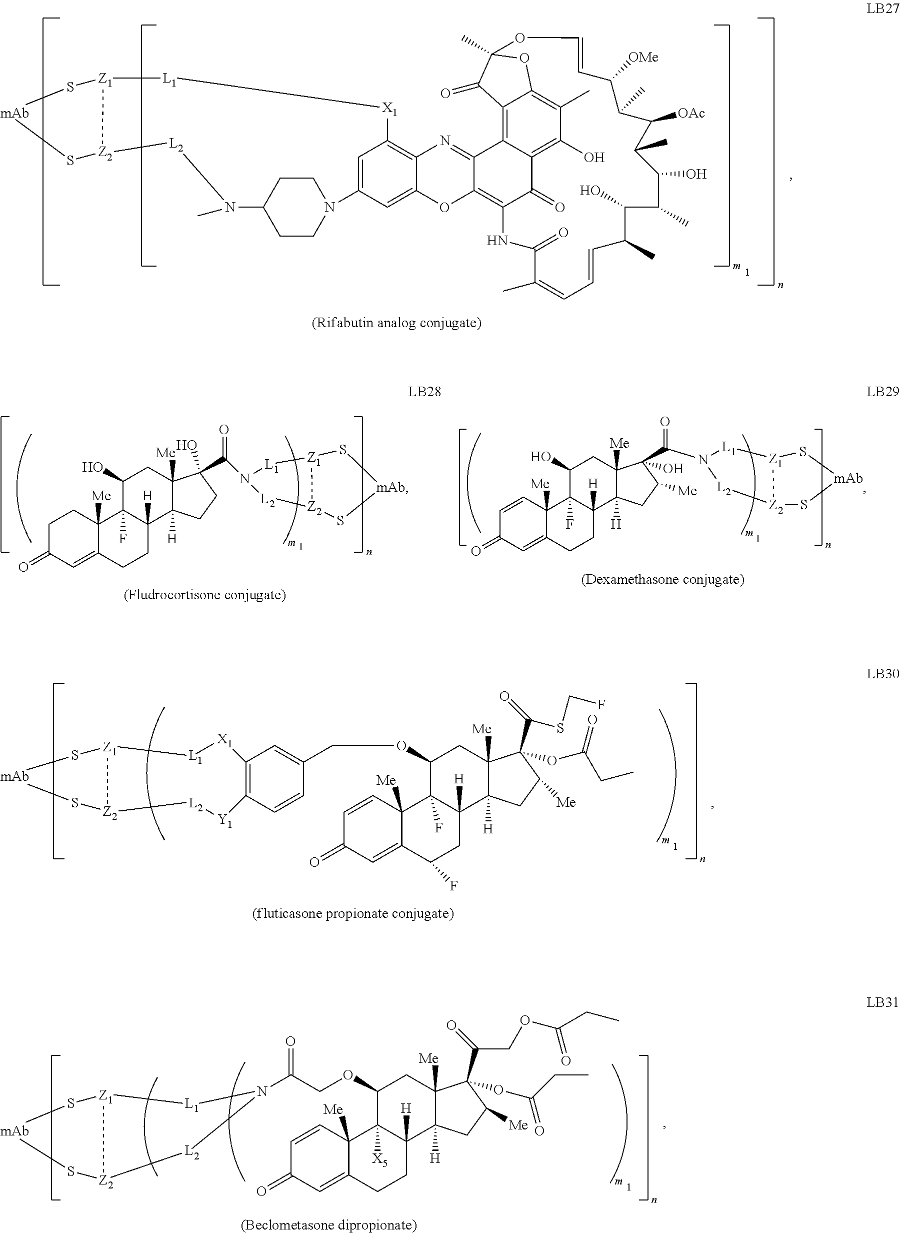 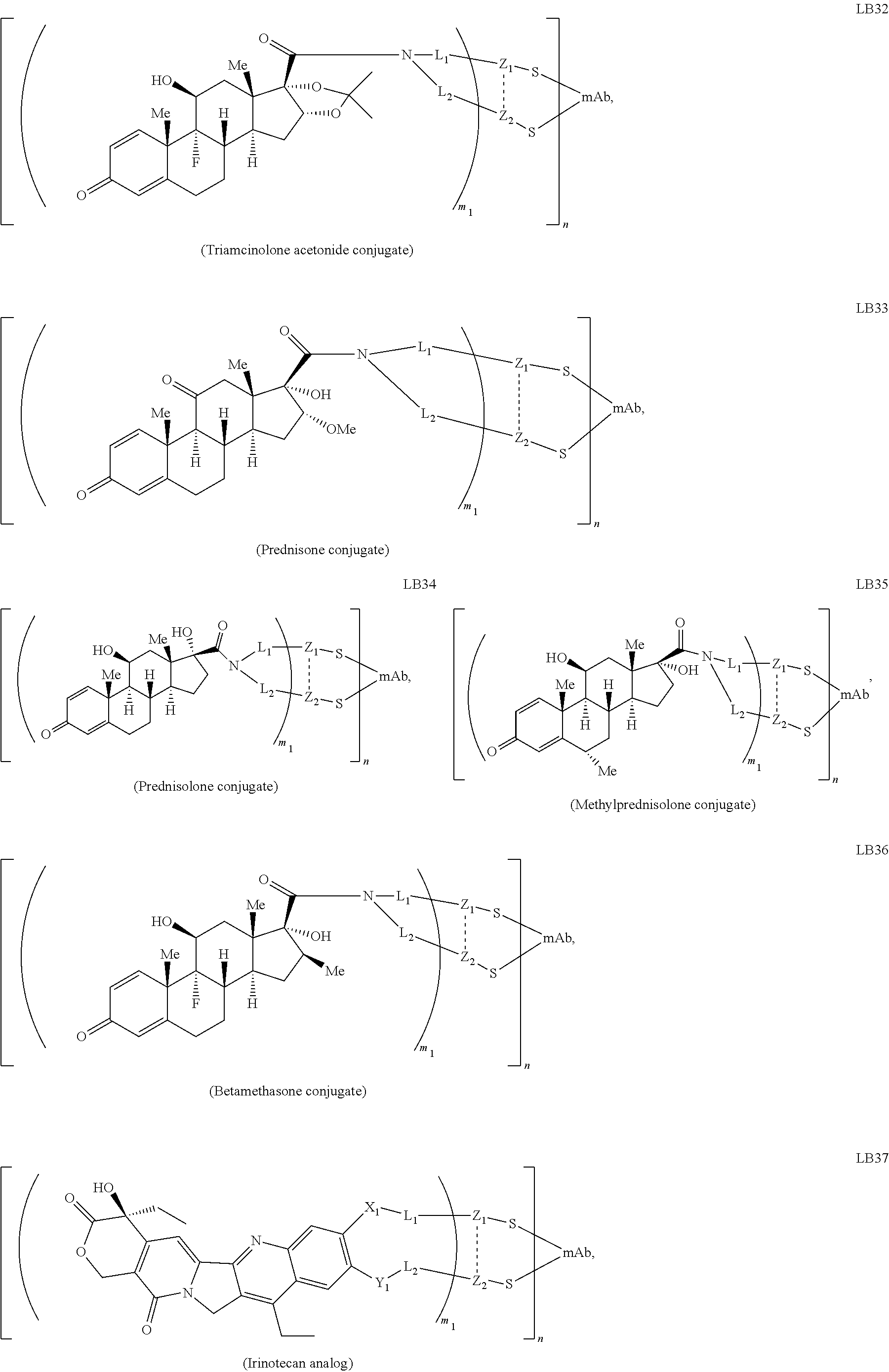 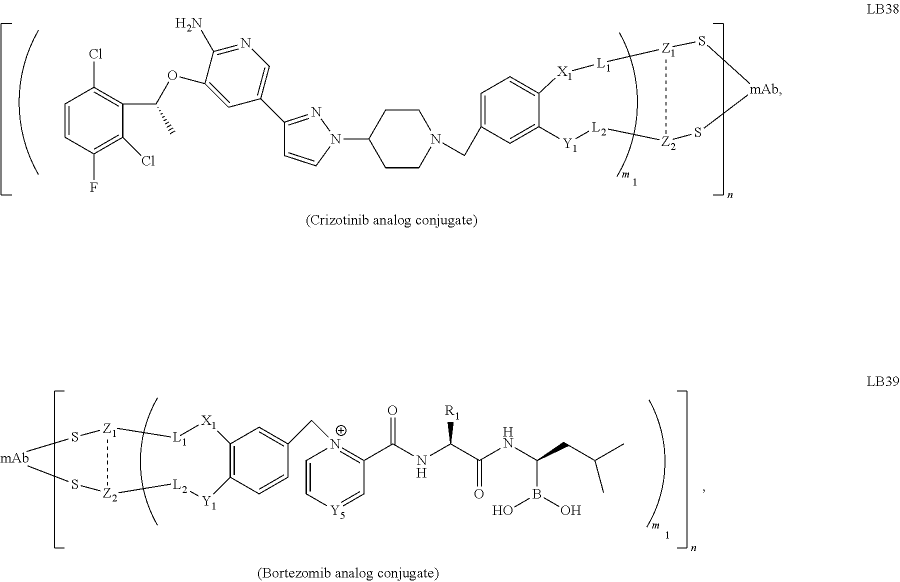
wherein Y₅, is N, CH, C(Cl), C(CH₃), or C(COOR₁); R₁ is H, C₁-C₆ Alkyl, or C₃-C₈ Ar, 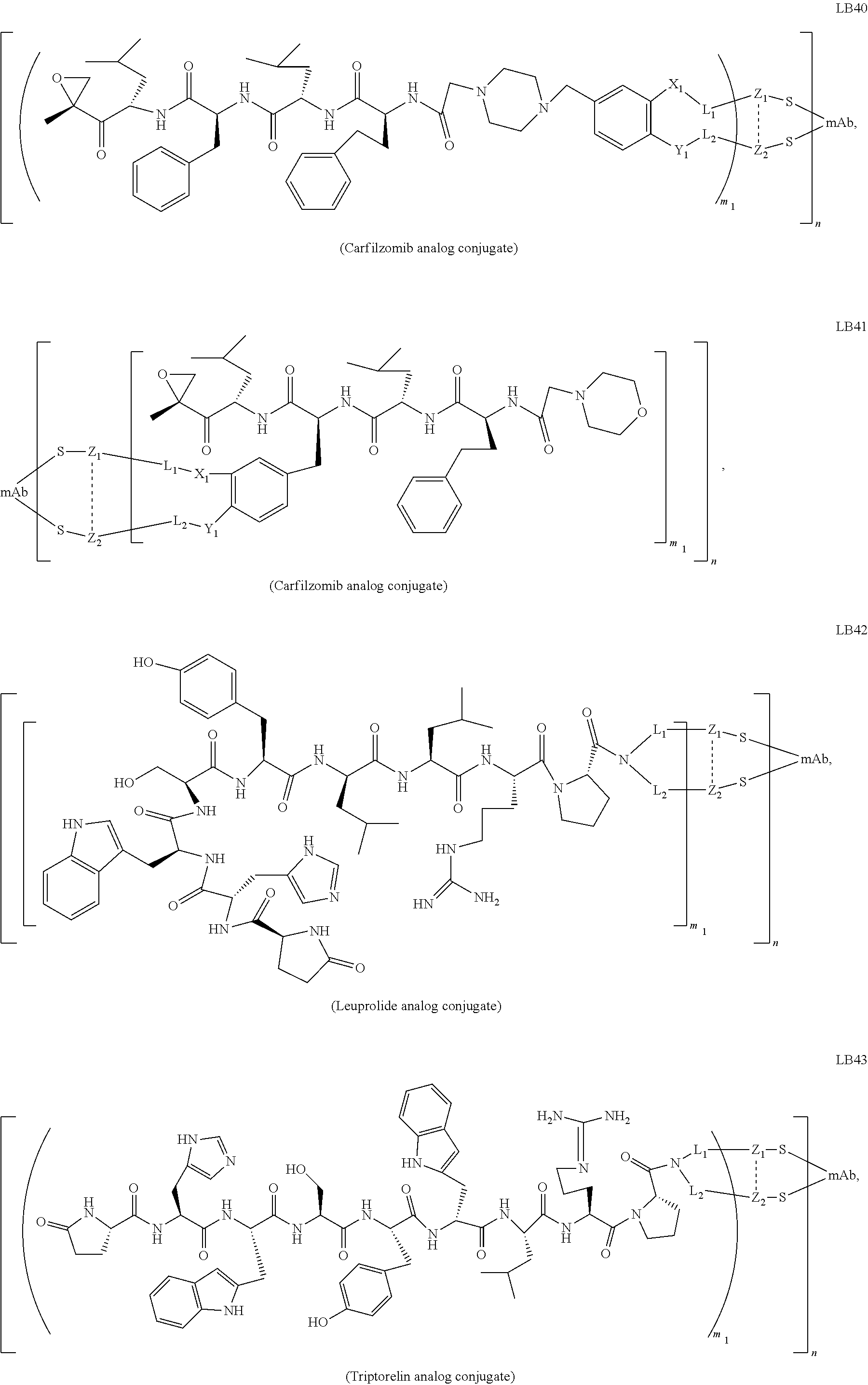 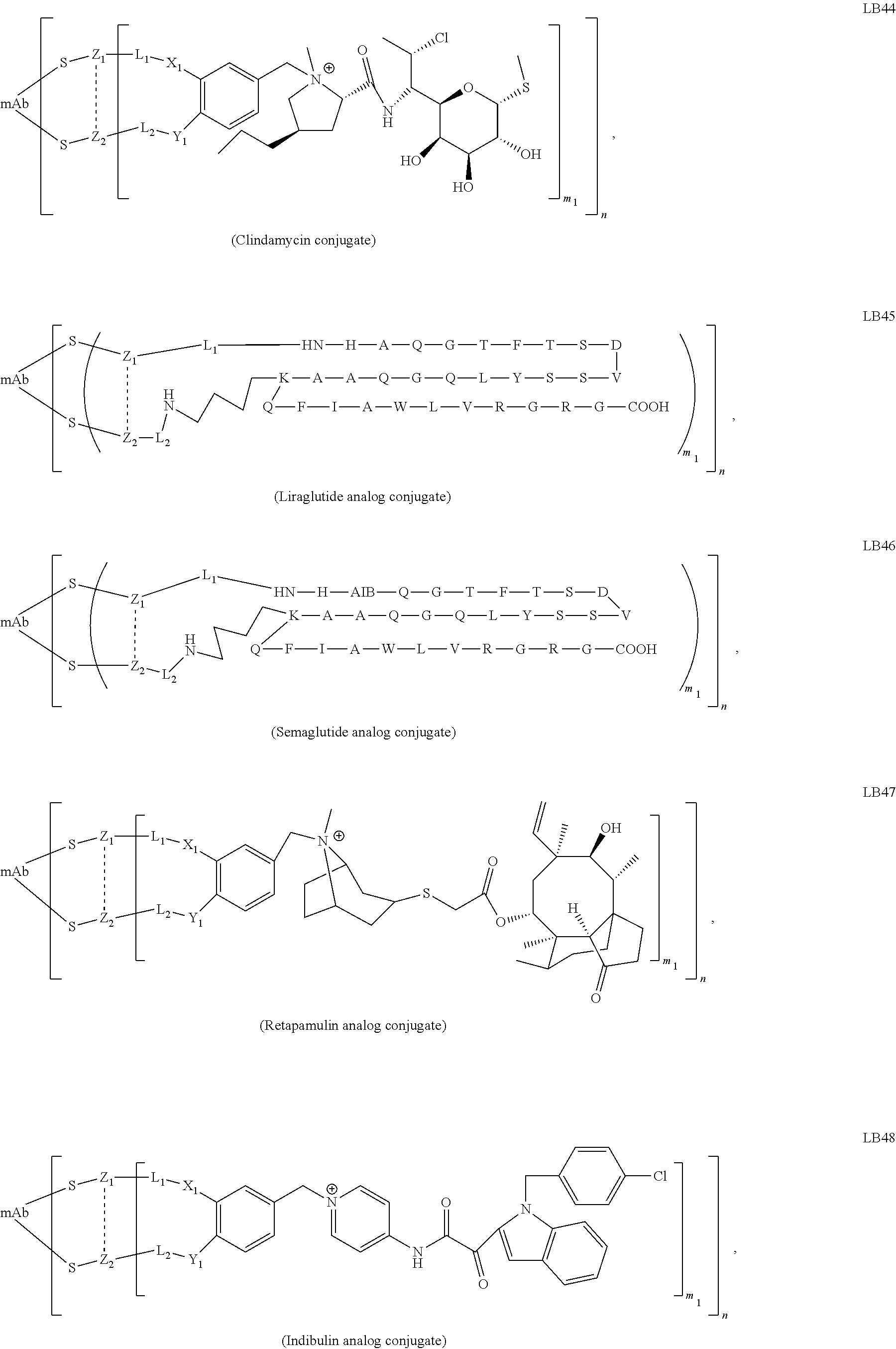 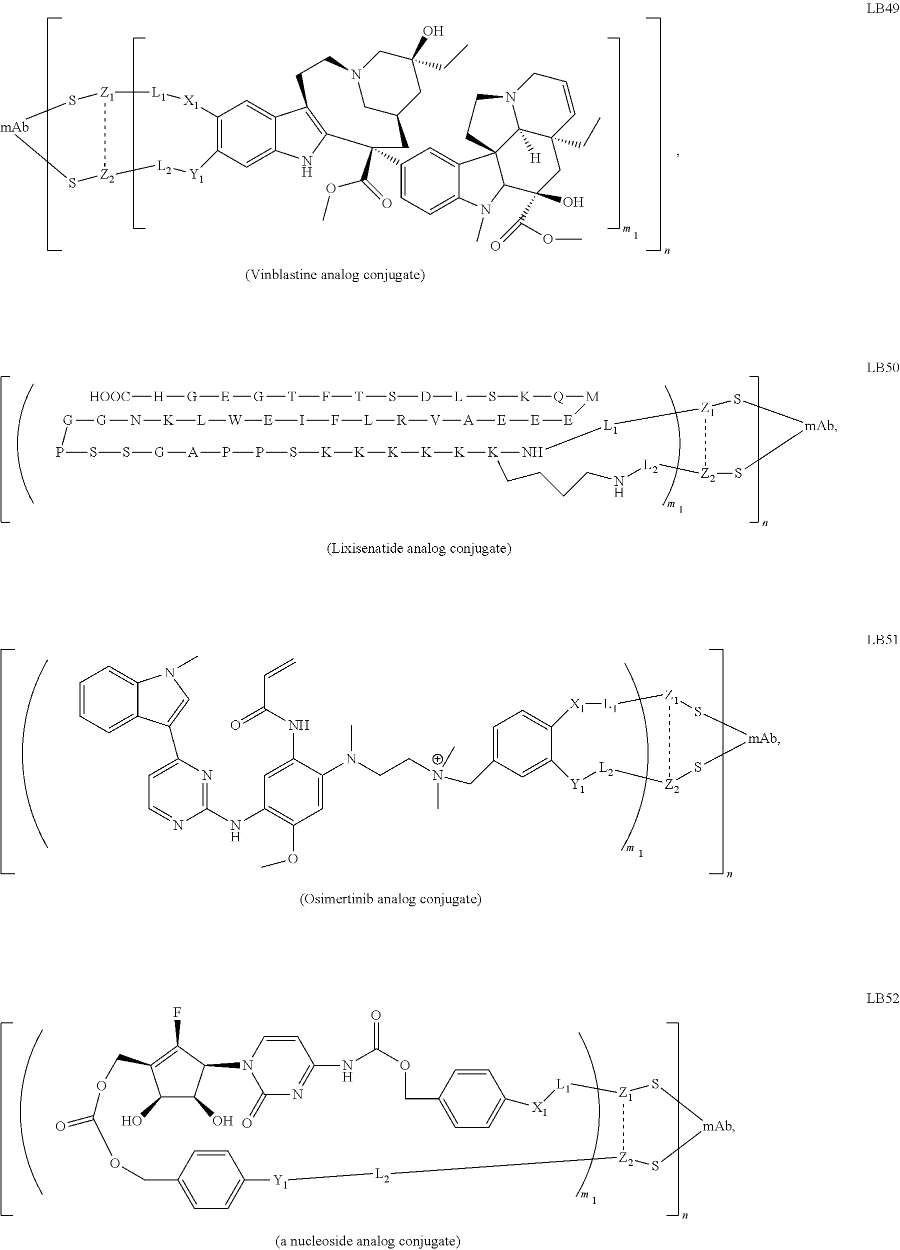 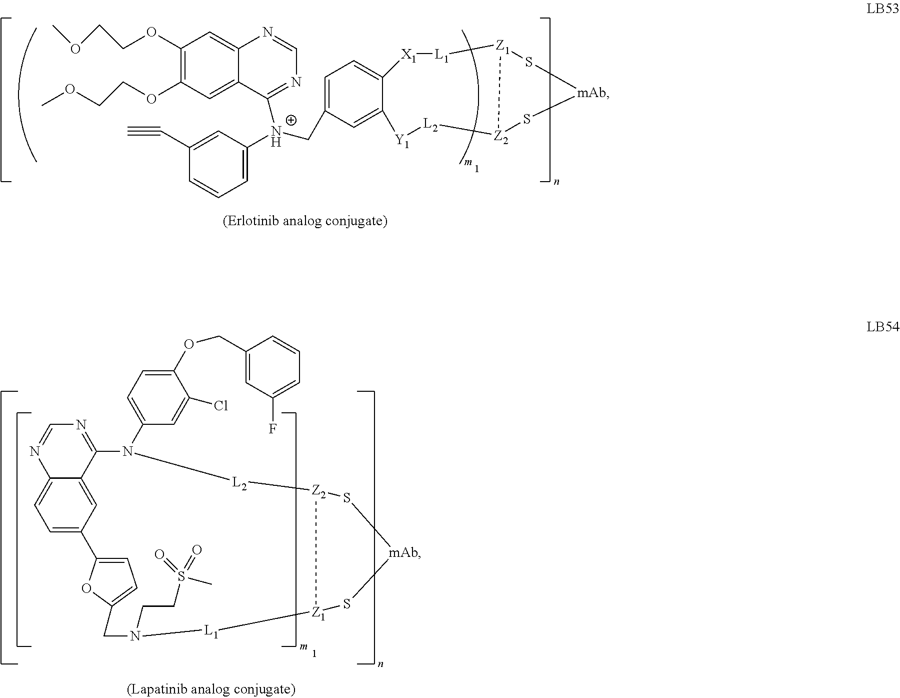
wherein “-----” is a single bond, a double bond, or absent; X₁, and Y₁ are independently O, NH, NHNH, NR₅, S, C(O)O, C(O)NH, OC(O)NH, OC(O)O, NHC(O)NH, NHC(O)S, OC(O)N(R₁), N(R₁)C(O)N(R₁), CH, C(O)NHNHC(O) or C(O)NR₁; mAb is antibody; n and m₁ are independently 1-20; X₃ is CH₂, O, NH, NHC(O), NHC(O)NH, C(O), OC(O), OC(O)(NR₃), R₁, NHR₁, NR₁, C(O)R₁ or absent; X₄ is H, CH₂, OH, O, C(O), C(O)NH, C(O)N(R₁), R₁, NHR₁, NR₁, C(O)R₁ or C(O)O; X₅ is H, CH₃, F, or Cl; M₁ and M₂ are independently H, Na, K, Ca, Mg, NH₄, or NR₁R₂R₃; R₆ is 5′-deoxyadenosyl, Me, OH, or CN; L₁, L₂, R₁, R₁′, R₂, Z₁, and Z₂, are defined the same as in claim 1.

31. The conjugate compound of claim 1, wherein conjugate compound has structure of SI-1 below: 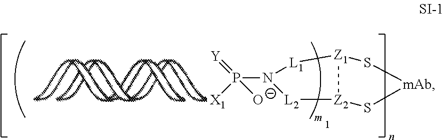
wherein mAb, m₁, n, X₁, L₁, L₂, Z₁, Z₂, “-----” are defined the same as in claim 1; [symbol] is single or double strands of DNA, RNA, mRNA, siRNA, miRNA, or piRNA; Y is O, S, NH or CH₂.

32. The conjugate compound according to claim 1, wherein the cell-binding molecule is an IgG antibody, monoclonal antibody, or an IgG antibody-like protein; and wherein Z1 and Z2 are connected to the cell-binding molecule via a pair of thiols generated through reduction of disulfide bonds of the cell-binding molecule between a light chain and heavy chain, upper disulfide bonds between two heavy chains and lower disulfide bonds between two heavy chains, the conjugate compound having following structure of ST1, ST2, ST3, ST4, ST5, or ST6: [symbol] [symbol] [symbol] [symbol] [symbol] [symbol]
wherein Z₁, Z₂, X, Y, L₁, L₂, “-----”, m₁, and cytotoxic molecule are defined the same as in claim 1.

33. The conjugate compound according to claim 32, wherein the cytotoxic molecules and m₁ at different conjugation sites of the cell-binding molecule are different when the cytotoxic molecules containing the same or different bis-linkers are conjugated to the cell-binding molecule sequentially, or when different cytotoxic molecules containing the same or different bis-linkers are added stepwisely in a conjugation reaction mixture containing a cell-binding molecule.

34. The conjugate compound according to claim 32, wherein the cytotoxic molecule is selected from tubulysins, maytansinoids, taxanoids (taxanes), CC-1065 analogs, daunorubicin and doxorubicin compounds, indolecarboxamide, benzodiazepine dimers, pyrrolobenzodiazepine (PBD) dimers, tomaymycin dimers, anthramycin dimers, indolinobenzodiazepines dimers, imidazobenzothiadiazepines dimers, oxazolidinobenzodiazepines dimers, calicheamicins and the enediyne antibiotics, actinomycin, amanitins, amatoxins, azaserines, bleomycins, epirubicin, eribulin, tamoxifen, idarubicin, dolastatins, auristatins (comprising monomethyl auristatin E, MMAE-, MMAF, auristatin PYE, auristatin TP, Auristatins 2-AQ, 6-AQ, EB (AEB), EFP (AEFP) and their analogs), duocarmycins, geldanamycins or other HSP90 inhibitors, centanamycin, methotrexates, thiotepa, vindesines, vincristines, hemiasterlins, nazumamides, microginins, radiosumins, streptonigtin, SN38 or other analogs or metabolites of camptothecin, alterobactins, microsclerodermins, theonellamides, esperamicins, PNU-159682, and their analogues or derivatives, pharmaceutically acceptable salts, acids, derivatives, hydrate or hydrated salt, a crystalline structure, an optical isomer, racemate, diastereomer or enantiomer of any of the above drugs; or the cytotoxic molecule is selected from:
(1), a chemotherapeutic agent selected from the group consisting of:
a). an alkylating agent: selected from the group consisting of nitrogen mustards: chlorambucil, chlomaphazine, cyclophosphamide, dacarbazine, estramustine, ifosfamide, mechlorethamine, mechlorethamine oxide hydrochloride, mannomustine, mitobronitol, melphalan, mitolactol, pipe-broman, novembichin, phenesterine, prednimustine, thiotepa, trofosfamide, uracil mustard; CC-1065 and adozelesin, carzelesin, bizelesin or their synthetic analogues; duocarmycin and its synthetic analogues, KW-2189, CBI-TMI, or CBI dimers; benzodiazepine dimers or pyrrolobenzodiazepine (PBD) dimers, tomaymycin dimers, indolinobenzodiazepine dimers, imidazobenzothiadiazepine dimers, or oxazolidinobenzodiazepine dimers; Nitrosoureas: comprising carmustine, lomustine, chlorozotocin, fotemustine, nimustine, ranimustine; Alkylsulphonates' comprising busulfan, treosulfan, improsulfan and piposulfan); Triazenes or dacarbazine; Platinum containing compounds: comprising, carboplatin, cisplatin, and oxaliplatin; aziridines, benzodopa, carboquone, meturedopa, or uredopa; ethylenimines and methylamelamines including altretamine, triethylenemelamine, trietylenephosphoramide, triethylenethiophosphoramide and trimethylolomelamine];
b). a plant alkaloid, selected from the group consisting of Vinca alkaloids: comprising vincristine, vinblastine, vindesine, vinorelbine, and navelbin; Taxoids; comprising paclitaxel, docetaxol and their analogs, Maytansinoids comprising DM1, DM2, DM3, DM4, DM5, DM6, DM7, maytansine, ansamitocins and their analogs, cryptophycins (including the group consisting of cryptophycin 1 and cryptophycin 8), epothilones, eleutherobin, discodermolide, bryostatins, dolostatins, auristatins, tubulysins, cephalostatins, pancratistatin, a sarcodictyin; spongistatin;
c). a DNA Topoisomerase inhibitor, selected from the groups of Epipodophyllins' comprising 9-aminocamptothecin, camptothecin, crisnatol, daunomycin, etoposide, etoposide phosphate, irinotecan, mitoxantrone, novantrone, retinoic acids (or retinols), teniposide, topotecan, 9-nitrocamptothecin or RFS 2000; and mitomycins and their analogs;
d). an antimetabolite: selected from the group consisting of {[Anti-folate, (DHFR inhibitors: comprising methotrexate, trimetrexate, denopterin, pteropterin, aminopterin (4-aminopteroic acid) or folic acid analogues); IMP dehydrogenase Inhibitors: (comprising mycophenolic acid, tiazofurin, ribavirin, EICAR); Ribonucleotide reductase Inhibitors: (comprising hydroxyurea, deferoxamine)]; [Pyrimidine analogs: Uracil analogs: (comprising ancitabine, azacitidine, 6-azauridine, capecitabine (Xeloda), carmofur, cytarabine, dideoxyuridine, doxifluridine, enocitabine, 5-Fluorouracil, floxuridine, ratitrexed (Tomudex)): Cytosine analogs: (comprising cytarabine, cytosine arabinoside, fludarabine), Purine analogs' (comprising azathioprine, fludarabine, mercaptopurine, thiamiprine, thioguanine)]: folic acid replenisher, frolinic acid};
e). a hormonal therapy selected from the group consisting of {Receptor antagonists: [Anti-estrogen: (comprising megestrol, raloxifene, tamoxifen); LHRH agonists: (comprising goscrclin, leuprolide acetate): Anti-androgens: (comprising bicalutamide, flutamide, calusterone, dromostanolone propionate, epitiostanol, goserelin, leuprolide, mepitiostane, nilutamide, testolactone, trilostane and other androgens inhibitors)]; Retinoids/Deltoids [Vitamin D3 analogs: (comprising CB 1003, EB 1089 KH 1060, cholecalciferol, ergocalciferol): Photodynamic therapies, (comprising verteporfin, phthalocyanine, photosensitizer Pc4, demethoxyhypocrellin A): Cytokines: (comprising interferon-alpha, interferon-gamma, tumor necrosis factor (TNFs), human proteins containing a TNF domain)]};
f). a kinase inhibitor, selected from the group consisting of BIBW 2992 (anti-EGFR/Erb2), imatinib, gefitinib, pegaptanib, sorafenib, dasatinib, sunitinib, erlotinib, nilotinib, lapatinib, axitinib, pazopanib, vandetanib, E7080 (anti-VEGFR2), mubritinib, ponatinib (AP24534), bafetinib (INNO-406), bosutinib (SKI-606), cabozantinib, vismodegib, iniparib, ruxolitinib, CYT387, axitinib, tivozanib, sorafenib, bevacizumab, cetuximab, Trastuzumab, Ranibizumab, Panitumumab, and ispinesib;
g). a poly (ADP-ribose) polymerase (PARP) inhibitor selected from the group consisting of olaparib, niraparib, iniparib, talazoparib, veliparib, CEP 9722 (Cephalon's), E7016 (Eisai's), BGB-290 (BeiGene's), and 3-aminobenzamide;
h). an antibiotic, selected from the group consisting of an enediyne antibiotic (selected from the group consisting of calicheamicin, calicheamicin γ1, δ1, α1 or β1; dynemicin, including dynemicin A and deoxydynemicin; esperamicin, kedarcidin, C-1027, maduropeptin, or neocarzinostatin chromophore and related chromoprotein enediyne antibiotic chromomophores), aclacinomycins, actinomycin, authramycin, azaserine, bleomycins, cactinomycin, carabicin, carminomycin, carzinophilin; chromomycins, dactinomycin, daunorubicin, detorubicin, 6-diazo-5-oxo-L-norleucine, doxorubicin, morpholino-doxorubicin, cyanomorpholino-doxorubicin, 2-pyrrolino-doxorubicin and deoxydoxorubicin, epirubicin, eribulin, esorubicin, idarubicin, marcellomycin, nitomycins, mycophenolic acid, nogalamycin, olivomycins, peplomycin, potfiromycin, puromycin, quelamycin, rodorubicin, streptonigrin, streptozocin, tuberculin, ubenimex, zinostatin, and zorubicin;
i). a polyketide (acetogenin), bullatacin and bullatacinone; gemcitabine, epoxomicins and -carfilzomib, bortezomib, thalidomide, lenalidomide, pomalidomide, tosedostat, zybrestat, PLX4032, STA-9090, Stimuvax, allovectin-7, Xegeva, Provenge, Yervoy, Isoprenylation inhibitors and Lovastatin, Dopaminergic neurotoxins and 1-methyl-phenylpyridinium ion, Cell cycle inhibitors (selected from staurosporine), Actinomycins (comprising Actinomycin D, dactinomycin), amanitins, Bleomycins (comprising bleomycin A2, bleomycin B2, peplomycin), Anthracyclines (comprising daunorubicin, doxorubicin (adriamycin), idarubicin, epirubicin, pirarubicin, zorubicin, mtoxantrone, MDR inhibitors or verapamil, Ca²⁺ ATPase inhibitors or thapsigargin, Histone deacetylase inhibitors ((comprising Vorinostat, Romidepsin, Panobinostat, Valproic acid, Mocetinostat (MGCD0103V Belinostat, PCI-24781, Entinostat, S8939, Resminostat, Givinostat, AR-42, CUDC-101, sulforaphane, Trichostatin A); Thapsigargin, Celecoxib, glitazones, epigallocatechin gallate, Disulfiram, Salinosporamide A; Anti-adrenals, selected from the group consisting of aminoglutethimide, mitotane, trilostane; aceglatone; aldophosphamide glycoside; aminolevulinic acid; amsacrine; arabinoside, bestrabucil; bisantrene; edatraxate; defofamine; demecolcine; diaziquone; eflornithine (DFMO), elfomithine; elliptinium acetate, etoglucid; gallium nitrate, gacytosine, hydroxyurea; ibandronate, lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; rhizoxin; sizofiran; spirogermanium; tenuazonic acid; triaziquone; 2, 2′,2″-trichlorotriethylamine; trichothecenes (including the group consisting of T-2 toxin, verrucarin A, roridin A and anguidine); urethane, siRNA, antisense drugs;
(2). an anti-autoimmune disease agent: cyclosporine, cyclosporine A, aminocaproic acid, azathioprine, bromocriptine, chlorambucil, chloroquine, cyclophosphamide, corticosteroids (including the group consisting of amcinonide, betamethasone, budesonide, hydrocortisone, flunisolide, fluticasone propionate, fluocortolone danazol, dexamethasone, Triamcinolone acetonide, beclometasone dipropionate), DHEA, enanercept, hydroxychloroquine, infliximab, meloxicam, methotrexate mofetil, mycophenylate, prednisone, sirolimus, tacrolimus;
(3) an anti-infectious disease agents comprising:
a). aminoglycosides: amikacin, astromicin, gentamicin (netilmicin, sisomicin, isepamicin), hygromycin B, kanamycin (amikacin, arbekacin, bekanamycin, dibekacin, tobramycin), neomycin (framycetin, paromomycin, ribostamycin), netilmicin, spectinomycin, streptomycin, tobramycin, verdamicin;
b). amphenicols: azidamfenicol, chloramphenicol, florfenicol, thiamphenicol;
c). ansamycins: geldanamycin, herbimycin;
d). carbapenems: biapenem, doripenem, ertapenem, imipenem/cilastatin, meropenem, panipenem;
e). cephems: carbacephem (loracarbef), cefacetrile, cefaclor, cefradine, cefadroxil, cefalonium, cefaloridine, cefalotin or cefalothin, cefalexin, cefaloglycin, cefamandole, cefapirin, cefatrizine, cefazaflur, cefazedone, cefazolin, cefbuperazone, cefcapene, cefdaloxime, cefepime, cefminox, cefoxitin, cefprozil, cefroxadine, ceftezole, cefuroxime, cefixime, cefdinir cefditoren, cefepime, cefetamet, cefmenoxime, cefodizime, cefonicid, cefoperazone, ceforanide, cefotaxime, cefotiam, cefozopran, cephalexin, cefpimizole, cefpiramide, cefpirome, cefpodoxime, cefprozil, cefquinome, cefsulodin, ceftazidime, cefteram, ceftibuten, ceftiolene, ceftizoxime, ceftobiprole, ceftriaxone, cefuroxime, cefuzonam, cephamycin (cefoxitin, cefotetan, cefmetazole), oxacephem (flomoxef, latamoxef);
f). glycopeptides: bleomycin, vancomycin (oritavancin, telavancin), teicoplanin (dalbavancin), ramoplanin;
g). glycylcyclines: tigecycline;
h). β-lactamase inhibitors: penam (sulbactam, tazobactam), clavam (clavulanic acid);
i). lincosamides: clindamycin, lincomycin;
j). lipopeptides: daptomycin, A54145, calcium-dependent antibiotics (CDA);
k). macrolides: azithromycin, cethromycin, clarithromycin, dirithromycin, erythromycin, flurithromycin, josamycin, ketolide (telithromycin, cethromycin), midecamycin, miocamycin, oleandomycin, rifamycins (rifampicin, rifampin, rifabutin, rifapentine), rokitamycin, roxithromycin, spectinomycin, spiramycin, tacrolimus (FK506), troleandomycin, telithromycin;
l). monobactams: aztreonam, tigemonam;
m). oxazolidinones, linezolid;
n). penicillins: amoxicillin, ampicillin, pivampicillin, hetacillin, bacampicillin, metampicillin, talampicillin, azidocillin, azlocillin, benzyl penicillin, benzathine benzylpenicillin, benzathine phenoxymethylpenicillin, clometocillin, procaine benzylpenicillin, carbenicillin (carindacillin), cloxacillin, dicloxacillin, epicillin, flucloxacillin, mecillinam (pivmecillinam), mezlocillin, meticillin, nafcillin, oxacillin, penamecillin, penicillin, pheneticillin, phenoxymethylpenicillin, piperacillin, propicillin, sulbenicillin, temocillin, ticarcillin;
o). polypeptides: bacitracin, colistin, polymyxin B;
p). quinolones: alatrofloxacin, balofloxacin, ciprofloxacin, clinafloxacin, danofloxacin, difloxacin, enoxacin, enrofloxacin, floxin, garenoxacin, gatifloxacin, gemifloxacin, grepafloxacin, kano trovafloxacin, levofloxacin, lomefloxacin, marbofloxacin, moxifloxacin, nadifloxacin, norfloxacin, orbifloxacin, ofloxacin, pefloxacin, trovafloxacin, grepafloxacin, sitafloxacin, sparfloxacin, temafloxacin, tosufloxacin, trovafloxacin;
q). streptogramins: pristinamycin, quinupristin/dalfopristin;
r). sulfonamides: mafenide, prontosil, sulfacetamide, sulfamethizole, sulfanilimide, sulfasalazine, sulfisoxazole, trimethoprim, trimethoprim-sulfamethoxazole (co-trimoxazole);
s). steroid antibacterials: fusidic acid;
t). tetracyclines, doxycycline, chlortetracycline, clomocycline, demeclocycline, lymecycline, meclocycline, metacycline, minocycline, oxytetracycline, penimepicycline, rolitetracycline, tetracycline, glycylcyclines (including tigecycline);
u). antibiotics: annonacin, arsphenamine, bactoprenol inhibitors (Bacitracin), DADAL/AR inhibitors (cycloserine), dictyostatin, discodermolide, eleutherobin, epothilone, ethambutol, etoposide, faropenem, fusidic acid, furazolidone, isoniazid, laulimalide, metronidazole, mupirocin, mycolactone, NAM synthesis inhibitors (fosfomycin), nitrofurantoin, paclitaxel, platensimycin, pyrazinamide, quinupristin/dalfopristin, rifampicin (rifampin), tazobactam tinidazole, uvaricin;
(4). anti-viral drugs comprising:
a). entry/fusion inhibitors: aplaviroc, maraviroc, vicriviroc, gp41 (enfuvirtide), PRO 140, CD-4 (ibalizumab);
b). integrate inhibitors: raltegravir, elvitegravir, globoidnan A;
c). maturation inhibitors: bevirimat, vivecon;
d). neuraminidase inhibitors: oseltamivir, zanamivir, peramivir;
e). nucleosides & nucleotides: abacavir, aciclovir, adefovir, amdoxovir, apricitabine, brivudine, cidofovir, clevudine, dexelvucitabine, didanosine (ddI), elvucitabine, emtricitabine (FTC), entecavir, famciclovir, fluorouracil (5-FU), 3′-fluoro-substituted 2′,3′-dideoxynucleoside analogues (including the group consisting of 3′-fluoro-2′,3′-dideoxythymidine (FLT) and 3′-fluoro-2′,3′-dideoxyguanosine (FLG), fomivirsen, ganciclovir, idoxuridine, lamivudine (3TC), 1-nucleosides (including the group consisting of β-1-thymidine and β-1-2′-deoxycytidine), penciclovir, racivir, ribavirin, stampidine, stavudine (d4T), taribavirin (viramidine), telbivudine, tenofovir, trifluridine valaciclovir, valganciclovir, zalcitabine (ddC), zidovudine (AZT);
f). non-nucleosides: amantadine, ateviridine, capravirine, diarylpyrimidines (etravirine, rilpivirine), delavirdine, docosanol, emivirine, efavirenz, foscarnet (phosphonoformic acid), imiquimod, interferon alfa, loviride, lodenosine, methisazone, nevirapine, NOV-205, peginterferon alfa, podophyllotoxin, rifampicin, rimantadine, resiquimod (R-848), tromantadine;
g). protease inhibitors: amprenavir, atazanavir, boceprevir, darunavir, fosamprenavir, indinavir, lopinavir, nelfinavir, pleconaril, ritonavir, saquinavir, telaprevir (VX-950), tipranavir;
h). anti-virus drugs: abzyme, arbidol, calanolide a, ceragenin, cyanovirin-n, diarylpyrimidines, epigallocatechin gallate (EGCG), foscarnet, griffithsin, taribavirin (viramidine), hydroxyurea, KP-1461, miltefosine, pleconaril, portmanteau inhibitors, ribavirin, seliciclib;
(5). a radioisotope selected from the group consisting of (radionuclides) ³H, ¹¹C, ¹⁴C, ¹⁸F, ³²P, ³⁵S, ⁶⁴Cu, ⁶⁸Ga, ⁸⁶Y, ⁹⁹Tc, ¹¹¹In, ¹²³I, ¹²⁴I, ¹²⁵I, ¹³¹I, ¹³³Xe, ¹⁷⁷Lu, ²¹¹At, and ²¹³Bi;
(6). a chromophore molecule, which is capable of absorbing UV light, fluorescent light, IR light, phores, melanophores, cyanophores, fluorophore molecules which are fluorescent chemical compounds reemitting light upon light, visual phototransduction molecules, photophore molecules, luminescence molecules, luciferin compounds; Non-protein organic fluorophores selected from: Xanthene derivatives (comprising fluorescein, rhodamine, Oregon green, eosin, and Texas red); Cyanine derivatives (comprising cyanine, indocarbocyanine, oxacarbocyanine, thiacarbocyanine, and merocyanine); Squaraine derivatives and ring-substituted squaraines, including Seta, SeTau, and Square rives: Naphthalene derivatives (comprising dansyl and prodan derivatives): Coumarin derivatives: Oxadiazole derivatives (comprising pyridyloxazole, nitrobenzoxadiazole and benzoxadiazole); Anthracene derivatives (comprising anthraquinones, including DRAQ5, DRAQ7 and CYTRAK Orange); Pyrene derivatives (cascade blue)l Oxazine derivatives (comprising Nile red, Nile blue, cresyl violet, oxazine 170): Acridine derivatives (comprising proflavin, acridine orange, acridine yellow); Arylmethine derivatives (comprising auramine, crystal violet, malachite green); Tetrapyrrole derivatives (comprising porphin, phthalocyanine, bilirubin); analogs and derivatives of fluorophore compounds comprising CF dye, DRAQ and CyTRAK probes, BODIPY, Alexa Fluor, DyLight Fluor, Atto and Tracy, FluoProbes, Abberior Dyes, DY and MegaStokes Dyes, Sulfo Cy dyes, HiLyte Fluor, Seta, SeTau and Square Dyes, Quasar and Cal Fluor dyes, SureLight Dyes (APC RPEPerCP, Phycobilisomes), APC, APCXL, RPE, BPE, Allophycocyanin (APC), Aminocoumarin, APC-Cy7 conjugates, BODIPY-FL, Cascade Blue, Cy2, Cy3, Cy3.5, Cy3B, Cy5, Cy5.5, Cy7, Fluorescein, FluorX, Hydroxycoumarin, Lissamine Rhodamine B, Lucifer yellow, Methoxycoumarin, NBD, Pacific Blue, Pacific Orange, PE-Cy5 conjugates, PE-Cy7 conjugates, PerCP, R-Phycoerythrin(PE), Red 613, Seta-555-Azide, Seta-555-DBCO, Seta-S55-NHS, Seta-580-NHS, Seta-680-NHS, Seta-780-NHS, Seta-APC-780, Seta-PerCP-680, Seta-R-PE-670, SeTau-380-NHS, SeTau-405-Maleimide, SeTau-405-NHS, SeTau-423-NHS, SeTau-647-NHS, Texas Red, TRITC, TruRed, X-Rhodamine, 7-AAD (7-aminoactinomycin D, CG-selective), Acridine Orange, Chromomycin A3, CyTRAK Orange (red excitation dark), DAPI, DRAQ5, DRAQ7, Ethidium Bromide, Hoechst33258, Hoechst33342, LPS 751, Mithramycin, Propidium Iodide (PI), SYTOX Blue, SYTOX Green, SYTOX Prance, Thiazole Orange, TO-PRO: Cyanine Monomer, TOTO-1, TO-PRO-1, TOTO-3, TO-PRO-3, YOSeta-1, YOYO-1, a fluorophore compound comprising DCFH (2′7′-Dichorodihydro-fluorescein, oxidized form), DHR (Dihydrorhodamine 123, oxidized form, light catalyzes oxidation), Fluo-3 (AM ester, pH>6), Fluo-4 (AM ester, pH 7.2), Indo-1 (AM ester, low/high calcium (Ca2+), SNARF(pH 6/9), Allophycocyanin(APC), AmCyan1 (tetramer, Clontech), AsRed2 (tetramer, Clontech), Azami Green (monomer), Azurite, B-phycoerythrin (BPE) Cerulean, CyPet, DsRed monomer (Clontech), DsRed2 (“RFP”), EBFP, EBFP2, ECFP, EGFP (weak dimer), Emerald (weak dimer), EYFP (weak dimer), GFP (S65A mutation), GFP (S65C mutation), GFP (S65L mutation), GFP (S65T mutation), GFP (Y66F mutation), GFP (Y66H mutation), GFP (Y66W mutation), GFPuv, HcRed1, J-Red, Katusha, Kusabira Orange (monomer, MBL), mCFP, mCherry, mCitrine, Midoriishi Cyan (dimer, MBL), mKate (TagFP635, monomer), mKeima-Red (monomer), mKO, mOrange, mPlum, mRaspberry, mRFP1 (monomer), mStrawberry, mTFP1, mTurquoise2, P3 (phycobilisome complex), Peridinin Chlorophyll (PerCP), R-phycoerythrin (RPE), T-Sapphire, TagCFP (dimer), TagGFP (dimer), TagRFP (dimer), TagYFP (dimer), tdTomato (tandem dimer), Topaz, TurboFP602 (dimer), TurboFP635 (dimer), TurboGFP (dimer), TurboRFP (dimer), TurboYFP (dimer), Venus, Wild Type GFP, YPet, ZsGreen1 (tetramer), ZsYellow1 (tetramer) and their derivatives;
(7). cell-binding ligands or receptor agonists: Folate derivatives: Glutamic acid urea derivatives Somatostatin and its analogs (selected from the group consisting of octreotide (Sandostatin) and lanreotide (Somatuline)): Aromatic sulfonamides: Pituitary adenylate cyclase activating peptides (PACAP) (PAC1), Vasoactive intestinal peptides (VIP/PACAP) (VPAC1, VPAC2), Melanocyte-stimulating hormones (α-MSH): Cholecystokinins (CCK)/gastrin receptor agonists: Bombesins (selected from the group consisting of Pyr-Gln-Arg-Leu-Gly-Asn-Gln-Trp-Ala-Val-Gly-His-Leu-Met-NH₂/gastrin-releasing peptide (GRP): Neurotensin receptor ligands (NTR1, NTR2, NTR3); Substance P (NK1 receptor) ligands; Neuropeptide Y (Y1-Y6); Homing Peptides include RGD (Arg-Gly-Asp), NGR (Asn-Gly-Arg), dimeric and multimeric cyclic RGD peptides (selected from cRGDfV), TAASGVRSMH and LTLRWVGLMS (Chondroitin sulfate proteoglycan NG2 receptor ligands) and F3 peptides: Cell Penetrating Peptides (CPPs); Peptide Hormones, selected from the group consisting of luteinizing hormone-releasing hormone (LHRH) agonists and antagonists, and gonadotropin-releasing hormone (GnRH) agonist, acts by targeting follicle stimulating hormone (FSH) and luteinising hormone (LH), as well as testosterone production, selected from the group consisting of buserelin (Pyr-His-Trp-Ser-Tyr-D-Ser(OtBu)-Leu-Arg-Pro-NHEt), Gonadorelin (Pyr-His-Trp-Ser-Tyr-Gly-Leu-Arg-Pro-Gly-NH₂), Goserelin (Pyr-His-Trp-Ser-Tyr-D-Ser(OtBu)-Leu-Arg-Pro-AzGly-NH₂), Histrelin (Pyr-His-Trp-Ser-Tyr-D-His(N-benzyl)-Leu-Arg-Pro-NHEt), leuprolide (Pyr-His-Trp-Ser-Tyr-D-Leu-Leu-Arg-Pro-NHEt), Nafarelin (Pyr-His-Trp-Ser-Tyr-2Nal-Leu-Arg-Pro-Gly-NH₂), Triptorelin (Pyr-His-Trp-Ser-Tyr-D-Trp-Leu-Arg-Pro-Gly-NH₂), Nafarelin, Deslorelin, Abarelix (Ac-D-2Nal-D-4-chloroPhe-D-3-(3-pyridyl)Ala-Ser-(N-Me)Tyr-D-Asn-Leu-isopropylLys-Pro-DAla-NH₂), Cetrorelix (Ac-D-2Nal-D-4-chloroPhe-D-3-(3-pyridyl)Ala-Ser-Tyr-D-Cit-Leu-Arg-Pro-D-Ala-NH₂), Degarelix (Ac-D-2Nal-D-4-chloroPhe-D-3-(3-pyridyl)Ala-Ser-4-aminoPhe(L-hydroorotyl)-D-4-aminoPhe(carba-moyl)-Leu-isopropylLys-Pro-D-Ala-NH₂), and Ganirelix (Ac-D-2Nal-D-4-chloroPhe-D-3-(3-pyridyl)Ala-Ser-Tyr-D-(N9, N10-diethyl)-homoArg-Leu-(N9, N10-diethyl)-homoArg-Pro-D-Ala-NH₂): Pattern Recognition Receptor (PRRs), selected from the group consisting of Toll-like receptors' (TLRs) ligands, C-type lectins and Nodlike Receptors' (NLRs) ligands: Calcitonin receptor agonists; integrin receptors' and their receptor subtypes' (selected from the group consisting of α_{V}β₁, α_{V}β₃, α_{V}β₅, α_{V}β₆, α₆β₄, α₇β₁, α₁β₂, α_{IIb}β₃) agonists (selected from the group consisting of GRGDSPK, cyclo(RGdfV) (L1) and its derives [cyclo(-N(Me)R-GDfV), cyclo(R-Sar-DfV), cyclo(RG-N(Me)D-fV), cyclo(RGD-N(Me)f-V), cyclo(RGDf-N(Me)V-)(Cilengitide)], Nanobody (a derivative of VHH (camelid Ig)); Domain antibodies (dAb, a derivative of VH or VL domain): Bispecific T cell Engager (BiTE, a bispecific diabody); Dual Affinity ReTargeting (DART, a bispecific diabody), Tetravalent tandem antibodies (TandAb, a dimerized bispecific diabody): Anticalin (a derivative of Lipocalins); Adnectins (10th FN3 (Fibronectin)): Designed Ankyrin Repeat, Proteins (DARPins); Avimers: EGF receptors and VEGF receptors' agonists;
(8). pharmaceutically acceptable salts, acids, derivatives, hydrate or hydrated salt; or a crystalline structure; or an optical isomer, racemate, diastereomer or enantiomer of any of the above drugs.

35. The compound of Formula (II) according to claim 2, having one of following structures:

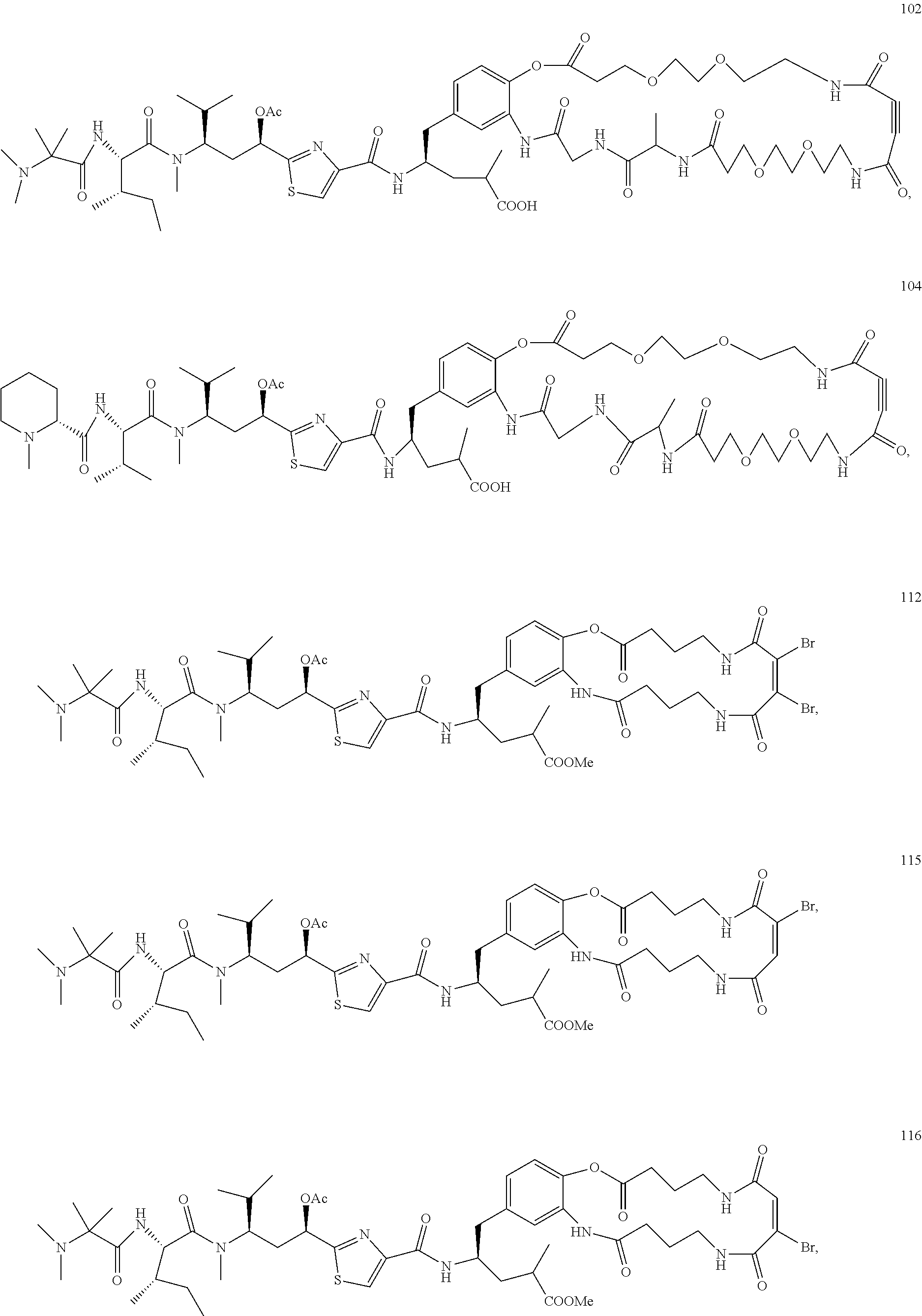

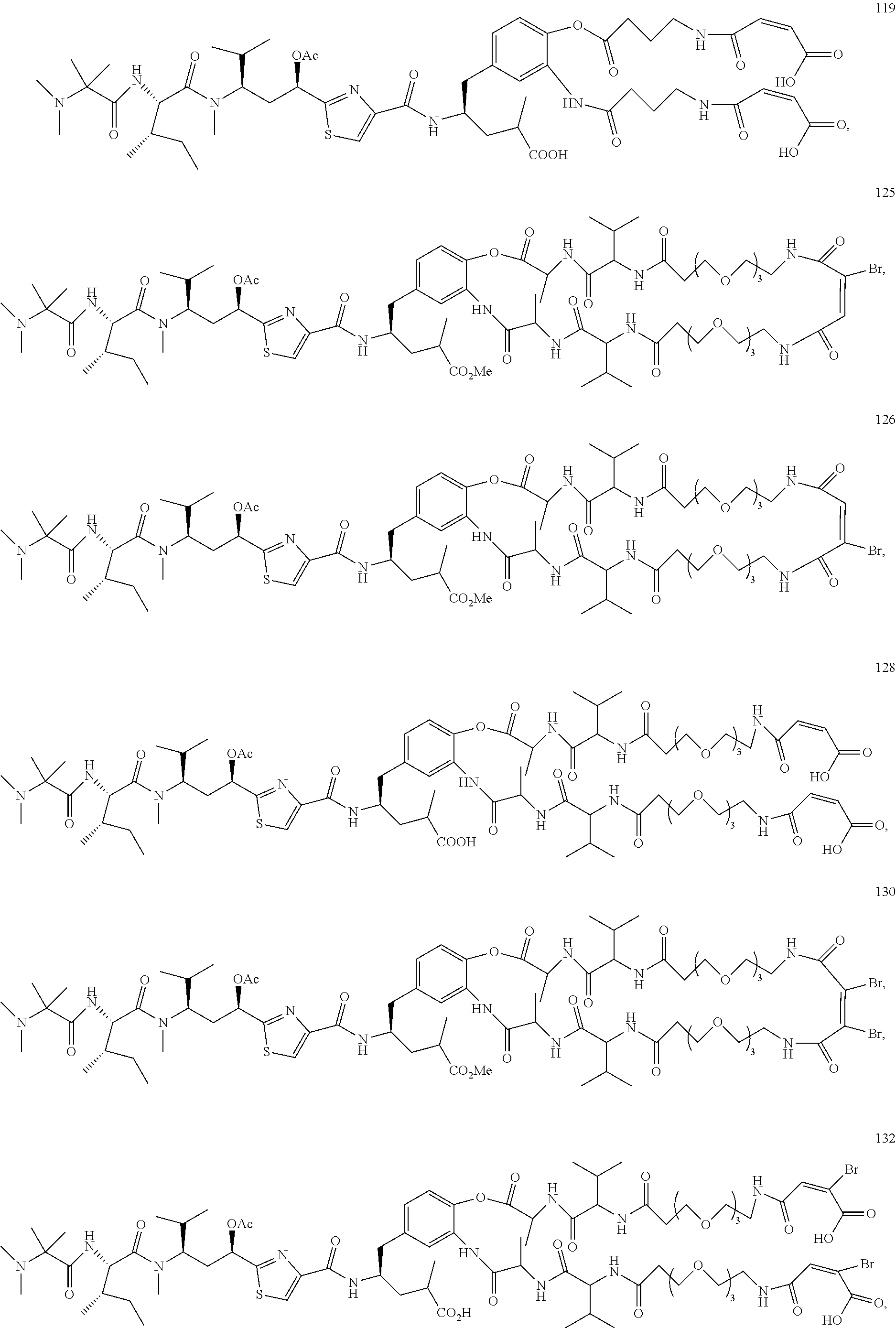

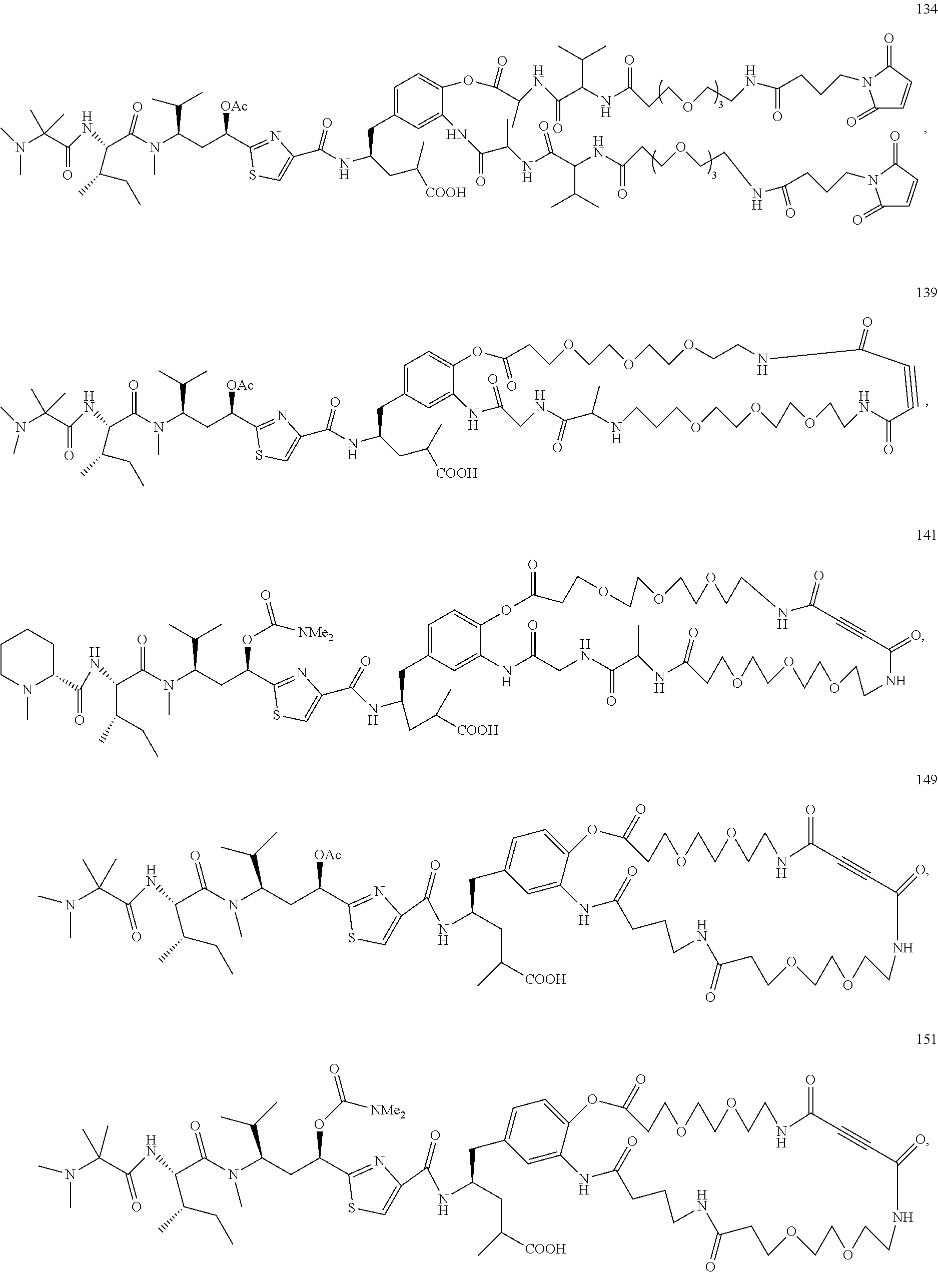

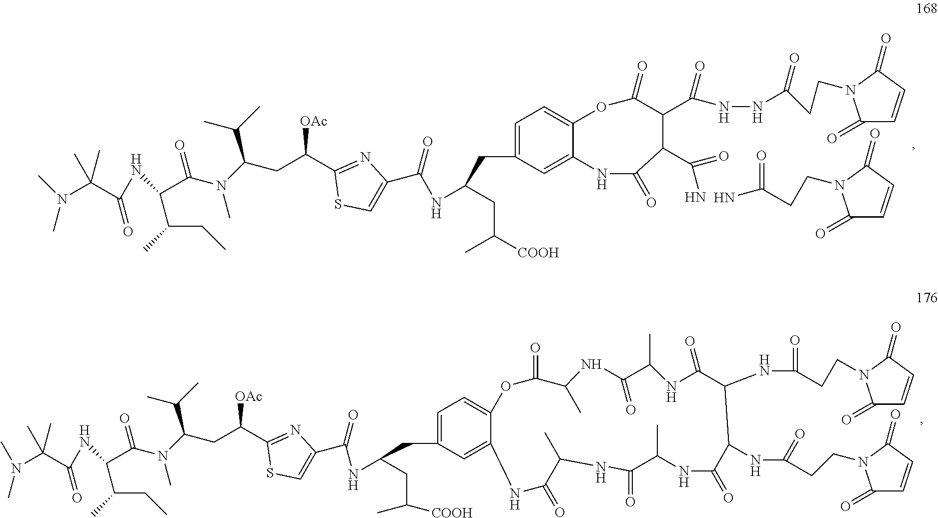

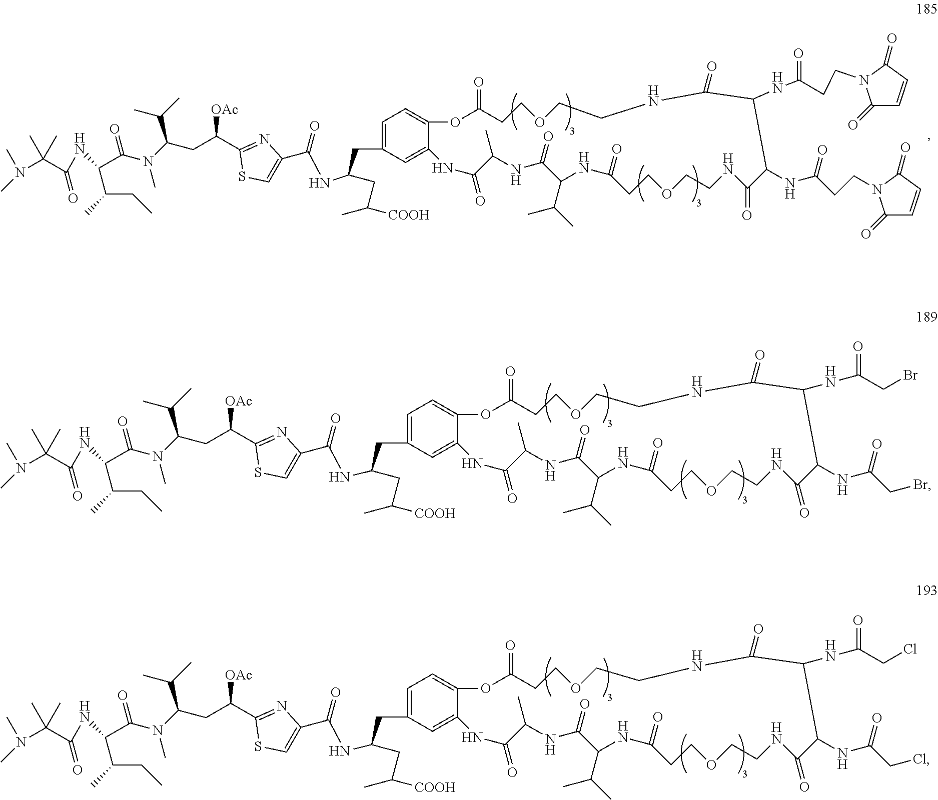

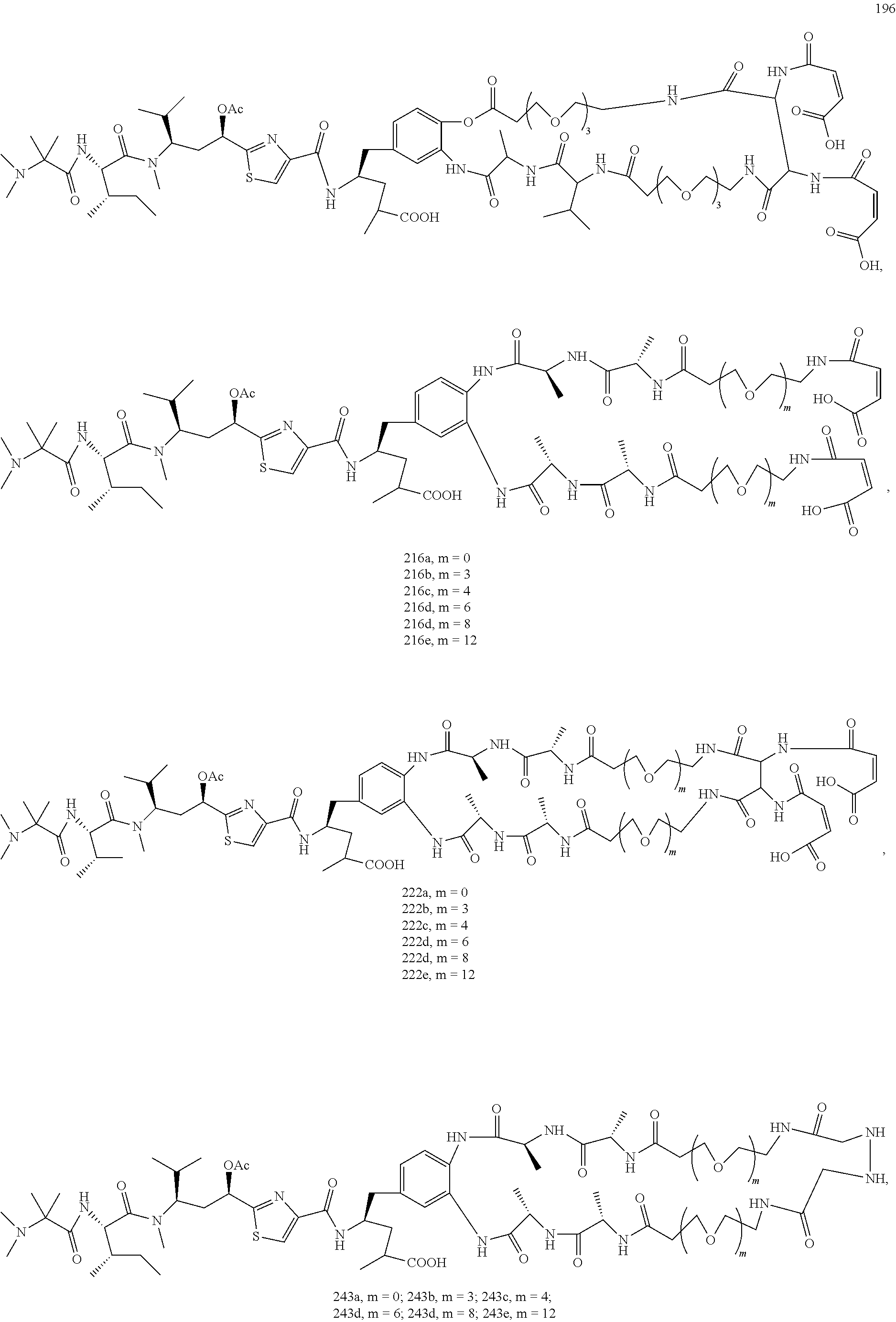

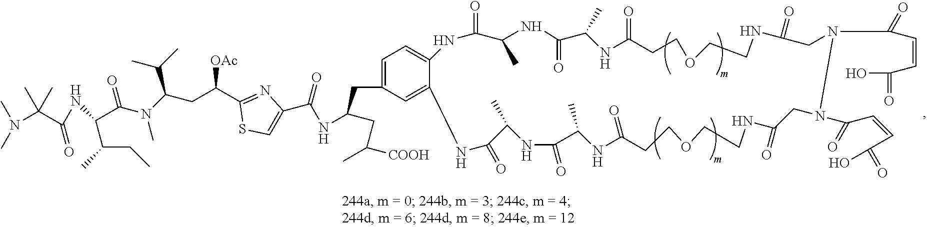

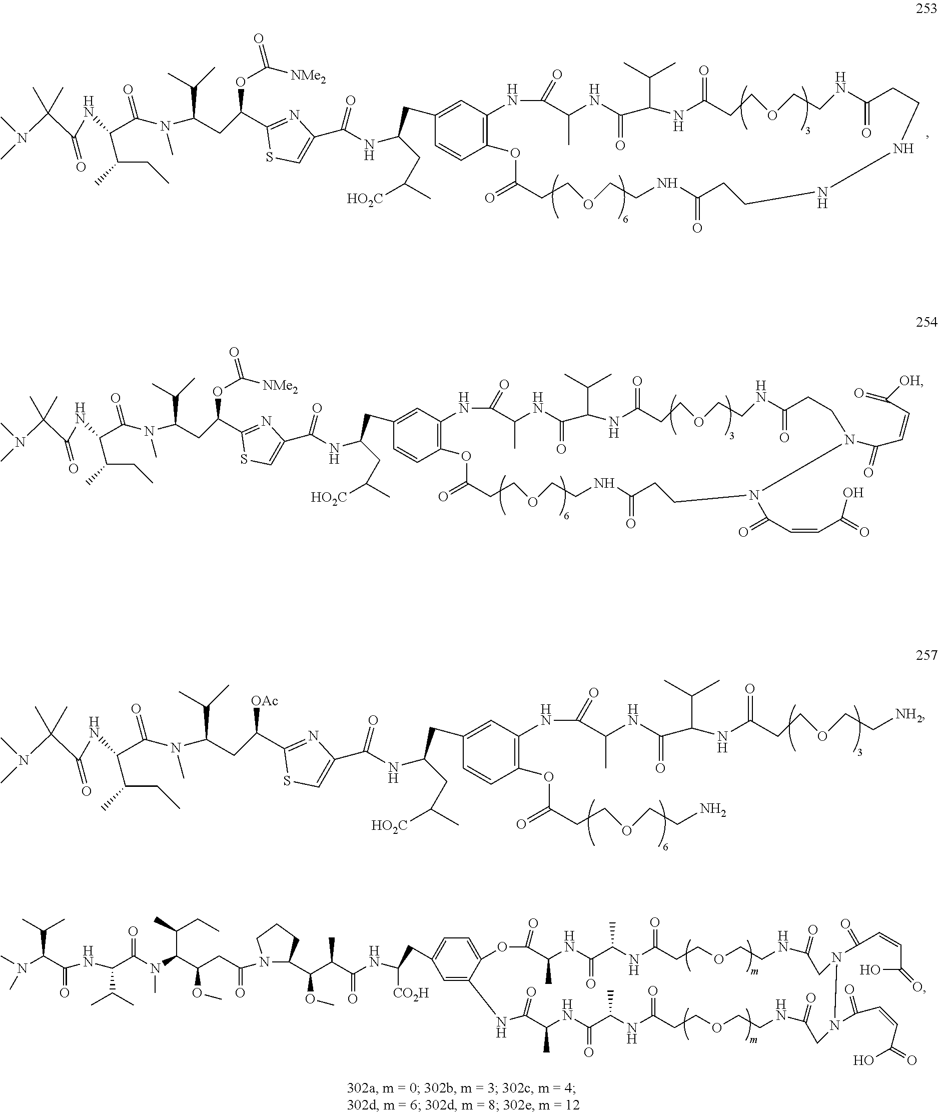

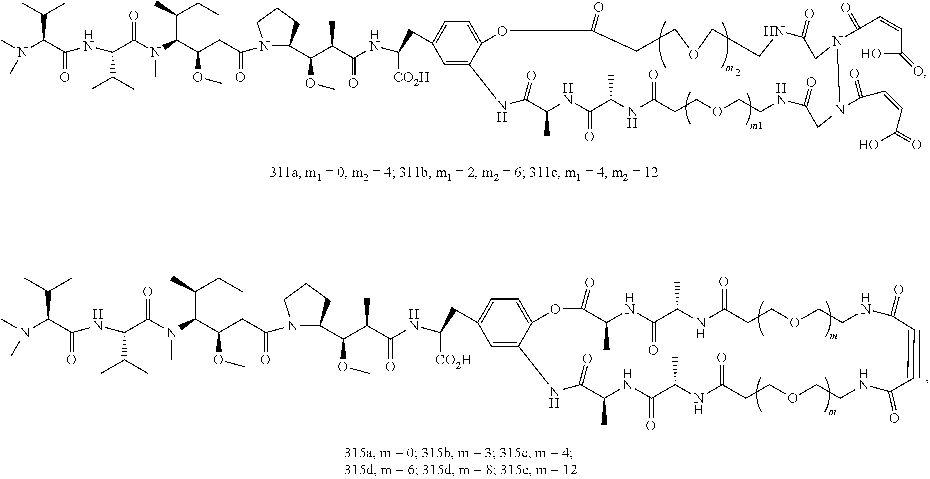

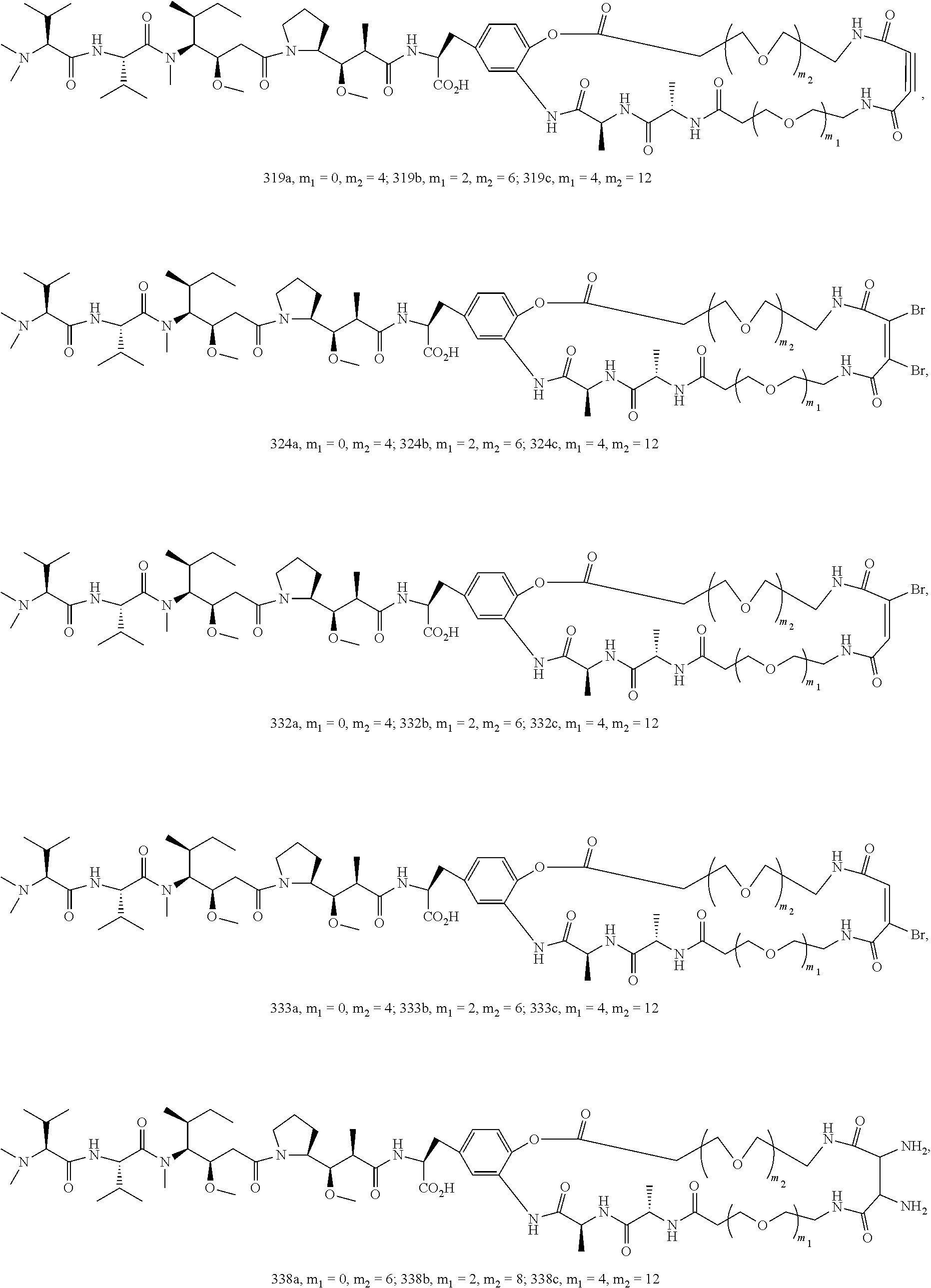

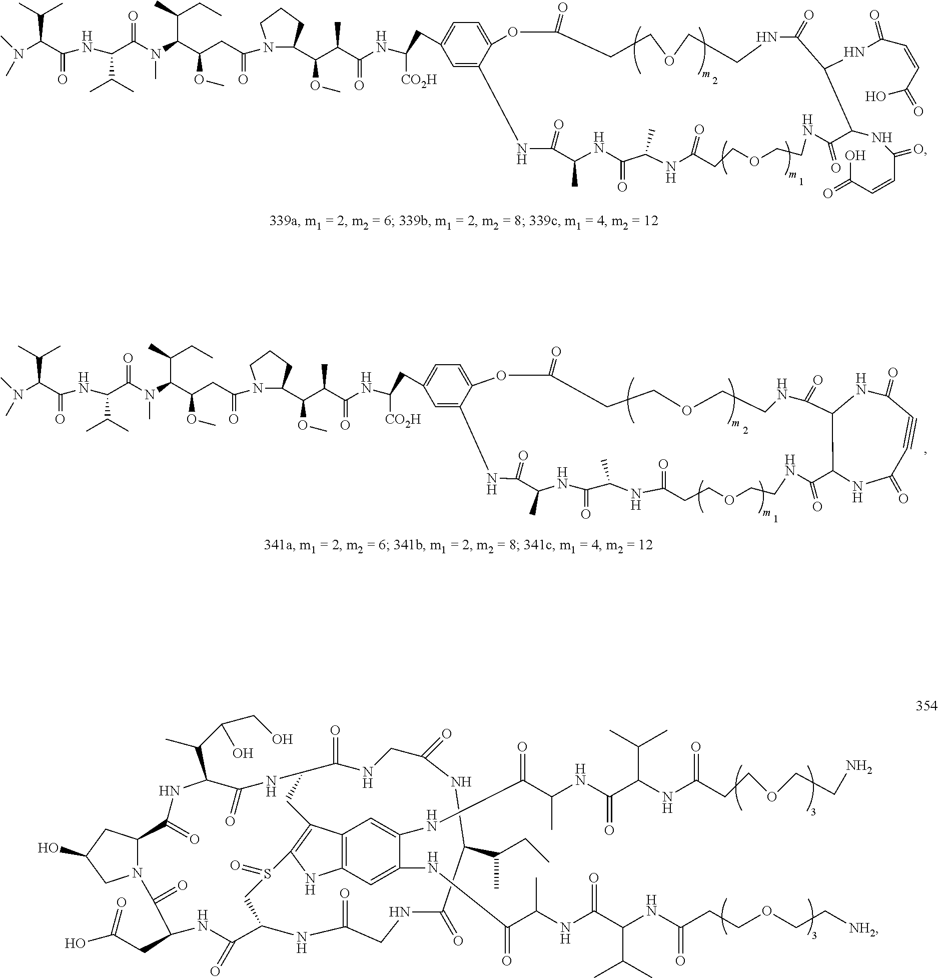

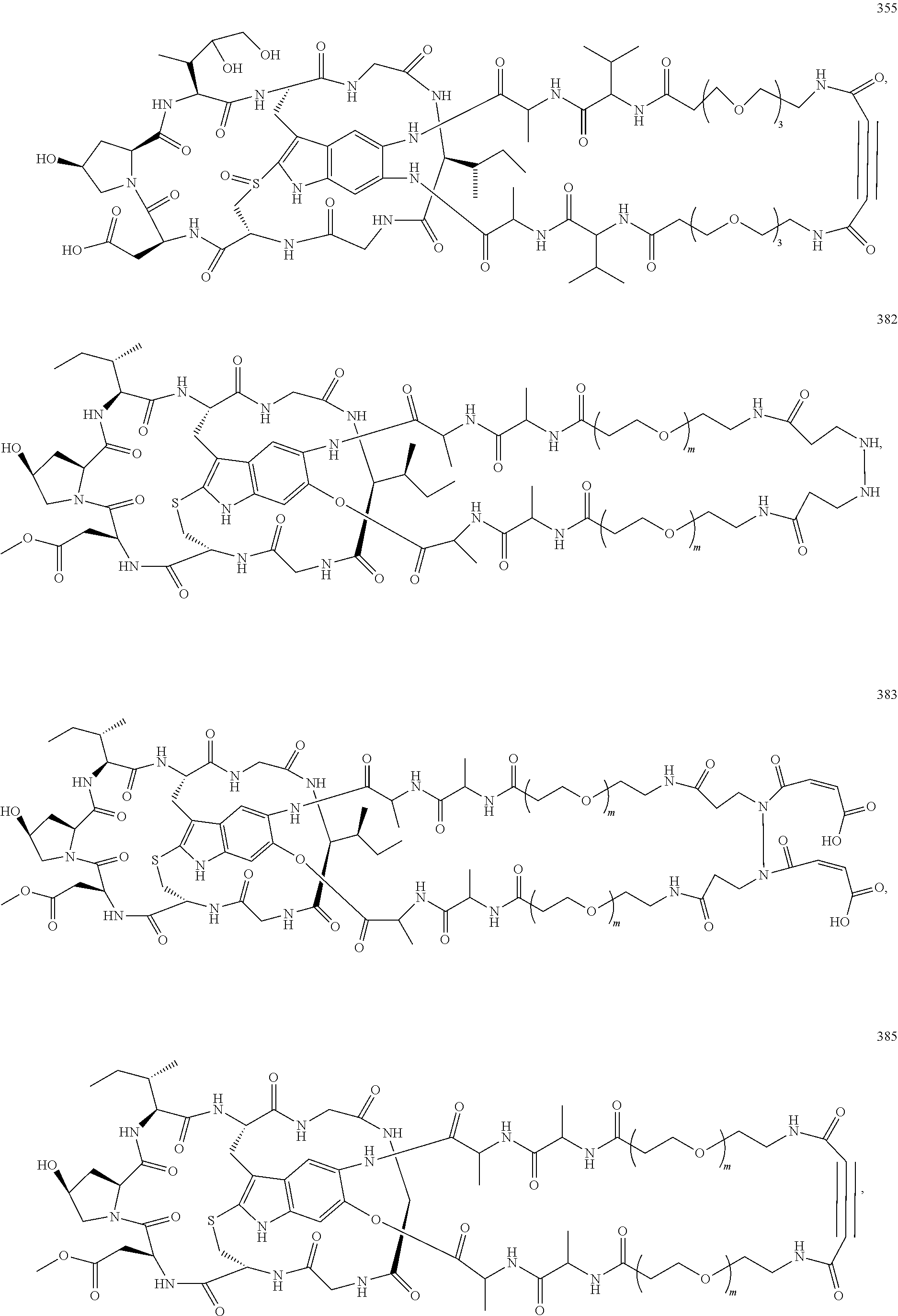

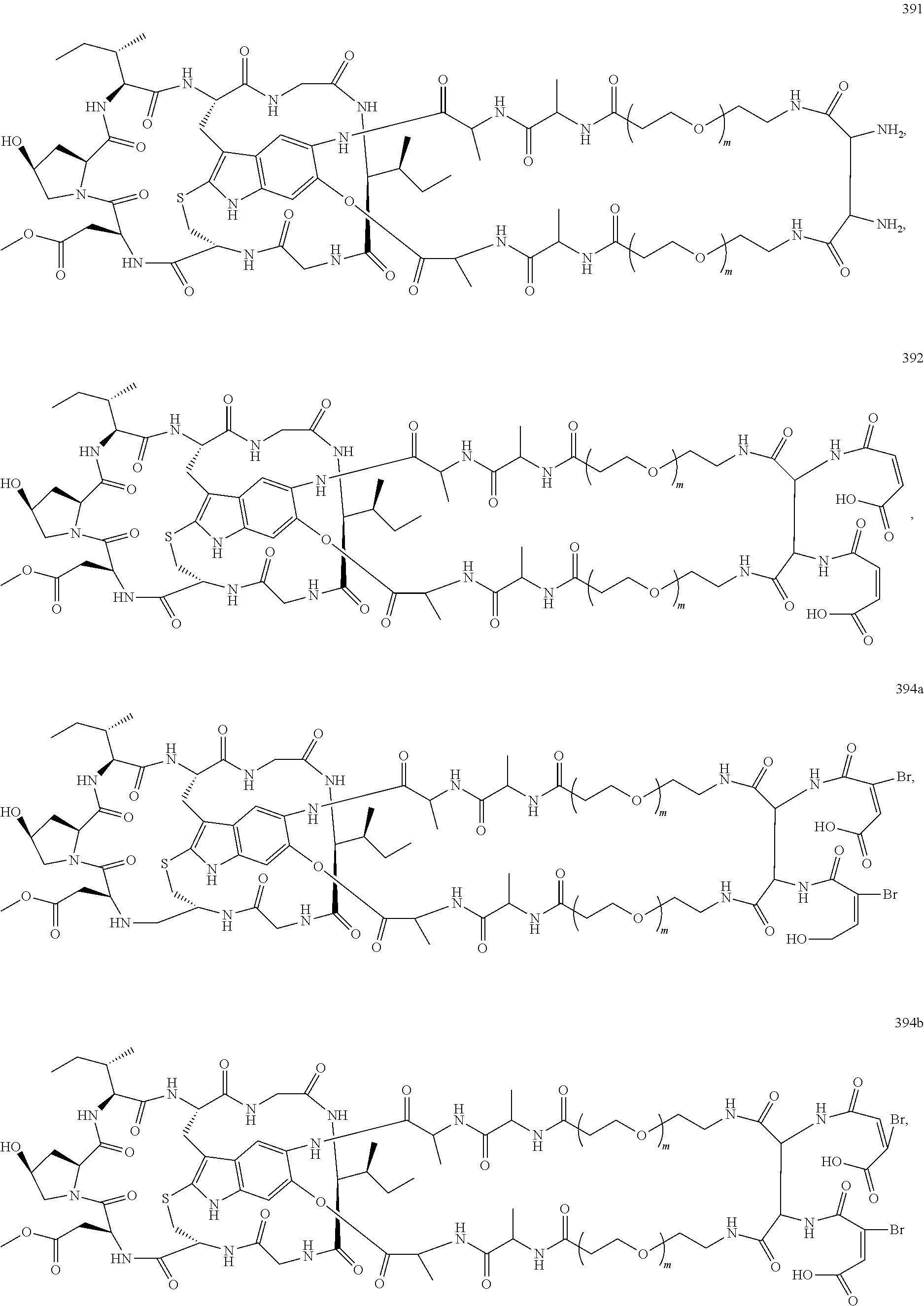

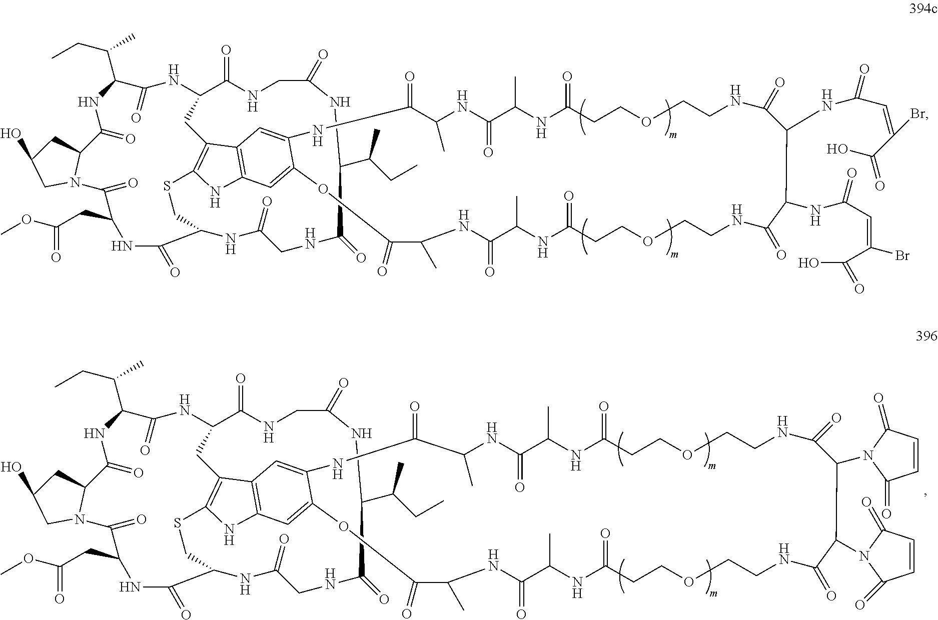

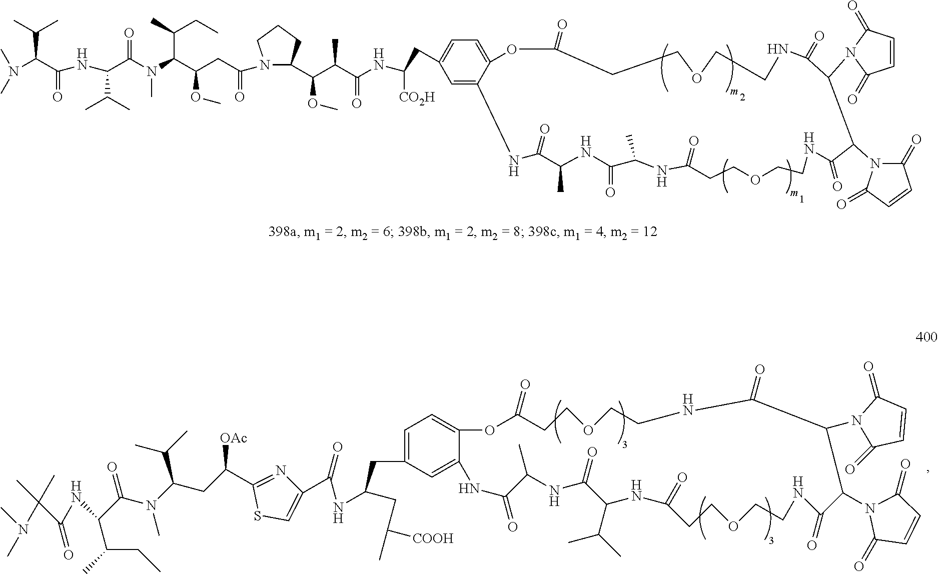

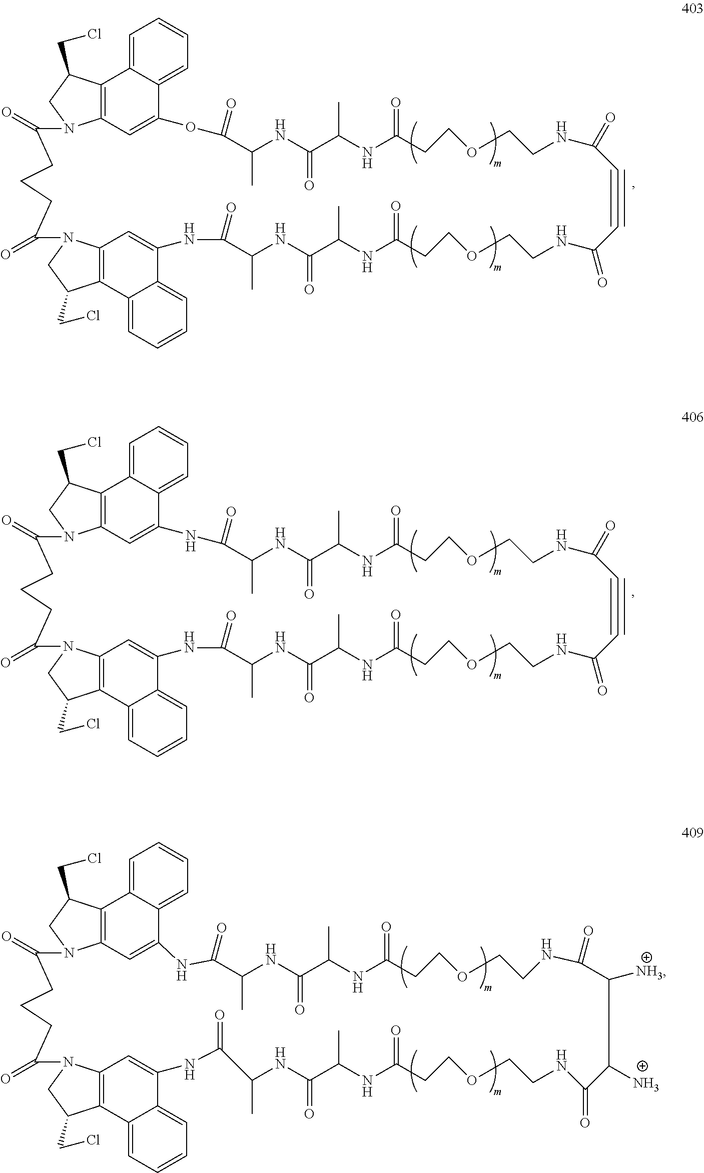

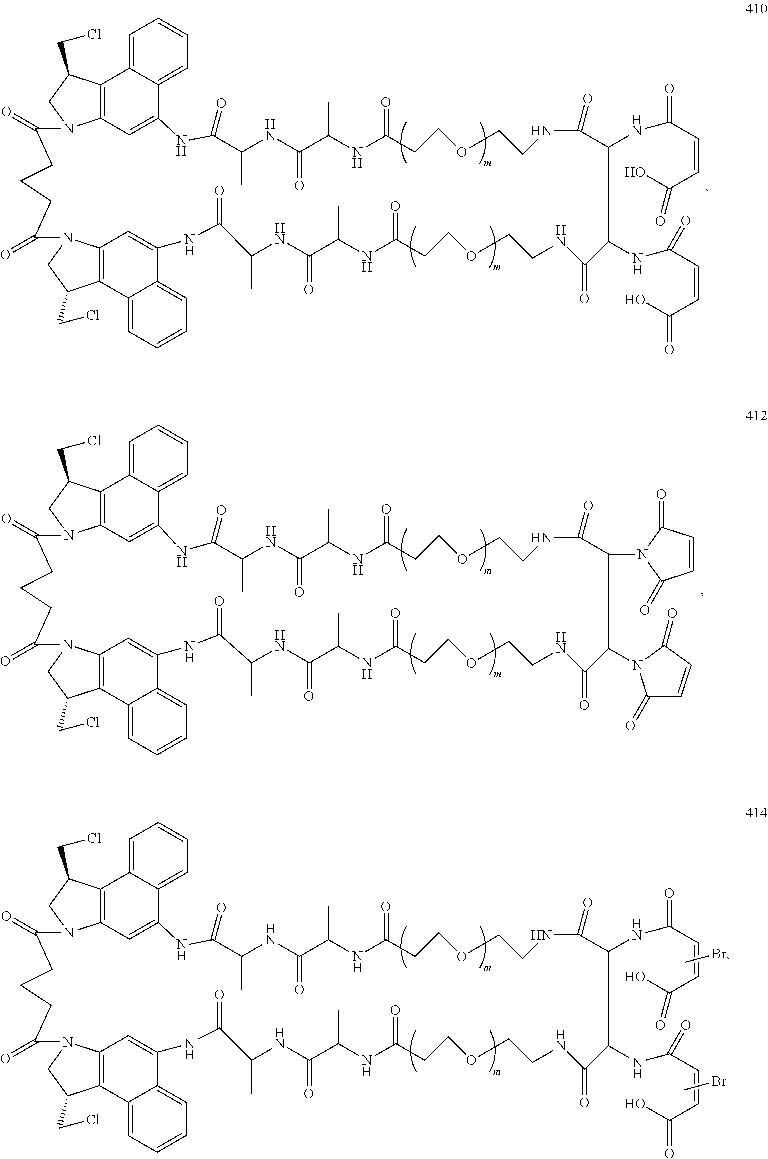

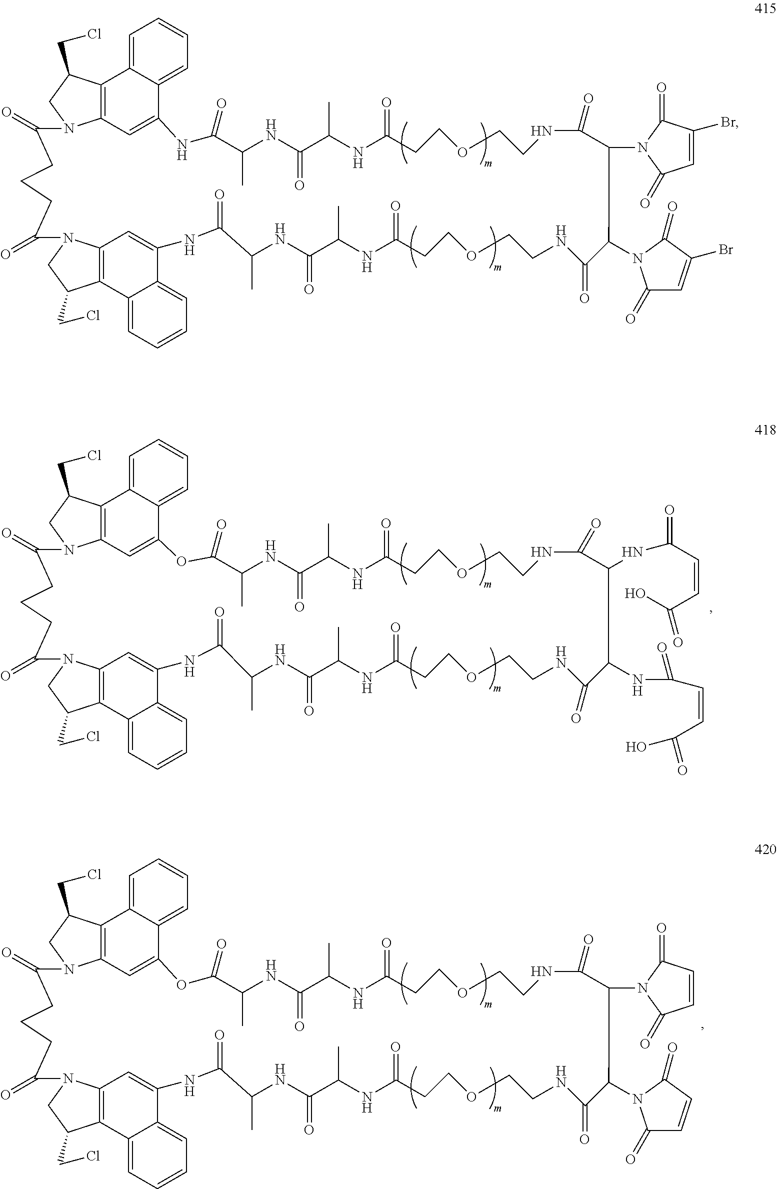

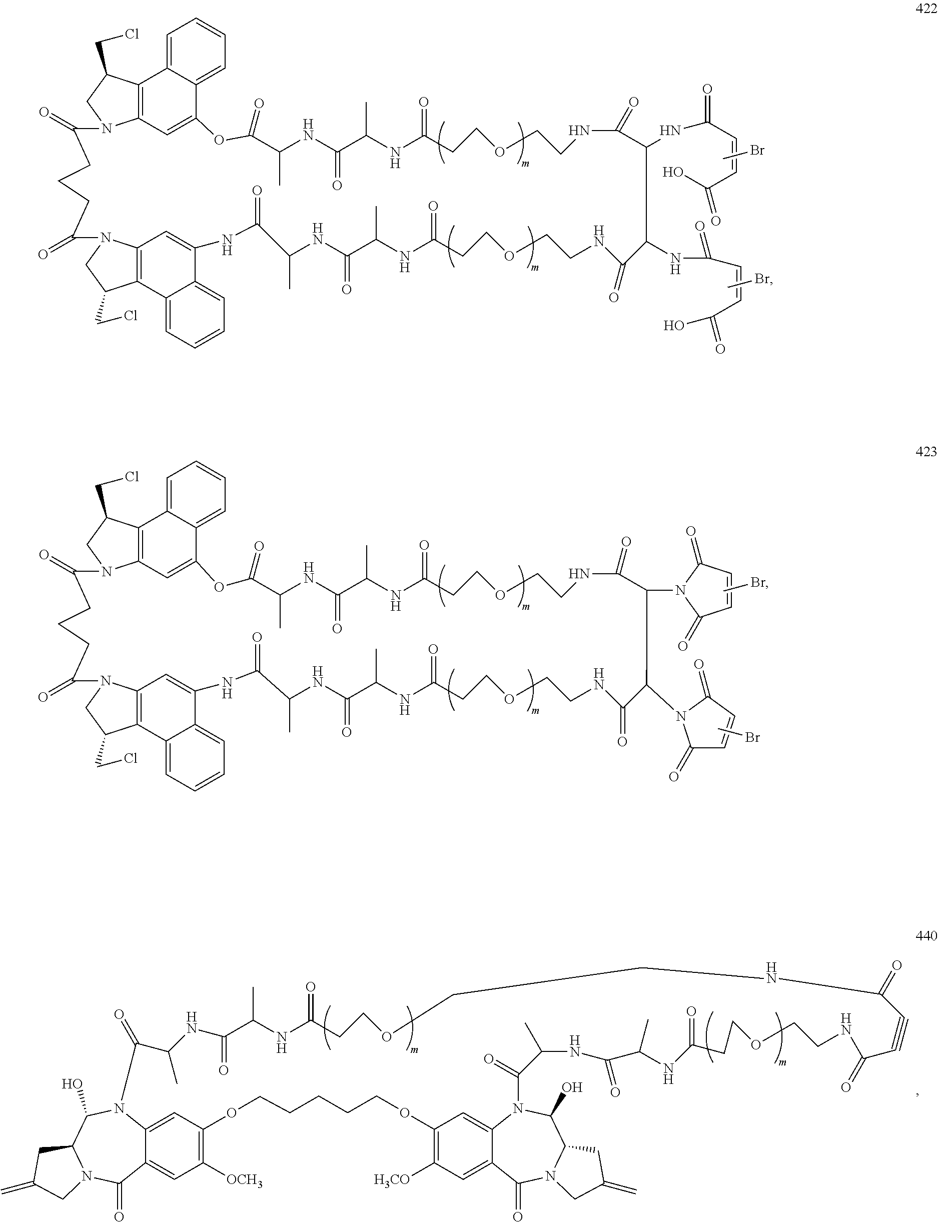

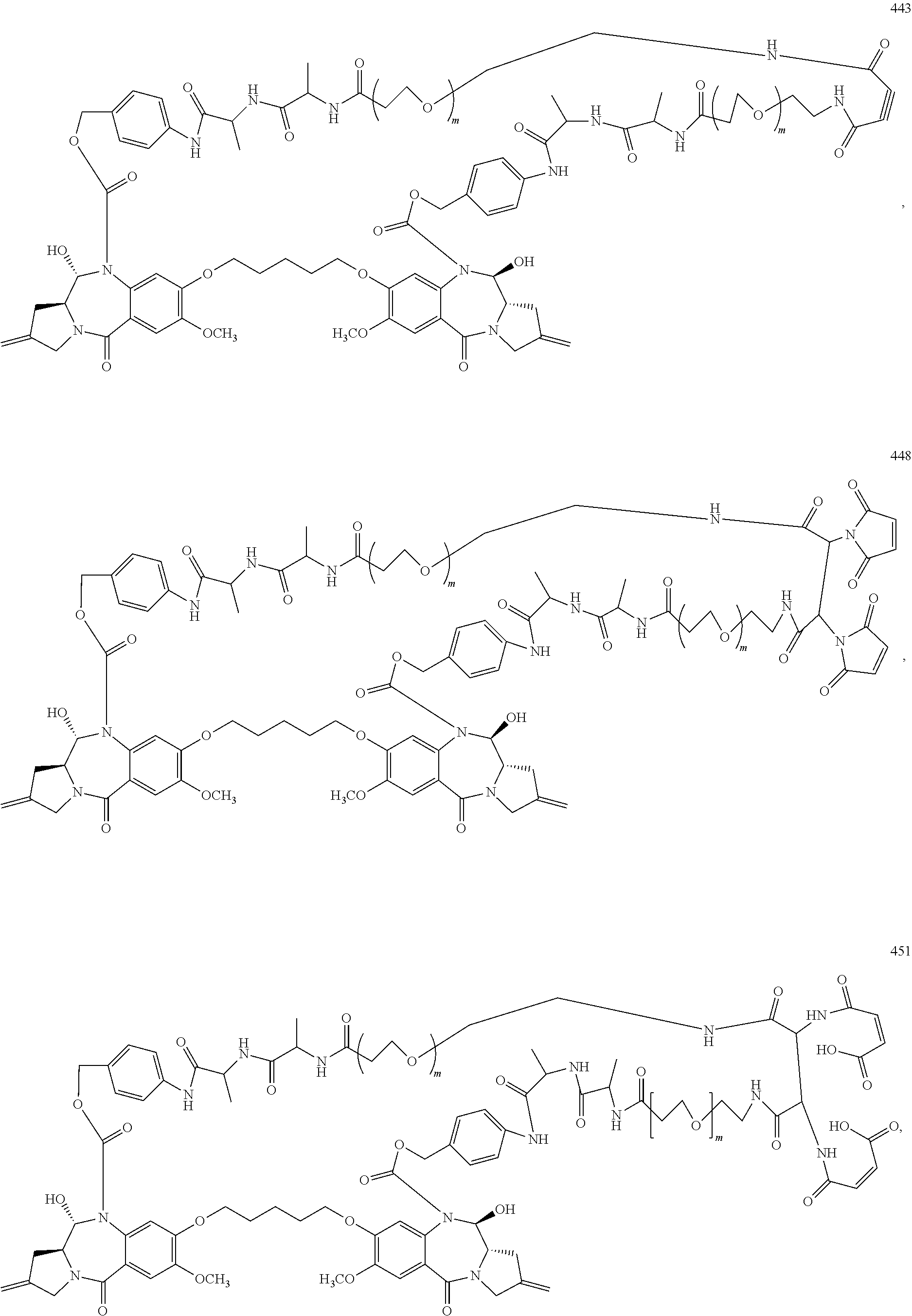

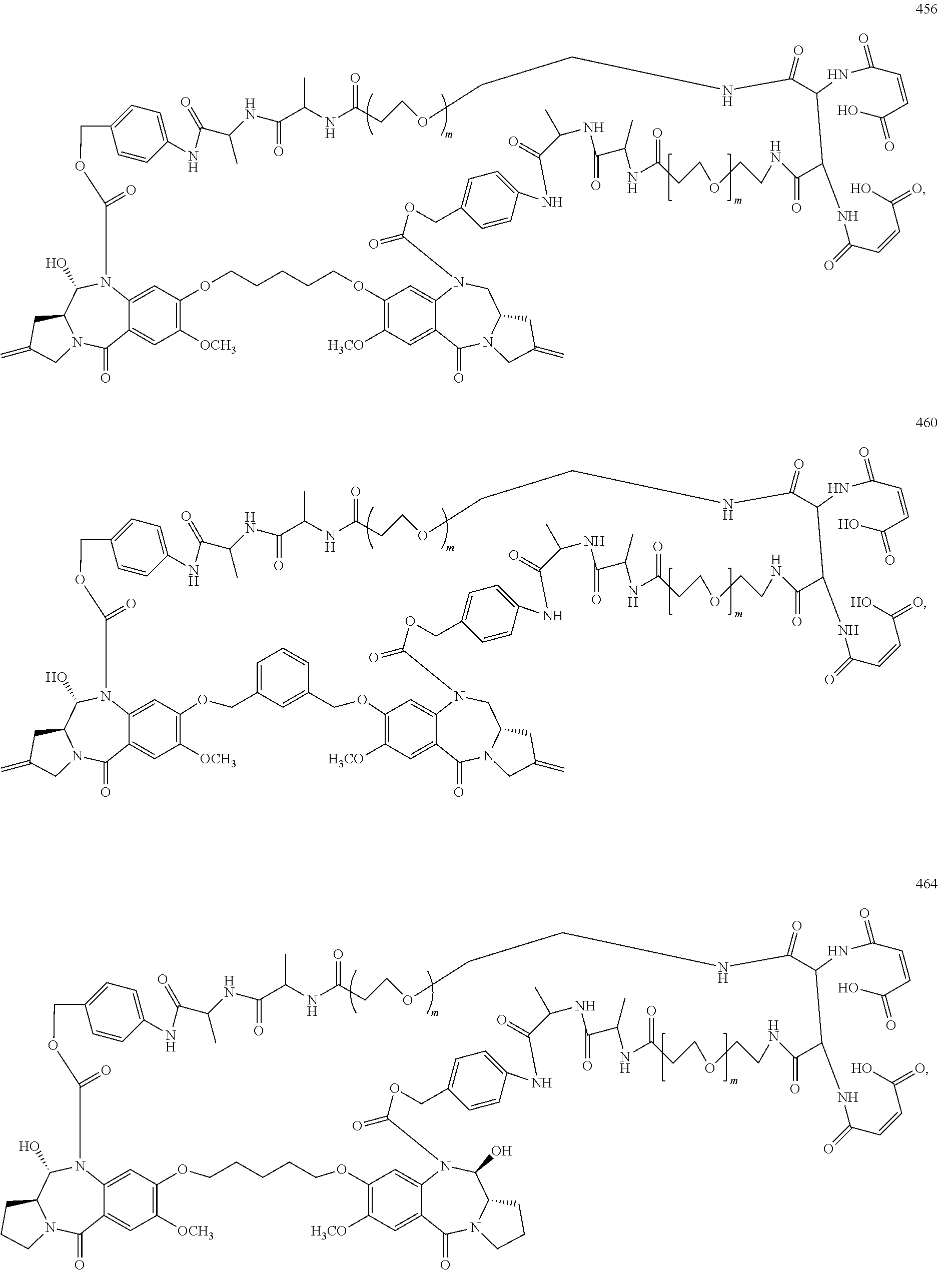

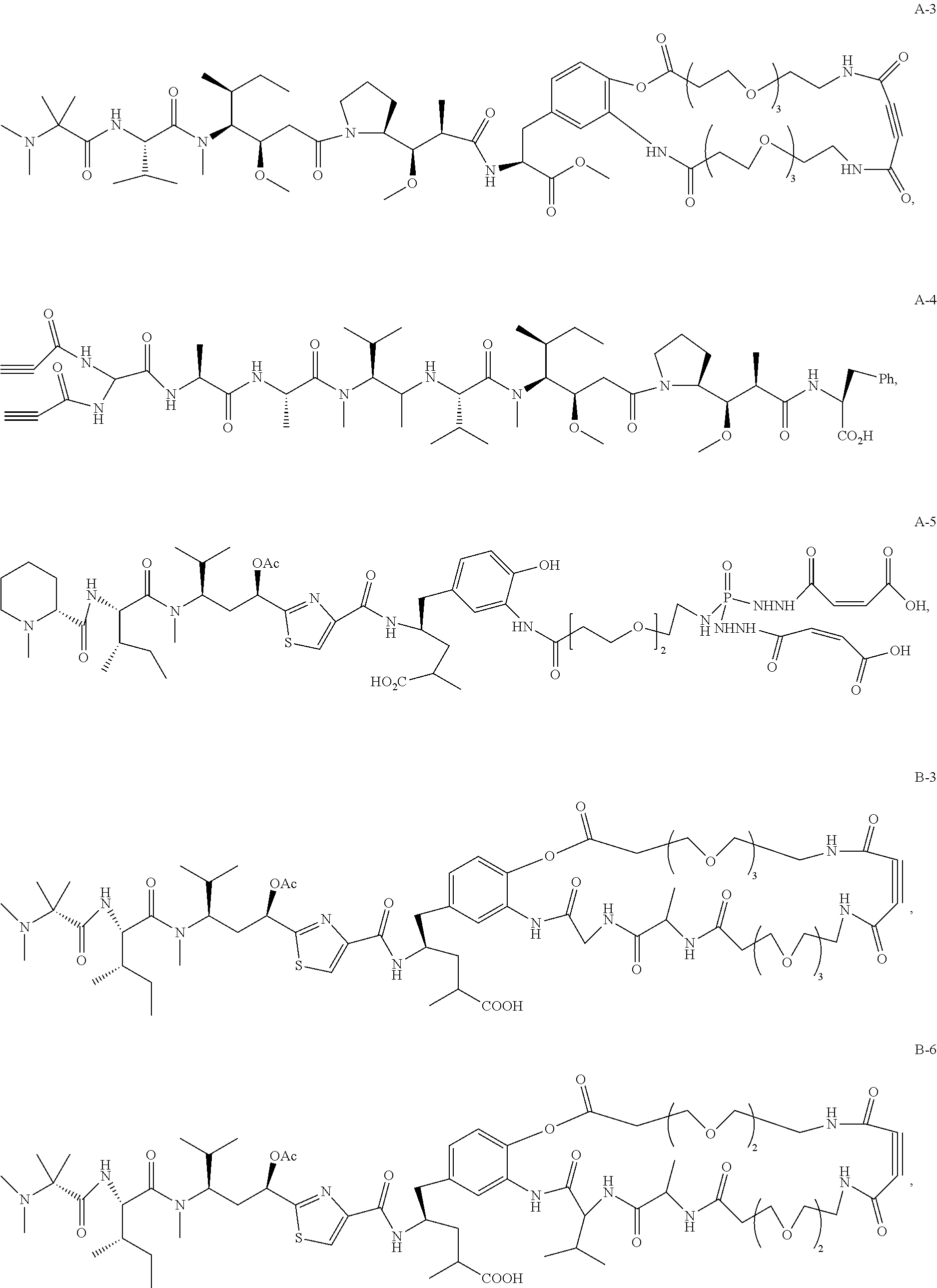

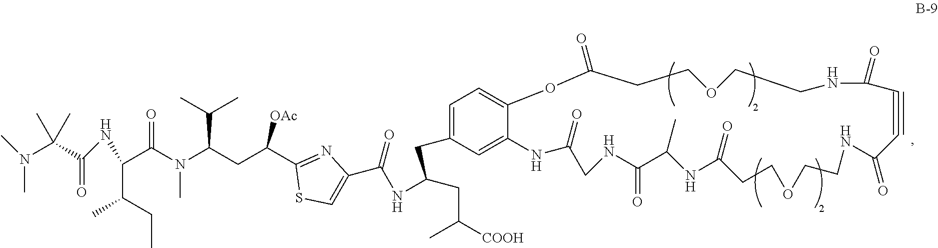

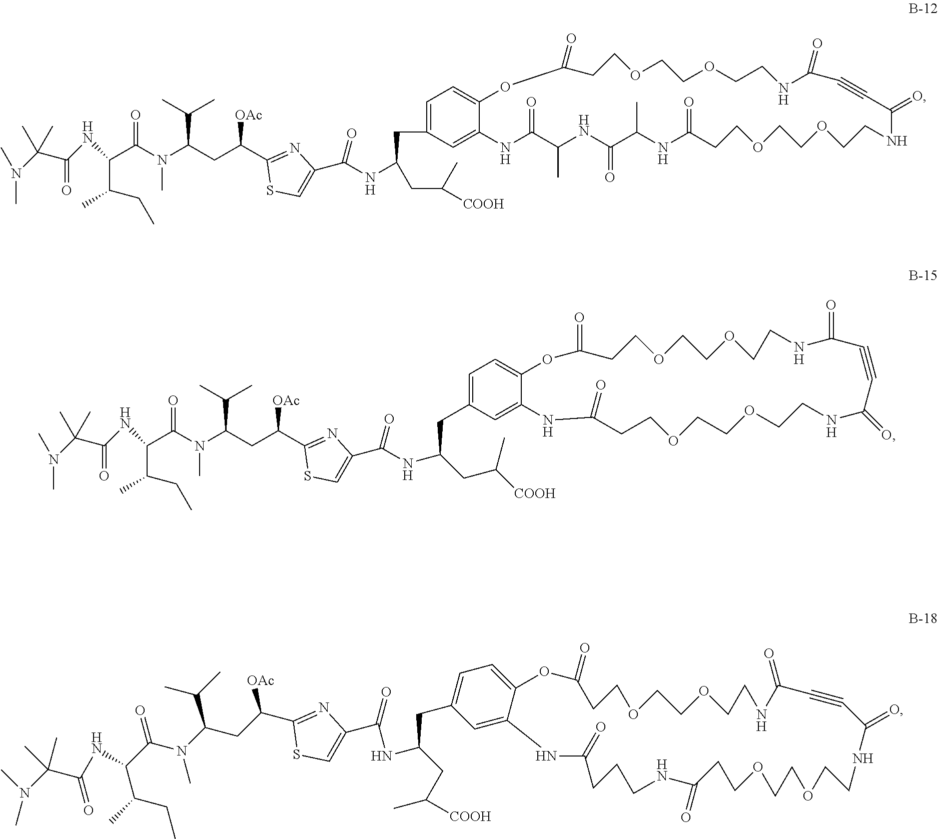

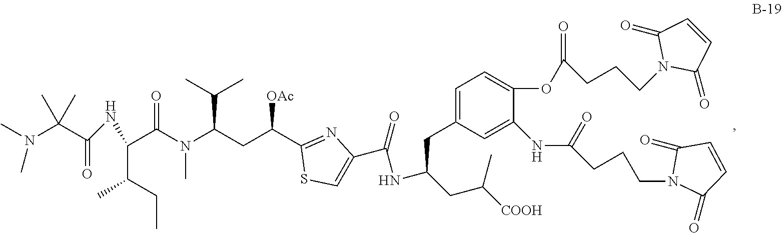 [SMILES: CC[C@H](C)[C@H](NC(=O)C(C)(C)N(C)C)C(=O)N(C)[C@H](C[C@@H](OC(C)=O)c1nc(C(=O)N[C@@H](Cc2ccc(OC(=O)CCCN3C(=O)C=CC3=O)c(NC(=O)CCCN3C(=O)C=CC3=O)c2)CC(C)C(=O)O)cs1)C(C)C]

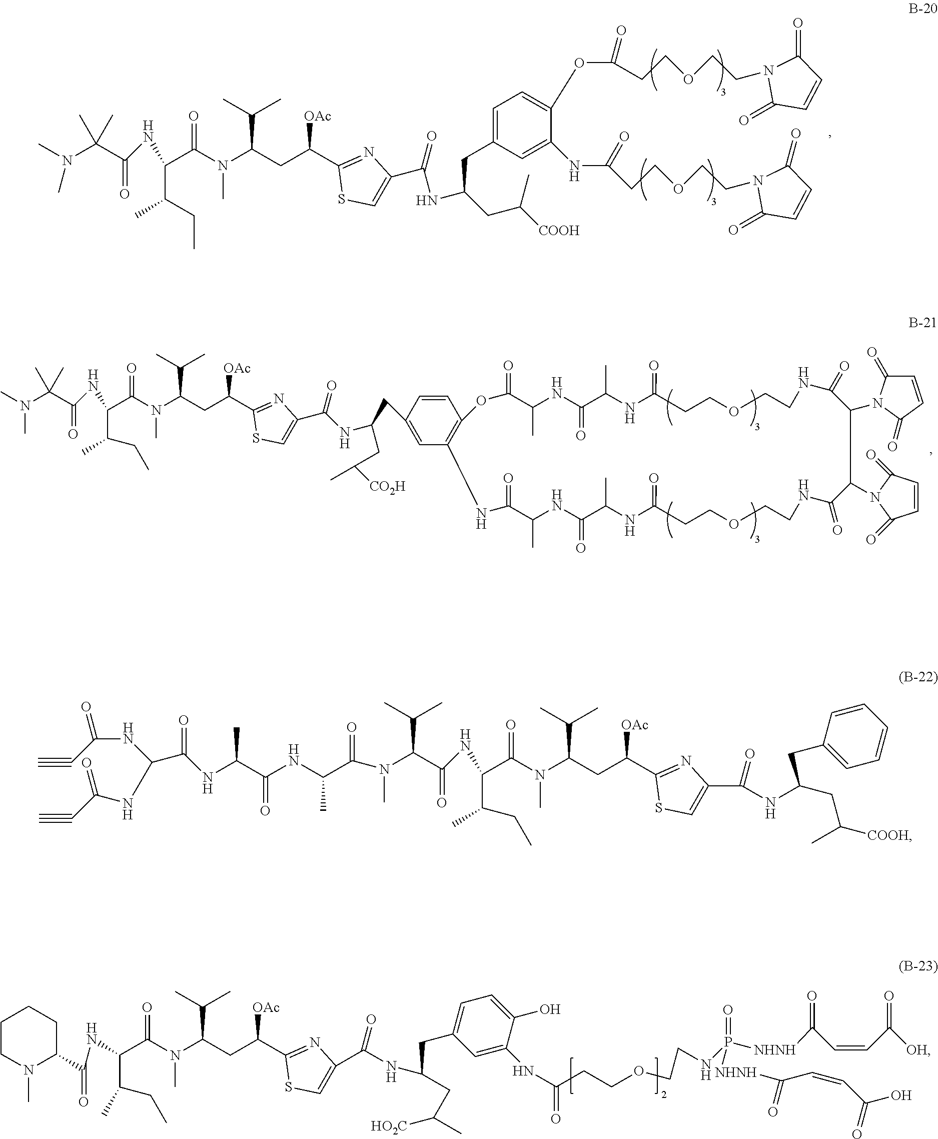

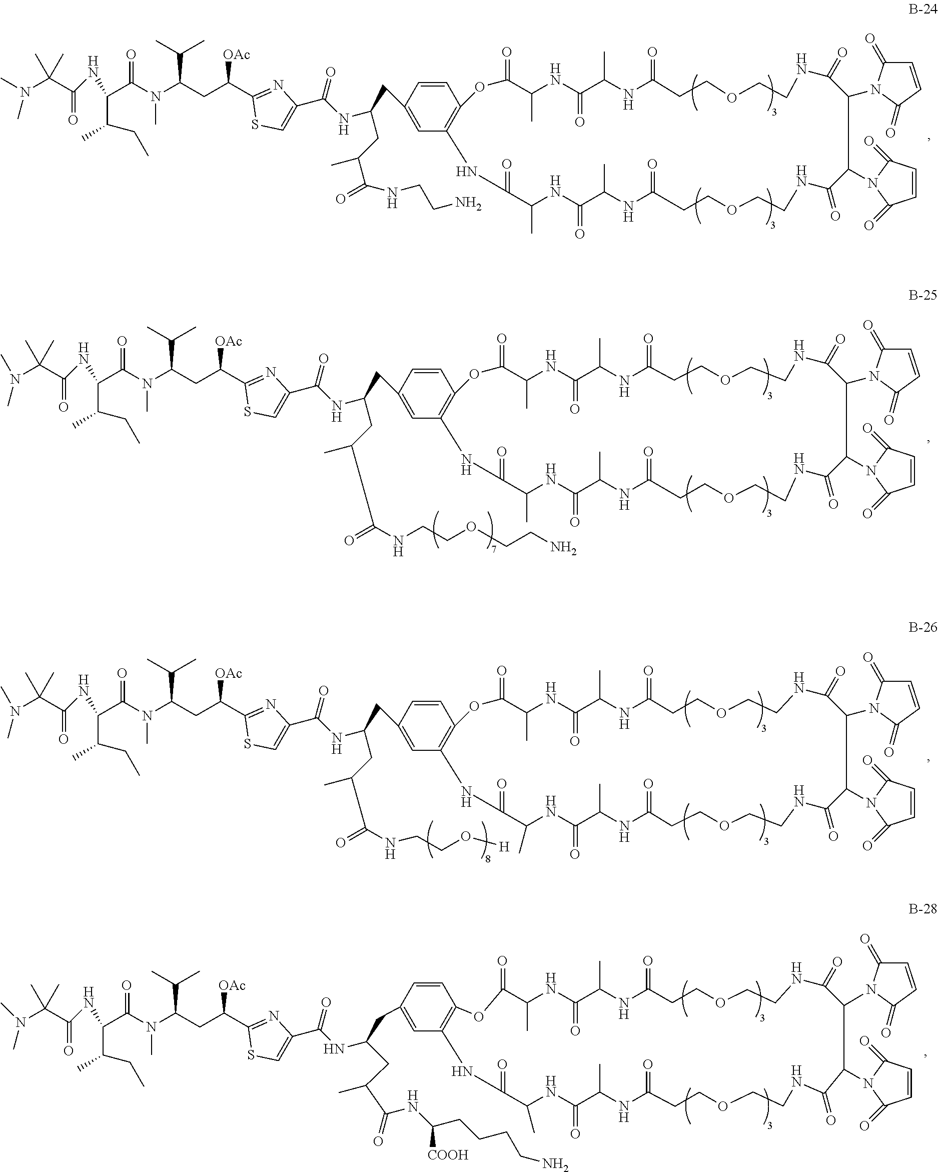

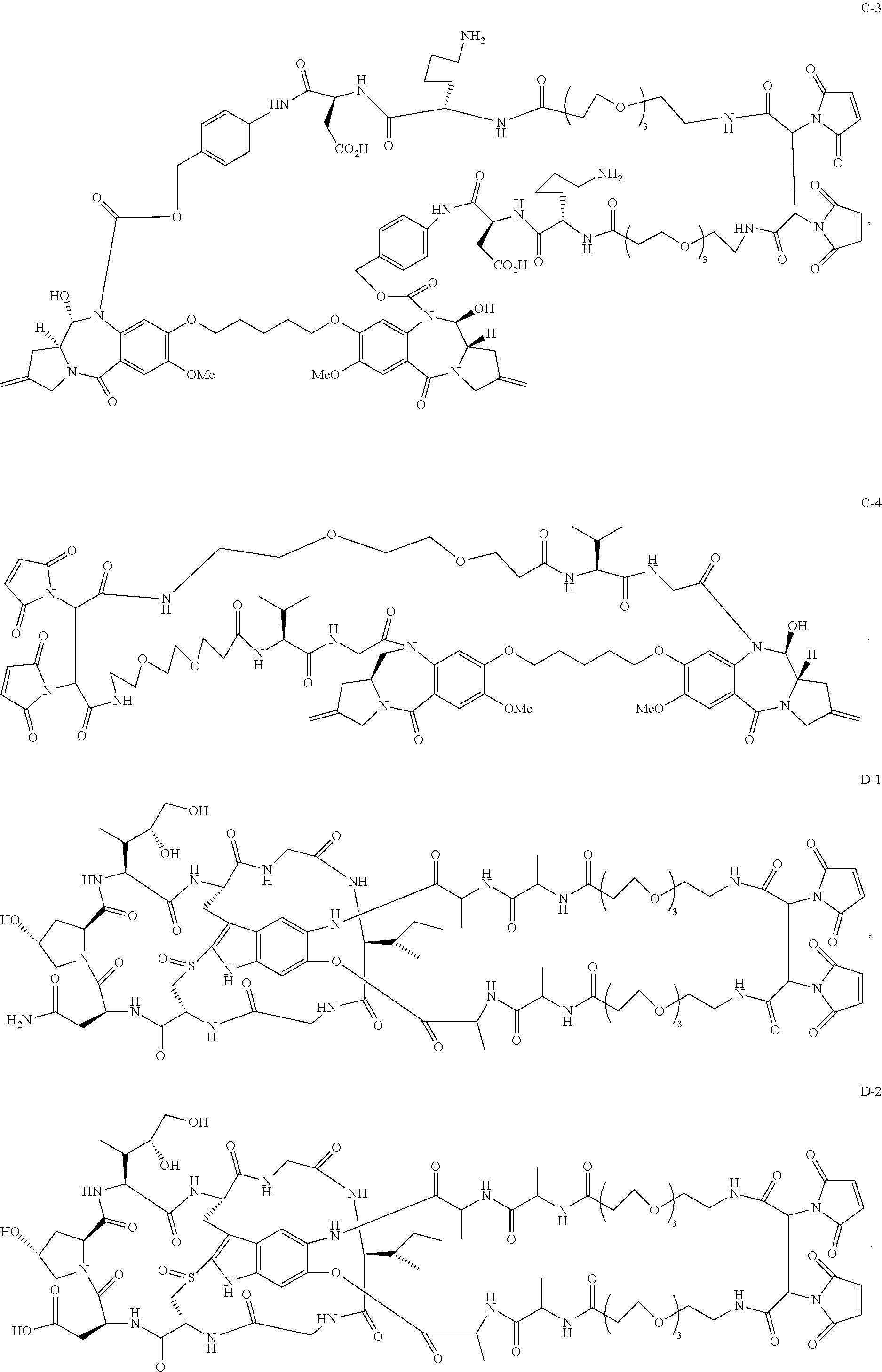

36. The conjugate compound of Formula (I) according to claim 1, having a Formula of 103, 105, 113, 117, 120, 127, 129, 131, 133, 135, 140, 142, 150, 152, 169, 177, 186, 190, 197, 217a, 217b, 217c, 217d, 217e, 217f, 223a, 223b, 223c, 223d, 223e, 223f, 245a, 245b, 245c, 245d, 245e, 245f, 255, 303a, 303b, 303c, 303d, 303e, 303f, 312a, 312b, 312c, 316a, 316b, 316c, 316d, 316e, 316f, 320a, 320b, 320c, 325a, 325b, 325c, 340a, 340b, 340c, 342a, 342b, 342c, 356, 384, 386, 393, 395a, 395b, 397, 399a, 399b, 399c, 401, 404, 407, 411, 413, 416, 419, 421, 424, 441, 449, 452, 457, 461, 465, A-3a, A-4a, A-5a, B-3a, B-6a, B-9a, B-12a, B-15a, B-18a, B-19a, B-20a, B-21a, B-22a, B-23a, B-24a, B-25a, B-26a, B-28a, C-3a, C-4a, D-1a or D-2a as shown in following structures: 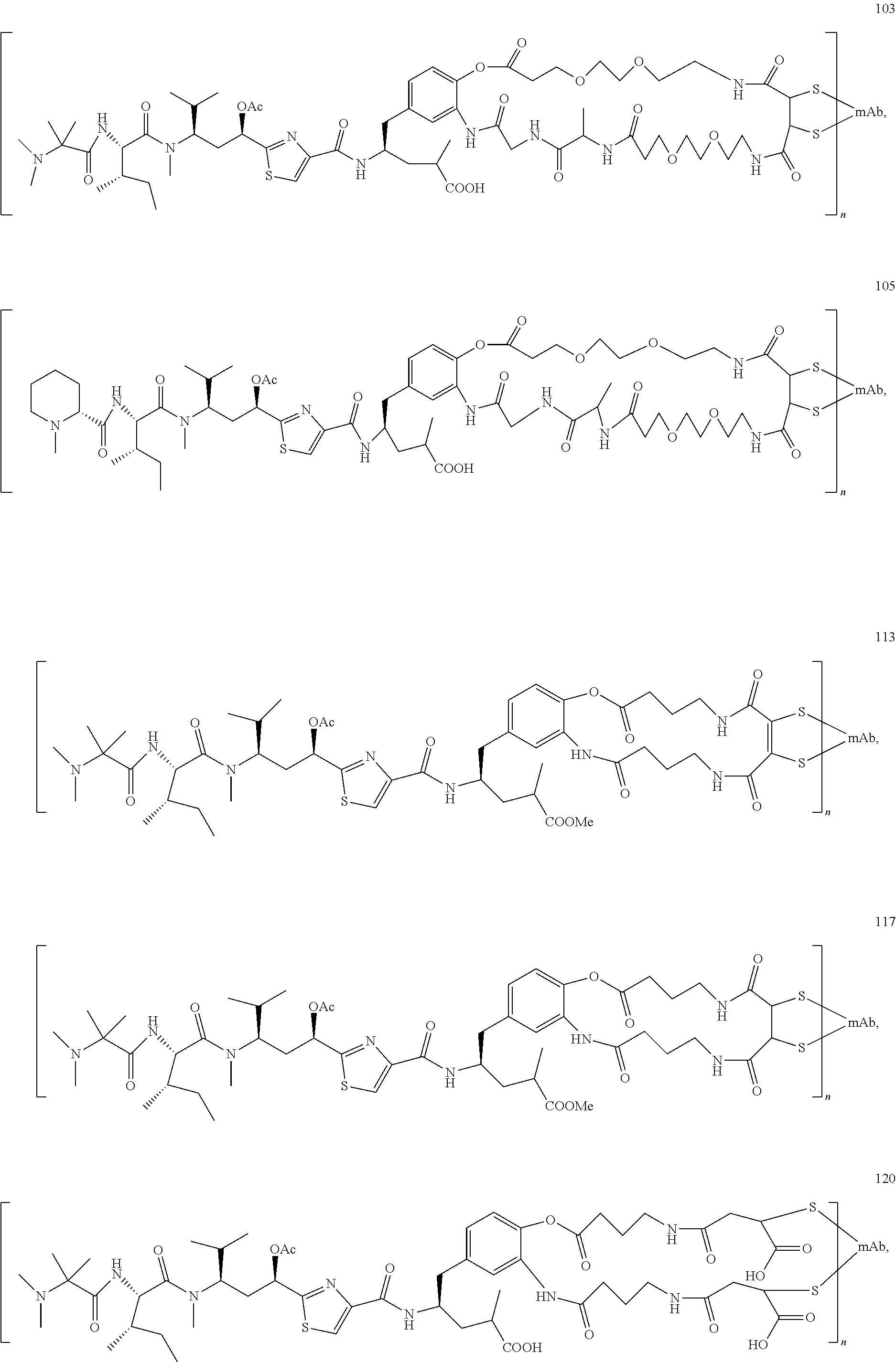 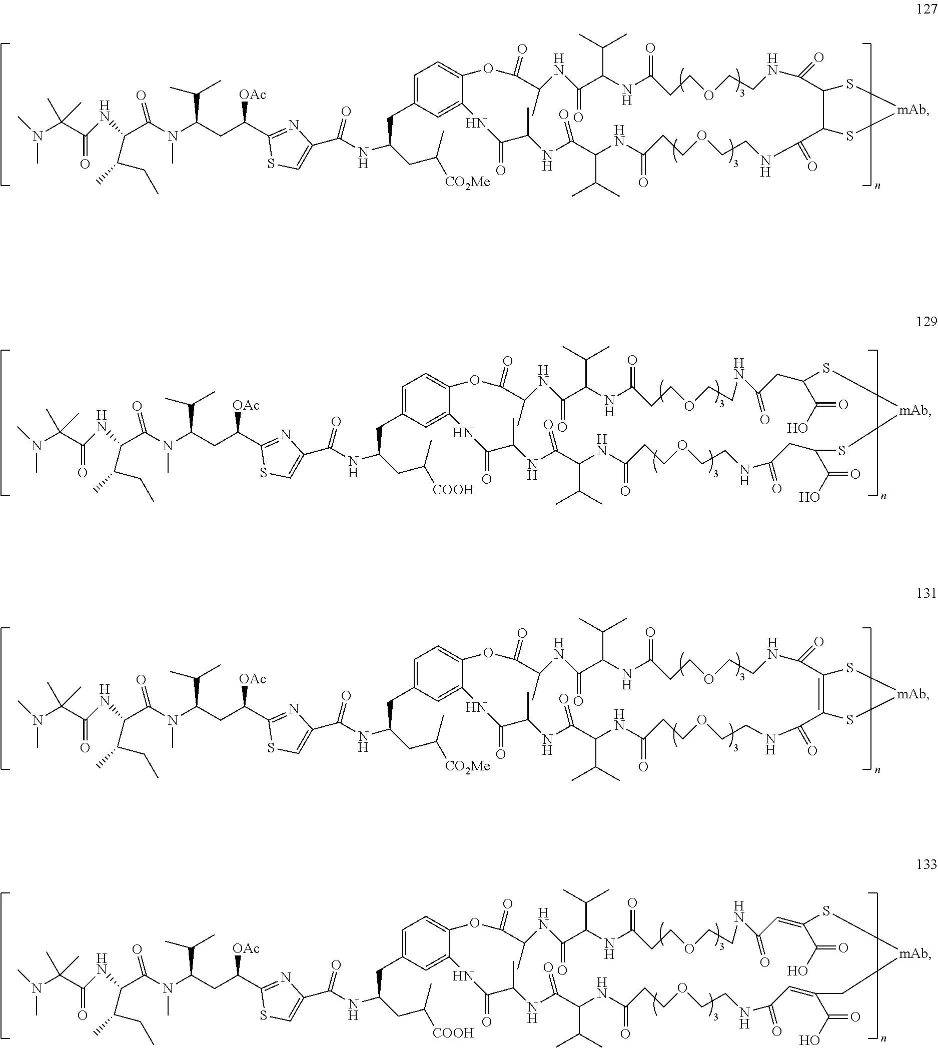 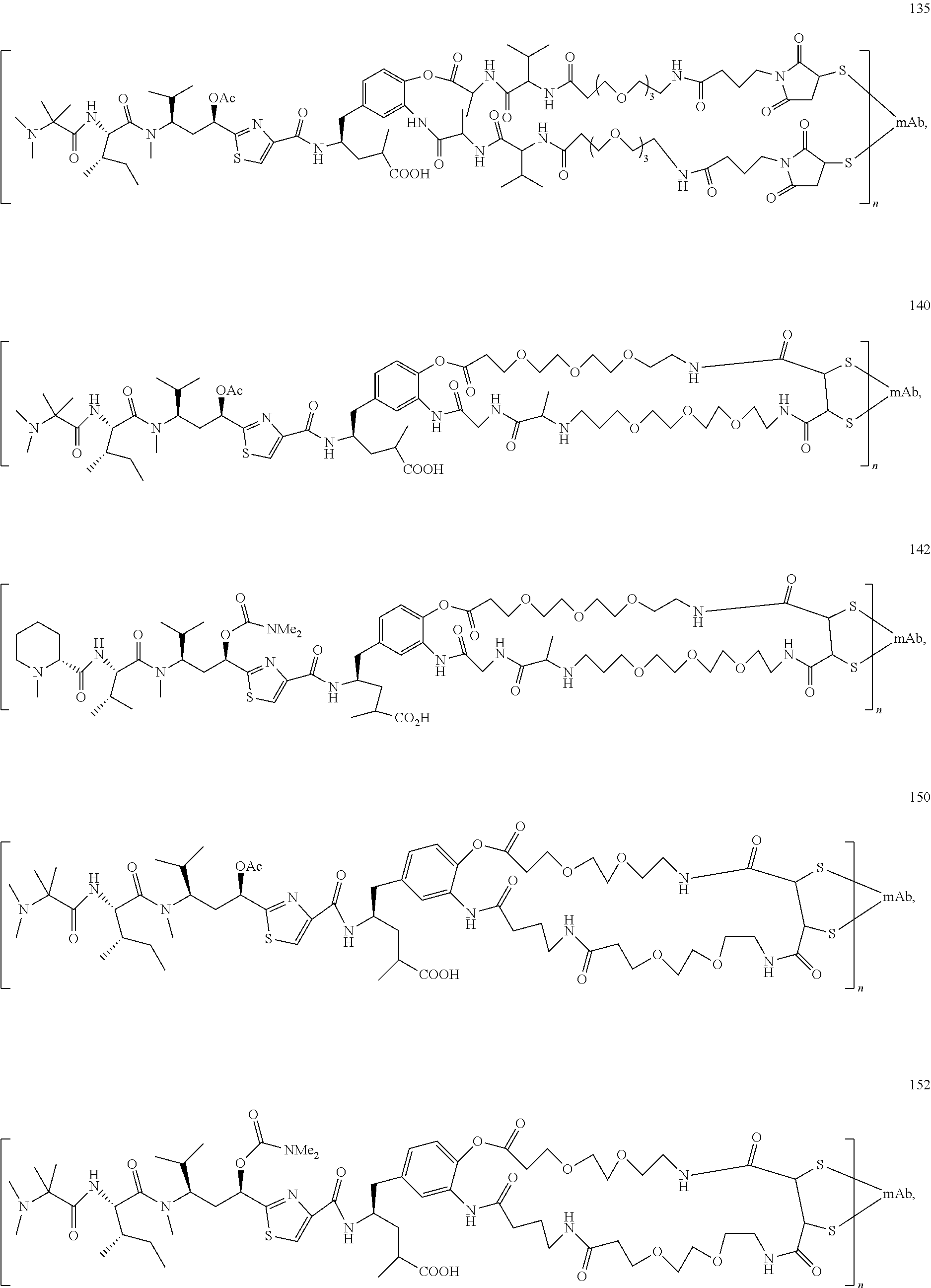 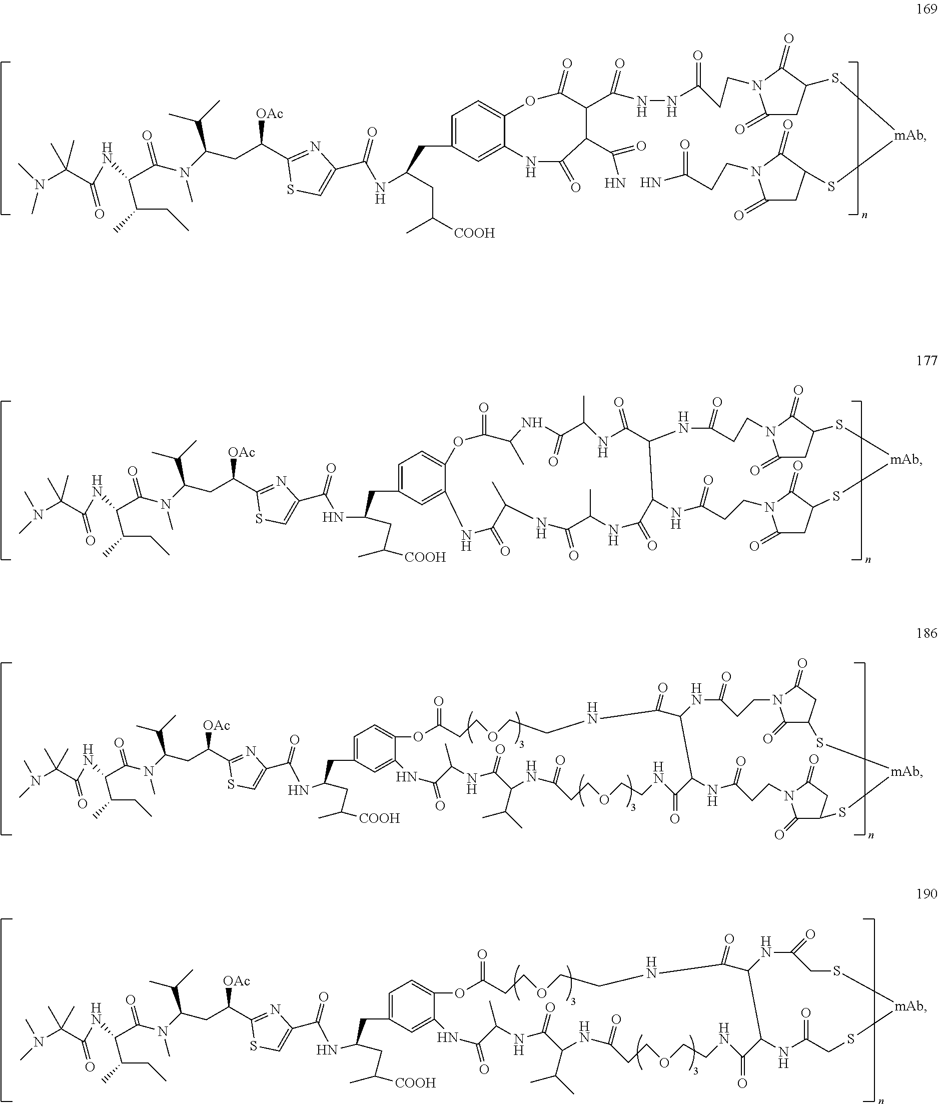 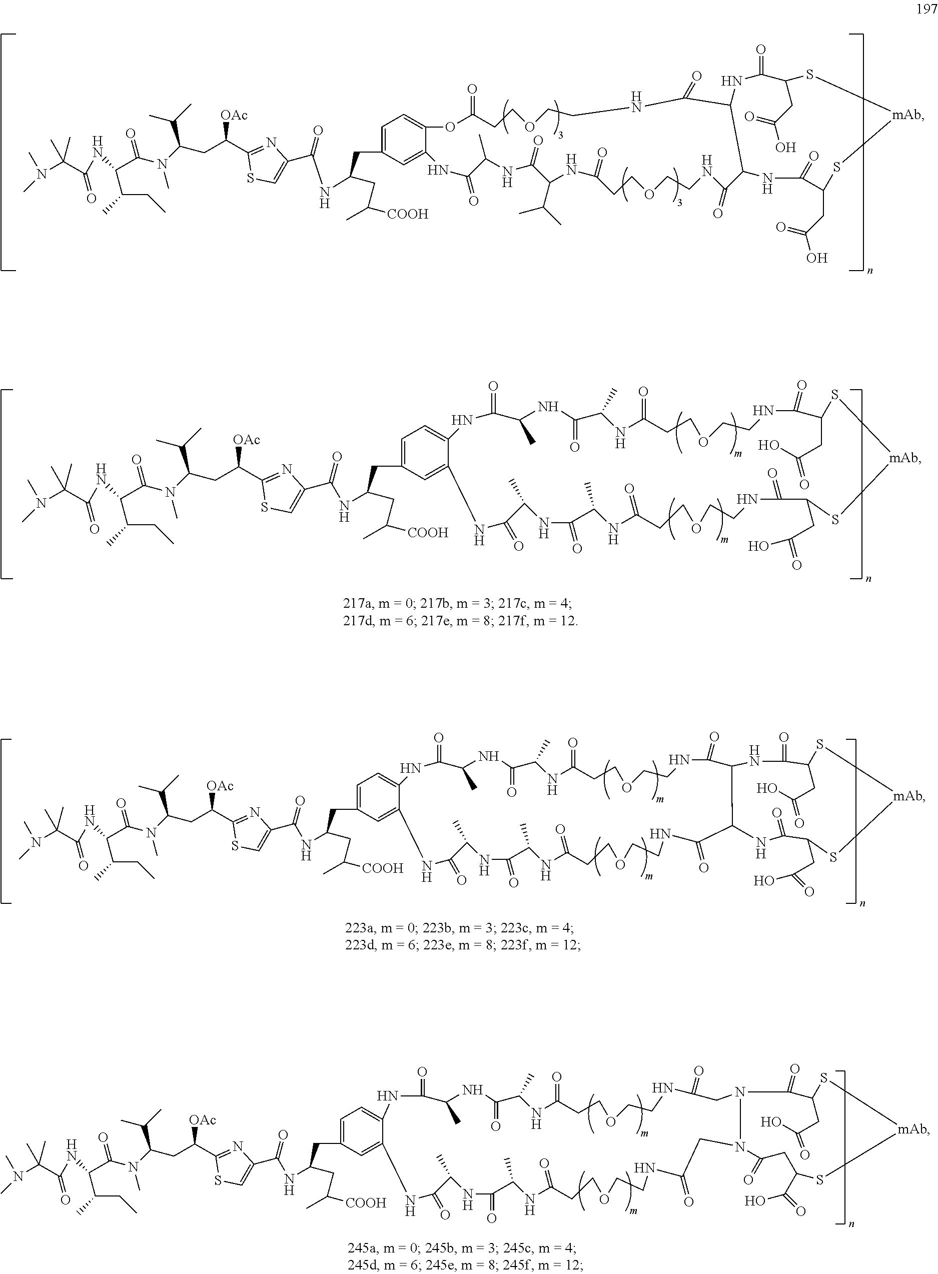 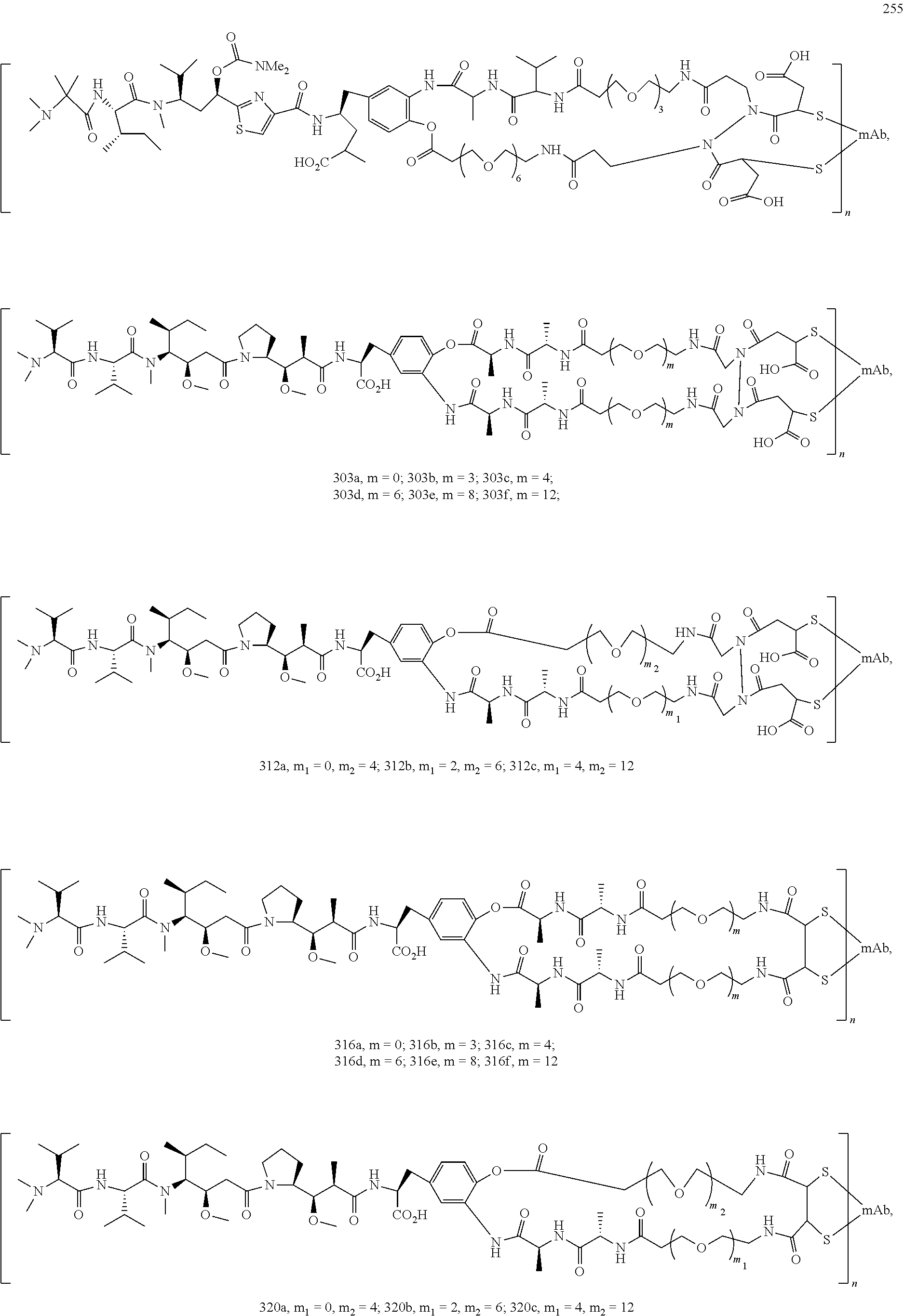 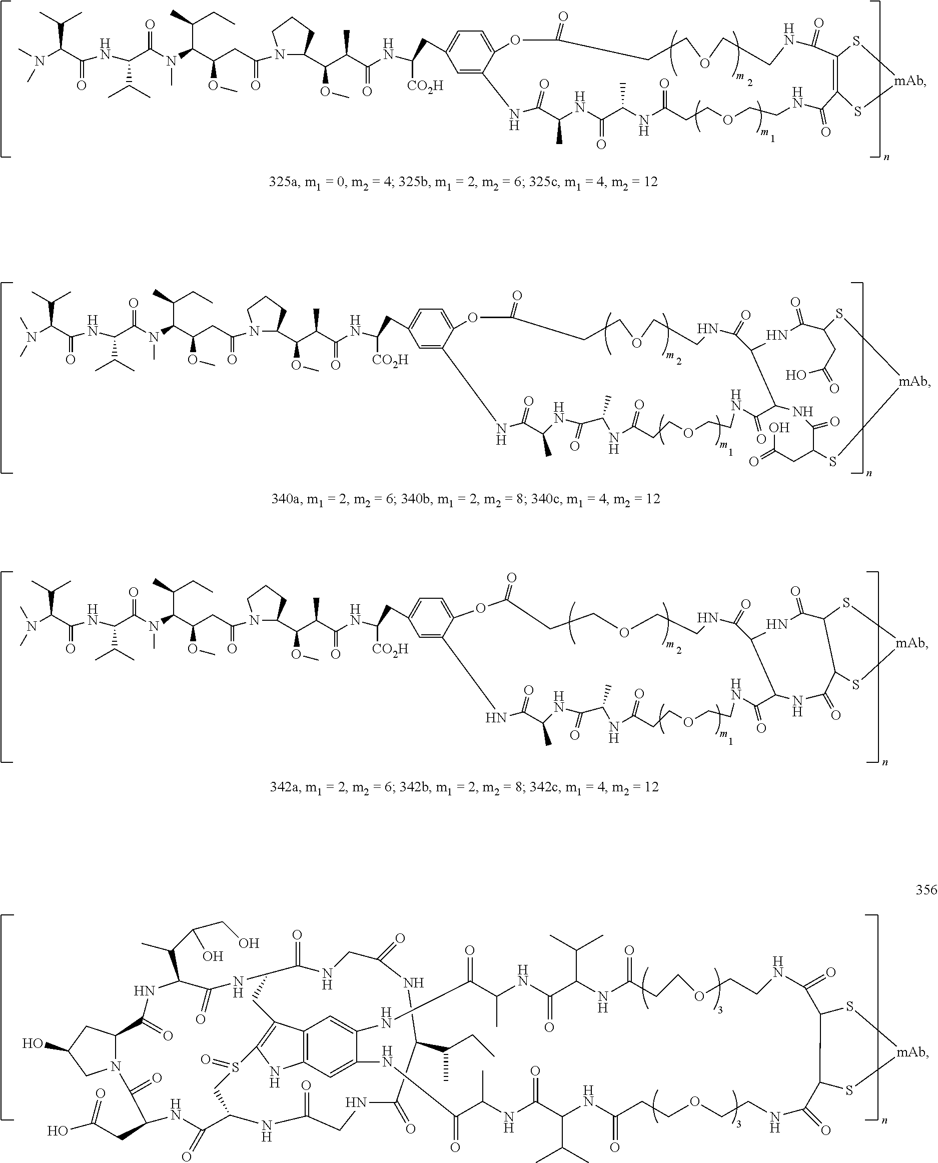 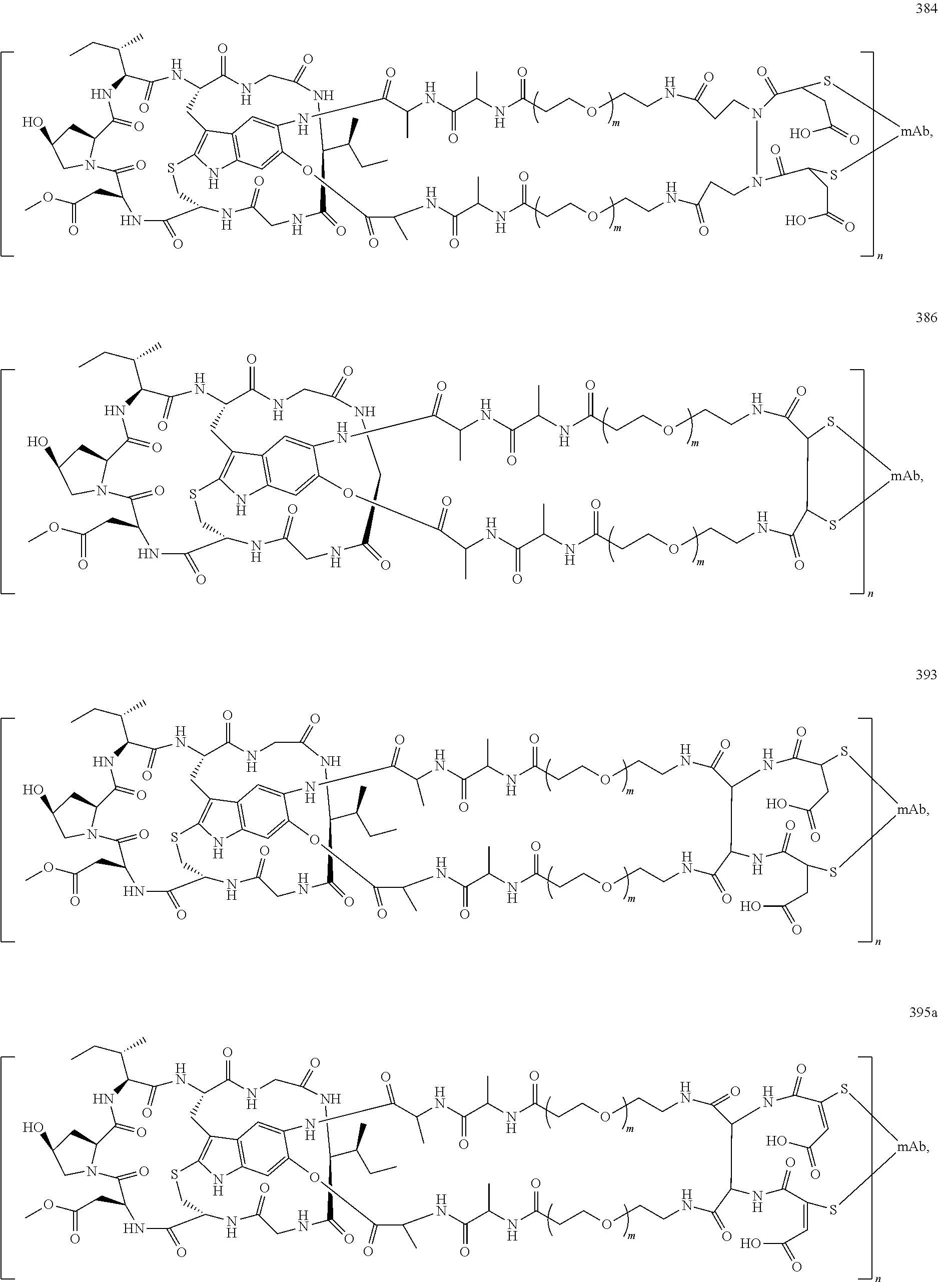 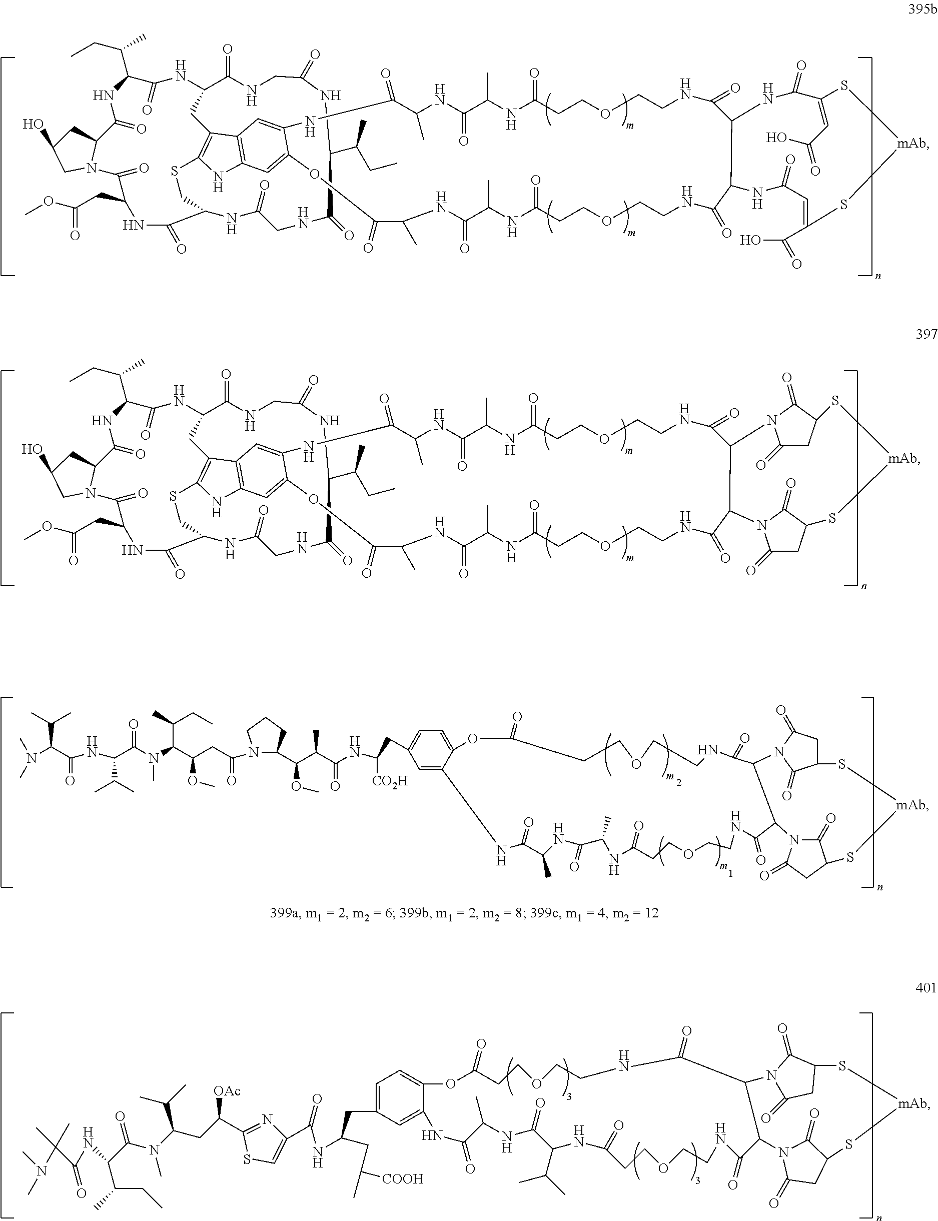 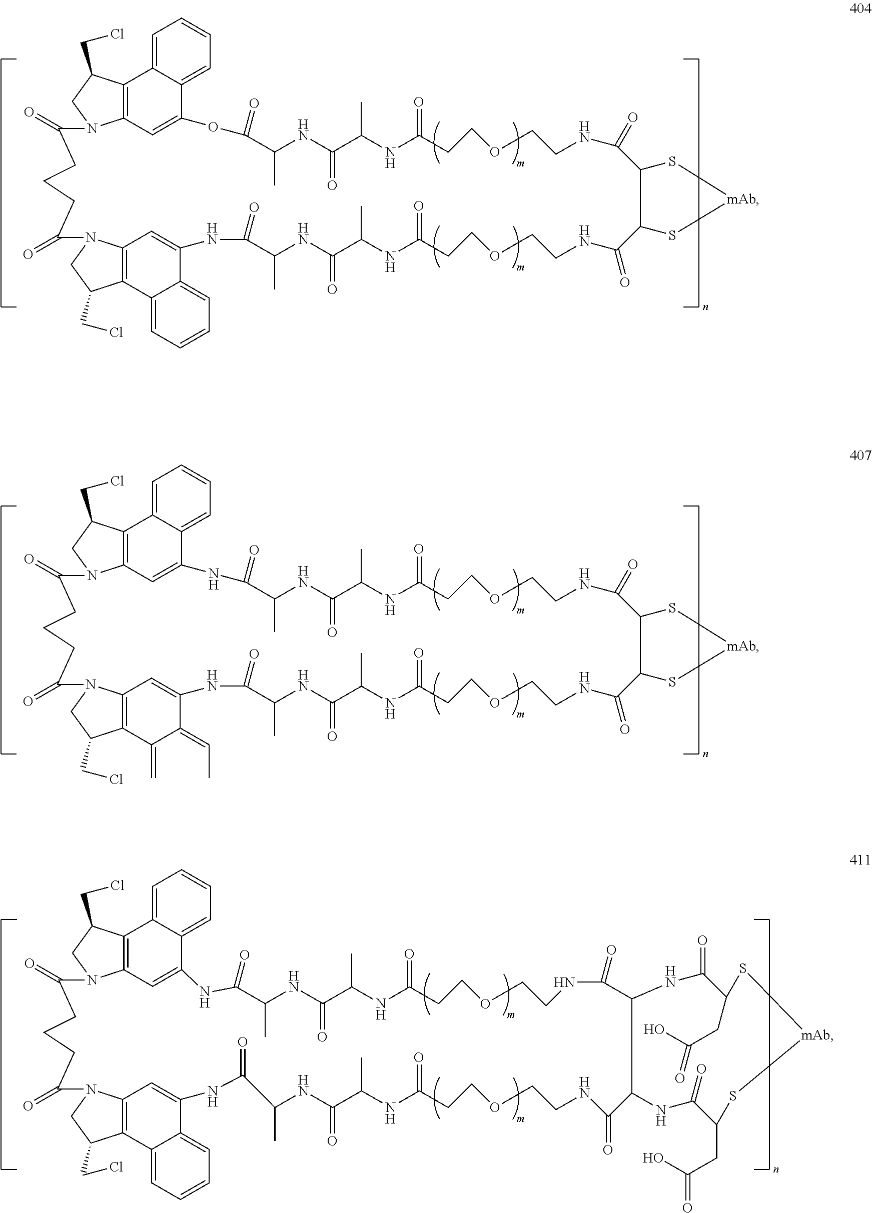 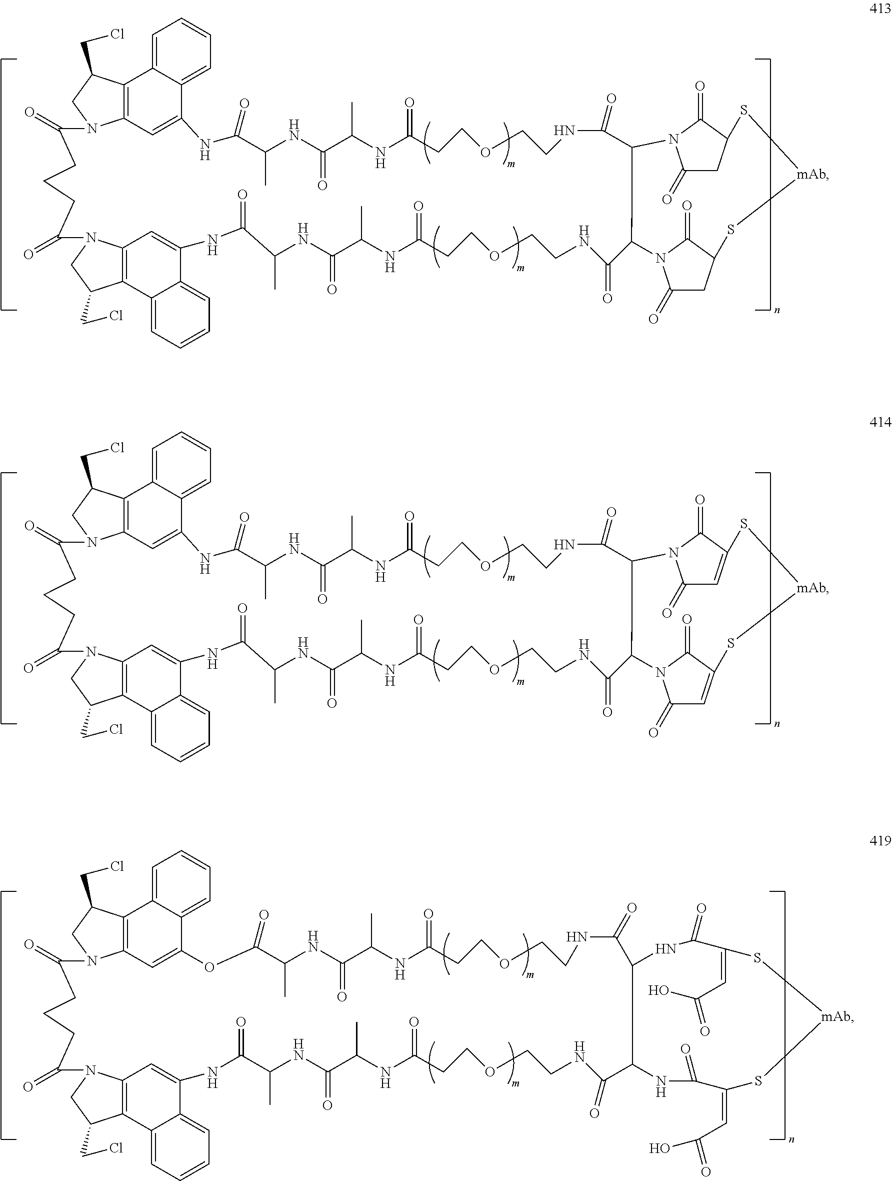 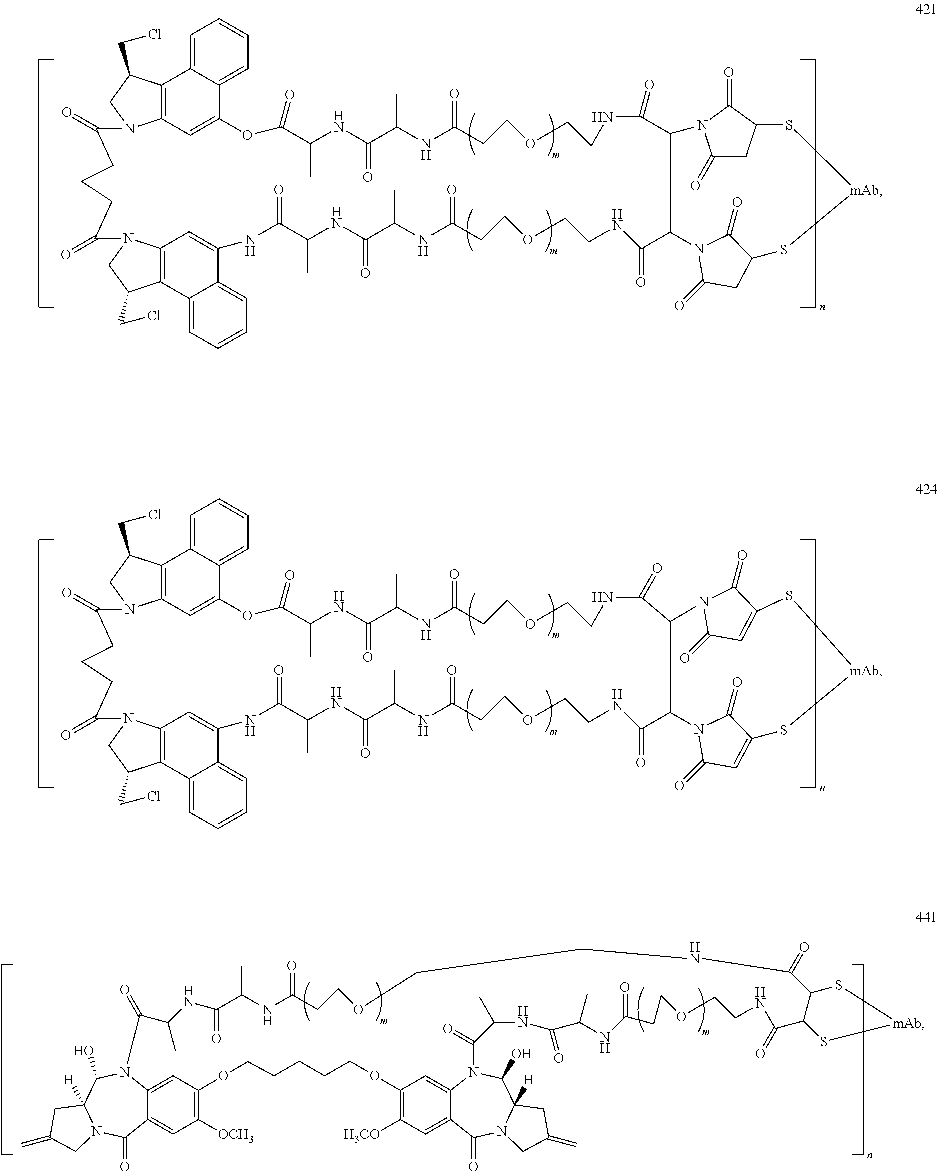 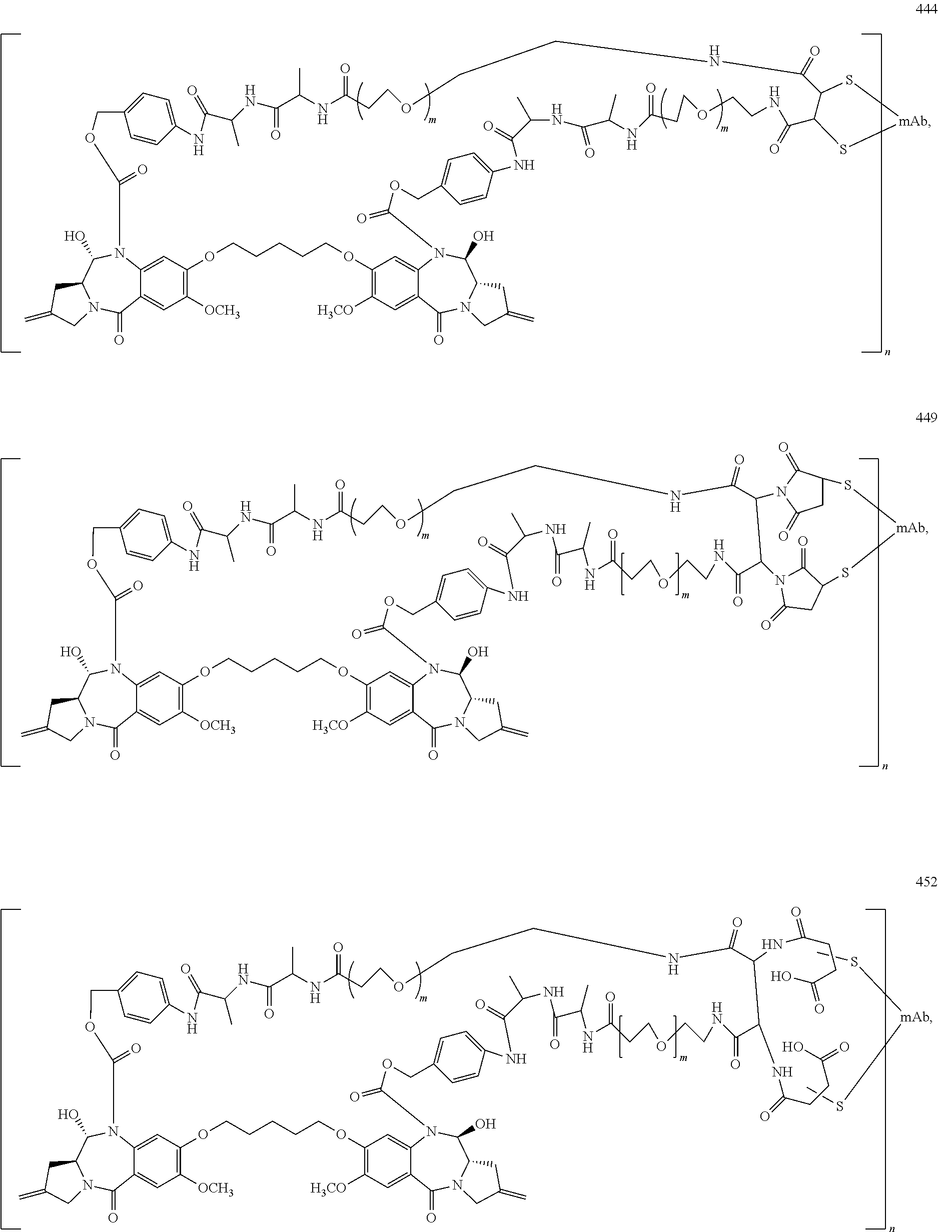 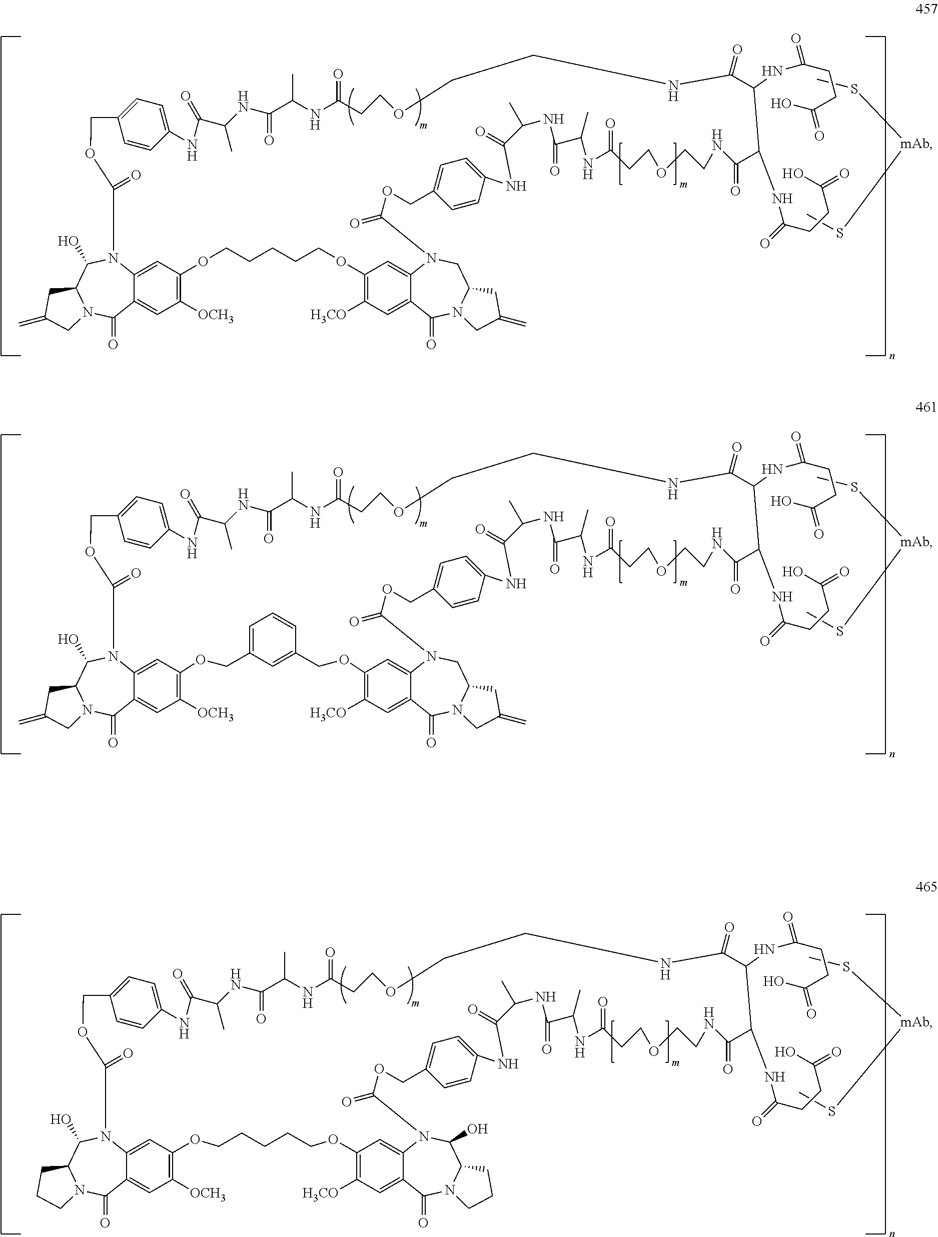 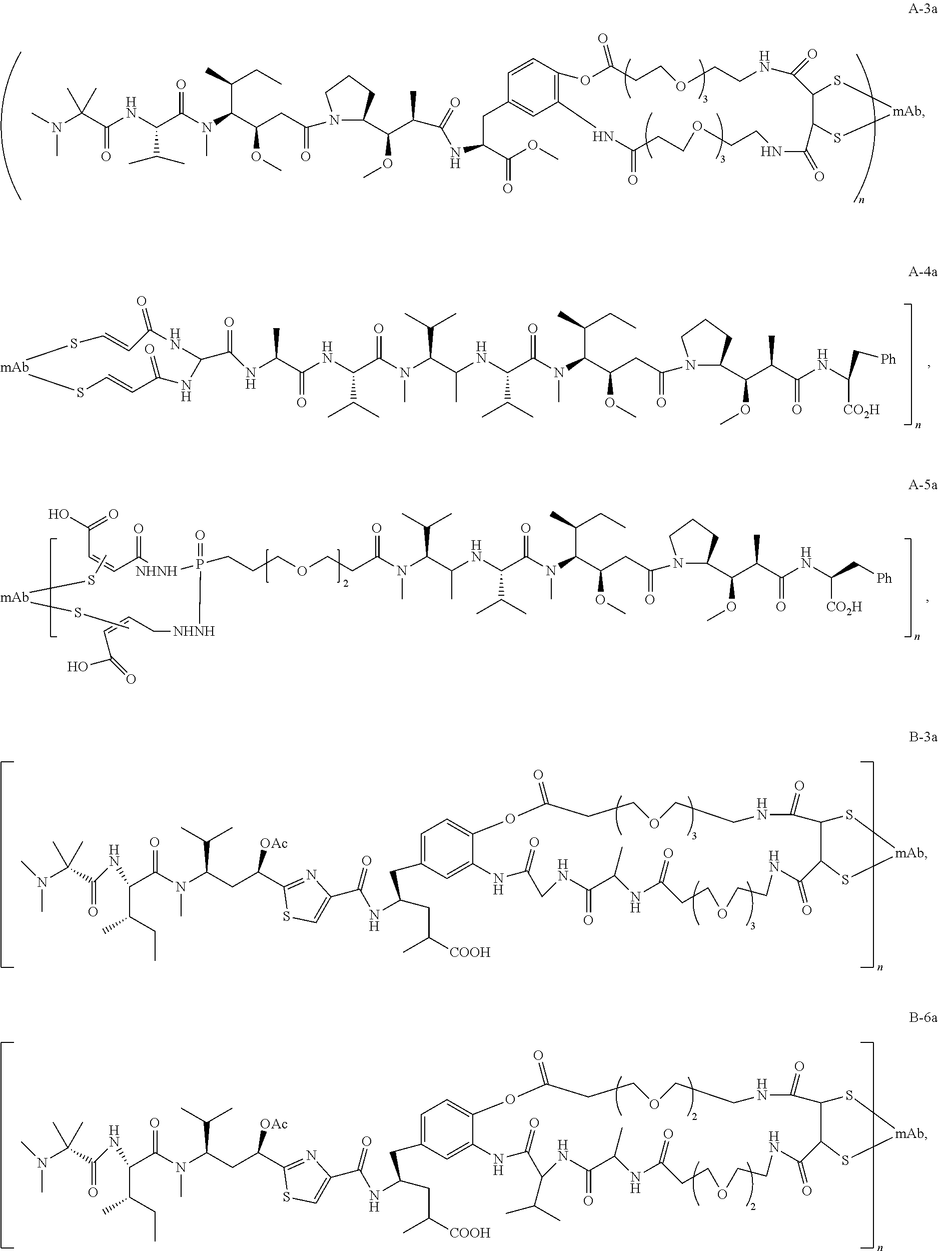 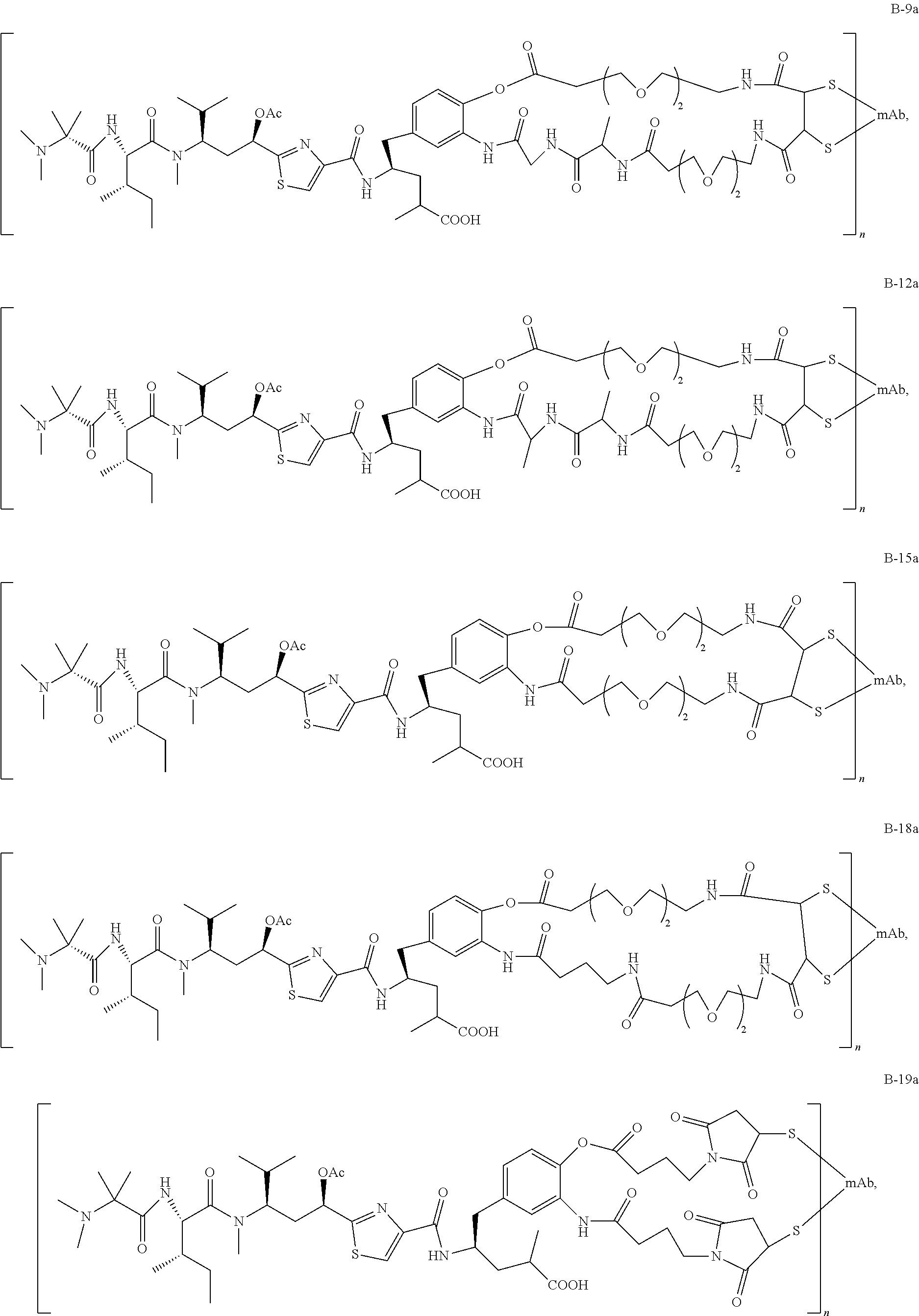 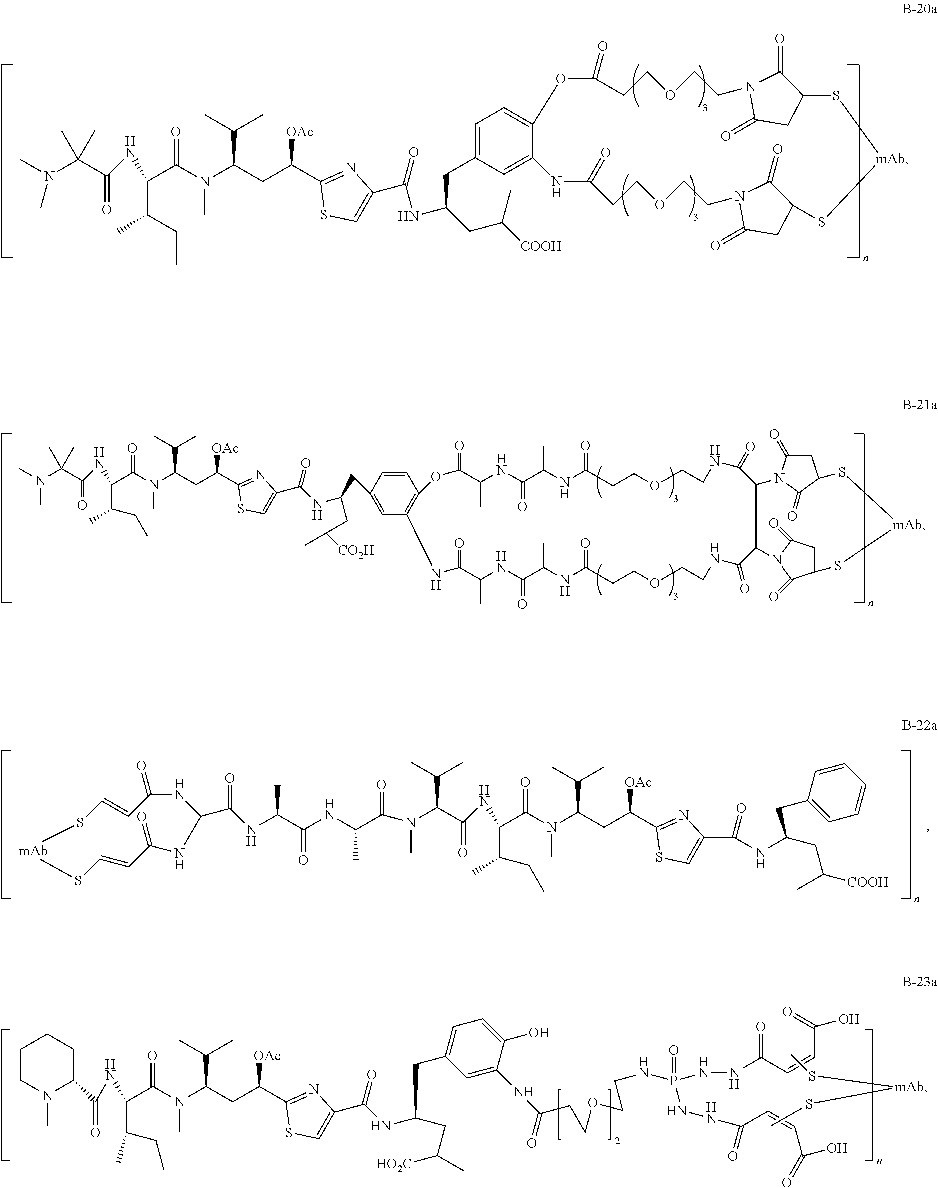 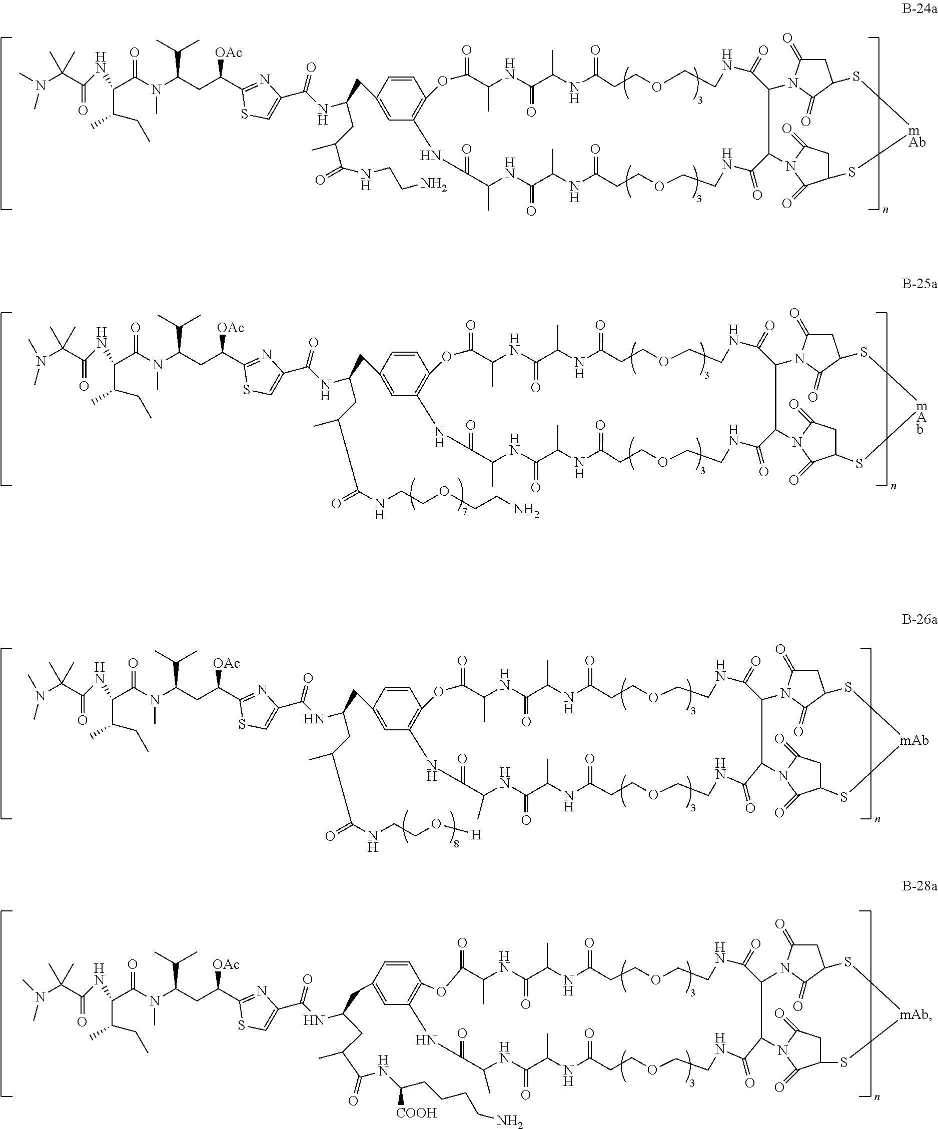 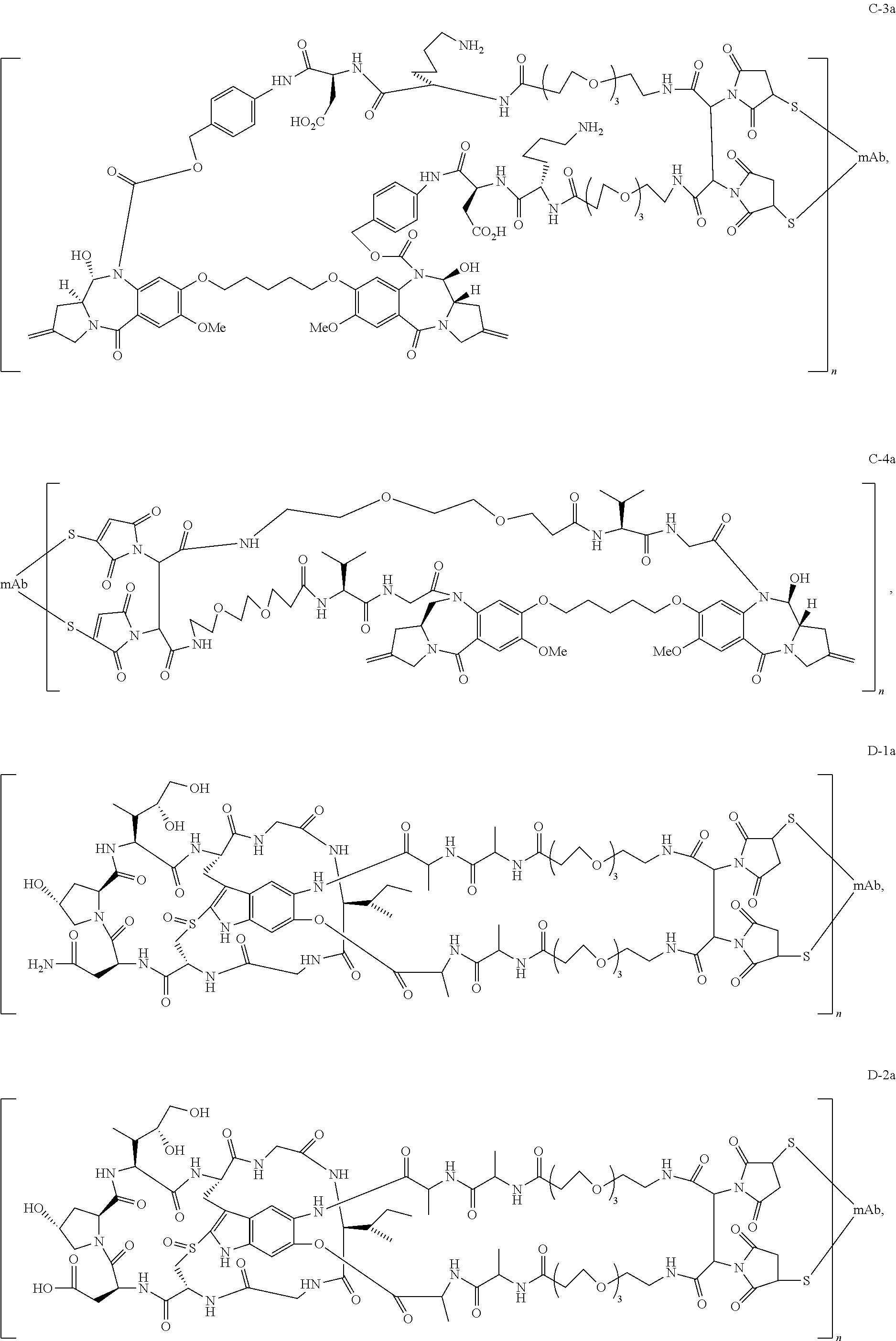
wherein m is 0-20 if not indicated in the formula; mAb, m₁, and n are defined the same as in claim 1.

37. A pharmaceutical composition comprising a therapeutically effective amount of one or more of the conjugate compound of claim 1, and a pharmaceutically acceptable salt, carrier, diluent, or excipient therefor; for treatment or prevention of a cancer, or an autoimmune disease, or an infectious disease.

38. The pharmaceutical composition according to claim 37, comprising 0.1 g/L˜300 g/L of the one or more of the conjugate compound; a buffering agent with pH 4.5 to 7.5 at a concentration of 10 mM-500 nM; 0%-15% of one or more polyols (comprising fructose, mannose, maltose, lactose, arabinose, xylose, ribose, rhamnose, galactose, glucose, sucrose, trehalose, sorbose, melezitose, raffinose, mannitol, xylitol, erythritol, maltitol, lactitol, erythritol, threitol, sorbitol, glycerol, or L-gluconate and its metallic salts); 0-1.0% of a surfactant [selected from polysorbate (comprising polysorbate 20, polysorbate 40, polysorbate 65, polysorbate 80, polysorbate 81, or polysorbate 85), poloxamer (comprising poloxamer 188, poly(ethylene oxide)-poly(propylene oxide), or poloxamer 407 or polyethylene-polypropylene glycol); Triton; sodium dodecyl sulfate; sodium laurel sulfate; sodium octyl glycoside; lauryl-, myristyl-, linoleyl-, or stearyl-sulfobetaine; lauryl-, myristyl-, linoleyl- or stearyl-sarcosine; linoleyl-, myristyl-, or cetyl-betaine; lauroamidopropyl-, cocamidopropyl-, linoleamidopropyl-, myristamidopropyl-, palmidopropyl-, or isostearamidopropyl-betaine (lauroamidopropyl); myristamidopropyl-, palmidopropyl-, or isostearamidopropyl-dimethylamine; sodium methyl cocoyl-, or disodium methyl oleyl-taurate; dodecyl betaine, dodecyl dimethylamine oxide, cocamidopropyl betaine and coco ampho glycinate; MONAQUAT™ series (isostearyl ethylimidonium ethosulfate); polyethyl glycol, polypropyl glycol, and copolymers of ethylene and propylene glycol (Pluronics, PF68)]; 0-5 mg/ml of an antioxidant (selected from ascorbic acid and/or methionine); 0-2 mM of a chelating agent (selected from EDTA or EGTA); 0-5% of a preservative (selected from benzyl alcohol, octadecyldimethylbenzyl ammonium chloride, hexamethonium chloride, benzalkonium chloride, benzethonium chloride, phenol, butyl and benzyl alcohol, alkyl parabens such as methyl or propyl paraben, catechol, resorcinol, cyclohexanol, 3-pentanol, or m-cresol); 0-15% of a free amino acid; and/or a tonicity agent (selected from mannitol, sorbitol, sodium acetate, potassium chloride, sodium phosphate, potassium phosphate, trisodium citrate, or NaCl), wherein the pharmaceutical composition has an osmotic pressure from about 250 to 350 mOsm.

39. The pharmaceutical composition according to claim 37, which is held in a vial, bottle, pre-filled syringe, or pre-filled auto-injector syringe, in a form of a liquid or lyophilized solid.

40. The conjugate compound of claim 1, having in vitro, in vivo or ex vivo cell killing activity.

41. The pharmaceutical composition according to claim 37, further comprising a synergistic agent of a chemotherapeutic agent, a radiation therapy agent, an immunotherapy agent, an autoimmune disorder agent, an anti-infectious agents or another conjugate for synergistically treatment or prevention of a cancer, or an autoimmune disease, or an infectious disease.

42. The pharmaceutical composition according to claim 41, wherein the synergistic agent is one or more of following drugs: Abatacept, Abiraterone acetate, Acetaminophen/hydrocodone, aducanumab, Adalimumab, ADXS31-142, ADXS-HER2, afatinib dimaleate, alemtuzumab, Ali-tretinoin, ado-trastuzumab emtansine, Amphetamine mixed salts (Amphetamine/dextroamphetamine, or Adderall XR), anastrozole, Aripiprazole, Atazanavir, Atezolizumab, Atorvastatin, axitinib, Avelumab, belinostat, Bevacizumab, Cabazitaxel, Cabozantinib, bexarotene, blinatumomab, Bortezomib, bosutinib, brentuximab vedotin, Budesonide, Budesonide/formoterol, Buprenorphine, Capecitabine, carfilzomib, Celecoxib, ceritinib, Cetuximab, Ciclosporin, Cinacalcet, crizotinib, Cosentyx, CTL019, Dabigatran, dabrafenib, Daratumumab, Darbepoetin alfa, Darunavir, imatinib mesylate, dasatinib, denileukin diftitox, Denosumab, Depakote, Dexlansoprazole, Dexmethylphenidate, Dexamethasone, Dignitana DigniCap Cooling System, Dinutuximab, Doxycycline, Duloxetine, Duvelisib, elotuzumab, Emtricitabine/Rilpivirine/Tenofovir disoproxil fumarate, Emtricitbine/tenofovir/efavirenz, Enoxaparin, Enzalutamide, Epoetin alfa, erlotinib, Esomeprazole, Eszopiclone, Etanercept, Everolimus, exemestane, everolimus, Ezetimibe, Ezetimibe/simvastatin, Fenofibrate, Filgrastim, fingolimod, Fluticasone propionate, Fluticasone/salmeterol, fulvestrant, gazyva, gefitinib, Glatiramer, Goserelin acetate, Icotinib, Imatinib, Ibritumomab tiuxetan, ibrutinib, idelalisib, Infliximab, iniparib, Insulin aspart, Insulin detemir, Insulin glargine, Insulin lispro, Interferon beta 1a, Interferon beta 1b, lapatinib, Ipilimumab, Ipratropium bromide/salbutamol, Ixazomi, Kanuma, Lanreotide acetate, lenalidomide, lenaliomide, lenvatinib mesylate, letrozole, Levothyroxine, Levothyroxine, Lidocaine, Linezolid, Liraglutide, Lisdexamfetamine, LN-144, MEDI4736, Memantine, Methylphenidate, Metoprolol, Mekinist, Modafinil, Mometasone, Nilotinib, niraparib, Nivolumab, ofatumumab, obinutuzumab, olaparib, Olmesartan, Olmesartan/hydrochlorothiazide, Omalizumab, Omega-3 fatty acid ethyl esters, Oseltamivir, Oxycodone, palbociclib, Palivizumab, panitumumab, panobinostat, pazopanib, pembrolizumab, Pemetrexed, pertuzumab, Pneumococcal conjugate vaccine, pomalidomide, Pregabalin, ProscaVax, Propranolol, Quetiapine, Rabeprazole, radium 223 chloride, Raloxifene, Raltegravir, ramucirumab, Ranibizumab, regorafenib, Rituximab, Rivaroxaban, romidepsin, Rosuvastatin, ruxolitinib phosphate, Salbutamol, Sevelamer, Sildenafil, siltuximab, Sitagliptin, Sitagliptin/metformin, Solifenacin, solanezumab, Sorafenib, Sunitinib, Tadalafil, tamoxifen, Tafinlar, talazoparib, Telaprevir, temsirolimus, Tenofovir/emtricitabine, Testosterone gel, Thalidomide, Tiotropium bromide, toremifene, trametinib, Trastuzumab, Tretinoin, Ustekinumab, Valsartan, veliparib, vandetanib, vemurafenib, venetoclax, vorinostat, ziv-aflibercept, Zostavax, and analogs, derivatives, pharmaceutically acceptable salts, carriers, diluents, or excipients thereof, or a combination thereof.
